SEQUENCE LISTING (143 sequences)

SEQ ID NO: 1 - DNA, 297 bp, Human papillomavirus
  CDS (1)..(297)
atgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcaggataagctt

SEQ ID NO: 2 - protein, 99 aa, Human papillomavirus
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQDKL

SEQ ID NO: 3 - protein, 98 aa, Human papillomavirus
MHGDTPTLHEYMLDLQPETTDLYGYEGLNDSSEEEDEIDGPAGQAEPDRAHYNIVTFCCKCDSTLRLCVQSTHVDIRTLEDLLMGTLGIVCPICSQKP

SEQ ID NO: 4 - DNA, 477 bp, Human papillomavirus
  CDS (1)..(474)
atgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 5 - protein, 158 aa, Human papillomavirus
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 6 - protein, 151 aa, Human papillomavirus
MFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 7 - DNA, 1698 bp, Influenza A virus
atgaaggcaaacctactggtcctgttaagtgcacttgcagctgcagatgcagacacaatatgtataggctaccatgcgaacaattcaaccgacactgttgacacagtactcgagaagaatgtgacagtgacacactctgttaacctgctcgaagacagccacaacggaaaactatgtagattaaaaggaatagccccactacaattggggaaatgtaacatcgccggatggctcttgggaaacccagaatgcgacccactgcttccagtgagatcatggtcctacattgtagaaacaccaaactctgagaatggaatatgttatccaggagatttcatcgactatgaggagctgagggagcaattgagctcagtgtcatcattcgaaagattcgaaatatttcccaaagaaagctcatggcccaaccacaacacaaacggagtaacggcagcatgctcccatgaggggaaaagcagtttttacagaaatttgctatggctgacggagaaggagggctcatacccaaagctgaaaaattcttatgtgaacaaaaaagggaaagaagtccttgtactgtggggtattcatcacccgcctaacagtaaggaacaacagaatatctatcagaatgaaaatgcttatgtctctgtagtgacttcaaattataacaggagatttaccccggaaatagcagaaagacccaaagtaagagatcaagctgggaggatgaactattactggaccttgctaaaacccggagacacaataatatttgaggcaaatggaaatctaatagcaccaatgtatgctttcgcactgagtagaggctttgggtccggcatcatcacctcaaacgcatcaatgcatgagtgtaacacgaagtgtcaaacacccctgggagctataaacagcagtctcccttaccagaatatacacccagtcacaataggagagtgcccaaaatacgtcaggagtgccaaattgaggatggttacaggactaaggaacactccgtccattcaatccagaggtctatttggagccattgccggttttattgaagggggatggactggaatgatagatggatggtatggttatcatcatcagaatgaacagggatcaggctatgcagcggatcaaaaaagcacacaaaatgccattaacgggattacaaacaaggtgaacactgttatcgagaaaatgaacattcaattcacagctgtgggtaaagaattcaacaaattagaaaaaaggatggaaaatttaaataaaaaagttgatgatggatttctggacatttggacatataatgcagaattgttagttctactggaaaatgaaaggactctggatttccatgactcaaatgtgaagaatctgtatgagaaagtaaaaagccaattaaagaataatgccaaagaaatcggaaatggatgttttgagttctaccacaagtgtgacaatgaatgcatggaaagtgtaagaaatgggacttatgattatcccaaatattcagaagagtcaaagttgaacagggaaaaggtagatggagtgaaattggaatcaatggggatctatcagattctggcgatctactcaactgtcgccagttcactggtgcttttggtctccctgggggcaatcagtttctggatgtgttctaatggatctttgcagtgcagaatatgcatctga

SEQ ID NO: 8 - protein, 565 aa, Influenza A virus
MKANLLVLLSALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNIYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNTPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 9 - protein, 386 aa, Unknown
  Description of Unknown Representative ovalbumin polypeptide
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKTFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 10 - DNA, 501 bp, Artificial Sequence
  Description of Artificial Sequence Synthetic polynucleotide
atggcggcccccggcgcccggcggccgctgctcctgctgctgctggcaggccttgcacatggcgcctcagcactctttgaggatctaatcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgttaccttttgttgcaagtgtgactctacgcttcggttgtgcgtacaaagcacacacgtagacattcgtactttggaagacctgttaatgggcacactaggaattgtgtgccccatctgttctcaggatcttaacaacatgttgatccccattgctgtgggcggtgccctggcagggctggtcctcatcgtcctcattgcctacctcattggcaggaagaggagtcacgccggctatcagaccatctag

SEQ ID NO: 11 - protein, 166 aa, Artificial Sequence
  Description of Artificial Sequence Synthetic polypeptide
MAAPGARRPLLLLLLAGLAHGASALFEDLIMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQDLNNMLIPIAVGGALAGLVLIVLIAYLIGRKRSHAGYQTI

SEQ ID NO: 12 - DNA, 5915 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattcatggcggcccccggcgcccggcggccgctgctcctgctgctgctggcaggccttgcacatggcgcctcagcactctttgaggatctaatcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgttaccttttgttgcaagtgtgactctacgcttcggttgtgcgtacaaagcacacacgtagacattcgtactttggaagacctgttaatgggcacactaggaattgtgtgccccatctgttctcaggatcttaacaacatgttgatccccattgctgtgggcggtgccctggcagggctggtcctcatcgtcctcattgcctacctcattggcaggaagaggagtcacgccggctatcagaccatctagggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 13 - DNA, 1878 bp, Mycobacterium tuberculosis
atggctcgtgcggtcgggatcgacctcgggaccaccaactccgtcgtctcggttctggaaggtggcgacccggtcgtcgtcgccaactccgagggctccaggaccaccccgtcaattgtcgcgttcgcccgcaacggtgaggtgctggtcggccagcccgccaagaaccaggcagtgaccaacgtcgatcgcaccgtgcgctcggtcaagcgacacatgggcagcgactggtccatagagattgacggcaagaaatacaccgcgccggagatcagcgcccgcattctgatgaagctgaagcgcgacgccgaggcctacctcggtgaggacattaccgacgcggttatcacgacgcccgcctacttcaatgacgcccagcgtcaggccaccaaggacgccggccagatcgccggcctcaacgtgctgcggatcgtcaacgagccgaccgcggccgcgctggcctacggcctcgacaagggcgagaaggagcagcgaatcctggtcttcgacttgggtggtggcactttcgacgtttccctgctggagatcggcgagggtgtggttgaggtccgtgccacttcgggtgacaaccacctcggcggcgacgactgggaccagcgggtcgtcgattggctggtggacaagttcaagggcaccagcggcatcgatctgaccaaggacaagatggcgatgcagcggctgcgggaagccgccgagaaggcaaagatcgagctgagttcgagtcagtccacctcgatcaacctgccctacatcaccgtcgacgccgacaagaacccgttgttcttagacgagcagctgacccgcgcggagttccaacggatcactcaggacctgctggaccgcactcgcaagccgttccagtcggtgatcgctgacaccggcatttcggtgtcggagatcgatcacgttgtgctcgtgggtggttcgacccggatgcccgcggtgaccgatctggtcaaggaactcaccggcggcaaggaacccaacaagggcgtcaaccccgatgaggttgtcgcggtgggagccgctctgcaggccggcgtcctcaagggcgaggtgaaagacgttctgctgcttgatgttaccccgctgagcctgggtatcgagaccaagggcggggtgatgaccaggctcatcgagcgcaacaccacgatccccaccaagcggtcggagactttcaccaccgccgacgacaaccaaccgtcggtgcagatccaggtctatcagggggagcgtgagatcgccgcgcacaacaagttgctcgggtccttcgagctgaccggcatcccgccggcgccgcgggggattccgcagatcgaggtcactttcgacatcgacgccaacggcattgtgcacgtcaccgccaaggacaagggcaccggcaaggagaacacgatccgaatccaggaaggctcgggcctgtccaaggaagacattgaccgcatgatcaaggacgccgaagcgcacgccgaggaggatcgcaagcgtcgcgaggaggccgatgttcgtaatcaagccgagacattggtctaccagacggagaagttcgtcaaagaacagcgtgaggccgagggtggttcgaaggtacctgaagacacgctgaacaaggttgatgccgcggtggcggaagcgaaggcggcacttggcggatcggatatttcggccatcaagtcggcgatggagaagctgggccaggagtcgcaggctctggggcaagcgatctacgaagcagctcaggctgcgtcacaggccactggcgctgcccaccccggcggcgagccgggcggtgcccaccccggctcggctgatgacgttgtggacgcggaggtggtcgacgacggccgggaggccaagtga

SEQ ID NO: 14 - protein, 625 aa, Mycobacterium tuberculosis
MARAVGIDLGTTNSVVSVLEGGDPVVVANSEGSRTTPSIVAFARNGEVLVGQPAKNQAVTNVDRTVRSVKRHMGSDWSIEIDGKKYTAPEISARILMKLKRDAEAYLGEDITDAVITTPAYFNDAQRQATKDAGQIAGLNVLRIVNEPTAAALAYGLDKGEKEQRILVFDLGGGTFDVSLLEIGEGVVEVRATSGDNHLGGDDWDQRVVDWLVDKFKGTSGIDLTKDKMAMQRLREAAEKAKIELSSSQSTSINLPYITVDADKNPLFLDEQLTRAEFQRITQDLLDRTRKPFQSVIADTGISVSEIDHVVLVGGSTRMPAVTDLVKELTGGKEPNKGVNPDEVVAVGAALQAGVLKGEVKDVLLLDVTPLSLGIETKGGVMTRLIERNTTIPTKRSETFTTADDNQPSVQIQVYQGEREIAAHNKLLGSFELTGIPPAPRGIPQIEVTFDIDANGIVHVTAKDKGTGKENTIRIQEGSGLSKEDIDRMIKDAEAHAEEDRKRREEADVRNQAETLVYQTEKFVKEQREAEGGSKVPEDTLNKVDAAVAEAKAALGGSDISAIKSAMEKLGQESQALGQAIYEAAQAASQATGAAHPGGEPGGAHPGSADDVVDAEVVDDGREAK

SEQ ID NO: 15 - DNA, 2104 bp, Artificial Sequence
  Description of Artificial Sequence Synthetic polynucleotide
atgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcaaggatccatggctcgtgcggtcgggatcgacctcgggaccaccaactccgtcgtctcggttctggaaggtggcgacccggtcgtcgtcgccaactccgagggctccaggaccaccccgtcaattgtcgcgttcgcccgcaacggtgaggtgctggtcggccagcccgccaagaaccaggcagtgaccaacgtcgatcgcaccgtgcgctcggtcaagcgacacatgggcagcgactggtccatagagattgacggcaagaaatacaccgcgccggagatcagcgcccgcattctgatgaagctgaagcgcgacgccgaggcctacctcggtgaggacattaccgacgcggttatcacgacgcccgcctacttcaatgacgcccagcgtcaggccaccaaggacgccggccagatcgccggcctcaacgtgctgcggatcgtcaacgagccgaccgcggccgcgctggcctacggcctcgacaagggcgagaaggagcagcgaatcctggtcttcgacttgggtggtggcactttcgacgtttccctgctggagatcggcgagggtgtggttgaggtccgtgccacttcgggtgacaaccacctcggcggcgacgactgggaccagcgggtcgtcgattggctggtggacaagttcaagggcaccagcggcatcgatctgaccaaggacaagatggcgatgcagcggctgcgggaagccgccgagaaggcaaagatcgagctgagttcgagtcagtccacctcgatcaacctgccctacatcaccgtcgacgccgacaagaacccgttgttcttagacgagcagctgacccgcgcggagttccaacggatcactcaggacctgctggaccgcactcgcaagccgttccagtcggtgatcgctgacaccggcatttcggtgtcggagatcgatcacgttgtgctcgtgggtggttcgacccggatgcccgcggtgaccgatctggtcaaggaactcaccggcggcaaggaacccaacaagggcgtcaaccccgatgaggttgtcgcggtgggagccgctctgcaggccggcgtcctcaagggcgaggtgaaagacgttctgctgcttgatgttaccccgctgagcctgggtatcgagaccaagggcggggtgatgaccaggctcatcgagcgcaacaccacgatccccaccaagcggtcggagactttcaccaccgccgacgacaaccaaccgtcggtgcagatccaggtctatcagggggagcgtgagatcgccgcgcacaacaagttgctcgggtccttcgagctgaccggcatcccgccggcgccgcgggggattccgcagatcgaggtcactttcgacatcgacgccaacggcattgtgcacgtcaccgccaaggacaagggcaccggcaaggagaacacgatccgaatccaggaaggctcgggcctgtccaaggaagacattgaccgcatgatcaaggacgccgaagcgcacgccgaggaggatcgcaagcgtcgcgaggaggccgatgttcgtaatcaagccgagacattggtctaccagacggagaagttcgtcaaagaacagcgtgaggccgagggtggttcgaaggtacctgaagacacgctgaacaaggttgatgccgcggtggcggaagcgaaggcggcacttggcggatcggatatttcggccatcaagtcggcgatggagaagctgggccaggagtcgcaggctctggggcaagcgatctacgaagcagctcaggctgcgtcacaggccactggcgctgcccaccccggctcggctgatgaaagca

SEQ ID NO: 16 - protein, 701 aa, Artificial Sequence
  Description of Artificial Sequence Synthetic polypeptide
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQGSMARAVGIDLGTTNSVVSVLEGGDPVVVANSEGSRTTPSIVAFARNGEVLVGQPAKNQAVTNVDRTVRSVKRHMGSDWSIEIDGKKYTAPEISARILMKLKRDAEAYLGEDITDAVITTPAYFNDAQRQATKDAGQIAGLNVLRIVNEPTAAALAYGLDKGEKEQRILVFDLGGGTFDVSLLEIGEGVVEVRATSGDNHLGGDDWDQRVVDWLVDKFKGTSGIDLTKDKMAMQRLREAAEKAKIELSSSQSTSINLPYITVDADKNPLFLDEQLTRAEFQRITQDLLDRTRKPFQSVIADTGISVSEIDHVVLVGGSTRMPAVTDLVKELTGGKEPNKGVNPDEVVAVGAALQAGVLKGEVKDVLLLDVTPLSLGIETKGGVMTRLIERNTTIPTKRSETFTTADDNQPSVQIQVYQGEREIAAHNKLLGSFELTGIPPAPRGIPQIEVTFDIDANGIVHVTAKDKGTGKENTIRIQEGSGLSKEDIDRMIKDAEAHAEEDRKRREEADVRNQAETLVYQTEKFVKEQREAEGGSKVPEDTLNKVDAAVAEAKAALGGSDISAIKSAMEKLGQESQALGQAIYEAAQAASQATGAAHPGSADES

SEQ ID NO: 17 - DNA, 2760 bp, Pseudomonas aeruginosa
ctgcagctggtcaggccgtttccgcaacgcttgaagtcctggccgatataccggcagggccagccatcgttcgacgaataaagccacctcagccatgatgccctttccatccccagcggaaccccgacatggacgccaaagccctgctcctcggcagcctctgcctggccgccccattcgccgacgcggcgacgctcgacaatgctctctccgcctgcctcgccgcccggctcggtgcaccgcacacggcggagggccagttgcacctgccactcacccttgaggcccggcgctccaccggcgaatgcggctgtacctcggcgctggtgcgatatcggctgctggccaggggcgccagcgccgacagcctcgtgcttcaagagggctgctcgatagtcgccaggacacgccgcgcacgctgaccctggcggcggacgccggcttggcgagcggccgcgaactggtcgtcaccctgggttgtcaggcgcctgactgacaggccgggctgccaccaccaggccgagatggacgccctgcatgtatcctccgatcggcaagcctcccgttcgcacattcaccactctgcaatccagttcataaatcccataaaagccctcttccgctccccgccagcctccccgcatcccgcaccctagacgccccgccgctctccgccggctcgcccgacaagaaaaaccaaccgctcgatcagcctcatccttcacccatcacaggagccatcgcgatgcacctgataccccattggatccccctggtcgccagcctcggcctgctcgccggcggctcgtccgcgtccgccgccgaggaagccttcgacctctggaacgaatgcgccaaagcctgcgtgctcgacctcaaggacggcgtgcgttccagccgcatgagcgtcgacccggccatcgccgacaccaacggccagggcgtgctgcactactccatggtcctggagggcggcaacgacgcgctcaagctggccatcgacaacgccctcagcatcaccagcgacggcctgaccatccgcctcgaaggcggcgtcgagccgaacaagccggtgcgctacagctacacgcgccaggcgcgcggcagttggtcgctgaactggctggtaccgatcggccacgagaagccctcgaacatcaaggtgttcatccacgaactgaacgccggcaaccagctcagccacatgtcgccgatctacaccatcgagatgggcgacgagttgctggcgaagctggcgcgcgatgccaccttcttcgtcagggcgcacgagagcaacgagatgcagccgacgctcgccatcagccatgccggggtcagcgtggtcatggcccagacccagccgcgccgggaaaagcgctggagcgaatgggccagcggcaaggtgttgtgcctgctcgacccgctggacggggtctacaactacctcgcccagcaacgctgcaacctcgacgatacctgggaaggcaagatctaccgggtgctcgccggcaacccggcgaagcatgacctggacatcaaacccacggtcatcagtcatcgcctgcactttcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacgccgacgtggtgagcctgacctgcccggtcgccgccggtgaatgcgcgggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgcgcgaggacctgaagtaactgccgcgaccggccggctcccttcgcaggagccggccttctcggggcctggccatacatcaggttttcctgatgccagcccaatcgaatatgaattc

SEQ ID NO: 18 - protein, 638 aa, Pseudomonas aeruginosa
MHLIPHWIPLVASLGLLAGGSSASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQTQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 19 - protein, 171 aa, Pseudomonas aeruginosa
RLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNW

SEQ ID NO: 20 - DNA, 870 bp, Artificial Sequence
  Description of Artificial Sequence Synthetic polynucleotide
atgcgcctgcactttcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacgccgacgtggtgagcctgacctgcccggtcgccgccggtgaatgcgcgggcccggcggacagcggcgacgccctgctggagcgcaactatcccactggcgcggagttcctcggcgacggcggcgacgtcagcttcagcacccgcggcacgcagaacgaattcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcaaggatccgagctcggtaccaagcttaagtttaaaccgctgatcagcctcgactgtgccttctag

SEQ ID NO: 21 - protein, 280 aa, Artificial Sequence
  Description of Artificial Sequence Synthetic polypeptide
MRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNEFMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQGSELGTKLKFK

SEQ ID NO: 22 - DNA, 1257 bp, Artificial Sequence
  Description of Artificial Sequence Synthetic polynucleotide
atgacctctcgccgctccgtgaagtcgggtccgcgggaggttccgcgcgatgagtacgaggatctgtactacaccccgtcttcaggtatggcgagtcccgatagtccgcctgacacctcccgccgtggcgccctacagacacgctcgcgccagaggggcgaggtccgtttcgtccagtacgacgagtcggattatgccctctacgggggctcgtcttccgaagacgacgaacacccggaggtcccccggacgcggcgtcccgtttccggggcggttttgtccggcccggggcctgcgcgggcgcctccgccacccgctgggtccggaggggccggacgcacacccaccaccgccccccgggccccccgaacccagcgggtggcgtctaaggcccccgcggccccggcggcggagaccacccgcggcaggaaatcggcccagccagaatccgccgcactcccagacgcccccgcgtcgacggcgccaacccgatccaagacacccgcgcaggggctggccagaaagctgcactttagcaccgcccccccaaaccccgacgcgccatggaccccccgggtggccggctttaacaagcgcgtcttctgcgccgcggtcgggcgcctggcggccatgcatgcccggatggcggctgtccagctctgggacatgtcgcgtccgcgcacagacgaagacctcaacgaactccttggcatcaccaccatccgcgtgacggtctgcgagggcaaaaacctgcttcagcgcgccaacgagttggtgaatccagacgtggtgcaggacgtcgacgcggccacggcgactcgagggcgttctgcggcgtcgcgccccaccgagcgacctcgagccccagcccgctccgcttctcgccccagacggcccgtcgagggtaccgagctcggatccatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcaggataagcttaagtttaaaccgctgatcagcctcgactgtgccttctag

SEQ ID NO: 23 - DNA, 1254 bp, Homo sapiens
atgctgctatccgtgccgctgctgctcggcctcctcggcctggccgtcgccgagcccgccgtctacttcaaggagcagtttctggacggagacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctgtag

SEQ ID NO: 24 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 25 - protein, 170 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTH

SEQ ID NO: 26 - protein, 109 aa, Homo sapiens
LYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQ

SEQ ID NO: 27 - protein, 138 aa, Homo sapiens
IDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 28 - DNA, 1254 bp, Homo sapiens; identical to SEQ ID NO: 23

SEQ ID NO: 29 - DNA, 540 bp, Homo sapiens
atgctgctatccgtgccgctgctgctcggcctcctcggcctggccgtcgccgagcccgccgtctacttcaaggagcagtttctggacggagacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaac

SEQ ID NO: 30 - DNA, 267 bp, Homo sapiens
acctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccct

SEQ ID NO: 31 - DNA, 444 bp, Homo sapiens
gagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctg

SEQ ID NO: 32 - DNA, 5970 bp, Artificial Sequence
  Description of Artificial Sequence Synthetic polynucleotide
gctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccagggcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacatgctgctatccgtgccgctgctgctcggcctcctcggcctggccgtcgccgagcctgccgtctacttcaaggagcagtttctggacggggacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctggaattcatgcatggagatacacctacattgcatgaatatatgttagatttgcaaccagagacaactgatctctacggttatgggcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataaggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccatag

SEQ ID NO: 33 - DNA, 903 bp, Herpes simplex virus
  CDS (1)..(903)
atgacctctcgccgctccgtgaagtcgggtccgcgggaggttccgcgcgatgagtacgaggatctgtactacaccccgtcttcaggtatggcgagtcccgatagtccgcctgacacctcccgccgtggcgccctacagacacgctcgcgccagaggggcgaggtccgtttcgtccagtacgacgagtcggattatgccctctacgggggctcgtcttccgaagacgacgaacacccggaggtcccccggacgcggcgtcccgtttccggggcggttttgtccggcccggggcctgcgcgggcgcctccgccacccgctgggtccggaggggccggacgcacacccaccaccgccccccgggccccccgaacccagcgggtggcgtctaaggcccccgcggccccggcggcggagaccacccgcggcaggaaatcggcccagccagaatccgccgcactcccagacgcccccgcgtcgacggcgccaacccgatccaagacacccgcgcaggggctggccagaaagctgcactttagcaccgcccccccaaaccccgacgcgccatggaccccccgggtggccggctttaacaagcgcgtcttctgcgccgcggtcgggcgcctggcggccatgcatgcccggatggcggctgtccagctctgggacatgtcgcgtccgcgcacagacgaagacctcaacgaactccttggcatcaccaccatccgcgtgacggtctgcgagggcaaaaacctgcttcagcgcgccaacgagttggtgaatccagacgtggtgcaggacgtcgacgcggccacggcgactcgagggcgttctgcggcgtcgcgccccaccgagcgacctcgagccccagcccgctccgcttctcgccccagacggcccgtcgag

SEQ ID NO: 34 - DNA, 1257 bp, Artificial Sequence; identical to SEQ ID NO: 22
  Description of Artificial Sequence Synthetic polynucleotide

SEQ ID NO: 35 - DNA, 750 bp, Artificial Sequence
  Description of Artificial Sequence Synthetic polynucleotide
atgggggattctgaaaggcggaaatcggaacggcgtcgttcccttggatatccctctgcatatgatgacgtctcgattcctgctcgcagaccatcaacacgtactcagcgaaatttaaaccaggatgatttgtcaaaacatggaccatttaccgaccatccaacacaaaaacataaatcggcgaaagccgtatcggaagacgtttcgtctaccacccggggtggctttacaaacaaaccccgtaccaagcccggggtcagagctgtacaaagtaataaattcgctttcagtacggctccttcatcagcatctagcacttggagatcaaatacagtggcatttaatcagcgtatgttttgcggagcggttgcaactgtggctcaatatcacgcataccaaggcgcgctcgccctttggcgtcaagatcctccgcgaacaaatgaagaattagatgcatttctttccagagctgtcattaaaattaccattcaagagggtccaaatttgatgggggaagccgaaacctgtgcccgcaaactattggaagagtctggattatcccaggggaacgagaacgtaaagtccaaatctgaacgtacaaccaaatctgaacgtacaagacgcggcggtgaaattgaaatcaaatcgccagatccgggatctcatcgtacacataaccctcgcactcccgcaacttcgcgtcgccatcattcatccgcccgcggatatcgtagcagtgatagcgaataa

SEQ ID NO: 36 - protein, 301 aa, Herpes simplex virus
MTSRRSVKSGPREVPRDEYEDLYYTPSSGMASPDSPPDTSRRGALQTRSRQRGEVRFVQYDESDYALYGGSSSEDDEHPEVPRTRRPVSGAVLSGPGPARAPPPPAGSGGAGRTPTTAPRAPRTQRVASKAPAAPAAETTRGRKSAQPESAALPDAPASTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVE

SEQ ID NO: 37 - protein, 418 aa, Herpes simplex virus
MTSRRSVKSGPREVPRDEYEDLYYTPSSGMASPDSPPDTSRRGALQTRSRQRGEVRFVQYDESDYALYGGSSSEDDEHPEVPRTRRPVSGAVLSGPGPARAPPPPAGSGGAGRTPTTAPRAPRTQRVASKAPAAPAAETTRGRKSAQPESAALPDAPASTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVEGTELGSMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQDKLKFKPLISLDCAF

SEQ ID NO: 38 - protein, 249 aa, Artificial Sequence
  Description of Artificial Sequence Synthetic polypeptide
MGDSERRKSERRRSLGYPSAYDDVSIPARRPSTRTQRNLNQDDLSKHGPFTDHPTQKHKSAKAVSEDVSSTTRGGFTNKPRTKPGVRAVQSNKFAFSTAPSSASSTWRSNTVAFNQRMFCGAVATVAQYHAYQGALALWRQDPPRTNEELDAFLSRAVIKITIQEGPNLMGEAETCARKLLEESGLSQGNENVKSKSERTTKSERTRRGGEIEIKSPDPGSHRTHNPRTPATSRRHHSSARGYRSSDSE

SEQ ID NO: 39 - protein, 96 aa, Herpes simplex virus
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQ

SEQ ID NO: 40 - DNA, 5431 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 41 - DNA, 4479 bp, Artificial Sequence
  Description of Artificial Sequence Synthetic polynucleotide
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattcacgtgggcccggtaccgtatactctagagcggccgcggatccagatctttttccctcgccaaaaattatggggacatcatgaagccccttgagcatctgacttctggctaataaaggaaatttatttcattgcaatagtgtgttggaattttttgtgtctctcactcggaaggacatatgggagggcaaatcatttaaaacatcagaatcagtatttggtttagagtttggcaacatatgccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccagggcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 42 - DNA, 21 bp, Artificial Sequence
  Description of Artificial Sequence Synthetic oligonucleotide
ugccuacgaacucuucacctt

SEQ ID NO: 43 - DNA, 21 bp, Artificial Sequence
  Description of Artificial Sequence Synthetic oligonucleotide
ggugaagaguucguaggcatt

SEQ ID NO: 44 - DNA, 627 bp, Mus musculus
atggcatctggacaaggaccaggtcccccgaaggtgggctgcgatgagtccccgtccccttctgaacagcaggttgcccaggacacagaggaggtctttcgaagctacgttttttacctccaccagcaggaacaggagacccaggggcggccgcctgccaaccccgagatggacaacttgcccctggaacccaacagcatcttgggtcaggtgggtcggcagcttgctctcatcggagatgatattaaccggcgctacgacacagagttccagaatttactagaacagcttcagcccacagccgggaatgcctacgaactcttcaccaagatcgcctccagcctatttaagagtggcatcagctggggccgcgtggtggctctcctgggctttggctaccgtctggccctgtacgtctaccagcgtggtttgaccggcttcctgggccaggtgacctgctttttggctgatatcatactgcatcattacatcgccagatggatcgcacagagaggcggttgggtggcagccctgaatttgcgtagagaccccatcctgaccgtaatggtgatttttggtgtggttctgttgggccaattcgtggtacacagattcttcagatcatga

SEQ ID NO: 45 - DNA, 19 bp, Mus musculus
tgcctacgaactcttcacc

SEQ ID NO: 46 - DNA, 21 bp, Artificial Sequence
  Description of Artificial Sequence Synthetic oligonucleotide
uauggagcugcagaggaugtt

SEQ ID NO: 47 - DNA, 21 bp, Artificial Sequence
  Description of Artificial Sequence Synthetic oligonucleotide
cauccucugcagcuccauatt

SEQ ID NO: 48 - DNA, 579 bp, Mus musculus
atggacgggtccggggagcagcttgggagcggcgggcccaccagctctgaacagatcatgaagacaggggcctttttgctacagggtttcatccaggatcgagcagggaggatggctggggagacacctgagctgaccttggagcagccgccccaggatgcgtccaccaagaagctgagcgagtgtctccggcgaattggagatgaactggatagcaatatggagctgcagaggatgattgctgacgtggacacggactccccccgagaggtcttcttccgggtggcagctgacatgtttgctgatggcaacttcaactggggccgcgtggttgccctcttctactttgctagcaaactggtgctcaaggccctgtgcactaaagtgcccgagctgatcagaaccatcatgggctggacactggacttcctccgtgagcggctgcttgtctggatccaagaccagggtggctgggaaggcctcctctcctacttcgggacccccacatggcagacagtgaccatctttgtggctggagtcctcaccgcctcgctcaccatctggaagaagatgggctga

SEQ ID NO: 49 - DNA, 19 bp, Mus musculus
tatggagctgcagaggatg

SEQ ID NO: 50 - DNA, 1491 bp, Homo sapiens
atggacttcagcagaaatctttatgatattggggaacaactggacagtgaagatctggcctccctcaagttcctgagcctggactacattccgcaaaggaagcaagaacccatcaaggatgccttgatgttattccagagactccaggaaaagagaatgttggaggaaagcaatctgtccttcctgaaggagctgctcttccgaattaatagactggatttgctgattacctacctaaacactagaaaggaggagatggaaagggaacttcagacaccaggcagggctcaaatttctgcctacaggttccacttctgccgcatgagctgggctgaagcaaacagccagtgccagacacagtctgtacctttctggcggagggtcgatcatctattaataagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaaggggaggagttgtgtggggtaatgacaatctcggactctccaagagaacaggatagtgaatcacagactttggacaaagtttaccaaatgaaaagcaaacctcggggatactgtctgatcatcaacaatcacaattttgcaaaagcacgggagaaagtgcccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgctgtatcctctcccatggagacaagggcatcatctatggcactgatggacaggaggcccccatctatgagctgacatctcagttcactggtttgaagtgcccttcccttgctggaaaacccaaagtgttttttattcaggcttgtcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattga

SEQ ID NO: 51 - DNA, 23 bp, Artificial Sequence
  Description of Artificial Sequence Synthetic oligonucleotide
aaccucggggauacugucugatt

SEQ ID NO: 52 - DNA, 23 bp, Artificial Sequence
  Description of Artificial Sequence Synthetic oligonucleotide
ucagacaguauccccgagguutt

SEQ ID NO: 53 - DNA, 1251 bp, Homo sapiens
atggacgaagcggatcggcggctcctgcggcggtgccggctgcggctggtggaagagctgcaggtggaccagctctgggacgccctgctgagccgcgagctgttcaggccccatatgatcgaggacatccagcgggcaggctctggatctcggcgggatcaggccaggcagctgatcatagatctggagactcgagggagtcaggctcttcctttgttcatctcctgcttagaggacacaggccaggacatgctggcttcgtttctgcgaactaacaggcaagcagcaaagttgtcgaagccaaccctagaaaaccttaccccagtggtgctcagaccagagattcgcaaaccagaggttctcagaccggaaacacccagaccagtggacattggttctggaggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcataa

SEQ ID NO: 54 - DNA, 834 bp, Homo sapiens
atggagaacactgaaaactcagtggattcaaaatccattaaaaatttggaaccaaagatcatacatggaagcgaatcaatggactctggaatatccctggacaacagttataaaatggattatcctgagatgggtttatgtataataattaataataagaattttcataaaagcactggaatgacatctcggtctggtacagatgtcgatgcagcaaacctcagggaaacattcagaaacttgaaatatgaagtcaggaataaaaatgatcttacacgtgaagaaattgtggaattgatgcgtgatgtttctaaagaagatcacagcaaaaggagcagttttgtttgtgtgcttctgagccatggtgaagaaggaataatttttggaacaaatggacctgttgacctgaaaaaaataacaaactttttcagaggggatcgttgtagaagtctaactggaaaacccaaacttttcattattcaggcctgccgtggtacagaactggactgtggcattgagacagacagtggtgttgatgatgacatggcgtgtcataaaataccagtggaggccgacttcttgtatgcatactccacagcacctggttattattcttggcgaaattcaaaggatggctcctggttcatccagtcgctttgtgccatgctgaaacagtatgccgacaagcttgaatttatgcacattcttacccgggttaaccgaaaggtggcaacagaatttgagtccttttcctttgacgctacttttcatgcaaagaaacagattccatgtattgtttccatgctcacaaaagaactctatttttatcactaa

SEQ ID NO: 55 - DNA, 750 bp, Artificial Sequence
  Description of Artificial Sequence Synthetic polynucleotide
atggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatga

SEQ ID NO: 56 - protein, 249 aa, Artificial Sequence
  Description of Artificial Sequence Synthetic polypeptide
MAYPYDVPDYASLRSTMSQSNRELVVDFLSYKLSQKGYSWSQFSDVEENRTEAPEGTESEMETPSAINGNPSWHLADSPAVNGATAHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 57 - DNA, 6187 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattccaccacactggactagtggatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaagatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaaatgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 58 - DNA, 6452 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccaggatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctactgggctcactcttcagtcggaaatgaagatccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 59 - protein, 349 aa, Artificial Sequence
  Description of Artificial Sequence Synthetic polypeptide
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPGSMAYPYDVPDYASLRSTMSQSNRELVVDFLSYKLSQKGYSWSQFSDVEENRTEAPEGTESEMETPSAINGNPSWHLADSPAVNGATAHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 60 - DNA, 750 bp, Artificial Sequence
  Description of Artificial Sequence Synthetic polynucleotide
atggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtagccattcttcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatga

SEQ ID NO: 61 - protein, 249 aa, Artificial Sequence
  Description of Artificial Sequence Synthetic polypeptide
MAYPYDVPDYASLRSTMSQSNRELVVDFLSYKLSQKGYSWSQFSDVEENRTEAPEGTESEMETPSAINGNPSWHLADSPAVNGATAHSSSLDAREVIPMAAVKQALREAGDEFELRYRRAFSDLTSQLHITPGTAYQSFEQVVNELFRDGVAILRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 62 - protein, 349 aa, Artificial Sequence
  Description of Artificial Sequence Synthetic polypeptide
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPGSMAYPYDVPDYASLRSTMSQSNRELVVDFLSYKLSQKGYSWSQFSDVEENRTEAPEGTESEMETPSAINGNPSWHLADSPAVNGATAHSSSLDAREVIPMAAVKQALREAGDEFELRYRRAFSDLTSQLHITPGTAYQSFEQVVNELFRDGVAILRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 63 - DNA, 6187 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattccaccacactggactagtggatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtagccattcttcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaagatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaaatgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 64 - DNA, 6451 bp, Artificial Sequence
  Description of Artificial Sequence Synthetic polynucleotide
acggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccaggatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtagccattcttcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaagatccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 65 - DNA, 12347 bp, Artificial Sequence
  Description of Artificial Sequence Synthetic polynucleotide
atggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgctagccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattggatcccaaacgggccctctagactcgagcggccgccactgtgctggatatctgcagaattccaccacactggactagtggatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcatggttctactgggctcactcttcagtcggaaatgaagatccgagctcggtaccaagcttaagtttgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgtatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaaactagtgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctagtctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcgcgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggcattctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttctgtctaagcggatgccgggagcagacaagcccgtcagggcgcgtcagcgggtgttggcgggtgtcggggctggcttaactatgcggcatcagagcagattgtactgagagtgcaccatatcgacgctctcccttatgcgactcctgcattaggaagcagcccagtactaggttgaggccgttgagcaccgccgccgcaaggaatggtgcatgcgtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccggaagcttgaattc

SEQ ID NO: 66 - DNA, 12612 bp, Artificial Sequence
  Description of Artificial Sequence Synthetic polynucleotide
atggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgctagccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattggatcccaaacgggccctctagactcgagcggccgccactgtgctggatatctgcagaattcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccaggatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaagatccaagcttaagtttgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgtatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaaactagtgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctagtctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcgcgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggcattctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttctgtctaagcggatgccgggagcagacaagcccgtcagggcgcgtcagcgggtgttggcgggtgtcggggctggcttaactatgcggcatcagagcagattgtactgagagtgcaccatatcgacgctctcccttatgcgactcctgcattaggaagcagcccagtactaggttgaggccgttgagcaccgccgccgcaaggaatggtgcatgcgtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccggaagcttgaattc

SEQ ID NO: 67 - DNA, 12347 bp, Artificial Sequence
  Description of Artificial Sequence Synthetic polynucleotide
atggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgctagccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattggatcccaaacgggccctctagactcgagcggccgccactgtgctggatatctgcagaattccaccacactggactagtggatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtagccattcttcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaagatccgagctcggtaccaagcttaagtttgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgtatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaaactagtgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctagtctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcgcgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggcattctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttctgtctaagcggatgccgggagcagacaagcccgtcagggcgcgtcagcgggtgttggcgggtgtcggggctggcttaactatgcggcatcagagcagattgtactgagagtgcaccatatcgacgctctcccttatgcgactcctgcattaggaagcagcccagtactaggttgaggccgttgagcaccgccgccgcaaggaatggtgcatgcgtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccggaagcttgaattc

SEQ ID NO: 68 - DNA, 12612 bp, Artificial Sequence
  Description of Artificial Sequence Synthetic polynucleotide
atggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgctagccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattggatcccaaacgggccctctagactcgagcggccgccactgtgctggatatctgcagaattcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccaggatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtagccattcttcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaagatccaagcttaagtttgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgtatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaaactagtgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctagtctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcgcgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggcattctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttctgtctaagcggatgccgggagcagacaagcccgtcagggcgcgtcagcgggtgttggcgggtgtcggggctggcttaactatgcggcatcagagcagattgtactgagagtgcaccatatcgacgctctcccttatgcgactcctgcattaggaagcagcccagtactaggttgaggccgttgagcaccgccgccgcaaggaatggtgcatgcgtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagaccggaagcttgaattc

SEQ ID NO: 69 - DNA, 4832 bp, Artificial Sequence
  Description of Artificial Sequence Synthetic polynucleotide
gtcgacttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcggatccagatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaggccagcaaaaggccaggaaccgtaaaaaggccgcgttgctggcgttttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcttttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaatt

SEQ ID NO: 70 - DNA, 4832 bp, Artificial Sequence
  Description of Artificial Sequence Synthetic polynucleotide
gtcgacttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcggatccagatctatggcgtacccatacgatgttccagattacgctagcttgagatctaccatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactgcgcacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtagccattcttcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaggccagcaaaaggccaggaaccgtaaaaaggccgcgttgctggcgttttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcttttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaatt

SEQ ID NO: 71 - DNA, 1499 bp, Artificial Sequence
  Description of Artificial Sequence Synthetic polynucleotide
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagatagatggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtattgtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtcttaatctaa

SEQ ID NO: 72 - protein, 497 aa, Artificial Sequence [sequence omitted]

SEQ ID NO: 73 - DNA, 5575 bp, Artificial Sequence
  Description of Artificial Sequence Synthetic polynucleotide
gtcgacttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaataatcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcggatccatgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagatagatggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtattgtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtcttaatctaaagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcttttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaatt

SEQ ID NO: 74 - DNA, 1395 bp, Artificial Sequence
  Description of Artificial Sequence Synthetic polynucleotide
atggacttcagcagaaatctttatgatattggggaacaactggacagtgaagatctggcctccctcaagttcctgagcctggactacattccgcaaaggaagcaagaacccatcaaggatgccttgatgttattccagagactccaggaaaagagaatgttggaggaaagcaatctgtccttcctgaaggagctgctcttccgaattaatagactggatttgctgattacctacctaaacactagaaaggaggagatggaaagggaacttcagacaccaggcagggctcaaatttctgcctacagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaaggggaggagttgtgtggggtaatgacaatctcggactctccaagagaacaggatagtgaatcacagactttggacaaagtttaccaaatgaaaagcaaacctcggggatactgtctgatcatcaacaatcacaattttgcaaaagcacgggagaaagtgcccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgctgtatcctctcccatggagacaagggcatcatctatggcactgatggacaggaggcccccatctatgagctgacatctcagttcactggtttgaagtgcccttcccttgctggaaaacccaaagtgttttttattcaggcttgtcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattga

SEQ ID NO: 75 - protein, 464 aa, Artificial Sequence
  Description of Artificial Sequence Synthetic polypeptide
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 76 - DNA, 5471 bp, Artificial Sequence
  Description of Artificial Sequence Synthetic polynucleotide
gtcgacttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcatggacttcagcagaaatctttatgatattggggaacaactggacagtgaagatctggcctccctcaagttcctgagcctggactacattccgcaaaggaagcaagaacccatcaaggatgccttgatgttattccagagactccaggaaaagagaatgttggaggaaagcaatctgtccttcctgaaggagctgctcttccgaattaatagactggatttgctgattacctacctaaacactagaaaggaggagatggaaagggaacttcagacaccaggcagggctcaaatttctgcctacagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaaggggaggagttgtgtggggtaatgacaatctcggactctccaagagaacaggatagtgaatcacagactttggacaaagtttaccaaatgaaaagcaaacctcgggatactgtctgatcatcaacaatcacaattttgcaaaagcacgggagaaagtgccccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgctgtatcctctcccatggagacaagggcatcatctatggcactgatggacaggaggcccccatctatgagctgacatctcagttcactggtttgaagtgcccttcccttgctggaaaacccaaagtgttttttattcaggcttgtcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattgaggatccagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcttttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaatt

SEQ ID NO: 77 - DNA, 618 bp, Artificial Sequence
  Description of Artificial Sequence Synthetic polynucleotide
atggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggtaggtgcacttggtgatgtgagtctgggctga

SEQ ID NO: 78 - protein, 205 aa, Artificial Sequence
  Description of Artificial Sequence Synthetic polypeptide
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWVGALGDVSLG

SEQ ID NO: 79 - DNA, 4699 bp, Artificial Sequence
  Description of Artificial Sequence Synthetic polynucleotide
gtcgacttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcggatccagatctatggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggtaggtgcacttggtgatgtgagtctgggctgaagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcttttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaatt

SEQ ID NO: 80 - DNA, 5471 bp, Artificial Sequence
  Description of Artificial Sequence Synthetic polynucleotide
gtcgacttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcggatccatggacttcagcagaaatctttatgatattggggaacaactggacagtgaagatctggcctccctcaagttcctgagcctggactacattccgcaaaggaagcaagaacccatcaaggatgccttgatgttattccagagactccaggaaaagagaatgttggaggaaagcaatctgtccttcctgaaggagctgctcttccgaattaatagactggatttgctgattacctacctaaacactagaaaggaggagatggaaagggaacttcagacaccaggcagggctcaaatttctgcctacagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaaggggaggagttgtgtggggtaatgacaatctcggactctccaagagaacaggatagtgaatcacagactttggacaaagtttaccaaatgaaaagcaaacctcggggatactgtctgatcatcaacaatcacaattttgcaaaagcacgggagaaagtgcccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgctgtatcctctcccatggagacaagggcatcatctatggcactgatggacaggaggcccccatctatgagctgacatctcagttcactggtttgaagtgcccttcccttgctggaaaacccaaagtgttttttattcaggcttctcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattgaagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcttttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaatt

SEQ ID NO: 81 - protein, 464 aa, Artificial Sequence
  Description of Artificial Sequence Synthetic polypeptide
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQASQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 82 - DNA, 5327 bp, Artificial Sequence
  Description of Artificial Sequence Synthetic polynucleotide
gtcgacttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcggatccatggacgaagcggatcggcggctcctgcggcggtgccggctgcggctggtggaagagctgcaggtggaccagctctgggacgccctgctgagccgcgagctgttcaggccccatatgatcgaggacatccagcgggcaggctctggatctcggcgggatcaggccaggcagctgatcatagatctggagactcgagggagtcaggctcttcctttgttcatctcctgcttagaggacacaggccaggacatgctggcttcgtttctgcgaactaacaggcaagcagcaaagttgtcgaagccaaccctagaaaaccttaccccagtggtgctcagaccagagattcgcaaaccagaggttctcagaccggaaacacccagaccagtggacattggttctggaggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctctggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcataaagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcttttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaatt

SEQ ID NO: 83 - protein, 416 aa, Artificial Sequence
  Description of Artificial Sequence Synthetic polypeptide
MDEADRRLLRRCRLRLVEELQVDQLWDALLSRELFRPHMIEDIQRAGSGSRRDQARQLIIDLETRGSQALPLFISCLEDTGQDMLASFLRTNRQAAKLSKPTLENLTPVVLRPEIRKPEVLRPETPRPVDIGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQASGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 84 - DNA, 1819 bp, Artificial Sequence
  Description of Artificial Sequence Synthetic polynucleotide
gaattccgggctggattgagaagccgcaactgtgactctgcatcatgaatactctgtctgaaggaaatggcacctttgccatccatcttttgaagatgctatgtcaaagcaacccttccaaaaatgtatgttattctcctgcgagcatctcctctgctctagctatggttctcttgggtgcaaagggacagacggcagtccagatatctcaggcacttggtttgaataaagaggaaggcatccatcagggtttccagttgcttctcaggaagctgaacaagccagacagaaagtactctcttagagtggccaacaggctctttgcagacaaaacttgtgaagtcctccaaacctttaaggagtcctctcttcacttctatgactcagagatggagcagctctcctttgctgaagaagcagaggtgtccaggcaacacataaacacatgggtctccaaacaaactgaaggtaaaattccagagttgttgtcaggtggctccgtcgattcagaaaccaggctggttctcatcaatgccttatattttaaaggaaagtggcatcaaccatttaacaaagagtacacaatggacatgccctttaaaataaacaaggatgagaaaaggccagtgcagatgatgtgtcgtgaagacacatataacctcgcctatgtgaaggaggtgcaggcgcaagtgctggtgatgccatatgaaggaatggagctgagcttggtggttctgctcccagatgagggtgtggacctcagcaaggtggaaaacaatctcacttttgagaagttaacagcctggatggaagcagattttatgaagagcactgatgttgaggttttccttccaaaatttaaactccaagaggattatgacatggagtctctgtttcagcgcttgggagtggtggatgtcttccaagaggacaaggctgacttatcaggaatgtctccagagagaaacctgtgtgtgtccaagtttgttcaccagagtgtagtggagatcaatgaggaaggcacagaggctgcagcagcctctgccatcatagaattttgctgtgcctcttctgtcccaacattctgtgctgaccaccccttccttttcttcatcaggcacaacaaagcaaacagcatcctgttctgtggcaggttctcatctccataaagacacatatactacacagggagagttctctcttcagtatccctaccactcctacagctctgtcaagatgggcaagtagggggaagtcatgttctaagatgaagacactttccttctctgtcagcctgatcttataatgcctgcattcaactctccctgtcttgaatgcatctatgccctttaccaggttatgtctaatgatgccaaataccttctgctatgctattgattgatagcctagccagtaatttatagccagttagaactgacttgactgtgcaagaatgctataatggagctagagagaaggcacaaacactaggaaaggttgctgtttttgcagaggacacagggacatttcccaccactcacatggctgcttacaacctctggaaattccagtttctgtccatgacttgattcctttctttggcttctactggctccagcatcctgcacatacatgtatcgtcattcagttacacacaaacaagtaaaattttaaaaataaataaaaatttaaagagagagtctaaaattttagtaatggttagataatagctgctattgtgcctttttcaggttttaatgtcattattcttgtgtataaagtcaataatttataggaaaacatcagtgccccggaattc

SEQ ID NO: 85 - protein, 374 aa, Artificial Sequence
  Description of Artificial Sequence Synthetic polypeptide
MNTLSEGNGTFAIHLLKMLCQSNPSKNVCYSPASISSALAMVLLGAKGQTAVQISQALGLNKEEGIHQGFQLLLRKLNKPDRKYSLRVANRLFADKTCEVLQTFKESSLHFYDSEMEQLSFAEEAEVSRQHINTWVSKQTEGKIPELLSGGSVDSETRLVLINALYFKGKWHQPFMKEYTMDMPFKINKDEKRPVQMMCREDTYNLAYVKEVQAQVLVMPYEGMELSLVVLLPDEGVDLSKVENNLTFEKLTAWMEADFMKSTDVEVFLPKFKLQEDYDMESLFQRLGVVDVFQEDKADLSGMSPERNLCVSKFVHQSVVEINEEGTEAAAASAIIEFCCASSVPTFCADHPFLFFIRHNKANSILFCGRFSSP

SEQ ID NO: 86 - DNA, 1125 bp, Artificial Sequence
  Description of Artificial Sequence Synthetic polynucleotide
atgaatactctgtctgaaggaaatggcacctttgccatccatcttttgaagatgctatgtcaaagcaacccttccaaaaatgtatgttattctcctgcgagcatctcctctgctctagctatggttctcttgggtgcaaagggacagacggcagtccagatatctcaggcacttggtttgaataaagaggaaggcatccatcagggtttccagttgcttctcaggaagctgaacaagccagacagaaagtactctcttagagtggccaacaggctctttgcagacaaaacttgtgaagtcctccaaacctttaaggagtcctctcttcacttctatgactcagagatggagcagctctcctttgctgaagaagcagaggtgtccaggcaacacataaacacatgggtctccaaacaaactgaaggtaaaattccagagttgttgtcaggtggctccgtcgattcagaaaccaggctggttctcatcaatgccttatattttaaaggaaagtggcatcaaccatttaacaaagagtacacaatggacatgccctttaaaataaacaaggatgagaaaaggccagtgcagatgatgtgtcgtgaagacacatataacctcgcctatgtgaaggaggtgcaggcgcaagtgctggtgatgccatatgaaggaatggagctgagcttggtggttctgctcccagatgagggtgtggacctcagcaaggtggaaaacaatctcacttttgagaagttaacagcctggatggaagcagattttatgaagagcactgatgttgaggttttccttccaaaatttaaactccaagaggattatgacatggagtctctgtttcagcgcttgggagtggtggatgtcttccaagaggacaaggctgacttatcaggaatgtctccagagagaaacctgtgtgtgtccaagtttgttcaccagagtgtagtggagatcaatgaggaaggcagagaggctgcagcagcctctgccatcatagaattttgctgtgcctcttctgtcccaacattctgtgctgaccaccccttccttttcttcatcaggcacaacaaagcaaacagcatcctgttctgtggcaggttctcatctccataa

SEQ ID NO: 87 - protein, 374 aa, Artificial Sequence
  Description of Artificial Sequence Synthetic polypeptide
MNTLSEGNGTFAIHLLKMLCQSNPSKNVCYSPASISSALAMVLLGAKGQTAVQISQALGLNKEEGIHQGFQLLLRKLNKPDRKYSLRVANRLFADKTCEVLQTFKESSLHFYDSEMEQLSFAEEAEVSRQHINTWVSKQTEGKIPELLSGGSVDSETRLVLINALYFKGKWHQPFNKEYTMDMPFKINKDEKRPVQMMCREDTYNLAYVKEVQAQVLVMPYEGMELSLVVLLPDEGVDLSKVENNLTFEKLTAWMEADFMKSTDVEVFLPKFKLQEDYDMESLFQRLGVVDVFQEDKADLSGMSPERNLCVSKFVHQSVVEINEEGREAAAASAIIEFCCASSVPTFCADHPFLFFIRHNKANSILFCGRFSSP

SEQ ID NO: 88 - DNA, 6539 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattcatgaatactctgtctgaaggaaatggcacctttgccatccatcttttgaagatgctatgtcaaagcaacccttccaaaaatgtatgttattctcctgcgagcatctcctctgctctagctatggttctcttgggtgcaaagggacagacggcagtccagatatctcaggcacttggtttgaataaagaggaaggcatccatcagggtttccagttgcttctcaggaagctgaacaagccagacagaaagtactctcttagagtggccaacaggctctttgcagacaaaacttgtgaagtcctccaaacctttaaggagtcctctcttcacttctatgactcagagatggagcagctctcctttgctgaagaagcagaggtgtccaggcaacacataaacacatgggtctccaaacaaactgaaggtaaaattccagagttgttgtcaggtggctccgtcgattcagaaaccaggctggttctcatcaatgccttatattttaaaggaaagtggcatcaaccatttaacaaagagtacacaatggacatgccctttaaaataaacaaggatgagaaaaggccagtgcagatgatgtgtcgtgaagacacatataacctcgcctatgtgaaggaggtgcaggcgcaagtgctggtgatgccatatgaaggaatggagctgagcttggtggttctgctcccagatgagggtgtggacctcagcaaggtggaaaacaatctcacttttgagaagttaacagcctggatggaagcagattttatgaagagcactgatgttgaggttttccttccaaaatttaaactccaagaggattatgacatggagtctctgtttcagcgcttgggagtggtggatgtcttccaagaggacaaggctgacttatcaggaatgtctccagagagaaacctgtgtgtgtccaagtttgttcaccagagtgtagtggagatcaatgaggaaggcacagaggctgcagcagcctctgccatcatagaattttgctgtgcctcttctgtcccaacattctgtgctgaccaccccttccttttcttcatcaggcacaacaaagcaaacagcatcctgttctgtggcaggttctcatctccataaggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 89 - DNA, 6539 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattcatgaatactctgtctgaaggaaatggcacctttgccatccatcttttgaagatgctatgtcaaagcaacccttccaaaaatgtatgttattctcctgcgagcatctcctctgctctagctatggttctcttgggtgcaaagggacagacggcagtccagatatctcaggcacttggtttgaataaagaggaaggcatccatcagggtttccagttgcttctcaggaagctgaacaagccagacagaaagtactctcttagagtggccaacaggctctttgcagacaaaacttgtgaagtcctccaaacctttaaggagtcctctcttcacttctatgactcagagatggagcagctctcctttgctgaagaagcagaggtgtccaggcaacacataaacacatgggtctccaaacaaactgaaggtaaaattccagagttgttgtcaggtggctccgtcgattcagaaaccaggctggttctcatcaatgccttatattttaaaggaaagtggcatcaaccatttaacaaagagtacacaatggacatgccctttaaaataaacaaggatgagaaaaggccagtgcagatgatgtgtcgtgaagacacatataacctcgcctatgtgaaggaggtgcaggcgcaagtgctggtgatgccatatgaaggaatggagctgagcttggtggttctgctcccagatgagggtgtggacctcagcaaggtggaaaacaatctcacttttgagaagttaacagcctggatggaagcagattttatgaagagcactgatgttgaggttttccttccaaaatttaaactccaagaggattatgacatggagtctctgtttcagcgcttgggagtggtggatgtcttccaagaggacaaggctgacttatcaggaatgtctccagagagaaacctgtgtgtgtccaagtttgttcaccagagtgtagtggagatcaatgaggaaggcagagaggctgcagcagcctctgccatcatagaattttgctgtgcctcttctgtcccaacattctgtgctgaccaccccttccttttcttcatcaggcacaacaaagcaaacagcatcctgttctgtggcaggttctcatctccataaggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 90 - DNA, 810 bp, Artificial Sequence
  Description of Artificial Sequence Synthetic polynucleotide
atggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttgccaagttcgtggctgcctggaccctgaaggctgccgctgccctgccccaggggcccatgcagaatgccaccaagtatggcaacatgacagaggaccatgtgatgcacctgctccagaatgctgaccccctgaaggtgtacccgccactgaaggggagcttcccggagaacctgagacaccttaagaacaccatggagaccatagactggaaggtctttgagagctggatgcaccattggctcctgtttgaaatgagcaggcactccttggagcaaaagcccactgacgctccaccgaaagtactgaccaagtgccaggaagaggtcagccacatccctgctgtccacccgggttcattcaggcccaagtgcgacgagaacggcaactatctgccactccagtgctatgggagcatcggctactgctggtgtgtcttccccaacggcacggaggtccccaacaccagaagccgcgggcaccataactgcagtgagtcactggaactggaggacccgtcttctgggctgggtgtgaccaagcaggatctgggcccagtccccatgtga

SEQ ID NO: 91 - protein, 269 aa, Artificial Sequence
  Description of Artificial Sequence Synthetic polypeptide
MDDQRDLISNNEQLPMLGRRPGAPESKCSRGALYTGFSILVTLLLAGQATTAYFLYQQQGRLDKLTVTSQNLQLENLRMKLAKFVAAWTLKAAAALPQGPMQNATKYGNMTEDHVMHLLQNADPLKVYPPLKGSFPENLRHLKNTMETIDWKVFESWMHHWLLFEMSRHSLEQKPTDAPPKVLTKCQEEVSHIPAVHPGSFRPKCDENGNYLPLQCYGSIGYCWCVFPNGTEVPNTRSRGHHNCSESLELEDPSSGLGVTKQDLGPVPM

SEQ ID NO: 92 - protein, 17 aa, Homo sapiens
KPVSQMRMATPLLMRPM

SEQ ID NO: 93 - protein, 13 aa, Pan sp.
AKFVAAWTLKAAA

SEQ ID NO: 94 - DNA, 3392 bp, Homo sapiens
atgcgttgcctggctccacgccctgctgggtcctacctgtcagagccccaaggcagctcacagtgtgccaccatggagttggggcccctagaaggtggctacctggagcttcttaacagcgatgctgacccctgtgcctctaccacttctatgaccagatggacctggctggagaagaagagattgagctctactcagaacccgacacagacaccatcaactgcgaccagttcagcaggctgttgtgtgacatggaaggtgatgaagagaccagggaggcttatgccaatatcgcggaactggaccagtatgtcttccaggactcccagctggagggcctgagcaaggacattttcaagcacataggaccagatgaagtgatcggtgagagtatggagatgccagcagaagttgggcagaaaagtcagaaaagacccttcccagaggagcttccggcagacctgaagcactggaagccagctgagccccccactgtggtgactggcagtctcctagtgggaccagtgagcgactgctccaccctgccctgcctgccactgcctgcgctgttcaaccaggagccagcctccggccagatgcgcctggagaaaaccgaccagattcccatgcctttctccagttcctcgttgagctgcctgaatctccctgagggacccatccagtttgtccccaccatctccactctgccccatgggctctggcaaatctctgaggctggaacaggggtctccagtatattcatctaccatggtgaggtgccccaggccagccaagtaccccctcccagtggattcactgtccacggcctcccaacatctccagaccggccaggctccaccagccccttcgctccatcagccactgacctgcccagcatgcctgaacctgccctgacctcccgagcaaacatgacagagcacaagacgtcccccacccaatgcccggcagctggagaggtctccaacaagcttccaaaatggcctgagccggtggagcagttctaccgctcactgcaggacacgtatggtgccgagcccgcaggcccggatggcatcctagtggaggtggatctggtgcaggccaggctggagaggagcagcagcaagagcctggagcgggaactggccaccccggactgggcagaacggcagctggcccaaggaggcctggctgaggtgctgttggctgccaaggagcaccggcggccgcgtgagacacgagtgattgctgtgctgggcaaagctggtcagggcaagagctattgggctggggcagtgagccgggcctgggcttgtggccggcttccccagtacgactttgtcttctctgtcccctgccattgcttgaaccgtccgggggatgcctatggcctgcaggatctgctcttctccctgggcccacagccactcgtggcggccgatgaggttttcagccacatcttgaagagacctgaccgcgttctgctcatcctagacggcttcgaggagctggaagcgcaagatggcttcctgcacagcacgtgcggaccggcaccggcggagccctgctccctccgggggctgctggccggccttttccagaagaagctgctccgaggttgcaccctcctcctcacagcccggccccggggccgcctggtccagagcctgagcaaggccgacgccctatttgagctgtccggcttctccatggagcaggcccaggcatacgtgatgcgctactttgagagctcagggatgacagagcaccaagacagagccctgacgctcctccgggaccggccacttcttctcagtcacagccacagccctactttgtgccgggcagtgtgccagctctcagaggccctgctggagcttggggaggacgccaagctgccctccacgctcacgggactctatgtcggcctgctgggccgtgcagccctcgacagcccccccggggccctggcagagctggccaagctggcctgggagctgggccgcagacatcaaagtaccctacaggaggaccagttcccatccgcagacgtgaggacctgggcgatggccaaaggcttagtccaacacccaccgcgggccgcagagtccgagctggccttccccagcttcctcctgcaatgcttcctgggggccctgtggctggctctgagtggcgaaatcaaggacaaggagctcccgcagtacctagcattgaccccaaggaagaagaggccctatgacaactggctggagggcgtgccacgctttctggctgggctgatcttccagcctcccgcccgctgcctgggagccatactcgggccatcggcggctgcctcggtggacaggaagcagaaggtgcttgcgaggtacctgaagcggctgcagccggggacactgcgggcgcggcagctgctggagctgctgcactgcgcccacgaggccgaggaggctggaatttggcagcacgtggtacaggagctccccggccgcctctcttttctgggcacccgcctcacgcctcctgatgcacatgtactgggcaaggccttggaggcggcgggccaagacttctccctggacctccgcagcactggcatttgcccctctggattggggagcctcgtgggactcagctgtgtcacccgtttcagggctgccttgagcgacacggtggcgctgtgggagtccctgcagcagcatggggagaccaagctacttcaggcagcagaggagaagttcaccatcgagcctttcaaagccaagtccctgaaggatgtggaagacctgggaaagcttgtgcagactcagaggacgagaagttcctcggaagacacagctggggagctccctgctgttcgggacctaaagaaactggagtttgcgctgggccctgtctcaggcccccaggctttccccaaactggtgcggatcctcacggccttttcctccctgcagcatctggacctggatgcgctgagtgagaacaagatcggggacgagggtgtctcgcagctctcagccaccttcccccagctgaagtccttggaaaccctcaatctgtcccagaacaacatcactgacctgggtgcctacaaactcgccgaggccctgccttcgctcgctgcatccctgctcaggctaagcttgtacaataactgcatctgcgacgtgggagccgagagcttggctcgtgtgcttccggacatggtgtccctccgggtgatggacgtccagtacaacaagttcacggctgccggggcccagcagctcgctgccagccttcggaggtgtcctcatgtggagacgctggcgatgtggacgcccaccatcccattcagtgtccaggaacacctgcaacaacaggattcacggatcagcctgagatga

SEQ ID NO: 95 - protein, 1130 aa, Homo sapiens
MRCLAPRPAGSYLSEPQGSSQCATMELGPLEGGYLELLNSDADPLCLYHFYDQMDLAGEEEIELYSEPDTDTINCDQFSRLLCDMEGDEETREAYANIAELDQYVFQDSQLEGLSKDIFKHIGPDEVIGESMEMPAEVGQKSQKRPFPEELPADLKHWKPAEPPTVVTGSLLVGPVSDCSTLPCLPLPALFNQEPASGQMRLEKTDQIPMPFSSSSLSCLNLPEGPIQFVPTISTLPHGLWQISEAGTGVSSIFIYHGEVPQASQVPPPSGFTVHGLPTSPDRPGSTSPFAPSATDLPSMPEPALTSRANMTEHKTSPTQCPAAGEVSNKLPKWPEPVEQFYRSLQDTYGAEPAGPDGILVEVDLVQARLERSSSKSLERELATPDWAERQLAQGGLAEVLLAAKEHRRPRETRVIAVLGKAGQGKSYWAGAVSRAWACGRLPQYDFVFSVPCHCLNRPGDAYGLQDLLFSLGPQPLVAADEVFSHILKRPDRVLLILDGFEELEAQDGFLHSTCGPAPAEPCSLRGLLAGLFQKKLLRGCTLLLTARPRGRLVQSLSKADALFELSGFSMEQAQAYVMRYFESSGMTEHQDRALTLLRDRPLLLSHSHSPTLCRAVCQLSEALLELGEDAKLPSTLTGLYVGLLGRAALDSPPGALAELAKLAWELGRRHQSTLQEDQFPSADVRTWAMAKGLVQHPPRAAESELAFPSFLLQCFLGALWLALSGEIKDKELPQYLALTPRKKRPYDNWLEGVPRFLAGLIFQPPARCLGALLGPSAAASVDRKQKVLARYLKRLQPGTLRARQLLELLHCAHEAEEAGIWQHVVQELPGRLSFLGTRLTPPDAHVLGKALEAAGQDFSLDLRSTGICPSGLGSLVGLSCVTRFRAALSDTVALWESLQQHGETKLLQAAEEKFTIEPFKAKSLKDVEDLGKLVQTQRTRSSSEDTAGELPAVRDLKKLEFALGPVSGPQAFPKLVRILTAFSSLQHLDLDALSENKIGDEGVSQLSATFPQLKSLETLNLSQNNITDLGAYKLAEALPSLAASLLRLSLYNNCICDVGAESLARVLPDMVSLRVMDVQYNKFTAAGAQQLAASLRRCPHVETLAMWTPTIPFSVQEHLQQQDSRISLR

SEQ ID NO: 96 - DNA, 1518 bp, Human papillomavirus
atgagcctgtggctgcccagcgaggccaccgtgtacctgccccccgtgcccgtgagcaaggtggtgagcaccgacgagtacgtggccaggaccaacatctactaccacgccggcaccagcaggctgctggccgtgggccacccctacttccccatcaagaagcccaacaacaacaagatcctggtgcccaaggtgagcggcctgcagtacagggtgttcaggatccacctgcccgaccccaacaagttcggcttccccgacaccagcttctacaaccccgacacccagaggctggtgtgggcctgcgtgggcgtggaggtgggcaggggccagcccctgggcgtgggcatcagcggccaccccctgctgaacaagctggacgacaccgagaacgccagcgcctacgccgccaacgccggcgtggacaacagggagtgcatcagcatggactacaagcagacccagctgtgcctgatcggctgcaagccccccatcggcgagcactggggcaagggcagcccctgcaccaacgtggccgtgaaccccggcgactgcccccccctggagctgatcaacaccgtgatccaggacggcgacatggtggacaccggcttcggcgccatggacttcaccaccctgcaggccaacaagagcgaggtgcccctggacatctgcaccagcatctgcaagtaccccgactacatcaagatggtgagcgagccctacggcgacagcctgttcttctacctgaggagggagcagatgttcgtgaggcacctgttcaacagggccggcgccgtgggcgagaacgtgcccgacgacctgtacatcaagggcagcggcagcaccgccaacctggccagcagcaactacttccccacccccagcggcagcatggtgaccagcgacgcccagatcttcaacaagccctactggctgcagagggcccagggccacaacaacggcatctgctggggcaaccagctgttcgtgaccgtggtggacaccaccaggagcaccaacatgagcctgtgcgccgccatcagcaccagcgagaccacctacaagaacaccaacttcaaggagtacctgaggcacggcgaggagtacgacctgcagttcatcttccagctgtgcaagatcaccctgaccgccgacgtgatgacctacatccacagcatgaacagcaccatcctggaggactggaacttcggcctgcagcccccccccggcggcaccctggaggacacctacaggttcgtgaccagccaggccatcgcctgccagaagcacaccccccccgcccccaaggaggaccccctgaagaagtacaccttctgggaggtgaacctgaaggagaagttcagcgccgacctggaccagttccccctgggcaggaagttcctgctgcaggccggcctgaaggccaagcccaagttcaccctgggcaagaggaaggccacccccaccaccagcagcaccagcaccaccgccaagaggaagaagaggaagctgtga

SEQ ID NO: 97 - protein, 505 aa, Human papillomavirus
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKFTLGKRKATPTTSSTSTTAKRKKRKL

SEQ ID NO: 98 - DNA, 1707 bp, Human papillomavirus
atgtgcctgtatacacgggtcctgatattacattaccatctactacctctgtatggcccattgtatcacccacggcccctgcctctacacagtatattggtatacatggtacacattattatttgtggccattatattattttattcctaagaaacgtaaacgtgttccctatttttttgcagatggctttgtggcggcctagtgacaataccgtatatcttccacctccttctgtggcaagagttgtaaataccgatgattatgtgactcccacaagcatattttatcatgctggcagctctagattattaactgttggtaatccatattttagggttcctgcaggtggtggcaataagcaggatattcctaaggtttctgcataccaatatagagtatttagggtgcagttacctgacccaaataaatttggtttacctgatactagtatttataatcctgaaacacaacgtttagtgtgggcctgtgctggagtggaaattggccgtggtcagcctttaggtgttggccttagtgggcatccattttataataaattagatgacactgaaagttcccatgccgccacgtctaatgtttctgaggacgttagggacaatgtgtctgtagattataagcagacacagttatgtattttgggctgtgcccctgctattggggaacactgggctaaaggcactgcttgtaaatcgcgtcctttatcacagggcgattgcccccctttagaacttaaaaacacagttttggaagatggtgatatggtagatactggatatggtgccatggactttagtacattgcaagatactaaatgtgaggtaccattggatatttgtcagtctatttgtaaatatcctgattatttacaaatgtctgcagatccttatggggattccatgtttttttgcttacggcgtgagcagctttttgctaggcatttttggaatagagcaggtactatgggtgacactgtgcctcaatccttatatattaaaggcacaggtatgcctgcttcacctggcagctgtgtgtattctccctctccaagtggctctattgttacctctgactcccagttgtttaataaaccatattggttacataaggcacagggtcataacaatggtgtttgctggcataatcaattatttgttactgtggtagataccactcccagtaccaatttaacaatatgtgcttctacacagtctcctgtacctgggcaatatgatgctaccaaatttaagcagtatagcagacatgttgaggaatatgatttgcagtttatttttcagttgtgtactattactttaactgcagatgttatgtcctatattcatagtatgaatagcagtattttagaggattggaactttggtgttcccccccccccaactactagtttggtggatacatatcgttttgtacaatctgttgctattacctgtcaaaaggatgctgcaccggctgaaaataaggatccctatgataagttaaagttttggaatgtggatttaaaggaaaagttttctttagacttagatcaatatccccttggacgtaaatttttggttcaggctggattgcgtcgcaagcccaccataggccctcgcaaacgttctgctccatctgccactacgtcttctaaacctgccaagcgtgtgcgtgtacgtgccaggaagtaa

SEQ ID NO: 99 - protein, 568 aa, Human papillomavirus
MCLYTRVLILHYHLLPLYGPLYHPRPLPLHSILVYMVHIIICGHYIILFLRNVNVFPIFLQMALWRPSDNTVYLPPPSVARVVNTDDYVTPTSIFYHAGSSRLLTVGNPYFRVPAGGGNKQDIPKVSAYQYRVFRVQLPDPNKFGLPDTSIYNPETQRLVWACAGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPQSLYIKGTGMPASPGSCVYSPSPSGSIVTSDSQLFNKPYWLHKAQGHNNGVCWHNQLFVTVVDTTPSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLVQAGLRRKPTIGPRKRSAPSATTSSKPAKRVRVRARK

SEQ ID NO: 100 - DNA, 1533 bp, Human papillomavirus
atgtcttgtggcctaaacgacgtaaacgtgtccactatttctttgcagatggctttgtggcggcctaatgaaagcaaggtatacctacctccaacacctgtttcaaaggtgatcagtacggatgtctatgtcacgcggactaatgtgtattaccatggtggcagttctaggcttctcactgtgggtcatccatattactctataaagaagagtaataataaggtggctgtgcccaaggtatctgggtaccaatatcgtgtatttcacgtgaagttgccagatccaaataagtttggcctgcccgatgctgatttgtatgatccagatacccagagacttctgtgggcgtgcgtgggagtagaggtgggccgtgggcagcctttgggtgtgggtgtgtctggtcacccatattacaatagactggatgacactgaaaatgcacacacacctgatacagctgatgatggcagggaaaacatttctatggattataaacagacacagctgttcattctgggctgcaaaccccctattggtgagcactggtctaagggtaccacctgtaatgggtcttctgctgctggtgactgcccgcccctccaatttactaacacaactattgaggacggggatatggttgaaacagggttcggtgccttggattttgccactctgcagtcaaataagtcagatgttcctttggatatttgtaccaatacctgtaaatatcctgattatctgaagatggctgcagagccttatggtgattctatgttcttctcgctgcgtagggaacaaatgttcactcgtcattttttcaatctgggtggtaagatgggtgacaccatcccggatgagttatacattaaaagtacctcagttccaactccaggcagtcatgtttatacttccactcctagtggctctatggtgtcctctgaacaacagttgtttaataagccttactggctacggagggcccaagggcacaacaatggtatgtgctggggcaatagggtctttctgactgtggtggacaccacacgtagcactaatgtatctctgtgtgccactgaggcgtctgatactaattataaggctaccaattttaaggaatatctcaggcatatggaggaatatgatttgcagttcatcttccaactgtgcaagataacccttactcctgaaattatggcctatatacataatatggatccccagttgttagaggattggaacttcggtgtaccccctccgccgtctgccagtttacaggatacctatagatatttgcagtcccaggctattacatgtcaaaaacctacacctcctaagacccctaccgatccctatgcctccctgaccttttgggatgtggatctcagtgaaagtttttccatggatctggaccaatttcccttgggtcgcaagtttttgctgcagcggggggctatgcctaccgtgtctcgcaagcgcgccgctgtttcggggaccacgccgcccactagtaaacgaaaacgggtaaggcgttag

SEQ ID NO: 101 - protein, 510 aa, Human papillomavirus
MSCGLNDVNVSTISLQMALWRPNESKVYLPPTPVSKVISTDVYVTRTNVYYHGGSSRLLTVGHPYYSIKKSNNKVAVPKVSGYQYRVFHVKLPDPNKFGLPDADLYDPDTQRLLWACVGVEVGRGQPLGVGVSGHPYYNRLDDTENAHTPDTADDGRENISMDYKQTQLFILGCKPPIGEHWSKGTTCNGSSAAGDCPPLQFTNTTIEDGDMVETGFGALDFATLQSNKSDVPLDICTNTCKYPDYLKMAAEPYGDSMFFSLRREQMFTRHFFNLGGKMGDTIPDELYIKSTSVPTPGSHVYTSTPSGSMVSSEQQLFNKPYWLRRAQGHNNGMCWGNRVFLTVVDTTRSTNVSLCATEASDTNYKATNFKEYLRHMEEYDLQFIFQLCKITLTPEIMAYIHNMDPQLLEDWNFGVPPPPSASLQDTYRYLQSQAITCQKPTPPKTPTDPYASLTFWDVDLSESFSMDLDQFPLGRKFLLQRGAMPTVSRKRAAVSGTTPPTSKRKRVRR

SEQ ID NO: 102 - DNA, 1422 bp, Human papillomavirus
atgaggcacaagaggagcgccaagaggaccaagagggccagcgccacccagctgtacaagacctgcaagcaggccggcacctgcccccccgacatcatccccaaggtggagggcaagaccatcgccgaccagatcctgcagtacggcagcatgggcgtgttcttcggcggcctgggcatcggcaccggcagcggcaccggcggcaggaccggctacatccccctgggcaccaggccccccaccgccaccgacaccctggcccccgtgaggccccccctgaccgtggaccccgtgggccccagcgaccccagcatcgtgagcctggtggaggagaccagcttcatcgacgccggcgcccccaccagcgtgcccagcatcccccccgacgtgagcggcttcagcatcaccaccagcaccgacaccacccccgccatcctggacatcaacaacaccgtgaccaccgtgaccacccacaacaaccccaccttcaccgaccccagcgtgctgcagccccccacccccgccgagaccggcggccacttcaccctgagcagcagcaccatcagcacccacaactacgaggagatccccatggacaccttcatcgtgagcaccaaccccaacaccgtgaccagcagcacccccatccccggcagcaggcccgtggccaggctgggcctgtacagcaggaccacccagcaggtgaaggtggtggaccccgccttcgtgaccacccccaccaagctgatcacctacgacaaccccgcctacgagggcatcgacgtggacaacaccctgtacttcagcagcaacgacaacagcatcaacatcgcccccgaccccgacttcctggacatcgtggccctgcacaggcccgccctgaccagcaggaggaccggcatcaggtacagcaggatcggcaacaagcagaccctgaggaccaggagcggcaagagcatcggcgccaaggtgcactactactacgacctgagcaccatcgaccccgccgaggagatcgagctgcagaccatcacccccagcacctacaccaccaccagccacgccgccagccccaccagcatcaacaacggcctgtacgacatctacgccgacgacttcatcaccgacaccagcaccacccccgtgcccagcgtgcccagcaccagcctgagcggctacatccccgccaacaccaccatccccttcggtggcgcctacaacatccccctggtgagcggccccgacatccccatcaacatcaccgaccaggcccccagcctgatccccatcgtgcccggcagcccccagtacaccatcatcgccgacgccggcgacttctacctgcaccccagctactacatgctgaggaagaggaggaagaggctgccctacttcttcagcgacgtgagcctggccgcctga

SEQ ID NO: 103 - protein, 473 aa, Human papillomavirus
MRHKRSAKRTKRASATQLYKTCKQAGTCPPDIIPKVEGKTIADQILQYGSMGVFFGGLGIGTGSGTGGRTGYIPLGTRPPTATDTLAPVRPPLTVDPVGPSDPSIVSLVEETSFIDAGAPTSVPSIPPDVSGFSITTSTDTTPAILDINNTVTTVTTHNNPTFTDPSVLQPPTPAETGGHFTLSSSTISTHNYEEIPMDTFIVSTNPNTVTSSTPIPGSRPVARLGLYSRTTQQVKVVDPAFVTTPTKLITYDNPAYEGIDVDNTLYFSSNDNSINIAPDPDFLDIVALHRPALTSRRTGIRYSRIGNKQTLRTRSGKSIGAKVHYYYDLSTIDPAEEIELQTITPSTYTTTSHAASPTSINNGLYDIYADDFITDTSTTPVPSVPSTSLSGYIPANTTIPFGGAYNIPLVSGPDIPINITDQAPSLIPIVPGSPQYTIIADAGDFYLHPSYYMLRKRRKRLPYFFSDVSLAA

SEQ ID NO: 104 - DNA, 1389 bp, Human papillomavirus
atggtatcccaccgtgccgcacgacgcaaacgggcttcggtaactgacttatataaaacatgtaaacaatctggtacatgtccacctgatgttgttcctaaggtggagggcaccacgttagcagataaaatattgcaatggtcaagccttggtatatttttgggtggacttggcataggtactggcagtggtacagggggtcgtacagggtacattccattgggtgggcgttccaatacagtggtggatgttggtcctacacgtcccccagtggttattgaacctgtgggccccacagacccatctattgttacattaatagaggactccagtgtggttacatcaggtgcacctaggcctacgtttactggcacgtctgggtttgatataacatctgcgggtacaactacacctgcggttttggatatcacaccttcgtctacctctgtgtctatttccacaaccaattttaccaatcctgcattttctgatccgtccattattgaagttccacaaactggggaggtggcaggtaatgtatttgttggtacccctacatctggaacacatgggtatgaggaaatacctttacaaacatttgcttcttctggtacgggggaggaacccattagtagtaccccattgcctactgtgcggcgtgtagcaggtccccgcctttacagtagggcctaccaacaagtgtcagtggctaaccctgagtttcttacacgtccatcctctttaattacatatgacaacccggcctttgagcctgtggacactacattaacatttgatcctcgtagtgatgttcctgattcagattttatggatattatccgtctacataggcctgctttaacatccaggcgtgggactgttcgctttagtagattaggtcaacgggcaactatgtttacccgcagcggtacacaaataggtgctagggttcacttttatcatgatataagtcctattgcaccttccccagaatatattgaactgcagcctttagtatctgccacggaggacaatgacttgtttgatatatatgcagatgacatggaccctgcagtgcctgtaccatcgcgttctactacctcctttgcattttttaaatattcgcccactatatcttctgcctcttcctatagtaatgtaacggtccctttaacctcctcttgggatgtgcctgtatacacgggtcctgatattacattaccatctactacctctgtatggcccattgtatcacccacggcccctgcctctacacagtatattggtatacatggtacacattattatttgtggccattatattattttattcctaagaaacgtaaacgtgttccctatttttttgcagatggctttgtggcggcctag

SEQ ID NO: 105 - protein, 462 aa, Human papillomavirus
MVSHRAARRKRASVTDLYKTCKQSGTCPPDVVPKVEGTTLADKILQWSSLGIFLGGLGIGTGSGTGGRTGYIPLGGRSNTVVDVGPTRPPVVIEPVGPTDPSIVTLIEDSSVVTSGAPRPTFTGTSGFDITSAGTTTPAVLDITPSSTSVSISTTNFTNPAFSDPSIIEVPQTGEVAGNVFVGTPTSGTHGYEEIPLQTFASSGTGEEPISSTPLPTVRRVAGPRLYSRAYQQVSVANPEFLTRPSSLITYDNPAFEPVDTTLTFDPRSDVPDSDFMDIIRLHRPALTSRRGTVRFSRLGQRATMFTRSGTQIGARVHFYHDISPIAPSPEYIELQPLVSATEDNDLFDIYADDMDPAVPVPSRSTTSFAFFKYSPTISSASSYSNVTVPLTSSWDVPVYTGPDITLPSTTSVWPIVSPTAPASTQYIGIHGTHYYLWPLYYFIPKKRKRVPYFFADGFVAA

SEQ ID NO: 106 - DNA, 1575 bp, Human papillomavirus
atgtctgttggtgattcttatcctaatcgcctttttattgttgatgttttatgtccgtttgttaaaccacacctaacacccccacttttttatattgttttgatacattttcattttgatacatttgtgttttttttgtatttgctgcgttttaataaacgtgcaaccatgtctatacgtgccaagcgtcgaaagcgcgcctcccccacagacctctatcgtacctgcaagcaggcaggtacctgccccccagacattatcccaagagtggaacagaacactttagcagataaaatccttaagtggggcagtttaggtgtgttttttgggggtctaggtataggcaccggcagcggcacaggggggcgtactgggtacattcctgtaggttcgcgacccaccactgtagttgacattggtccaacgcccaggccgcctgttatcattgaacctgtgggggcctctgaaccctctattgtcactttggtggaggactctagcatcattaacgcaggagcgtcacatcccacctttactggtactggtggcttcgaagtgacaacctccaccgttacagaccccgccgtcttggatatcaccccctcaggtaccagtgtgcaggtcagcagcagtagctttcttaacccactatacactgagccagctattgtggaggctccccaaacaggggaagtatctggccatgtacttgttagtacagccacctcagggtctcatggctatgaggaaataccaatgcagacgtttgccacgtcggggggcagcggtacagagcctatcagtagcacacccctccctggcgtgcggagagttgccggaccccgcctgtacagtagagccaatcagcaagtgcaagtcagggatcctgcgtttcttgcaaggcctgctgatctagtaacatttgacaatcctgtgtatgacccagaggaaactataatatttcagcatccagacttgcatgagccaccggatcctgattttttggacatagtggcgttgcatcgtcccgccctcacgtccagaaggggtactgtccgttttagtaggttgggacgcagggctacactccgcacccgtagtggtaaacaaattggggcacgggtgcacttctatcatgatattagccctataggtactgaggagttggagatggagccactgttgcccccagcttctactgataacacagatatgttatatgatgtttatgctgattcggatgtccttcagccattgcttgatgagttacccgccgcccctcgcggttcactctctctggctgacactgctgtgtctgccacctccgcatctacactacgggggtccactactgtccctttatcaagtggtattgatgtgcctgtgtacaccggtcctgacattgaaccacccaatgttcctggcatgggacctctgattcctgtggctccatccttaccatcgtctgtgtacatatttgggggagattattatttgatgccaagttatgtcttgtggcctaaacgacgtaaacgtgtccactatttctttgcagatggctttgtggcggcctaa

SEQ ID NO: 107 - protein, 524 aa, Human papillomavirus
MSVGDSYPNRLFIVDVLCPFVKPHLTPPLFYIVLIHFHFDTFVFFLYLLRFNKRATMSIRAKRRKRASPTDLYRTCKQAGTCPPDIIPRVEQNTLADKILKWGSLGVFFGGLGIGTGSGTGGRTGYIPVGSRPTTVVDIGPTPRPPVIIEPVGASEPSIVTLVEDSSIINAGASHPTFTGTGGFEVTTSTVTDPAVLDITPSGTSVQVSSSSFLNPLYTEPAIVEAPQTGEVSGHVLVSTATSGSHGYEEIPMQTFATSGGSGTEPISSTPLPGVRRVAGPRLYSRANQQVQVRDPAFLARPADLVTFDNPVYDPEETIIFQHPDLHEPPDPDFLDIVALHRPALTSRRGTVRFSRLGRRATLRTRSGKQIGARVHFYHDISPIGTEELEMEPLLPPASTDNTDMLYDVYADSDVLQPLLDELPAAPRGSLSLADTAVSATSASTLRGSTTVPLSSGIDVPVYTGPDIEPPNVPGMGPLIPVAPSLPSSVYIFGGDYYLMPSYVLWPKRRKRVHYFFADGFVAA

SEQ ID NO: 108 - DNA, 2385 bp, Homo sapiens
atggagctgaggccctggttgctatgggtggtagcagcaacaggaaccttggtcctgctagcagctgatgctcagggccagaaggtcttcaccaacacgtgggctgtgcgcatccctggaggcccagcggtggccaacagtgtggcacggaagcatgggttcctcaacctgggccagatcttcggggactattaccacttctggcatcgaggagtgacgaagcggtccctgtcgcctcaccgcccgcggcacagccggctgcagagggagcctcaagtacagtggctggaacagcaggtggcaaagcgacggactaaacgggacgtgtaccaggagcccacagaccccaagtttcctcagcagtggtacctgtctggtgtcactcagcgggacctgaatgtgaaggcggcctgggcgcagggctacacagggcacggcattgtggtctccattctggacgatggcatcgagaagaaccacccggacttggcaggcaattatgatcctggggccagttttgatgtcaatgaccaggaccctgacccccagcctcggtacacacagatgaatgacaacaggcacggcacacggtgtgcgggggaagtggctgcggtggccaacaacggtgtctgtggtgtaggtgtggcctacaacgcccgcattggaggggtgcgcatgctggatggcgaggtgacagatgcagtggaggcacgctcgctgggcctgaaccccaaccacatccacatctacagtgccagctggggccccgaggatgacggcaagacagtggatgggccagcccgcctcgccgaggaggccttcttccgtggggttagccagggccgaggggggctgggctccatctttgtctgggcctcggggaacgggggccgggaacatgacagctgcaactgcgacggctacaccaacagtatctacacgctgtccatcagcagcgccacgcagtttggcaacgtgccgtggtacagcgaggcctgctcgtccacactggccacgacctacagcagtggcaaccagaatgagaagcagatcgtgacgactgacttgcggcagaagtgcacggagtctcacacgggcacctcagcctctgcccccttagcagccggcatcattgctctcaccctggaggccaataagaacctcacatggcgggacatgcaacacctggtggtacagacctcgaagccagcccacctcaatgccaacgactgggccaccaatggtgtgggccggaaagtgagccactcatatggctacgggcttttggacgcaggcgccatggtggccctggcccagaattggaccacagtggccccccagcggaagtgcatcatcgacatcctcaccgagcccaaagacatcgggaaacggctcgaggtgcggaagaccgtgaccgcgtgcctgggcgagcccaaccacatcactcggctggagcacgctcaggcgcggctcaccctgtcctataatcgccgtggcgacctggccatccacctggtcagccccatgggcacccgctccaccctgctggcagccaggccacatgactactccgcagatgggtttaatgactgggccttcatgacaactcattcctgggatgaggatccctctggcgagtgggtcctagagattgaaaacaccagcgaagccaacaactatgggacgctgaccaagttcaccctcgtactctatggcaccgcccctgaggggctgcccgtacctccagaaagcagtggctgcaagaccctcacgtccagtcaggcctgtgtggtgtgcgaggaaggcttctccctgcaccagaagagctgtgtccagcactgccctccagggttcgccccccaagtcctcgatacgcactatagcaccgagaatgacgtggagaccatccgggccagcgtctgcgccccctgccacgcctcatgtgccacatgccaggggccggccctgacagactgcctcagctgccccagccacgcctccttggaccctgtggagcagacttgctcccggcaaagccagagcagccgagagtccccgccacagcagcagccacctcggctgcccccggaggtggaggcggggcaacggctgcgggcagggctgctgccctcacacctgcctgaggtggtggccggcctcagctgcgccttcatcgtgctggtcttcgtcactgtcttcctggtcctgcagctgcgctctggctttagttttcggggggtgaaggtgtacaccatggaccgtggcctcatctcctacaaggggctgccccctgaagcctggcaggaggagtgcccgtctgactcagaagaggacgagggccggggcgagaggaccgcctttatcaaagaccagagcgccctctga

SEQ ID NO: 109 - protein, 794 aa, Homo sapiens
MELRPWLLWVVAATGTLVLLAADAQGQKVFTNTWAVRIPGGPAVANSVARKHGFLNLGQIFGDYYHFWHRGVTKRSLSPHRPRHSRLQREPQVQWLEQQVAKRRTKRDVYQEPTDPKFPQQWYLSGVTQRDLNVKAAWAQGYTGHGIVVSILDDGIEKNHPDLAGNYDPGASFDVNDQDPDPQPRYTQMNDNRHGTRCAGEVAAVANNGVCGVGVAYNARIGGVRMLDGEVTDAVEARSLGLNPNHIHIYSASWGPEDDGKTVDGPARLAEEAFFRGVSQGRGGLGSIFVWASGNGGREHDSCNCDGYTNSIYTLSISSATQFGNVPWYSEACSSTLATTYSSGNQNEKQIVTTDLRQKCTESHTGTSASAPLAAGIIALTLEANKNLTWRDMQHLVVQTSKPAHLNANDWATNGVGRKVSHSYGYGLLDAGAMVALAQNWTTVAPQRKCIIDILTEPKDIGKRLEVRKTVTACLGEPNHITRLEHAQARLTLSYNRRGDLAIHLVSPMGTRSTLLAARPHDYSADGFNDWAFMTTHSWDEDPSGEWVLEIENTSEANNYGTLTKFTLVLYGTAPEGLPVPPESSGCKTLTSSQACVVCEEGFSLHQKSCVQHCPPGFAPQVLDTHYSTENDVETIRASVCAPCHASCATCQGPALTDCLSCPSHASLDPVEQTCSRQSQSSRESPPQQQPPRLPPEVEAGQRLRAGLLPSHLPEVVAGLSCAFIVLVFVTVFLVLQLRSGFSFRGVKVYTMDRGLISYKGLPPEAWQEECPSDSEEDEGRGERTAFIKDQSAL

SEQ ID NO: 110 - DNA, 19 bp, Artificial Sequence
  Description of Artificial Sequence Synthetic primer
aatggaccagttctaatgt

SEQ ID NO: 111 - DNA, 18 bp, Artificial Sequence
  Description of Artificial Sequence Synthetic primer
gtcagccctaaattcttc

SEQ ID NO: 112 - DNA, 20 bp, Artificial Sequence
  Description of Artificial Sequence Synthetic primer
taatacgactcactataggg

SEQ ID NO: 113 - DNA, 18 bp, Artificial Sequence
  Description of Artificial Sequence Synthetic primer
tagaaggcacagtcgagg

SEQ ID NO: 114 - DNA, 21 bp, Artificial Sequence
  Description of Artificial Sequence Synthetic primer
atggtgagcaagggcgaggag

SEQ ID NO: 115 - DNA, 21 bp, Artificial Sequence
  Description of Artificial Sequence Synthetic primer
cttgtacagctcgtccatgcc

SEQ ID NO: 116 - DNA, 29 bp, Artificial Sequence
  Description of Artificial Sequence Synthetic primer
ccggatcctgggaagcttgtcatcaacgg

SEQ ID NO: 117 - DNA, 27 bp, Artificial Sequence
  Description of Artificial Sequence Synthetic primer
ggctcgaggcagtgatggcatggactg

SEQ ID NO: 118 - protein, 8 aa, Unknown
  Description of Unknown ′SIINFEKL′ family peptide
SIINFEKL

SEQ ID NO: 119 - protein, 17 aa, Human herpesvirus 4
YLQQNWWTLLVDLLWLL

SEQ ID NO: 120 - protein, 16 aa, Artificial Sequence
  Description of Artificial Sequence Synthetic peptide
IGHVYIFATCLGLSYD

SEQ ID NO: 121 - protein, 8 aa, Mycoplasma penetrans
IYIFAACL

SEQ ID NO: 122 - protein, 15 aa, Homo sapiens
HQQYFYKIPILVINK

SEQ ID NO: 123 - protein, 15 aa, Homo sapiens
LLNWAYQQVQQNKED

SEQ ID NO: 124 - protein, 14 aa, Homo sapiens
EFHACWPAFTVLGE

SEQ ID NO: 125 - protein, 15 aa, Unknown
  Description of Unknown Survivin epitope peptide
WQPFLKDHRISTFKN

SEQ ID NO: 126 - protein, 15 aa, Human papillomavirus
LFVVYRDSIPHAACH

SEQ ID NO: 127 - protein, 15 aa, Human papillomavirus
GLYNLLIRCLRCQKP

SEQ ID NO: 128 - protein, 13 aa, Unknown
  Description of Unknown Carcinoembryonic antigen epitope peptide
LWWVNNQSLPVSP

SEQ ID NO: 129 - protein, 25 aa, Mycobacterium tuberculosis
FSKLPASTIDELKTNSSLLTSILTY

SEQ ID NO: 130 - protein, 28 aa, Mycobacterium tuberculosis
GNADVVCGGVSTANATVYMIDSVLMPPA

SEQ ID NO: 131 - protein, 13 aa, Unknown
  Description of Unknown HER-2 epitope peptide
GSPYVSRLLGICL

SEQ ID NO: 132 - protein, 17 aa, Unknown
  Description of Unknown HER-2 epitope peptide
KVPIKWMALESILRRRF

SEQ ID NO: 133 - protein, 25 aa, Unknown
  Description of Unknown NY-ESO-1 epitope peptide
PGVLLKEFTVSGNILTIRLTAADHR

SEQ ID NO: 134 - protein, 15 aa, Clostridium tetani
VSIDKFRIFCKANPK

SEQ ID NO: 135 - protein, 16 aa, Clostridium tetani
LKFIIKRYTPNNEIDS

SEQ ID NO: 136 - protein, 15 aa, Clostridium tetani
IREDNNITLKLDRCN

SEQ ID NO: 137 - protein, 21 aa, Clostridium tetani
FNNFTVSFWLRVPKVSASHLE

SEQ ID NO: 138 - protein, 14 aa, Clostridium tetani
QYIKANSKFIGITE

SEQ ID NO: 139 - protein, 20 aa, Hepatitis B virus
PHHTALRQAILCWGELMTLA

SEQ ID NO: 140 - protein, 13 aa, Influenza A virus
PKYVKQNTLKLAT

SEQ ID NO: 141 - protein, 15 aa, Hepatitis B virus
FFLLTRILTIPQSLD

SEQ ID NO: 142 - protein, 16 aa, Influenza A virus
YSGPLKAEIAQRLEDV

SEQ ID NO: 143 - protein, 18 aa, Plasmodium falciparum
EKKIAKMEKASSVFNVVN

(19) United States
(12) Patent Application Publication
(10) Pub. No.: US 2012/0225090 A1
(43) Pub. Date: Sep. 6, 2012

(54) METHODS FOR ENHANCING ANTIGEN-SPECIFIC IMMUNE RESPONSES

(75) Inventors: Tzyy-Choou Wu, Stevenson, MD (US); Chien-Fu Hung, Timonium, MD (US); Richard Roden, Severna Park, MD (US)

(73) Assignee: THE JOHNS HOPKINS UNIVERSITY, Baltimore, MD (US)

(21) Appl. No.: 13/388,889

(22) PCT Filed: Jul. 28, 2010
(86) PCT No.: PCT/US10/43544

§ 371 Date: May 15, 2012

Related U.S. Application Data
(60) Provisional application No. 61/230,848, filed on Aug. 3, 2009.

(51) Int. Cl. A61K 39/00; A61P 35/00; A61K 39/12; A61P 37/04

(52) U.S. Cl. 424/186.1; 424/204.1; 424/277.1; 424/185.1

(57) ABSTRACT

Methods for delivering naked DNA vaccines to enhance immune responses, by improving transfection efficiency without safety concerns associated with live viral vectors, are described. A method may comprise administering to a mammalian subject an effective amount of a papillomavirus pseudovirion, wherein the papillomavirus pseudovirion comprises at least one papillomavirus capsid protein encapsidating a naked DNA vaccine, wherein the naked DNA vaccine comprises a first nucleic acid encoding at least one antigen, thereby enhancing the antigen specific immune response relative to administration of the naked DNA vaccine.

### CROSS-REFERENCE TO RELATED APPLICATIONS

This application claims the benefit of U.S. Provisional Application No. 61/230,848, filed on Aug. 3, 2009, the entire contents of which are specifically incorporated by reference herein in its entirety.

### GOVERNMENTAL SUPPORT

This invention was made with government support under grant numbers 1 RO1 CA114425-01 and P50 CA 098252, awarded by the U.S. National Cancer Institute. The government has certain rights in this invention.

### SEQUENCE LISTING

The instant application contains a Sequence Listing which has been submitted in ASCII format via EFS-Web and is hereby incorporated by reference in its entirety. Said ASCII copy, created on Feb. 14, 2012, is named P1079703.txt and is 419,867 bytes in size.

### BACKGROUND

Cervical cancer is the second most common cause of cancer deaths in women worldwide. The primary factor in the development of cervical cancer is infection by human papilloma virus (HPV). HPV is one of the most common sexually transmitted diseases in the world. It is now known that cervical cancer is a consequence of persistent infection with high-risk type HPV. While most HPV-induced lesions are benign, lesions arising from certain papillomavirus types, e.g., HPV-16 and HPV-18, can undergo malignant progression. HPV infection is a necessary factor for the development and maintenance of cervical cancer and thus, effective vaccination against HPV to prevent infection by generating neutralizing antibodies represents an opportunity to prevent cervical cancer. While live viral vectors are capable of inducing potent cytotoxic T-cell immune responses, they raise significant concerns related to safety (e.g., malignancy). By contrast, current subunit vaccines and killed vaccines are safe and effective in inducing neutralizing antibodies and in preventing many new infections, but they have generally not proven effective in generating T-cell responses capable of clearing chronic viral infections (Roden et al., Expert Rev. Vaccines, 2:495-516 (2003)). Accordingly, naked nucleic acid (e.g., DNA) vaccines have been pursued in genetic vaccination strategies since they are stable, simple, inexpensive to manufacture, and safe. However, naked nucleic acid vaccines generally display lower immunogenicity in patients (Trimble et al., Clin. Cancer Res, 15:361-367 (2009) and Donnelly et al., J. Immunol., 175:633-639 (2005)). Thus, it is important to develop efficient mechanisms to deliver nucleic acid (e.g., DNA) vaccines in vivo without safety concerns and to increase antigen-specific immune responses.

### SUMMARY OF THE INVENTION

The present invention is based, at least in part, on methods of enhancing an antigen-specific immune response in a mammal, comprising administering to the subject an effective amount of a papillomavirus pseudovirion, wherein the papillomavirus pseudovirion comprises at least one papillomavirus capsid protein encapsidating a naked DNA vaccine, wherein the naked DNA vaccine comprises a first nucleic acid encoding at least one antigen, thereby enhancing the antigen specific immune response relative to administration of the naked DNA vaccine.

In one aspect, the papillomavirus pseudovirion comprises at least one furin-cleaved papillomavirus capsid protein.

In another aspect, the at least one papillomavirus capsid protein is a papillomavirus L1 protein and a papillomavirus L2 protein. In one embodiment, the papillomavirus L1 and L2 proteins are derived from HPV-2, HPV-16 or HPV-18. In another embodiment, the papillomavirus L1 protein comprises an amino acid sequence selected from the group consisting of SEQ ID NOs:97, 99, and 101, and the papillomavirus L2 protein comprises an amino acid sequence selected from the group consisting of SEQ ID NOs:103, 105 and 107.

In still another aspect, the antigen is a tumor-associated antigen (TAA).

In yet another aspect, the antigen is foreign to the mammal.

In another aspect, the antigen is selected from the group consisting of ovalbumin, HPV E6, and HPV E7. In one embodiment, the antigen comprises an ovalbumin protein comprising an amino acid sequence at least 90% identical to the amino acid sequence of SEQ ID NO:9. In another embodiment, the antigen comprises an HPV E6 protein comprising an amino acid sequence at least 90% identical to the amino acid sequence of SEQ ID NO:5 or a non-oncogenic mutant thereof. In still another embodiment, the antigen comprises an HPV E7 protein comprising an amino acid sequence at least 90% identical to the amino acid sequence of SEQ ID NO:2 or a non-oncogenic mutant thereof.

In still another aspect, the DNA vaccine further comprises a second nucleic acid encoding a fusion protein comprising an Ii protein, wherein the class II-associated Ii peptide (CLIP) region is replaced with the Pan HLA-DR reactive epitope (PADRE).

In yet another aspect, the DNA vaccine further comprises a second nucleic acid encoding a fusion protein comprising an Ii protein, wherein the class II-associated Ii peptide (CLIP) region is replaced with the Pan HLA-DR reactive epitope (PADRE).

In another aspect, the DNA vaccine further comprises a second nucleic acid that is (i) a siNA or (ii) DNA that encodes said siNA, wherein said siNA has a sequence that is sufficiently complementary to target the sequence of mRNA that encodes a pro-apoptotic protein expressed in a dendritic cell (DC) and results in inhibition of or loss of expression of said mRNA, thereby inhibiting apoptosis and increasing survival of DCs. In one embodiment, the pro-apoptotic protein is selected from the group consisting of one or more of (a) Bak, (b) Bax, (c) caspase-8, (d) caspase-9 and (e) caspase-3.

In still another aspect, the DNA vaccine further comprises a second nucleic acid encoding an anti-apoptotic polypeptide. In one embodiment, the anti-apoptotic polypeptide is selected from the group consisting of (a) BCL-xL (b) BCL2, (c) XIAP, (d) FLICEc-s, (e) dominant-negative easpase-8, (f) dominant negative caspase-9, (g) SPI-6 and (h) functional homologue or derivative of any of (a)-(g).

In yet another aspect, the DNA vaccine further comprises a second nucleic acid encoding an immunogenicity potentiating peptide (IPP), wherein the IPP acts in potentiating an immune response by promoting: (a) processing of the linked antigenic polypeptide via the MHC class I pathway or targeting of a cellular compartment that increases said processing; (b) development, accumulation or activity of antigen presenting cells or targeting of antigen to compartments of said antigen presenting cells leading to enhanced antigen presentation; c) intercellular transport and spreading of the antigen; or (d) any combination of (a)-(c). In one embodiment, the IPP is: (a) the sorting signal of the lysosome-associated membrane protein type 1 (Sig/LAMP-1); (b) mycoobacterial HSP70 polypeptide, the C-terminal domain thereof, or a functional homologue or derivative of said polypeptide or domain; (c) a viral intercellular spreading protein selected from the group of herpes simplex virus-1 VP22 protein, Marek's disease virus UL49 protein or a functional homologue or derivative thereof; (d) an endoplasmic reticulum chaperone polypeptide selected from the group of calreticulin or a domain thereof, ER60, GRP94, gp96, or a functional homologue or derivative thereof (e) domain II of Pseudomonas exotoxin ETA or a functional homologue or derivative thereof; (f) a polypeptide that targets the centrosome compartment of a cell selected from γ-tubulin or a functional homologue or derivative thereof; or (g) a polypeptide that stimulates DC precursors or activates DC activity selected from the group consisting of GM-CSF, Flt3-ligand extracellular domain, or a functional homologue or derivative thereof.

In one embodiment of any aspect of the present invention, the first and second nucleic acid sequences are comprised within at least one expression vector and are operatively linked to (a) a promoter; and (b) optionally, additional regulatory sequences that regulate expression of said nucleic acids in a eukaryotic cell. In another such embodiment, the first and second nucleic acid are operably linked either directly or via a linker.

In another aspect, the nucleic acid composition is papillomavirus pseudovirion is administered intradermally, intraperitoneally, or intravenously.

In still another aspect, the papillomavirus pseudovirion is administered to the subject by: (a) priming the mammal by administering to the mammal an effective amount of the papillomavirus pseudovirion; and (b) boosting the mammal by administering to the mammal an effective amount of the papillomavirus pseudovirion, thereby inducing or enhancing the antigen-specific immune response. In one embodiment, the papillomavirus pseudovirions administered in steps (a) and (b) comprise the same type of capsid protein composition to thereby produce homologous vaccination. In another embodiment, the papillomavirus pseudovirions administered in steps (a) and (b) comprise different types of capsid protein compositions to thereby produce heterologous vaccination. In still another embodiment, the step (a) and/or step (b) is repeated at least once.

In yet another aspect, the antigen-specific immune response is mediated at least in part by CD8⁺ cytotoxic T lymphocytes (CTL).

In another aspect, the pseudovirions infect bone marrow-derived dendritic cells (BMDCs). In one embodiment, the BMDCs are selected from the group consisting of B220+ cells and CD11c+ cells.

In still another aspect, the methods of the present invention further comprise administering an effective amount of a chemotherapeutic agent.

In yet another aspect, the methods of the present invention further comprise screening the mammal for the presence of antibodies against the antigen.

In another aspect, the methods of the present invention are applied to a mammal wherein the mammal is a human and/or wherein the mammal is afflicted with cancer.

### BRIEF DESCRIPTION OF THE DRAWINGS

FIGS. 1A-1B. OVA-specific CD8+ T cell immune responses generated by HPV-16 pseudovirion vaccination. Representative flow cytometry data demonstrating the number of OVA-specific CD8+ T cells generated by vaccination with HPV16-OVA or HPV16-pcDNA3 pseudovirions are shown. 5-8 week old C57BL/6 mice (5 per group) were vaccinated with HPV16-OVA or HPV16-pcDNA3 pseudovirions (5 μg L1 protein/mouse) via footpad injection. All mice were boosted 7 days later with the same regimen. 1 week after last vaccination, splenocytes were prepared and stimulated with OVA peptide, SIINFEKL (SEQ ID NO: 118) (1 μg/ml) in the presence of GolgiPlug overnight at 37° C. The OVA-specific CD8⁺ T cells were then analyzed by intracellular cytokine staining followed by flow cytometry analysis. (A) Representative flow cytometry data are shown demonstrating the number of OVA-specific CD8+ T cells generated by vaccination with HPV-16-OVA pseudovirions. (B) A graphical representation of the number of OVA-specific CD8+ T cells/3×10⁵ splenocytes is shown.

FIG. 2. Characterization of the OVA-specific CD4+ T cell responses generated by subcutaneous HPV16-OVA pseudoviruses vaccination. 5-8 week old C57BL/6 mice were vaccinated with 5 μg of HPV 16-OVA pseudovirus (L1 protein amount) via footpad injection. All mice were boosted 7 days later with the same regimen. 1 week after last vaccination, splenocytes were prepared and stimulated with OVA MHC class II peptide (OVAaa323-339) at 2 μg/ml at the presence of GolgiPlug overnight at 370 C. The OVA-specific CD4+ T cells were then analyzed by staining surface CD4 and intracellular IFN-γ.

FIG. 3. Characterization of the OVA-specific antibody responses generated by subcutaneous HPV16-OVA pseudoviruses vaccination. 5-8 week old C57BL/6 mice were vaccinated with 5 μg of HPV 16-OVA pseudovirus (L1 protein amount) via footpad injection. All mice were boosted 7 days later with the same regimen. OVA protein based ELISA was performed to detect OVA-specific antibody response, either 1, 2 or 3 weeks after the initial vaccination. OVA protein was used as a positive control.

FIG. 4. Induction of HPV 16-specific neutralization antibody responses by subcutaneous HPV 16-OVA pseudoviruses vaccination. 5-8 week old C57BL/6 mice were vaccinated with 5 μg of HPV 16-OVA pseudovirus (L1 protein amount) via footpad injection. All mice were boosted 7 days later with the same regimen. Sera were collected from those mice at d0, d7, d14 and d21. In vitro neutralization assays were performed using HPV 16-SEAP pseudovirus on two-fold dilutions of the sera collected from the vaccinated mice 2 weeks. Endpoint titers achieving 50% neutralization are plotted and the means shown as horizontal lines.

FIGS. 5A-5B. Comparison of OVA-specific CD8⁺ T cell responses induced by homologous or heterologous pseudovirion boost. Representative flow cytometry data are shown demonstrating the number of OVA-specific CD8+ T cells generated by homologous or heterologous vaccination with HPV-OVA pseudovirions. 5-8 week old C57BL/6 mice (5 per group) were vaccinated with indicated HPV16-OVA pseudovirions (5 μg L1 protein/mouse) via either intramuscular, or subcutaneous (footpad) injection. 7 days later, one group was boosted with HPV16-OVA pseudovirions, and another group was boosted with HPV18-OVA pseudovirions. 1 week after last vaccination, splenocytes were prepared and stimulated with OVA peptide, SIINFEKL (SEQ ID NO: 118) (1 μg/ml) in the presence of GolgiPlug overnight at 37° C. The OVA-specific CD8⁺ T cells were then analyzed by staining surface CD8 and intracellular IFN-γ. (A) Representative flow cytometry data are shown demonstrating the number of OVA-specific CD8+ T cells generated by homologous or heterologous vaccination with pseudovirions. (B) A graphical representation of the number of OVA-specific CD8+ T cells/3×10⁵ splenocytes is shown.

FIGS. 6A-6B. Dose responses of OVA-specific CD8⁺ T cell responses induced by HPV16-OVA pseudovirion vaccination. 5-8 week old C57BL/6 mice (5 per group) were vaccinated with different doses of HPV 16-OVA pseudovirions (0.1-5 μg L1 protein/mouse) via subcutaneous (footpad) injection. 7 days later, the mice were boosted with the same amount of HPV16-OVA pseudovirions via footpad injection. 1 week after last vaccination, splenocytes were prepared and stimulated with OVA peptide, SIINFEKL (SEQ ID NO: 118) (1 μg/ml) in the presence of GolgiPlug overnight at 37° C. The OVA-specific CD8⁺ T cells were then analyzed by intracellular cytokine staining followed by flow cytometry analysis. (A) Representative flow cytometry data are shown demonstrating the number of OVA-specific CD8+ T cells generated by vaccination with different doses of HPV16-OVA pseudovirions. (B) A graphical representation of the number of OVA-specific CD8+ T cells/3×10⁵ splenocytes is shown.

FIGS. 7A-7C. Characterization of OVA-specific CD8+ T cell immune responses generated by HPV-16 L1 mutant L2-OVA pseudovirion vaccination. (A) Representative flow cytometry data are shown demonstrating the activation of OVA-specific CD8+ T cells generated by HPV16 L2 mutated or wild-type HPV16-OVA pseudovirus infected 293-Kb cells. 293-Kb cells were infected with HPV16L1L2-OVA or HPV16L1mtL2-OVA pseudovirus (4 μg of L1 protein) for 72 hours. These cells were co-incubated with OT-I T cells at the E:T ratio of 2:1 at the presence of GolgiPlug overnight. OT-I T cell activation was then analyzed with intracellular IFN-γ staining. (B and C) 5-8 week old C57BL/6 mice (5 per group) were vaccinated with HPV16L1L2-OVA or HPV16L1mtL2-OVA pseudoviruses (5 μg of L1 protein/mouse) via footpad injection. All mice were boosted 7 days later with the same regimen. 1 week after last vaccination, splenocytes were prepared and stimulated with OVA peptide, SIINFEKL (SEQ ID NO: 118) (1 μg/ml) in the presence of GolgiPlug overnight at 37° C. The OVA-specific CD8⁺ T cells were then analyzed by staining surface CD8 and intracellular IFN-γ by intracellular cytokine staining followed by flow cytometry analysis. (B) Representative flow cytometry data are shown demonstrating the number of OVA-specific CD8+ T cells generated by vaccination with the different pseudovirions. (C) A graphical representation of the number of OVA-specific CD8+ T cells/3×10⁵ splenocytes is shown.

FIGS. 8A-8B. In vivo tumor protection experiments. 5-8 week old C57BL/6 mice were vaccinated with HPV16-OVA (5 μg of L1 protein/mouse) or HPV16-pcDNA3 via footpad injection. The mice were boosted twice with the same regimen at day 7 and day 14. One week after last vaccination, the mice were injected with 1×10⁵ B16-OVA cells subcutaneously. (A) Kaplan Meier survival analysis of the groups of mice vaccinated with HPV16-pcDNA3 or HPV16-pcDNA3-OVA is shown. (B) Kaplan Meier survival analysis of the groups of mice vaccinated with HPV16-pcDNA3 or HPV16-pcDNA3-OVA and depleted of CD4, CD8 or NK cells is shown. For the antibody depletion experiment, mice were treated with antibodies against mouse CD4, CD8 or NK1.1 at the same time of last vaccination via intraperitoneal injection. One week after last vaccination, the mice were injected with 1×10⁵ B16-OVA cells subcutaneously. Tumor growth was monitored twice a week. Representative data from one of three independent experiments are shown.

FIGS. 9A-9B. Comparison of OVA-specific CD8⁺ T cell responses induced by pseudovirion or DNA vaccination. 5-8 week old C57BL/6 mice (5 per group) were vaccinated with HPV16-OVA pseudovirions (5 μg L1 protein/mouse) via subcutaneous (footpad) injection, or vaccinated with 2 μg of pcDNA3-OVA via gene gun delivery. These mice were boosted 7 days later with the same regimen. 1 week after last vaccination, splenocytes were prepared and stimulated with OVA peptide, SIINFEKL (SEQ ID NO: 118) (1 μg/ml) in the presence of GolgiPlug overnight at 37° C. The OVA-specific CD8⁺ T cells were then analyzed by intracellular cytokine staining followed by flow cytometry analysis. (A) Representative flow cytometry data are shown demonstrating the number of OVA-specific CD8+ T cells generated by vaccination with HPV-16-OVA pseudovirions or OVA DNA. (B) A graphical representation of the number of OVA-specific CD8+ T cells/3×10⁵ splenocytes is shown.

FIGS. 10A-10D. Analysis of cells infected by HPV pseudovirion. (A) In vitro infection of BMDCs by HPV pseudovirus. BMDCs were generated from bone marrow progenitor cells and infected with HPV16-GFP or HPV16-OVA pseudovirus at day 4 (4 μg L1 protein). After 72 hours, BMDCs were harvested and GFP expression was examined by flow cytometry. (B) RT-PCR to demonstrate the expression of GFP mRNA in draining lymph nodes of mice infected with HPV16 pseudovirions containing GFP or OVA. 5-8 week old C57BL/6 mice were vaccinated with 10 μg/mouse of HPV16 pseudovirions carrying GFP or OVA DNA subcutaneously. After 72 hours, draining lymph nodes were harvested and total RNA was isolated with TRIzol. RT-PCR was then performed to detect GFP mRNA expression. (C) Representative flow cytometry data depicting the percentage of CD11c+ cells and B220+ cells that uptake the FITC-labeled pseudovirions are shown. HPV16-OVA pseudovirus was labeled with FITC. 5-8 week old C57BL/6 mice were given 10 μg/mouse of HPV16-OVA or HPV16-OVA-FITC pseudovirus subcutaneously. After 72 hours, draining lymph nodes were harvested, and digested with 0.05 mg/ml Collagenase I, 0.05 mg/ml collagenase IV, 0.025 mg/ml Hyaluronidase IV and 0.25 mg/ml DNase I. The cells were then stained with anti-mouse CD11c-APC and PE-Cy5-conjugated anti-mouse B220 followed by flow cytometry analysis. (D) A representative bar graph depicting the percentage of FITC+ CD11c+ cells and FITC+ B220+ cells is shown.

FIGS. 11A-11C. Characterization of the infection and antigen presentation of HPV16-GFP pseudovirions treated with furin. (A) Representative flow cytometry data are shown demonstrating the percentage of GFP expressing DC-1 cells. A dendritic cell line, DC-1, was infected with 4 μg (L1 protein) of HPV16-GFP or HPV16-OVA pseudovirions with or without the presence of Furin (5 units). After 72 hours, GFP expression by DC-1 cells was analyzed by flow cytometry. (B) Representative flow cytometry data are shown demonstrating the percentage of activated OVA-specific CD8+ T cells. Infected DC-1 cells were collected 72 hours after infection, and co-cultured with OVA-specific OT-1 T cells (E:T ratio at 1:1) at the presence of GolgiPlug overnight. Activation of OT-1 T cells was analyzed by IFN-γ intracellular staining (C) Results of intracellular cytokine staining followed by flow cytometry analysis to characterize the number of OVA-specific CD8+ T cells in mice vaccinated with HPV 16-OVA pseudovirions with or without furin treatment are shown. FIG. 11(C) discloses “SIINFEKL” as SEQ ID NO: 118.

FIG. 12. Characterization of infection of mouse skin using HPV-2 pseudovirions carrying luciferase gene. A patch of skin on the ventral torso of anesthetized BALB/c mice was prepared for infection by shaving the abdominal region. Infection of mouse skin was performed by application of 3×10⁹ luciferase-expressing HPV-2 pseudovirion particles (5 μg L1 protein/mouse) in 20 μl of 3% carboxymethylcellulose (CMC; Sigma-Aldrich) to the epithelial patches. Mice transfected with equivalent amount of naked luciferase DNA (50 ng) or PBS were used as controls. 3 days later, mice were reanesthetized, injected with luciferin (800 μl at 3 mg/ml), and imaged for 10 min with IVIS 200 bioluminescent imaging system (Xenogen) using methods. Equal areas encompassing the site of virus inoculation were analyzed by using Living Image 2.20 software.

FIG. 13. Characterization of infection of human skin using HPV-2 pseudovirions carrying luciferase gene. Patches (10×20×0.5 mm) of human breast skin from surgical discards were obtained through Johns Hopkins Department of Pathology and placed in a 6 well plate. Skin patches were submerged, but not covered, by RPMI 1640 culture medium. Infection of human skin was performed by application of 3×10⁹ luciferase-expressing HPV-2 pseudovirion particles (5 μg L1 protein) in 20 μl of medium to the epithelial patches. Human skin transfected with equivalent amount of naked luciferase DNA (50 ng/20 ul) or with PBS were used as controls. 1 hr later, culture medium was brought up to volume of 1 cc. 3 days later, luminescence imaging was performed by adding luciferin (200 μl at 3 mg/ml), and imaged for 5 min with IVIS 200 bioluminescent imaging system (Xenogen).

### DETAILED DESCRIPTION

The inventors of the present invention have determined that papillomavirus pseudovirions represents a novel approach for the delivery of naked DNA vaccines to improve transfection efficiency without safety concerns associated with live viral vectors. Accordingly, the present invention is drawn to methods for enhancing an antigen-specific immune response in a mammal using recombinant papillomavirus pseudovirions comprising an antigen.

### Partial List of Abbreviations

ANOVA, analysis of variance; APC, antigen presenting cell; CRT, calreticulin; CTL, cytotoxic T lymphocyte; DC, dendritic cell; E6, HPV oncoprotein E6; E7, HPV oncoprotein E7; ELISA, enzyme-linked immunosorbent assay; HPV, human papillomavirus; IFN γ, interferon-γ; i.m., intramuscular(ly); i.t., intratumoral(ly); i.v., intravenous(ly); luc, luciferase; mAB, monoclonal antibody; MOI, multiplicity of infection; OVA, ovalbumin; p-, plasmid-; PBS, phosphate-buffered saline; PCR, polymerase chain reaction; SD, standard deviation; TAA, tumor-associate antigen; WT, wild-type.

### Pseudovirions

Papillomaviruses are non-enveloped double-stranded DNA viruses about 55 nm in diameter harboring an approximately 8 kb genome in their nucleohistone core (Baker et al., Biophys. J. 60:1445 (1991)). The capsids are composed of two virally-encoded proteins, L1 and L2, that migrate on SDS-PAGE gels at approximately 55 kDa and 75 kDa, respectively (Larson et al., J. Virol. 61:3596 (1987)). L1, which is the major capsid protein, is arranged in 72 pentameters which associate with T=7 icosahedral symmetry. The L1 protein has the capacity to self-assemble so that large amounts of virus-like particles (VLPs) may be generated by expression of the L1 protein from a number of species of papillomavirus in a variety of recombinant expression systems (Hagensee et al., J. Virol. 67:315 (1993); Kirnbauer et al., Proc. Natl. Acad. Sci. USA 89:12180 (1992); Kirnbauer et al., J. Virol. 67:6929 (1993); Rose et al., J. Virol. 67:1936 (1993)). Although not required for assembly, L2 is incorporated into VLPs when co-expressed with L1 (L1/L2 VLPs) in cells. Indeed, purified L1 protein can be used to generate papillomavirus vectors in the absence of L2 using cell-free production systems, including intracellular encapsidation of nucleic acids (Kawana et al., J. Virol. 72:10298-10300; Muller et al., J. Virol. 69:948-954; Touze and Coursaget, Nuc. Acids Res. 26:1317-1323; Unckell et al., J. Virol. 71:2934-2945; Yeager et al., Virol. 278:570-577).

The inventors of the present invention have determined that pseudovirions (i.e., non-replicative viral particles; also referred to as pseudo viruses) can be engineered to facilitate the delivery of naked nucleic acid (e.g., DNA) vaccines based upon encapsidation of such vaccines within papillomavirus capsid proteins. Such enhanced nucleic acid (e.g., DNA) vaccine delivery is quite different from known delivery systems using VLPs since VLPs carry no genetic information (i.e., no nucleic acids). Thus, delivery of DNA using VLPs require either the binding of DNA to VLPs or the in vitro assembly of DNA within the VLPs (Malboeuf et al., Vaccine, 25:3270-3276 (2007); E1 Mehdaoui et al., J. Virol., 74:10332-10340 (2000); Zhang et al., J. Virol., 78:10249-10257 (2004); Bousarghin et al., J. Clin. Microbiol., 40:926-932 (2002); Combita et al., FEMS Microbiol. Lett., 204:183-188 (2001); and U.S. Patent Publication No. 2006/0269954). Such processes do not appreciate the importance of the minor capsid protein L2 or need for infection by papillomavirus particles for gene delivery in order to generate antigen specific immune responses in vivo. By contrast, the pseudovirions used in the methods of the present invention employ packaging of nucleic acid vaccines by papillomavirus capsid proteins within cells used for papillomavirus pseudovirion production purposes, as well as the inclusion of L2 protein for efficient infection of target cells.

Accordingly, the methods of the present invention use papillomaviral pseudovirions. Such pseudovirions can comprise either L1 capsid protein alone, or both L1 and L2 capsid proteins together. Pseudovirions comprising both L1 and L2 (i.e., L1/L2) capsid proteins are more closely related to the composition of native papillomavirus virions, but it is believed in the art that L2 does not appear to be as significant as L1 in conferring immunity, probably because most of L2 is internal to L1 in the capsid structure. However, the inventors of the present invention have unexpectedly determined that the L2 minor capsid protein is important for the generation of antigen-specific CD8+ T-cell responses in vaccinated animal models because it is important for in vivo pseudovirion infectivity, as opposed to anti-papillomavirus vaccination purposes focused upon in the field.

The methods of the present invention are not particularly limited by the use of capsid protein(s) from specific papillomaviruses. For example, many human subjects in need of enhancing antigen-specific immune responses may have previously been infected or vaccinated with human papillomaviruses (e.g., HPV-2, HPV-16 or HPV-18), which could preclude repeated vaccination with pseudovirions comprising capsid proteins from the same papillomaviral type. Accordingly, many other types of HPVs and papillomaviruses from different species can be used for the preparation of pseudovirions for the delivery of nucleic acid (e.g., DNA) vaccines according to the methods of the present invention. In some embodiments, the source of the capsid protein encoding genes may be any papillomavirus, human or non-human. In other embodiments, the source of such genes can include human papillomavirus serotypes, including one or more of HPV-1, HPV-2, HPV-6a, HPV-6b, HPV-11, HPV-13, HPV-16, HPV-18, HPV-30, HPV-31, HPV-33, HPV-35, HPV-39, HPV-40, HPV-41, HPV-42, HPV-44, HPV-45, HPV-47, HPV-51, HPV-52, HPV-53, HPV-56, HPV-57, HPV-58, HPV-59, HPV-61, HPV-64, and/or HPV-68. In still other embodiments, the source of such genes can include animal papillomaviruses, especially those from papillomaviruses used in animal disease models, such as monkey (e.g., macaca fascicularis MfPV or macaca mulatta MmPV), cottontail rabbit papillomavirus (CRPV), bovine papillomavirus (BPV such as BPV1) and canine oral papillomavirus (COPV). The sequences of numerous human and animal papillomavirus capsid encoding genes are well known in the art. In one embodiment, pseudovirions of the present invention comprise L1 and L2 capsid protein expressed by a wild type HPV genome (e.g., HPV-2, HPV-16 or HPV-18), either as L1 alone or L1/L2 together.

In another aspect of the present invention, the pseudovirions can comprise papillomaviral capsid protein(s) engineered for yielding high-titers in expression systems useful to generate large quantities of pseudovirions for vaccination. It is well known in the art that papillomavirus L1 and L2 capsid genes are generally expressed at low levels in in vitro expression systems. Accordingly, codons encoding amino acids for which corresponding tRNAs are rare in the specific expression system can be replaced with codons using more common tRNAs. Alternatively, cis-acting elements that inhibit RNA production, processing, and translation can be engineered to disinhibit such processes. The sequences of numerous such engineered human and animal papillomavirus capsid encoding genes are well known in the art (Buck et al., J. Virol. 78, 751-757 (2004); Bambhira et al. Virol. J. 6:176 (2009); U.S. Pat. Nos. 6,599,739, 7,205,126, and 6,416,945; and Buck and Thomspon, Curr. Prot. Cell Biol. 26.1.1-26.1.19 (2007); herein incorporated in their entirety by this reference). Chimeric proteins containing conservative amino acid substitutions that do not affect the conformation of correctly folded proteins are further included. Such substitutions can be generated in the course of constructing the chimeric molecules, such as through site-specific mutagenesis, conserved restriction endonuclease sites, and the like. In one embodiment, pseudovirions of the present invention comprise L1 and L2 capsid protein expressed by a wild type HPV genome (e.g., HPV-2, HPV-16 or HPV-18), either as L1 alone or L1/L2 together, but have been further engineered to increase titer in expression systems. Representative L1 nucleic acid and polypeptide sequences are provided herein as SEQ ID NOs: 96 (HPV-16) and 97 (HPV-16); SEQ ID NOs: 98 (HPV-18) and 99; and 100 (HPV-2) and 101 (HPV-2), respectively. L1 nucleic acid and polypeptide sequences from other papillomaviruses are well known in the art and include, for example, MfPV-9 (YP_{—}002860301.1); MmPV-1 (NP_{—}043338.1); MfPV-10 (YP_{—}002860309.1); MfPV-7 (YP_{—}002854757.1); HPV-34 (NP_{—}041812.1); HPV-32 (NP_{—}041806.1); HPV-10 (NP_{—}041746.1 and NP_{—}041747.1); HPV-54 (NP_{—}043294.1); HPV-7 (NP_{—}041859.1); HPV-6b (NP_{—}040304.1); HPV-26 (NP_{—}041787.1); HPV-114 (YP_{—}003495077.1); HPV-53 (NP_{—}041848.1); HPV-61 (NP_{—}043450.1); HPV-71 (NP_{—}597938.1); Ursus maritimus PV-1 (YP_{—}001931973.1); Sus scrofa PV-1 (YP_{—}002235542.1); rattus norvegicus PV-1 (YP_{—}003169705.1); HPV-96 (NP_{—}932325.1); HPV-63 (NP_{—}040902.1); procyon lotor PV-1 (YP_{—}249604.1); HPV-9 (NP_{—}041866.1); HPV-1 (NP_{—}040309.1); rabbit oral PV (NP_{—}057848.1); HPV-104 (YP_{—}002922928.1); HPV-98 (YP_{—}002922755.1); HPV-49 (NP_{—}041837.1); HPV-113 (YP_{—}002922781.1); cottontail rabbit PV (NP_{—}077113.1); canine PV-5 (YP_{—}003204674.1); HPV-99 (YP_{—}002922761.1); HPV-109 (YP_{—}002756544.1); HPV-4 (NP_{—}040895.1); HPV-115 (YP_{—}003331603.1); HPV-24 (NP_{—}043373.1); HPV-92 (NP_{—}775311.1); HPV-5 (NP_{—}041372.1); HPV-112 (YP_{—}002756551.1); HPV-105 (YP_{—}002922774.1); HPV-60 (NP_{—}043443.1); HPV-103 (YP_{—}656498.1); BPV-9 (YP_{—}001648349.1); BPV-10 (YP_{—}001648356.1); HPV-108 (YP_{—}002647038.1); BPV-3 (NP_{—}694451.1); HPV-101 (YP_{—}656504.1); equine PV-2 (YP_{—}002635574.1); HPV-121 (YP_{—}003668031.1); HPV-48 (NP_{—}043422.1); HPV-88 (YP_{—}001672014.1); HPV-116 (YP_{—}003084352.1); and HPV-50 (NP_{—}043429.1). Nucleic acid sequences encoding such L1 polypeptides are well known in the art and can be made and used according to methods further described herein and knowledge readily available in the art.

Representative L2 nucleic acid and polypeptide sequences are provided herein as SEQ ID NOs: 102 (HPV-16) and 103 (HPV-16); 104 (HPV-18) and 105 (HPV-18); and 106 (HPV-2) and 107 (HPV-2), respectively. L2 nucleic acid and polypeptide sequences from other papillomaviruses are well known in the art and include, for example, MfPV-10 (YP_{—}002860308.1); MfPV-9 (YP_{—}002860300.1); MfPV-7 (YP_{—}002854756.1); HPV-6b (NP_{—}040303.1); HPV-114 (YP_{—}003495076.1); HPV-61 (NP_{—}043449.1); HPV-10 (NP_{—}041745.1); HPV18 (NP_{—}040316.1); HPV-71 (NP_{—}597937.1); ursus maritimus PV-1 (YP_{—}001931972.1); sus scrofa PV-1 (YP_{—}002235541.1); HPV-115 (YP_{—}003331602.1); rabbit oral PV (NP_{—}057847.1); HPV-104 (YP_{—}002922927.1); HPV-5 (NP_{—}041371.1); HPV-99 (YP_{—}002922760.1); HPV-98 (YP_{—}002922754.1); canine PV-4 (YP_{—}001648804.1); HPV-100 (YP_{—}002922767.1); HPV-113 (YP_{—}002922780.1); HPV-101 (YP_{—}656503.1); HPV-109 (YP_{—}002756543.1); HPV-1 (NP_{—}040308.1); HPV-105 (YP_{—}002922773.1); canine PV-6 (YP_{—}003204680.1); HPV-92 (NP_{—}775310.1); HPV-108 (YP_{—}002647037.1); HPV-50 (NP_{—}043428.1); HPV-96 (NP_{—}932324.1); cottontail rabbit PV (NP_{—}077112.1); bovine PV-3 (NP_{—}694450.1); HPV-121 (YP_{—}003668030.1); canine PV-5 (YP_{—}003204673.1); canine PV-2 (YP_{—}164634.1); HPV-103 (YP_{—}656497.1); bovine PV-9 (YP_{—}001648348.1); HPV-48 (NP_{—}043421.1); bovine PV-10 (YP_{—}001648355.1); HPV-60 (NP_{—}043442.1); HPV-88 (YP_{—}001672013.1); HPV-112 (YP_{—}002756550.1); equine PV-2 (YP_{—}002635573.1); bovine PV-8 (YP_{—}001429550.1); and HPV-116 (YP_{—}003084351.1). Nucleic acid sequences encoding such L1 polypeptides are well known in the art and can be made and used according to methods further described herein and knowledge readily available in the art.

In still another aspect of the present invention, the present inventors have unexpectedly determined that treatment of papillomavirus pseudovirions with furin leads to enhanced pseudovirion infection, both in vitro and in vivo, and that such treatment improves antigen presentation in infected cells. Accordingly, in one embodiment, the methods of the present invention can use papillomaviral capsid proteins described above that have been further treated with furin. Furin proteins are well known in the art as proteases that recognize and cleave polypeptides at specific amino acid recognition motifs (e.g., Arg-X-X-Arg). In another embodiment, the furin treatment occurs within the pseudovirion expression extract before the maturation process. The sequences of numerous furin encoding genes suitable for use in the present invention, as well as methods for treating papillomavirus capsid proteins with such furins, are well known in the art (Day et al., J. Virol. 82:12565-12568 (2008); herein incorporated in its entirety by this reference). Representative furing nucleic acid and polypeptide sequences are provided herein as SEQ ID NOs: 108 and 109, respectively. Furin nucleic acid and polypeptide sequences from species other than humans are well known in the art and include, for example, from canis familiaris (XM_{—}545864.2 and XP_{—}545864.2); pan troglodytes (XM_{—}510596.2 and XP_{—}510596.2); bos taurus (NM_{—}174136.2 and NP_{—}776561.1); rattus norvegicus (NM_{—}019331.1 and NP_{—}062204.1); and mus musculus (NM_{—}011046.2 and NP_{—}035176.1).

Production of the recombinant L1, or L1/L2 pseudovirions, as well as furin, can be carried out by cloning the L1 (or L1 and L2 or furin) gene(s) into a suitable vector and expressing the corresponding conformational coding sequences for these proteins in a eukaryotic cell transformed by the vector according to well known methods in the art (especially as those taught in the Examples and references cited therein). The gene(s) is preferably expressed in a eukaryotic cell system. In one embodiment, human cells, such as human embryonic kidney 293 cells, are used. However, insect and yeast-cell based expression systems are also suitable. Other mammalian cells similarly transfected using appropriate mammalian expression vectors can also be used to produce assembled pseudovirions. Suitable vectors for cloning of expression of the recited DNA sequences are well known in the art and commercially available. Further, suitable regulatory sequences for achieving cloning and expression, e.g., promoters, polyadenylation sequences, enhancers and selectable markers are also well known. The selection of appropriate sequences for obtaining recoverable protein yields is routine to one skilled in the art.

### Nucleic Acid (e.g., DNA) Vaccines

Vaccines that may be administered to a mammal include any vaccine, e.g., a nucleic acid vaccine (e.g., a DNA vaccine). In an embodiment of the invention, a nucleic acid vaccine will encode an antigen, e.g., an antigen against which an immune response is desired. Other nucleic acids that may be used are those that increase or enhance an immune reaction, but which do not encode an antigen against which an immune reaction is desired. These vaccines are further described below.

Exemplary antigens include proteins or fragments thereof from a pathogenic organism, e.g., a bacterium or virus or other microorganism, as well as proteins or fragments thereof from a cell, e.g., a cancer cell. In one embodiment, the antigen is from a virus, such as class human papillomavirus (HPV), e.g., E7 or E6. These proteins are also oncogenic proteins, which are important in the induction and maintenance of cellular transformation and co-expressed in most HPV-containing cervical cancers and their precursor lesions. Therefore, cancer vaccines that target E7 or E6 can be used to control of HPV-associated neoplasms (Wu, T-C, Curr Opin Immunol. 6:746-54, 1994).

However, as noted, the present invention is not limited to the exemplified antigen(s). Rather, one of skill in the art will appreciate that the same results are expected for any antigen (and epitopes thereof) for which a T cell-mediated response is desired. The response so generated will be effective in providing protective or therapeutic immunity, or both, directed to an organism or disease in which the epitope or antigenic determinant is involved—for example as a cell surface antigen of a pathogenic cell or an envelope or other antigen of a pathogenic virus, or a bacterial antigen, or an antigen expressed as or as part of a pathogenic molecule.

Exemplary antigens and their sequences are set forth below.

E7 Protein from HPV-16

The E7 nucleic acid sequence (SEQ ID NO:1) and amino acid sequence (SEQ ID NO:2) from HPV-16 are shown herein (see GenBank Accession No. NC_{—}001526). The single letter code, the wild type E7 amino acid sequence (SEQ ID NO:2) is shown herein.

In another embodiment (See GenBank Accession No. AF125673, nucleotides 562-858 and the E7 amino acid sequence), the C-terminal four amino acids QDKL (residues 96-99 of SEQ ID NO: 2) (and their codons) above are replaced with the three amino acids QKP (and the codons cag aaa cca), yielding a protein of 98 residues.

When an oncoprotein or an epitope thereof is the immunizing moiety, it is preferable to reduce the tumorigenic risk of the vaccine itself. Because of the potential oncogenicity of the HPV E7 protein, the E7 protein may be used in a “detoxified” form.

To reduce oncogenic potential of E7 in a construct of the present invention, one or more of the following positions of E7 is mutated:

| | | | | Amino |
|---|---|---|---|---|
| | | Preferred | nt Position | acid (in |
| Original | Mutant | codon | (in SEQ ID | SEQ ID |
| residue | residue | mutation | NO: 1) | NO: 2) |
| Cys | Gly (or Ala) | TGT→GGT | 70 | 24 |
| Glu | Gly (or Ala) | GAG→GGG | 77 | 26 |
| | | (or GCG) | | |
| Cys | Gly (or Ala) | TGC→GGC | 271 | 91 |

In one embodiment, the E7 (detox) mutant sequence has the following two mutations:

a TGT→GGT mutation resulting in a Cys→Gly substitution at position 24 of SEQ ID NO: 9 and GAG→GGG mutation resulting in a Glu→Gly substitution at position 26 of the wild type E7. This mutated amino acid sequence is shown herein as SEQ ID NO:3.

These substitutions completely eliminate the capacity of the E7 to bind to Rb, and thereby nullify its transforming activity. Any nucleotide sequence that encodes the above E7 or E7(detox) polypeptide, or an antigenic fragment or epitope thereof, can be used in the present compositions and methods, including the E7 and E7(detox) sequences which are shown herein.

E6 Protein from HPV-16

The wild type E6 nucleotide (SEQ ID NO:4) and amino acid sequences (SEQ ID NO:5) are shown herein (see GenBank accession Nos. K02718 and NC_{—}001526). This polypeptide has 158 amino acids and is shown herein in single letter code as SEQ ID NO:5.

E6 proteins from cervical cancer-associated HPV types such as HPV-16 induce proteolysis of the p53 tumor suppressor protein through interaction with E6-AP. Human mammary epithelial cells (MECs) immortalized by E6 display low levels of p53. HPV-16 E6, as well as other cancer-related papillomavirus E6 proteins, also binds the cellular protein E6BP (ERC-55). As with E7, described below a non-oncogenic mutated form of E6 may be used, referred to as “E6(detox).” Several different E6 mutations and publications describing them are discussed below.

The amino acid residues to be mutated are underscored in the E6 amino acid sequence provided herein. Some studies of E6 mutants are based upon a shorter E6 protein of 151 nucleic acids, wherein the N-terminal residue was considered to be the Met at position 8 in the wild type E6. That shorter version of E6 is shown herein as SEQ ID NO:6.

To reduce oncogenic potential of E6 in a construct, one or more of the following positions of E6 is mutated:

| Original | Mutant | aa position in | aa position in |
|---|---|---|---|
| residue | residue | SEQ ID NO: 5 | SEQ ID NO: 6 |
| Cys | Gly (or Ala) | 70 | 63 |
| Cys | Gly (or Ala) | 113 | 106 |
| Ile | Thr | 135 | 128 |

Nguyen et al., J. Virol. 6:13039-48, 2002, described a mutant of HPV-16 E6 deficient in binding α-helix partners which displays reduced oncogenic potential in vivo. This mutant, which includes a replacement of Ile with Thr as position 128 (of SEQ ID NO: 6), may be used in accordance with the present invention to make an E6 DNA vaccine that has a lower risk of being oncogenic. This E6(I¹²⁸T) mutant is defective in its ability to bind at least a subset of α-helix partners, including E6AP, the ubiquitin ligase that mediates E6-dependent degradation of the p53 protein.

Cassetti M C et al., Vaccine 22:520-52, 2004, examined the effects of mutations four or five amino acid positions in E6 and E7 to inactivate their oncogenic potential. The following mutations were examined: E6-C⁶³G and E6 C¹⁰⁶G (positions based on the wild type E6); E7-C²⁴G, E7-E²⁶G, and E7 C⁹¹G (positions based on the wild type E7). Venezuelan equine encephalitis virus replicon particle (VRP) vaccines encoding mutant or wild type E6 and E7 proteins elicited comparable CTL responses and generated comparable antitumor responses in several HPV16 E6(+)E7(+) tumor challenge models: protection from either C3 or TC-1 tumor challenge was observed in 100% of vaccinated mice. Eradication of C3 tumors was observed in approximately 90% of the mice. The predicted inactivation of E6 and E7 oncogenic potential was confirmed by demonstrating normal levels of both p53 and Rb proteins in human mammary epithelial cells infected with VRPs expressing mutant E6 and E7 genes.

The HPV16 E6 protein contains two zinc fingers important for structure and function; one cysteine (C) amino acid position in each pair of C—X—X—C (where X is any amino acid) zinc finger motifs may be mutated at E6 positions 63 and 106 (based on the wild type E6). Mutants are created, for example, using the Quick Change Site-Directed Mutagenesis Kit (Stratagene, La Jolla, Calif.). HPV16 E6 containing a single point mutation in the codon for Cys¹⁰⁶ in the wild type E6 (=Cys 113 in the wild type E6). Cys¹⁰⁶ neither binds nor facilitates degradation of p53 and is incapable of immortalizing human mammary epithelial cells (MEC), a phenotype dependent upon p53 degradation. A single amino acid substitution at position Cys⁶³ of the wild type E6 (=Cys⁷° in the wild type E6) destroys several HPV16 E6 functions: p53 degradation, E6TP-1 degradation, activation of telomerase, and, consequently, immortalization of primary epithelial cells.

Any nucleotide sequence that encodes these E6 polypeptides, one of the mutants thereof, or an antigenic fragment or epitope thereof, can be used in the present invention.

Other mutations can be tested and used in accordance with the methods described herein including those described in Cassetti et al., supra. These mutations can be produced from any appropriate starting sequences by mutation of the coding DNA.

The present invention also includes the use of a tandem E6-E7 vaccine, using one or more of the mutations described herein to render the oncoproteins inactive with respect to their oncogenic potential in vivo. VRP vaccines (described in Cassetti et al., supra) comprised fused E6 and E7 genes in one open reading frame which were mutated at four or five amino acid positions. Thus, the present constructs may include one or more epitopes of E6 and E7, which may be arranged in their native order or shuffled in any way that permits the expressed protein to bear the E6 and E7 antigenic epitopes in an immunogenic form. DNA encoding amino acid spacers between E6 and E7 or between individual epitopes of these proteins may be introduced into the vector, provided again, that the spacers permit the expression or presentation of the epitopes in an immunogenic manner after they have been expressed by transduced host cells.

### Influenza Hemagglutinin (HA)

A nucleic acid sequence encoding HA is shown herein as SEQ ID NO: 7. The amino acid sequence of HA is shown herein as SEQ ID NO: 8, with the immunodominant epitope underscored.

### Ovalbumin (OVA)

An amino acid sequence encoding a representative OVA is shown herein as SEQ ID NO:9.

### Other Exemplary Antigens

Exemplary antigens are epitopes of pathogenic microorganisms against which the host is defended by effector T cells responses, including CTL and delayed type hypersensitivity. These typically include viruses, intracellular parasites such as malaria, and bacteria that grow intracellularly such as Mycobacterium and Listeria species. Thus, the types of antigens included in the vaccine compositions used in the present invention may be any of those associated with such pathogens as well as tumor-specific antigens. It is noteworthy that some viral antigens are also tumor antigens in the case where the virus is a causative factor in the tumor.

In fact, the two most common cancers worldwide, hepatoma and cervical cancer, are associated with viral infection. Hepatitis B virus (HBV) (Beasley, R. P. et al., Lancet 2:1129-1133 (1981) has been implicated as etiologic agent of hepatomas. About 80-90% of cervical cancers express the E6 and E7 antigens (discussed above and exemplified herein) from one of four “high risk” human papillomavirus types: HPV-16, HPV-18, HPV-31 and HPV-45 (Gissmann, L. et al., Ciba Found Symp. 120:190-207, 1986; Beaudenon, S., et al. Nature 321:246-9, 1986, incorporated by reference herein). The HPV E6 and E7 antigens are the most promising targets for virus associated cancers in immunocompetent individuals because of their ubiquitous expression in cervical cancer. In addition to their importance as targets for therapeutic cancer vaccines, virus-associated tumor antigens are also ideal candidates for prophylactic vaccines. Indeed, introduction of prophylactic HBV vaccines in Asia have decreased the incidence of hepatoma (Chang, M H et al. New Engl. J. Med. 336, 1855-1859 (1997), representing a great impact on cancer prevention.

Among the most important viruses in chronic human viral infections are HPV, HBV, hepatitis C Virus (HCV), retroviruses such as human immunodeficiency virus (HIV-1 and HIV-2), herpes viruses such as Epstein Barr Virus (EBV), cytomegalovirus (CMV), HSV-1 and HSV-2, and influenza virus. Useful antigens include HBV surface antigen or HBV core antigen; ppUL83 or pp 89 of CMV; antigens of gp120, gp41 or p24 proteins of HIV-1; ICP27, gD2, gB of HSV; or influenza hemagglutinin or nucleoprotein (Anthony, L S et al., Vaccine 1999; 17:373-83). Other antigens associated with pathogens that can be utilized as described herein are antigens of various parasites, including malaria, e.g., malaria peptide based on repeats of NANP.

In certain embodiments, the invention includes methods using foreign antigens in which individuals may have existing T cell immunity (such as influenza, tetanus toxin, herpes etc). In other embodiments, the skilled artisan would readily be able to determine whether a subject has existing T cell immunity to a specific antigen according to well known methods available in the art and use a foreign antigen to which the subject does not already have an existing T cell immunity.

In alternative embodiments, the antigen is from a pathogen that is a bacterium, such as Bordetella pertussis; Ehrlichia chaffeensis; Staphylococcus aureus; Toxoplasma gondii; Legionella pneumophila; Brucella suis; Salmonella enterica; Mycobacterium avium; Mycobacterium tuberculosis; Listeria monocytogenes; Chlamydia trachomatis; Chlamydia pneumoniae; Rickettsia rickettsii; or, a fungus, such as, e.g., Paracoccidioides brasiliensis; or other pathogen, e.g., Plasmodium falciparum.

As used herein, the term “cancer” includes, but is not limited to, solid tumors and blood borne tumors. The term cancer includes diseases of the skin, tissues, organs, bone, cartilage, blood and vessels. A term used to describe cancer that is far along in its growth, also referred to as “late stage cancer” or “advanced stage cancer,” is cancer that is metastatic, e.g., cancer that has spread from its primary origin to another part of the body. In certain embodiments, advanced stage cancer includes stages 3 and 4 cancers. Cancers are ranked into stages depending on the extent of their growth and spread through the body; stages correspond with severity. Determining the stage of a given cancer helps doctors to make treatment recommendations, to form a likely outcome scenario for what will happen to the patient (prognosis), and to communicate effectively with other doctors.

There are multiple staging scales in use. One of the most common ranks cancers into five progressively more severe stages: 0, I, II, III, and IV. Stage 0 cancer is cancer that is just beginning, involving just a few cells. Stages I, II, III, and IV represent progressively more advanced cancers, characterized by larger tumor sizes, more tumors, the aggressiveness with which the cancer grows and spreads, and the extent to which the cancer has spread to infect adjacent tissues and body organs.

Another popular staging system is known as the TNM system, a three dimensional rating of cancer extensiveness. Using the TNM system, doctors rate the cancers they find on each of three scales, where T stands for tumor size, N stands for lymph node involvement, and M stands for metastasis (the degree to which cancer has spread beyond its original locations). Larger scores on each of the three scales indicate more advanced cancer. For example, a large tumor that has not spread to other body parts might be rated T3, N0, M0, while a smaller but more aggressive cancer might be rated T2, N2, M1 suggesting a medium sized tumor that has spread to local lymph nodes and has just gotten started in a new organ location.

Cancers that may be treated by the methods of the present invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In addition to its applicability to human cancer and infectious diseases, the present invention is also intended for use in treating animal diseases in the veterinary medicine context. Thus, the approaches described herein may be readily applied by one skilled in the art for treatment of veterinary herpes virus infections including equine herpes viruses, bovine viruses such as bovine viral diarrhea virus (for example, the E2 antigen), bovine herpes viruses, Marek's disease virus in chickens and other fowl; animal retroviral and lentiviral diseases (e.g., feline leukemia, feline immunodeficiency, simian immunodeficiency viruses, etc.); pseudorabies and rabies; and the like.

As for tumor antigens, any tumor-associated or tumor-specific antigen (or tumor cell derived epitope) (collectively, TAA) that can be recognized by T cells, including CTL, can be used. These include, without limitation, mutant p53, HER2/neu or a peptide thereof, or any of a number of melanoma-associated antigens such as MAGE-1, MAGE-3, MART-1/Melan-A, tyrosinase, gp75, gp100, BAGE, GAGE-1, GAGE-2, GnT-V, and p15 (see, for example, U.S. Pat. No. 6,187,306, incorporated herein by reference).

In one embodiment, it is not necessary to include a full length antigen in a nucleic acid vaccine; it suffices to include a fragment that will be presented by MHC class I and/or II. A nucleic acid may include 1, 2, 3, 4, 5 or more antigens, which may be the same or different ones.

Approaches for Mutagenesis of E6, E7, and other Antigens

Mutants of the antigens described here may be created, for example, using the Quick Change Site-Directed Mutagenesis Kit (Stratagene, La Jolla, Calif.). Generally, antigens that may be used herein may be proteins or peptides that differ from the naturally-occurring proteins or peptides but yet retain the necessary epitopes for functional activity. In certain embodiments, an antigen may comprise, consist essentially of, or consist of an amino acid sequence that is at least about 50%, 55%, 60%, 65%, 70%, 75%, 80%, 85%, 90%, 95%, 96%, 97%, 98%, or 99% identical to that of the naturally-occurring antigen or a fragment thereof. In certain embodiments, an antigen may also comprise, consist essentially of, or consist of an amino acid sequence that is encoded by a nucleotide sequence that is at least about 50%, 55%, 60%, 65%, 70%, 75%, 80%, 85%, 90%, 95%, 96%, 97%, 98%, or 99% identical to a nucleotide sequence encoding the naturally-occurring antigen or a fragment thereof. In certain embodiments, an antigen may also comprise, consist essentially of, or consist of an amino acid sequence that is encoded by a nucleic acid that hybridizes under high stringency conditions to a nucleic acid encoding the naturally-occurring antigen or a fragment thereof. Hybridization conditions are further described herein.

In one embodiment, an exemplary protein may comprise, consist essentially of, or consist of, an amino acid sequence that is at least about 50%, 55%, 60%, 65%, 70%, 75%, 80%, 85%, 90%, 95%, 96%, 97%, 98%, or 99% identical to that of a viral protein, including for example E6 or E7, such as an E6 or E7 sequence provided herein. Where the E6 or E7 protein is a detox E6 or E7 protein, the amino acid sequence of the protein may comprise, consist essentially of, or consist of an amino acid sequence that is at least about 50%, 55%, 60%, 65%, 70%, 75%, 80%, 85%, 90%, 95%, 96%, 97%, 98%, or 99% identical to that of an E6 or E7 protein, wherein the amino acids that render the protein a “detox” protein are present.

### Exemplary Nucleic Acid (e.g., DNA) Vaccines Encoding an Immunogenicity-Potentiating Polypeptide (IPP) and an Antigen

In one embodiment, a nucleic acid vaccine encodes a fusion protein comprising an antigen and a second protein, e.g., an IPP. An IPP may act in potentiating an immune response by promoting: processing of the linked antigenic polypeptide via the MHC class I pathway or targeting of a cellular compartment that increases the processing. This basic strategy may be combined with an additional strategy pioneered by the present inventors and colleagues, that involve linking DNA encoding another protein, generically termed a “targeting polypeptide,” to the antigen-encoding DNA. Again, for the sake of simplicity, the DNA encoding such a targeting polypeptide will be referred to herein as a “targeting DNA.” That strategy has been shown to be effective in enhancing the potency of the vectors carrying only antigen-encoding DNA. See for example, the following PCT publications by Wu et al: WO 01/29233; WO 02/009645; WO 02/061113; WO 02/074920; and WO 02/12281, all of which are incorporated by reference in their entirety. The other strategies include the use of DNA encoding polypeptides that promote or enhance:
(a) development, accumulation or activity of antigen presenting cells or targeting of antigen to compartments of the antigen presenting cells leading to enhanced antigen presentation;
(b) intercellular transport and spreading of the antigen;
(c) sorting of the lysosome-associated membrane protein type 1 (Sig/LAMP-1); or
(d) any combination of (a)-(c).

The strategy includes use of:
(a) a viral intercellular spreading protein selected from the group of herpes simplex virus-1 VP22 protein, Marek's disease virus UL49 (see WO 02/09645 and U.S. Pat. No. 7,318,928), protein or a functional homologue or derivative thereof;
(b) calreticulin (CRT) and other endoplasmic reticulum chaperone polypeptides selected from the group of CRT-like molecules ER60, GRP94, gp96, or a functional homologue or derivative thereof (see WO 02/12281 and U.S. Pat. No. 7,3442,002);
(c) a cytoplasmic translocation polypeptide domains of a pathogen toxin selected from the group of domain II of Pseudomonas exotoxin ETA or a functional homologue or derivative thereof (see published US application 20040086845);
(d) a polypeptide that targets the centrosome compartment of a cell selected from γ-tubulin or a functional homologue or derivative thereof;
(e) a polypeptide that stimulates dendritic cell precursors or activates dendritic cell activity selected from the group of GM-CSF, Flt3-ligand extracellular domain, or a functional homologue or derivative thereof;
(f) a costimulatory signal, such as a B7 family protein, including B7-DC (see U.S. Ser. No. 09/794,210), B7.1, B7.2, soluble CD40, etc.); or
(g) an anti-apoptotic polypeptide selected from the group consisting of (1) BCL-xL, (2) BCL2, (3) XIAP, (4) FLICEc-s, (5) dominant-negative caspase-8, (6) dominant negative caspase-9, (7) SPI-6, and (8) a functional homologue or derivative of any of (1)-(7). (See WO 2005/047501).

The following publications, all of which are incorporated by reference in their entirety, describe IPPs: Kim T W et al., J Clin Invest 112: 109-117, 2003; Cheng W F et al., J Clin Invest 108: 669-678, 2001; Hung C F et al., Cancer Res 61:3698-3703, 2001; Chen CH et al., 2000, supra; U.S. Pat. No. 6,734,173; published patent applications WO05/081716, WO05/047501, WO03/085085, WO02/12281, WO02/074920, WO02/061113, WO02/09645, and WO01/29233. Comparative studies of these IPPs using HPV E6 as the antigen are described in Peng, S. et al., J Biomed Sci. 12:689-700 2005.

An antigen may be linked N-terminally or C-terminally to an IPP. Exemplary IPPs and fusion constructs encoding such are described below.

### Lysosomal Associated Membrane Protein 1 (LAMP-1)

The DNA sequence encoding the E7 protein fused to the translocation signal sequence and LAMP-1 domain (Sig-E7-LAMP-1) is shown herein as SEQ ID NO:10. The amino acid sequence of Sig-E7-LAMP-1 is shown herein as SEQ ID NO:11.

The nucleotide sequence of the immunogenic vector pcDNA3-Sig/E7/LAMP-1 is shown herein as SEQ ID NO:13, with the SigE7-LAMP-1 coding sequence in lower case and underscored.

HSP70 from M. tuberculosis

The nucleotide sequence encoding HSP70 is shown herein as SEQ ID NO:13) (i.e., nucleotides 10633-12510 of the M. tuberculosis genome in GenBank NC_{—}000962). The amino acid sequence of HSP70 is shown herein as SEQ ID NO:14.

The nucleic acid sequences encoding the E7-Hsp70 chimera/fusion polypeptides are shown herein as SEQ ID NO:15 and the corresponding amino acid sequence is shown herein as SEQ ID NO:16. The E7 coding sequence is shown in upper case and underscored.

ETA(dII) from Pseudomonas aeruginosa

The complete coding sequence for Pseudomonas aeruginosa exotoxin type A (ETA) is shown herein as SEQ ID NO:17 (GenBank Accession No. K01397). The amino acid sequence of ETA is shown herein as SEQ ID NO:18 (GenBank Accession No. K01397).

Residues 1-25 (italicized) represent the signal peptide. The first residue of the mature polypeptide, Ala, is bolded/underscored. The mature polypeptide is residues 26-638 of SEQ ID NO:18.

Domain II (ETA(II)), translocation domain (underscored above) spans residues 247-417 of the mature polypeptide (corresponding to residues 272-442 of SEQ ID NO:18) and is presented below separately herein as SEQ ID NO:19.

The nucleotide construct in which ETA(dII) is fused to HPV-16 E7 is shown herein as SEQ ID NO:20. The corresponding amino acid sequence is shown herein as SEQ ID NO:21. The ETA(dII) sequence appears in plain font, extra codons from plasmid pcDNA3 are italicized. Nucleotides between ETA(dII) and E7 are also bolded (and result in the interposition of two amino acids between ETA(dII) and E7). The E7 amino acid sequence is underscored (ends with Gln at position 269).

### Pro Leu Ile Ser Leu Asp Cys Ala Phe AMB

The nucleotide sequence of the pcDNA3 vector encoding E7 and HSP70 (pcDNA3-E7-Hsp70 is shown herein as SEQ ID NO:22.

### Calreticulin (CRT)

Calreticulin (CRT), a well-characterized ˜46 kDa protein was described briefly above, as were a number of its biological and biochemical activities. As used herein, “calreticulin” or “CRT” refers to polypeptides and nucleic acids molecules having substantial identity to the exemplary human CRT sequences as described herein or homologues thereof, such as rabbit and rat CRT—well-known in the art. A CRT polypeptide is a polypeptide comprising a sequence identical to or substantially identical to the amino acid sequence of CRT. An exemplary nucleotide and amino acid sequence for a CRT used in the present compositions and methods are presented below. The terms “calreticulin” or “CRT” encompass native proteins as well as recombinantly produced modified proteins that, when fused with an antigen (at the DNA or protein level) promote the induction of immune responses and promote angiogenesis, including a CTL response. Thus, the terms “calreticulin” or “CRT” encompass homologues and allelic variants of human CRT, including variants of native proteins constructed by in vitro techniques, and proteins isolated from natural sources. The CRT polypeptides used in the present invention, and sequences encoding them, also include fusion proteins comprising non-CRT sequences, particularly MHC class I-binding peptides; and also further comprising other domains, e.g., epitope tags, enzyme cleavage recognition sequences, signal sequences, secretion signals and the like.

A human CRT coding sequence is shown herein as SEQ ID NO: 23. The amino acid sequence of the human CRT protein encoded by SEQ ID NO:23 is set forth herein as SEQ ID NO:24. This amino acid sequence is highly homologous to GenBank Accession No. NM 004343.

The amino acid sequence of the rabbit and rat CRT proteins are set forth in GenBank Accession Nos. P1553 and NM 022399, respectively. An alignment of human, rabbit and rat CRT shows that these proteins are highly conserved, and most of the amino acid differences between species are conservative in nature. Most of the variation is found in the alignment of the approximately 36 C-terminal residues. Thus, for the present invention, human CRT may be used as well as, DNA encoding any homologue of CRT from any species that has the requisite biological activity (as an IPP) or any active domain or fragment thereof, may be used in place of human CRT or a domain thereof.

Cheng et al., supra, incorporated by reference in its entirety, previously determined that nucleic acid (e.g., DNA) vaccines encoding each of the N, P, and C domains of CRT chimerically linked to HPV-16 E7 elicited potent antigen-specific CD8+ T cell responses and antitumor immunity in mice vaccinated i.d., by gene gun administration. N-CRT/E7, P-CRT/E7 or C-CRT/E7 DNA each exhibited significantly increased numbers of E7-specific CD8⁺ T cell precursors and impressive antitumor effects against E7-expressing tumors when compared with mice vaccinated with E7 DNA (antigen only). N-CRT DNA administration also resulted in anti-angiogenic antitumor effects. Thus, cancer therapy using DNA encoding N-CRT linked to a tumor antigen may be used for treating tumors through a combination of antigen-specific immunotherapy and inhibition of angiogenesis.

The constructs comprising CRT or one of its domains linked to E7 is illustrated schematically below.

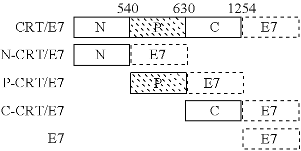

The amino acid sequences of the 3 human CRT domains are shown herein as annotations of the full length protein, SEQ ID NO:24. The N domain comprises residues 1-170 (normal text); the P domain comprises residues 171-269 (underscored); and the C domain comprises residues 270-417 (bold/italic).

The sequences of the three domains are further shown as separate polypeptides herein as human N-CRT (SEQ ID NO:25), as human P-CRT (SEQ ID NO:26), and as human C-CRT (SEQ ID NO:27).

The present vectors may comprises DNA encoding one or more of these domain sequences, which are shown by annotation of SEQ ID NO:28 herein, wherein the N-domain sequence is upper case, the P-domain sequence is lower case/italic/underscored, and the C domain sequence is lower case. The stop codon is also shown but not counted.

The coding sequence for each separate domain is provided herein as human N-CRT DNA (SEQ ID NO:29), as human P-CRT DNA (SEQ ID NO:30), and as human C-CRT DNA (SEQ ID NO:31). Alternatively, any nucleotide sequences that encodes these domains may be used in the present constructs. Thus, for use in humans, the sequences may be further codon-optimized.

Constructs used in the present invention may employ combinations of one or more CRT domains, in any of a number of orientations. Using the designations N^{CRT}, P^{CRT} and C^{CRT} to designate the domains, the following are but a few examples of the combinations that may be used in the nucleic acid (e.g., DNA) vaccine vectors used in the present invention (where it is understood that Ag can be any antigen, including E7(detox) or E6 (detox).

| | | | |
|---|---|---|---|
| N^{CRT}-P^{CRT}-Ag; | N^{CRT}-P^{CRT}-Ag; | N^{CRT}-C^{CRT}-Ag; | N^{CRT}-N^{CRT}-Ag; |
| N^{CRT}-N^{CRT}-N^{CRT}-Ag; | P^{CRT}-P^{CRT}-Ag; | P^{CRT}-C^{CRT}-Ag; | P^{CRT}-N^{CRT}-Ag; |
| C^{CRT}-P^{CRT}-Ag; | N^{CRT}-P^{CRT}-Ag; | etc. | |

The present invention may employ shorter polypeptide fragments of CRT or CRT domains provided such fragments can enhance the immune response to an antigen with which they are paired. Shorter peptides from the CRT or domain sequences shown above that have the ability to promote protein processing via the MHC-1 class I pathway are also included, and may be defined by routine experimentation.

The present invention may also employ shorter nucleic acid fragments that encode CRT or CRT domains provided such fragments are functional, e.g., encode polypeptides that can enhance the immune response to an antigen with which they are paired (e.g., linked). Nucleic acids that encode shorter peptides from the CRT or domain sequences shown above and are functional, e.g., have the ability to promote protein processing via the MHC-1 class I pathway, are also included, and may be defined by routine experimentation.

A polypeptide fragment of CRT may include at least or about 50, 100, 200, 300, or 400 amino acids. A polypeptide fragment of CRT may also include at least or about 25, 50, 75, 100, 25-50, 50-100, or 75-125 amino acids from a CRT domain selected from the group N-CRT, P-CRT, and C-CRT. A polypeptide fragment of CRT may include residues 1-50, 50-75, 75-100, 100-125, 125-150, 150-170 of the N-domain (e.g., of SEQ ID NO:25). A polypeptide fragment of CRT may include residues 1-50, 50-75, 75-100, 100-109 of the P-domain (e.g., of SEQ ID NO:26). A polypeptide fragment of CRT may include residues 1-50, 50-75, 75-100, 100-125, 125-138 of the C-domain (e.g., of SEQ ID NO:27).

A nucleic acid fragment of CRT may encode at least or about 50, 100, 200, 300, or 400 amino acids. A nucleic acid fragment of CRT may also encode at least or about 25, 50, 75, 100, 25-50, 50-100, or 75-125 amino acids from a CRT domain selected from the group N-CRT, P-CRT, and C-CRT. A nucleic acid fragment of CRT may encode residues 1-50, 50-75, 75-100, 100-125, 125-150, 150-170 of the N-domain (e.g., of SEQ ID NO:25). A nucleic acid fragment of CRT may encode residues 1-50, 50-75, 75-100, 100-109 of the P-domain (e.g., of SEQ ID NO:26). A nucleic acid fragment of CRT may encode residues 1-50, 50-75, 75-100, 100-125, 125-138 of the C-domain (e.g., of SEQ ID NO:27).

Polypeptide “fragments” of CRT, as provided herein, do not include full-length CRT. Likewise, nucleic acid “fragments” of CRT, as provided herein, do not include a full-length CRT nucleic acid sequence and do not encode a full-length CRT polypeptide.

In one embodiment, a vector construct of a complete chimeric nucleic acid that can be used in the present invention, is shown herein as SEQ ID NO:32. The sequence is annotated to show plasmid-derived nucleotides (lower case letters), CRT-derived nucleotides (upper case bold letters), and HPV-E7-derived nucleotides (upper case, italicized/underlined letters). Five plasmid nucleotides are found between the CRT and E7 coding sequences and that the stop codon for the E7 sequence is double underscored. This plasmid is also referred to as pNGVL4a-CRT/E7(detox). The Table below describes the structure of the above plasmid.

| Plasmid | | |
|---|---|---|
| Position | Genetic Construct | Source of Construct |
| 5970-0823 | E. coli ORI (ColEl) | pBR/E. coli-derived |
| 0837-0881 | portion of transposase (tpnA) | Common plasmid sequence |
| | | Tn5/Tn903 |
| 0882-1332 | β-Lactamase (Amp^{R}) | pBRpUC derived plasmid |
| 1331-2496 | AphA (Kan^{R}) | Tn903 |
| 2509-2691 | P3 Promoter DNA binding | Tn3/pBR322 |
| | site | |
| 2692-2926 | pUC backbone | Common plasmid sequence |
| | | pBR322-derived |
| 2931-4009 | NF1 binding and promoter | HHV-5(HCMV UL-10 lE1 |
| | | gene) |
| 4010-4014 | Poly-cloning site | Common plasmid sequence |
| 4015-5265 | Calreticulin (CRT) | Human Calreticulin |
| 5266-5271 | GAATTC plasmid sequence | Remain after cloning |
| 5272-5568 | dE7 gene (detoxified | HPV-16 (E7 gene) incl. stop |
| | partial) | codon |
| 5569-5580 | Poly-cloning site | Common plasmid sequence |
| 551-5970 | Poly-Adenylation site | Mammalian signal, pHCMV- |
| | | derived |

In some embodiments, an alternative to CRT is another ER chaperone polypeptide exemplified by ER60, GRP94 or gp96, well-characterized ER chaperone polypeptide that representatives of the HSP90 family of stress-induced proteins (see WO 02/012281, incorporated herein by reference). The term “endoplasmic reticulum chaperone polypeptide” as used herein means any polypeptide having substantially the same ER chaperone function as the exemplary chaperone proteins CRT, tapasin, ER60 or calnexin. Thus, the term includes all functional fragments or variants or mimics thereof. A polypeptide or peptide can be routinely screened for its activity as an ER chaperone using assays known in the art. While the present invention is not limited by any particular mechanism of action, in vivo chaperones promote the correct folding and oligomerization of many glycoproteins in the ER, including the assembly of the MHC class I heterotrimeric molecule (heavy (H) chain, β2m, and peptide). They also retain incompletely assembled MHC class I heterotrimeric complexes in the ER (Hauri FEBS Lett. 476:32-37, 2000).

### Intercellular Spreading Proteins

The potency of naked nucleic acid (e.g., DNA) vaccines may be enhanced by their ability to amplify and spread in vivo. VP22, a herpes simplex virus type 1 (HSV-1) protein and its “homologues” in other herpes viruses, such as the avian Marek's Disease Virus (MDV) have the property of intercellular transport that provide an approach for enhancing vaccine potency. The present inventors have previously created novel fusions of VP22 with a model antigen, human papillomavirus type 16 (HPV-16) E7, in a nucleic acid (e.g., DNA) vaccine which generated enhanced spreading and MHC class I presentation of antigen. These properties led to a dramatic increase in the number of E7-specific CD8+ T cell precursors in vaccinated mice (at least 50-fold) and converted a less effective nucleic acid (e.g., DNA) vaccine into one with significant potency against E7-expressing tumors. In comparison, a non-spreading mutant, VP22(1-267), failed to enhance vaccine potency. Results presented in U.S. Patent Application publication No. 20040028693 (U.S. Pat. No. 7,318,928), hereby incorporated by reference in its entirety, show that the potency of DNA vaccines is dramatically improved through enhanced intercellular spreading and MHC class I presentation of the antigen.

A similar study linking MDV-1 UL49 to E7 also led to a dramatic increase in the number of E7-specific CD8+ T cell precursors and potency response against E7-expressing tumors in vaccinated mice. Mice vaccinated with a MDV-1 UL49 DNA vaccine stimulated E7-specific CD8+ T cell precursor at a level comparable to that induced by HSV-1 VP22/E7. Thus, fusion of MDV-1UL49 DNA to DNA encoding a target antigen gene significantly enhances the DNA vaccine potency.

In one embodiment, the spreading protein may be a viral spreading protein, including a herpes virus VP22 protein. Exemplified herein are fusion constructs that comprise herpes simplex virus-1 (HSV-1) VP22 (abbreviated HVP22) and its homologue from Marek's disease virus (MDV) termed MDV-VP22 or MVP-22. Also included in the invention are the use of homologues of VP22 from other members of the herpesviridae or polypeptides from nonviral sources that are considered to be homologous and share the functional characteristic of promoting intercellular spreading of a polypeptide or peptide that is fused or chemically conjugated thereto.

DNA encoding HVP22 has the sequence SEQ ID NO:33 of the longer sequence SEQ ID NO:34 (which is the full length nucleotide sequence of a vector that comprises HVP22). DNA encoding MDV-VP22 is shown herein as SEQ ID NO:35.

The amino acid sequence of HVP22 polypeptide is SEQ ID NO:36 as amino acid residues 1-301 of SEQ ID NO:37 (i.e., the full length amino acid encoded by the vector).

The amino acid sequence of the MDV-VP22 is shown herein as SEQ ID NO:38.

A DNA clone pcDNA3 VP22/E7, that includes the coding sequence for HVP22 and the HPV-16 protein, E7 (plus some additional vector sequence) is SEQ ID NO:34.

The amino acid sequence of E7 (SEQ ID NO:39) is residues 308-403 of SEQ ID NO:37. This particular clone has only 96 of the 98 residues present in E7. The C-terminal residues of wild-type E7, Lys and Pro, are absent from this construct. This is an example of a deletion variant as the term is described below. Such deletion variants (e.g., terminal truncation of two or a small number of amino acids) of other antigenic polypeptides are examples of the embodiments intended within the scope of the fusion polypeptides that can be used in the present invention.

### Homologues of IPPs

Homologues or variants of IPPs described herein, may also be used, provided that they have the requisite biological activity. These include various substitutions, deletions, or additions of the amino acid or nucleic acid sequences. Due to code degeneracy, for example, there may be considerable variation in nucleotide sequences encoding the same amino acid sequence.

A functional derivative of an IPP retains measurable IPP-like activity, including that of promoting immunogenicity of one or more antigenic epitopes fused thereto by promoting presentation by class I pathways. “Functional derivatives” encompass “variants” and “fragments” regardless of whether the terms are used in the conjunctive or the alternative herein.

The term “chimeric” or “fusion” polypeptide or protein refers to a composition comprising at least one polypeptide or peptide sequence or domain that is chemically bound in a linear fashion with a second polypeptide or peptide domain. One embodiment of compositions useful for the present invention is an isolated or recombinant nucleic acid molecule encoding a fusion protein comprising at least two domains, wherein the first domain comprises an IPP and the second domain comprises an antigenic epitope, e.g., an MHC class I-binding peptide epitope. The “fusion” can be an association generated by a peptide bond, a chemical linking, a charge interaction (e.g., electrostatic attractions, such as salt bridges, H-bonding, etc.) or the like. If the polypeptides are recombinant, the “fusion protein” can be translated from a common mRNA. Alternatively, the compositions of the domains can be linked by any chemical or electrostatic means. The chimeric molecules that can be used in the present invention (e.g., targeting polypeptide fusion proteins) can also include additional sequences, e.g., linkers, epitope tags, enzyme cleavage recognition sequences, signal sequences, secretion signals, and the like. Alternatively, a peptide can be linked to a carrier simply to facilitate manipulation or identification/location of the peptide.

Also included is a “functional derivative” of an IPP, which refers to an amino acid substitution variant, a “fragment” of the protein. A functional derivative of an IPP retains measurable activity that may be manifested as promoting immunogenicity of one or more antigenic epitopes fused thereto or co-administered therewith. “Functional derivatives” encompass “variants” and “fragments” regardless of whether the terms are used in the conjunctive or the alternative herein.

A functional homologue must possess the above biochemical and biological activity. In view of this functional characterization, use of homologous proteins including proteins not yet discovered, fall within the scope of the invention if these proteins have sequence similarity and the recited biochemical and biological activity.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the method of alignment includes alignment of Cys residues.

In one embodiment, the length of a sequence being compared is at least 30%, at least 40%, at least 50%, at least 60%, and at least 70%, 80%, 90%, 95%, 96%, 97%, 98%, or 99% of the length of the reference sequence (e.g., an IPP). The amino acid residues (or nucleotides) at corresponding amino acid (or nucleotide) positions are then compared. When a position in the first sequence is occupied by the same amino acid residue (or nucleotide) as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In one embodiment, the percent identity between two amino acid sequences is determined using the Needleman and Wunsch (J. Mol. Biol. 48:444-453 (1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases, for example, to identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to IPP nucleic acid molecules. BLAST protein searches can be performed with the XBLAST program, score=50, wordlength=3 to obtain amino acid sequences homologous to IPP protein molecules. To obtain gapped alignments for comparison purposes, Gapped BLAST can be utilized as described in Altschul et al. (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

Thus, a homologue of an IPP or of an IPP domain described above is characterized as having (a) functional activity of native IPP or domain thereof and (b) amino acid sequence similarity to a native IPP protein or domain thereof when determined as above, of at least about 50%, 55%, 60%, 65%, 70%, 75%, 80%, 85%, 90%, 95%, 96%, 97%, 98%, or 99%.

It is within the skill in the art to obtain and express such a protein using DNA probes based on the disclosed sequences of an IPP. Then, the fusion protein's biochemical and biological activity can be tested readily using art-recognized methods such as those described herein, for example, a T cell proliferation, cytokine secretion or a cytolytic assay, or an in vivo assay of tumor protection or tumor therapy. A biological assay of the stimulation of antigen-specific T cell reactivity will indicate whether the homologue has the requisite activity to qualify as a “functional” homologue.

A “variant” refers to a molecule substantially identical to either the full protein or to a fragment thereof in which one or more amino acid residues have been replaced (substitution variant) or which has one or several residues deleted (deletion variant) or added (addition variant). A “fragment” of an IPP refers to any subset of the molecule, that is, a shorter polypeptide of the full-length protein.

A number of processes can be used to generate fragments, mutants and variants of the isolated DNA sequence. Small subregions or fragments of the nucleic acid encoding the spreading protein, for example 1-30 bases in length, can be prepared by standard, chemical synthesis. Antisense oligonucleotides and primers for use in the generation of larger synthetic fragment.

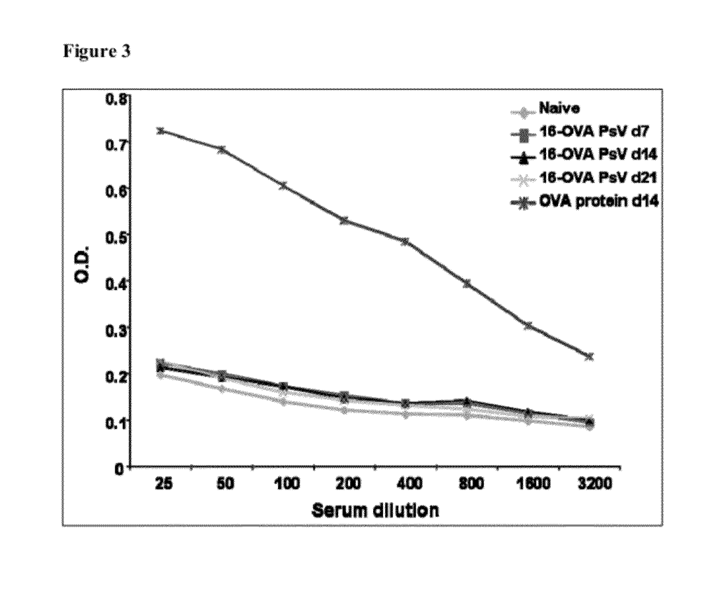

A one group of variants are those in which at least one amino acid residue and in certain embodiments only one, has been substituted by different residue. For a detailed description of protein chemistry and structure, see Schulz, G E et al., Principles of Protein Structure, Springer-Verlag, New York, 1978, and Creighton, T. E., Proteins: Structure and Molecular Properties, W.H. Freeman & Co., San Francisco, 1983, which are hereby incorporated by reference. The types of substitutions that may be made in the protein molecule may be based on analysis of the frequencies of amino acid changes between a homologous protein of different species, such as those presented in Table 1-2 of Schulz et al. (supra) and FIG. 3-9 of Creighton (supra). Based on such an analysis, conservative substitutions are defined herein as exchanges within one of the following five groups:

| | | |
|---|---|---|
| 1. | Small aliphatic, nonpolar or slightly polar | Ala, Ser, Thr (Pro, Gly); |
| | residues | |
| 2. | Polar, negatively charged residues and | Asp, Asn, Glu, Gln; |
| | their amides | |
| 3. | Polar, positively charged residues | His, Arg, Lys; |
| 4. | Large aliphatic, nonpolar residues | Met, Leu, Ile, Val (Cys) |
| 5. | Large aromatic residues | Phe, Tyr, Trp. |

The three amino acid residues in parentheses above have special roles in protein architecture. Gly is the only residue lacking a side chain and thus imparts flexibility to the chain. Pro, because of its unusual geometry, tightly constrains the chain. Cys can participate in disulfide bond formation, which is important in protein folding.

More substantial changes in biochemical, functional (or immunological) properties are made by selecting substitutions that are less conservative, such as between, rather than within, the above five groups. Such changes will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain. Examples of such substitutions are (i) substitution of Gly and/or Pro by another amino acid or deletion or insertion of Gly or Pro; (ii) substitution of a hydrophilic residue, e.g., Ser or Thr, for (or by) a hydrophobic residue, e.g., Leu, Ile, Phe, Val or Ala; (iii) substitution of a Cys residue for (or by) any other residue; (iv) substitution of a residue having an electropositive side chain, e.g., Lys, Arg or H is, for (or by) a residue having an electronegative charge, e.g., Glu or Asp; or (v) substitution of a residue having a bulky side chain, e.g., Phe, for (or by) a residue not having such a side chain, e.g., Gly.

Most acceptable deletions, insertions and substitutions according to the present invention are those that do not produce radical changes in the characteristics of the wild-type or native protein in terms of its relevant biological activity, e.g., its ability to stimulate antigen specific T cell reactivity to an antigenic epitope or epitopes that are fused to the protein. However, when it is difficult to predict the exact effect of the substitution, deletion or insertion in advance of doing so, one skilled in the art will appreciate that the effect can be evaluated by routine screening assays such as those described here, without requiring undue experimentation.

Exemplary fusion proteins provided herein comprise an IPP protein or homolog thereof and an antigen. For example, a fusion protein may comprise, consist essentially of, or consist of an IPP or an IPP fragment, e.g., N-CRT, P-CRT and/or C-CRT, or an amino acid sequence that is at least about 50%, 55%, 60%, 65%, 70%, 75%, 80%, 85%, 90%, 95%, 96%, 97%, 98%, or 99% identical to the amino acid sequence of the IPP or IPP fragment, wherein the IPP fragment is functionally active as further described herein, linked to an antigen. A fusion protein may also comprise an IPP or an IPP fragment and at least 1, 2, 3, 4, 5, 6, 7, 8, 9, 10 or more amino acids, or about 1-5,1-10, 1-15, 1-20, 1-25, 1-30, 1-50 amino acids, at the N- and/or C-terminus of the IPP fragment. These additional amino acids may have an amino acid sequence that is unrelated to the amino acid sequence at the corresponding position in the IPP protein.

Homologs of an IPP or an IPP fragments may also comprise, consist essentially of, or consist of an amino acid sequence that differs from that of an IPP or IPP fragment by the addition, deletion, or substitution, e.g., conservative substitution, of at least about 1, 2, 3, 4, 5, 6, 7, 8, 9 or 10 amino acids, or from about 1-5, 1-10, 1-15 or 1-20 amino acids. Homologs of an IPP or IPP fragments may be encoded by nucleotide sequences that are at least about 50%, 55%, 60%, 65%, 70%, 75%, 80%, 85%, 90%, 95%, 96%, 97%, 98%, or 99% identical to the nucleotide sequence encoding an IPP or IPP fragment, such as those described herein.

Yet other homologs of an IPP or IPP fragments are encoded by nucleic acids that hybridize under stringent hybridization conditions to a nucleic acid that encodes an IPP or IPP fragment. For example, homologs may be encoded by nucleic acids that hybridize under high stringency conditions of 0.2 to 1×SSC at 65° C. followed by a wash at 0.2×SSC at 65° C. to a nucleic acid consisting of a sequence described herein. Nucleic acids that hybridize under low stringency conditions of 6×SSC at room temperature followed by a wash at 2×SSC at room temperature to nucleic acid consisting of a sequence described herein or a portion thereof can be used. Other hybridization conditions include 3×SSC at 40 or 50° C., followed by a wash in 1 or 2×SSC at 20, 30, 40, 50, 60, or 65° C. Hybridizations can be conducted in the presence of formaldehyde, e.g., 10%, 20%, 30% 40% or 50%, which further increases the stringency of hybridization. Theory and practice of nucleic acid hybridization is described, e.g., in S. Agrawal (ed.) Methods in Molecular Biology, volume 20; and Tijssen (1993) Laboratory Techniques in biochemistry and molecular biology-hybridization with nucleic acid probes, e.g., part I chapter 2 “Overview of principles of hybridization and the strategy of nucleic acid probe assays,” Elsevier, New York provide a basic guide to nucleic acid hybridization.

A fragment of a nucleic acid sequence is defined as a nucleotide sequence having fewer nucleotides than the nucleotide sequence encoding the full length CRT polypeptide, antigenic polypeptide, or the fusion thereof. This invention includes the use of such nucleic acid fragments that encode polypeptides which retain the ability of the fusion polypeptide to induce increases in frequency or reactivity of T cells, including CD8+ T cells, that are specific for the antigen part of the fusion polypeptide.

Nucleic acid sequences that can be used in the present invention may also include linker sequences, natural or modified restriction endonuclease sites and other sequences that are useful for manipulations related to cloning, expression or purification of encoded protein or fragments. For example, a fusion protein may comprise a linker between the antigen and the IPP protein.

Other nucleic acid vaccines that may be used include single chain trimers (SCT), as further described in the Examples and in references cited therein, all of which are specifically incorporated by reference herein.

### Backbone of Nucleic Acid Vaccine

A nucleic acid, e.g., DNA vaccine may comprise an “expression vector” or “expression cassette,” i.e., a nucleotide sequence which is capable of affecting expression of a protein coding sequence in a host compatible with such sequences. Expression cassettes include at least a promoter operably linked with the polypeptide coding sequence; and, optionally, with other sequences, e.g., transcription termination signals. Additional factors necessary or helpful in effecting expression may also be included, e.g., enhancers.

“Operably linked” means that the coding sequence is linked to a regulatory sequence in a manner that allows expression of the coding sequence. Known regulatory sequences are selected to direct expression of the desired protein in an appropriate host cell. Accordingly, the term “regulatory sequence” includes promoters, enhancers and other expression control elements. Such regulatory sequences are described in, for example, Goeddel, Gene Expression Technology. Methods in Enzymology, vol. 185, Academic Press, San Diego, Calif. (1990)).

A promoter region of a DNA or RNA molecule binds RNA polymerase and promotes the transcription of an “operably linked” nucleic acid sequence. As used herein, a “promoter sequence” is the nucleotide sequence of the promoter which is found on that strand of the DNA or RNA which is transcribed by the RNA polymerase. Two sequences of a nucleic acid molecule, such as a promoter and a coding sequence, are “operably linked” when they are linked to each other in a manner which permits both sequences to be transcribed onto the same RNA transcript or permits an RNA transcript begun in one sequence to be extended into the second sequence. Thus, two sequences, such as a promoter sequence and a coding sequence of DNA or RNA are operably linked if transcription commencing in the promoter sequence will produce an RNA transcript of the operably linked coding sequence. In order to be “operably linked” it is not necessary that two sequences be immediately adjacent to one another in the linear sequence.

In one embodiment, certain promoter sequences useful for the present invention must be operable in mammalian cells and may be either eukaryotic or viral promoters. Certain promoters are also described in the Examples, and other useful promoters and regulatory elements are discussed below. Suitable promoters may be inducible, repressible or constitutive. A “constitutive” promoter is one which is active under most conditions encountered in the cell's environmental and throughout development. An “inducible” promoter is one which is under environmental or developmental regulation. A “tissue specific” promoter is active in certain tissue types of an organism. An example of a constitutive promoter is the viral promoter MSV-LTR, which is efficient and active in a variety of cell types, and, in contrast to most other promoters, has the same enhancing activity in arrested and growing cells. Other viral promoters include that present in the CMV-LTR (from cytomegalovirus) (Bashart, M. et al., Cell 41:521, 1985) or in the RSV-LTR (from Rous sarcoma virus) (Gorman, C. M., Proc. Natl. Acad. Sci. USA 79:6777, 1982). Also useful are the promoter of the mouse metallothionein I gene (Hamer, D, et al., J. Mol. Appl. Gen. 1:273-88, 1982; the TK promoter of Herpes virus (McKnight, S, Cell 31:355-65, 1982); the SV40 early promoter (Benoist, C., et al., Nature 290:304-10, 1981); and the yeast gal4 gene promoter (Johnston, S A et al., Proc. Natl. Acad. Sci. USA 79:6971-5, 1982); Silver, Pa., et al., Proc. Natl. Acad. Sci. (USA) 81:5951-5, 1984)). Other illustrative descriptions of transcriptional factor association with promoter regions and the separate activation and DNA binding of transcription factors include: Keegan et al., Nature 231:699, 1986; Fields et al., Nature 340:245, 1989; Jones, Cell 61:9, 1990; Lewin, Cell 61:1161, 1990; Ptashne et al., Nature 346:329, 1990; Adams et al., Cell 72:306, 1993.

The promoter region may further include an octamer region which may also function as a tissue specific enhancer, by interacting with certain proteins found in the specific tissue. The enhancer domain of the DNA construct useful for the present invention is one which is specific for the target cells to be transfected, or is highly activated by cellular factors of such target cells. Examples of vectors (plasmid or retrovirus) are disclosed, e.g., in Roy-Burman et al., U.S. Pat. No. 5,112,767, incorporated by reference. For a general discussion of enhancers and their actions in transcription, see, Lewin, B M, Genes IV, Oxford University Press pp. 552-576, 1990 (or later edition). Particularly useful are retroviral enhancers (e.g., viral LTR) that is placed upstream from the promoter with which it interacts to stimulate gene expression. For use with retroviral vectors, the endogenous viral LTR may be rendered enhancer-less and substituted with other desired enhancer sequences which confer tissue specificity or other desirable properties such as transcriptional efficiency.

Thus, expression cassettes include plasmids, recombinant viruses, any form of a recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include replicons (e.g., RNA replicons), bacteriophages) to which fragments of DNA may be attached and become replicated. Vectors thus include, but are not limited to RNA, autonomous self-replicating circular or linear DNA or RNA, e.g., plasmids, viruses, and the like (U.S. Pat. No. 5,217,879, incorporated by reference), and includes both the expression and nonexpression plasmids. Where a recombinant cell or culture is described as hosting an “expression vector” this includes both extrachromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

Exemplary virus vectors that may be used include recombinant adenoviruses (Horowitz, M S, In: Virology, Fields, B N et al., eds, Raven Press, NY, 1990, p. 1679; Berkner, K L, Biotechniques 6:616-29, 1988; Strauss, S E, In: The Adenoviruses, Ginsberg, H S, ed., Plenum Press, NY, 1984, chapter 11) and herpes simplex virus (HSV). Advantages of adenovirus vectors for human gene delivery include the fact that recombination is rare, no human malignancies are known to be associated with such viruses, the adenovirus genome is double stranded DNA which can be manipulated to accept foreign genes of up to 7.5 kb in size, and live adenovirus is a safe human vaccine organisms. Adeno-associated virus is also useful for human therapy (Samulski, R J et al., EMBO J. 10:3941, 1991) according to the present invention.

A nucleic acid (e.g., DNA) vaccine may also use a replicon, e.g., an RNA replicon, a self-replicating RNA vector. In one embodiment, a replicon is one based on a Sindbis virus RNA replicon, e.g., SINrepS. The present inventors tested E7 in the context of such a vaccine and showed (see Wu et al, U.S. patent application Ser. No. 10/343,719) that a Sindbis virus RNA vaccine encoding HSV-1 VP22 linked to E7 significantly increased activation of E7-specific CD8 T cells, resulting in potent antitumor immunity against E7-expressing tumors. The Sindbis virus RNA replicon vector used in these studies, SINrepS, has been described (Bredenbeek, P J et al., 1993, J. Virol. 67:6439-6446).

Generally, RNA replicon vaccines may be derived from alphavirus vectors, such as Sindbis virus (Hariharan, M J et al., 1998. J Virol 72:950-8.), Semliki Forest virus (Berglund, P M et al., 1997. AIDS Res Hum Retroviruses 13:1487-95; Ying, H T et al., 1999. Nat Med 5:823-7) or Venezuelan equine encephalitis virus (Pushko, P M et al., 1997. Virology 239:389-401). These self-replicating and self-limiting vaccines may be administered as either (1) RNA or (2) DNA which is then transcribed into RNA replicons in cells transfected in vitro or in vivo (Berglund, P C et al., 1998. Nat Biotechnol 16:562-5; Leitner, W W et al., 2000. Cancer Res 60:51-5). An exemplary Semliki Forest virus is pSCA1 (DiCiommo, D P et al., J Biol Chem 1998; 273:18060-6).

The plasmid vector pcDNA3 or a functional homolog thereof (SEQ ID NO:40) may be used in a nucleic acid (e.g., DNA) vaccine. In other embodiments, pNGVL4a (SEQ ID NO:41) can be used.

pNGVL4a, one plasmid backbone for use in the present invention, was originally derived from the pNGVL3 vector, which has been approved for human vaccine trials. The pNGVL4a vector includes two immunostimulatory sequences (tandem repeats of CpG dinucleotides) in the noncoding region. Whereas any other plasmid DNA that can transform either APCs, including DC's or other cells which, via cross-priming, transfer the antigenic moiety to DCs, is useful in the present invention, pNGFVLA4a may be used because of the fact that it has already been approved for human therapeutic use.

The following references set forth principles and current information in the field of basic, medical and veterinary virology and are incorporated by reference: Fields Virology, Fields, B N et al., eds., Lippincott Williams & Wilkins, N.Y., 1996; Principles of Virology: Molecular Biology, Pathogenesis, and Control, Flint, S. J. et al., eds., Amer Soc Microbiol, Washington D.C., 1999; Principles and Practice of Clinical Virology, 4th Edition, Zuckerman. A. J. et al., eds, John Wiley & Sons, NY, 1999; The Hepatitis C Viruses, by Hagedorn, C H et al., eds., Springer Verlag, 1999; Hepatitis B Virus: Molecular Mechanisms in Disease and Novel Strategies for Therapy, Koshy, R. et al., eds, World Scientific Pub Co, 1998; Veterinary Virology, Murphy, F. A. et al., eds., Academic Press, NY, 1999; Avian Viruses: Function and Control, Ritchie, B. W., Iowa State University Press, Ames, 2000; Virus Taxonomy: Classification and Nomenclature of Viruses: Seventh Report of the International Committee on Taxonomy of Viruses, by M. H. V. Van Regenmortel, M H V et al., eds., Academic Press; NY, 2000.

Plasmid DNA used for transfection or microinjection may be prepared using methods well-known in the art, for example using the Qiagen procedure (Qiagen), followed by DNA purification using known methods, such as the methods exemplified herein.

Such expression vectors may be used to transfect host cells (in vitro, ex vivo or in vivo) for expression of the DNA and production of the encoded proteins which include fusion proteins or peptides. In one embodiment, a nucleic acid (e.g., DNA) vaccine is administered to or contacted with a cell, e.g., a cell obtained from a subject (e.g., an antigen presenting cell), and administered to a subject, wherein the subject is treated before, after or at the same time as the cells are administered to the subject.

The term “isolated” as used herein, when referring to a molecule or composition, such as a translocation polypeptide or a nucleic acid coding therefor, means that the molecule or composition is separated from at least one other compound (protein, other nucleic acid, etc.) or from other contaminants with which it is natively associated or becomes associated during processing. An isolated composition can also be substantially pure. An isolated composition can be in a homogeneous state and can be dry or in aqueous solution. Purity and homogeneity can be determined, for example, using analytical chemical techniques such as polyacrylamide gel electrophoresis (PAGE) or high performance liquid chromatography (HPLC). Even where a protein has been isolated so as to appear as a homogenous or dominant band in a gel pattern, there are trace contaminants which co-purify with it.

Host cells transformed or transfected to express the fusion polypeptide or a homologue or functional derivative thereof are useful for the present invention. For example, the fusion polypeptide may be expressed in yeast, or mammalian cells such as Chinese hamster ovary cells (CHO) or human cells. In one embodiment, cells for expression according to the present invention are APCs or DCs. Other suitable host cells are known to those skilled in the art.

### Other Nucleic Acids for Potentiating Immune Responses

Methods of administrating a chemotherapeutic drug and a vaccine may further comprise administration of one or more other constructs, e.g., to prolong the life of antigen presenting cells. Exemplary constructs are described in the following two sections. Such constructs may be administered simultaneously or at the same time as a nucleic acid (e.g., DNA) vaccine. Alternatively, they may be administered before or after administration of the DNA vaccine or chemotherapeutic drug.

Potentiation of Immune Responses Using siRNA Directed at Apoptotic Pathways

Administration to a subject of a DNA vaccine and a chemotherapeutic drug may be accompanied by administration of one or more other agents, e.g., constructs. In one embodiment, a method comprises further administering to a subject an siRNA directed at an apoptotic pathway, such as described in WO 2006/073970, which is incorporated herein in its entirety.

The present inventors have designed siRNA sequences that hybridize to, and block expression of the activation of Bak and Bax proteins that are central players in the apoptosis signaling pathway. Methods of treating tumors or hyperproliferative diseases involving the administration of siRNA molecules (sequences), vectors containing or encoding the siRNA, expression vectors with a promoter operably linked to the siRNA coding sequence that drives transcription of siRNA sequences that are “specific” for sequences Bak and Bax nucleic acid are also encompassed within the present invention. siRNAs may include single stranded “hairpin” sequences because of their stability and binding to the target mRNA.

Since Bak and Bax are involved, among other death proteins, in apoptosis of APCs, particularly DCs, the present siRNA sequences may be used in conjunction with a broad range of DNA vaccine constructs encoding antigens to enhance and promote the immune response induced by such DNA vaccine constructs, particularly CD8+ T cell mediated immune responses typified by CTL activation and action. This is believed to occur as a result of the effect of the siRNA in prolonging the life of antigen-presenting DCs which may otherwise be killed in the course of a developing immune response by the very same CTLs that the DCs are responsible for inducing.

In addition to Bak and Bax, additional targets for siRNAs designed in an analogous manner include caspase 8, caspase 9 and caspase 3. The present invention includes compositions and methods in which siRNAs targeting any two or more of Bak, Bax, caspase 8, caspase 9 and caspase 3 are used in combination, optionally simultaneously (along with a DNA immunogen that encodes an antigen), to administer to a subject. Such combinations of siRNAs may also be used to transfect DCs (along with antigen loading) to improve the immunogenicity of the DCs as cellular vaccines by rendering them resistant to apoptosis.

siRNAs suppress gene expression through a highly regulated enzyme-mediated process called RNA interference (RNAi) (Sharp, P. A., Genes Dev. 15:485-90, 2001; Bernstein, E et al., Nature 409:363-66, 2001; Nykanen, A et al., Cell 107:309-21, 2001; Elbashir et al., Genes Dev. 15:188-200, 2001). RNA interference is the sequence-specific degradation of homologues in an mRNA of a targeting sequence in an siNA. As used herein, the term siNA (small, or short, interfering nucleic acid) is meant to be equivalent to other terms used to describe nucleic acid molecules that are capable of mediating sequence specific RNAi (RNA interference), for example short (or small) interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), translational silencing, and others. RNAi involves multiple RNA-protein interactions characterized by four major steps: assembly of siRNA with the RNA-induced silencing complex (RISC), activation of the RISC, target recognition and target cleavage. These interactions may bias strand selection during siRNA-RISC assembly and activation, and contribute to the overall efficiency of RNAi (Khvorova, A et al., Cell 115:209-216 (2003); Schwarz, D S et al. 115:199-208 (2003)))

Considerations to be taken into account when designing an RNAi molecule include, among others, the sequence to be targeted, secondary structure of the RNA target and binding of RNA binding proteins. Methods of optimizing siRNA sequences will be evident to the skilled worker. Typical algorithms and methods are described in Vickers et al. (2003) J Biol Chem 278:7108-7118; Yang et al. (2003) Proc Natl Acad Sci USA 99:9942-9947; Far et al. (2003) Nuc. Acids Res. 31:4417-4424; and Reynolds et al. (2004) Nature Biotechnology 22:326-330, all of which are incorporated by reference in their entirety.

The methods described in Far et al., supra, and Reynolds et al., supra, may be used by those of ordinary skill in the art to select targeted sequences and design siRNA sequences that are effective at silencing the transcription of the relevant mRNA. Far et al. suggests options for assessing target accessibility for siRNA and supports the design of active siRNA constructs. This approach can be automated, adapted to high throughput and is open to include additional parameters relevant to the biological activity of siRNA. To identify siRNA-specific features likely to contribute to efficient processing at each of the steps of RNAi noted above. Reynolds et al., supra, present a systematic analysis of 180 siRNAs targeting the mRNA of two genes. Eight characteristics associated with siRNA functionality were identified: low G/C content, a bias towards low internal stability at the sense strand 3′-terminus, lack of inverted repeats, and sense strand base preferences (positions 3, 10, 13 and 19). Application of an algorithm incorporating all eight criteria significantly improves potent siRNA selection. This highlights the utility of rational design for selecting potent siRNAs that facilitate functional gene knockdown.

Candidate siRNA sequences against mouse and human Bax and Bak are selected using a process that involves running a BLAST search against the sequence of Bax or Bak (or any other target) and selecting sequences that “survive” to ensure that these sequences will not be cross matched with any other genes.

siRNA sequences selected according to such a process and algorithm may be cloned into an expression plasmid and tested for their activity in abrogating Bak/Bax function cells of the appropriate animal species. Those sequences that show RNAi activity may be used by direct administration bound to particles, or recloned into a viral vector such as a replication-defective human adenovirus serotype 5 (Ad5).

One advantage of this viral vector is the high titer obtainable (in the range of 10¹⁰) and therefore the high multiplicities-of infection that can be attained. For example, infection with 100 infectious units/cell ensures all cells are infected. Another advantage of this virus is the high susceptibility and infectivity and the host range (with respect to cell types). Even if expression is transient, cells would survive, possibly replicate, and continue to function before Bak/Bax activity would recover and lead to cell death. In one embodiment, constructs include the following:

For Bak:

(SEQ ID NO: 42)

5′P-UGCCUACGAACUCUUCACCdTdT-3′
(sense)

(SEQ ID NO: 43)

5′P-GGUGAAGAGUUCGUAGGCAdTdT-3′
(antisense),

The nucleotide sequence encoding the Bak protein (including the stop codon) (GenBank accession No. NM_{—}007523 is shown herein as SEQ ID NO:44 with the targeted sequence in upper case, underscored. The targeted sequence of Bak, TGCCTACGAACTCTTCACC is shown herein as SEQ ID NO:45.

For Bax:

(SEQ ID NO: 46)

5′P-UAUGGAGCUGCAGAGGAUGdTdT-3′
(sense)

(SEQ ID NO: 47)

5′P-CAUCCUCUGCAGCUCCAUAdTdT-3′
(antisense)

The nucleotide sequence encoding Bax (including the stop codon) (GenBank accession No. L22472 is shown below (SEQ ID NO:48) with the targeted sequence shown in upper case and underscored

The targeted sequence of Bax, TATGGAGCTGCAGAGGATG is shown herein as SEQ ID NO:49

In a one embodiment, the inhibitory molecule is a double stranded nucleic acid (i.e., an RNA), used in a method of RNA interference. The following show the “paired” 19 nucleotide structures of the siRNA sequences shown above, where the symbol [symbol] :

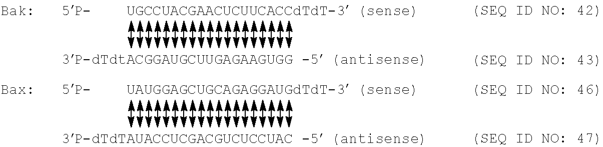

### Other Pro-Apoptotic Proteins to be Targeted

1. Caspase 8: The nucleotide sequence of human caspase-8 is shown herein as SEQ ID NO:50 (GenBank Access. # NM_{—}001228). One target sequence for RNAi is underscored. Others may be identified using methods such as those described herein (and in reference cited herein, primarily Far et al., supra and Reynolds et al., supra).

The sequences of sense and antisense siRNA strands for targeting this sequence including dTdT 3′ overhangs, are:

(SEQ ID NO: 51)

5′-AACCUCGGGGAUACUGUCUGAdTdT-3′ (sense)

(SEQ ID NO: 52)

5′-UCAGACAGUAUCCCCGAGGUUdTdT-3′ (antisense)

2. Caspase 9: The nucleotide sequence of human caspase-9 is shown herein as SEQ ID NO:53 (see GenBank Access. # NM_{—}001229). The sequence below is of “variant α” which is longer than a second alternatively spliced variant β, which lacks the underscored part of the sequence shown below (and which is anti-apoptotic). Target sequences for RNAi, expected to fall in the underscored segment, are identified using known methods such as those described herein and in Far et al., supra and Reynolds et al., supra) and siNAs, such as siRNAs, are designed accordingly.

3. Caspase 3: The nucleotide sequence of human caspase-3 is shown herein as SEQ ID NO: 54 (see GenBank Access. # NM_{—}004346). The sequence below is of “variant α” which is the longer of two alternatively spliced variants, all of which encode the full protein. Target sequences for RNAi are identified using known methods such as those described herein and in Far et al., supra and Reynolds et al., supra) and siNAs, such as siRNAs, are designed accordingly.

Long double stranded interfering RNAs, such a miRNAs, appear to tolerate mismatches more readily than do short double stranded RNAs. In addition, as used herein, the term RNAi is meant to be equivalent to other terms used to describe sequence specific RNA interference, such as post transcriptional gene silencing, or an epigenetic phenomenon. For example, siNA molecules useful for the invention can be used to epigenetically silence genes at both the post-transcriptional level or the pre-transcriptional level. In a non-limiting example, epigenetic regulation of gene expression by siNA molecules useful for the present invention can result from siNA mediated modification of chromatin structure and thereby alter gene expression (see, for example, Allshire Science 297:1818-19, 2002; Volpe et al., Science 297:1833-37, 2002; Jenuwein, Science 297:2215-18, 2002; and Hall et al., Science 297, 2232-2237, 2002.)

An siNA can be designed to target any region of the coding or non-coding sequence of an mRNA. An siNA is a double-stranded polynucleotide molecule comprising self-complementary sense and antisense regions, wherein the antisense region comprises nucleotide sequence that is complementary to nucleotide sequence in a target nucleic acid molecule or a portion thereof and the sense region has a nucleotide sequence corresponding to the target nucleic acid sequence or a portion thereof. The siNA can be assembled from two separate oligonucleotides, where one strand is the sense strand and the other is the antisense strand, wherein the antisense and sense strands are self-complementary. The siNA can be assembled from a single oligonucleotide, where the self-complementary sense and antisense regions of the siNA are linked by means of a nucleic acid based or non-nucleic acid-based linker(s). The siNA can be a polynucleotide with a hairpin secondary structure, having self-complementary sense and antisense regions. The siNA can be a circular single-stranded polynucleotide having two or more loop structures and a stem comprising self-complementary sense and antisense regions, wherein the circular polynucleotide can be processed either in vivo or in vitro to generate an active siNA molecule capable of mediating RNAi. The siNA can also comprise a single stranded polynucleotide having nucleotide sequence complementary to nucleotide sequence in a target nucleic acid molecule or a portion thereof (or can be an siNA molecule that does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al. (2002) Cell 110, 563-574 and Schwarz et al. (2002) Molecular Cell 10, 537-568), or 5′,3′-diphosphate.

In certain embodiments, the siNA molecule useful for the present invention comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, Van der Waal's interactions, hydrophobic interactions, and/or stacking interactions.

As used herein, siNA molecules need not be limited to those molecules containing only ribonucleotides but may also further encompass deoxyribonucleotides (as in the siRNAs which each include a dTdT dinucleotide) chemically-modified nucleotides, and non-nucleotides. In certain embodiments, the siNA molecules useful for the present invention lack 2′-hydroxy (2′-OH) containing nucleotides. In certain embodiments, siNAs do not require the presence of nucleotides having a 2′-hydroxy group for mediating RNAi and as such, siNAs useful for the present invention optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions. If modified, the siNAs useful for the present invention can also be referred to as “short interfering modified oligonucleotides” or “siMON.” Other chemical modifications, e.g., as described in Int'l Patent Publications WO 03/070918 and WO 03/074654, both of which are incorporated by reference, can be applied to any siNA sequence useful for the present invention.

In one embodiment a molecule mediating RNAi has a 2 nucleotide 3′ overhang (dTdT in the sequences disclosed herein). If the RNAi molecule is expressed in a cell from a construct, for example from a hairpin molecule or from an inverted repeat of the desired sequence, then the endogenous cellular machinery will create the overhangs.

Methods of making siRNAs are conventional. In vitro methods include processing the polyribonucleotide sequence in a cell-free system (e.g., digesting long dsRNAs with RNAse III or Dicer), transcribing recombinant double stranded DNA in vitro, and chemical synthesis of nucleotide sequences homologous to Bak or Bax sequences. See, e.g., Tuschl et al., Genes & Dev. 13:3191-3197, 1999. In vivo methods include
(1) transfecting DNA vectors into a cell such that a substrate is converted into siRNA in vivo. See, for example, Kawasaki et al., Nucleic Acids Res 31:700-07, 2003; Miyagishi et al., Nature Biotechnol 20:497-500, 2003; Lee et al., Nature Biotechnol 20:500-05, 2002; Brummelkamp et al., Science 296:550-53, 2002; McManus et al., RNA 8:842-50, 2002; Paddison et al., Genes Dev 16:948-58, 2002; Paddison et al., Proc Natl Acad Sci USA 99:1443-48, 2002; Paul et al., Nature Biotechnol 20:505-08, 2002; Sui et al., Proc Natl Acad Sci USA 99:5515-20, 2002; Yu et al., Proc Natl Acad Sci USA 99:6047-52, 2002)
(2) expressing short hairpin RNAs from plasmid systems using RNA polymerase III (pol III) promoters. See, for example, Kawasaki et al., supra; Miyagishi et al., supra; Lee et al., supra; Brummelkamp et al., supra; McManus et al., supra), Paddison et al., supra (both); Paul et al., supra, Sui et al., supra; and Yu et al., supra; and/or
(3) expressing short RNA from tandem promoters. See, for example, Miyagishi et al., supra; Lee et al., supra).

When synthesized in vitro, a typical micromolar scale RNA synthesis provides about 1 mg of siRNA, which is sufficient for about 1000 transfection experiments using a 24-well tissue culture plate format. In general, to inhibit Bak or Bax expression in cells in culture, one or more siRNAs can be added to cells in culture media, typically at about 1 ng/ml to about 10 μg siRNA/ml.

For reviews and more general description of inhibitory RNAs, see Lau et al., Sci Amer August 2003: 34-41; McManus et al., Nature Rev Genetics 3, 737-47, 2002; and Dykxhoorn et al., Nature Rev Mol Cell Bio 4:457-467, 2003. For further guidance regarding methods of designing and preparing siRNAs, testing them for efficacy, and using them in methods of RNA interference (both in vitro and in vivo), see, e.g., Allshire, Science 297:1818-19, 2002; Volpe et al., Science 297:1833-37, 2002; Jenuwein, Science 297:2215-18, 2002; Hall et al., Science 297 2232-37, 2002; Hutvagner et al., Science 297:2056-60, 2002; McManus et al. RNA 8:842-850, 2002; Reinhart et al., Genes Dev. 16:1616-26, 2002; Reinhart et al., Science 297:1831, 2002; Fire et al. (1998) Nature 391:806-11, 2002; Moss, Curr Biol 11:R772-5, 2002:Brummelkamp et al., supra; Bass, Nature 411 428-9, 2001; Elbashir et al., Nature 411:494-8; U.S. Pat. No. 6,506,559; Published US Pat App. 20030206887; and PCT applications WO99/07409, WO99/32619, WO 00/01846, WO 00/44914, WO00/44895, WO01/29058, WO01/36646, WO01/75164, WO01/92513, WO 01/29058, WO01/89304, WO01/90401, WO02/16620, and WO02/29858, all of which are incorporated by reference.

Ribozymes and siNAs can take any of the forms, including modified versions, described for antisense nucleic acid molecules; and they can be introduced into cells as oligonucleotides (single or double stranded), or in the form of an expression vector.

In one embodiment, an antisense nucleic acid, siNA (e.g., siRNA) or ribozyme comprises a single stranded polynucleotide comprising a sequence that is at least about 90% (e.g., at least about 93%, 95%, 97%, 98% or 99%) identical to a target segment (such as those indicted for Bak and Bax above) or a complement thereof. As used herein, a DNA and an RNA encoded by it are said to contain the same “sequence,” taking into account that the thymine bases in DNA are replaced by uracil bases in RNA.

Active variants (e.g., length variants, including fragments; and sequence variants) of the nucleic acid-based inhibitors discussed herein are also within the scope of the present invention. An “active” variant is one that retains an activity of the inhibitor from which it is derived (i.e., the ability to inhibit expression). It is to test a variant to determine for its activity using conventional procedures.

As for length variants, an antisense nucleic acid or siRNA may be of any length that is effective for inhibition of a gene of interest. Typically, an antisense nucleic acid is between about 6 and about 50 nucleotides (e.g., at least about 12, 15, 20, 25, 30, 35, 40, 45 or 50 nt), and may be as long as about 100 to about 200 nucleotides or more. Antisense nucleic acids having about the same length as the gene or coding sequence to be inhibited may be used. When referring to length, the terms bases and base pairs (bp) are used interchangeably, and will be understood to correspond to single stranded (ss) and double stranded (ds) nucleic acids. The length of an effective siNA is generally between about 15 bp and about 29 bp in length, between about 19 and about 29 bp (e.g., about 15, 17, 19, 21, 23, 25, 27 or 29 bp), with shorter and longer sequences being acceptable. Generally, siNAs are shorter than about 30 bases to prevent eliciting interferon effects. For example, an active variant of an siRNA having, for one of its strands, the 19 nucleotide sequence of any of SEQ ID NOs:42, 43, 46, and 47 herein can lack base pairs from either, or both, of ends of the dsRNA; or can comprise additional base pairs at either, or both, ends of the ds RNA, provided that the total of length of the siRNA is between about 19 and about 29 bp, inclusive. One embodiment useful for the present invention is an siRNA that “consists essentially of” sequences represented by SEQ ID NOs:42, 43, 46, and 47 or complements of these sequence. An siRNA useful for the present invention may consist essentially of between about 19 and about 29 bp in length.

As for sequence variants, in one embodiment, an inhibitory nucleic acid, whether an antisense molecule, a ribozyme (the recognition sequences), or an siNA, comprises a strand that is complementary (100% identical in sequence) to a sequence of a gene that it is designed to inhibit. However, 100% sequence identity is not required to practice the present invention. Thus, the invention has the advantage of being able to tolerate naturally occurring sequence variations, for example, in human c-met, that might be expected due to genetic mutation, polymorphism, or evolutionary divergence. Alternatively, the variant sequences may be artificially generated. Nucleic acid sequences with small insertions, deletions, or single point mutations relative to the target sequence can be effective inhibitors.

The degree of sequence identity may be optimized by sequence comparison and alignment algorithms well-known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). In one embodiment, at least about 90% sequence identity may be used (e.g., at least about 92%, 95%, 98% or 99%), or even 100% sequence identity, between the inhibitory nucleic acid and the targeted sequence of targeted gene.

Alternatively, an active variant of an inhibitory nucleic acid useful for the present invention is one that hybridizes to the sequence it is intended to inhibit under conditions of high stringency. For example, the duplex region of an siRNA may be defined functionally as a nucleotide sequence that is capable of hybridizing with a portion of the target gene transcript under high stringency conditions (e.g., 400 mM NaCl, 40 mM PIPES pH 6.4, 1 mM EDTA, 50° C. or 70° C., hybridization for 12-16 hours), followed generally by washing.

DC-1 cells or BM-DCs presenting a given antigen X, when not treated with the siRNAs useful for the present invention, respond to sufficient numbers X-specific CD8+ CTL by apoptotic cell death. In contrast, the same cells transfected with the siRNA or infected with a viral vector encoding the present siRNA sequences survive better despite the delivery of killing signals.

Delivery and expression of the siRNA compositions useful for the present invention inhibit the death of DCs in vivo in the process of a developing T cell response, and thereby promote and stimulate the generation of an immune response induced by immunization with an antigen-encoding DNA vaccine vector. These capabilities have been exemplified by showing that:
(1) co-administration of DNA vaccines encoding HPV-16 E7 with siRNA targeted to Bak and Bax prolongs the lives of antigen-presenting DCs in the draining lymph nodes, thereby enhancing antigen-specific CD8⁺ T cell responses, and eliciting potent antitumor effects against an E7-expressing tumor in vaccinated subjects.
(2) DCs transfected with siRNA targeting Bak and Bax resist killing by T cells in vivo. E7-loaded DCs transfected with Bak/Bax siRNA so that Bak and Bax protein expression is downregulated resist apoptotic death induced by T cells in vivo. When administered to subjects, these DCs generate stronger antigen-specific immune responses and manifest therapeutic effects (compared to DCs transfected with control siRNA).

Thus, siRNA constructs are useful as a part of the nucleic acid vaccination and chemotherapy regimen described in this application.

### Potentiation of Immune Responses Using Anti-Apoptotic Proteins

Administration to a subject of a DNA vaccine and a chemotherapeutic drug may also be accompanied by administration of a nucleic acid encoding an anti-apoptotic protein, as described in WO2005/047501 and in U.S. Patent Application Publication No. 20070026076, both of which are incorporated by reference.

The present inventors have designed and disclosed an immunotherapeutic strategy that combines antigen-encoding DNA vaccine compositions with additional DNA vectors comprising anti-apoptotic genes including bc1-2, bc-1xL, XIAP, dominant negative mutants of caspase-8 and caspase-9, the products of which are known to inhibit apoptosis (Wu, et al. U.S. Patent Application Publication No. 20070026076, incorporated herein by reference). Serine protease inhibitor 6 (SPI-6) which inhibits granzyme B, may also be employed in compositions and methods to delay apoptotic cell death of DCs. The present inventors have shown that the harnessing of an additional biological mechanism, that of inhibiting apoptosis, significantly enhances T cell responses to DNA vaccines comprising antigen-coding sequences, as well as linked sequences encoding such IPPs.

Intradermal vaccination by gene gun efficiently delivers a DNA vaccine into DCs of the skin, resulting in the activation and priming of antigen-specific T cells in vivo. DCs, however, have a limited life span, hindering their long-term ability to prime antigen-specific T cells. According to the present invention, a strategy that combines combination therapy with methods to prolong the survival of DNA-transduced DCs enhances priming of antigen-specific T cells and thereby, increase DNA vaccine potency. Co-delivery of DNA encoding inhibitors of apoptosis (BCL-xL, BCL-2, XIAP, dominant negative caspase-9, or dominant negative caspase-8) with DNA encoding an antigen (exemplified as HPV-16 E7 protein) prolongs the survival of transduced DCs. More importantly, vaccinated subjects exhibited significant enhancement in antigen-specific CD8+ T cell immune responses, resulting in a potent antitumor effect against antigen-expressing tumors. Among these anti-apoptotic factors, BCL-XL demonstrated the greatest enhancement of both antigen-specific immune responses and antitumor effects. Thus, co-administration of a combination therapy including a DNA vaccine with one or more DNA constructs encoding anti-apoptotic proteins provides a way to enhance DNA vaccine potency.

Serine protease inhibitor 6 (SPI-6), also called Serpinb9, inhibits granzyme B, and may thereby delay apoptotic cell death in DCs. Intradermal co-administration of DNA encoding SPI-6 with DNA constructs encoding E7 linked to various IPPs significantly increased E7-specific CD8+ T cell and CD4+ Th1 cell responses and enhanced anti-tumor effects when compared to vaccination without SPI-6. Thus, in certain embodiments, combined methods are used that enhance MHC class I and II antigen processing with delivery of SPI-6 to potentiate immunity.

A similar approach employs DNA-based alphaviral RNA replicon vectors, also called suicidal DNA vectors. To enhance the immune response to an antigen, e.g., HPV E7, a DNA-based Semliki Forest virus vector, pSCA1, the antigen DNA is fused with DNA encoding an anti-apoptotic polypeptide such BCL-xL, a member of the BCL-2 family. pSCA1 encoding a fusion protein of an antigen polypeptide and/BCL-xL delays cell death in transfected DCs and generates significantly higher antigen-specific CD8+ T-cell-mediated immunity. The antiapoptotic function of BCL-xL is important for the enhancement of antigen-specific CD8+ T-cell responses. Thus, in one embodiment, delaying cell death induced by an otherwise desirable suicidal DNA vaccine enhances its potency.

Thus, the present invention is also directed to combination therapies including administering a chemotherapeutic drug with a nucleic acid composition useful as an immunogen, comprising a combination of: (a) first nucleic acid vector comprising a first sequence encoding an antigenic polypeptide or peptide, which first vector optionally comprises a second sequence linked to the first sequence, which second sequence encodes an immunogenicity-potentiating polypeptide (IPP); b) a second nucleic acid vector encoding an anti-apoptotic polypeptide, wherein, when the second vector is administered with the first vector to a subject, a T cell-mediated immune response to the antigenic polypeptide or peptide is induced that is greater in magnitude and/or duration than an immune response induced by administration of the first vector alone. The first vector above may comprise a promoter operatively linked to the first and/or the second sequence.

In the above compositions the anti-apoptotic polypeptide may be selected from the group consisting of (a) BCL-xL, (b) BCL2, (c) XIAP, (d) FLICEc-s, (e) dominant-negative caspase-8, (f) dominant negative caspase-9, (g) SPI-6, and (h) a functional homologue or a derivative of any of (a)-(g). The anti-apoptotic DNA may be physically linked to the antigen-encoding DNA. Examples of this are provided in U.S. Patent Application publication No. 20070026076, incorporated by reference, primarily in the form of suicidal DNA vaccine vectors. Alternatively, the anti-apoptotic DNA may be administered separately from, but in combination with the antigen-encoding DNA molecule. Even more examples of the co-administration of these two types of vectors are provided in U.S. patent application Ser. No. 10/546,810 (publication number US 2007-0026076).

Exemplary nucleotide and amino acid sequences of anti-apoptotic and other proteins are provided in the sequence listing. Biologically active homologs of these proteins and constructs may also be used. Biologically active homologs is to be understood as described herein in the context of other proteins, e.g., IPPs.

The coding sequence for BCL-xL as present in the pcDNA3 vector useful for the present invention is SEQ ID NO:55; the amino acid sequence of BCL-xL is SEQ ID NO:56; the sequence pcDNA3-BCL-xL is SEQ ID NO:57 (the BCL-xL coding sequence corresponds to nucleotides 983 to 1732); a pcDNA3 vector combining E7 and BCL-xL, designated pcDNA3-E7/BCL-xL is SEQ ID NO:58 (the E7 and BCL-xL sequences correspond to nucleotides 960 to 2009); the amino acid sequence of the E7-BCL-xL chimeric or fusion polypeptide is SEQ ID NO:59; a mutant BCL-xL (“mtBCL-xL”) DNA sequence is SEQ ID NO:60; the amino acid sequence of mtBCL-xL is SEQ ID NO:61; the amino acid sequence of the E7-mtBCL-xL chimeric or fusion polypeptide is SEQ ID NO:62; in the pcDNA-mtBCL-xL [SEQ ID NO:63] vector, this mutant sequence is inserted in the same position that BCL-xL is inserted in SEQ ID NO:57 and in the pcDNA-E7/mtBCL-XL [SEQ ID NO:64], this sequence is inserted in the same position as the BCL-xL sequence is in SEQ ID NO:58; the sequence of the suicidal DNA vector pSCA1-BCL-xL is SEQ ID NO:65 (the BCL-xL sequence corresponds to nucleotides 7483 to 8232); the sequence of the “combined” vector, pSCA1-E7/BCL-xL is SEQ ID NO:66 (the sequence of E7 and BCL-xL corresponds to nucleotides 7461 to 8510); the sequence of pSCA1-mtBCL-xL [SEQ ID NO:67] is the same as that for the wild type BCL-xL except that the mtBCL-xL sequence is inserted in the same position as the wild type sequence in the pSCA1-mtBCL-xL vector; the sequence pSCA1-E7/mtBCL-xL [SEQ ID NO:68] is the same as that for the wild type pSCA1-E7/BCL-xL above, except that the mtBCL-xL sequence is inserted in the same position as the wild type sequence; the sequence of the vector pSGS-BCL-xL is SEQ ID NO:69 (the BCL-xL coding sequence corresponds to nucleotides 1061 to 1810); the sequenced of the vector pSGS-mtBCL-xL is SEQ ID NO:70 with the mutant BCL-xL sequence has the mtBCL-xL, shown above, inserted in the same location as for the wild type vector immediately above; the nucleotide sequence of the DNA encoding the XIAP anti-apoptotic protein is SEQ ID NO:71; the amino acid of the vector comprising the XIAP anti-apoptotic protein coding sequence is SEQ ID NO:72; the nucleotide sequence of the vector comprising the XIAP anti-apoptotic protein coding sequence, designated PSGS-XIAP is shown in SEQ ID NO:73 (with the XIAP corresponding to nucleotides 1055 to 2553); the sequence of DNA encoding the anti-apoptotic protein FLICEc-s is SEQ ID NO:74; the amino acid sequence of the anti-apoptotic protein FLICEc-s is SEQ ID NO:75; the PSGS vector encoding the anti-apoptotic protein FLICEc-s, designated PSGS-FLICEc-s, has the sequence SEQ ID NO:76 (with the FLICEc-s sequence corresponding to nucleotides 1049 to 2443); the sequence of DNA encoding the anti-apoptotic protein Bc12 is SEQ ID NO:77; the amino acid sequence of Bc12 is SEQ ID NO:78; the PSGS vector encoding Bc12, designated PSGS-BCL2, has the sequence SEQ ID NO:79 (with the Bc12 sequence corresponding to nucleotides 1061 to 1678); the pSGS-dn-caspase-8 vector is SEQ ID NO:80 (encoding the dominant-negative caspase-8 corresponding to nucleotides 1055 to 2449); the amino acid sequence of dn-caspase-8 is SEQ ID NO:81; the pSGS-dn-caspase-9 vector is SEQ ID NO:82 (encoding the dominant-negative caspase-9 as nucleotides 1055 to 2305); the amino acid sequence of dn-caspase-9 is SEQ ID NO:83; the nucleotide sequence of murine serine protease inhibitor 6 (SPI-6, deposited in GENEBANK as NM 009256) is SEQ ID NO:84; the amino acid sequence of the SPI-6 protein is SEQ ID NO:85; the nucleic acid sequence of the mutant SPI-6 (mtSPI6) is SEQ ID NO:86; the amino acid sequence of the mutant SPI-6 protein (mtSPI-6) is SEQ ID NO:87; the sequence of the pcDNA3-Spi6 vector is SEQ ID NO:88 (the SPI-6 sequence corresponds to nucleotides 960 to 2081); and the sequence of the mutant vector pcDNA3-mtSpi6 vector [SEQ ID NO:89] is the same as that above, except that the mtSPI-6 sequence is inserted in the same location in place of the wild type SPI-6.

Biologically active homologs of these nucleic acids and proteins may be used. Biologically active homologs are to be understood as described in the context of other proteins, e.g., IPPs, herein. For example, a vector may encode an anti-apoptotic protein that is at least about 90%, 95%, 98% or 99% identical to that of a sequence set forth herein.

### MHC Class I/II Activators

“MHC class I/II activators” refers to molecules or complexes thereof that increase immune responses by increasing MHC class I or II (“I/II”) antigen presentation, such as by increasing MHC class I, class II or class I and class II activity or gene expression. In one embodiment, an MHC class I/II activator is a nucleic acid encoding a protein that enhances MHC class I/II antigen presentation. Exemplary MHC class I/II activators include nucleic acids encoding an MHC class II associated invariant chain (Ii), in which the CLIP region is replaced with a T cell epitope, e.g., a promiscuous T cell epitope, such as the Pan HLA-DR reactive epitope (PADRE), or a variant thereof. Other MHC class I/II activators are nucleic acids encoding the MHC class II transactivator CIITA or a variant thereof.

In one embodiment, an MHC class I/II activator is a nucleic acid, e.g., an isolated nucleic acid, encoding a protein comprising, consisting or consisting essentially of an invariant (Ii) chain, wherein the CLIP region is replaced with a promiscuous CD4+ T cell epitope. A “promiscuous CD4+ T cell epitope” is used interchangeably with “universal CD4+ T cell epitope” and refers to peptides that bind to numerous histocompatibility alleles, e.g., human MHC class II molecules. In one embodiment, the promiscuous CD4+ T cell epitope is a Pan HLA-DR reactive epitope (PADRE), thereby forming an Ii-PADRE protein that is encoded by an Ii-PADRE nucleic acid. In one embodiment, a nucleic acid encodes an Ii chain, wherein amino acids 81-102 (KPVSQMRMATPLLMRPM (SEQ ID NO:92) are replaced with the PADRE sequence AKFVAAWTLKAAA (SEQ ID NO:93). An exemplary human Ii-PADRE amino acid sequence is set forth as SEQ ID NO:91, and is encoded by nucleotide sequence SEQ ID NO:90.

Also provided herein are variants of a protein consisting of SEQ ID NO:91. A protein may comprise, consist essentially of, or consist of an amino acid sequence that is at least about 80%, 85%, 90%, 95%, 96%, 97%, 98% or 99% identical to SEQ ID NO:91. A protein may comprise a PADRE that is identical to the PADRE of SEQ ID NO:91, i.e., consisting of SEQ ID NO:93. A protein may comprise a PADRE sequence that is at least about 80%, 85%, 90%, 95%, 96%, 97%, 98% or 99% identical to SEQ ID NO:93; and/or an Ii sequence that is at least about 80%, 85%, 90%, 95%, 96%, 97%, 98% or 99% identical to the Ii sequence of SEQ ID NO:91.

An amino acid sequence may differ from that of SEQ ID NO:91 or the Ii or PADRE sequences thereof by the addition, deletion or substitution of at least about 1, 2, 3, 4, 5, 6, 7, 8, 9, 10, 11, 12, 13, 14, 15, 16, 17, 18, 19, 20, 25, 30 or more amino acids. In certain embodiments, a protein lacks one or more, e.g., 2, 3, 4, 5, 6, 7, 8, 9, 10 or more amino acids at the C- and/or N-terminus and/or internal relative to that of SEQ ID NO:91 or the Ii or PADRE region thereof. In certain embodiments, an amino acid sequence differs from that of SEQ ID NO:93 or from that of the Ii sequence by the addition, deletion or substitution of at least about 1, 2, 3, 4, or 5 amino acids.

Variants of SEQ ID NO:91 or the PADRE or Ii regions thereof preferably have a biological activity. Such variants are referred to as “functional homologs” or “functional variants.” Functional homologs include variants of SEQ ID NO:91 that increase an immune response, e.g., an antigen specific immune response, in a subject to whom it is administered, or has any of the biological activities set forth in the Examples pertaining to Ii-PADRE. Variants of the PADRE sequence or the Ii sequence may have a biological activity that is associated with that of the wild type PADRE or Ii sequences, respectively. Biological activities can be determined as know in the art or as set forth in the Examples. In addition, comparison (or alignment) of the Ii and PADRE sequences from different species is expected to be helpful in determining which amino acids may be varied and which ones should preferably not be varied.

Other proteins provided herein comprise a PADRE amino acid sequence that replaces a larger portion of Ii, e.g., wherein Ii is lacking about amino acids 81-103, 81-104, 81-105, 81-106, 81-107, 81-108, 81-109, 81-110 or more; is lacking about amino acids 70-102, 71-102, 72-102, 73-102, 74-102, 75-102, 76-102, 77-102, 78-102, 79-102, 80-102 or more.

Other promiscuous CD4+ T cell epitopes that may be used instead of PADRE are listed in Table 1.

TABLE 1
Exemplary promiscuous CD4+ T cell epitopes

| | |
|---|---|
| Promiscuous CD4+ T cell epitopes | Reference |

| | |
|---|---|
| EBV-latent membrane protein 1(LMP1₁₅₉₋₁₇₅) | (1) |
| YLQQNWWTLLVDLLWLL (SEQ ID NO: 119) | |
| MAGE-A6₁₇₂₋₁₈₇; IGHVYIFATCLGLSYD (SEQ ID NO: 120) | (2) |
| Mycoplasma penetrans HF-2₂₁₉₋₂₂₆; IYIFAACL (SEQ ID NO: 121) | |
| six-transmembrane epithelial antigen of prostate (STEAP) | (3) |
| STEAP₁₀₂₋₁₁₆HQQYFYKIPILVINK (SEQ ID NO: 122) | |
| STEAP₁₉₂₋₂₀₆LLNWAYQQVQQNKED (SEQ ID NO: 123) | |
| Taxol-resistance-associated gene-3 (TRAG3)₃₅₋₄₈ | (4) |
| EFHACW PAFTVLGE (SEQ ID NO: 124) | |
| Survivin₁₀₋₂₄ WQPFLKDHRISTFKN (SEQ ID NO: 125) | (5) |
| HPV 18-E6₅₂₋₆₆; LFVVYRDSIPHAACH (SEQ ID NO: 126) | (6) |
| HPV18-E6₉₇₋₁₁₁; GLYNLLIRCLRCQKP (SEQ ID NO: 127) | |
| Carcinoembryonic antigen₁₇₇₋₁₈₉; LWWVNNQSLPVSP (SEQ ID | (7) |
| NO: 128) | |
| mycobacterial antigen MPB70 | (8) |
| MPB70₁₀₆₋₁₃₀; FSKLPASTIDELKTNSSLLTSILTY (SEQ ID NO: | |
| 129) | |
| MPB70₁₆₆₋₁₉₃; GNADVVCGGVSTANATVYMIDSVLMPPA (SEQ | |
| ID NO: 130) | |
| HER-2₇₇₆₋₇₈₈ GSPYVSRLLGICL (SEQ ID NO: 131) | (9) |
| HER-2₈₃₃₋₈₄₉KVPIKWMALESILRRRF (SEQ ID NO: 132) | (10) |
| NY-ESO-1₁₁₉₋₁₄₃ PGVLLKEFTVSGNILTIRLTAADHR (SEQ ID | (11) |
| NO: 133) | |
| Tetanus toxin₁₀₈₄₋₁₀₉₉ VSIDKFRIFCKANPK (SEQ ID NO: 134) | (12) |
| Tetanus toxin₁₁₇₄₋₁₁₈₉ LKFIIKRYTPNNEIDS (SEQ ID NO: 135) | |
| Tetanus toxin₁₀₆₄₋₁₀₇₉ IREDNNITLKLDRCN (SEQ ID NO: 136) | |
| Tetanus toxin₉₄₇₋₉₆₇ FNNFTVSFWLRVPKVSASHLE (SEQ ID NO: | |
| 137) | |
| Tetanus toxin₈₃₀₋₈₄₃ QYIKANSKFIGITE (SEQ ID NO: 138) | |
| HBV nuclear capside₅₀₋₆₉ PHHTALRQAILCWGELMTLA (SEQ ID | |
| NO: 139) | |
| Influenza haemagglutinin₃₀₇₋₃₁₉ PKYVKQNTLKLAT (SEQ ID NO: | |
| 140) | |
| HBV surface antigen₁₉₋₃₃-FFLLTRILTIPQSLD (SEQ ID NO: 141) | |
| Influenza matrix₁₇₋₃₁ YSGPLKAEIAQRLEDV (SEQ ID NO: 142) | |
| P. falciparum CSP₃₈₀₋₃₉₈ EKKIAKMEKASSVFNVVN (SEQ ID NO: | |
| 143) | |

1. Kobayashi, H., T. Nagato, M. Takahara, K. Sato, S. Kimura, N. Aoki, M. Azumi, M. Tateno, Y. Harabuchi, and E. Celis. 2008. Induction of EBV-latent membrane protein 1-specific MHC class II-restricted T-cell responses against natural killer lymphoma cells. Cancer Res 68:901-908.
2. Vujanovic, L., M. Mandic, W. C. Olson, J. M. Kirkwood, and W. J. Storkus. 2007. A mycoplasma peptide elicits heteroclitic CD4+ T cell responses against tumor antigen MAGE-A6. Clin Cancer Res 13:6796-6806.
3. Kobayashi, H., T. Nagato, K. Sato, N. Aoki, S. Kimura, M. Murakami, H. Iizuka, M. Azumi, H. Kakizaki, M. Tateno, and E. Celis. 2007. Recognition of prostate and melanoma tumor cells by six-transmembrane epithelial antigen of prostate-specific helper T lymphocytes in a human leukocyte antigen class II-restricted manner. Cancer Res 67:5498-5504.
4. Janjic, B., P. Andrade, X. F. Wang, J. Fourcade, C. Almunia, P. Kudela, A. Brufsky, S. Jacobs, D. Friedland, R. Stoller, D. Gillet, R. B. Herberman, J. M. Kirkwood, B. Maillere, and H. M. Zarour. 2006. Spontaneous CD4+ T cell responses against TRAG-3 in patients with melanoma and breast cancers. J Immunol 177:2717-2727.
5. Piesche, M., Y. Hildebrandt, F. Zettl, B. Chapuy, M. Schmitz, G. Wulf, L. Trumper, and R. Schroers. 2007. Identification of a promiscuous HLA DR-restricted T-cell epitope derived from the inhibitor of apoptosis protein survivin. Hum Immunol 68:572-576.
6. Facchinetti, V., S. Seresini, R. Longhi, C. Garavaglia, G. Casorati, and M. P. Protti. 2005. CD4+ T cell immunity against the human papillomavirus-18 E6 transforming protein in healthy donors: identification of promiscuous naturally processed epitopes. Eur J Immunol 35:806-815.
7. Campi, G., M. Crosti, G. Consogno, V. Facchinetti, B. M. Conti-Fine, R. Longhi, G. Casorati, P. Dellabona, and M. P. Protti. 2003. CD4(+) T cells from healthy subjects and colon cancer patients recognize a carcinoembryonic antigen-specific immunodominant epitope. Cancer Res 63:8481-8486.
8. Al-Attiyah, R., F. A. Shaban, H. G. Wiker, F. Oftung, and A. S. Mustafa. 2003. Synthetic peptides identify promiscuous human Th1 cell epitopes of the secreted mycobacterial antigen MPB70. Infect Immun 71:1953-1960.
9. Sotiriadou, R., S. A. Perez, A. D. Gritzapis, P. A. Sotiropoulou, H. Echner, S. Heinzel, A. Mamalaki, G. Pawelec, W. Voelter, C. N. Baxevanis, and M. Papamichail. 2001. Peptide HER2(776-788) represents a naturally processed broad MHC class II-restricted T cell epitope. Br J Cancer 85:1527-1534.
10. Kobayashi, H., M. Wood, Y. Song, E. Appella, and E. Celis. 2000. Defining promiscuous MHC class II helper T-cell epitopes for the HER2/neu tumor antigen. Cancer Res 60:5228-5236.
11. Zarour, H. M., B. Maillere, V. Brusic, K. Coval, E. Williams, S. Pouvelle-Moratille, F. Castelli, S. Land, J. Bennouna, T. Logan, and J. M. Kirkwood. 2002. NY-ESO-1 119-143 is a promiscuous major histocompatibility complex class II T-helper epitope recognized by Th1- and Th2-type tumor-reactive CD4+ T cells. Cancer Res 62:213-218.
12. Falugi, F., R. Petracca, M. Mariani, E. Luzzi, S. Mancianti, V. Carinci, M. L. Melli, O. Finco, A. Wack, A. Di Tommaso, M. T. De Magistris, P. Costantino, G. Del Giudice, S. Abrignani, R. Rappuoli, and G. Grandi. 2001. Rationally designed strings of promiscuous CD4(+) T cell epitopes provide help to Haemophilus influenzae type b oligosaccharide: a model for new conjugate vaccines. Eur J Immunol 31:3816-3824.

The CLIP region in an Ii molecule, e.g., having the amino acid sequence of the Ii portion set forth in SEQ ID NO:91, may be replaced with any of the peptides in Table 2 or other promiscuous epitopes set forth in the references of Table 2, or functional variants thereof. Preferred epitopes include those from tetanus toxin and influenza. Any other promiscuous CD4+ T cell epitopes may be used, e.g., those described in the following references:
1. Campi, G., M. Crosti, G. Consogno, V. Facchinetti, B. M. Conti-Fine, R. Longhi, G. Casorati, P. Dellabona, and M. P. Protti. 2003. CD4(+) T cells from healthy subjects and colon cancer patients recognize a carcinoembryonic antigen-specific immunodominant epitope. Cancer Res 63:8481-8486.
2. Castelli, F. A., M. Leleu, S. Pouvelle-Moratille, S. Farci, H. M. Zarour, M. Andrieu, C. Auriault, A. Menez, B. Georges, and B. Maillere. 2007. Differential capacity of T cell priming in naive donors of promiscuous CD4+ T cell epitopes of HCV NS3 and Core proteins. Eur J Immunol 37:1513-1523.
3. Consogno, G., S. Manici, V. Facchinetti, A. Bachi, J. Hammer, B. M. Conti-Fine, C. Rugarli, C. Traversari, and M. P. Protti. 2003. Identification of immunodominant regions among promiscuous HLA-DR-restricted CD4+ T-cell epitopes on the tumor antigen MAGE-3. Blood 101:1038-1044.
4. Depil, S., O. Morales, F. A. Castelli, N. Delhem, V. Francois, B. Georges, F. Dufosse, F. Morschhauser, J. Hammer, B. Maillere, C. Auriault, and V. Pancre. 2007. Determination of a HLA II promiscuous peptide cocktail as potential vaccine against EBV latency II malignancies. J Immunother (1997) 30:215-226.
5. Facchinetti, V., S. Seresini, R. Longhi, C. Garavaglia, G. Casorati, and M. P. Protti. 2005. CD4+ T cell immunity against the human papillomavirus-18 E6 transforming protein in healthy donors: identification of promiscuous naturally processed epitopes. Eur J Immunol 35:806-815.
6. Kobayashi, H., T. Nagato, K. Sato, N. Aoki, S. Kimura, M. Murakami, H. Iizuka, M. Azumi, H. Kakizaki, M. Tateno, and E. Celis. 2007. Recognition of prostate and melanoma tumor cells by six-transmembrane epithelial antigen of prostate-specific helper T lymphocytes in a human leukocyte antigen class II-restricted manner. Cancer Res 67:5498-5504.
7. Kobayashi, H., M. Wood, Y. Song, E. Appella, and E. Celis. 2000. Defining promiscuous MHC class II helper T-cell epitopes for the HER2/neu tumor antigen. Cancer Res 60:5228-5236.
8. Mandic, M., C. Almunia, S. Vicel, D. Gillet, B. Janjic, K. Coval, B. Maillere, J. M. Kirkwood, and H. M. Zarour. 2003. The alternative open reading frame of LAGE-1 gives rise to multiple promiscuous HLA-DR-restricted epitopes recognized by T-helper 1-type tumor-reactive CD4+ T cells. Cancer Res 63:6506-6515.
9. Neumann, F., C. Wagner, S. Stevanovic, B. Kubuschok, C. Schormann, A. Mischo, K. Ertan, W. Schmidt, and M. Pfreundschuh. 2004. Identification of an HLA-DR-restricted peptide epitope with a promiscuous binding pattern derived from the cancer testis antigen HOM-MEL-40/SSX2. Int J Cancer 112:661-668.
10. Ohkuri, T., M. Sato, H. Abe, K. Tsuji, Y. Yamagishi, H. Ikeda, N. Matsubara, H. Kitamura, and T. Nishimura. 2007. Identification of a novel NY-E50-1 promiscuous helper epitope presented by multiple MHC class II molecules found frequently in the Japanese population. Cancer Sci 98:1092-1098.
11. Piesche, M., Y. Hildebrandt, F. Zettl, B. Chapuy, M. Schmitz, G. Wulf, L. Trumper, and R. Schroers. 2007. Identification of a promiscuous HLA DR-restricted T-cell epitope derived from the inhibitor of apoptosis protein survivin. Hum Immunol 68:572-576.
12. Sotiriadou, R., S. A. Perez, A. D. Gritzapis, P. A. Sotiropoulou, H. Echner, S. Heinzel, A. Mamalaki, G. Pawelec, W. Voelter, C. N. Baxevanis, and M. Papamichail. 2001. Peptide HER2(776-788) represents a naturally processed broad MHC class II-restricted T cell epitope. Br J Cancer 85:1527-1534.
13. Texier, C., S. Pouvelle-Moratille, C. Buhot, F. A. Castelli, C. Pecquet, A. Menez, F. Leynadier, and B. Maillere. 2002. Emerging principles for the design of promiscuous HLA-DR-restricted peptides: an example from the major bee venom allergen. Eur J Immunol 32:3699-3707.
14. Vujanovic, L., M. Mandic, W. C. Olson, J. M. Kirkwood, and W. J. Storkus. 2007. A mycoplasma peptide elicits heteroclitic CD4+ T cell responses against tumor antigen MAGE-A6. Clin Cancer Res 13:6796-6806.
15. Zarour, H. M., B. Maillere, V. Brusic, K. Coval, E. Williams, S. Pouvelle-Moratille, F. Castelli, S. Land, J. Bennouna, T. Logan, and J. M. Kirkwood. 2002. NY-ESO-1 119-143 is a promiscuous major histocompatibility complex class II T-helper epitope recognized by Th1- and Th2-type tumor-reactive CD4+ T cells. Cancer Res 62:213-218.
16. Gao, M., H. P. Wang, Y. N. Wang, Y. Zhou, and Q. L. Wang. 2006. HCV-NS3 Th1 minigene vaccine based on invariant chain CLIP genetic substitution enhances CD4(+) Th1 cell responses in vivo. Vaccine 24:5491-5497.
17. Nagata, T., T. Aoshi, M. Suzuki, M. Uchijima, Y. H. Kim, Z. Yang, and Y. Koide. 2002. Induction of protective immunity to Listeria monocytogenes by immunization with plasmid DNA expressing a helper T-cell epitope that replaces the class II-associated invariant chain peptide of the invariant chain. Infect Immun 70:2676-2680.
18. Nagata, T., T. Higashi, T. Aoshi, M. Suzuki, M. Uchijima, and Y. Koide. 2001. Immunization with plasmid DNA encoding MHC class II binding peptide/CLIP-replaced invariant chain (Ii) induces specific helper T cells in vivo: the assessment of Ii p31 and p41 isoforms as vehicles for immunization. Vaccine 20:105-114.
19. Toda, M., M. Kasai, H. Hosokawa, N. Nakano, Y. Taniguchi, S. Inouye, S. Kaminogawa, T. Takemori, and M. Sakaguchi. 2002. DNA vaccine using invariant chain gene for delivery of CD4+ T cell epitope peptide derived from Japanese cedar pollen allergen inhibits allergen-specific IgE response. Eur J Immunol 32:1631-1639.
20. van Bergen, J., M. Camps, R. Offring a, C. J. Melief, F. Ossendorp, and F. Koning. 2000. Superior tumor protection induced by a cellular vaccine carrying a tumor-specific T helper epitope by genetic exchange of the class II-associated invariant chain peptide. Cancer Res 60:6427-6433.
21. van Tienhoven, E. A., C. T. ten Brink, J. van Bergen, F. Koning, W. van Eden, and C. P. Broeren. 2001. Induction of antigen specific CD4+ T cell responses by invariant chain based DNA vaccines. Vaccine 19:1515-1519.

In certain embodiments, the CLIP region of Ii is replaced with a T cell epitope, e.g., a CD4+ T cell epitope, such as a promiscuous CD4+ T cell epitope, with the proviso that the resulting construct is not one that has been publicly disclosed previously, e.g., one year prior to the filing of the priority application of the instant application. For example, in certain embodiments, the epitope that replaces the CLIP region is not a promiscuous CD4+ T cell epitope from an HCV antigen, Listeria LLO antigen, ovalbumin antigen, Japanese cedar pollen allergen, MuLV env/gp70-derived helper epitope, and Heat Shock Protein 60 (described in references 16-21 above), or epitopes replacing CLIP regions that are described in publications that are referenced to in the Examples.

In certain embodiments, a nucleic acid comprises, consists essentially of, or consists of the nucleotide sequence set forth in SEQ ID NO:90, or comprises a nucleotide sequence sequence encoding the PADRE or Ii portion thereof A nucleic acid may also comprise a nucleotide sequence that is at least about 80%, 85%, 90%, 95%, 96%, 97%, 98% or 99% identical to SEQ ID NO:90 and/or to the PADRE and/or to the Ii portion thereof. Nucleic acids may differ by the addition, deletion or substitution of one or more, e.g., 1, 3, 5, 10, 15, 20, 25, 30 or more nucleotides, which may be located at the 5′ end, 3′ end, and/or internally to the sequence.

In certain embodiments, a nucleic acid encodes a protein that is a functional homolog of an Ii-PADRE protein, with the proviso that the Ii sequence and/or PADRE sequence is (or are) not the wild-type or a naturally-occurring sequence, e.g., the wild-type or naturally-occurring human sequence.

In another embodiment, an MHC class I/II activator is a protein that enhances MHC class II expression, e.g., an MHC class II transactivator (CIITA). The nucleotide and amino acid sequences of human CIITA are set forth as GenBank Accession Nos. P33076, NM_{—}000246.3 and NP_{—}000237.2 and set forth as SEQ ID NOs:94 and 95, respectively (GeneID: 4261)).

Variants of the protein may also be used. Exemplary variants comprise, consist essentially of, or consist of an amino acid sequence that is at least about 80%, 85%, 90%, 95%, 96%, 97%, 98% or 99% identical to SEQ ID NO:95. An amino acid sequence may differ from that of SEQ ID NO:95 by the addition, deletion or substitution of at least about 1, 2, 3, 4, 5, 6, 7, 8, 9, 10, 11, 12, 13, 14, 15, 16, 17, 18, 19, 20, 25, 30 or more amino acids. In certain embodiments, a protein lacks one or more, e.g., 2, 3, 4, 5, 6, 7, 8, 9, 10 or more amino acids at the C- and/or N-terminus and/or internally relative to that of SEQ ID NO:95. The locations at which mino acid changes (i.e., deletions, additions or substitutions) may be made may be determined by comparing, i.e., aligning, the amino acid sequences of CIITA homologues, e.g., those from various animal species.

Exemplary amino acids that may be changed include 5286, 5288 and 5293. Indeed, as described in Greer et al., mutation of these amino acids results in a stronger transactivation function relative to the wild-type protein. Changes are preferably not made in the guanine-nucleotide binding motifs within residues 420-561, as these appear to be necessary for CIITA activity (see Chin et al. (1997) PNAS 94:2501). Amino acids 59-94 have also been shown to be necessary for CIITA activity, as further described herein. Additional structure/function data are provided, e.g., in Chin et al., supra.

In certain embodiments, a nucleic acid comprises, consists essentially of, or consists of the nucleotide sequence set forth in SEQ ID NO:94. A nucleic acid may also comprise a nucleotide sequence that is at least about 80%, 85%, 90%, 95%, 96%, 97%, 98% or 99% identical to SEQ ID NO:94. Nucleic acids may differ by the addition, deletion or substitution of one or more, e.g., 1, 3, 5, 10, 15, 20, 25, 30 or more nucleotides, which may be located at the 5′ end, 3′ end, and/or internally to the sequence.

In certain embodiments, a nucleic acid encodes a protein that is a functional homolog of a CIITA protein, with the proviso that the sequence is not the wild-type or a naturally-occurring sequence, e.g., the wild-type or naturally-occurring human sequence.

Other nucleic acids encoding MHC class I/II activators that may be used include those that hybridize, e.g., under stringent hybridization conditions to a nucleic acid encoding an MHC class I/II activator described herein, e.g., consisting of SEQ ID NO:90 or 94 or portions thereof. Hybridization conditions are further described herein.

Nucleic acids encoding an MHC class I/II activator may be included in plasmids or expression vectors, such as those further described herein in the context of DNA vaccines.

In one embodiment, a nucleic acid encoding an Ii-PADRE protein or functional homolog thereof is administered to a subject who is also receiving a nucleic acid encoding a CIITA protein or functional homolog thereof. The nucleic acids may be administered simultaneously or consecutively. The nucleic acids may also be linked, i.e., forming one nucleic acid molecule. For example, one or more nucleotide sequences encoding an Ii-PADRE protein or a functional variant thereof; one or more nucleotide sequences encoding an antigen or a fusion protein comprising an antigen; one or more nucleotide sequences encoding a CITTA protein of a functional variant thereof may be linked to each other, i.e., present on one nucleic acid molecule.

### Chemotherapeutic Drugs

Drugs may also further be administered to a mammal in accordance with the methods and compositions taught herein. Generally, any drug that reduces the growth of cells without significantly affecting the immune system may be used, or at least not suppressing the immune system to the extent of eliminating the positive effects of a DNA vaccine that is administered to the subject. In one embodiment, the drugs are chemotherapeutic drugs.

A wide variety of chemotherapeutic drugs may be used, provided that the drug stimulates the effect of a vaccine, e.g., DNA vaccine. In certain embodiments, a chemotherapeutic drug may be a drug that (a) induces apoptosis of cells, in particular, cancer cells, when contacted therewith; (b) reduces tumor burden; and/or (c) enhances CD8+ T cell-mediated antitumor immunity. In certain embodiments, the drug must also be one that does not inhibit the immune system, or at least not at certain concentrations.

In one embodiment, the chemotherapeutic drug is epigallocatechin-3-gallate (EGCG) or a chemical derivative or pharmaceutically acceptable salt thereof. Epigallocatechin gallate (EGCG) is the major polyphenol component found in green tea. EGCG has demonstrated antitumor effects in various human and animal models, including cancers of the breast, prostate, stomach, esophagus, colon, pancreas, skin, lung, and other sites. EGCG has been shown to act on different pathways to regulate cancer cell growth, survival, angiogenesis and metastasis. For example, some studies suggest that EGCG protects against cancer by causing cell cycle arrest and inducing apoptosis. It is also reported that telomerase inhibition might be one of the major mechanisms underlying the anticancer effects of EGCG. In comparison with commonly-used antitumor agents, including retinoids and doxorubicin, EGCG has a relatively low toxicity and is convenient to administer due to its oral bioavailability. Thus, EGCG has been used in clinical trials and appears to be a potentially ideal antitumor agent.

Exemplary analogs or derivatives of EGCG include (−)-EGCG, (+)-EGCG, (−)-EGCG-amide, (−)-GCG, (+)-GCG, (+)-EGCG-amide, (−)-ECG, (−)-CG, genistein, GTP-1, GTP-2, GTP-3, GTP-4, GTP-5, Bn-(+)-epigallocatechin gallate (US 2004/0186167, incorporated by reference), and dideoxy-epigallocatechin gallate (Furuta, et al., Bioorg. Med. Chem. Letters, 2007, 11: 3095-3098), For additional examples, see US 2004/0186167 (incorporated by reference in its entirety); Waleh, et al., Anticancer Res., 2005, 25: 397-402; Wai, et al., Bioorg. Med. Chem., 2004, 12: 5587-5593; Smith, et al., Proteins: Struc. Func. & Bioinform., 2003, 54: 58-70; U.S. Pat. No. 7,109,236 (incorporated by reference in its entirety); Landis-Piwowar, et al., Int. J. Mol. Med., 2005, 15: 735-742; Landis-Piwowar, et al., J. Cell. Phys., 2007, 213: 252-260; Daniel, et al., Int. J. Mol. Med., 2006, 18: 625-632; Tanaka, et al., Ang. Chemie Int., 2007, 46: 5934-5937.

Another chemotherapeutic drug that may be used is (a) 5,6 di-methylxanthenone-4-acetic acid (DMXAA), or a chemical derivative or analog thereof or a pharmaceutically acceptable salt thereof. Exemplary analogs or derivatives include xanthenone-4-acetic acid, flavone-8-acetic acid, xanthen-9-one-4-acetic acid, methyl (2,2-dimethyl-6-oxo-1,2-dihydro-6H-3,11-dioxacyclopenta[α]anthracen-10-yl)acetate, methyl (2-methyl-6-oxo-1,2-dihydro-6H-3,11-dioxacyclopenta[α]anthracen-10-yl)acetate, methyl (3,3-dimethyl-7-oxo-3H,7H-4,12-dioxabenzo[α]anthracen-10-yl)acetate, methyl-6-alkyloxyxanthen-9-one-4-acetates (Gobbi, et al., 2002, J. Med. Chem., 45: 4931) or a. For additional examples, see WO 2007/023302 A1, WO 2007/023307 A1, US 2006/9505, WO 2004/39363 A1, WO 2003/80044 A1, AU 2003/217035 A1, and AU 2003/282215 A1, each incorporated by reference in their entirety.

A chemotherapeutic drug may also be cisplatin, or a chemical derivative or analog thereof or a pharmaceutically acceptable salt thereof. Exemplary analogs or derivatives include dichloro[4,4′-bis(4,4,4-trifluorobutyl)-2,2′-bipyridine]platinum (Kyler et al., Bioorganic & Medicinal Chemistry, 2006, 14: 8692-8700), cis-[Rh2(—O2CCH3)2(CH3CN)6]2+ (Lutterman et al., J. Am. Chem. Soc., 2006, 128: 738-739), (+)-cis-(1,1-Cyclobutanedicarboxylato)((2R)-2-methyl-1,4-butanediamine-N,N′)platinum (O'Brien et al., Cancer Res., 1992, 52: 4130-4134), cis-bisneodecanoato-trans-R,R-1,2-diaminocyclohexane platinum(II) (Lu et al., J. of Clin. Oncol., 2005, 23: 3495-3501), carboplatin (Woloschuk, Drug Intell. Clin. Pharm., 1988, 22: 843-849), sebriplatin (Kanazawa et al., Head & Neck, 2006, 14: 38-43), satraplatin (Amorino et al., Cancer Chemother. and Pharmacol., 2000, 46: 423-426), azane (dichloroplatinum) (CID: 11961987), azanide (CID: 6712951), platinol (CID: 5702198), lopac-P-4394 (CID: 5460033), MOLI001226 (CID: 450696), trichloroplatinum (CID: 420479), platinate(1-), amminetrichloro-, ammonium (CID: 160995), triammineplatinum (CID: 119232), biocisplatinum (CID: 84691), platiblastin (CID: 2767) and pharmaceutically acceptable salts thereof. For additional examples, see U.S. Pat. No. 5,922,689, U.S. Pat. No. 4,996,337, U.S. Pat. No. 4,937,358, U.S. Pat. No. 4,808,730, U.S. Pat. No. 6,130,245, U.S. Pat. No. 7,232,919, and U.S. Pat. No. 7,038,071, each incorporated by reference in their entirety.

Another chemotherapeutic drug that may be used is apigenin, or a chemical derivative or analog thereof or a pharmaceutically acceptable salt thereof. Exemplary analogs or derivatives include acacetin, chrysin, kampherol, luteolin, myricetin, naringenin, quercetin (Wang et al., Nutrition and Cancer, 2004, 48: 106-114), puerarin (US 2006/0276458, incorporated by reference in its entirety) and pharmaceutically acceptable salts thereof. For additional examples, see US 2006/189680 A1, incorporated by reference in its entirety).

Another chemotherapeutic drug that may be used is doxorubicin, or a chemical derivative or analog thereof or a pharmaceutically acceptable salt thereof. Exemplary analogs or derivatives include anthracyclines, 3′-deamino-3′-(3-cyano-4-morpholinyl)doxorubicin, WP744 (Faderl, et al., Cancer Res., 2001, 21: 3777-3784), annamycin (Zou, et al., Cancer Chemother. Pharmacol., 1993, 32:190-196), 5-imino-daunorubicin, 2-pyrrolinodoxorubicin, DA-125 (Lim, et al., Cancer Chemother. Pharmacol., 1997, 40: 23-30), 4-demethoxy-4′-O-methyldoxorubicin, PNU 152243 and pharmaceutically acceptable salts thereof (Yuan, et al., Anti-Cancer Drugs, 2004, 15: 641-646). For additional examples, see EP 1242438 B1, U.S. Pat. No. 6,630,579, AU 2001/29066 B2, U.S. Pat. No. 4,826,964, U.S. Pat. No. 4,672,057, U.S. Pat. No. 4,314,054, AU 2002/358298 A1, and U.S. Pat. No. 4,301,277, each incorporated by reference in their entirety);

Other chemotherapeutic drugs that may be used are anti-death receptor 5 antibodies and binding proteins, and their derivatives, including antibody fragments, single-chain antibodies (scFvs), Avimers, chimeric antibodies, humanized antibodies, human antibodies and peptides binding death receptor 5. For examples, see US 2007/31414 and US 2006/269554, each incorporated by reference in their entirety.

Another chemotherapeutic drug that may be used is bortezomib, or a chemical derivative or analog thereof or a pharmaceutically acceptable salt thereof. Exemplary analogs or derivatives include MLN-273 and pharmaceutically acceptable salts thereof (Witola, et al., Eukaryotic Cell, 2007, doi:10.1128/EC.00229-07). For additional possibilities, see Groll, et al., Structure, 14:451.

Another chemotherapeutic drug that may be used is 5-aza-2-deoxycytidine, or a chemical derivative or analog thereof or a pharmaceutically acceptable salt thereof. Exemplary analogs or derivatives include other deoxycytidine derivatives and other nucleotide derivatives, such as deoxyadenine derivatives, deoxyguanine derivatives, deoxythymidine derivatives and pharmaceutically acceptable salts thereof.

Another chemotherapeutic drug that may be used is genistein, or a chemical derivative or analog thereof or a pharmaceutically acceptable salt thereof. Exemplary analogs or derivatives include 7-O-modified genistein derivatives (Zhang, et al., Chem. & Biodiv., 2007, 4: 248-255), 4′,5,7-tri[3-(2-hydroxyethylthio)propoxy]isoflavone, genistein glycosides (Polkowski, Cancer Letters, 2004, 203: 59-69), other genistein derivatives (L1, et al., Chem & Biodiv., 2006, 4: 463-472; Sarkar, et al., Mini. Rev. Med. Chem., 2006, 6: 401-407) or pharmaceutically acceptable salts thereof. For additional examples, see U.S. Pat. No. 6,541,613, U.S. Pat. No. 6,958,156, and WO/2002/081491, each incorporated by reference in their entirety.

Another chemotherapeutic drug that may be used is celecoxib, or a chemical derivative or analog thereof or a pharmaceutically acceptable salt thereof. Exemplary analogs or derivatives include N-(2-aminoethyl)-4-[5-(4-tolyl)-3-(trifluoromethyl)-1H-pyrazol-1-yl]benzenesulfonamide, 4-[5-(4-aminophenyl)-3-(trifluoromethyl)-1H-pyrazol-1-yl]benzenesulfonamide, OSU03012 (Johnson, et al., Blood, 2005, 105: 2504-2509), OSU03013 (Tong, et. al, Lung Cancer, 2006, 52: 117-124), dimethyl celecoxib (Backhus, et al., J. Thorac. and Cardiovasc. Surg., 2005, 130: 1406-1412), and other derivatives or pharmaceutically acceptable salts thereof (Ding, et al., Int. J. Cancer, 2005, 113: 803-810; Zhu, et al., Cancer Res., 2004, 64: 4309-4318; Song, et al., J. Natl. Cancer Inst., 2002, 94: 585-591). For additional examples, see U.S. Pat. No. 7,026,346, incorporated by reference in its entirety.

One of skill in the art will readily recognize that other chemotherapeutics can be used with the methods disclosed in the present invention, including proteasome inhibitors (in addition to bortezomib) and inhibitors of DNA methylation. Other drugs that may be used include Paclitaxel; selenium compounds; SN38, etoposide, 5-Fluorouracil; VP-16, cox-2 inhibitors, Vioxx, cyclooxygenase-2 inhibitors, curcumin, MPC-6827, tamoxifen or flutamide, etoposide, PG490, 2-methoxyestradiol, AEE-788, aglycon protopanaxadiol, aplidine, ARQ-501, arsenic trioxide, BMS-387032, canertinib dihydrochloride, canfosfamide hydrochloride, combretastatin A-4 prodrug, idronoxil, indisulam, INGN-201, mapatumumab, motexafin gadolinium, oblimersen sodium, OGX-011, patupilone, PXD-101, rubitecan, tipifarnib, trabectedin PXD-101, methotrexate, Zerumbone, camptothecin, MG-98, VX-680, Ceflatonin, Oblimersen sodium, motexafin gadolinium, 1D09C3, PCK-3145, ME-2 and apoptosis-inducing-ligand (TRAIL/Apo-2 ligand). Others are provided in a report entitled “competitive outlook on apoptosis in oncology, December 2006, published by Bioseeker, and available, e.g., at http://bizwiz.bioseeker.com/bw/Archives/Files/TOC_BSG0612193.pdf.

Generally, any drug that affects an apoptosis target may also be used. Apoptosis targets include the tumour-necrosis factor (TNF)-related apoptosis-inducing ligand (TRAIL) receptors, the BCL2 family of anti-apoptotic proteins (such as Bc1-2), inhibitor of apoptosis (IAP) proteins, MDM2, p53, TRAIL and caspases. Exemplary targets include B-cell CLL/lymphoma 2, Caspase 3, CD4 molecule, Cytosolic ovarian carcinoma antigen 1, Eukaryotic translation elongation factor 2, Farnesyltransferase, CAAX box, alpha; Fc fragment of IgE; Histone deacetylase 1; Histone deacetylase 2; Interleukin 13 receptor, alpha 1; Phosphodiesterase 2A, cGMP-stimulatedPhosphodiesterase 5A, cGMP-specific; Protein kinase C, beta 1; Steroid 5-alpha-reductase, alpha polypeptide 1; 8.1.15 Topoisomerase (DNA) I; Topoisomerase (DNA) II alpha; Tubulin, beta polypeptide; and p53 protein.

In certain embodiments, the compounds described herein, e.g., EGCG, are naturally-occurring and may, e.g., be isolated from nature. Accordingly, in certain embodiments, a compound is used in an isolated or purified form, i.e., it is not in a form in which it is naturally occurring. For example, an isolated compound may contain less than about 50%, 30%, 10%, 1%, 0.1% or 0.01% of a molecule that is associated with the compound in nature. A purified preparation of a compound may comprise at least about 50%, 70%, 80%, 90%, 95%, 97%, 98% or 99% of the compound, by molecule number or by weight. Compositions may comprise, consist essentially of consist of one or more compounds described herein. Some compounds that are naturally occurring may also be synthesized in a laboratory and may be referred to as “synthetic.” Yet other compounds described herein are non-naturally occurring.

In certain embodiments, the chemotherapeutic drug is in a preparation from a natural source, e.g., a preparation from green tea.

Pharmaceutical compositions comprising 1, 2, 3, 4, 5 or more chemotherapeutic drugs or pharmaceutically acceptable salts thereof are also provided herein. A pharmaceutical composition may comprise a pharmaceutically acceptable carrier. A composition, e.g., a pharmaceutical composition, may also comprise a vaccine, e.g., a DNA vaccine, and optionally 1, 2, 3, 4, 5 or more vectors, e.g., other DNA vaccines or other constructs, e.g., described herein.

Compounds may be provided with a pharmaceutically acceptable salt. The term “pharmaceutically acceptable salts” is art-recognized, and includes relatively non-toxic, inorganic and organic acid addition salts of compositions, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66:1-19 (1977).

Also provided herein are compositions and kits comprising one or more DNA vaccines and one or more chemotherapeutic drugs, and optionally one or more other constructs described herein.

Therapeutic Compositions and their Administration

The methods of the present invention can be practiced by administering papillomavirus pseudovirions described herein in a pharmaceutically acceptable carrier in a biologically-effective and/or a therapeutically-effective amount.

Certain conditions as described herein are disclosed in the Examples. The composition may be given alone or in combination with another protein or peptide such as an immunostimulatory molecule. Treatment may include administration of an adjuvant, used in its broadest sense to include any nonspecific immune stimulating compound such as an interferon. Adjuvants contemplated herein include resorcinols, non-ionic surfactants such as polyoxyethylene oleyl ether and n-hexadecyl polyethylene ether.

A therapeutically effective amount is a dosage that, when given for an effective period of time, achieves the desired immunological or clinical effect.

A therapeutically active amount of a nucleic acid encoding the fusion polypeptide may vary according to factors such as the disease state, age, sex, and weight of the individual, and the ability of the peptide to elicit a desired response in the individual. Dosage regimes may be adjusted to provide the optimum therapeutic response. For example, several divided doses may be administered daily or the dose may be proportionally reduced as indicated by the exigencies of the therapeutic situation. A therapeutically effective amount of the protein, in cell associated form may be stated in terms of the protein or cell equivalents.

Thus an effective amount of the papillomavirus pseudovirions may be between about 1 nanogram and about 1 gram per kilogram of body weight of the recipient, between about 0.1 μg/kg and about 10 mg/kg, between about 1 μg/kg and about 1 mg/kg. Dosage forms suitable for internal administration may contain (for the latter dose range) from about 0.1 μg to 100 μg of active ingredient per unit. The active ingredient may vary from 0.5 to 95% by weight based on the total weight of the composition. Alternatively, an effective dose of cells transfected with the DNA vaccine constructs of the present invention is between about 10⁴ and 10⁸ cells. Those skilled in the art of immunotherapy will be able to adjust these doses without undue experimentation.

Embodiments disclosed herein also relate to methods of administering papillomavirus pseudovirions described herein to a subject in order to contact in vivo cells with such compositions. The routes of administration can vary with the location and nature of the cells to be contacted, and include, e.g., intravascular, intradermal, transdermal, parenteral, intravenous, intramuscular, intranasal, subcutaneous, regional, percutaneous, intratracheal, intraperitoneal, intraarterial, intravesical, intratumoral, inhalation, perfusion, lavage, direct injection, and oral administration and formulation. In other embodiments, the routes of administration of the DNA may include (a) intratumoral, peritumoral, and/or intradermal delivery, (b) intramuscularly (i.m.) injection using a conventional syringe needle; and (c) use of a needle-free biojector such as the Biojector 2000 (Bioject Inc., Portland, Oreg.) which is an injection device consisting of an injector and a disposable syringe. The orifice size controls the depth of penetration. For example, 50 μg of DNA may be delivered using the Biojector with no. 2 syringe nozzle.

The term “systemic administration” refers to administration of a composition or agent such as a DNA vaccine as described herein, in a manner that results in the introduction of the composition into the subject's circulatory system or otherwise permits its spread throughout the body. “Regional” administration refers to administration into a specific, and somewhat more limited, anatomical space, such as intraperitoneal, intrathecal, subdural, or to a specific organ. “Local administration” refers to administration of a composition or drug into a limited, or circumscribed, anatomic space, such as intratumoral injection into a tumor mass, subcutaneous injections, intradermal or intramuscular injections. Those of skill in the art will understand that local administration or regional administration may also result in entry of a composition into the circulatory system i.e., rendering it systemic to one degree or another. For example, the term “intravascular” is understood to refer to delivery into the vasculature of a patient, meaning into, within, or in a vessel or vessels of the patient, whether for systemic, regional, and/or local administration. In certain embodiments, the administration can be into a vessel considered to be a vein (intravenous), while in others administration can be into a vessel considered to be an artery. Veins include, but are not limited to, the internal jugular vein, a peripheral vein, a coronary vein, a hepatic vein, the portal vein, great saphenous vein, the pulmonary vein, superior vena cava, inferior vena cava, a gastric vein, a splenic vein, inferior mesenteric vein, superior mesenteric vein, cephalic vein, and/or femoral vein. Arteries include, but are not limited to, coronary artery, pulmonary artery, brachial artery, internal carotid artery, aortic arch, femoral artery, peripheral artery, and/or ciliary artery. It is contemplated that delivery may be through or to an arteriole or capillary.

Injection into the tumor vasculature is specifically contemplated for discrete, solid, accessible tumors. Local, regional or systemic administration also may be appropriate. For tumors of greater than about 4 cm, the volume to be administered can be about 4-10 ml (preferably 10 ml), while for tumors of less than about 4 cm, a volume of about 1-3 ml can be used (preferably 3 ml). Multiple injections delivered as single dose comprise about 0.1 to about 0.5 ml volumes. The pseudoviruses may advantageously be contacted by administering multiple injections to the tumor, spaced at approximately 1 cm intervals.

Continuous administration also may be applied where appropriate, for example, where a tumor is excised and the tumor bed is treated to eliminate residual, microscopic disease. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs. Other routes of administration include oral, intranasal or rectal or any other route known in the art.

Depending on the route of administration, the composition may be coated in a material to protect the compound from the action of enzymes, acids and other natural conditions which may inactivate the compound. Thus it may be necessary to coat the composition with, or co-administer the composition with, a material to prevent its inactivation. For example, an enzyme inhibitors of nucleases or proteases (e.g., pancreatic trypsin inhibitor, diisopropylfluorophosphate and trasylol) or in an appropriate carrier such as liposomes (including water-in-oil-in-water emulsions as well as conventional liposomes (Strejan et al., J. Neuroimmunol 7:27, 1984).

A chemotherapeutic drug may be administered in doses that are similar to the doses that the chemotherapeutic drug is used to be administered for cancer therapy. Alternatively, it may be possible to use lower doses, e.g., doses that are lower by 10%, 30%, 50%, or 2, 5, or 10 fold lower. Generally, the dose of chemotherapeutic agent is a dose that is effective to increase the effectiveness of a DNA vaccine, but less than a dose that results in significant immunosuppression or immunosuppression that essentially cancels out the effect of the DNA vaccine.

The route of administration of chemotherapeutic drugs may depend on the drug. For use in the methods described herein, a chemotherapeutic drug may be used as it is commonly used in known methods. Generally, the drugs will be administered orally or they may be injected. The regimen of administration of the drugs may be the same as it is commonly used in known methods. For example, certain drugs are administered one time, other drugs are administered every third day for a set period of time, yet other drugs are administered every other day or every third, fourth, fifth, sixth day or weekly. The Examples provide exemplary regimens for administrating the drugs, as well as DNA vaccines.

The compositions of the present invention, may be administered simultaneously or subsequently. When administered simultaneously, the different components may be administered as one composition. Accordingly, also provided herein are compositions, e.g., pharmaceutical compositions comprising one or more agents.

In one embodiment, a subject first receives one or more doses of chemotherapeutic drug and then one or more doses of DNA vaccine. In the case of DMXAA, it may be preferable to administer to the subject a dose of DNA vaccine first and then a dose of chemotherapeutic drug. One may administer 1, 2, 3, 4, 5 or more doses of DNA vaccine and 1, 2, 3, 4, 5 or more doses of chemotherapeutic agent.

A method may further comprise subjecting a subject to another cancer treatment, e.g., radiotherapy, an anti-angiogenesis agent and/or a hydrogel-based system.

As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

Pharmaceutically acceptable diluents include saline and aqueous buffer solutions. Pharmaceutical compositions suitable for injection include sterile aqueous solutions (where water soluble) or dispersions and sterile powders for the extemporaneous preparation of sterile injectable solutions or dispersion. Isotonic agents, for example, sugars, polyalcohols such as mannitol, sorbitol, sodium chloride may be included in the pharmaceutical composition. In all cases, the composition should be sterile and should be fluid. It should be stable under the conditions of manufacture and storage and must include preservatives that prevent contamination with microorganisms such as bacteria and fungi. Dispersions can also be prepared in glycerol, liquid polyethylene glycols, and mixtures thereof and in oils. Under ordinary conditions of storage and use, these preparations may contain a preservative to prevent the growth of microorganisms.

The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms in the pharmaceutical composition can be achieved by various antibacterial and antifungal agents, for example, parabens, chlorobutanol, phenol, ascorbic acid, thimerosal, and the like.

Compositions may be formulated in dosage unit form for ease of administration and uniformity of dosage. Dosage unit form refers to physically discrete units suited as unitary dosages for a mammalian subject; each unit contains a predetermined quantity of active material (e.g., the nucleic acid vaccine) calculated to produce the desired therapeutic effect, in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active compound for the treatment of, and sensitivity of, individual subjects. Unit dose of the present invention may conveniently be described in terms of plaque forming units (pfu) for a viral construct. Unit doses range from 10³, 10⁴, 10⁵, 10⁶, 10⁷, 10⁸, 10⁹, 10¹⁰, 10¹¹, 10¹², and 10¹³ pfu and higher. Alternatively, depending on the type of papillomavirus pseudovirion and the titer attainable, one will deliver 1 to 100, 10 to 50, 100-1000, or up to about 10⁴, 10⁵, 10⁶, 10⁷, 10⁸, 10⁹, 10¹⁰, 10¹¹, 10¹², 10¹³, 10¹⁴, and 10¹⁵ pfu or higher infectious papillomavirus pseudovirions to the subject or to the patient's cells.

For lung instillation, aerosolized solutions are used. In a sprayable aerosol preparations, the active protein may be in combination with a solid or liquid inert carrier material. This may also be packaged in a squeeze bottle or in admixture with a pressurized volatile, normally gaseous propellant. The aerosol preparations can contain solvents, buffers, surfactants, and antioxidants in addition to the protein of the invention.

Diseases that may be treated as described herein include hyper proliferative diseases, e.g., cancer, whether localized or having metastasized. Exemplary cancers include head and neck cancers and cervical cancer. Any cancer can be treated provided that there is a tumor associated antigen that is associated with the particular cancer. Other cancers include skin cancer, lung cancer, colon cancer, kidney cancer, breast cancer, prostate cancer, pancreatic cancer, bone cancer, brain cancer, as well as blood cancers, e.g., myeloma, leukemia and lymphoma. Generally, any cell growth can be treated provided that there is an antigen associated with the cell growth, which antigen or homolog thereof can be encoded by a DNA vaccine.

Treating a subject includes curing a subject or improving at least one symptom of the disease or preventing or reducing the likelihood of the disease to return. For example, treating a subject having cancer could be reducing the tumor mass of a subject, e.g., by about 10%, 30%, 50%, 75%, 90% or more, eliminating the tumor, preventing or reducing the likelihood of the tumor to return, or partial or complete remission.

All references cited herein are all incorporated by reference herein, in their entirety, whether specifically incorporated or not. All publications, patents, patent applications, GenBank sequences and ATCC deposits, cited herein are hereby expressly incorporated by reference for all purposes. In particular, all nucleotide sequences, amino acid sequences, nucleic constructs, DNA vaccines, methods of administration, particular orders of administration of DNA vaccines and agents that are described in the patents, patent applications and other publications referred to herein or authored by one or more of the inventors of this application are specifically incorporated by reference herein. In case of conflict, the definitions within the instant application govern.

Having now fully described this invention, it will be appreciated by those skilled in the art that the same can be performed within a wide range of equivalent parameters, concentrations, and conditions without departing from the spirit and scope of the invention and without undue experimentation.

The present description is further illustrated by the following examples, which should not be construed as limiting in any way.

### EXAMPLES

### Example 1

### Material and Methods For Examples 2-7

### A. Mice

C57BL/6 mice (5- to 8-week-old) were purchased from the National Cancer Institute (Frederick, Md.). OT-1 transgenic mice on C57BL/6 background were purchased from Taconic. All animals were maintained under specific-pathogen free conditions, and all procedures were performed according to approved protocols and in accordance with recommendations for the proper use and care of laboratory animals.

### B. Peptides, Antibodies and Reagents

The H-2K^{b}-restricted Ovalbumin (OVA) peptide, SIINFEKL (SEQ ID NO: 118) was synthesized by Macromolecular Resources (Denver, Colo.) at a purity of ≧80%. FITC-conjugated rat anti-mouse IFN-γ, PE-conjugated anti-mouse CD8, PE-Cy5 conjugated anti-mouse B220 and APC-conjugated anti-mouse CD11c antibodies were purchased from BD Pharmingen (BD Pharmingen, San Diego, Calif.). A horse radish peroxidase-conjugated rabbit anti-mouse immunoglobulin G (IgG) antibody was purchased from Zymed (San Francisco, Calif.). OVA protein was purchased from Sigma.

### C. Plasmid DNA Constructs

293TT cells were kindly provided by J. Schiller (NCI, NIH) (Buck et al., J. Virol., 78:751-757 (2004)). These cells were generated by transfecting 293T cells with an additional copy of the SV40 large T antigen. Murine melanoma cell line, B 16 expressing OVA was described in Chuang et al., Clin. Cancer Res., 15:4581-4588 (2009). Both cell lines were grown in complete Dulbecco's modified Eagle medium (DMEM) (Invitrogen) containing 10% heat-inactivated fetal bovine serum (Gemini Bio-Products). The immortalized DC line was provided by Dr. K. Rock (University of Massachusetts, Worcester, Mass.) (Shen et al., J. Immunol., 158:2723-2730 (1997)). With continued passage, subclones of the DC line, DC-1, were generated that are easily transfected using Lipofectamine 2000 (Invitrogen) (Kim et al., Cancer Res., 64:400-405 (2004)). The EG.7 cell line, derived from murine EL4 lymphoma cell transfected with OVA-expressing vector was purchased from ATCC. Both DC-1 and EG.7 cells were cultured in complete RPMI-1640 medium containing 10% heat-inactivated fetal bovine serum. The OVA peptide, SIINFEKL (SEQ ID NO: 118)-specific CD8 T cell line was generated by stimulating splenocytes from OT-1 transgenic mice with irradiated EG.7 cells in the presence of IL-2 (20 IU/ml, Pepro-Tech).

### D. Plasmid Construction

The plasmids encoding HPV16 and 18 L1 and L2 (pShe1116, pShe1118, p16L1 and p16L2) were kindly provided by Dr. John Schiller (NCI). The point mutation HPV16L1mtL2-OVA construct was described in Gambhira et al. Virol. J, 6:176 (2009). The generation of ovalbumin-expressing plasmid (pcDNA3-OVA) and GFP-expressing plasmid (pcDNA3-GFP) was described in Kim et al., J. Clin. Invest., 112:109-117 (2003) and Hung et al., Cancer Res., 61:3698-3703 (2001).

### E. HPV Pseudovirion Production

HPV16 and HPV18 pseudovirions were made as described in Buck et al., J. Virol., 78:751-757 (2004). Briefly, 293TT cells were co-transfected with HPV L1 and L2 expression plasmids and the targeted antigen-expressing plasmids using Lipofectamine 2000 (Invitrogen, Carlsbad, Calif.). After 48 hours, the cells were harvested and washed with Dulbecco's PBS (Invitrogen) supplemented with 9.5 mM MgCl₂ and antibiotic-antimycotic mixture (DPBS-Mg) (Invitrogen). The cells were suspended in DPBS-Mg supplemented with 0.5% Briji58, 0.2% Benzonase (Novagen), 0.2% Plasmid Safe (Epicentre) at >100×10⁶ cells/ml and incubated at 37° C. for 24 hours for capsid maturation. After maturation, the cell lysate was chilled on ice for 10 minutes. The salt concentration of the cell lysate was adjusted to 850 mM and incubated on ice for 10 minutes. The lysate was then clarified by centrifugation, and the supernatant was then layered onto an Optiprep gradient. The gradient was spun for 4.5 hours at 16° C. at 40,000 rpm in a SW40 rotor (Beckman). Furin-precleaved pseudovirion (FPC) was produced as described in Day et al., J. Virol., 82:12565-12568 (2008). Briefly, 20 U/ml of furin was added to the pseudovirion extract prior to the maturation process. After maturation, the FPC virions were purified as described above. The purity of HPV pseudovirions was evaluated by running the fractions on 4-15% gradient SDS-PAGE gel. The encapsulated DNA plasmid was quantified by extracting encapsidated DNA from Optiprep factions followed by quantitative real time PCR compared to serial dilutions of naked DNA.

### F. Characterization of the Amount of DNA Contained in Pseudovirions

The extraction of plasmid DNA from pseudovirions for the quantitative real-time PCR was performed using methods from John Schiller's Group (Laboratory of Cellular Oncology, NCI). Briefly, 100 μl of Optiprep fraction material adding 10 μl of 0.5M EDTA and 2.5 μl of proteinase K (Qiagen) was incubated at 56° C. for 30 minutes followed adding 5 μl of 10% SDS and another incubation 30 min. After incubation, the solution was massed up 200 μl and 200 μl of equilibrated phenol-chloroform-isoamylalcohol (Roche) and 200 μl of chloroform-isoamylalcohol (Sigma) was used serially for the preparation of extracted lysate. 2.6 volumes of 95% ethanol were added to about 200 μl of extracted lysate and precipitate DNA 4° C. overnight. After spin down for 60 min at 15,000×g room temperature, supernatant was removed carefully. Pellet was washed with 800 μl of 70% ethanol and dissolved in 50 μl of dH₂O. For quantifying plasmid DNA, quantitative real-time PCR reactions were performed in triplicates using Bio-Rad iCycler. OVA or No insert plasmid DNA from pseudovirus and naked OVA or No insert were used as a template for amplification using primers for OVA or No insert (OVA: 5′-AATGGACCAGTTCTAATGT-3′ (SEQ ID NO:110), 5′-GTCAGCCCTAAATTCTTC-3′ (SEQ ID NO:111) or No insert: 5′-TAATACGACTCACTATAGGG-3′ (SEQ ID NO:112), 5′-TAGAAGGCACAGTCGAGG-3′ (SEQ ID NO:113)) and amplified products were quantified by fluorescence intensity of SYBR Green I (Molecular Probes). A standard curve method was used to calculate the quantity of pseudovirus plasmid DNA relative to the naked plasmid DNA. Five serial dilutions of naked plasmid (OVA or No insert) were made for the calibration curve and trend lines were drawn using Ct values versus log of dilutions for each plasmid. The quantity of pseudovirus plasmid DNA was calculated using line equations derived from calibration curves. The concentration of pcDNA3 plasmid DNA and pcDNA3-OVA DNA in the pseudovirions was determined to be approx. 6.2 ng of DNA per 1 μg of L1 protein.

### G. HPV Pseudovirions Labeling and In Vivo Uptake

HPV 16-OVA pseudovirions were labeled with FITC using the FluoReporter FITC protein labeling kit (F6434) (Invitrogen). After extensive washing, FITC labeled or unlabeled pseudovirions were injected into the hind footpads of mouse. 48 hours later, inguinal and popliteal lymph nodes were collected, minced and digested with 0.05 mg/ml Collagenase I, 0.05 mg/ml collagenase IV, 0.025 mg/ml Hyaluronidase IV (Sigma) and 0.25 mg/ml DNase I (Roche) at 37° C. for 1 hour. After washing, the cells were stained with anti-mouse B220 and CD11c antibody, labeled with FITC and analyzed with flow cytometry.

### H. Generation of Bone Marrow-Derived Dendritic Cells

Bone marrow-derived dendritic cells (BMDCs) were generated from bone marrow progenitor cells as described in Peng et al., Hum. Gene Ther., 16:584-593 (2005). Briefly, bone marrow cells were flushed from the femurs and tibiae of 5- to 8-week-old C57BL/6 mice. Cells were washed twice with RPMI-1640 after lysis of red blood cells and resuspended at a density of 1×10⁶/ml in RPMI-1640 medium supplemented with 2 mM glutamine, 1 mM sodium pyruvate, 100 mM nonessential amino acids, 55 μM β-mercaptoethanol, 100 IU/ml penicillin, 100 g/ml streptomycin, 5% fetal bovine serum, and 20 ng/ml recombinant murine GM-CSF (PeproTech, Rock Hill, N.J.). The cells were then cultured in a 24-well plate (1 ml/well) at 37° C. in 5% humidified CO₂. The wells were replenished with fresh medium supplemented with 20 ng/ml recombinant murine GM-CSF on days 2 and 4. The cells were harvested as indicated.

I. In Vitro Infection with HPV Pseudovirions

DC-1 cells were seeded into 24-well plate at the density of 1×10⁵/well, and infected with 5 (HPV L1 protein amount) of HPV16-GFP or HPV 16-OVA pseudovirions. For furin cleavage experiment, 5 units/ml of furin (Alexis Biochemical, San Diego) were added to the cell culture medium. BMDCs were also infected with 5 (HPV L1 protein amount) of HPV16-GFP or HPV 16-OVA pseudovirions. 72 hours later, the cells were analyzed for GFP expression by flow cytometry or used in T cell activation assay.

### J. In Vitro T Cell Activation Assay

OT-1 T cells were co-incubated with HPV16-GFP or HPV16-OVA pseudovirions infected DC-1 cells (E:T ratio 2:1) at the presence of GolgiPlug (BD Pharmingen) at 37° C. for 20 hours. T cell activation was analyzed by detecting intracellular IFN-γ production with flow cytometry analysis.

K. Vaccination with HPV Pseudovirions

C57BL/6 mice were vaccinated with indicated HPV pseudovirions (adjusted to 5 μg L1 protein amount) at both hind footpads. 7 days later, the mice were boosted with indicated HPV pseudovirions with the same regimen. For antibody detection experiment, sera were collected before and after vaccination at indicated time point. For antigen-specific T cell detection, mouse splenocytes were harvested 1 week after last vaccination.

### L. DNA Vaccination

Gene gun particle-mediated DNA vaccination was performed as described in Peng et al., J. Virol., 78:8468-8476 (2004). Gold particles coated with pcDNA3-OVA, or pcDNA3 were delivered to the shaved abdominal regions of mice by using a helium-driven gene gun (Bio-Rad Laboratories Inc., Hercules, Calif.) with a discharge pressure of 400 lb/in². Mice were immunized with 2 μg of the DNA vaccine and boosted with the same regimen 1 week later. Splenocytes were harvested 1 week after the last vaccination.

### M. Antibody Neutralization Assays

The HPV pseudovirion in vitro neutralization assay was performed as described in Pastrana et al., Virology, 321:205-216 (2004), and the secreted alkaline phosphatase activity in the cell-free supernatant was determined using p-nitrophenyl phosphate (Sigma Aldrich, St Louis, Mo.) dissolved in diethanolamine, with absorbance measured at 405 nm. Neutralizing antibody titers were defined as the reciprocal of the highest dilution that caused a greater than 50% reduction in A₄₀₅, as described in Pastrana et al., Virology, 321:205-216 (2004). Pre-immune sera were used as a negative control and mouse monoclonal antibody RG-1 or rabbit antiserum to L1 VLP as positive controls (Jagu et al., J. Natl. Cancer Inst., 101:782-792 (2009)).

### N. Detection of Ovalbumin-Specific Antibody by ELISA

To detect OVA-specific antibody in vaccinated mouse sera, an ELISA assay was performed. Briefly, maximum absorption 96-well ELISA plate was coated with OVA protein (Sigma) at 1 μg/ml, and incubated at 4° C. overnight. After blocking with PBS containing 1% BSA for 1 h at 37° C., the wells were then washed with PBS containing 0.05% Tween-20. The plate was incubated with serially diluted sera for 2 h at 37° C. Serum from mouse vaccinated with OVA protein via intramuscular injection plus electroporation (Kang T H, et al. manuscript in preparation) was used as the positive control. After washing with PBS containing 0.05% Tween-20, the plate was further incubated with 1:2,000 dilution of a HRP-conjugated rabbit anti-mouse IgG antibody (Zymed, San Francisco, Calif.) at room temperature for 1 h. The plate was washed, developed with 1-Step Turbo TMB-ELISA (Pierce, Rockford, Ill.), and stopped with 1 M H₂SO₄. The ELISA plate was read with a standard ELISA reader at 450 nm.

### O. Intracellular Cytokine Staining and Flow Cytometry Analysis

Before intracellular cytokine staining, pooled splenocytes from each vaccination group were incubated for 20 hours with 1 μg/ml of OVA SIINFEKL (SEQ ID NO: 118) peptide at the presence of GolgiPlug (BD Pharmingen, San Diego, Calif.). The stimulated splenocytes were then washed once with FACScan buffer and stained with PE-conjugated monoclonal rat antimouse CD8a (clone 53.6.7). Cells were subjected to intracellular cytokine staining using the Cytofix/Cytoperm kit according to the manufacturer's instruction (BD Pharmingen, San Diego, Calif.). Intracellular IFN-γ was stained with FITC-conjugated rat anti-mouse IFN-γ (clone XMG1.2). Flow cytometry analysis was performed using FACSCalibur with CELLQuest software (BD biosciences, Mountain View, Calif.).

### P. RT-PCR Analysis of In Vivo GFP Expression

To detect GFP expression in the draining lymph nodes after pseudovirion infection, total RNA was extracted from draining lymph nodes 48 hours after subcutaneous HPV 16-GFP or HPV16-OVA pseudovirions infection. RT-PCR was performed as described in Kim et al., J. Biomed. Sci., 11:493-499 (2004). Briefly, the RNA was extracted from the cells by TRIZOL (Invitrogen, Carlsbad, Calif.). RT-PCR was performed using the Superscript One-Step RT-PCR Kit (Invitrogen). One microgram of total RNA was used. Sequences of primers for GFP and GAPDH were as follows: GFP-F (5′-ATGGTGAGCAAGGGCGAGGAG-3′ (SEQ ID NO:114)), GFP-R (5′-CTTGTACAGCTCGTCCATGCC-3′ (SEQ ID NO:115)), GAPDH-F (5′-CCGGATCCTGGGAAGCTTGTCATCAACGG-3′ (SEQ ID NO:116)), and GAPDH-R (5′-GGCTCGAGGCAGTGATGGCATGGACTG-3′ (SEQ ID NO:117)). The reaction condition for GFP was 1 cycle (94° C., 30 sec), 35 cycle (94° C., 30 sec; 55° C., 30 sec; 72° C., 30 sec), and 1 cycle (72° C., 10 min). The reaction condition for GAPDH was similar except that amplification was repeated for 20 cycles. The products were analysed by electrophoresis on a 1.5% agarose gel containing ethidium bromide. GAPDH expression was used as positive control and no RT was used as a negative control.

### Q. In Vivo Tumor Protection and Antibody Depletion

C57BL/6 mice (five per group) were vaccinated with the indicated HPV pseudovirions (adjusted with 5 μg L1 protein amount) at both hind footpads. 7 days later, the mice were boosted with indicated HPV pseudovirions with the same regimen. 1 week after last vaccination, mice were injected with 1×10⁵ B16-OVA tumor cells subcutaneously at the flank site in 100 μL PBS. In vivo antibody depletions have been described previously (Lin et al., Cancer Res., 56:21-26 (1996)). Briefly, monoclonal antibody (MAb) GK1.5 was used for CD4 depletion, MAb 2.43 was used for CD8 depletion and MAb PK136 was used for NK1.1 depletion. Depletion started 1 week before tumor cell challenge. Growth of tumors was monitored twice a week by inspection and palpation.

### R. Statistical Analysis

Data expressed as mean±standard deviations (SD) are representative of at least two different experiments. Comparisons between individual data points were made by 2-tailed Student's t test. A P value of less than 0.05 was considered significant.

### Example 2

### Vaccination with HPV-16 Pseudovirions Containing OVA DNA Elicits Strong OVA-Specific CD8+ T Cell Immune Responses in a Dose-Dependent Manner

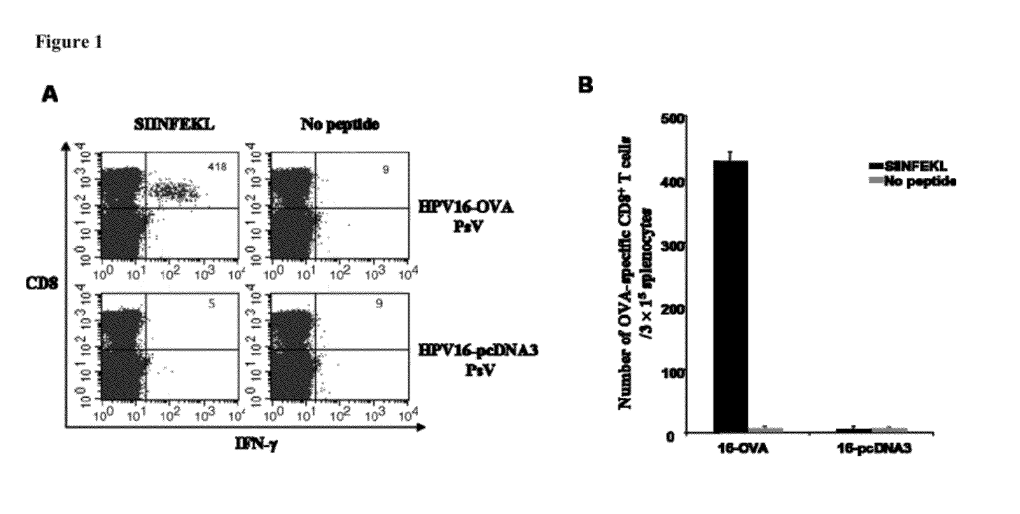

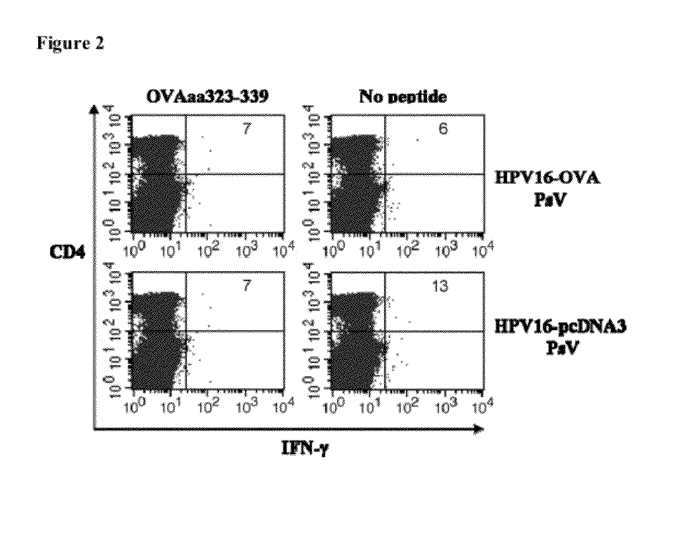

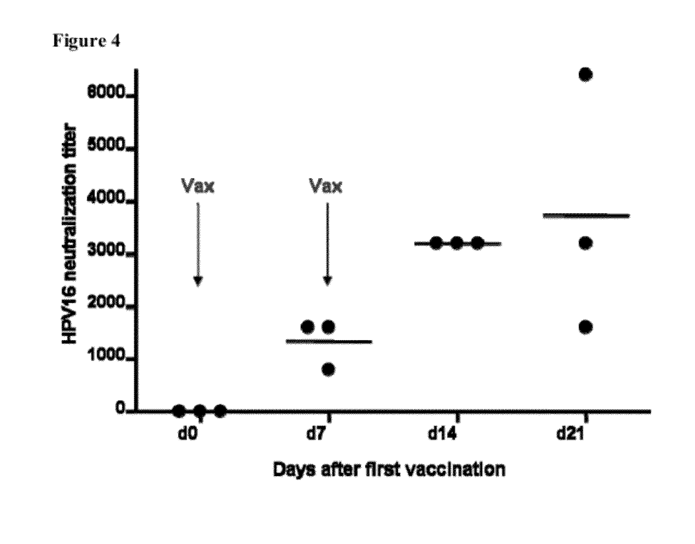

In order to determine whether OVA-specific CD8+ T cell immune responses are generated by vaccination with HPV-16 pseudovirions containing OVA DNA (HPV16-OVA pseudovirions), C57BL/6 mice (5 per group) were vaccinated with HPV 16-OVA pseudovirions or HPV16-pcDNA3 pseudovirions at a dose of 5 μg L1 protein/mouse via subcutaneous injection. All mice were boosted 7 days later with the same regimen. One week after last vaccination, splenocytes were prepared and stimulated with OVA peptide and then analyzed for OVA-specific CD8⁺ T cells by intracellular cytokine staining followed by flow cytometry analysis. As shown in FIGS. 1A and 1B, mice vaccinated with HPV 16-OVA pseudovirions generated significantly higher number of OVA-specific CD8+ T cell immune responses compared to mice vaccinated with the control HPV16-pcDNA3 pseudovirions. Significant OVA-specific CD4+ T cell immune responses in mice vaccinated with HPV 16-OVA pseudovirions or HPV16-pcDNA3 pseudovirions were note detected (FIG. 2). The OVA-specific antibody responses in mice vaccinated with HPV 16-OVA pseudovirions over time were also investigated. It was found that mice vaccinated with HPV 16-OVA pseudovirions did not generate detectable levels of OVA-specific antibody responses (FIG. 3). Thus, the data indicate that subcutaneous vaccination with HPV-16-OVA pseudovirions effectively presents OVA via MHC class I to generate significant OVA-specific CD8+ T cell immune responses. In addition, the serum titer of HPV-16 neutralizing antibodies in vaccinated mice was also checked. It was found that the HPV16 neutralizing antibodies could be detected 7 days after the initial vaccination and was significantly elevated 2 weeks after the initial vaccination (FIG. 4).

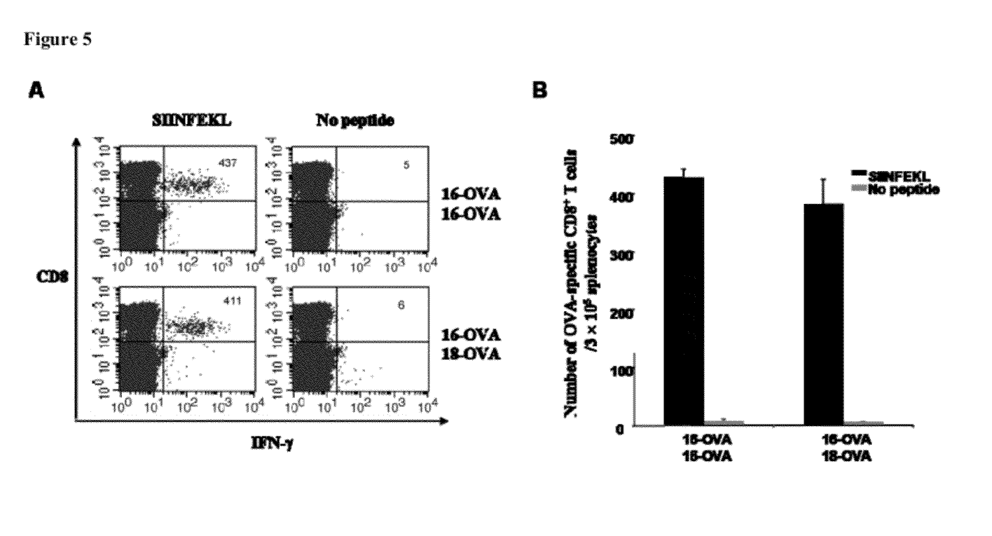

It was hypothesized that the induction of HPV-specific neutralizing antibodies by the priming dose of pseudovirions could limit the potency of the subsequent booster dose. It was further hypothesized that one way to eliminate this concern would be by boosting with pseudovirion derived from a different HPV type, since HPV neutralizing antibodies are primarily type-restricted. Therefore, the OVA-specific CD8+ T cell immune responses generated by prime-boost vaccination with the same type of pseudovirions (homologous vaccination) was compared against such responses with prime-boost vaccination with different types of pseudovirions (heterologous vaccination). C57BL/6 mice (5 per group) were vaccinated with HPV16-OVA pseudovirions via subcutaneous (footpad) injection. 7 days later, one group was boosted with HPV 16-OVA pseudovirions (homologous vaccination), and another group was boosted with HPV 18-OVA pseudovirions (heterologous vaccination). One week after last vaccination, splenocytes from vaccinated mice were isolated and analyzed for OVA-specific CD8⁺ T cells by intracellular cytokine staining followed by flow cytometry analysis. As shown in FIGS. 5A and 5B, mice vaccinated with HPV-16-OVA pseudovirions by homologous vaccination generated similar number of OVA-specific CD8+ T cell immune responses compared to mice vaccinated by heterologous vaccination. Thus, the data indicate that homologous vaccination with HPV-16-OVA pseudovirions generates comparable OVA-specific CD8+ T cell immune responses compared to heterologous vaccination with different type of HPV pseudovirions when performed one week apart.

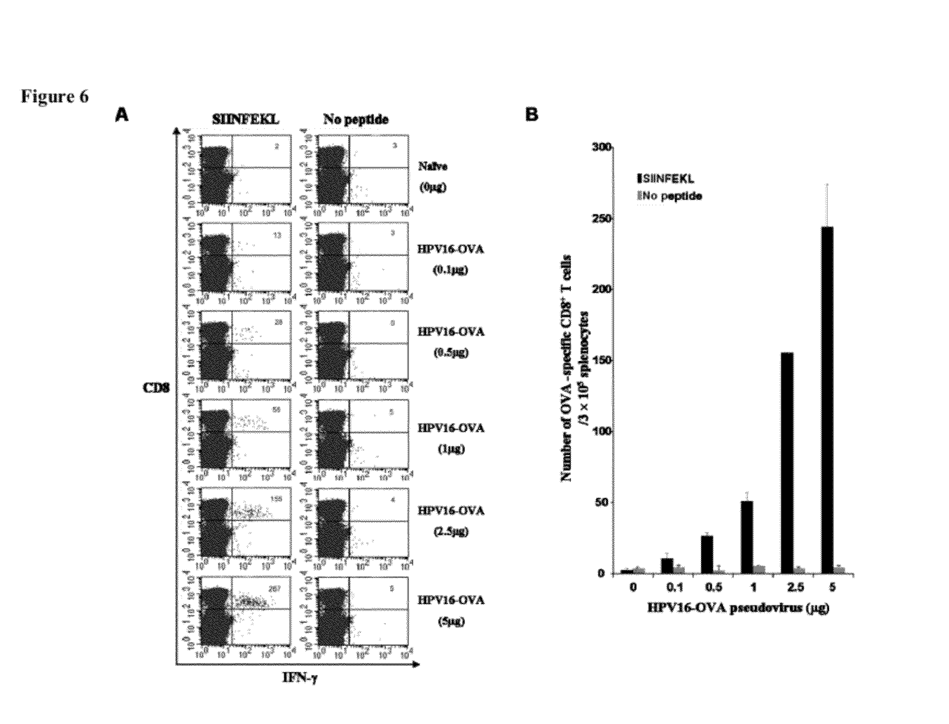

In order to determine the dose response of OVA-specific CD8+ T cell immune responses induced by vaccination with HPV 16-OVA pseudovirions, C57BL/6 mice (5 per group) were vaccinated with increasing doses of HPV 16-OVA pseudovirions (0.1, 0.5, 1, 2.5, 5 μg) via subcutaneous injection. All mice were boosted 7 days later with the same regimen. One week after last vaccination, splenocytes from vaccinated mice were isolated and analyzed for OVA-specific CD8⁺ T cells by intracellular cytokine staining followed by flow cytometry analysis. As shown in FIGS. 6A and 6B, mice vaccinated with the highest dose of HPV-16-OVA pseudovirions generated the highest number of OVA-specific CD8+ T cell immune responses. Thus, the data indicate that the level of OVA-specific CD8+ T cell immune responses increased with increasing dose of HPV 16-OVA pseudovirion vaccination.

### Example 3

### The Infectivity Mediated by the L2 Minor Capsid Protein on the HPV16-OVA Pseudovirion is Essential for the Generation of Antigen-Specific CD8+ T Cell Responses in Vaccinated Mice

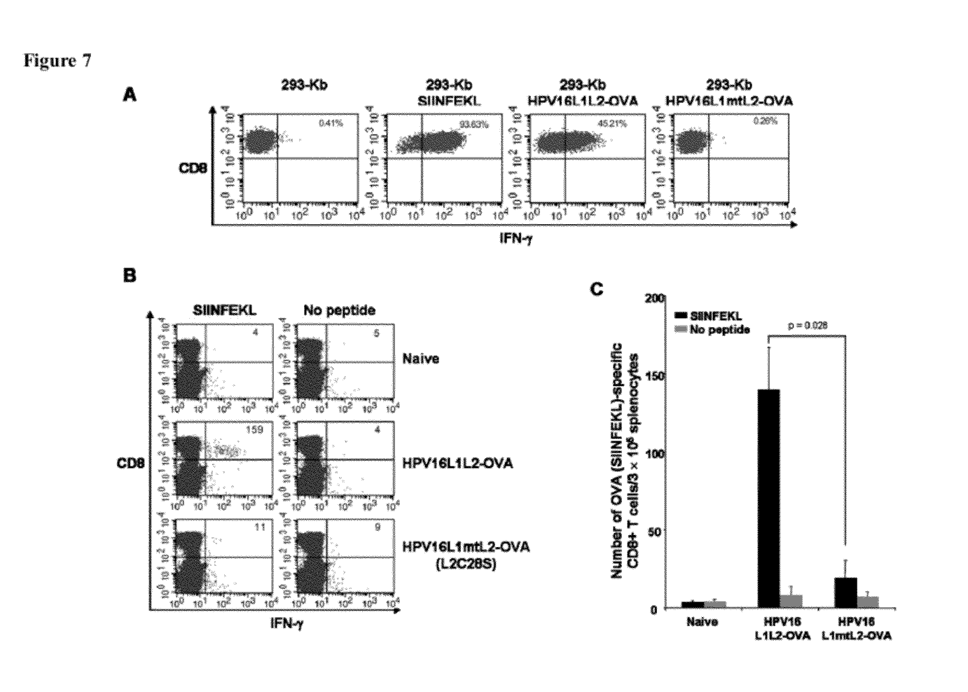

L2 minor capsid protein has been shown to be crucial for the infection of cells by papillomavirus pseudovirions (Campos et al., PLoS ONE, 4:e4463 (2009); Gambhira et al. Virol. J, 6:176 (2009)). In order to determine if infection mediated by L2 plays an essential role in the generation of antigen-specific CD8+ T cell immune responses in mice vaccinated with HPV16 pseudovirions, HPV 16-OVA pseudovirions were generated having a single amino acid mutation (amino acid 28 from Cysteine to Serine) in the L2 protein of the pseudovirion (HPV16L1mtL2-OVA pseudovirion), which abolishes the infectivity of pseudovirions (Gambhira et al. Virol. J, 6:176 (2009)). 293-Kb cells were infected with HPV16L1L2-OVA or the mutant HPV16L1mtL2-OVA pseudovirus, incubated with OVA-specific CD8+ T cells and then analyzed by intracellular IFN-γ staining. As shown in FIG. 7A, 293-Kb cells infected with L2 mutated HPV16-OVA pseudovirus demonstrated significant reduction in their ability to activate OVA-specific CD8+ T cells compared to cells infected with wild-type HPV 16-OVA pseudovirus. The data indicate that an intact L2 is essential for infection of 293-Kb cells by pseudovirion to lead to MHC class I presentation of OVA antigen.

In order to determine whether the intact L2 in the pseudovirions is essential for the generation of antigen-specific CD8+ T cell immune responses in vaccinated mice, C57BL/6 mice (5 per group) were vaccinated with HPV 16-OVA pseudovirions or the mutant HPV16L1mtL2-OVA pseudovirions via footpad injection. All mice were boosted 7 days later with the same regimen. One week after last vaccination, splenocytes were prepared and stimulated with OVA peptide and analyzed for OVA-specific CD8⁺ T cells by intracellular cytokine staining followed by flow cytometry analysis. As shown in FIGS. 7B and 7C, mice vaccinated with the mutant HPV16L1mtL2-OVA pseudovirions generated significantly decreased number of OVA-specific CD8+ T cell immune responses compared to mice vaccinated with the wild type HPV-16L1L2-OVA pseudovirions. Taken together, the data indicate that the infectivity of the HPV pseudovirions mediated by the intact L2 is essential for their ability to generate antigen-specific CD8+ T cell immune responses in vaccinated mice.

### Example 4

### Vaccination with HPV-16 Pseudovirions Containing OVA DNA Leads to Strong Protective Antitumor Effects Against Ova-Expressing Tumors in Vaccinated Mice

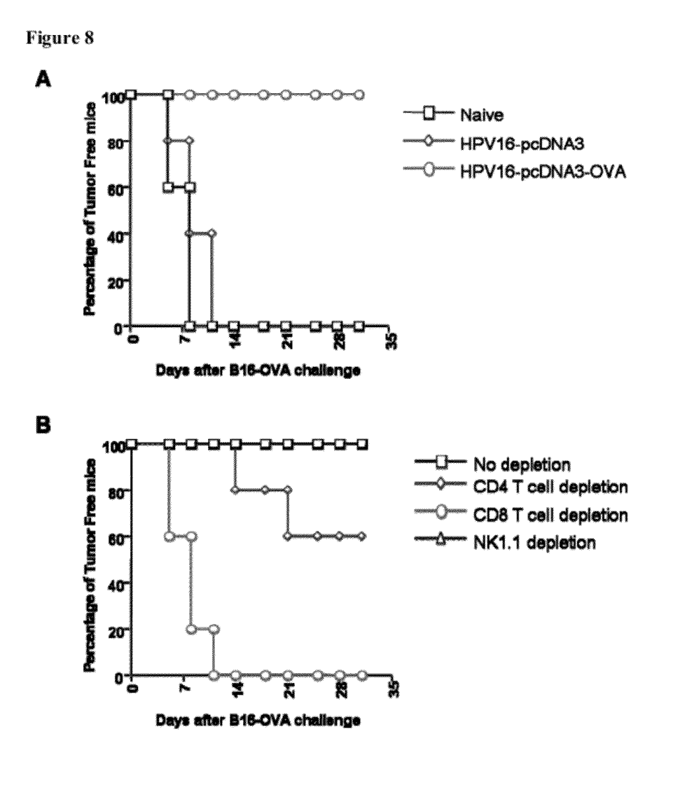

In order to assess the cytotoxic activity of OVA-specific CD8+ T cell immune responses generated by vaccination with HPV 16-OVA pseudovirions, C57BL/6 mice (5 per group) were vaccinated with HPV 16-OVA or HPV16-pcDNA3 via footpad injection. The mice were boosted twice with the same regimen at day 7 and day 14. One week after last vaccination, the mice were injected with B16-OVA cells subcutaneously. Tumor growth was monitored twice a week. As shown in FIG. 8A, mice vaccinated with HPV 16-OVA pseudovirions demonstrated significantly higher percentage of tumor-free mice compared to mice vaccinated with HPV16-pcDNA3 pseudovirions. For antibody depletion of specific immune cell subsets, the mice were treated with antibodies against mouse CD4, CD8 and NK1.1 at the same time of last vaccination via intraperitoneal injection. Depletion of CD8+ T cells in mice vaccinated with HPV 16-OVA pseudovirions significantly lowered the percentage of tumor-free mice compared to vaccinated mice with CD4 or NK1.1 depletion or no depletion (FIG. 8B). Thus, the data indicate that vaccination with HPV-16 pseudovirions containing OVA DNA leads to strong protective antitumor effects against B16-OVA tumors in vaccinated mice and that CD8+ T cells play a major role in the antitumor effects.

### Example 5

### Vaccination with HPV16-OVA Pseudovirions Elicits Significantly Stronger OVA-Specific CD8+ T Cell Immune Responses Compared to Intradermal Vaccination with Naked OVA DNA

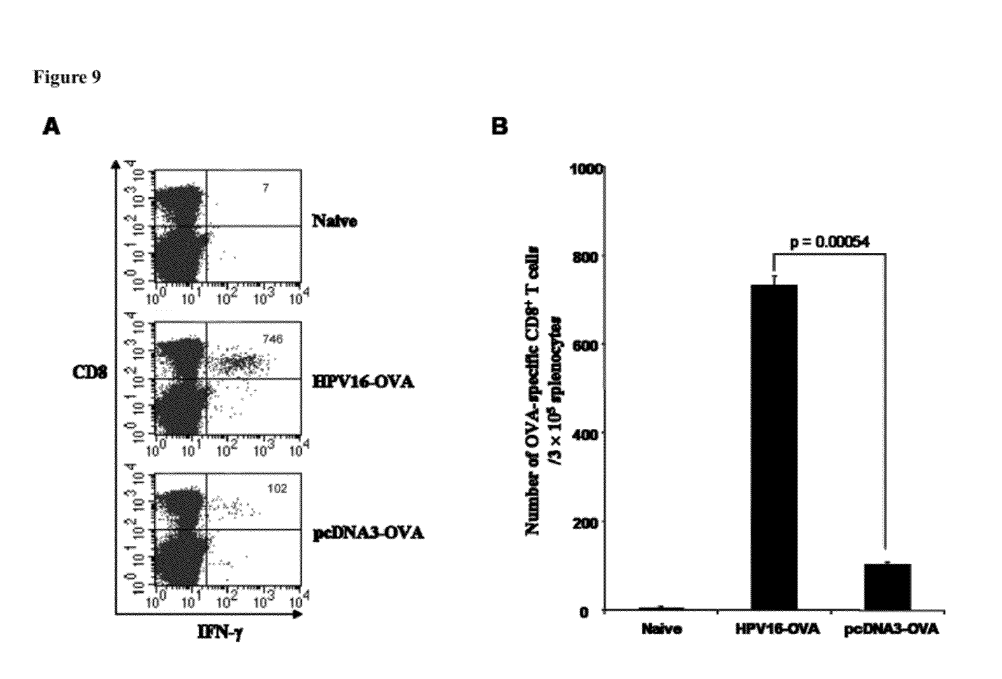

Intradermal vaccination with naked DNA via needles or gene gun routes of administration are used to generate potent antigen-specific immune responses by naked DNA vaccines in preclinical and clinical studies (Trimble et al., Vaccine, 21:4036-4042 (2003); Gurunathan et al., Annu. Rev. Immunol., 18:927-974 (2000)). In order to compare the OVA-specific immune responses generated by HPV16-OVA pseudovirion vaccination with intradermal vaccination with naked OVA DNA, C57BL/6 mice (5 per group) were vaccinated with HPV16-OVA pseudovirions via subcutaneous injection or with pcDNA3-OVA DNA via gene gun. All mice were boosted 7 days later with the same dose and regimen. One week after last vaccination, splenocytes from vaccinated mice were isolated and analyzed for OVA-specific CD8⁺ T cells by intracellular cytokine staining followed by flow cytometry analysis. As shown in FIGS. 9A and 9B, mice vaccinated with HPV16-OVA pseudovirions generated significantly higher number of OVA-specific CD8+ T cell immune responses compared to mice vaccinated with naked OVA DNA vaccination. Thus, the data indicate that vaccination with HPV 16-OVA pseudovirions generates a significantly higher number of OVA-specific CD8+ T cell immune responses than vaccination with naked OVA DNA.

### Example 6

### HPV Pseudovirions can Efficiently Infect Bone Marrow Derived Dendritic Cells In Vitro and can be Taken Up by CD11c+ and B220+ Cells in the Draining Lymph Nodes of Vaccinated Mice

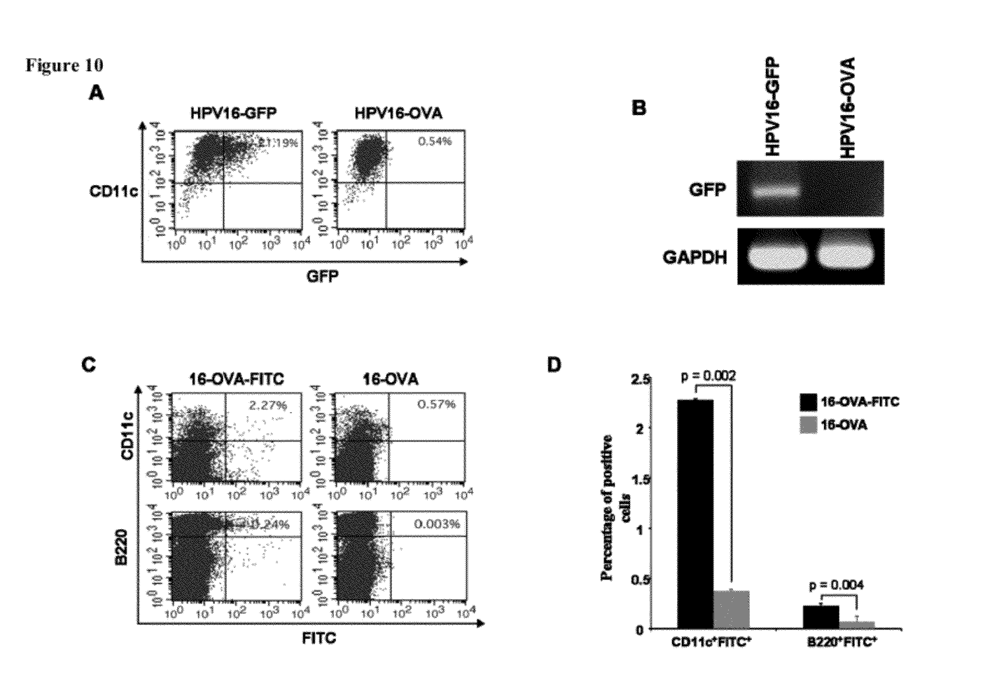

In order to determine whether HPV pseudovirions can infect bone marrow derived dendritic cells (BMDC), BMDCs were cultured in the presence of GM-CSF for 4 days and HPV16 pseudovirions containing DNA encoding GFP or OVA were added to the culture. After 72 hours, BMDCs were harvested and GFP expression was examined by flow cytometry analysis. As shown in FIG. 10A, a significant percentage of CD11c+ bone marrow-derived dendritic cells infected with pseudovirions containing GFP DNA, but not OVA DNA, demonstrated GFP expression.

In order to determine whether mice vaccinated with HPV16 pseudovirions containing GFP leads to the expression of GFP in the draining lymph nodes, C57BL/6 mice (5 per group) were vaccinated with HPV16 pseudovirions carrying GFP or OVA DNA via footpad injection. After 72 hours, draining lymph nodes were harvested, total RNA was isolated and RT-PCR was performed to detect GFP mRNA expression. As shown in FIG. 10B, mice vaccinated with HPV16 pseudovirions carrying GFP DNA, but not pseudovirions carrying OVA DNA, demonstrated detectable expression of GFP in draining lymph nodes.

In order to further determine the type of cells that can carry HPV 16-OVA pseudovirions into draining lymph nodes, HPV16-OVA pseudovirions were conjugated with FITC and the labeled pseudovirions were injected into C57BL/6 mice via subcutaneous injection. The draining lymph nodes of the injected mice were harvested after 48 hours and the presence of FITC-labeled pseudovirions within the cells in the draining lymph nodes was analyzed by flow cytometry. As shown in FIGS. 10C and 10D, the B220+ cells and CD11c+ cells in draining lymph nodes comprised a significant percentage of the FITC+ cells (2.27% CD11c+ cells and 0.24% B220+ cells) indicating uptake of the HPV 16-OVA pseudovirions. Thus, the data indicate that dendritic cells in draining lymph nodes can significantly uptake FITC-labeled HPV 16-OVA pseudovirions and a subset of B220+ cells in draining lymph nodes can uptake FITC-labeled HPV 16-OVA pseudovirions to a lesser extent.

Taken together, the data indicate that HPV pseudovirions can efficiently infect bone marrow derived dendritic cells in vitro. Furthermore, administration of HPV pseudovirions in vivo can lead to the uptake of pseudovirions by CD11c+ cells and B220+ cells in draining lymph nodes, resulting in the expression of the encoded protein.

### Example 7

### Treatment of HPV16 Pseudovirions with Furin Leads to Enhanced Pseudovirion Infection and Improved Antigen Presentation in Infected Cells

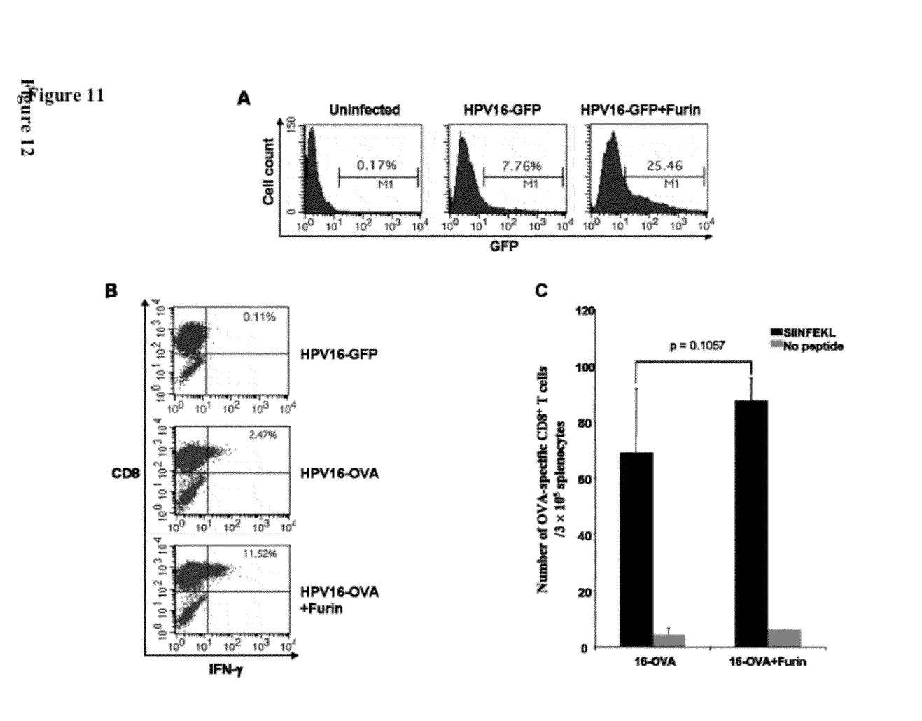

Several previous studies have implicated furin in the process of papillomavirus infection (Gambhira et al. Virol. J, 6:176 (2009); Kines et al., Proc. Natl. Acad. Sci. USA, 106:20458-20463 (2009); Day et al., J. Virol., 82:4638-4646 (2008); Day et al., J. Virol., 82:12565-12568 (2008)). It was recently found that infectious entry of papillomaviruses is dependent upon the cleavage of the L2 protein by furin (Day et al., Future Microbiol., 4:1255-1262 (2009)). Thus, in order to determine whether HPV16 pseudovirion infection can be enhanced by pretreatment with furin, DC-1 cells were infected with HPV16-GFP pseudovirions with or without pretreatment with furin. The infection of DC-1 cells by HPV16-GFP pseudovirions was analyzed by characterization of GFP expression in DC-1 cells using flow cytometry. As shown in FIG. 11A, DC-1 cells infected with HPV16-GFP pseudovirions in the presence of furin demonstrated significantly higher percentage of GFP+ cells compared to DC-1 cells infected with HPV16-GFP pseudovirions without furin. Thus, the data indicate that treatment of HPV 16 pseudovirions with furin leads to enhanced pseudovirion infection.

In order to determine whether the enhanced pseudovirion infection translated into improved antigen presentation in the infected cells, DC-1 cells were infected with HPV16-OVA pseudovirions with or without the treatment with furin. The infected cells were collected 72 hours after infection, and co-cultured with OVA-specific CD8+OT-1 T cells (E:T ratio at 1:1) overnight. Activation of OT-1 T cells was analyzed by IFN-γ intracellular staining followed by flow cytometry analysis. As shown in FIG. 11B, cells infected with HPV 16-OVA pseudovirions in the presence of furin demonstrated significantly higher percentage of activated IFNγ-secreting CD8+ T cells compared to cells infected HPV16-OVA pseudovirions without furin. This indicates that treatment of HPV16 pseudovirions with furin leads to enhanced antigen presentation in the infected cells. Thus, the data suggest that treatment of HPV16 pseudovirions with furin leads to enhanced pseudovirion infection of DC-1 cells, resulting in improved antigen presentation in infected cells.

In order to determine whether furin pretreatment enhances antigen presentation, producing a stronger immune response, C57BL/6 mice were vaccinated with HPV16-OVA pseudovirions with or without furin treatment. All mice were boosted 7 days later with the same dose and regimen. One week after last vaccination, splenocytes were prepared and stimulated with OVA peptide and analyzed for OVA-specific CD8⁺ T cells by intracellular cytokine staining followed by flow cytometry analysis. As shown in FIG. 11C, the difference in the OVA-specific CD8+ T cell immune responses generated in mice vaccinated with HPV16-OVA pseudovirions treated with furin compared to mice vaccinated with HPV16-OVA pseudovirions without furin treatment was not statistically significant (p=0.1057).

Taken together, although treatment of HPV16 pseudovirions with furin led to enhanced pseudovirion infection and improved antigen presentation in DC-1 cells, it does not significantly increase the OVA-specific CD8+ T cell immune responses in vaccinated mice.

### Example 8

### Skin-Tropic HPV-2 Pseudovirions Harboring Naked Exogenous DNA Effectively Infects Mouse and Human Skin Cells

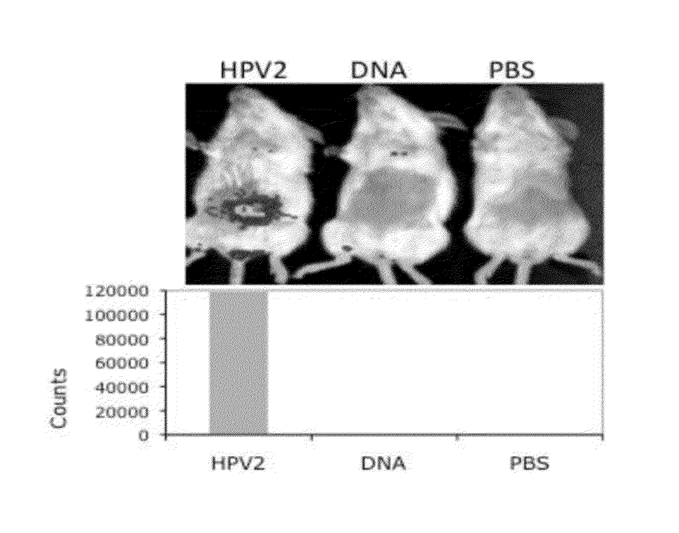

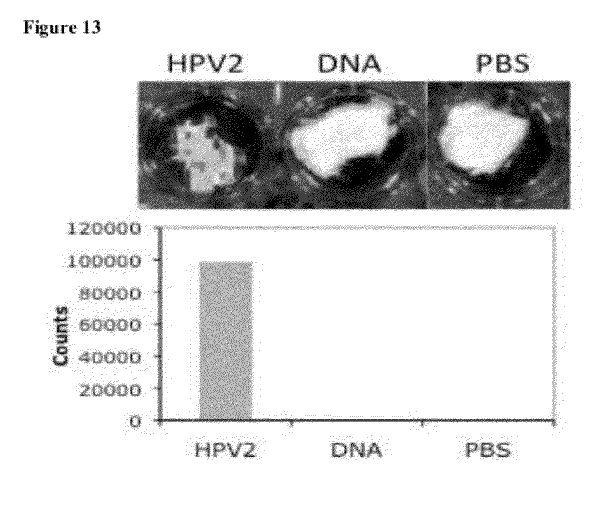

Skin of mice were infected in vivo with skin-tropic HPV-2 pseudovirions expressing luciferase (HPV-2/luc psV). The expression of luciferase was characterized using non-invasive luminescence imaging. As shown in FIG. 12, mice infected with HPV-2/luc psV showed significant expression of luciferase in the skin. By contrast, mice infected with an equivalent amount of luciferase DNA or PBS did not show detectable luciferase expression. Thus, the data indicate that HPV-2 pseudovirions are capable of infecting the skin of mice and of delivering naked DNA much more efficiently than delivery of naked DNA without pseudovirions. Similar results have also been demonstrated with HPV-2/luc psV infection of human skin grafts in vitro (FIG. 13).

LISTING OF ADDITIONAL SEQUENCES

| | |
|---|---|
| SEQ ID NO: 1 (coded protein disclosed as SEQ ID NO: 2) | |
| atg cat gga gat aca cct aca ttg cat gaa tat atg tta gat ttg caa cca gag aca act | 60 |
| Met His Gly Asp Thr Pro Thr Leu His Glu Tyr Met Leu Asp Leu Gln Pro Glu Thr Thr | 20 |
| gat ctc tac tgt tat gag caa tta aat gac agc tca gag gag gag gat gaa ata gat ggt | 120 |
| Asp Leu Tyr Cys Tyr Glu Gln Leu Asn Asp Ser Ser Glu Glu Glu Asp Glu Ile Asp Gly | 40 |
| cca gct gga caa gca gaa ccg gac aga gcc cat tac aat att gta acc ttt tgt tgc aag | 180 |
| Pro Ala Gly Gln Ala Glu Pro Asp Arg Ala His Tyr Asn Ile Val Thr Phe Cys Cys Lys | 60 |
| tgt gac tct acg ctt cgg ttg tgc gta caa agc aca cac gta gac att cgt act ttg gaa | 240 |
| Cys Asp Ser Thr Leu Arg Leu Cys Val Gln Ser Thr His Val Asp Ile Arg Thr Leu Glu | 80 |
| gac ctg tta atg ggc aca cta gga att gtg tgc ccc atc tgt tct cag gat aag ctt | 297 |
| Asp Leu Leu Met Gly Thr Leu Gly Ile Val Cys Pro Ile Cys Ser Gln Asp Lys Leu | 99 |
| SEQ ID NO: 2 | |
| MHGDTPTLHE YMLDLQPETT DLYCYEQLND SSEEEDEIDG PAGQAEPDRA HYNIVTFCCK CDSTLRLCVQ STHVDIRTLE | |
| DLLMGTLGIV CPICSQDKL | 99 |
| SEQ ID NO: 3 | |
| MHGDTPTLHE YMLDLQPETT DLYGYEGLND SSEEEDEIDG PAGQAEPDRA HYNIVTFCCK CDSTLRLCVQ STHVDIRTLE | |
| DLLMGTLGIV CPICSQKP | 97 |
| SEQ ID NO: 4 (coded protein disclosed as SEQ ID NO: 5) | |
| atg cac caa aag aga act gca atg ttt cag gac cca cag gag cga ccc aga aag tta cca | 60 |
| Met His Gln Lys Arg Thr Ala Met Phe Gln Asp Pro Gln Glu Arg Pro Arg Lys Leu Pro | 20 |
| cag tta tgc aca gag ctg caa aca act ata cat gat ata ata tta gaa tgt gtg tac tgc | 120 |
| Gln Leu Cys Thr Glu Leu Gln Thr Thr Ile His Asp Ile Ile Leu Glu Cys Val Tyr Cys | 40 |
| aag caa cag tta ctg cga cgt gag gta tat gac ttt gct ttt cgg gat tta tgc ata gta | 180 |
| Lys Gln Gln Leu Leu Arg Arg Glu Val Tyr Asp Phe Ala Phe Arg Asp Leu Cys Ile Val | 60 |
| tat aga gat ggg aat cca tat gct gta tgt gat aaa tgt tta aag ttt tat tct aaa att | 240 |
| Tyr Arg Asp Gly Asn Pro Tyr Ala Val Cys Asp Lys Cys Leu Lys Phe Tyr Ser Lys Ile | 80 |
| agt gag tat aga cat tat tgt tat agt ttg tat gga aca aca tta gaa cag caa tac aac | 300 |
| Ser Glu Tyr Arg His Tyr Cys Tyr Ser Leu Tyr Gly Thr Thr Leu Glu Gln Gln Tyr Asn | 100 |
| aaa ccg ttg tgt gat ttg tta att agg tgt att aac tgt caa aag cca ctg tgt cct gaa | 360 |
| Lys Pro Leu Cys Asp Leu Leu Ile Arg Cys Ile Asn Cys Gln Lys Pro Leu Cys Pro Glu | 120 |
| gaa aag caa aga cat ctg gac aaa aag caa aga ttc cat aat ata agg ggt cgg tgg acc | 420 |
| Glu Lys Gln Arg His Leu Asp Lys Lys Gln Arg Phe His Asn Ile Arg Gly Arg Trp Thr | 140 |
| ggt cga tgt atg tct tgt tgc aga tca tca aga aca cgt aga gaa acc cag ctg taa | 474 |
| Gly Arg Cys Met Ser Cys Cys Arg Ser Ser Arg Thr Arg Arg Glu Thr Gln Leu stop | 158 |
| SEQ ID NO: 5 | |
| MHQKRTAMFQ DPQERPRKLP QLCTELQTTI HDIILECVYC KQQLLRREVY DFAFRDLCIV YRDGNPYAVC DKCLKFYSKI | |
| SEYRHYCYSL YGTTLEQQYN KPLCDLLIRC INCQKPLCPE EKQRHLDKKQ RFHNIRGRWT GRCMSCCRSS RTRRETQL | 158 |
| SEQ ID NO: 6 | |
| MFQDPQERPR KLPQLCTELQ TTIHDIILEC VYCKQQLLRR EVYDFAFRDL CIVYRDGNPY AVCDKCLKFY SKISEYRHYC | |
| YSLYGTTLEQ QYNKPLCDLL IRCINCQKPL CPEEKQRHLD KKQRFHNIRG RWTGRCMSCC RSSRTRRETQ L | |

SEQ ID NO: 7
atgaaggcaaacctactggtcctgttaagtgcacttgcagctgcagatgcagacacaatatgtataggctaccatgcgaacaattcaaccga
cactgttgacacagtactcgagaagaatgtgacagtgacacactctgttaacctgctcgaagacagccacaacggaaaactatgtagattaa
aaggaatagccccactacaattggggaaatgtaacatcgccggatggctcttgggaaacccagaatgcgacccactgcttccagtgagatca
tggtcctacattgtagaaacaccaaactctgagaatggaatatgttatccaggagatttcatcgactatgaggagctgagggagcaattgag
ctcagtgtcatcattcgaaagattcgaaatatttcccaaagaaagctcatggcccaaccacaacacaaacggagtaacggcagcatgctccc
atgaggggaaaagcagtttttacagaaatttgctatggctgacggagaaggagggctcatacccaaagctgaaaaattcttatgtgaacaaa
aaagggaaagaagtccttgtactgtggggtattcatcacccgcctaacagtaaggaacaacagaatatctatcagaatgaaaatgcttatgt
ctctgtagtgacttcaaattataacaggagatttaccccggaaatagcagaaagacccaaagtaagagatcaagctgggaggatgaactatt
actggaccttgctaaaacccggagacacaataatatttgaggcaaatggaaatctaatagcaccaatgtatgctttcgcactgagtagaggc
tttgggtccggcatcatcacctcaaacgcatcaatgcatgagtgtaacacgaagtgtcaaacacccctgggagctataaacagcagtctccc
ttaccagaatatacacccagtcacaataggagagtgcccaaaatacgtcaggagtgccaaattgaggatggttacaggactaaggaacactc
cgtccattcaatccagaggtctatttggagccattgccggttttattgaagggggatggactggaatgatagatggatggtatggttatcat
catcagaatgaacagggatcaggctatgcagcggatcaaaaaagcacacaaaatgccattaacgggattacaaacaaggtgaacactgttat
cgagaaaatgaacattcaattcacagctgtgggtaaagaattcaacaaattagaaaaaaggatggaaaatttaaataaaaaagttgatgatg
gatttctggacatttggacatataatgcagaattgttagttctactggaaaatgaaaggactctggatttccatgactcaaatgtgaagaat
ctgtatgagaaagtaaaaagccaattaaagaataatgccaaagaaatcggaaatggatgttttgagttctaccacaagtgtgacaatgaatg
catggaaagtgtaagaaatgggacttatgattatcccaaatattcagaagagtcaaagttgaacagggaaaaggtagatggagtgaaattgg
aatcaatggggatctatcagattctggcgatctactcaactgtcgccagttcactggtgcttttggtctccctgggggcaatcagtttctgg
atgtgttctaatggatctttgcagtgcagaatatgcatctga
SEQ ID NO: 8
MKANLLVLLS ALAAADADTI CIGYHANNST DTVDTVLEKN VTVTHSVNLL EDSHNGKLCR LKGIAPLQLG KCNIAGWLLG
NPECDPLLPV RSWSYIVETP NSENGICYPG DFIDYEELRE QLSSVSSFER FEIFPKESSW PNHNTNGVTA ACSHEGKSSF
YANLLWLTEK EGSYPKLKNS YVNKKGKEVL VLWGIHHPPN SKEQQNIYQN ENAYVSVVTS NYNRRFTPEI AERPKVRDQA
GRMNYYWTLL KPGDTIIFEA NGNLIAPMYA FALSAGFGSG IITSNASMHE CNTKCQTPLG AINSSLPYQN IHPVTIGECP
KYVASAKLRM VTGLRNTPSI QSRGLFGAIA GFIEGGWTGM IDGWYGYHHQ NEQGSGYAAD QKSTQNAING ITNKVNTVIE
KMNIQFTAVG KEFNKLEKRM ENLNKKVDDG FLDIWTYNAE LLVLLENERT LDFHDSNVKN LYEKVKSQLK NNAKEIGNGC
FEFYHKCDNE CMESVRNGTY DYPKYSEESK LNREKVDGVK LESMGIYQIL AIYSTVASSL VLLVSLGAIS FWMCSNGSLQ
CRICI
SEQ ID NO: 9
MGSIGAASMEFCFDVFKELKVHHANENIFYCPIAIMSALAMVYLGAKDSTRTQINKVVRFDKLPGFGDSIEAQCGTSVNV
HSSLRDILNQITKPNDVYSFSLASRLYAEERYPILPEYLQCVKELYRGGLEPINFQTAADQARELINSWVESQTNGIIRN
VLQPSSVDSQTAMVLVNAIVFKGLWEKTFKDEDTQAMPFRVTEQESKPVQMMYQIGLFRVASMASEKMKILELPFASGTM
SMLVLLPDEVSGLEQLESIINFEKLTEWTSSNVMEERKIKVYLPRMKMEEKYNLTSVLMAMGITDVFSSSANLSGISSAE
SLKISQAVHAAHAEINEAGREVVGSAEAGVDAASVSEEFRADHPFLFCIKHIATNAVLFFGRCVSP
SEQ ID NO: 10
ATGGCGGCCCCCGGCGCCCGGCGGCCGCTGCTCCTGCTGCTGCTGGCAGGCCTTGCACATGGCGCCTCAGCACTCTTTGAGGATCTAATCAT
GCATGGAGATACACCTACATTGCATGAATATATGTTAGATTTGCAACCAGAGACAACTGATCTCTACTGTTATGAGCAATTAAATGACAGCT
CAGAGGAGGAGGATGAAATAGATGGTCCAGCTGGACAAGCAGAACCGGACAGAGCCCATTACAATATTGTTACCTTTTGTTGCAAGTGTGAC
TCTACGCTTCGGTTGTGCGTACAAAGCACACACGTAGACATTCGTACTTTGGAAGACCTGTTAATGGGCACACTAGGAATTGTGTGCCCCAT
CTGTTCTCAGGATCTTAACAACATGTTGATCCCCATTGCTGTGGGCGGTGCCCTGGCAGGGCTGGTCCTCATCGTCCTCATTGCCTACCTCA
TTGGCAGGAAGAGGAGTCACGCCGGCTATCAGACCATCTAG
SEQ ID NO: 11
MAAPGARRPL LLLLLAGLAH GASALFEDLI MHGDTPTLHE YMLDLQPETT DLYCYEQLND SSEEEDEIDG PAGQAEPDRA
HYNIVTFCCK CDSTLRLCVQ STHVDIRTLE DLLMGTLGIV CPICSQDLNN MLIPIAVGGA LAGLVLIVLI AYLIGRKRSH
AGYQTI
SEQ ID NO: 12
GACGGATCGGGAGATCTCCCGATCCCCTATGGTCGACTCTCAGTACAATCTGCTCTGATGCCGCATAGTTAAGCCAGTATCTGCTCCCTGCT
TGTGTGTTGGAGGTCGCTGAGTAGTGCGCGAGCAAAATTTAAGCTACAACAAGGCAAGGCTTGACCGACAATTGCATGAAGAATCTGCTTAG
GGTTAGGCGTTTTGCGCTGCTTCGCGATGTACGGGCCAGATATACGCGTTGACATTGATTATTGACTAGTTATTAATAGTAATCAATTACGG
GGTCATTAGTTCATAGCCCATATATGGAGTTCCGCGTTACATAACTTACGGTAAATGGCCCGCCTGGCTGACCGCCCAACGACCCCCGCCCA
TTGACGTCAATAATGACGTATGTTCCCATAGTAACGCCAATAGGGACTTTCCATTGACGTCAATGGGTGGACTATTTACGGTAAACTGCCCA
CTTGGCAGTACATCAAGTGTATCATATGCCAAGTACGCCCCCTATTGACGTCAATGACGGTAAATGGCCCGCCTGGCATTATGCCCAGTACA
TGACCTTATGGGACTTTCCTACTTGGCAGTACATCTACGTATTAGTCATCGCTATTACCATGGTGATGCGGTTTTGGCAGTACATCAATGGG
CGTGGATAGCGGTTTGACTCACGGGGATTTCCAAGTCTCCACCCCATTGACGTCAATGGGAGTTTGTTTTGGCACCAAAATCAACGGGACTT
TCCAAAATGTCGTAACAACTCCGCCCCATTGACGCAAATGGGCGGTAGGCGTGTACGGTGGGAGGTCTATATAAGCAGAGCTCTCTGGCTAA
CTAGAGAACCCACTGCTTACTGGCTTATCGAAATTAATACGACTCACTATAGGGAGACCCAAGCTGGCTAGCGTTTAAACGGGCCCTCTAGA
CTCGAGCGGCCGCCACTGTGCTGGATATCTGCAGAATTCatggcggcccccggcgcccggcggccgctgctcctgctgctgctggcaggcct
tgcacatggcgcctcagcactctttgaggatctaatcatgcatggagatacacctacattgcatgaatatatgttagatttgcaaccagaga
caactgatctctactgttatgagcaattaaatgacagctcagaggaggaggatgaaatagatggtccagctggacaagcagaaccggacaga
gcccattacaatattgttaccttttgttgcaagtgtgactctacgcttcggttgtgcgtacaaagcacacacgtagacattcgtactttgga
agacctgttaatgggcacactaggaattgtgtgccccatctgttctcaggatcttaacaacatgttgatccccattgctgtgggcggtgccc
tggcagggctggtcctcatcgtcctcattgcctacctcattggcaggaagaggagtcacgccggctatcagaccatctagGGATCCGAGCTC
GGTACCAAGCTTAAGTTTAAACCGCTGATCAGCCTCGACTGTGCCTTCTAGTTGCCAGCCATCTGTTGTTTGCCCCTCCCCCGTGCCTTCCT
TGACCCTGGAAGGTGCCACTCCCACTGTCCTTTCCTAATAAAATGAGGAAATTGCATCGCATTGTCTGAGTAGGTGTCATTCTATTCTGGGG
GGTGGGGTGGGGCAGGACAGCAAGGGGGAGGATTGGGAAGACAATAGCAGGCATGCTGGGGATGCGGTGGGCTCTATGGCTTCTGAGGCGGA
AAGAACCAGCTGGGGCTCTAGGGGGTATCCCCACGCGCCCTGTAGCGGCGCATTAAGCGCGGCGGGTGTGGTGGTTACGCGCAGCGTGACCG
CTACACTTGCCAGCGCCCTAGCGCCCGCTCCTTTCGCTTTCTTCCCTTCCTTTCTCGCCACGTTCGCCGGCTTTCCCCGTCAAGCTCTAAAT
CGGGGCATCCCTTTAGGGTTCCGATTTAGTGCTTTACGGCACCTCGACCCCAAAAAACTTGATTAGGGTGATGGTTCACGTAGTGGGCCATC
GCCCTGATAGACGGTTTTTCGCCCTTTGACGTTGGAGTCCACGTTCTTTAATAGTGGACTCTTGTTCCAAACTGGAACAACACTCAACCCTA
TCTCGGTCTATTCTTTTGATTTATAAGGGATTTTGGGGATTTCGGCCTATTGGTTAAAAAATGAGCTGATTTAACAAAAATTTAACGCGAAT
TAATTCTGTGGAATGTGTGTCAGTTAGGGTGTGGAAAGTCCCCAGGCTCCCCAGGCAGGCAGAAGTATGCAAAGCATGCATCTCAATTAGTC
AGCAACCAGGTGTGGAAAGTCCCCAGGCTCCCCAGCAGGCAGAAGTATGCAAAGCATGCATCTCAATTAGTCAGCAACCATAGTCCCGCCCC
TAACTCCGCCCATCCCGCCCCTAACTCCGCCCAGTTCCGCCCATTCTCCGCCCCATGGCTGACTAATTTTTTTTATTTATGCAGAGGCCGAG
GCCGCCTCTGCCTCTGAGCTATTCCAGAAGTAGTGAGGAGGCTTTTTTGGAGGCCTAGGCTTTTGCAAAAAGCTCCCGGGAGCTTGTATATC
CATTTTCGGATCTGATCAAGAGACAGGATGAGGATCGTTTCGCATGATTGAACAAGATGGATTGCACGCAGGTTCTCCGGCCGCTTGGGTGG
AGAGGCTATTCGGCTATGACTGGGCACAACAGACAATCGGCTGCTCTGATGCCGCCGTGTTCCGGCTGTCAGCGCAGGGGCGCCCGGTTCTT
TTTGTCAAGACCGACCTGTCCGGTGCCCTGAATGAACTGCAGGACGAGGCAGCGCGGCTATCGTGGCTGGCCACGACGGGCGTTCCTTGCGC
AGCTGTGCTCGACGTTGTCACTGAAGCGGGAAGGGACTGGCTGCTATTGGGCGAAGTGCCGGGGCAGGATCTCCTGTCATCTCACCTTGCTC
CTGCCGAGAAAGTATCCATCATGGCTGATGCAATGCGGCGGCTGCATACGCTTGATCCGGCTACCTGCCCATTCGACCACCAAGCGAAACAT
CGCATCGAGCGAGCACGTACTCGGATGGAAGCCGGTCTTGTCGATCAGGATGATCTGGACGAAGAGCATCAGGGGCTCGCGCCAGCCGAACT
GTTCGCCAGGCTCAAGGCGCGCATGCCCGACGGCGAGGATCTCGTCGTGACCCATGGCGATGCCTGCTTGCCGAATATCATGGTGGAAAATG
GCCGCTTTTCTGGATTCATCGACTGTGGCCGGCTGGGTGTGGCGGACCGCTATCAGGACATAGCGTTGGCTACCCGTGATATTGCTGAAGAG
CTTGGCGGCGAATGGGCTGACCGCTTCCTCGTGCTTTACGGTATCGCCGCTCCCGATTCGCAGCGCATCGCCTTCTATCGCCTTCTTGACGA
GTTCTTCTGAGCGGGACTCTGGGGTTCGAAATGACCGACCAAGCGACGCCCAACCTGCCATCACGAGATTTCGATTCCACCGCCGCCTTCTA
TGAAAGGTTGGGCTTCGGAATCGTTTTCCGGGACGCCGGCTGGATGATCCTCCAGCGCGGGGATCTCATGCTGGAGTTCTTCGCCCACCCCA
ACTTGTTTATTGCAGCTTATAATGGTTACAAATAAAGCAATAGCATCACAAATTTCACAAATAAAGCATTTTTTTCACTGCATTCTAGTTGT
GGTTTGTCCAAACTCATCAATGTATCTTATCATGTCTGTATACCGTCGACCTCTAGCTAGAGCTTGGCGTAATCATGGTCATAGCTGTTTCC
TGTGTGAAATTGTTATCCGCTCACAATTCCACACAACATACGAGCCGGAAGCATAAAGTGTAAAGCCTGGGGTGCCTAATGAGTGAGCTAAC
TCACATTAATTGCGTTGCGCTCACTGCCCGCTTTCCAGTCGGGAAACCTGTCGTGCCAGCTGCATTAATGAATCGGCCAACGCGCGGGGAGA
GGCGGTTTGCGTATTGGGCGCTCTTCCGCTTCCTCGCTCACTGACTCGCTGCGCTCGGTCGTTCGGCTGCGGCGAGCGGTATCAGCTCACTC
AAAGGCGGTAATACGGTTATCCACAGAATCAGGGGATAACGCAGGAAAGAACATGTGAGCAAAAGGCCAGCAAAAGGCCAGGAACCGTAAAA
AGGCCGCGTTGCTGGCGTTTTTCCATAGGCTCCGCCCCCCTGACGAGCATCACAAAAATCGACGCTCAAGTCAGAGGTGGCGAAACCCGACA
GGACTATAAAGATACCAGGCGTTTCCCCCTGGAAGCTCCCTCGTGCGCTCTCCTGTTCCGACCCTGCCGCTTACCGGATACCTGTCCGCCTT
TCTCCCTTCGGGAAGCGTGGCGCTTTCTCAATGCTCACGCTGTAGGTATCTCAGTTCGGTGTAGGTCGTTCGCTCCAAGCTGGGCTGTGTGC
ACGAACCCCCCGTTCAGCCCGACCGCTGCGCCTTATCCGGTAACTATCGTCTTGAGTCCAACCCGGTAAGACACGACTTATCGCCACTGGCA
GCAGCCACTGGTAACAGGATTAGCAGAGCGAGGTATGTAGGCGGTGCTACAGAGTTCTTGAAGTGGTGGCCTAACTACGGCTACACTAGAAG
GACAGTATTTGGTATCTGCGCTCTGCTGAAGCCAGTTACCTTCGGAAAAAGAGTTGGTAGCTCTTGATCCGGCAAACAAACCACCGCTGGTA
GCGGTGGTTTTTTTGTTTGCAAGCAGCAGATTACGCGCAGAAAAAAAGGATCTCAAGAAGATCCTTTGATCTTTTCTACGGGGTCTGACGCT
CAGTGGAACGAAAACTCACGTTAAGGGATTTTGGTCATGAGATTATCAAAAAGGATCTTCACCTAGATCCTTTTAAATTAAAAATGAAGTTT
TAAATCAATCTAAAGTATATATGAGTAAACTTGGTCTGACAGTTACCAATGCTTAATCAGTGAGGCACCTATCTCAGCGATCTGTCTATTTC
GTTCATCCATAGTTGCCTGACTCCCCGTCGTGTAGATAACTACGATACGGGAGGGCTTACCATCTGGCCCCAGTGCTGCAATGATACCGCGA
GACCCACGCTCACCGGCTCCAGATTTATCAGCAATAAACCAGCCAGCCGGAAGGGCCGAGCGCAGAAGTGGTCCTGCAACTTTATCCGCCTC
CATCCAGTCTATTAATTGTTGCCGGGAAGCTAGAGTAAGTAGTTCGCCAGTTAATAGTTTGCGCAACGTTGTTGCCATTGCTACAGGCATCG
TGGTGTCACGCTCGTCGTTTGGTATGGCTTCATTCAGCTCCGGTTCCCAACGATCAAGGCGAGTTACATGATCCCCCATGTTGTGCAAAAAA
GCGGTTAGCTCCTTCGGTCCTCCGATCGTTGTCAGAAGTAAGTTGGCCGCAGTGTTATCACTCATGGTTATGGCAGCACTGCATAATTCTCT
TACTGTCATGCCATCCGTAAGATGCTTTTCTGTGACTGGTGAGTACTCAACCAAGTCATTCTGAGAATAGTGTATGCGGCGACCGAGTTGCT
CTTGCCCGGCGTCAATACGGGATAATACCGCGCCACATAGCAGAACTTTAAAAGTGCTCATCATTGGAAAACGTTCTTCGGGGCGAAAACTC
TCAAGGATCTTACCGCTGTTGAGATCCAGTTCGATGTAACCCACTCGTGCACCCAACTGATCTTCAGCATCTTTTACTTTCACCAGCGTTTC
TGGGTGAGCAAAAACAGGAAGGCAAAATGCCGCAAAAAAGGGAATAAGGGCGACACGGAAATGTTGAATACTCATACTCTTCCTTTTTCAAT
ATTATTGAAGCATTTATCAGGGTTATTGTCTCATGAGCGGATACATATTTGAATGTATTTAGAAAAATAAACAAATAGGGGTTCCGCGCACA
TTTCCCCGAAAAGTGCCACCTGACGTC
SEQ ID NO: 13
atggctcg tgcggtcggg atcgacctcg ggaccaccaa ctccgtcgtc tcggttctgg aaggtggcga cccggtcgtc
gtcgccaact ccgagggctc caggaccacc ccgtcaattg tcgcgttcgc ccgcaacggt gaggtgctgg tcggccagcc
cgccaagaac caggcagtga ccaacgtcga tcgcaccgtg cgctcggtca agcgacacat gggcagcgac tggtccatag
agattgacgg caagaaatac accgcgccgg agatcagcgc ccgcattctg atgaagctga agcgcgacgc cgaggcctac
ctcggtgagg acattaccga cgcggttatc acgacgcccg cctacttcaa tgacgcccag cgtcaggcca ccaaggacgc
cggccagatc gccggcctca acgtgctgcg gatcgtcaac gagccgaccg cggccgcgct ggcctacggc ctcgacaagg
gcgagaagga gcagcgaatc ctggtcttcg acttgggtgg tggcactttc gacgtttccc tgctggagat cggcgagggt
gtggttgagg tccgtgccac ttcgggtgac aaccacctcg gcggcgacga ctgggaccag cgggtcgtcg attggctggt
ggacaagttc aagggcacca gcggcatcga tctgaccaag gacaagatgg cgatgcagcg gctgcgggaa gccgccgaga
aggcaaagat cgagctgagt tcgagtcagt ccacctcgat caacctgccc tacatcaccg tcgacgccga caagaacccg
ttgttcttag acgagcagct gacccgcgcg gagttccaac ggatcactca ggacctgctg gaccgcactc gcaagccgtt
ccagtcggtg atcgctgaca ccggcatttc ggtgtcggag atcgatcacg ttgtgctcgt gggtggttcg acccggatgc
ccgcggtgac cgatctggtc aaggaactca ccggcggcaa ggaacccaac aagggcgtca accccgatga ggttgtcgcg
gtgggagccg ctctgcaggc cggcgtcctc aagggcgagg tgaaagacgt tctgctgctt gatgttaccc cgctgagcct
gggtatcgag accaagggcg gggtgatgac caggctcatc gagcgcaaca ccacgatccc caccaagcgg tcggagactt
tcaccaccgc cgacgacaac caaccgtcgg tgcagatcca ggtctatcag ggggagcgtg agatcgccgc gcacaacaag
ttgctcgggt ccttcgagct gaccggcatc ccgccggcgc cgcgggggat tccgcagatc gaggtcactt tcgacatcga
cgccaacggc attgtgcacg tcaccgccaa ggacaagggc accggcaagg agaacacgat ccgaatccag gaaggctcgg
gcctgtccaa ggaagacatt gaccgcatga tcaaggacgc cgaagcgcac gccgaggagg atcgcaagcg tcgcgaggag
gccgatgttc gtaatcaagc cgagacattg gtctaccaga cggagaagtt cgtcaaagaa cagcgtgagg ccgagggtgg
ttcgaaggta cctgaagaca cgctgaacaa ggttgatgcc gcggtggcgg aagcgaaggc ggcacttggc ggatcggata
tttcggccat caagtcggcg atggagaagc tgggccagga gtcgcaggct ctggggcaag cgatctacga agcagctcag
gctgcgtcac aggccactgg cgctgcccac cccggcggcg agccgggcgg tgcccacccc ggctcggctg atgacgttgt
ggacgcggag gtggtcgacg acggccggga ggccaagtga
SEQ ID NO: 14
MARAVGIDLG TTNSVVSVLE GGDPVVVANS EGSRTTPSIV AFARNGEVLV GQPAKNQAVT NVDRTVRSVK RHMGSDWSIE
IDGKKYTAPE ISARILMKLK RDAEAYLGED ITDAVITTPA YFNDAQRQAT KDAGQIAGLN VLRIVNEPTA AALAYGLDKG
EKEQRILVFD LGGGTFDVSL LEIGEGVVEV RATSGDNHLG GDDWDQRVVD WLVDKFKGTS GIDLTKDKMA MQRLREAAEK
AKIELSSSQS TSINLPYITV DADKNPLFLD EQLTRAEFQR ITQDLLDRTR KPFQSVIADT GISVSEIDHV VLVGGSTRMP
AVTDLVKELT GGKEPNKGVN PDEVVAVGAA LQAGVLKGEV KDVLLLDVTP LSLGIETKGG VMTRLIERNT TIPTKRSETF
TTADDNQPSV QIQVYQGERE IAAHNKLLGS FELTGIPPAP RGIPQIEVTF DIDANGIVHV TAKDKGTGKE NTIRIQEGSG
LSKEDIDRMI KDAEAHAEED RKRREEADVR NQAETLVYQT EKFVKEQREA EGGSKVPEDT LNKVDAAVAE AKAALGGSDI
SAIKSAMEKL GQESQALGQA IYEAAQAASQ ATGAAHPGGE PGGAHPGSAD DVVDAEVVDD GREAK
SEQ ID NO: 15
1/1 31/11
ATG CAT GGA GAT ACA CCT ACA TTG CAT GAA TAT ATG TTA GAT TTG CAA CCA GAG ACA ACT
61/21 91/31
GAT CTC TAC TGT TAT GAG CAA TTA AAT GAC AGC TCA GAG GAG GAG GAT GAA ATA GAT GGT
121/41 151/51
CCA GCT GGA CAA GCA GAA CCG GAC AGA GCC CAT TAC AAT ATT GTA ACC TTT TGT TGC AAG
181/61 211/71
TGT GAC TCT ACG CTT CGG TTG TGC GTA CAA AGC ACA CAC GTA GAC ATT CGT ACT TTG GAA
241/81 271/91
GAC CTG TTA ATG GGC ACA CTA GGA ATT GTG TGC CCC ATC TGT TCT CAA GGA TCC atg gct
301/101 331/111
cgt gcg gtc ggg atc gac ctc ggg acc acc aac tcc gtc gtc tcg gtt ctg gaa ggt ggc
361/121 391/131
gac ccg gtc gtc gtc gcc aac tcc gag ggc tcc agg acc acc ccg tca att gtc gcg ttc
421/141 451/151
gcc cgc aac ggt gag gtg ctg gtc ggc cag ccc gcc aag aac cag gca gtg acc aac gtc
481/161 511/171
gat cgc acc gtg cgc tcg gtc aag cga cac atg ggc agc gac tgg tcc ata gag att gac
541/181 571/191
ggc aag aaa tac acc gcg ccg gag atc agc gcc cgc att ctg atg aag ctg aag cgc gac
601/201 631/211
gcc gag gcc tac ctc ggt gag gac att acc gac gcg gtt atc acg acg ccc gcc tac ttc
661/221 691/231
aat gac gcc cag cgt cag gcc acc aag gac gcc ggc cag atc gcc ggc ctc aac gtg ctg
721/241 751/251
cgg atc gtc aac gag ccg acc gcg gcc gcg ctg gcc tac ggc ctc gac aag ggc gag aag
781/261 811/271
gag cag cga atc ctg gtc ttc gac ttg ggt ggt ggc act ttc gac gtt tcc ctg ctg gag
841/281 871/291
atc ggc gag ggt gtg gtt gag gtc cgt gcc act tcg ggt gac aac cac ctc ggc ggc gac
901/301 931/311
gac tgg gac cag cgg gtc gtc gat tgg ctg gtg gac aag ttc aag ggc acc agc ggc atc
961/321 991/331
gat ctg acc aag gac aag atg gcg atg cag cgg ctg cgg gaa gcc gcc gag aag gca aag
1021/341 1051/351
atc gag ctg agt tcg agt cag tcc acc tcg atc aac ctg ccc tac atc acc gtc gac gcc
1081/361 1111/371
gac aag aac ccg ttg ttc tta gac gag cag ctg acc cgc gcg gag ttc caa cgg atc act
1141/381 1171/391
cag gac ctg ctg gac cgc act cgc aag ccg ttc cag tcg gtg atc gct gac acc ggc att
1201/401 1231/411
tcg gtg tcg gag atc gat cac gtt gtg ctc gtg ggt ggt tcg acc cgg atg ccc gcg gtg
1261/421 1291/431
acc gat ctg gtc aag gaa ctc acc ggc ggc aag gaa ccc aac aag ggc gtc aac ccc gat
1321/441 1351/451
gag gtt gtc gcg gtg gga gcc gct ctg cag gcc ggc gtc ctc aag ggc gag gtg aaa gac
1381/461 1411/471
gtt ctg ctg ctt gat gtt acc ccg ctg agc ctg ggt atc gag acc aag ggc ggg gtg atg
1441/481 1471/491
acc agg ctc atc gag cgc aac acc acg atc ccc acc aag cgg tcg gag act ttc acc acc
1501/501 1531/511
gcc gac gac aac caa ccg tcg gtg cag atc cag gtc tat cag ggg gag cgt gag atc gcc
1561/521 1591/531
gcg cac aac aag ttg ctc ggg tcc ttc gag ctg acc ggc atc ccg ccg gcg ccg cgg ggg
1621/541 1651/551
att ccg cag atc gag gtc act ttc gac atc gac gcc aac ggc att gtg cac gtc acc gcc
1681/561 1711/571
aag gac aag ggc acc ggc aag gag aac acg atc cga atc cag gaa ggc tcg ggc ctg tcc
1741/581 1771/591
aag gaa gac att gac cgc atg atc aag gac gcc gaa gcg cac gcc gag gag gat cgc aag
1801/601 1831/611
cgt cgc gag gag gcc gat gtt cgt aat caa gcc gag aca ttg gtc tac cag acg gag aag
1861/621 1891/631
ttc gtc aaa gaa cag cgt gag gcc gag ggt ggt tcg aag gta cct gaa gac acg ctg aac
1921/641 1951/651
aag gtt gat gcc gcg gtg gcg gaa gcg aag gcg gca ctt ggc gga tcg gat att tcg gcc
1981/661 2011/671
atc aag tcg gcg atg gag aag ctg ggc cag gag tcg cag gct ctg ggg caa gcg atc tac
2041/681 2071/691
gaa gca gct cag gct gcg tca cag gcc act ggc gct gcc cac ccc ggc tcg gct gat gaA
2101/701
AGC a
SEQ ID NO: 16
1/1 31/11
Met His Gly Asp Thr Pro Thr Leu His Glu Tyr Met Leu Asp Leu Gln Pro Glu Thr Thr
61/21 91/31
Asp Leu Tyr Cys Tyr Glu Gln Leu Asn Asp Ser Ser Glu Glu Glu Asp Glu Ile Asp Gly
121/41 151/51
Pro Ala Gly Gln Ala Glu Pro Asp Arg Ala His Tyr Asn Ile Val Thr Phe Cys Cys Lys
181/61 211/71
Cys Asp Ser Thr Leu Arg Leu Cys Val Gln Ser Thr His Val Asp Ile Arg Thr Leu Glu
241/81 271/91
Asp Leu Leu Met Gly Thr Leu Gly Ile Val Cys Pro Ile Cys Ser Gln Gly Ser Met ala
301/101 331/111
Arg Ala Val Gly Ile Asp Leu Gly Thr Thr Asn Ser Val Val Ser Val Leu Glu Gly Gly
361/121 391/131
Asp Pro Val Val Val Ala Asn Ser Glu Gly Ser Arg Thr Thr Pro Ser Ile Val Ala Phe
421/141 451/151
Ala Arg Asn Gly Glu Val Leu Val Gly Gln Pro Ala Lys Asn Gln Ala Val Thr Asn Val
481/161 511/171
Asp Arg Thr Val Arg Ser Val Lys Arg His Met Gly Ser Asp Trp Ser Ile Glu Ile Asp
541/181 571/191
Gly Lys Lys Tyr Thr Ala Pro Glu Ile Ser Ala Arg Ile Leu Met Lys Leu Lys Arg Asp
601/201 631/211
Ala Glu Ala Tyr Leu Gly Glu Asp Ile Thr Asp Ala Val Ile Thr Thr Pro Ala Tyr Phe
661/221 691/231
Asn Asp Ala Gln Arg Gln Ala Thr Lys Asp Ala Gly Gln Ile Ala Gly Leu Asn Val Leu
721/241 751/251
Arg Ile Val Asn Glu Pro Thr Ala Ala Ala Leu Ala Tyr Gly Leu Asp Lys Gly Glu Lys
781/261 811/271
Glu Gln Arg Ile Leu Val Phe Asp Leu Gly Gly Gly Thr Phe Asp Val Ser Leu Leu Glu
841/281 871/291
Ile Gly Glu Gly Val Val Glu Val Arg Ala Thr Ser Gly Asp Asn His Leu Gly Gly Asp
901/301 931/311
Asp Trp Asp Gln Arg Val Val Asp Trp Leu Val Asp Lys Phe Lys Gly Thr Ser Gly Ile
961/321 991/331
Asp Leu Thr Lys Asp Lys Met ala Met Gln Arg Leu Arg Glu Ala Ala Glu Lys Ala Lys
1021/341 1051/351
Ile Glu Leu Ser Ser Ser Gln Ser Thr Ser Ile Asn Leu Pro Tyr Ile Thr Val Asp Ala
1081/361 1111/371
Asp Lys Asn Pro Leu Phe Leu Asp Glu Gln Leu Thr Arg Ala Glu Phe Gln Arg Ile Thr
1141/381 1171/391
Gln Asp Leu Leu Asp Arg Thr Arg Lys Pro Phe Gln Ser Val Ile Ala Asp Thr Gly Ile
1201/401 1231/411
Ser Val Ser Glu Ile Asp His Val Val Leu Val Gly Gly Ser Thr Arg Met Pro Ala Val
1261/421 1291/431
Thr Asp Leu Val Lys Glu Leu Thr Gly Gly Lys Glu Pro Asn Lys Gly Val Asn Pro Asp
1321/441 1351/451
Glu Val Val Ala Val Gly Ala Ala Leu Gln Ala Gly Val Leu Lys Gly Glu Val Lys Asp
1381/461 1411/471
Val Leu Leu Leu Asp Val Thr Pro Leu Ser Leu Gly Ile Glu Thr Lys Gly Gly Val Met
1441/481 1471/491
Thr Arg Leu Ile Glu Arg Asn Thr Thr Ile Pro Thr Lys Arg Ser Glu Thr Phe Thr Thr
1501/501 1531/511
Ala Asp Asp Asn Gln Pro Ser Val Gln Ile Gln Val Tyr Gln Gly Glu Arg Glu Ile Ala
1561/521 1591/531
Ala His Asn Lys Leu Leu Gly Ser Phe Glu Leu Thr Gly Ile Pro Pro Ala Pro Arg Gly
1621/541 1651/551
Ile Pro Gln Ile Glu Val Thr Phe Asp Ile Asp Ala Asn Gly Ile Val His Val Thr Ala
1681/561 1711/571
Lys Asp Lys Gly Thr Gly Lys Glu Asn Thr Ile Arg Ile Gln Glu Gly Ser Gly Leu Ser
1741/581 1771/591
Lys Glu Asp Ile Asp Arg Met Ile Lys Asp Ala Glu Ala His Ala Glu Glu Asp Arg Lys
1801/601 1831/611
Arg Arg Glu Glu Ala Asp Val Arg Asn Gln Ala Glu Thr Leu Val Tyr Gln Thr Glu Lys
1861/621 1891/631
Phe Val Lys Glu Gln Arg Glu Ala Glu Gly Gly Ser Lys Val Pro Glu Asp Thr Leu Asn
1921/641 1951/651
Lys Val Asp Ala Ala Val Ala Glu Ala Lys Ala Ala Leu Gly Gly Ser Asp Ile Ser Ala
1981/661 2011/671
Ile Lys Ser Ala Met Glu Lys Leu Gly Gln Glu Ser Gln Ala Leu Gly Gln Ala Ile Tyr
2041/681 2071/691
GLU ALA ALA GLN ALA ALA SER GLN ALA THR GLY ALA ALA HIS PRO GLY SER ALA ASP GLU
2101/701
Ser

| | |
|---|---|
| SEQ ID NO: 17 | |
| ctgcagctgg tcaggccgtt tccgcaacgc ttgaagtcct ggccgatata ccggcagggc cagccatcgt tcgacgaata | |
| aagccacctc agccatgatg ccctttccat ccccagcgga accccgacat ggacgccaaa gccctgctcc tcggcagcct | |
| ctgcctggcc gccccattcg ccgacgcggc gacgctcgac aatgctctct ccgcctgcct cgccgcccgg ctcggtgcac | |
| cgcacacggc ggagggccag ttgcacctgc cactcaccct tgaggcccgg cgctccaccg gcgaatgcgg ctgtacctcg | |
| gcgctggtgc gatatcggct gctggccagg ggcgccagcg ccgacagcct cgtgcttcaa gagggctgct cgatagtcgc | |
| caggacacgc cgcgcacgct gaccctggcg gcggacgccg gcttggcgag cggccgcgaa ctggtcgtca ccctgggttg | |
| tcaggcgcct gactgacagg ccgggctgcc accaccaggc cgagatggac gccctgcatg tatcctccga tcggcaagcc | |
| tcccgttcgc acattcacca ctctgcaatc cagttcataa atcccataaa agccctcttc cgctccccgc cagcctcccc | |
| gcatcccgca ccctagacgc cccgccgctc tccgccggct cgcccgacaa gaaaaaccaa ccgctcgatc agcctcatcc | |
| ttcacccatc acaggagcca tcgcgatgca cctgataccc cattggatcc ccctggtcgc cagcctcggc ctgctcgccg | |
| gcggctcgtc cgcgtccgcc gccgaggaag ccttcgacct ctggaacgaa tgcgccaaag cctgcgtgct cgacctcaag | |
| gacggcgtgc gttccagccg catgagcgtc gacccggcca tcgccgacac caacggccag ggcgtgctgc actactccat | |
| ggtcctggag ggcggcaacg acgcgctcaa gctggccatc gacaacgccc tcagcatcac cagcgacggc ctgaccatcc | |
| gcctcgaagg cggcgtcgag ccgaacaagc cggtgcgcta cagctacacg cgccaggcgc gcggcagttg gtcgctgaac | |
| tggctggtac cgatcggcca cgagaagccc tcgaacatca aggtgttcat ccacgaactg aacgccggca accagctcag | |
| ccacatgtcg ccgatctaca ccatcgagat gggcgacgag ttgctggcga agctggcgcg cgatgccacc ttcttcgtca | |
| gggcgcacga gagcaacgag atgcagccga cgctcgccat cagccatgcc ggggtcagcg tggtcatggc ccagacccag | |
| ccgcgccggg aaaagcgctg gagcgaatgg gccagcggca aggtgttgtg cctgctcgac ccgctggacg gggtctacaa | |
| ctacctcgcc cagcaacgct gcaacctcga cgatacctgg gaaggcaaga tctaccgggt gctcgccggc aacccggcga | |
| agcatgacct ggacatcaaa cccacggtca tcagtcatcg cctgcacttt cccgagggcg gcagcctggc cgcgctgacc | |
| gcgcaccagg cttgccacct gccgctggag actttcaccc gtcatcgcca gccgcgcggc tgggaacaac tggagcagtg | |
| cggctatccg gtgcagcggc tggtcgccct ctacctggcg gcgcggctgt cgtggaacca ggtcgaccag gtgatccgca | |
| acgccctggc cagccccggc agcggcggcg acctgggcga agcgatccgc gagcagccgg agcaggcccg tctggccctg | |
| accctggccg ccgccgagag cgagcgcttc gtccggcagg gcaccggcaa cgacgaggcc ggcgcggcca acgccgacgt | |
| ggtgagcctg acctgcccgg tcgccgccgg tgaatgcgcg ggcccggcgg acagcggcga cgccctgctg gagcgcaact | |
| atcccactgg cgcggagttc ctcggcgacg gcggcgacgt cagcttcagc acccgcggca cgcagaactg gacggtggag | |
| cggctgctcc aggcgcaccg ccaactggag gagcgcggct atgtgttcgt cggctaccac ggcaccttcc tcgaagcggc | |
| gcaaagcatc gtcttcggcg gggtgcgcgc gcgcagccag gacctcgacg cgatctggcg cggtttctat atcgccggcg | |
| atccggcgct ggcctacggc tacgcccagg accaggaacc cgacgcacgc ggccggatcc gcaacggtgc cctgctgcgg | |
| gtctatgtgc cgcgctcgag cctgccgggc ttctaccgca ccagcctgac cctggccgcg ccggaggcgg cgggcgaggt | |
| cgaacggctg atcggccatc cgctgccgct gcgcctggac gccatcaccg gccccgagga ggaaggcggg cgcctggaga | |
| ccattctcgg ctggccgctg gccgagcgca ccgtggtgat tccctcggcg atccccaccg acccgcgcaa cgtcggcggc | |
| gacctcgacc cgtccagcat ccccgacaag gaacaggcga tcagcgccct gccggactac gccagccagc ccggcaaacc | |
| gccgcgcgag gacctgaagt aactgccgcg accggccggc tcccttcgca ggagccggcc ttctcggggc ctggccatac | |
| atcaggtttt cctgatgcca gcccaatcga atatgaattc | 2760 |
| SEQ ID NO: 18 | |
| MHLIPHWIPL VASLGLLAGG SSASAAEEAF DLWNECAKAC VLDLKDGVRS SRMSVDPAIA DTNGQGVLHY SMVLEGGNDA | |
| LKLAIDNALS ITSDGLTIRL EGGVEPNKPV RYSYTRQARG SWSLNWLVPI GHEKPSNIKV FIHELNAGNQ LSHMSPIYTI | |
| EMGDELLAKL ARDATFFVRA HESNEMQPTL AISHAGVSVV MAQTQPRREK RWSEWASGKV LCLLDPLDGV YNYLAQQRCN | |
| LDDTWEGKIY RVLAGNPAKH DLDIKPTVIS HRLHFPEGGS LAALTAHQAC HLPLETFTRH RQPRGWEQLE QCGYPVQRLV | |
| ALYLAARLSW NQVDQVIRNA LASPGSGGDL GEAIREQPEQ ARLALTLAAA ESERFVRQGT GNDEAGAANA DVVSLTCPVA | |
| AGECAGPADS GDALLERNYP TGAEFLGDGG DVSFSTRGTQ NWTVERLLQA HRQLEERGYV FVGYHGTFLE AAQSIVFGGV | |
| RARSQDLDAI WRGFYIAGDP ALAYGYAQDQ EPDARGRIRN GALLRVYVPR SSLPGFYRTS LTLAAPEAAG EVERLIGHPL | |
| PLRLDAITGP EEEGGRLETI LGWPLAERTV VIPSAIPTDP RNVGGDLDPS SIPDKEQAIS ALPDYASQPG KPPREDLK | 638 |
| SEQ ID NO: 19 | |
| RLHFPEGGSL AALTAHQACH LPLETFTRHR QPRGWEQLEQ CGYPVQRLVA LYLAARLSWN QVDQVIRNAL ASPGSGGDLG | |
| EAIREQPEQA RLALTLAAAE SERFVRQGTG NDEAGAANAD VVSLTCPVAA GECAGPADSG DALLERNYPT GAEFLGDGGD | |
| VSFSTRGTQN W | 171 |

SEQ ID NO: 20
1/1 31/11
atg cgc ctg cac ttt ccc gag ggc ggc agc ctg gcc gcg ctg acc gcg cac cag gct tgc
61/21 91/31
cac ctg ccg ctg gag act ttc acc cgt cat cgc cag ccg cgc ggc tgg gaa caa ctg gag
121/41 151/51
cag tgc ggc tat ccg gtg cag cgg ctg gtc gcc ctc tac ctg gcg gcg cgg ctg tcg tgg
181/61 211/71
aac cag gtc gac cag gtg atc cgc aac gcc ctg gcc agc ccc ggc agc ggc ggc gac ctg
241/81 271/91
ggc gaa gcg atc cgc gag cag ccg gag cag gcc cgt ctg gcc ctg acc ctg gcc gcc gcc
301/101 331/111
gag agc gag cgc ttc gtc cgg cag ggc acc ggc aac gac gag gcc ggc gcg gcc aac gcc
361/121 391/131
gac gtg gtg agc ctg acc tgc ccg gtc gcc gcc ggt gaa tgc gcg ggc ccg gcg gac agc
421/141 451/151
ggc gac gcc ctg ctg gag cgc aac tat ccc act ggc gcg gag ttc ctc ggc gac ggc ggc
481/161 511/171
gac gtc agc ttc agc acc cgc ggc acg cag [symbol] atg cat gga gat aca cct aca
541/181 571/191
ttg cat gaa tat atg tta gat ttg caa cca gag aca act gat ctc tac tgt tat gag caa
601/201 631/211
tta aat gac agc tca gag gag gag gat gaa ata gat ggt cca gct gga caa gca gaa ccg
661/221 691/231
gac aga gcc cat tac aat att gta acc ttt tgt tgc aag tgt gac tct acg ctt cgg ttg
721/241 751/251
tgc gta caa agc aca cac gta gac att cgt act ttg gaa gac ctg tta atg ggc aca cta
781/261 811/271
gga att gtg tgc ccc atc tgt tct caa gga tcc gag ctc ggt acc aag ctt aag ttt aaa
841/281
ccg ctg atc agc ctc gac tgt gcc ttc tag
SEQ ID NO: 21
1/1 31/11
Met arg leu his phe pro glu gly gly ser leu ala ala leu thr ala his gln ala cys
61/21 91/31
His Leu Pro Leu Glu Thr Phe Thr Arg His Arg Gln Pro Arg Gly Trp Glu Gln Leu Glu
121/41 151/51
Gln Cys Gly Tyr Pro Val Gln Arg Leu Val Ala Leu Tyr Leu Ala Ala Arg Leu Ser Trp
181/61 211/71
Asn Gln Val Asp Gln Val Ile Arg Asn Ala Leu Ala Ser Pro Gly Ser Gly Gly Asp Leu
241/81 271/91
Gly Glu Ala Ile Arg Glu Gln Pro Glu Gln Ala Arg Leu Ala Leu Thr Leu Ala Ala Ala
301/101 331/111
Glu Ser Glu Arg Phe Val Arg Gln Gly Thr Gly Asn Asp Glu Ala Gly Ala Ala Asn Ala
361/121 391/131
Asp Val Val Ser Leu Thr Cys Pro Val Ala Ala Gly Glu Cys Ala Gly Pro Ala Asp Ser
421/141 451/151
Gly Asp Ala Leu Leu Glu Arg Asn Tyr Pro Thr Gly Ala Glu Phe Leu Gly Asp Gly Gly
481/161 511/171
Asp Val Ser Phe Ser Thr Arg Gly Thr Gln [symbol] Met His Gly Asp Thr Pro Thr
541/181 571/191
Leu His Glu Tyr Met Leu Asp Leu Gln Pro Glu Thr Thr Asp Leu Tyr Cys Tyr Glu Gln
601/201 631/211
Leu Asn Asp Ser Ser Glu Glu Glu Asp Glu Ile Asp Gly Pro Ala Gly Gln Ala Glu Pro
661/221 691/231
Asp Arg Ala His Tyr Asn Ile Val Thr Phe Cys Cys Lys Cys Asp Ser Thr Leu Arg Leu
721/241 751/251
Cys Val Gln Ser Thr His Val Asp Ile Arg Thr Leu Glu Asp Leu Leu Met Gly Thr Leu
781/261 811/271
Gly Ile Val Cys Pro Ile Cys Ser Gln Gly Ser Glu Leu Gly Thr Lys Leu Lys Phe Lys
841/281

| | |
|---|---|
| SEQ ID NO: 22 (coded protein disclosed as SEQ ID NO: 37) | |
| atg acc tct cgc cgc tcc gtg aag tcg ggt ccg cgg gag gtt ccg cgc | 48 |
| Met Thr Ser Arg Arg Ser Val Lys Ser Gly Pro Arg Glu Val Pro Arg | |
| 1 5 10 15 | |
| gat gag tac gag gat ctg tac tac acc ccg tct tca ggt atg gcg agt | 96 |
| Asp Glu Tyr Glu Asp Leu Tyr Tyr Thr Pro Ser Ser Gly Met Ala Ser | |
| 20 25 30 | |
| ccc gat agt ccg cct gac acc tcc cgc cgt ggc gcc cta cag aca cgc | 144 |
| Pro Asp Ser Pro Pro Asp Thr Ser Arg Arg Gly Ala Leu Gln Thr Arg | |
| 35 40 45 | |
| tcg cgc cag agg ggc gag gtc cgt ttc gtc cag tac gac gag tcg gat | 192 |
| Ser Arg Gln Arg Gly Glu Val Arg Phe Val Gln Tyr Asp Glu Ser Asp | |
| 50 55 60 | |
| tat gcc ctc tac ggg ggc tcg tct tcc gaa gac gac gaa cac ccg gag | 240 |
| Tyr Ala Leu Tyr Gly Gly Ser Ser Ser Glu Asp Asp Glu His Pro Glu | |
| 65 70 75 80 | |
| gtc ccc cgg acg cgg cgt ccc gtt tcc ggg gcg gtt ttg tcc ggc ccg | 288 |
| Val Pro Arg Thr Arg Arg Pro Val Ser Gly Ala Val Leu Ser Gly Pro | |
| 85 90 95 | |
| ggg cct gcg cgg gcg cct ccg cca ccc gct ggg tcc gga ggg gcc gga | 336 |
| Gly Pro Ala Arg Ala Pro Pro Pro Pro Ala Gly Ser Gly Gly Ala Gly | |
| 100 105 110 | |
| cgc aca ccc acc acc gcc ccc cgg gcc ccc cga acc cag cgg gtg gcg | 384 |
| Arg Thr Pro Thr Thr Ala Pro Arg Ala Pro Arg Thr Gln Arg Val Ala | |
| 115 120 125 | |
| tct aag gcc ccc gcg gcc ccg gcg gcg gag acc acc cgc ggc agg aaa | 432 |
| Ser Lys Ala Pro Ala Ala Pro Ala Ala Glu Thr Thr Arg Gly Arg Lys | |
| 130 135 140 | |
| tcg gcc cag cca gaa tcc gcc gca ctc cca gac gcc ccc gcg tcg acg | 480 |
| Ser Ala Gln Pro Glu Ser Ala Ala Leu Pro Asp Ala Pro Ala Ser Thr | |
| 145 150 155 160 | |
| gcg cca acc cga tcc aag aca ccc gcg cag ggg ctg gcc aga aag ctg | 528 |
| Ala Pro Thr Arg Ser Lys Thr Pro Ala Gln Gly Leu Ala Arg Lys Leu | |
| 165 170 175 | |
| cac ttt agc acc gcc ccc cca aac ccc gac gcg cca tgg acc ccc cgg | 576 |
| His Phe Ser Thr Ala Pro Pro Asn Pro Asp Ala Pro Trp Thr Pro Arg | |
| 180 185 190 | |
| gtg gcc ggc ttt aac aag cgc gtc ttc tgc gcc gcg gtc ggg cgc ctg | 624 |
| Val Ala Gly Phe Asn Lys Arg Val Phe Cys Ala Ala Val Gly Arg Leu | |
| 195 200 205 | |
| gcg gcc atg cat gcc cgg atg gcg gct gtc cag ctc tgg gac atg tcg | 672 |
| Ala Ala Met His Ala Arg Met Ala Ala Val Gln Leu Trp Asp Met Ser | |
| 210 215 220 | |
| cgt ccg cgc aca gac gaa gac ctc aac gaa ctc ctt ggc atc acc acc | 720 |
| Arg Pro Arg Thr Asp Glu Asp Leu Asn Glu Leu Leu Gly Ile Thr Thr | |
| 225 230 235 240 | |
| atc cgc gtg acg gtc tgc gag ggc aaa aac ctg ctt cag cgc gcc aac | 768 |
| Ile Arg Val Thr Val Cys Glu Gly Lys Asn Leu Leu Gln Arg Ala Asn | |
| 245 250 255 | |
| gag ttg gtg aat cca gac gtg gtg cag gac gtc gac gcg gcc acg gcg | 816 |
| Glu Leu Val Asn Pro Asp Val Val Gln Asp Val Asp Ala Ala Thr Ala | |
| 260 265 270 | |
| act cga ggg cgt tct gcg gcg tcg cgc ccc acc gag cga cct cga gcc | 864 |
| Thr Arg Gly Arg Ser Ala Ala Ser Arg Pro Thr Glu Arg Pro Arg Ala | |
| 275 280 285 | |
| cca gcc cgc tcc gct tct cgc ccc aga cgg ccc gtc gag ggt acc gag | 912 |
| Pro Ala Arg Ser Ala Ser Arg Pro Arg Arg Pro Val Glu Gly Thr Glu | |
| 290 295 300 | |
| ctc gga tcc atg cat gga gat aca cct aca ttg cat gaa tat atg tta | 960 |
| Leu Gly Ser Met His Gly Asp Thr Pro Thr Leu His Glu Tyr Met Leu | |
| 305 310 315 320 | |
| gat ttg caa cca gag aca act gat ctc tac tgt tat gag caa tta aat | 1008 |
| Asp Leu Gln Pro Glu Thr Thr Asp Leu Tyr Cys Tyr Glu Gln Leu Asn | |
| 325 330 335 | |
| gac agc tca gag gag gag gat gaa ata gat ggt cca gct gga caa gca | 1056 |
| Asp Ser Ser Glu Glu Glu Asp Glu Ile Asp Gly Pro Ala Gly Gln Ala | |
| 340 345 350 | |
| gaa ccg gac aga gcc cat tac aat att gta acc ttt tgt tgc aag tgt | 1104 |
| Glu Pro Asp Arg Ala His Tyr Asn Ile Val Thr Phe Cys Cys Lys Cys | |
| 355 360 365 | |
| gac tct acg ctt cgg ttg tgc gta caa agc aca cac gta gac att cgt | 1152 |
| Asp Ser Thr Leu Arg Leu Cys Val Gln Ser Thr His Val Asp Ile Arg | |
| 370 375 380 | |
| act ttg gaa gac ctg tta atg ggc aca cta gga att gtg tgc ccc atc | 1200 |
| Thr Leu Glu Asp Leu Leu Met Gly Thr Leu Gly Ile Val Cys Pro Ile | |
| 385 390 395 400 | |
| tgt tct cag gat aag ctt aag ttt aaa ccg ctg atc agc ctc gac tgt | 1248 |
| Cys Ser Gln Asp Lys Leu Lys Phe Lys Pro Leu Ile Ser Leu Asp Cys | |
| 405 410 415 | |
| gcc ttc tag | 1257 |
| Ala Phe | |
| SEQ ID NO: 23 | |

| | | |
|---|---|---|
| 1 | atgctgctat ccgtgccgct gctgctcggc ctcctcggcc tggccgtcgc cgagcccgcc | |
| 61 | gtctacttca aggagcagtt tctggacgga gacgggtgga cttcccgctg gatcgaatcc | |
| 121 | aaacacaagt cagattttgg caaattcgtt ctcagttccg gcaagttcta cggtgacgag | |
| 181 | gagaaagata aaggtttgca gacaagccag gatgcacgct tttatgctct gtcggccagt | |
| 241 | ttcgagcctt tcagcaacaa aggccagacg ctggtggtgc agttcacggt gaaacatgag | |
| 301 | cagaacatcg actgtggggg cggctatgtg aagctgtttc ctaatagttt ggaccagaca | |
| 361 | gacatgcacg gagactcaga atacaacatc atgtttggtc ccgacatctg tggccctggc | |
| 421 | accaagaagg ttcatgtcat cttcaactac aagggcaaga acgtgctgat caacaaggac | |
| 481 | atccgttgca aggatgatga gtttacacac ctgtacacac tgattgtgcg gccagacaac | |
| 541 | acctatgagg tgaagattga caacagccag gtggagtccg gctccttgga agacgattgg | |
| 601 | gacttcctgc cacccaagaa gataaaggat cctgatgctt caaaaccgga agactgggat | |
| 661 | gagcgggcca agatcgatga tcccacagac tccaagcctg aggactggga caagcccgag | |
| 721 | catatccctg accctgatgc taagaagccc gaggactggg atgaagagat ggacggagag | |
| 781 | tgggaacccc cagtgattca gaaccctgag tacaagggtg agtggaagcc ccggcagatc | |
| 841 | gacaacccag attacaaggg cacttggatc cacccagaaa ttgacaaccc cgagtattct | |
| 901 | cccgatccca gtatctatgc ctatgataac tttggcgtgc tgggcctgga cctctggcag | |
| 961 | gtcaagtctg gcaccatctt tgacaacttc ctcatcacca acgatgaggc atacgctgag | |
| 1021 | gagtttggca acgagacgtg gggcgtaaca aaggcagcag agaaacaaat gaaggacaaa | |
| 1081 | caggacgagg agcagaggct taaggaggag gaagaagaca agaaacgcaa agaggaggag | |
| 1141 | gaggcagagg acaaggagga tgatgaggac aaagatgagg atgaggagga tgaggaggac | |
| 1201 | aaggaggaag atgaggagga agatgtcccc ggccaggcca aggacgagct gtag | 1251 |

SEQ ID NO: 24

| | |
|---|---|
| 1 | MLLSVPLLLG LLGLAVAEPA VYFKEQFLDG DGWTSRWIES KHKSDFGKFV LSSGKFYGDE |
| 61 | EKDKGLQTSQ DARFYALSAS FEPFSNKGQT LVVQFTVKHE QNIDCGGGYV KLFPNSLDQT |
| 121 | DMHGDSEYNI MFGPDICGPG TKKVHVIFNY KGKNVLINKD IRCKDDEFTH LYTLIVRPDN |
| 181 | TYEVKIDNSQ VESGSLEDDW DFLPPKKIKD PDASKPEDWD ERAKIDDPTD SKPEDWDKPE |
| 241 | HIPDPDAKKP EDWDEEMDGE WEPPVIQNPE YKGEWKPRQ [symbol] |
| 301 | [symbol] [symbol] |
| 361 | [symbol] [symbol] |

SEQ ID NO: 25

| | | |
|---|---|---|
| 1 | MLLSVPLLLG LLGLAVAEPA VYFKEQFLDG DGWTSRWIES KHKSDFGKFV LSSGKFYGDE | |
| 61 | EKDKGLQTSQ DARFYALSAS FEPFSNKGQT LVVQFTVKHE QNIDCGGGYV KLFPNSLDQT | |
| 121 | DMHGDSEYNI MFGPDICGPG TKKVHVIFNY KGKNVLINKD IRCKDDEFTH | 170 |

SEQ ID NO: 26

| | | |
|---|---|---|
| 1 | LYTLIVRPDN TYEVKIDNSQ VESGSLEDDW DFLPPKKIKD PDASKPEDWD ERAKIDDPTD | |
| 61 | SKPEDWDKPE HIPDPDAKKP EDWDEEMDGE WEPPVIQNPE YKGEWKPRQ | 109 |

SEQ ID NO: 27

| | | |
|---|---|---|
| 1 | IDNPDYKGTW IHPEIDNPEY SPDPSIYAYD NFGVLGLDLW QVKSGTIFDN FLITNDEAYA | |
| 61 | EEFGNETWGV TKAAEKQMKD KQDEEQRLKE EEEDKKRKEE EEAEDKEDDE DKDEDEEDEE | |
| 121 | DKEEDEEEDV PGQAKDEL | 138 |

| | |
|---|---|
| SEQ ID NO: 28 | |
| 1 ATGCTGCTAT CCGTGCCGCT GCTGCTCGGC CTCCTCGGCC TGGCCGTCGC CGAGCCCGCC | |
| 61 GTCTACTTCA AGGAGCAGTT TCTGGACGGA GACGGGTGGA CTTCCCGCTG GATCGAATCC | |
| 121 AAACACAAGT CAGATTTTGG CAAATTCGTT CTCAGTTCCG GCAAGTTCTA CGGTGACGAG | |
| 181 GAGAAAGATA AAGGTTTGCA GACAAGCCAG GATGCACGCT TTTATGCTCT GTCGGCCAGT | |
| 241 TTCGAGCCTT TCAGCAACAA AGGCCAGACG CTGGTGGTGC AGTTCACGGT GAAACATGAG | |
| 301 CAGAACATCG ACTGTGGGGG CGGCTATGTG AAGCTGTTTC CTAATAGTTT GGACCAGACA | |
| 361 GACATGCACG GAGACTCAGA ATACAACATC ATGTTTGGTC CCGACATCTG TGGCCCTGGC | |
| 421 ACCAAGAAGG TTCATGTCAT CTTCAACTAC AAGGGCAAGA ACGTGCTGAT CAACAAGGAC | |
| 481 ATCCGTTGCA AGGATGATGA GTTTACACAC CTGTACACAC TGATTGTGCG GCCAGACAAC | |
| 541 acctatgagg tgaagattga caacagccag gtggagtccg gctccttgga agacgattgg | |
| 601 gacttcctgc cacccaagaa gataaaggat cctgatgctt caaaaccgga agactgggat | |
| 661 gagcgggcca agatcgatga tcccacagac tccaagcctg aggactggga caagcccgag | |
| 721 catatccctg accctgatgc taagaagccc gaggactggg atgaagagat ggacggagag | |
| 781 tgggaacccc cagtgattca gaaccctgag tacaagggtg agtggaagcc ccggcagatc | |
| 841 gacaacccag attacaaggg cacttggatc cacccagaaa ttgacaaccc cgagtattct | |
| 901 cccgatccca gtatctatgc ctatgataac tttggcgtgc tgggcctgga cctctggcag | |
| 961 gtcaagtctg gcaccatctt tgacaacttc ctcatcacca acgatgaggc atacgctgag | |
| 1021 gagtttggca acgagacgtg gggcgtaaca aaggcagcag agaaacaaat gaaggacaaa | |
| 1081 caggacgagg agcagaggct taaggaggag gaagaagaca agaaacgcaa agaggaggag | |
| 1141 gaggcagagg acaaggagga tgatgaggac aaagatgagg atgaggagga tgaggaggac | |
| 1201 aaggaggaag atgaggagga agatgtcccc ggccaggcca aggacgagct gtag | 1251 |
| SEQ ID NO: 29 | |

| | |
|---|---|
| 1 | ATGCTGCTAT CCGTGCCGCT GCTGCTCGGC CTCCTCGGCC TGGCCGTCGC CGAGCCCGCC |
| 61 | GTCTACTTCA AGGAGCAGTT TCTGGACGGA GACGGGTGGA CTTCCCGCTG GATCGAATCC |
| 121 | AAACACAAGT CAGATTTTGG CAAATTCGTT CTCAGTTCCG GCAAGTTCTA CGGTGACGAG |
| 181 | GAGAAAGATA AAGGTTTGCA GACAAGCCAG GATGCACGCT TTTATGCTCT GTCGGCCAGT |
| 241 | TTCGAGCCTT TCAGCAACAA AGGCCAGACG CTGGTGGTGC AGTTCACGGT GAAACATGAG |
| 301 | CAGAACATCG ACTGTGGGGG CGGCTATGTG AAGCTGTTTC CTAATAGTTT GGACCAGACA |
| 361 | GACATGCACG GAGACTCAGA ATACAACATC ATGTTTGGTC CCGACATCTG TGGCCCTGGC |
| 421 | ACCAAGAAGG TTCATGTCAT CTTCAACTAC AAGGGCAAGA ACGTGCTGAT CAACAAGGAC |
| 481 | ATCCGTTGCA AGGATGATGA GTTTACACAC CTGTACACAC TGATTGTGCG GCCAGACAAC |

SEQ ID NO: 30

| | | |
|---|---|---|
| 1 | acctatgagg tgaagattga caacagccag gtggagtccg gctccttgga agacgattgg | |
| 61 | gacttcctgc cacccaagaa gataaaggat cctgatgctt caaaaccgga agactgggat | |
| 121 | gagcgggcca agatcgatga tcccacagac tccaagcctg aggactggga caagcccgag | |
| 181 | catatccctg accctgatgc taagaagccc gaggactggg atgaagagat ggacggagag | |
| 241 | tgggaacccc cagtgattca gaaccct | 267 |

SEQ ID NO: 31

| | | |
|---|---|---|
| 1 | gagtacaagg gtgagtggaa gccccggcag atcgacaacc cagattacaa gggcacttgg | |
| 61 | atccacccag aaattgacaa ccccgagtat tctcccgatc ccagtatcta tgcctatgat | |
| 121 | aactttggcg tgctgggcct ggacctctgg caggtcaagt ctggcaccat ctttgacaac | |
| 181 | ttcctcatca ccaacgatga ggcatacgct gaggagtttg gcaacgagac gtggggcgta | |
| 241 | acaaaggcag cagagaaaca aatgaaggac aaacaggacg aggagcagag gcttaaggag | |
| 301 | gaggaagaag acaagaaacg caaagaggag gaggaggcag aggacaagga ggatgatgag | |
| 361 | gacaaagatg aggatgagga ggatgaggag gacaaggagg aagatgagga ggaagatgtc | |
| 421 | cccggccagg ccaaggacga gctg | 444 |

| | |
|---|---|
| SEQ ID NO: 32 | |
| 1 gctccgcccc cctgacgagc atcacaaaaa tcgacgctca agtcagaggt ggcgaaaccc | |
| 61 gacaggacta taaagatacc aggcgtttcc ccctggaagc tccctcgtgc gctctcctgt | |
| 121 tccgaccctg ccgcttaccg gatacctgtc cgcctttctc ccttcgggaa gcgtggcgct | |
| 181 ttctcatagc tcacgctgta ggtatctcag ttcggtgtag gtcgttcgct ccaagctggg | |
| 241 ctgtgtgcac gaaccccccg ttcagcccga ccgctgcgcc ttatccggta actatcgtct | |
| 301 tgagtccaac ccggtaagac acgacttatc gccactggca gcagccactg gtaacaggat | |
| 361 tagcagagcg aggtatgtag gcggtgctac agagttcttg aagtggtggc ctaactacgg | |
| 421 ctacactaga agaacagtat ttggtatctg cgctctgctg aagccagtta ccttcggaaa | |
| 481 aagagttggt agctcttgat ccggcaaaca aaccaccgct ggtagcggtg gtttttttgt | |
| 541 ttgcaagcag cagattacgc gcagaaaaaa aggatctcaa gaagatcctt tgatcttttc | |
| 601 tacggggtct gacgctcagt ggaacgaaaa ctcacgttaa gggattttgg tcatgagatt | |
| 661 atcaaaaagg atcttcacct agatcctttt aaattaaaaa tgaagtttta aatcaatcta | |
| 721 aagtatatat gagtaaactt ggtctgacag ttaccaatgc ttaatcagtg aggcacctat | |
| 781 ctcagcgatc tgtctatttc gttcatccat agttgcctga ctcggggggg gggggcgctg | |
| 841 aggtctgcct cgtgaagaag gtgttgctga ctcataccag ggcaacgttg ttgccattgc | |
| 901 tacaggcatc gtggtgtcac gctcgtcgtt tggtatggct tcattcagct ccggttccca | |
| 961 acgatcaagg cgagttacat gatcccccat gttgtgcaaa aaagcggtta gctccttcgg | |
| 1021 tcctccgatc gttgtcagaa gtaagttggc cgcagtgtta tcactcatgg ttatggcagc | |
| 1081 actgcataat tctcttactg tcatgccatc cgtaagatgc ttttctgtga ctggtgagta | |
| 1141 ctcaaccaag tcattctgag aatagtgtat gcggcgaccg agttgctctt gcccggcgtc | |
| 1201 aatacgggat aataccgcgc cacatagcag aactttaaaa gtgctcatca ttggaaaacg | |
| 1261 ttcttcgggg cgaaaactct caaggatctt accgctgttg agatccagtt cgatgtaacc | |
| 1321 cactcgtgca cctgaatcgc cccatcatcc agccagaaag tgagggagcc acggttgatg | |
| 1381 agagctttgt tgtaggtgga ccagttggtg attttgaact tttgctttgc cacggaacgg | |
| 1441 tctgcgttgt cgggaagatg cgtgatctga tccttcaact cagcaaaagt tcgatttatt | |
| 1501 caacaaagcc gccgtcccgt caagtcagcg taatgctctg ccagtgttac aaccaattaa | |
| 1561 ccaattctga ttagaaaaac tcatcgagca tcaaatgaaa ctgcaattta ttcatatcag | |
| 1621 gattatcaat accatatttt tgaaaaagcc gtttctgtaa tgaaggagaa aactcaccga | |
| 1681 ggcagttcca taggatggca agatcctggt atcggtctgc gattccgact cgtccaacat | |
| 1741 caatacaacc tattaatttc ccctcgtcaa aaataaggtt atcaagtgag aaatcaccat | |
| 1801 gagtgacgac tgaatccggt gagaatggca aaagcttatg catttctttc cagacttgtt | |
| 1861 caacaggcca gccattacgc tcgtcatcaa aatcactcgc atcaaccaaa ccgttattca | |
| 1921 ttcgtgattg cgcctgagcg agacgaaata cgcgatcgct gttaaaagga caattacaaa | |
| 1981 caggaatcga atgcaaccgg cgcaggaaca ctgccagcgc atcaacaata ttttcacctg | |
| 2041 aatcaggata ttcttctaat acctggaatg ctgttttccc ggggatcgca gtggtgagta | |
| 2101 accatgcatc atcaggagta cggataaaat gcttgatggt cggaagaggc ataaattccg | |
| 2161 tcagccagtt tagtctgacc atctcatctg taacatcatt ggcaacgcta cctttgccat | |
| 2221 gtttcagaaa caactctggc gcatcgggct tcccatacaa tcgatagatt gtcgcacctg | |
| 2281 attgcccgac attatcgcga gcccatttat acccatataa atcagcatcc atgttggaat | |
| 2341 ttaatcgcgg cctcgagcaa gacgtttccc gttgaatatg gctcataaca ccccttgtat | |
| 2401 tactgtttat gtaagcagac agttttattg ttcatgatga tatattttta tcttgtgcaa | |
| 2461 tgtaacatca gagattttga gacacaacgt ggctttcccc ccccccccat tattgaagca | |
| 2521 tttatcaggg ttattgtctc atgagcggat acatatttga atgtatttag aaaaataaac | |
| 2581 aaataggggt tccgcgcaca tttccccgaa aagtgccacc tgacgtctaa gaaaccatta | |
| 2641 ttatcatgac attaacctat aaaaataggc gtatcacgag gccctttcgt ctcgcgcgtt | |
| 2701 tcggtgatga cggtgaaaac ctctgacaca tgcagctccc ggagacggtc acagcttgtc | |
| 2761 tgtaagcgga tgccgggagc agacaagccc gtcagggcgc gtcagcgggt gttggcgggt | |
| 2821 gtcggggctg gcttaactat gcggcatcag agcagattgt actgagagtg caccatatgc | |
| 2881 ggtgtgaaat accgcacaga tgcgtaagga gaaaataccg catcagattg gctattggcc | |
| 2941 attgcatacg ttgtatccat atcataatat gtacatttat attggctcat gtccaacatt | |
| 3001 accgccatgt tgacattgat tattgactag ttattaatag taatcaatta cggggtcatt | |
| 3061 agttcatagc ccatatatgg agttccgcgt tacataactt acggtaaatg gcccgcctgg | |
| 3121 ctgaccgccc aacgaccccc gcccattgac gtcaataatg acgtatgttc ccatagtaac | |
| 3181 gccaataggg actttccatt gacgtcaatg ggtggagtat ttacggtaaa ctgcccactt | |
| 3241 ggcagtacat caagtgtatc atatgccaag tacgccccct attgacgtca atgacggtaa | |
| 3301 atggcccgcc tggcattatg cccagtacat gaccttatgg gactttccta cttggcagta | |
| 3361 catctacgta ttagtcatcg ctattaccat ggtgatgcgg ttttggcagt acatcaatgg | |
| 3421 gcgtggatag cggtttgact cacggggatt tccaagtctc caccccattg acgtcaatgg | |
| 3481 gagtttgttt tggcaccaaa atcaacggga ctttccaaaa tgtcgtaaca actccgcccc | |
| 3541 attgacgcaa atgggcggta ggcgtgtacg gtgggaggtc tatataagca gagctcgttt | |
| 3601 agtgaaccgt cagatcgcct ggagacgcca tccacgctgt tttgacctcc atagaagaca | |
| 3661 ccgggaccga tccagcctcc gcggccggga acggtgcatt ggaacgcgga ttccccgtgc | |
| 3721 caagagtgac gtaagtaccg cctatagact ctataggcac acccctttgg ctcttatgca | |
| 3781 tgctatactg tttttggctt ggggcctata cacccccgct tccttatgct ataggtgatg | |
| 3841 gtatagctta gcctataggt gtgggttatt gaccattatt gaccactcca acggtggagg | |
| 3901 gcagtgtagt ctgagcagta ctcgttgctg ccgcgcgcgc caccagacat aatagctgac | |
| 3961 agactaacag actgttcctt tccatgggtc ttttctgcag tcaccgtcgt cgacATGCTG | |
| 4021 CTATCCGTGC CGCTGCTGCT CGGCCTCCTC GGCCTGGCCG TCGCCGAGCC TGCCGTCTAC | |
| 4081 TTCAAGGAGC AGTTTCTGGA CGGGGACGGG TGGACTTCCC GCTGGATCGA ATCCAAACAC | |
| 4141 AAGTCAGATT TTGGCAAATT CGTTCTCAGT TCCGGCAAGT TCTACGGTGA CGAGGAGAAA | |
| 4201 GATAAAGGTT TGCAGACAAG CCAGGATGCA CGCTTTTATG CTCTGTCGGC CAGTTTCGAG | |
| 4261 CCTTTCAGCA ACAAAGGCCA GACGCTGGTG GTGCAGTTCA CGGTGAAACA TGAGCAGAAC | |
| 4321 ATCGACTGTG GGGGCGGCTA TGTGAAGCTG TTTCCTAATA GTTTGGACCA GACAGACATG | |
| 4381 CACGGAGACT CAGAATACAA CATCATGTTT GGTCCCGACA TCTGTGGCCC TGGCACCAAG | |
| 4441 AAGGTTCATG TCATCTTCAA CTACAAGGGC AAGAACGTGC TGATCAACAA GGACATCCGT | |
| 4501 TGCAAGGATG ATGAGTTTAC ACACCTGTAC ACACTGATTG TGCGGCCAGA CAACACCTAT | |
| 4561 GAGGTGAAGA TTGACAACAG CCAGGTGGAG TCCGGCTCCT TGGAAGACGA TTGGGACTTC | |
| 4621 CTGCCACCCA AGAAGATAAA GGATCCTGAT GCTTCAAAAC CGGAAGACTG GGATGAGCGG | |
| 4681 GCCAAGATCG ATGATCCCAC AGACTCCAAG CCTGAGGACT GGGACAAGCC CGAGCATATC | |
| 4741 CCTGACCCTG ATGCTAAGAA GCCCGAGGAC TGGGATGAAG AGATGGACGG AGAGTGGGAA | |
| 4801 CCCCCAGTGA TTCAGAACCC TGAGTACAAG GGTGAGTGGA AGCCCCGGCA GATCGACAAC | |
| 4861 CCAGATTACA AGGGCACTTG GATCCACCCA GAAATTGACA ACCCCGAGTA TTCTCCCGAT | |
| 4921 CCCAGTATCT ATGCCTATGA TAACTTTGGC GTGCTGGGCC TGGACCTCTG GCAGGTCAAG | |
| 4981 TCTGGCACCA TCTTTGACAA CTTCCTCATC ACCAACGATG AGGCATACGC TGAGGAGTTT | |
| 5041 GGCAACGAGA CGTGGGGCGT AACAAAGGCA GCAGAGAAAC AAATGAAGGA CAAACAGGAC | |
| 5101 GAGGAGCAGA GGCTTAAGGA GGAGGAAGAA GACAAGAAAC GCAAAGAGGA GGAGGAGGCA | |
| 5161 GAGGACAAGG AGGATGATGA GGACAAAGAT GAGGATGAGG AGGATGAGGA GGACAAGGAG | |
| 5221 GAAGATGAGG AGGAAGATGT CCCCGGCCAG GCCAAGGACG AGCTG [symbol] [symbol] | |
| [symbol] | |
| [symbol] | |
| [symbol] | |
| [symbol] | |
| [symbol] TAAgg atccagatct | |
| 5581 ttttccctct gccaaaaatt atggggacat catgaagccc cttgagcatc tgacttctgg | |
| 5641 ctaataaagg aaatttattt tcattgcaat agtgtgttgg aattttttgt gtctctcact | |
| 5701 cggaaggaca tatgggaggg caaatcattt aaaacatcag aatgagtatt tggtttagag | |
| 5761 tttggcaaca tatgcccatt cttccgcttc ctcgctcact gactcgctgc gctcggtcgt | |
| 5821 tcggctgcgg cgagcggtat cagctcactc aaaggcggta atacggttat ccacagaatc | |
| 5881 aggggataac gcaggaaaga acatgtgagc aaaaggccag caaaaggcca ggaaccgtaa | |
| 5941 aaaggccgcg ttgctggcgt ttttccatag | 5970 |

| | |
|---|---|
| SEQ ID NO: 33 (coded protein disclosed as SEQ ID NO: 36) | |
| atg acc tct cgc cgc tcc gtg aag tcg ggt ccg cgg gag gtt ccg cgc | 48 |
| Met Thr Ser Arg Arg Ser Val Lys Ser Gly Pro Arg Glu Val Pro Arg | |
| 1 5 10 15 | |
| gat gag tac gag gat ctg tac tac acc ccg tct tca ggt atg gcg agt | 96 |
| Asp Glu Tyr Glu Asp Leu Tyr Tyr Thr Pro Ser Ser Gly Met Ala Ser | |
| 20 25 30 | |
| ccc gat agt ccg cct gac acc tcc cgc cgt ggc gcc cta cag aca cgc | 144 |
| Pro Asp Ser Pro Pro Asp Thr Ser Arg Arg Gly Ala Leu Gln Thr Arg | |
| 35 40 45 | |
| tcg cgc cag agg ggc gag gtc cgt ttc gtc cag tac gac gag tcg gat | 192 |
| Ser Arg Gln Arg Gly Glu Val Arg Phe Val Gln Tyr Asp Glu Ser Asp | |
| 50 55 60 | |
| tat gcc ctc tac ggg ggc tcg tct tcc gaa gac gac gaa cac ccg gag | 240 |
| Tyr Ala Leu Tyr Gly Gly Ser Ser Ser Glu Asp Asp Glu His Pro Glu | |
| 65 70 75 80 | |
| gtc ccc cgg acg cgg cgt ccc gtt tcc ggg gcg gtt ttg tcc ggc ccg | 288 |
| Val Pro Arg Thr Arg Arg Pro Val Ser Gly Ala Val Leu Ser Gly Pro | |
| 85 90 95 | |
| ggg cct gcg cgg gcg cct ccg cca ccc gct ggg tcc gga ggg gcc gga | 336 |
| Gly Pro Ala Arg Ala Pro Pro Pro Pro Ala Gly Ser Gly Gly Ala Gly | |
| 100 105 110 | |
| cgc aca ccc acc acc gcc ccc cgg gcc ccc cga acc cag cgg gtg gcg | 384 |
| Arg Thr Pro Thr Thr Ala Pro Arg Ala Pro Arg Thr Gln Arg Val Ala | |
| 115 120 125 | |
| tct aag gcc ccc gcg gcc ccg gcg gcg gag acc acc cgc ggc agg aaa | 432 |
| Ser Lys Ala Pro Ala Ala Pro Ala Ala Glu Thr Thr Arg Gly Arg Lys | |
| 130 135 140 | |
| tcg gcc cag cca gaa tcc gcc gca ctc cca gac gcc ccc gcg tcg acg | 480 |
| Ser Ala Gln Pro Glu Ser Ala Ala Leu Pro Asp Ala Pro Ala Ser Thr | |
| 145 150 155 160 | |
| gcg cca acc cga tcc aag aca ccc gcg cag ggg ctg gcc aga aag ctg | 528 |
| Ala Pro Thr Arg Ser Lys Thr Pro Ala Gln Gly Leu Ala Arg Lys Leu | |
| 165 170 175 | |
| cac ttt agc acc gcc ccc cca aac ccc gac gcg cca tgg acc ccc cgg | 576 |
| His Phe Ser Thr Ala Pro Pro Asn Pro Asp Ala Pro Trp Thr Pro Arg | |
| 180 185 190 | |
| gtg gcc ggc ttt aac aag cgc gtc ttc tgc gcc gcg gtc ggg cgc ctg | 624 |
| Val Ala Gly Phe Asn Lys Arg Val Phe Cys Ala Ala Val Gly Arg Leu | |
| 195 200 205 | |
| gcg gcc atg cat gcc cgg atg gcg gct gtc cag ctc tgg gac atg tcg | 672 |
| Ala Ala Met His Ala Arg Met Ala Ala Val Gln Leu Trp Asp Met Ser | |
| 210 215 220 | |
| cgt ccg cgc aca gac gaa gac ctc aac gaa ctc ctt ggc atc acc acc | 720 |
| Arg Pro Arg Thr Asp Glu Asp Leu Asn Glu Leu Leu Gly Ile Thr Thr | |
| 225 230 235 240 | |
| atc cgc gtg acg gtc tgc gag ggc aaa aac ctg ctt cag cgc gcc aac | 768 |
| Ile Arg Val Thr Val Cys Glu Gly Lys Asn Leu Leu Gln Arg Ala Asn | |
| 245 250 255 | |
| gag ttg gtg aat cca gac gtg gtg cag gac gtc gac gcg gcc acg gcg | 816 |
| Glu Leu Val Asn Pro Asp Val Val Gln Asp Val Asp Ala Ala Thr Ala | |
| 260 265 270 | |
| act cga ggg cgt tct gcg gcg tcg cgc ccc acc gag cga cct cga gcc | 864 |
| Thr Arg Gly Arg Ser Ala Ala Ser Arg Pro Thr Glu Arg Pro Arg Ala | |
| 275 280 285 | |
| cca gcc cgc tcc gct tct cgc ccc aga cgg ccc gtc gag | 903 |
| Pro Ala Arg Ser Ala Ser Arg Pro Arg Arg Pro Val Glu | |
| 290 295 300 | |
| SEQ ID NO: 34 (coded protein disclosed as SEQ ID NO: 37) | |
| atg acc tct cgc cgc tcc gtg aag tcg ggt ccg cgg gag gtt ccg cgc | 48 |
| Met Thr Ser Arg Arg Ser Val Lys Ser Gly Pro Arg Glu Val Pro Arg | |
| 1 5 10 15 | |
| gat gag tac gag gat ctg tac tac acc ccg tct tca ggt atg gcg agt | 96 |
| Asp Glu Tyr Glu Asp Leu Tyr Tyr Thr Pro Ser Ser Gly Met Ala Ser | |
| 20 25 30 | |
| ccc gat agt ccg cct gac acc tcc cgc cgt ggc gcc cta cag aca cgc | 144 |
| Pro Asp Ser Pro Pro Asp Thr Ser Arg Arg Gly Ala Leu Gln Thr Arg | |
| 35 40 45 | |
| tcg cgc cag agg ggc gag gtc cgt ttc gtc cag tac gac gag tcg gat | 192 |
| Ser Arg Gln Arg Gly Glu Val Arg Phe Val Gln Tyr Asp Glu Ser Asp | |
| 50 55 60 | |
| tat gcc ctc tac ggg ggc tcg tct tcc gaa gac gac gaa cac ccg gag | 240 |
| Tyr Ala Leu Tyr Gly Gly Ser Ser Ser Glu Asp Asp Glu His Pro Glu | |
| 65 70 75 80 | |
| gtc ccc cgg acg cgg cgt ccc gtt tcc ggg gcg gtt ttg tcc ggc ccg | 288 |
| Val Pro Arg Thr Arg Arg Pro Val Ser Gly Ala Val Leu Ser Gly Pro | |
| 85 90 95 | |
| ggg cct gcg cgg gcg cct ccg cca ccc gct ggg tcc gga ggg gcc gga | 336 |
| Gly Pro Ala Arg Ala Pro Pro Pro Pro Ala Gly Ser Gly Gly Ala Gly | |
| 100 105 110 | |
| cgc aca ccc acc acc gcc ccc cgg gcc ccc cga acc cag cgg gtg gcg | 384 |
| Arg Thr Pro Thr Thr Ala Pro Arg Ala Pro Arg Thr Gln Arg Val Ala | |
| 115 120 125 | |
| tct aag gcc ccc gcg gcc ccg gcg gcg gag acc acc cgc ggc agg aaa | 432 |
| Ser Lys Ala Pro Ala Ala Pro Ala Ala Glu Thr Thr Arg Gly Arg Lys | |
| 130 135 140 | |
| tcg gcc cag cca gaa tcc gcc gca ctc cca gac gcc ccc gcg tcg acg | 480 |
| Ser Ala Gln Pro Glu Ser Ala Ala Leu Pro Asp Ala Pro Ala Ser Thr | |
| 145 150 155 160 | |
| gcg cca acc cga tcc aag aca ccc gcg cag ggg ctg gcc aga aag ctg | 528 |
| Ala Pro Thr Arg Ser Lys Thr Pro Ala Gln Gly Leu Ala Arg Lys Leu | |
| 165 170 175 | |
| cac ttt agc acc gcc ccc cca aac ccc gac gcg cca tgg acc ccc cgg | 576 |
| His Phe Ser Thr Ala Pro Pro Asn Pro Asp Ala Pro Trp Thr Pro Arg | |
| 180 185 190 | |
| gtg gcc ggc ttt aac aag cgc gtc ttc tgc gcc gcg gtc ggg cgc ctg | 624 |
| Val Ala Gly Phe Asn Lys Arg Val Phe Cys Ala Ala Val Gly Arg Leu | |
| 195 200 205 | |
| gcg gcc atg cat gcc cgg atg gcg gct gtc cag ctc tgg gac atg tcg | 672 |
| Ala Ala Met His Ala Arg Met Ala Ala Val Gln Leu Trp Asp Met Ser | |
| 210 215 220 | |
| cgt ccg cgc aca gac gaa gac ctc aac gaa ctc ctt ggc atc acc acc | 720 |
| Arg Pro Arg Thr Asp Glu Asp Leu Asn Glu Leu Leu Gly Ile Thr Thr | |
| 225 230 235 240 | |
| atc cgc gtg acg gtc tgc gag ggc aaa aac ctg ctt cag cgc gcc aac | 768 |
| Ile Arg Val Thr Val Cys Glu Gly Lys Asn Leu Leu Gln Arg Ala Asn | |
| 245 250 255 | |
| gag ttg gtg aat cca gac gtg gtg cag gac gtc gac gcg gcc acg gcg | 816 |
| Glu Leu Val Asn Pro Asp Val Val Gln Asp Val Asp Ala Ala Thr Ala | |
| 260 265 270 | |
| act cga ggg cgt tct gcg gcg tcg cgc ccc acc gag cga cct cga gcc | 864 |
| Thr Arg Gly Arg Ser Ala Ala Ser Arg Pro Thr Glu Arg Pro Arg Ala | |
| 275 280 285 | |
| cca gcc cgc tcc gct tct cgc ccc aga cgg ccc gtc gag ggt acc gag | 912 |
| Pro Ala Arg Ser Ala Ser Arg Pro Arg Arg Pro Val Glu Gly Thr Glu | |
| 290 295 300 | |
| ctc gga tcc atg cat gga gat aca cct aca ttg cat gaa tat atg tta | 960 |
| Leu Gly Ser Met His Gly Asp Thr Pro Thr Leu His Glu Tyr Met Leu | |
| 305 310 315 320 | |
| gat ttg caa cca gag aca act gat ctc tac tgt tat gag caa tta aat | 1008 |
| Asp Leu Gln Pro Glu Thr Thr Asp Leu Tyr Cys Tyr Glu Gln Leu Asn | |
| 325 330 335 | |
| gac agc tca gag gag gag gat gaa ata gat ggt cca gct gga caa gca | 1056 |
| Asp Ser Ser Glu Glu Glu Asp Glu Ile Asp Gly Pro Ala Gly Gln Ala | |
| 340 345 350 | |
| gaa ccg gac aga gcc cat tac aat att gta acc ttt tgt tgc aag tgt | 1104 |
| Glu Pro Asp Arg Ala His Tyr Asn Ile Val Thr Phe Cys Cys Lys Cys | |
| 355 360 365 | |
| gac tct acg ctt cgg ttg tgc gta caa agc aca cac gta gac att cgt | 1152 |
| Asp Ser Thr Leu Arg Leu Cys Val Gln Ser Thr His Val Asp Ile Arg | |
| 370 375 380 | |
| act ttg gaa gac ctg tta atg ggc aca cta gga att gtg tgc ccc atc | 1200 |
| Thr Leu Glu Asp Leu Leu Met Gly Thr Leu Gly Ile Val Cys Pro Ile | |
| 385 390 395 400 | |
| tgt tct cag gat aag ctt aag ttt aaa ccg ctg atc agc ctc gac tgt | 1248 |
| Cys Ser Gln Asp Lys Leu Lys Phe Lys Pro Leu Ile Ser Leu Asp Cys | |
| 405 410 415 | |
| gcc ttc tag | 1257 |
| Ala Phe | |
| SEQ ID NO: 35 | |

| | | |
|---|---|---|
| 1 | atg ggg gat tct gaa agg cgg aaa tcg gaa cgg cgt cgt tcc ctt gga | |
| 48 | tat ccc tct gca tat gat gac gtc tcg att cct gct cgc aga cca tca | |
| 96 | aca cgt act cag cga aat tta aac cag gat gat ttg tca aaa cat gga | |
| 144 | cca ttt acc gac cat cca aca caa aaa cat aaa tcg gcg aaa gcc gta | |
| 192 | tcg gaa gac gtt tcg tct acc acc cgg ggt ggc ttt aca aac aaa ccc | |
| 240 | cgt acc aag ccc ggg gtc aga gct gta caa agt aat aaa ttc gct ttc | |
| 288 | agt acg gct cct tca tca gca tct agc act tgg aga tca aat aca gtg | |
| 336 | gca ttt aat cag cgt atg ttt tgc gga gcg gtt gca act gtg gct caa | |
| 384 | tat cac gca tac caa ggc gcg ctc gcc ctt tgg cgt caa gat cct ccg | |
| 432 | cga aca aat gaa gaa tta gat gca ttt ctt tcc aga gct gtc att aaa | |
| 480 | att acc att caa gag ggt cca aat ttg atg ggg gaa gcc gaa acc tgt | |
| 528 | gcc cgc aaa cta ttg gaa gag tct gga tta tcc cag ggg aac gag aac | |
| 576 | gta aag tcc aaa tct gaa cgt aca acc aaa tct gaa cgt aca aga cgc | |
| 624 | ggc ggt gaa att gaa atc aaa tcg cca gat ccg gga tct cat cgt aca | |
| 672 | cat aac cct cgc act ccc gca act tcg cgt cgc cat cat tca tcc gcc | |
| 720 | cgc gga tat cgt agc agt gat agc gaa taa | 747 |

```
SEQ ID NO: 36
Met Thr Ser Arg Arg Ser Val Lys Ser Gly Pro Arg Glu Val Pro Arg
1 5 10 15
Asp Glu Tyr Glu Asp Leu Tyr Tyr Thr Pro Ser Ser Gly Met Ala Ser
                        20 25 30
Pro Asp Ser Pro Pro Asp Thr Ser Arg Arg Gly Ala Leu Gln Thr Arg
                35 40 45
Ser Arg Gln Arg Gly Glu Val Arg Phe Val Gln Tyr Asp Glu Ser Asp
        50 55 60
Tyr Ala Leu Tyr Gly Gly Ser Ser Ser Glu Asp Asp Glu His Pro Glu
65 70 75 80
Val Pro Arg Thr Arg Arg Pro Val Ser Gly Ala Val Leu Ser Gly Pro
                                85 90 95
Gly Pro Ala Arg Ala Pro Pro Pro Pro Ala Gly Ser Gly Gly Ala Gly
                        100 105 110
Arg Thr Pro Thr Thr Ala Pro Arg Ala Pro Arg Thr Gln Arg Val Ala
                115 120 125
Ser Lys Ala Pro Ala Ala Pro Ala Ala Glu Thr Thr Arg Gly Arg Lys
        130 135 140
Ser Ala Gln Pro Glu Ser Ala Ala Leu Pro Asp Ala Pro Ala Ser Thr
145 150 155 160
Ala Pro Thr Arg Ser Lys Thr Pro Ala Gln Gly Leu Ala Arg Lys Leu
                                165 170 175
His Phe Ser Thr Ala Pro Pro Asn Pro Asp Ala Pro Trp Thr Pro Arg
                        180 185 190
Val Ala Gly Phe Asn Lys Arg Val Phe Cys Ala Ala Val Gly Arg Leu
                195 200 205
Ala Ala Met His Ala Arg Met Ala Ala Val Gln Leu Trp Asp Met Ser
        210 215 220
Arg Pro Arg Thr Asp Glu Asp Leu Asn Glu Leu Leu Gly Ile Thr Thr
225 230 235 240
Ile Arg Val Thr Val Cys Glu Gly Lys Asn Leu Leu Gln Arg Ala Asn
                                245 250 255
Glu Leu Val Asn Pro Asp Val Val Gln Asp Val Asp Ala Ala Thr Ala
                        260 265 270
Thr Arg Gly Arg Ser Ala Ala Ser Arg Pro Thr Glu Arg Pro Arg Ala
                275 280 285
Pro Ala Arg Ser Ala Ser Arg Pro Arg Arg Pro Val Glu
        290 295 300
SEQ ID NO: 37
Met Thr Ser Arg Arg Ser Val Lys Ser Gly Pro Arg Glu Val Pro Arg
1 5 10 15
Asp Glu Tyr Glu Asp Leu Tyr Tyr Thr Pro Ser Ser Gly Met Ala Ser
                        20 25 30
Pro Asp Ser Pro Pro Asp Thr Ser Arg Arg Gly Ala Leu Gln Thr Arg
                35 40 45
Ser Arg Gln Arg Gly Glu Val Arg Phe Val Gln Tyr Asp Glu Ser Asp
        50 55 60
Tyr Ala Leu Tyr Gly Gly Ser Ser Ser Glu Asp Asp Glu His Pro Glu
65 70 75 80
Val Pro Arg Thr Arg Arg Pro Val Ser Gly Ala Val Leu Ser Gly Pro
                                85 90 95
Gly Pro Ala Arg Ala Pro Pro Pro Pro Ala Gly Ser Gly Gly Ala Gly
                        100 105 110
Arg Thr Pro Thr Thr Ala Pro Arg Ala Pro Arg Thr Gln Arg Val Ala
                115 120 125
Ser Lys Ala Pro Ala Ala Pro Ala Ala Glu Thr Thr Arg Gly Arg Lys
        130 135 140
Ser Ala Gln Pro Glu Ser Ala Ala Leu Pro Asp Ala Pro Ala Ser Thr
145 150 155 160
Ala Pro Thr Arg Ser Lys Thr Pro Ala Gln Gly Leu Ala Arg Lys Leu
                                165 170 175
His Phe Ser Thr Ala Pro Pro Asn Pro Asp Ala Pro Trp Thr Pro Arg
                        180 185 190
Val Ala Gly Phe Asn Lys Arg Val Phe Cys Ala Ala Val Gly Arg Leu
                195 200 205
Ala Ala Met His Ala Arg Met Ala Ala Val Gln Leu Trp Asp Met Ser
        210 215 220
Arg Pro Arg Thr Asp Glu Asp Leu Asn Glu Leu Leu Gly Ile Thr Thr
225 230 235 240
Ile Arg Val Thr Val Cys Glu Gly Lys Asn Leu Leu Gln Arg Ala Asn
                                245 250 255
Glu Leu Val Asn Pro Asp Val Val Gln Asp Val Asp Ala Ala Thr Ala
                        260 265 270
Thr Arg Gly Arg Ser Ala Ala Ser Arg Pro Thr Glu Arg Pro Arg Ala
                275 280 285
Pro Ala Arg Ser Ala Ser Arg Pro Arg Arg Pro Val Glu Gly Thr Glu
        290 295 300
Leu Gly Ser Met His Gly Asp Thr Pro Thr Leu His Glu Tyr Met Leu
305 310 315 320
Asp Leu Gln Pro Glu Thr Thr Asp Leu Tyr Cys Tyr Glu Gln Leu Asn
                                325 330 335
Asp Ser Ser Glu Glu Glu Asp Glu Ile Asp Gly Pro Ala Gly Gln Ala
                        340 345 350
Glu Pro Asp Arg Ala His Tyr Asn Ile Val Thr Phe Cys Cys Lys Cys
                355 360 365
Asp Ser Thr Leu Arg Leu Cys Val Gln Ser Thr His Val Asp Ile Arg
        370 375 380
Thr Leu Glu Asp Leu Leu Met Gly Thr Leu Gly Ile Val Cys Pro Ile
385 390 395 400
Cys Ser Gln Asp Lys Leu Lys Phe Lys Pro Leu Ile Ser Leu Asp Cys
                                405 410 415
Ala Phe
SEQ ID NO: 38
```

| | | |
|---|---|---|
| 2 | Met Gly Asp Ser Glu Arg Arg Lys Ser Glu Arg Arg Arg Ser Leu Gly | |
| 16 | Tyr Pro Ser Ala Tyr Asp Asp Val Ser Ile Pro Ala Arg Arg Pro Ser | |
| 32 | Thr Arg Thr Gln Arg Asn Leu Asn Gln Asp Asp Leu Ser Lys His Gly | |
| 48 | Pro Phe Thr Asp His Pro Thr Gln Lys His Lys Ser Ala Lys Ala Val | |
| 64 | Ser Glu Asp Val Ser Ser Thr Thr Arg Gly Gly Phe Thr Asn Lys Pro | |
| 80 | Arg Thr Lys Pro Gly Val Arg Ala Val Gln Ser Asn Lys Phe Ala Phe | |
| 96 | Ser Thr Ala Pro Ser Ser Ala Ser Ser Thr Trp Arg Ser Asn Thr Val | |
| 112 | Ala Phe Asn Gln Arg Met Phe Cys Gly Ala Val Ala Thr Val Ala Gln | |
| 128 | Tyr His Ala Tyr Gln Gly Ala Leu Ala Leu Trp Arg Gln Asp Pro Pro | |
| 144 | Arg Thr Asn Glu Glu Leu Asp Ala Phe Leu Ser Arg Ala Val Ile Lys | |
| 160 | Ile Thr Ile Gln Glu Gly Pro Asn Leu Met Gly Glu Ala Glu Thr Cys | |
| 176 | Ala Arg Lys Leu Leu Glu Glu Ser Gly Leu Ser Gln Gly Asn Glu Asn | |
| 192 | Val Lys Ser Lys Ser Glu Arg Thr Thr Lys Ser Glu Arg Thr Arg Arg | |
| 208 | Gly Gly Glu Ile Glu Ile Lys Ser Pro Asp Pro Gly Ser His Arg Thr | |
| 224 | His Asn Pro Arg Thr Pro Ala Thr Ser Arg Arg His His Ser Ser Ala | |
| 240 | Arg Gly Tyr Arg Ser Ser Asp Ser Glu -- | 249 |

| | |
|---|---|
| SEQ ID NO: 39 | |
| Met His Gly Asp Thr Pro Thr Leu His Glu Tyr Met Leu Asp Leu Gln | |
| 1 5 10 15 | |
| Pro Glu Thr Thr Asp Leu Tyr Cys Tyr Glu Gln Leu Asn Asp Ser Ser | |
| 20 25 30 | |
| Glu Glu Glu Asp Glu Ile Asp Gly Pro Ala Gly Gln Ala Glu Pro Asp | |
| 35 40 45 | |
| Arg Ala His Tyr Asn Ile Val Thr Phe Cys Cys Lys Cys Asp Ser Thr | |
| 50 55 60 | |
| Leu Arg Leu Cys Val Gln Ser Thr His Val Asp Ile Arg Thr Leu Glu | |
| 65 70 75 80 | |
| Asp Leu Leu Met Gly Thr Leu Gly Ile Val Cys Pro Ile Cys Ser Gln | |
| 85 90 95 | |
| SEQ ID NO: 40 | |
| gacggatcgg gagatctccc gatcccctat ggtcgactct cagtacaatc tgctctgatg | 60 |
| ccgcatagtt aagccagtat ctgctccctg cttgtgtgtt ggaggtcgct gagtagtgcg | 120 |
| cgagcaaaat ttaagctaca acaaggcaag gcttgaccga caattgcatg aagaatctgc | 180 |
| ttagggttag gcgttttgcg ctgcttcgcg atgtacgggc cagatatacg cgttgacatt | 240 |
| gattattgac tagttattaa tagtaatcaa ttacggggtc attagttcat agcccatata | 300 |
| tggagttccg cgttacataa cttacggtaa atggcccgcc tggctgaccg cccaacgacc | 360 |
| cccgcccatt gacgtcaata atgacgtatg ttcccatagt aacgccaata gggactttcc | 420 |
| attgacgtca atgggtggac tatttacggt aaactgccca cttggcagta catcaagtgt | 480 |
| atcatatgcc aagtacgccc cctattgacg tcaatgacgg taaatggccc gcctggcatt | 540 |
| atgcccagta catgacctta tgggactttc ctacttggca gtacatctac gtattagtca | 600 |
| tcgctattac catggtgatg cggttttggc agtacatcaa tgggcgtgga tagcggtttg | 660 |
| actcacgggg atttccaagt ctccacccca ttgacgtcaa tgggagtttg ttttggcacc | 720 |
| aaaatcaacg ggactttcca aaatgtcgta acaactccgc cccattgacg caaatgggcg | 780 |
| gtaggcgtgt acggtgggag gtctatataa gcagagctct ctggctaact agagaaccca | 840 |
| ctgcttactg gcttatcgaa attaatacga ctcactatag ggagacccaa gctggctagc | 900 |
| gtttaaacgg gccctctaga ctcgagcggc cgccactgtg ctggatatct gcagaattcc | 960 |
| accacactgg actagtggat ccgagctcgg taccaagctt aagtttaaac cgctgatcag | 1020 |
| cctcgactgt gccttctagt tgccagccat ctgttgtttg cccctccccc gtgccttcct | 1080 |
| tgaccctgga aggtgccact cccactgtcc tttcctaata aaatgaggaa attgcatcgc | 1140 |
| attgtctgag taggtgtcat tctattctgg ggggtggggt ggggcaggac agcaaggggg | 1200 |
| aggattggga agacaatagc aggcatgctg gggatgcggt gggctctatg gcttctgagg | 1260 |
| cggaaagaac cagctggggc tctagggggt atccccacgc gccctgtagc ggcgcattaa | 1320 |
| gcgcggcggg tgtggtggtt acgcgcagcg tgaccgctac acttgccagc gccctagcgc | 1380 |
| ccgctccttt cgctttcttc ccttcctttc tcgccacgtt cgccggcttt ccccgtcaag | 1440 |
| ctctaaatcg gggcatccct ttagggttcc gatttagtgc tttacggcac ctcgacccca | 1500 |
| aaaaacttga ttagggtgat ggttcacgta gtgggccatc gccctgatag acggtttttc | 1560 |
| gccctttgac gttggagtcc acgttcttta atagtggact cttgttccaa actggaacaa | 1620 |
| cactcaaccc tatctcggtc tattcttttg atttataagg gattttgggg atttcggcct | 1680 |
| attggttaaa aaatgagctg atttaacaaa aatttaacgc gaattaattc tgtggaatgt | 1740 |
| gtgtcagtta gggtgtggaa agtccccagg ctccccaggc aggcagaagt atgcaaagca | 1800 |
| tgcatctcaa ttagtcagca accaggtgtg gaaagtcccc aggctcccca gcaggcagaa | 1860 |
| gtatgcaaag catgcatctc aattagtcag caaccatagt cccgccccta actccgccca | 1920 |
| tcccgcccct aactccgccc agttccgccc attctccgcc ccatggctga ctaatttttt | 1980 |
| ttatttatgc agaggccgag gccgcctctg cctctgagct attccagaag tagtgaggag | 2040 |
| gcttttttgg aggcctaggc ttttgcaaaa agctcccggg agcttgtata tccattttcg | 2100 |
| gatctgatca agagacagga tgaggatcgt ttcgcatgat tgaacaagat ggattgcacg | 2160 |
| caggttctcc ggccgcttgg gtggagaggc tattcggcta tgactgggca caacagacaa | 2220 |
| tcggctgctc tgatgccgcc gtgttccggc tgtcagcgca ggggcgcccg gttctttttg | 2280 |
| tcaagaccga cctgtccggt gccctgaatg aactgcagga cgaggcagcg cggctatcgt | 2340 |
| ggctggccac gacgggcgtt ccttgcgcag ctgtgctcga cgttgtcact gaagcgggaa | 2400 |
| gggactggct gctattgggc gaagtgccgg ggcaggatct cctgtcatct caccttgctc | 2460 |
| ctgccgagaa agtatccatc atggctgatg caatgcggcg gctgcatacg cttgatccgg | 2520 |
| ctacctgccc attcgaccac caagcgaaac atcgcatcga gcgagcacgt actcggatgg | 2580 |
| aagccggtct tgtcgatcag gatgatctgg acgaagagca tcaggggctc gcgccagccg | 2640 |
| aactgttcgc caggctcaag gcgcgcatgc ccgacggcga ggatctcgtc gtgacccatg | 2700 |
| gcgatgcctg cttgccgaat atcatggtgg aaaatggccg cttttctgga ttcatcgact | 2760 |
| gtggccggct gggtgtggcg gaccgctatc aggacatagc gttggctacc cgtgatattg | 2820 |
| ctgaagagct tggcggcgaa tgggctgacc gcttcctcgt gctttacggt atcgccgctc | 2880 |
| ccgattcgca gcgcatcgcc ttctatcgcc ttcttgacga gttcttctga gcgggactct | 2940 |
| ggggttcgaa atgaccgacc aagcgacgcc caacctgcca tcacgagatt tcgattccac | 3000 |
| cgccgccttc tatgaaaggt tgggcttcgg aatcgttttc cgggacgccg gctggatgat | 3060 |
| cctccagcgc ggggatctca tgctggagtt cttcgcccac cccaacttgt ttattgcagc | 3120 |
| ttataatggt tacaaataaa gcaatagcat cacaaatttc acaaataaag catttttttc | 3180 |
| actgcattct agttgtggtt tgtccaaact catcaatgta tcttatcatg tctgtatacc | 3240 |
| gtcgacctct agctagagct tggcgtaatc atggtcatag ctgtttcctg tgtgaaattg | 3300 |
| ttatccgctc acaattccac acaacatacg agccggaagc ataaagtgta aagcctgggg | 3360 |
| tgcctaatga gtgagctaac tcacattaat tgcgttgcgc tcactgcccg ctttccagtc | 3420 |
| gggaaacctg tcgtgccagc tgcattaatg aatcggccaa cgcgcgggga gaggcggttt | 3480 |
| gcgtattggg cgctcttccg cttcctcgct cactgactcg ctgcgctcgg tcgttcggct | 3540 |
| gcggcgagcg gtatcagctc actcaaaggc ggtaatacgg ttatccacag aatcagggga | 3600 |
| taacgcagga aagaacatgt gagcaaaagg ccagcaaaag gccaggaacc gtaaaaaggc | 3660 |
| cgcgttgctg gcgtttttcc ataggctccg cccccctgac gagcatcaca aaaatcgacg | 3720 |
| ctcaagtcag aggtggcgaa acccgacagg actataaaga taccaggcgt ttccccctgg | 3780 |
| aagctccctc gtgcgctctc ctgttccgac cctgccgctt accggatacc tgtccgcctt | 3840 |
| tctcccttcg ggaagcgtgg cgctttctca atgctcacgc tgtaggtatc tcagttcggt | 3900 |
| gtaggtcgtt cgctccaagc tgggctgtgt gcacgaaccc cccgttcagc ccgaccgctg | 3960 |
| cgccttatcc ggtaactatc gtcttgagtc caacccggta agacacgact tatcgccact | 4020 |
| ggcagcagcc actggtaaca ggattagcag agcgaggtat gtaggcggtg ctacagagtt | 4080 |
| cttgaagtgg tggcctaact acggctacac tagaaggaca gtatttggta tctgcgctct | 4140 |
| gctgaagcca gttaccttcg gaaaaagagt tggtagctct tgatccggca aacaaaccac | 4200 |
| cgctggtagc ggtggttttt ttgtttgcaa gcagcagatt acgcgcagaa aaaaaggatc | 4260 |
| tcaagaagat cctttgatct tttctacggg gtctgacgct cagtggaacg aaaactcacg | 4320 |
| ttaagggatt ttggtcatga gattatcaaa aaggatcttc acctagatcc ttttaaatta | 4380 |
| aaaatgaagt tttaaatcaa tctaaagtat atatgagtaa acttggtctg acagttacca | 4440 |
| atgcttaatc agtgaggcac ctatctcagc gatctgtcta tttcgttcat ccatagttgc | 4500 |
| ctgactcccc gtcgtgtaga taactacgat acgggagggc ttaccatctg gccccagtgc | 4560 |
| tgcaatgata ccgcgagacc cacgctcacc ggctccagat ttatcagcaa taaaccagcc | 4620 |
| agccggaagg gccgagcgca gaagtggtcc tgcaacttta tccgcctcca tccagtctat | 4680 |
| taattgttgc cgggaagcta gagtaagtag ttcgccagtt aatagtttgc gcaacgttgt | 4740 |
| tgccattgct acaggcatcg tggtgtcacg ctcgtcgttt ggtatggctt cattcagctc | 4800 |
| cggttcccaa cgatcaaggc gagttacatg atcccccatg ttgtgcaaaa aagcggttag | 4860 |
| ctccttcggt cctccgatcg ttgtcagaag taagttggcc gcagtgttat cactcatggt | 4920 |
| tatggcagca ctgcataatt ctcttactgt catgccatcc gtaagatgct tttctgtgac | 4980 |
| tggtgagtac tcaaccaagt cattctgaga atagtgtatg cggcgaccga gttgctcttg | 5040 |
| cccggcgtca atacgggata ataccgcgcc acatagcaga actttaaaag tgctcatcat | 5100 |
| tggaaaacgt tcttcggggc gaaaactctc aaggatctta ccgctgttga gatccagttc | 5160 |
| gatgtaaccc actcgtgcac ccaactgatc ttcagcatct tttactttca ccagcgtttc | 5220 |
| tgggtgagca aaaacaggaa ggcaaaatgc cgcaaaaaag ggaataaggg cgacacggaa | 5280 |
| atgttgaata ctcatactct tcctttttca atattattga agcatttatc agggttattg | 5340 |
| tctcatgagc ggatacatat ttgaatgtat ttagaaaaat aaacaaatag gggttccgcg | 5400 |
| cacatttccc cgaaaagtgc cacctgacgt c | 5431 |
| SEQ ID NO: 41 | |
| tggccattgc atacgttgta tccatatcat aatatgtaca tttatattgg ctcatgtcca | 60 |
| acattaccgc catgttgaca ttgattattg actagttatt aatagtaatc aattacgggg | 120 |
| tcattagttc atagcccata tatggagttc cgcgttacat aacttacggt aaatggcccg | 180 |
| cctggctgac cgcccaacga cccccgccca ttgacgtcaa taatgacgta tgttcccata | 240 |
| gtaacgccaa tagggacttt ccattgacgt caatgggtgg agtatttacg gtaaactgcc | 300 |
| cacttggcag tacatcaagt gtatcatatg ccaagtacgc cccctattga cgtcaatgac | 360 |
| ggtaaatggc ccgcctggca ttatgcccag tacatgacct tatgggactt tcctacttgg | 420 |
| cagtacatct acgtattagt catcgctatt accatggtga tgcggttttg gcagtacatc | 480 |
| aatgggcgtg gatagcggtt tgactcacgg ggatttccaa gtctccaccc cattgacgtc | 540 |
| aatgggagtt tgttttggca ccaaaatcaa cgggactttc caaaatgtcg taacaactcc | 600 |
| gccccattga cgcaaatggg cggtaggcgt gtacggtggg aggtctatat aagcagagct | 660 |
| cgtttagtga accgtcagat cgcctggaga cgccatccac gctgttttga cctccataga | 720 |
| agacaccggg accgatccag cctccgcggc cgggaacggt gcattggaac gcggattccc | 780 |
| cgtgccaaga gtgacgtaag taccgcctat agagtctata ggcccacccc cttggcttct | 840 |
| tatgcatgct atactgtttt tggcttgggg tctatacacc cccgcttcct catgttatag | 900 |
| gtgatggtat agcttagcct ataggtgtgg gttattgacc attattgacc actccaacgg | 960 |
| tggagggcag tgtagtctga gcagtactcg ttgctgccgc gcgcgccacc agacataata | 1020 |
| gctgacagac taacagactg ttcctttcca tgggtctttt ctgcagtcac cgtcgtcgac | 1080 |
| ggtatcgata agcttgatat cgaattcacg tgggcccggt accgtatact ctagagcggc | 1140 |
| cgcggatcca gatctttttc cctcgccaaa aattatgggg acatcatgaa gccccttgag | 1200 |
| catctgactt ctggctaata aaggaaattt atttcattgc aatagtgtgt tggaattttt | 1260 |
| tgtgtctctc actcggaagg acatatggga gggcaaatca tttaaaacat cagaatcagt | 1320 |
| atttggttta gagtttggca acatatgcca ttcttccgct tcctcgctca ctgactcgct | 1380 |
| gcgctcggtc gttcggctgc ggcgagcggt atcagctcac tcaaaggcgg taatacggtt | 1440 |
| atccacagaa tcaggggata acgcaggaaa gaacatgtga gcaaaaggcc agcaaaaggc | 1500 |
| caggaaccgt aaaaaggccg cgttgctggc gtttttccat aggctccgcc cccctgacga | 1560 |
| gcatcacaaa aatcgacgct caagtcagag gtggcgaaac ccgacaggac tataaagata | 1620 |
| ccaggcgttt ccccctggaa gctccctcgt gcgctctcct gttccgaccc tgccgcttac | 1680 |
| cggatacctg tccgcctttc tcccttcggg aagcgtggcg ctttctcaat gctcacgctg | 1740 |
| taggtatctc agttcggtgt aggtcgttcg ctccaagctg ggctgtgtgc acgaaccccc | 1800 |
| cgttcagccc gaccgctgcg ccttatccgg taactatcgt cttgagtcca acccggtaag | 1860 |
| acacgactta tcgccactgg cagcagccac tggtaacagg attagcagag cgaggtatgt | 1920 |
| aggcggtgct acagagttct tgaagtggtg gcctaactac ggctacacta gaaggacagt | 1980 |
| atttggtatc tgcgctctgc tgaagccagt taccttcgga aaaagagttg gtagctcttg | 2040 |
| atccggcaaa caaaccaccg ctggtagcgg tggttttttt gtttgcaagc agcagattac | 2100 |
| gcgcagaaaa aaaggatctc aagaagatcc tttgatcttt tctacggggt ctgacgctca | 2160 |
| gtggaacgaa aactcacgtt aagggatttt ggtcatgaga ttatcaaaaa ggatcttcac | 2220 |
| ctagatcctt ttaaattaaa aatgaagttt taaatcaatc taaagtatat atgagtaaac | 2280 |
| ttggtctgac agttaccaat gcttaatcag tgaggcacct atctcagcga tctgtctatt | 2340 |
| tcgttcatcc atagttgcct gactccgggg ggggggggcg ctgaggtctg cctcgtgaag | 2400 |
| aaggtgttgc tgactcatac cagggcaacg ttgttgccat tgctacaggc atcgtggtgt | 2460 |
| cacgctcgtc gtttggtatg gcttcattca gctccggttc ccaacgatca aggcgagtta | 2520 |
| catgatcccc catgttgtgc aaaaaagcgg ttagctcctt cggtcctccg atcgttgtca | 2580 |
| gaagtaagtt ggccgcagtg ttatcactca tggttatggc agcactgcat aattctctta | 2640 |
| ctgtcatgcc atccgtaaga tgcttttctg tgactggtga gtactcaacc aagtcattct | 2700 |
| gagaatagtg tatgcggcga ccgagttgct cttgcccggc gtcaatacgg gataataccg | 2760 |
| cgccacatag cagaacttta aaagtgctca tcattggaaa acgttcttcg gggcgaaaac | 2820 |
| tctcaaggat cttaccgctg ttgagatcca gttcgatgta acccactcgt gcacctgaat | 2880 |
| cgccccatca tccagccaga aagtgaggga gccacggttg atgagagctt tgttgtaggt | 2940 |
| ggaccagttg gtgattttga acttttgctt tgccacggaa cggtctgcgt tgtcgggaag | 3000 |
| atgcgtgatc tgatccttca actcagcaaa agttcgattt attcaacaaa gccgccgtcc | 3060 |
| cgtcaagtca gcgtaatgct ctgccagtgt tacaaccaat taaccaattc tgattagaaa | 3120 |
| aactcatcga gcatcaaatg aaactgcaat ttattcatat caggattatc aataccatat | 3180 |
| ttttgaaaaa gccgtttctg taatgaagga gaaaactcac cgaggcagtt ccataggatg | 3240 |
| gcaagatcct ggtatcggtc tgcgattccg actcgtccaa catcaataca acctattaat | 3300 |
| ttcccctcgt caaaaataag gttatcaagt gagaaatcac catgagtgac gactgaatcc | 3360 |
| ggtgagaatg gcaaaagctt atgcatttct ttccagactt gttcaacagg ccagccatta | 3420 |
| cgctcgtcat caaaatcact cgcatcaacc aaaccgttat tcattcgtga ttgcgcctga | 3480 |
| gcgagacgaa atacgcgatc gctgttaaaa ggacaattac aaacaggaat cgaatgcaac | 3540 |
| cggcgcagga acactgccag cgcatcaaca atattttcac ctgaatcagg atattcttct | 3600 |
| aatacctgga atgctgtttt cccggggatc gcagtggtga gtaaccatgc atcatcagga | 3660 |
| gtacggataa aatgcttgat ggtcggaaga ggcataaatt ccgtcagcca gtttagtctg | 3720 |
| accatctcat ctgtaacatc attggcaacg ctacctttgc catgtttcag aaacaactct | 3780 |
| ggcgcatcgg gcttcccata caatcgatag attgtcgcac ctgattgccc gacattatcg | 3840 |
| cgagcccatt tatacccata taaatcagca tccatgttgg aatttaatcg cggcctcgag | 3900 |
| caagacgttt cccgttgaat atggctcata acaccccttg tattactgtt tatgtaagca | 3960 |
| gacagtttta ttgttcatga tgatatattt ttatcttgtg caatgtaaca tcagagattt | 4020 |
| tgagacacaa cgtggctttc cccccccccc cattattgaa gcatttatca gggttattgt | 4080 |
| ctcatgagcg gatacatatt tgaatgtatt tagaaaaata aacaaatagg ggttccgcgc | 4140 |
| acatttcccc gaaaagtgcc acctgacgtc taagaaacca ttattatcat gacattaacc | 4200 |
| tataaaaata ggcgtatcac gaggcccttt cgtcctcgcg cgtttcggtg atgacggtga | 4260 |
| aaacctctga cacatgcagc tcccggagac ggtcacagct tgtctgtaag cggatgccgg | 4320 |
| gagcagacaa gcccgtcagg gcgcgtcagc gggtgttggc gggtgtcggg gctggcttaa | 4380 |
| ctatgcggca tcagagcaga ttgtactgag agtgcaccat atgcggtgtg aaataccgca | 4440 |
| cagatgcgta aggagaaaat accgcatcag attggctat | 4479 |
| SEQ ID NO: 42 | |
| UGCCUACGAACUCUUCACCdTdT | |
| SEQ ID NO: 43 | |
| GGUGAAGAGUUCGUAGGCAdTdT | |
| SEQ ID NO: 44 | |
| atggcatctggacaaggaccaggtcccccgaaggtgggctgcgatgagtccccgtccccttctga | |
| acagcaggttgcccaggacacagaggaggtctttcgaagctacgttttttacctccaccagcagg | |
| aacaggagacccaggggcggccgcctgccaaccccgagatggacaacttgcccctggaacccaac | |
| agcatcttgggtcaggtgggtcggcagcttgctctcatcggagatgatattaaccggcgctacga | |
| cacagagttccagaatttactagaacagcttcagcccacagccgggaaTGCCTACGAACTCTTCA | |
| CCaagatcgcctccagcctatttaagagtggcatcagctggggccgcgtggtggctctcctgggc | |
| tttggctaccgtctggccctgtacgtctaccagcgtggtttgaccggcttcctgggccaggtgac | |
| ctgctttttggctgatatcatactgcatcattacatcgccagatggatcgcacagagaggcggtt | |
| gggtggcagccctgaatttgcgtagagaccccatcctgaccgtaatggtgatttttggtgtggtt | |
| ctgttgggccaattcgtggtacacagattcttcagatcatga | 637 |
| SEQ ID NO: 45 | |
| TGCCTACGAACTCTTCACC | |
| SEQ ID NO: 46 | |
| UAUGGAGCUGCAGAGGAUGdTdT | |
| SEQ ID NO: 47 | |
| CAUCCUCUGCAGCUCCAUAdTdT | |
| SEQ ID NO: 48 | |
| atggacgggtccggggagcagcttgggagcggcgggcccaccagctctgaacagatcatgaagac | |
| aggggcctttttgctacagggtttcatccaggatcgagcagggaggatggctggggagacacctg | |
| agctgaccttggagcagccgccccaggatgcgtccaccaagaagctgagcgagtgtctccggcga | |
| attggagatgaactggatagcaaTATGGAGCTGCAGAGGATGattgctgacgtggacacggactc | |
| cccccgagaggtcttcttccgggtggcagctgacatgtttgctgatggcaacttcaactggggcc | |
| gcgtggttgccctcttctactttgctagcaaactggtgctcaaggccctgtgcactaaagtgccc | |
| gagctgatcagaaccatcatgggctggacactggacttcctccgtgagcggctgcttgtctggat | |
| ccaagaccagggtggctgggaaggcctcctctcctacttcgggacccccacatggcagacagtga | |
| ccatctttgtggctggagtcctcaccgcctcgctcaccatctggaagaagatgggctga | 589 |
| SEQ ID NO: 49 | |
| TATGGAGCTGCAGAGGATG | |
| SEQ ID NO: 50 | |
| atg gac ttc agc aga aat ctt tat gat att ggg gaa caa ctg gac agt | |
| gaa gat ctg gcc tcc ctc aag ttc ctg agc ctg gac tac att ccg caa | |
| agg aag caa gaa ccc atc aag gat gcc ttg atg tta ttc cag aga ctc | |
| cag gaa aag aga atg ttg gag gaa agc aat ctg tcc ttc ctg aag gag | |
| ctg ctc ttc cga att aat aga ctg gat ttg ctg att acc tac cta aac | |
| act aga aag gag gag atg gaa agg gaa ctt cag aca cca ggc agg gct | |
| caa att tct gcc tac agg ttc cac ttc tgc cgc atg agc tgg gct gaa | |
| gca aac agc cag tgc cag aca cag tct gta cct ttc tgg cgg agg gtc | |
| gat cat cta tta ata agg gtc atg ctc tat cag att tca gaa gaa gtg | |
| agc aga tca gaa ttg agg tct ttt aag ttt ctt ttg caa gag gaa atc | |
| tcc aaa tgc aaa ctg gat gat gac atg aac ctg ctg gat att ttc ata | |
| gag atg gag aag agg gtc atc ctg gga gaa gga aag ttg gac atc ctg | |
| aaa aga gtc tgt gcc caa atc aac aag agc ctg ctg aag ata atc aac | |
| gac tat gaa gaa ttc agc aaa ggg gag gag ttg tgt ggg gta atg aca | |
| atc tcg gac tct cca aga gaa cag gat agt gaa tca cag act ttg gac | |
| aaa gtt tac caa atg aaa agc aaa cct cgg gga tac tgt ctg atc atc | |
| aac aat cac aat ttt gca aaa gca cgg gag aaa gtg ccc aaa ctt cac | |
| agc att agg gac agg aat gga aca cac ttg gat gca ggg gct ttg acc | |
| acg acc ttt gaa gag ctt cat ttt gag atc aag ccc cac gat gac tgc | |
| aca gta gag caa atc tat gag att ttg aaa atc tac caa ctc atg gac | |
| cac agt aac atg gac tgc ttc atc tgc tgt atc ctc tcc cat gga gac | |
| aag ggc atc atc tat ggc act gat gga cag gag gcc ccc atc tat gag | |
| ctg aca tct cag ttc act ggt ttg aag tgc cct tcc ctt gct gga aaa | |
| ccc aaa gtg ttt ttt att cag gct tgt cag ggg gat aac tac cag aaa | |
| ggt ata cct gtt gag act gat tca gag gag caa ccc tat tta gaa atg | |
| gat tta tca tca cct caa acg aga tat atc ccg gat gag gct gac ttt | |
| ctg ctg ggg atg gcc act gtg aat aac tgt gtt tcc tac cga aac cct | |
| gca gag gga acc tgg tac atc cag tca ctt tgc cag agc ctg aga gag | |
| cga tgt cct cga ggc gat gat att ctc acc atc ctg act gaa gtg aac | |
| tat gaa gta agc aac aag gat gac aag aaa aac atg ggg aaa cag atg | |
| cct cag cct act ttc aca cta aga aaa aaa ctt gtc ttc cct tct gat | |
| tga | 1491 |
| SEQ ID NO: 51 | |
| AACCUCGGGGAUACUGUCUGAdTdT | |
| SEQ ID NO: 52 | |
| UCAGACAGUAUCCCCGAGGUUdTdT | |
| SEQ ID NO: 53 | |
| atg gac gaa gcg gat cgg cgg ctc ctg cgg cgg tgc cgg ctg cgg ctg | |
| gtg gaa gag ctg cag gtg gac cag ctc tgg gac gcc ctg ctg agc cgc | |
| gag ctg ttc agg ccc cat atg atc gag gac atc cag cgg gca ggc tct | |
| gga tct cgg cgg gat cag gcc agg cag ctg atc ata gat ctg gag act | |
| cga ggg agt cag gct ctt cct ttg ttc atc tcc tgc tta gag gac aca | |
| ggc cag gac atg ctg gct tcg ttt ctg cga act aac agg caa gca gca | |
| aag ttg tcg aag cca acc cta gaa aac ctt acc cca gtg gtg ctc aga | |
| cca gag att cgc aaa cca gag gtt ctc aga ccg gaa aca ccc aga cca | |
| gtg gac att ggt tct gga gga ttt ggt gat gtc ggt gct ctt gag agt | |
| ttg agg gga aat gca gat ttg gct tac atc ctg agc atg gag ccc tgt | |
| ggc cac tgc ctc att atc aac aat gtg aac ttc tgc cgt gag tcc ggg | |
| ctc cgc acc cgc act ggc tcc aac atc gac tgt gag aag ttg cgg cgt | |
| cgc ttc tcc tcg ctg cat ttc atg gtg gag gtg aag ggc gac ctg act | |
| gcc aag aaa atg gtg ctg gct ttg ctg gag ctg gcg cag cag gac cac | |
| ggt gct ctg gac tgc tgc gtg gtg gtc att ctc tct cac ggc tgt cag | |
| gcc agc cac ctg cag ttc cca ggg gct gtc tac ggc aca gat gga tgc | |
| cct gtg tcg gtc gag aag att gtg aac atc ttc aat ggg acc agc tgc | |
| ccc agc ctg gga ggg aag ccc aag ctc ttt ttc atc cag gcc tgt ggt | |
| ggg gag cag aaa gac cat ggg ttt gag gtg gcc tcc act tcc cct gaa | |
| gac gag tcc cct ggc agt aac ccc gag cca gat gcc acc ccg ttc cag | |
| gaa ggt ttg agg acc ttc gac cag ctg gac gcc ata tct agt ttg ccc | |
| aca ccc agt gac atc ttt gtg tcc tac tct act ttc cca ggt ttt gtt | |
| tcc tgg agg gac ccc aag agt ggc tcc tgg tac gtt gag acc ctg gac | |
| gac atc ttt gag cag tgg gct cac tct gaa gac ctg cag tcc ctc ctg | |
| ctt agg gtc gct aat gct gtt tcg gtg aaa ggg att tat aaa cag atg | |
| cct ggt tgc ttt aat ttc ctc cgg aaa aaa ctt ttc ttt aaa aca tca | |
| taa | 1191 |
| SEQ ID NO: 54 | |
| atg gag aac act gaa aac tca gtg gat tca aaa tcc att aaa aat ttg | |
| gaa cca aag atc ata cat gga agc gaa tca atg gac tct gga ata tcc | |
| ctg gac aac agt tat aaa atg gat tat cct gag atg ggt tta tgt ata | |
| ata att aat aat aag aat ttt cat aaa agc act gga atg aca tct cgg | |
| tct ggt aca gat gtc gat gca gca aac ctc agg gaa aca ttc aga aac | |
| ttg aaa tat gaa gtc agg aat aaa aat gat ctt aca cgt gaa gaa att | |
| gtg gaa ttg atg cgt gat gtt tct aaa gaa gat cac agc aaa agg agc | |
| agt ttt gtt tgt gtg ctt ctg agc cat ggt gaa gaa gga ata att ttt | |
| gga aca aat gga cct gtt gac ctg aaa aaa ata aca aac ttt ttc aga | |
| ggg gat cgt tgt aga agt cta act gga aaa ccc aaa ctt ttc att att | |
| cag gcc tgc cgt ggt aca gaa ctg gac tgt ggc att gag aca gac agt | |
| ggt gtt gat gat gac atg gcg tgt cat aaa ata cca gtg gag gcc gac | |
| ttc ttg tat gca tac tcc aca gca cct ggt tat tat tct tgg cga aat | |
| tca aag gat ggc tcc tgg ttc atc cag tcg ctt tgt gcc atg ctg aaa | |
| cag tat gcc gac aag ctt gaa ttt atg cac att ctt acc cgg gtt aac | |
| cga aag gtg gca aca gaa ttt gag tcc ttt tcc ttt gac gct act ttt | |
| cat gca aag aaa cag att cca tgt att gtt tcc atg ctc aca aaa gaa | |
| ctc tat ttt tat cac taa | 834 |
| SEQ ID NO: 55 | |
| atggcgtacc catacgatgt tccagattac gctagcttga gatctaccat gtctcagagc | 60 |
| aaccgggagc tggtggttga ctttctctcc tacaagcttt cccagaaagg atacagctgg | 120 |
| agtcagttta gtgatgtgga agagaacagg actgaggccc cagaagggac tgaatcggag | 180 |
| atggagaccc ccagtgccat caatggcaac ccatcctggc acctggcaga cagccccgcg | 240 |
| gtgaatggag ccactgcgca cagcagcagt ttggatgccc gggaggtgat ccccatggca | 300 |
| gcagtaaagc aagcgctgag ggaggcaggc gacgagtttg aactgcggta ccggcgggca | 360 |
| ttcagtgacc tgacatccca gctccacatc accccaggga cagcatatca gagctttgaa | 420 |
| caggtagtga atgaactctt ccgggatggg gtaaactggg gtcgcattgt ggcctttttc | 480 |
| tccttcggcg gggcactgtg cgtggaaagc gtagacaagg agatgcaggt attggtgagt | 540 |
| cggatcgcag cttggatggc cacttacctg aatgaccacc tagagccttg gatccaggag | 600 |
| aacggcggct gggatacttt tgtggaactc tatgggaaca atgcagcagc cgagagccga | 660 |
| aagggccagg aacgcttcaa ccgctggttc ctgacgggca tgactgtggc cggcgtggtt | 720 |
| ctgctgggct cactcttcag tcggaaatga | 750 |
| SEQ ID NO: 56 | |
| Met Ala Tyr Pro Tyr Asp Val Pro Asp Tyr Ala Ser Leu Arg Ser Thr | |
| 1 5 10 15 | |
| Met Ser Gln Ser Asn Arg Glu Leu Val Val Asp Phe Leu Ser Tyr Lys | |
| 20 25 30 | |
| Leu Ser Gln Lys Gly Tyr Ser Trp Ser Gln Phe Ser Asp Val Glu Glu | |
| 35 40 45 | |
| Asn Arg Thr Glu Ala Pro Glu Gly Thr Glu Ser Glu Met Glu Thr Pro | |
| 50 55 60 | |
| Ser Ala Ile Asn Gly Asn Pro Ser Trp His Leu Ala Asp Ser Pro Ala | |
| 65 70 75 80 | |
| Val Asn Gly Ala Thr Ala His Ser Ser Ser Leu Asp Ala Arg Glu Val | |
| 85 90 95 | |
| Ile Pro Met Ala Ala Val Lys Gln Ala Leu Arg Glu Ala Gly Asp Glu | |
| 100 105 110 | |
| Phe Glu Leu Arg Tyr Arg Arg Ala Phe Ser Asp Leu Thr Ser Gln Leu | |
| 115 120 125 | |
| His Ile Thr Pro Gly Thr Ala Tyr Gln Ser Phe Glu Gln Val Val Asn | |
| 130 135 140 | |
| Glu Leu Phe Arg Asp Gly Val Asn Trp Gly Arg Ile Val Ala Phe Phe | |
| 145 150 155 160 | |
| Ser Phe Gly Gly Ala Leu Cys Val Glu Ser Val Asp Lys Glu Met Gln | |
| 165 170 175 | |
| Val Leu Val Ser Arg Ile Ala Ala Trp Met Ala Thr Tyr Leu Asn Asp | |
| 180 185 190 | |
| His Leu Glu Pro Trp Ile Gln Glu Asn Gly Gly Trp Asp Thr Phe Val | |
| 195 200 205 | |
| Glu Leu Tyr Gly Asn Asn Ala Ala Ala Glu Ser Arg Lys Gly Gln Glu | |
| 210 215 220 | |
| Arg Phe Asn Arg Trp Phe Leu Thr Gly Met Thr Val Ala Gly Val Val | |
| 225 230 235 240 | |
| Leu Leu Gly Ser Leu Phe Ser Arg Lys | |
| 245 | |
| SEQ ID NO: 57 | |
| gacggatcgg gagatctccc gatcccctat ggtcgactct cagtacaatc tgctctgatg | 60 |
| ccgcatagtt aagccagtat ctgctccctg cttgtgtgtt ggaggtcgct gagtagtgcg | 120 |
| cgagcaaaat ttaagctaca acaaggcaag gcttgaccga caattgcatg aagaatctgc | 180 |
| ttagggttag gcgttttgcg ctgcttcgcg atgtacgggc cagatatacg cgttgacatt | 240 |
| gattattgac tagttattaa tagtaatcaa ttacggggtc attagttcat agcccatata | 300 |
| tggagttccg cgttacataa cttacggtaa atggcccgcc tggctgaccg cccaacgacc | 360 |
| cccgcccatt gacgtcaata atgacgtatg ttcccatagt aacgccaata gggactttcc | 420 |
| attgacgtca atgggtggac tatttacggt aaactgccca cttggcagta catcaagtgt | 480 |
| atcatatgcc aagtacgccc cctattgacg tcaatgacgg taaatggccc gcctggcatt | 540 |
| atgcccagta catgacctta tgggactttc ctacttggca gtacatctac gtattagtca | 600 |
| tcgctattac catggtgatg cggttttggc agtacatcaa tgggcgtgga tagcggtttg | 660 |
| actcacgggg atttccaagt ctccacccca ttgacgtcaa tgggagtttg ttttggcacc | 720 |
| aaaatcaacg ggactttcca aaatgtcgta acaactccgc cccattgacg caaatgggcg | 780 |
| gtaggcgtgt acggtgggag gtctatataa gcagagctct ctggctaact agagaaccca | 840 |
| ctgcttactg gcttatcgaa attaatacga ctcactatag ggagacccaa gctggctagc | 900 |
| gtttaaacgg gccctctaga ctcgagcggc cgccactgtg ctggatatct gcagaattcc | 960 |
| accacactgg actagtggat ctatggcgta cccatacgat gttccagatt acgctagctt | 1020 |
| gagatctacc atgtctcaga gcaaccggga gctggtggtt gactttctct cctacaagct | 1080 |
| ttcccagaaa ggatacagct ggagtcagtt tagtgatgtg gaagagaaca ggactgaggc | 1140 |
| cccagaaggg actgaatcgg agatggagac ccccagtgcc atcaatggca acccatcctg | 1200 |
| gcacctggca gacagccccg cggtgaatgg agccactgcg cacagcagca gtttggatgc | 1260 |
| ccgggaggtg atccccatgg cagcagtaaa gcaagcgctg agggaggcag gcgacgagtt | 1320 |
| tgaactgcgg taccggcggg cattcagtga cctgacatcc cagctccaca tcaccccagg | 1380 |
| gacagcatat cagagctttg aacaggtagt gaatgaactc ttccgggatg gggtaaactg | 1440 |
| gggtcgcatt gtggcctttt tctccttcgg cggggcactg tgcgtggaaa gcgtagacaa | 1500 |
| ggagatgcag gtattggtga gtcggatcgc agcttggatg gccacttacc tgaatgacca | 1560 |
| cctagagcct tggatccagg agaacggcgg ctgggatact tttgtggaac tctatgggaa | 1620 |
| caatgcagca gccgagagcc gaaagggcca ggaacgcttc aaccgctggt tcctgacggg | 1680 |
| catgactgtg gccggcgtgg ttctgctggg ctcactcttc agtcggaaat gaagatccga | 1740 |
| gctcggtacc aagcttaagt ttaaaccgct gatcagcctc gactgtgcct tctagttgcc | 1800 |
| agccatctgt tgtttgcccc tcccccgtgc cttccttgac cctggaaggt gccactccca | 1860 |
| ctgtcctttc ctaataaaat gaggaaaatg catcgcattg tctgagtagg tgtcattcta | 1920 |
| ttctgggggg tggggtgggg caggacagca agggggagga ttgggaagac aatagcaggc | 1980 |
| atgctgggga tgcggtgggc tctatggctt ctgaggcgga aagaaccagc tggggctcta | 2040 |
| gggggtatcc ccacgcgccc tgtagcggcg cattaagcgc ggcgggtgtg gtggttacgc | 2100 |
| gcagcgtgac cgctacactt gccagcgccc tagcgcccgc tcctttcgct ttcttccctt | 2160 |
| cctttctcgc cacgttcgcc ggctttcccc gtcaagctct aaatcggggc atccctttag | 2220 |
| ggttccgatt tagtgcttta cggcacctcg accccaaaaa acttgattag ggtgatggtt | 2280 |
| cacgtagtgg gccatcgccc tgatagacgg tttttcgccc tttgacgttg gagtccacgt | 2340 |
| tctttaatag tggactcttg ttccaaactg gaacaacact caaccctatc tcggtctatt | 2400 |
| cttttgattt ataagggatt ttggggattt cggcctattg gttaaaaaat gagctgattt | 2460 |
| aacaaaaatt taacgcgaat taattctgtg gaatgtgtgt cagttagggt gtggaaagtc | 2520 |
| cccaggctcc ccaggcaggc agaagtatgc aaagcatgca tctcaattag tcagcaacca | 2580 |
| ggtgtggaaa gtccccaggc tccccagcag gcagaagtat gcaaagcatg catctcaatt | 2640 |
| agtcagcaac catagtcccg cccctaactc cgcccatccc gcccctaact ccgcccagtt | 2700 |
| ccgcccattc tccgccccat ggctgactaa ttttttttat ttatgcagag gccgaggccg | 2760 |
| cctctgcctc tgagctattc cagaagtagt gaggaggctt ttttggaggc ctaggctttt | 2820 |
| gcaaaaagct cccgggagct tgtatatcca ttttcggatc tgatcaagag acaggatgag | 2880 |
| gatcgtttcg catgattgaa caagatggat tgcacgcagg ttctccggcc gcttgggtgg | 2940 |
| agaggctatt cggctatgac tgggcacaac agacaatcgg ctgctctgat gccgccgtgt | 3000 |
| tccggctgtc agcgcagggg cgcccggttc tttttgtcaa gaccgacctg tccggtgccc | 3060 |
| tgaatgaact gcaggacgag gcagcgcggc tatcgtggct ggccacgacg ggcgttcctt | 3120 |
| gcgcagctgt gctcgacgtt gtcactgaag cgggaaggga ctggctgcta ttgggcgaag | 3180 |
| tgccggggca ggatctcctg tcatctcacc ttgctcctgc cgagaaagta tccatcatgg | 3240 |
| ctgatgcaat gcggcggctg catacgcttg atccggctac ctgcccattc gaccaccaag | 3300 |
| cgaaacatcg catcgagcga gcacgtactc ggatggaagc cggtcttgtc gatcaggatg | 3360 |
| atctggacga agagcatcag gggctcgcgc cagccgaact gttcgccagg ctcaaggcgc | 3420 |
| gcatgcccga cggcgaggat ctcgtcgtga cccatggcga tgcctgcttg ccgaatatca | 3480 |
| tggtggaaaa tggccgcttt tctggattca tcgactgtgg ccggctgggt gtggcggacc | 3540 |
| gctatcagga catagcgttg gctacccgtg atattgctga agagcttggc ggcgaatggg | 3600 |
| ctgaccgctt cctcgtgctt tacggtatcg ccgctcccga ttcgcagcgc atcgccttct | 3660 |
| atcgccttct tgacgagttc ttctgagcgg gactctgggg ttcgaaatga ccgaccaagc | 3720 |
| gacgcccaac ctgccatcac gagatttcga ttccaccgcc gccttctatg aaaggttggg | 3780 |
| cttcggaatc gttttccggg acgccggctg gatgatcctc cagcgcgggg atctcatgct | 3840 |
| ggagttcttc gcccacccca acttgtttat tgcagcttat aatggttaca aataaagcaa | 3900 |
| tagcatcaca aatttcacaa ataaagcatt tttttcactg cattctagtt gtggtttgtc | 3960 |
| caaactcatc aatgtatctt atcatgtctg tataccgtcg acctctagct agagcttggc | 4020 |
| gtaatcatgg tcatagctgt ttcctgtgtg aaattgttat ccgctcacaa ttccacacaa | 4080 |
| catacgagcc ggaagcataa agtgtaaagc ctggggtgcc taatgagtga gctaactcac | 4140 |
| attaattgcg ttgcgctcac tgcccgcttt ccagtcggga aacctgtcgt gccagctgca | 4200 |
| ttaatgaatc ggccaacgcg cggggagagg cggtttgcgt attgggcgct cttccgcttc | 4260 |
| ctcgctcact gactcgctgc gctcggtcgt tcggctgcgg cgagcggtat cagctcactc | 4320 |
| aaaggcggta atacggttat ccacagaatc aggggataac gcaggaaaga acatgtgagc | 4380 |
| aaaaggccag caaaaggcca ggaaccgtaa aaaggccgcg ttgctggcgt ttttccatag | 4440 |
| gctccgcccc cctgacgagc atcacaaaaa tcgacgctca agtcagaggt ggcgaaaccc | 4500 |
| gacaggacta taaagatacc aggcgtttcc ccctggaagc tccctcgtgc gctctcctgt | 4560 |
| tccgaccctg ccgcttaccg gatacctgtc cgcctttctc ccttcgggaa gcgtggcgct | 4620 |
| ttctcaatgc tcacgctgta ggtatctcag ttcggtgtag gtcgttcgct ccaagctggg | 4680 |
| ctgtgtgcac gaaccccccg ttcagcccga ccgctgcgcc ttatccggta actatcgtct | 4740 |
| tgagtccaac ccggtaagac acgacttatc gccactggca gcagccactg gtaacaggat | 4800 |
| tagcagagcg aggtatgtag gcggtgctac agagttcttg aagtggtggc ctaactacgg | 4860 |
| ctacactaga aggacagtat ttggtatctg cgctctgctg aagccagtta ccttcggaaa | 4920 |
| aagagttggt agctcttgat ccggcaaaca aaccaccgct ggtagcggtg gtttttttgt | 4980 |
| ttgcaagcag cagattacgc gcagaaaaaa aggatctcaa gaagatcctt tgatcttttc | 5040 |
| tacggggtct gacgctcagt ggaacgaaaa ctcacgttaa gggattttgg tcatgagatt | 5100 |
| atcaaaaagg atcttcacct agatcctttt aaattaaaaa tgaagtttta aatcaatcta | 5160 |
| aagtatatat gagtaaactt ggtctgacag ttaccaatgc ttaatcagtg aggcacctat | 5220 |
| ctcagcgatc tgtctatttc gttcatccat agttgcctga ctccccgtcg tgtagataac | 5280 |
| tacgatacgg gagggcttac catctggccc cagtgctgca atgataccgc gagacccacg | 5340 |
| ctcaccggct ccagatttat cagcaataaa ccagccagcc ggaagggccg agcgcagaag | 5400 |
| tggtcctgca actttatccg cctccatcca gtctattaat tgttgccggg aagctagagt | 5460 |
| aagtagttcg ccagttaata gtttgcgcaa cgttgttgcc attgctacag gcatcgtggt | 5520 |
| gtcacgctcg tcgtttggta tggcttcatt cagctccggt tcccaacgat caaggcgagt | 5580 |
| tacatgatcc cccatgttgt gcaaaaaagc ggttagctcc ttcggtcctc cgatcgttgt | 5640 |
| cagaagtaag ttggccgcag tgttatcact catggttatg gcagcactgc ataattctct | 5700 |
| tactgtcatg ccatccgtaa gatgcttttc tgtgactggt gagtactcaa ccaagtcatt | 5760 |
| ctgagaatag tgtatgcggc gaccgagttg ctcttgcccg gcgtcaatac gggataatac | 5820 |
| cgcgccacat agcagaactt taaaagtgct catcattgga aaacgttctt cggggcgaaa | 5880 |
| actctcaagg atcttaccgc tgttgagatc cagttcgatg taacccactc gtgcacccaa | 5940 |
| ctgatcttca gcatctttta ctttcaccag cgtttctggg tgagcaaaaa caggaaggca | 6000 |
| aaatgccgca aaaaagggaa taagggcgac acggaaatgt tgaatactca tactcttcct | 6060 |
| ttttcaatat tattgaagca tttatcaggg ttattgtctc atgagcggat acatatttga | 6120 |
| atgtatttag aaaaataaac aaataggggt tccgcgcaca tttccccgaa aagtgccacc | 6180 |
| tgacgtc | 6187 |
| SEQ ID NO: 58 | |
| gacggatcgg gagatctccc gatcccctat ggtcgactct cagtacaatc tgctctgatg | 60 |
| ccgcatagtt aagccagtat ctgctccctg cttgtgtgtt ggaggtcgct gagtagtgcg | 120 |
| cgagcaaaat ttaagctaca acaaggcaag gcttgaccga caattgcatg aagaatctgc | 180 |
| ttagggttag gcgttttgcg ctgcttcgcg atgtacgggc cagatatacg cgttgacatt | 240 |
| gattattgac tagttattaa tagtaatcaa ttacggggtc attagttcat agcccatata | 300 |
| tggagttccg cgttacataa cttacggtaa atggcccgcc tggctgaccg cccaacgacc | 360 |
| cccgcccatt gacgtcaata atgacgtatg ttcccatagt aacgccaata gggactttcc | 420 |
| attgacgtca atgggtggac tatttacggt aaactgccca cttggcagta catcaagtgt | 480 |
| atcatatgcc aagtacgccc cctattgacg tcaatgacgg taaatggccc gcctggcatt | 540 |
| atgcccagta catgacctta tgggactttc ctacttggca gtacatctac gtattagtca | 600 |
| tcgctattac catggtgatg cggttttggc agtacatcaa tgggcgtgga tagcggtttg | 660 |
| actcacgggg atttccaagt ctccacccca ttgacgtcaa tgggagtttg ttttggcacc | 720 |
| aaaatcaacg ggactttcca aaatgtcgta acaactccgc cccattgacg caaatgggcg | 780 |
| gtaggcgtgt acggtgggag gtctatataa gcagagctct ctggctaact agagaaccca | 840 |
| ctgcttactg gcttatcgaa attaatacga ctcactatag ggagacccaa gctggctagc | 900 |
| gtttaaacgg gccctctaga ctcgagcggc cgccactgtg ctggatatct gcagaattca | 960 |
| tgcatggaga tacacctaca ttgcatgaat atatgttaga tttgcaacca gagacaactg | 1020 |
| atctctactg ttatgagcaa ttaaatgaca gctcagagga ggaggatgaa atagatggtc | 1080 |
| cagctggaca agcagaaccg gacagagccc attacaatat tgtaaccttt tgttgcaagt | 1140 |
| gtgactctac gcttcggttg tgcgtacaaa gcacacacgt agacattcgt actttggaag | 1200 |
| acctgttaat gggcacacta ggaattgtgt gccccatctg ttctcagaaa ccaggatcta | 1260 |
| tggcgtaccc atacgatgtt ccagattacg ctagcttgag atctaccatg tctcagagca | 1320 |
| accgggagct ggtggttgac tttctctcct acaagctttc ccagaaagga tacagctgga | 1380 |
| gtcagtttag tgatgtggaa gagaacagga ctgaggcccc agaagggact gaatcggaga | 1440 |
| tggagacccc cagtgccatc aatggcaacc catcctggca cctggcagac agccccgcgg | 1500 |
| tgaatggagc cactgcgcac agcagcagtt tggatgcccg ggaggtgatc cccatggcag | 1560 |
| cagtaaagca agcgctgagg gaggcaggcg acgagtttga actgcggtac cggcgggcat | 1620 |
| tcagtgacct gacatcccag ctccacatca ccccagggac agcatatcag agctttgaac | 1680 |
| aggtagtgaa tgaactcttc cgggatgggg taaactgggg tcgcattgtg gcctttttct | 1740 |
| ccttcggcgg ggcactgtgc gtggaaagcg tagacaagga gatgcaggta ttggtgagtc | 1800 |
| ggatcgcagc ttggatggcc acttacctga atgaccacct agagccttgg atccaggaga | 1860 |
| acggcggctg ggatactttt gtggaactct atgggaacaa tgcagcagcc gagagccgaa | 1920 |
| agggccagga acgcttcaac cgctggttcc tgacgggcat gactgtggcc ggcgtggttc | 1980 |
| tactgggctc actcttcagt cggaaatgaa gatccaagct taagtttaaa ccgctgatca | 2040 |
| gcctcgactg tgccttctag ttgccagcca tctgttgttt gcccctcccc cgtgccttcc | 2100 |
| ttgaccctgg aaggtgccac tcccactgtc ctttcctaat aaaatgagga aattgcatcg | 2160 |
| cattgtctga gtaggtgtca ttctattctg gggggtgggg tggggcagga cagcaagggg | 2220 |
| gaggattggg aagacaatag caggcatgct ggggatgcgg tgggctctat ggcttctgag | 2280 |
| gcggaaagaa ccagctgggg ctctaggggg tatccccacg cgccctgtag cggcgcatta | 2340 |
| agcgcggcgg gtgtggtggt tacgcgcagc gtgaccgcta cacttgccag cgccctagcg | 2400 |
| cccgctcctt tcgctttctt cccttccttt ctcgccacgt tcgccggctt tccccgtcaa | 2460 |
| gctctaaatc ggggcatccc tttagggttc cgatttagtg ctttacggca cctcgacccc | 2520 |
| aaaaaacttg attagggtga tggttcacgt agtgggccat cgccctgata gacggttttt | 2580 |
| cgccctttga cgttggagtc cacgttcttt aatagtggac tcttgttcca aactggaaca | 2640 |
| acactcaacc ctatctcggt ctattctttt gatttataag ggattttggg gatttcggcc | 2700 |
| tattggttaa aaaatgagct gatttaacaa aaatttaacg cgaattaatt ctgtggaatg | 2760 |
| tgtgtcagtt agggtgtgga aagtccccag gctccccagg caggcagaag tatgcaaagc | 2820 |
| atgcatctca attagtcagc aaccaggtgt ggaaagtccc caggctcccc agcaggcaga | 2880 |
| agtatgcaaa gcatgcatct caattagtca gcaaccatag tcccgcccct aactccgccc | 2940 |
| atcccgcccc taactccgcc cagttccgcc cattctccgc cccatggctg actaattttt | 3000 |
| tttatttatg cagaggccga ggccgcctct gcctctgagc tattccagaa gtagtgagga | 3060 |
| ggcttttttg gaggcctagg cttttgcaaa aagctcccgg gagcttgtat atccattttc | 3120 |
| ggatctgatc aagagacagg atgaggatcg tttcgcatga ttgaacaaga tggattgcac | 3180 |
| gcaggttctc cggccgcttg ggtggagagg ctattcggct atgactgggc acaacagaca | 3240 |
| atcggctgct ctgatgccgc cgtgttccgg ctgtcagcgc aggggcgccc ggttcttttt | 3300 |
| gtcaagaccg acctgtccgg tgccctgaat gaactgcagg acgaggcagc gcggctatcg | 3360 |
| tggctggcca cgacgggcgt tccttgcgca gctgtgctcg acgttgtcac tgaagcggga | 3420 |
| agggactggc tgctattggg cgaagtgccg gggcaggatc tcctgtcatc tcaccttgct | 3480 |
| cctgccgaga aagtatccat catggctgat gcaatgcggc ggctgcatac gcttgatccg | 3540 |
| gctacctgcc cattcgacca ccaagcgaaa catcgcatcg agcgagcacg tactcggatg | 3600 |
| gaagccggtc ttgtcgatca ggatgatctg gacgaagagc atcaggggct cgcgccagcc | 3660 |
| gaactgttcg ccaggctcaa ggcgcgcatg cccgacggcg aggatctcgt cgtgacccat | 3720 |
| ggcgatgcct gcttgccgaa tatcatggtg gaaaatggcc gcttttctgg attcatcgac | 3780 |
| tgtggccggc tgggtgtggc ggaccgctat caggacatag cgttggctac ccgtgatatt | 3840 |
| gctgaagagc ttggcggcga atgggctgac cgcttcctcg tgctttacgg tatcgccgct | 3900 |
| cccgattcgc agcgcatcgc cttctatcgc cttcttgacg agttcttctg agcgggactc | 3960 |
| tggggttcga aatgaccgac caagcgacgc ccaacctgcc atcacgagat ttcgattcca | 4020 |
| ccgccgcctt ctatgaaagg ttgggcttcg gaatcgtttt ccgggacgcc ggctggatga | 4080 |
| tcctccagcg cggggatctc atgctggagt tcttcgccca ccccaacttg tttattgcag | 4140 |
| cttataatgg ttacaaataa agcaatagca tcacaaattt cacaaataaa gcattttttt | 4200 |
| cactgcattc tagttgtggt ttgtccaaac tcatcaatgt atcttatcat gtctgtatac | 4260 |
| cgtcgacctc tagctagagc ttggcgtaat catggtcata gctgtttcct gtgtgaaatt | 4320 |
| gttatccgct cacaattcca cacaacatac gagccggaag cataaagtgt aaagcctggg | 4380 |
| gtgcctaatg agtgagctaa ctcacattaa ttgcgttgcg ctcactgccc gctttccagt | 4440 |
| cgggaaacct gtcgtgccag ctgcattaat gaatcggcca acgcgcgggg agaggcggtt | 4500 |
| tgcgtattgg gcgctcttcc gcttcctcgc tcactgactc gctgcgctcg gtcgttcggc | 4560 |
| tgcggcgagc ggtatcagct cactcaaagg cggtaatacg gttatccaca gaatcagggg | 4620 |
| ataacgcagg aaagaacatg tgagcaaaag gccagcaaaa ggccaggaac cgtaaaaagg | 4680 |
| ccgcgttgct ggcgtttttc cataggctcc gcccccctga cgagcatcac aaaaatcgac | 4740 |
| gctcaagtca gaggtggcga aacccgacag gactataaag ataccaggcg tttccccctg | 4800 |
| gaagctccct cgtgcgctct cctgttccga ccctgccgct taccggatac ctgtccgcct | 4860 |
| ttctcccttc gggaagcgtg gcgctttctc aatgctcacg ctgtaggtat ctcagttcgg | 4920 |
| tgtaggtcgt tcgctccaag ctgggctgtg tgcacgaacc ccccgttcag cccgaccgct | 4980 |
| gcgccttatc cggtaactat cgtcttgagt ccaacccggt aagacacgac ttatcgccac | 5040 |
| tggcagcagc cactggtaac aggattagca gagcgaggta tgtaggcggt gctacagagt | 5100 |
| tcttgaagtg gtggcctaac tacggctaca ctagaaggac agtatttggt atctgcgctc | 5160 |
| tgctgaagcc agttaccttc ggaaaaagag ttggtagctc ttgatccggc aaacaaacca | 5220 |
| ccgctggtag cggtggtttt tttgtttgca agcagcagat tacgcgcaga aaaaaaggat | 5280 |
| ctcaagaaga tcctttgatc ttttctacgg ggtctgacgc tcagtggaac gaaaactcac | 5340 |
| gttaagggat tttggtcatg agattatcaa aaaggatctt cacctagatc cttttaaatt | 5400 |
| aaaaatgaag ttttaaatca atctaaagta tatatgagta aacttggtct gacagttacc | 5460 |
| aatgcttaat cagtgaggca cctatctcag cgatctgtct atttcgttca tccatagttg | 5520 |
| cctgactccc cgtcgtgtag ataactacga tacgggaggg cttaccatct ggccccagtg | 5580 |
| ctgcaatgat accgcgagac ccacgctcac cggctccaga tttatcagca ataaaccagc | 5640 |
| cagccggaag ggccgagcgc agaagtggtc ctgcaacttt atccgcctcc atccagtcta | 5700 |
| ttaattgttg ccgggaagct agagtaagta gttcgccagt taatagtttg cgcaacgttg | 5760 |
| ttgccattgc tacaggcatc gtggtgtcac gctcgtcgtt tggtatggct tcattcagct | 5820 |
| ccggttccca acgatcaagg cgagttacat gatcccccat gttgtgcaaa aaagcggtta | 5880 |
| gctccttcgg tcctccgatc gttgtcagaa gtaagttggc cgcagtgtta tcactcatgg | 5940 |
| ttatggcagc actgcataat tctcttactg tcatgccatc cgtaagatgc ttttctgtga | 6000 |
| ctggtgagta ctcaaccaag tcattctgag aatagtgtat gcggcgaccg agttgctctt | 6060 |
| gcccggcgtc aatacgggat aataccgcgc cacatagcag aactttaaaa gtgctcatca | 6120 |
| ttggaaaacg ttcttcgggg cgaaaactct caaggatctt accgctgttg agatccagtt | 6180 |
| cgatgtaacc cactcgtgca cccaactgat cttcagcatc ttttactttc accagcgttt | 6240 |
| ctgggtgagc aaaaacagga aggcaaaatg ccgcaaaaaa gggaataagg gcgacacgga | 6300 |
| aatgttgaat actcatactc ttcctttttc aatattattg aagcatttat cagggttatt | 6360 |
| gtctcatgag cggatacata tttgaatgta tttagaaaaa taaacaaata ggggttccgc | 6420 |
| gcacatttcc ccgaaaagtg ccacctgacg tc | 6452 |
| SEQ ID NO: 59 | |
| Met His Gly Asp Thr Pro Thr Leu His Glu Tyr Met Leu Asp Leu Gln | |
| 1 5 10 15 | |
| Pro Glu Thr Thr Asp Leu Tyr Cys Tyr Glu Gln Leu Asn Asp Ser Ser | |
| 20 25 30 | |
| Glu Glu Glu Asp Glu Ile Asp Gly Pro Ala Gly Gln Ala Glu Pro Asp | |
| 35 40 45 | |
| Arg Ala His Tyr Asn Ile Val Thr Phe Cys Cys Lys Cys Asp Ser Thr | |
| 50 55 60 | |
| Leu Arg Leu Cys Val Gln Ser Thr His Val Asp Ile Arg Thr Leu Glu | |
| 65 70 75 80 | |
| Asp Leu Leu Met Gly Thr Leu Gly Ile Val Cys Pro Ile Cys Ser Gln | |
| 85 90 95 | |
| Lys Pro Gly Ser Met Ala Tyr Pro Tyr Asp Val Pro Asp Tyr Ala Ser | |
| 100 105 110 | |
| Leu Arg Ser Thr Met Ser Gln Ser Asn Arg Glu Leu Val Val Asp Phe | |
| 115 120 125 | |
| Leu Ser Tyr Lys Leu Ser Gln Lys Gly Tyr Ser Trp Ser Gln Phe Ser | |
| 130 135 140 | |
| Asp Val Glu Glu Asn Arg Thr Glu Ala Pro Glu Gly Thr Glu Ser Glu | |
| 145 150 155 160 | |
| Met Glu Thr Pro Ser Ala Ile Asn Gly Asn Pro Ser Trp His Leu Ala | |
| 165 170 175 | |
| Asp Ser Pro Ala Val Asn Gly Ala Thr Ala His Ser Ser Ser Leu Asp | |
| 180 185 190 | |
| Ala Arg Glu Val Ile Pro Met Ala Ala Val Lys Gln Ala Leu Arg Glu | |
| 195 200 205 | |
| Ala Gly Asp Glu Phe Glu Leu Arg Tyr Arg Arg Ala Phe Ser Asp Leu | |
| 210 215 220 | |
| Thr Ser Gln Leu His Ile Thr Pro Gly Thr Ala Tyr Gln Ser Phe Glu | |
| 225 230 235 240 | |
| Gln Val Val Asn Glu Leu Phe Arg Asp Gly Val Asn Trp Gly Arg Ile | |
| 245 250 255 | |
| Val Ala Phe Phe Ser Phe Gly Gly Ala Leu Cys Val Glu Ser Val Asp | |
| 260 265 270 | |
| Lys Glu Met Gln Val Leu Val Ser Arg Ile Ala Ala Trp Met Ala Thr | |
| 275 280 285 | |
| Tyr Leu Asn Asp His Leu Glu Pro Trp Ile Gln Glu Asn Gly Gly Trp | |
| 290 295 300 | |
| Asp Thr Phe Val Glu Leu Tyr Gly Asn Asn Ala Ala Ala Glu Ser Arg | |
| 305 310 315 320 | |
| Lys Gly Gln Glu Arg Phe Asn Arg Trp Phe Leu Thr Gly Met Thr Val | |
| 325 330 335 | |
| Ala Gly Val Val Leu Leu Gly Ser Leu Phe Ser Arg Lys | |
| 340 345 | |
| SEQ ID NO: 60 | |
| atggcgtacc catacgatgt tccagattac gctagcttga gatctaccat gtctcagagc | 60 |
| aaccgggagc tggtggttga ctttctctcc tacaagcttt cccagaaagg atacagctgg | 120 |
| agtcagttta gtgatgtgga agagaacagg actgaggccc cagaagggac tgaatcggag | 180 |
| atggagaccc ccagtgccat caatggcaac ccatcctggc acctggcaga cagccccgcg | 240 |
| gtgaatggag ccactgcgca cagcagcagt ttggatgccc gggaggtgat ccccatggca | 300 |
| gcagtaaagc aagcgctgag ggaggcaggc gacgagtttg aactgcggta ccggcgggca | 360 |
| ttcagtgacc tgacatccca gctccacatc accccaggga cagcatatca gagctttgaa | 420 |
| caggtagtga atgaactctt ccgggatggg gtagccattc ttcgcattgt ggcctttttc | 480 |
| tccttcggcg gggcactgtg cgtggaaagc gtagacaagg agatgcaggt attggtgagt | 540 |
| cggatcgcag cttggatggc cacttacctg aatgaccacc tagagccttg gatccaggag | 600 |
| aacggcggct gggatacttt tgtggaactc tatgggaaca atgcagcagc cgagagccga | 660 |
| aagggccagg aacgcttcaa ccgctggttc ctgacgggca tgactgtggc cggcgtggtt | 720 |
| ctgctgggct cactcttcag tcggaaatga | 750 |
| SEQ ID NO: 61 | |
| Met Ala Tyr Pro Tyr Asp Val Pro Asp Tyr Ala Ser Leu Arg Ser Thr | |
| 1 5 10 15 | |
| Met Ser Gln Ser Asn Arg Glu Leu Val Val Asp Phe Leu Ser Tyr Lys | |
| 20 25 30 | |
| Leu Ser Gln Lys Gly Tyr Ser Trp Ser Gln Phe Ser Asp Val Glu Glu | |
| 35 40 45 | |
| Asn Arg Thr Glu Ala Pro Glu Gly Thr Glu Ser Glu Met Glu Thr Pro | |
| 50 55 60 | |
| Ser Ala Ile Asn Gly Asn Pro Ser Trp His Leu Ala Asp Ser Pro Ala | |
| 65 70 75 80 | |
| Val Asn Gly Ala Thr Ala His Ser Ser Ser Leu Asp Ala Arg Glu Val | |
| 85 90 95 | |
| Ile Pro Met Ala Ala Val Lys Gln Ala Leu Arg Glu Ala Gly Asp Glu | |
| 100 105 110 | |
| Phe Glu Leu Arg Tyr Arg Arg Ala Phe Ser Asp Leu Thr Ser Gln Leu | |
| 115 120 125 | |
| His Ile Thr Pro Gly Thr Ala Tyr Gln Ser Phe Glu Gln Val Val Asn | |
| 130 135 140 | |
| Glu Leu Phe Arg Asp Gly Val Ala Ile Leu Arg Ile Val Ala Phe Phe | |
| 145 150 155 160 | |
| Ser Phe Gly Gly Ala Leu Cys Val Glu Ser Val Asp Lys Glu Met Gln | |
| 165 170 175 | |
| Val Leu Val Ser Arg Ile Ala Ala Trp Met Ala Thr Tyr Leu Asn Asp | |
| 180 185 190 | |
| His Leu Glu Pro Trp Ile Gln Glu Asn Gly Gly Trp Asp Thr Phe Val | |
| 195 200 205 | |
| Glu Leu Tyr Gly Asn Asn Ala Ala Ala Glu Ser Arg Lys Gly Gln Glu | |
| 210 215 220 | |
| Arg Phe Asn Arg Trp Phe Leu Thr Gly Met Thr Val Ala Gly Val Val | |
| 225 230 235 240 | |
| Leu Leu Gly Ser Leu Phe Ser Arg Lys | |
| 245 | |
| SEQ ID NO: 62 | |
| Met His Gly Asp Thr Pro Thr Leu His Glu Tyr Met Leu Asp Leu Gln | |
| 1 5 10 15 | |
| Pro Glu Thr Thr Asp Leu Tyr Cys Tyr Glu Gln Leu Asn Asp Ser Ser | |
| 20 25 30 | |
| Glu Glu Glu Asp Glu Ile Asp Gly Pro Ala Gly Gln Ala Glu Pro Asp | |
| 35 40 45 | |
| Arg Ala His Tyr Asn Ile Val Thr Phe Cys Cys Lys Cys Asp Ser Thr | |
| 50 55 60 | |
| Leu Arg Leu Cys Val Gln Ser Thr His Val Asp Ile Arg Thr Leu Glu | |
| 65 70 75 80 | |
| Asp Leu Leu Met Gly Thr Leu Gly Ile Val Cys Pro Ile Cys Ser Gln | |
| 85 90 95 | |
| Lys Pro Gly Ser Met Ala Tyr Pro Tyr Asp Val Pro Asp Tyr Ala Ser | |
| 100 105 110 | |
| Leu Arg Ser Thr Met Ser Gln Ser Asn Arg Glu Leu Val Val Asp Phe | |
| 115 120 125 | |
| Leu Ser Tyr Lys Leu Ser Gln Lys Gly Tyr Ser Trp Ser Gln Phe Ser | |
| 130 135 140 | |
| Asp Val Glu Glu Asn Arg Thr Glu Ala Pro Glu Gly Thr Glu Ser Glu | |
| 145 150 155 160 | |
| Met Glu Thr Pro Ser Ala Ile Asn Gly Asn Pro Ser Trp His Leu Ala | |
| 165 170 175 | |
| Asp Ser Pro Ala Val Asn Gly Ala Thr Ala His Ser Ser Ser Leu Asp | |
| 180 185 190 | |
| Ala Arg Glu Val Ile Pro Met Ala Ala Val Lys Gln Ala Leu Arg Glu | |
| 195 200 205 | |
| Ala Gly Asp Glu Phe Glu Leu Arg Tyr Arg Arg Ala Phe Ser Asp Leu | |
| 210 215 220 | |
| Thr Ser Gln Leu His Ile Thr Pro Gly Thr Ala Tyr Gln Ser Phe Glu | |
| 225 230 235 240 | |
| Gln Val Val Asn Glu Leu Phe Arg Asp Gly Val Ala Ile Leu Arg Ile | |
| 245 250 255 | |
| Val Ala Phe Phe Ser Phe Gly Gly Ala Leu Cys Val Glu Ser Val Asp | |
| 260 265 270 | |
| Lys Glu Met Gln Val Leu Val Ser Arg Ile Ala Ala Trp Met Ala Thr | |
| 275 280 285 | |
| Tyr Leu Asn Asp His Leu Glu Pro Trp Ile Gln Glu Asn Gly Gly Trp | |
| 290 295 300 | |
| Asp Thr Phe Val Glu Leu Tyr Gly Asn Asn Ala Ala Ala Glu Ser Arg | |
| 305 310 315 320 | |
| Lys Gly Gln Glu Arg Phe Asn Arg Trp Phe Leu Thr Gly Met Thr Val | |
| 325 330 335 | |
| Ala Gly Val Val Leu Leu Gly Ser Leu Phe Ser Arg Lys | |
| 340 345 | |
| SEQ ID NO: 63 | |
| gacggatcgg gagatctccc gatcccctat ggtcgactct cagtacaatc tgctctgatg | 60 |
| ccgcatagtt aagccagtat ctgctccctg cttgtgtgtt ggaggtcgct gagtagtgcg | 120 |
| cgagcaaaat ttaagctaca acaaggcaag gcttgaccga caattgcatg aagaatctgc | 180 |
| ttagggttag gcgttttgcg ctgcttcgcg atgtacgggc cagatatacg cgttgacatt | 240 |
| gattattgac tagttattaa tagtaatcaa ttacggggtc attagttcat agcccatata | 300 |
| tggagttccg cgttacataa cttacggtaa atggcccgcc tggctgaccg cccaacgacc | 360 |
| cccgcccatt gacgtcaata atgacgtatg ttcccatagt aacgccaata gggactttcc | 420 |
| attgacgtca atgggtggac tatttacggt aaactgccca cttggcagta catcaagtgt | 480 |
| atcatatgcc aagtacgccc cctattgacg tcaatgacgg taaatggccc gcctggcatt | 540 |
| atgcccagta catgacctta tgggactttc ctacttggca gtacatctac gtattagtca | 600 |
| tcgctattac catggtgatg cggttttggc agtacatcaa tgggcgtgga tagcggtttg | 660 |
| actcacgggg atttccaagt ctccacccca ttgacgtcaa tgggagtttg ttttggcacc | 720 |
| aaaatcaacg ggactttcca aaatgtcgta acaactccgc cccattgacg caaatgggcg | 780 |
| gtaggcgtgt acggtgggag gtctatataa gcagagctct ctggctaact agagaaccca | 840 |
| ctgcttactg gcttatcgaa attaatacga ctcactatag ggagacccaa gctggctagc | 900 |
| gtttaaacgg gccctctaga ctcgagcggc cgccactgtg ctggatatct gcagaattcc | 960 |
| accacactgg actagtggat ctatggcgta cccatacgat gttccagatt acgctagctt | 1020 |
| gagatctacc atgtctcaga gcaaccggga gctggtggtt gactttctct cctacaagct | 1080 |
| ttcccagaaa ggatacagct ggagtcagtt tagtgatgtg gaagagaaca ggactgaggc | 1140 |
| cccagaaggg actgaatcgg agatggagac ccccagtgcc atcaatggca acccatcctg | 1200 |
| gcacctggca gacagccccg cggtgaatgg agccactgcg cacagcagca gtttggatgc | 1260 |
| ccgggaggtg atccccatgg cagcagtaaa gcaagcgctg agggaggcag gcgacgagtt | 1320 |
| tgaactgcgg taccggcggg cattcagtga cctgacatcc cagctccaca tcaccccagg | 1380 |
| gacagcatat cagagctttg aacaggtagt gaatgaactc ttccgggatg gggtagccat | 1440 |
| tcttcgcatt gtggcctttt tctccttcgg cggggcactg tgcgtggaaa gcgtagacaa | 1500 |
| ggagatgcag gtattggtga gtcggatcgc agcttggatg gccacttacc tgaatgacca | 1560 |
| cctagagcct tggatccagg agaacggcgg ctgggatact tttgtggaac tctatgggaa | 1620 |
| caatgcagca gccgagagcc gaaagggcca ggaacgcttc aaccgctggt tcctgacggg | 1680 |
| catgactgtg gccggcgtgg ttctgctggg ctcactcttc agtcggaaat gaagatccga | 1740 |
| gctcggtacc aagcttaagt ttaaaccgct gatcagcctc gactgtgcct tctagttgcc | 1800 |
| agccatctgt tgtttgcccc tcccccgtgc cttccttgac cctggaaggt gccactccca | 1860 |
| ctgtcctttc ctaataaaat gaggaaaatg catcgcattg tctgagtagg tgtcattcta | 1920 |
| ttctgggggg tggggtgggg caggacagca agggggagga ttgggaagac aatagcaggc | 1980 |
| atgctgggga tgcggtgggc tctatggctt ctgaggcgga aagaaccagc tggggctcta | 2040 |
| gggggtatcc ccacgcgccc tgtagcggcg cattaagcgc ggcgggtgtg gtggttacgc | 2100 |
| gcagcgtgac cgctacactt gccagcgccc tagcgcccgc tcctttcgct ttcttccctt | 2160 |
| cctttctcgc cacgttcgcc ggctttcccc gtcaagctct aaatcggggc atccctttag | 2220 |
| ggttccgatt tagtgcttta cggcacctcg accccaaaaa acttgattag ggtgatggtt | 2280 |
| cacgtagtgg gccatcgccc tgatagacgg tttttcgccc tttgacgttg gagtccacgt | 2340 |
| tctttaatag tggactcttg ttccaaactg gaacaacact caaccctatc tcggtctatt | 2400 |
| cttttgattt ataagggatt ttggggattt cggcctattg gttaaaaaat gagctgattt | 2460 |
| aacaaaaatt taacgcgaat taattctgtg gaatgtgtgt cagttagggt gtggaaagtc | 2520 |
| cccaggctcc ccaggcaggc agaagtatgc aaagcatgca tctcaattag tcagcaacca | 2580 |
| ggtgtggaaa gtccccaggc tccccagcag gcagaagtat gcaaagcatg catctcaatt | 2640 |
| agtcagcaac catagtcccg cccctaactc cgcccatccc gcccctaact ccgcccagtt | 2700 |
| ccgcccattc tccgccccat ggctgactaa ttttttttat ttatgcagag gccgaggccg | 2760 |
| cctctgcctc tgagctattc cagaagtagt gaggaggctt ttttggaggc ctaggctttt | 2820 |
| gcaaaaagct cccgggagct tgtatatcca ttttcggatc tgatcaagag acaggatgag | 2880 |
| gatcgtttcg catgattgaa caagatggat tgcacgcagg ttctccggcc gcttgggtgg | 2940 |
| agaggctatt cggctatgac tgggcacaac agacaatcgg ctgctctgat gccgccgtgt | 3000 |
| tccggctgtc agcgcagggg cgcccggttc tttttgtcaa gaccgacctg tccggtgccc | 3060 |
| tgaatgaact gcaggacgag gcagcgcggc tatcgtggct ggccacgacg ggcgttcctt | 3120 |
| gcgcagctgt gctcgacgtt gtcactgaag cgggaaggga ctggctgcta ttgggcgaag | 3180 |
| tgccggggca ggatctcctg tcatctcacc ttgctcctgc cgagaaagta tccatcatgg | 3240 |
| ctgatgcaat gcggcggctg catacgcttg atccggctac ctgcccattc gaccaccaag | 3300 |
| cgaaacatcg catcgagcga gcacgtactc ggatggaagc cggtcttgtc gatcaggatg | 3360 |
| atctggacga agagcatcag gggctcgcgc cagccgaact gttcgccagg ctcaaggcgc | 3420 |
| gcatgcccga cggcgaggat ctcgtcgtga cccatggcga tgcctgcttg ccgaatatca | 3480 |
| tggtggaaaa tggccgcttt tctggattca tcgactgtgg ccggctgggt gtggcggacc | 3540 |
| gctatcagga catagcgttg gctacccgtg atattgctga agagcttggc ggcgaatggg | 3600 |
| ctgaccgctt cctcgtgctt tacggtatcg ccgctcccga ttcgcagcgc atcgccttct | 3660 |
| atcgccttct tgacgagttc ttctgagcgg gactctgggg ttcgaaatga ccgaccaagc | 3720 |
| gacgcccaac ctgccatcac gagatttcga ttccaccgcc gccttctatg aaaggttggg | 3780 |
| cttcggaatc gttttccggg acgccggctg gatgatcctc cagcgcgggg atctcatgct | 3840 |
| ggagttcttc gcccacccca acttgtttat tgcagcttat aatggttaca aataaagcaa | 3900 |
| tagcatcaca aatttcacaa ataaagcatt tttttcactg cattctagtt gtggtttgtc | 3960 |
| caaactcatc aatgtatctt atcatgtctg tataccgtcg acctctagct agagcttggc | 4020 |
| gtaatcatgg tcatagctgt ttcctgtgtg aaattgttat ccgctcacaa ttccacacaa | 4080 |
| catacgagcc ggaagcataa agtgtaaagc ctggggtgcc taatgagtga gctaactcac | 4140 |
| attaattgcg ttgcgctcac tgcccgcttt ccagtcggga aacctgtcgt gccagctgca | 4200 |
| ttaatgaatc ggccaacgcg cggggagagg cggtttgcgt attgggcgct cttccgcttc | 4260 |
| ctcgctcact gactcgctgc gctcggtcgt tcggctgcgg cgagcggtat cagctcactc | 4320 |
| aaaggcggta atacggttat ccacagaatc aggggataac gcaggaaaga acatgtgagc | 4380 |
| aaaaggccag caaaaggcca ggaaccgtaa aaaggccgcg ttgctggcgt ttttccatag | 4440 |
| gctccgcccc cctgacgagc atcacaaaaa tcgacgctca agtcagaggt ggcgaaaccc | 4500 |
| gacaggacta taaagatacc aggcgtttcc ccctggaagc tccctcgtgc gctctcctgt | 4560 |
| tccgaccctg ccgcttaccg gatacctgtc cgcctttctc ccttcgggaa gcgtggcgct | 4620 |
| ttctcaatgc tcacgctgta ggtatctcag ttcggtgtag gtcgttcgct ccaagctggg | 4680 |
| ctgtgtgcac gaaccccccg ttcagcccga ccgctgcgcc ttatccggta actatcgtct | 4740 |
| tgagtccaac ccggtaagac acgacttatc gccactggca gcagccactg gtaacaggat | 4800 |
| tagcagagcg aggtatgtag gcggtgctac agagttcttg aagtggtggc ctaactacgg | 4860 |
| ctacactaga aggacagtat ttggtatctg cgctctgctg aagccagtta ccttcggaaa | 4920 |
| aagagttggt agctcttgat ccggcaaaca aaccaccgct ggtagcggtg gtttttttgt | 4980 |
| ttgcaagcag cagattacgc gcagaaaaaa aggatctcaa gaagatcctt tgatcttttc | 5040 |
| tacggggtct gacgctcagt ggaacgaaaa ctcacgttaa gggattttgg tcatgagatt | 5100 |
| atcaaaaagg atcttcacct agatcctttt aaattaaaaa tgaagtttta aatcaatcta | 5160 |
| aagtatatat gagtaaactt ggtctgacag ttaccaatgc ttaatcagtg aggcacctat | 5220 |
| ctcagcgatc tgtctatttc gttcatccat agttgcctga ctccccgtcg tgtagataac | 5280 |
| tacgatacgg gagggcttac catctggccc cagtgctgca atgataccgc gagacccacg | 5340 |
| ctcaccggct ccagatttat cagcaataaa ccagccagcc ggaagggccg agcgcagaag | 5400 |
| tggtcctgca actttatccg cctccatcca gtctattaat tgttgccggg aagctagagt | 5460 |
| aagtagttcg ccagttaata gtttgcgcaa cgttgttgcc attgctacag gcatcgtggt | 5520 |
| gtcacgctcg tcgtttggta tggcttcatt cagctccggt tcccaacgat caaggcgagt | 5580 |
| tacatgatcc cccatgttgt gcaaaaaagc ggttagctcc ttcggtcctc cgatcgttgt | 5640 |
| cagaagtaag ttggccgcag tgttatcact catggttatg gcagcactgc ataattctct | 5700 |
| tactgtcatg ccatccgtaa gatgcttttc tgtgactggt gagtactcaa ccaagtcatt | 5760 |
| ctgagaatag tgtatgcggc gaccgagttg ctcttgcccg gcgtcaatac gggataatac | 5820 |
| cgcgccacat agcagaactt taaaagtgct catcattgga aaacgttctt cggggcgaaa | 5880 |
| actctcaagg atcttaccgc tgttgagatc cagttcgatg taacccactc gtgcacccaa | 5940 |
| ctgatcttca gcatctttta ctttcaccag cgtttctggg tgagcaaaaa caggaaggca | 6000 |
| aaatgccgca aaaaagggaa taagggcgac acggaaatgt tgaatactca tactcttcct | 6060 |
| ttttcaatat tattgaagca tttatcaggg ttattgtctc atgagcggat acatatttga | 6120 |
| atgtatttag aaaaataaac aaataggggt tccgcgcaca tttccccgaa aagtgccacc | 6180 |
| tgacgtc | 6187 |
| SEQ ID NO: 64 | |
| acggatcgg gagatctccc gatcccctat ggtcgactct cagtacaatc tgctctgatg | 60 |
| ccgcatagtt aagccagtat ctgctccctg cttgtgtgtt ggaggtcgct gagtagtgcg | 120 |
| cgagcaaaat ttaagctaca acaaggcaag gcttgaccga caattgcatg aagaatctgc | 180 |
| ttagggttag gcgttttgcg ctgcttcgcg atgtacgggc cagatatacg cgttgacatt | 240 |
| gattattgac tagttattaa tagtaatcaa ttacggggtc attagttcat agcccatata | 300 |
| tggagttccg cgttacataa cttacggtaa atggcccgcc tggctgaccg cccaacgacc | 360 |
| cccgcccatt gacgtcaata atgacgtatg ttcccatagt aacgccaata gggactttcc | 420 |
| attgacgtca atgggtggac tatttacggt aaactgccca cttggcagta catcaagtgt | 480 |
| atcatatgcc aagtacgccc cctattgacg tcaatgacgg taaatggccc gcctggcatt | 540 |
| atgcccagta catgacctta tgggactttc ctacttggca gtacatctac gtattagtca | 600 |
| tcgctattac catggtgatg cggttttggc agtacatcaa tgggcgtgga tagcggtttg | 660 |
| actcacgggg atttccaagt ctccacccca ttgacgtcaa tgggagtttg ttttggcacc | 720 |
| aaaatcaacg ggactttcca aaatgtcgta acaactccgc cccattgacg caaatgggcg | 780 |
| gtaggcgtgt acggtgggag gtctatataa gcagagctct ctggctaact agagaaccca | 840 |
| ctgcttactg gcttatcgaa attaatacga ctcactatag ggagacccaa gctggctagc | 900 |
| gtttaaacgg gccctctaga ctcgagcggc cgccactgtg ctggatatct gcagaattca | 960 |
| tgcatggaga tacacctaca ttgcatgaat atatgttaga tttgcaacca gagacaactg | 1020 |
| atctctactg ttatgagcaa ttaaatgaca gctcagagga ggaggatgaa atagatggtc | 1080 |
| cagctggaca agcagaaccg gacagagccc attacaatat tgtaaccttt tgttgcaagt | 1140 |
| gtgactctac gcttcggttg tgcgtacaaa gcacacacgt agacattcgt actttggaag | 1200 |
| acctgttaat gggcacacta ggaattgtgt gccccatctg ttctcagaaa ccaggatcta | 1260 |
| tggcgtaccc atacgatgtt ccagattacg ctagcttgag atctaccatg tctcagagca | 1320 |
| accgggagct ggtggttgac tttctctcct acaagctttc ccagaaagga tacagctgga | 1380 |
| gtcagtttag tgatgtggaa gagaacagga ctgaggcccc agaagggact gaatcggaga | 1440 |
| tggagacccc cagtgccatc aatggcaacc catcctggca cctggcagac agccccgcgg | 1500 |
| tgaatggagc cactgcgcac agcagcagtt tggatgcccg ggaggtgatc cccatggcag | 1560 |
| cagtaaagca agcgctgagg gaggcaggcg acgagtttga actgcggtac cggcgggcat | 1620 |
| tcagtgacct gacatcccag ctccacatca ccccagggac agcatatcag agctttgaac | 1680 |
| aggtagtgaa tgaactcttc cgggatgggg tagccattct tcgcattgtg gcctttttct | 1740 |
| ccttcggcgg ggcactgtgc gtggaaagcg tagacaagga gatgcaggta ttggtgagtc | 1800 |
| ggatcgcagc ttggatggcc acttacctga atgaccacct agagccttgg atccaggaga | 1860 |
| acggcggctg ggatactttt gtggaactct atgggaacaa tgcagcagcc gagagccgaa | 1920 |
| agggccagga acgcttcaac cgctggttcc tgacgggcat gactgtggcc ggcgtggttc | 1980 |
| tactgggctc actcttcagt cggaaatgaa gatccaagct taagtttaaa ccgctgatca | 2040 |
| gcctcgactg tgccttctag ttgccagcca tctgttgttt gcccctcccc cgtgccttcc | 2100 |
| ttgaccctgg aaggtgccac tcccactgtc ctttcctaat aaaatgagga aattgcatcg | 2160 |
| cattgtctga gtaggtgtca ttctattctg gggggtgggg tggggcagga cagcaagggg | 2220 |
| gaggattggg aagacaatag caggcatgct ggggatgcgg tgggctctat ggcttctgag | 2280 |
| gcggaaagaa ccagctgggg ctctaggggg tatccccacg cgccctgtag cggcgcatta | 2340 |
| agcgcggcgg gtgtggtggt tacgcgcagc gtgaccgcta cacttgccag cgccctagcg | 2400 |
| cccgctcctt tcgctttctt cccttccttt ctcgccacgt tcgccggctt tccccgtcaa | 2460 |
| gctctaaatc ggggcatccc tttagggttc cgatttagtg ctttacggca cctcgacccc | 2520 |
| aaaaaacttg attagggtga tggttcacgt agtgggccat cgccctgata gacggttttt | 2580 |
| cgccctttga cgttggagtc cacgttcttt aatagtggac tcttgttcca aactggaaca | 2640 |
| acactcaacc ctatctcggt ctattctttt gatttataag ggattttggg gatttcggcc | 2700 |
| tattggttaa aaaatgagct gatttaacaa aaatttaacg cgaattaatt ctgtggaatg | 2760 |
| tgtgtcagtt agggtgtgga aagtccccag gctccccagg caggcagaag tatgcaaagc | 2820 |
| atgcatctca attagtcagc aaccaggtgt ggaaagtccc caggctcccc agcaggcaga | 2880 |
| agtatgcaaa gcatgcatct caattagtca gcaaccatag tcccgcccct aactccgccc | 2940 |
| atcccgcccc taactccgcc cagttccgcc cattctccgc cccatggctg actaattttt | 3000 |
| tttatttatg cagaggccga ggccgcctct gcctctgagc tattccagaa gtagtgagga | 3060 |
| ggcttttttg gaggcctagg cttttgcaaa aagctcccgg gagcttgtat atccattttc | 3120 |
| ggatctgatc aagagacagg atgaggatcg tttcgcatga ttgaacaaga tggattgcac | 3180 |
| gcaggttctc cggccgcttg ggtggagagg ctattcggct atgactgggc acaacagaca | 3240 |
| atcggctgct ctgatgccgc cgtgttccgg ctgtcagcgc aggggcgccc ggttcttttt | 3300 |
| gtcaagaccg acctgtccgg tgccctgaat gaactgcagg acgaggcagc gcggctatcg | 3360 |
| tggctggcca cgacgggcgt tccttgcgca gctgtgctcg acgttgtcac tgaagcggga | 3420 |
| agggactggc tgctattggg cgaagtgccg gggcaggatc tcctgtcatc tcaccttgct | 3480 |
| cctgccgaga aagtatccat catggctgat gcaatgcggc ggctgcatac gcttgatccg | 3540 |
| gctacctgcc cattcgacca ccaagcgaaa catcgcatcg agcgagcacg tactcggatg | 3600 |
| gaagccggtc ttgtcgatca ggatgatctg gacgaagagc atcaggggct cgcgccagcc | 3660 |
| gaactgttcg ccaggctcaa ggcgcgcatg cccgacggcg aggatctcgt cgtgacccat | 3720 |
| ggcgatgcct gcttgccgaa tatcatggtg gaaaatggcc gcttttctgg attcatcgac | 3780 |
| tgtggccggc tgggtgtggc ggaccgctat caggacatag cgttggctac ccgtgatatt | 3840 |
| gctgaagagc ttggcggcga atgggctgac cgcttcctcg tgctttacgg tatcgccgct | 3900 |
| cccgattcgc agcgcatcgc cttctatcgc cttcttgacg agttcttctg agcgggactc | 3960 |
| tggggttcga aatgaccgac caagcgacgc ccaacctgcc atcacgagat ttcgattcca | 4020 |
| ccgccgcctt ctatgaaagg ttgggcttcg gaatcgtttt ccgggacgcc ggctggatga | 4080 |
| tcctccagcg cggggatctc atgctggagt tcttcgccca ccccaacttg tttattgcag | 4140 |
| cttataatgg ttacaaataa agcaatagca tcacaaattt cacaaataaa gcattttttt | 4200 |
| cactgcattc tagttgtggt ttgtccaaac tcatcaatgt atcttatcat gtctgtatac | 4260 |
| cgtcgacctc tagctagagc ttggcgtaat catggtcata gctgtttcct gtgtgaaatt | 4320 |
| gttatccgct cacaattcca cacaacatac gagccggaag cataaagtgt aaagcctggg | 4380 |
| gtgcctaatg agtgagctaa ctcacattaa ttgcgttgcg ctcactgccc gctttccagt | 4440 |
| cgggaaacct gtcgtgccag ctgcattaat gaatcggcca acgcgcgggg agaggcggtt | 4500 |
| tgcgtattgg gcgctcttcc gcttcctcgc tcactgactc gctgcgctcg gtcgttcggc | 4560 |
| tgcggcgagc ggtatcagct cactcaaagg cggtaatacg gttatccaca gaatcagggg | 4620 |
| ataacgcagg aaagaacatg tgagcaaaag gccagcaaaa ggccaggaac cgtaaaaagg | 4680 |
| ccgcgttgct ggcgtttttc cataggctcc gcccccctga cgagcatcac aaaaatcgac | 4740 |
| gctcaagtca gaggtggcga aacccgacag gactataaag ataccaggcg tttccccctg | 4800 |
| gaagctccct cgtgcgctct cctgttccga ccctgccgct taccggatac ctgtccgcct | 4860 |
| ttctcccttc gggaagcgtg gcgctttctc aatgctcacg ctgtaggtat ctcagttcgg | 4920 |
| tgtaggtcgt tcgctccaag ctgggctgtg tgcacgaacc ccccgttcag cccgaccgct | 4980 |
| gcgccttatc cggtaactat cgtcttgagt ccaacccggt aagacacgac ttatcgccac | 5040 |
| tggcagcagc cactggtaac aggattagca gagcgaggta tgtaggcggt gctacagagt | 5100 |
| tcttgaagtg gtggcctaac tacggctaca ctagaaggac agtatttggt atctgcgctc | 5160 |
| tgctgaagcc agttaccttc ggaaaaagag ttggtagctc ttgatccggc aaacaaacca | 5220 |
| ccgctggtag cggtggtttt tttgtttgca agcagcagat tacgcgcaga aaaaaaggat | 5280 |
| ctcaagaaga tcctttgatc ttttctacgg ggtctgacgc tcagtggaac gaaaactcac | 5340 |
| gttaagggat tttggtcatg agattatcaa aaaggatctt cacctagatc cttttaaatt | 5400 |
| aaaaatgaag ttttaaatca atctaaagta tatatgagta aacttggtct gacagttacc | 5460 |
| aatgcttaat cagtgaggca cctatctcag cgatctgtct atttcgttca tccatagttg | 5520 |
| cctgactccc cgtcgtgtag ataactacga tacgggaggg cttaccatct ggccccagtg | 5580 |
| ctgcaatgat accgcgagac ccacgctcac cggctccaga tttatcagca ataaaccagc | 5640 |
| cagccggaag ggccgagcgc agaagtggtc ctgcaacttt atccgcctcc atccagtcta | 5700 |
| ttaattgttg ccgggaagct agagtaagta gttcgccagt taatagtttg cgcaacgttg | 5760 |
| ttgccattgc tacaggcatc gtggtgtcac gctcgtcgtt tggtatggct tcattcagct | 5820 |
| ccggttccca acgatcaagg cgagttacat gatcccccat gttgtgcaaa aaagcggtta | 5880 |
| gctccttcgg tcctccgatc gttgtcagaa gtaagttggc cgcagtgtta tcactcatgg | 5940 |
| ttatggcagc actgcataat tctcttactg tcatgccatc cgtaagatgc ttttctgtga | 6000 |
| ctggtgagta ctcaaccaag tcattctgag aatagtgtat gcggcgaccg agttgctctt | 6060 |
| gcccggcgtc aatacgggat aataccgcgc cacatagcag aactttaaaa gtgctcatca | 6120 |
| ttggaaaacg ttcttcgggg cgaaaactct caaggatctt accgctgttg agatccagtt | 6180 |
| cgatgtaacc cactcgtgca cccaactgat cttcagcatc ttttactttc accagcgttt | 6240 |
| ctgggtgagc aaaaacagga aggcaaaatg ccgcaaaaaa gggaataagg gcgacacgga | 6300 |
| aatgttgaat actcatactc ttcctttttc aatattattg aagcatttat cagggttatt | 6360 |
| gtctcatgag cggatacata tttgaatgta tttagaaaaa taaacaaata ggggttccgc | 6420 |
| gcacatttcc ccgaaaagtg ccacctgacg tc | 6452 |
| SEQ ID NO: 65 | |
| atggcggatg tgtgacatac acgacgccaa aagattttgt tccagctcct gccacctccg | 60 |
| ctacgcgaga gattaaccac ccacgatggc cgccaaagtg catgttgata ttgaggctga | 120 |
| cagcccattc atcaagtctt tgcagaaggc atttccgtcg ttcgaggtgg agtcattgca | 180 |
| ggtcacacca aatgaccatg caaatgccag agcattttcg cacctggcta ccaaattgat | 240 |
| cgagcaggag actgacaaag acacactcat cttggatatc ggcagtgcgc cttccaggag | 300 |
| aatgatgtct acgcacaaat accactgcgt atgccctatg cgcagcgcag aagaccccga | 360 |
| aaggctcgat agctacgcaa agaaactggc agcggcctcc gggaaggtgc tggatagaga | 420 |
| gatcgcagga aaaatcaccg acctgcagac cgtcatggct acgccagacg ctgaatctcc | 480 |
| taccttttgc ctgcatacag acgtcacgtg tcgtacggca gccgaagtgg ccgtatacca | 540 |
| ggacgtgtat gctgtacatg caccaacatc gctgtaccat caggcgatga aaggtgtcag | 600 |
| aacggcgtat tggattgggt ttgacaccac cccgtttatg tttgacgcgc tagcaggcgc | 660 |
| gtatccaacc tacgccacaa actgggccga cgagcaggtg ttacaggcca ggaacatagg | 720 |
| actgtgtgca gcatccttga ctgagggaag actcggcaaa ctgtccattc tccgcaagaa | 780 |
| gcaattgaaa ccttgcgaca cagtcatgtt ctcggtagga tctacattgt acactgagag | 840 |
| cagaaagcta ctgaggagct ggcacttacc ctccgtattc cacctgaaag gtaaacaatc | 900 |
| ctttacctgt aggtgcgata ccatcgtatc atgtgaaggg tacgtagtta agaaaatcac | 960 |
| tatgtgcccc ggcctgtacg gtaaaacggt agggtacgcc gtgacgtatc acgcggaggg | 1020 |
| attcctagtg tgcaagacca cagacactgt caaaggagaa agagtctcat tccctgtatg | 1080 |
| cacctacgtc ccctcaacca tctgtgatca aatgactggc atactagcga ccgacgtcac | 1140 |
| accggaggac gcacagaagt tgttagtggg attgaatcag aggatagttg tgaacggaag | 1200 |
| aacacagcga aacactaaca cgatgaagaa ctatctgctt ccgattgtgg ccgtcgcatt | 1260 |
| tagcaagtgg gcgagggaat acaaggcaga ccttgatgat gaaaaacctc tgggtgtccg | 1320 |
| agagaggtca cttacttgct gctgcttgtg ggcatttaaa acgaggaaga tgcacaccat | 1380 |
| gtacaagaaa ccagacaccc agacaatagt gaaggtgcct tcagagttta actcgttcgt | 1440 |
| catcccgagc ctatggtcta caggcctcgc aatcccagtc agatcacgca ttaagatgct | 1500 |
| tttggccaag aagaccaagc gagagttaat acctgttctc gacgcgtcgt cagccaggga | 1560 |
| tgctgaacaa gaggagaagg agaggttgga ggccgagctg actagagaag ccttaccacc | 1620 |
| cctcgtcccc atcgcgccgg cggagacggg agtcgtcgac gtcgacgttg aagaactaga | 1680 |
| gtatcacgca ggtgcagggg tcgtggaaac acctcgcagc gcgttgaaag tcaccgcaca | 1740 |
| gccgaacgac gtactactag gaaattacgt agttctgtcc ccgcagaccg tgctcaagag | 1800 |
| ctccaagttg gcccccgtgc accctctagc agagcaggtg aaaataataa cacataacgg | 1860 |
| gagggccggc ggttaccagg tcgacggata tgacggcagg gtcctactac catgtggatc | 1920 |
| ggccattccg gtccctgagt ttcaagcttt gagcgagagc gccactatgg tgtacaacga | 1980 |
| aagggagttc gtcaacagga aactatacca tattgccgtt cacggaccgt cgctgaacac | 2040 |
| cgacgaggag aactacgaga aagtcagagc tgaaagaact gacgccgagt acgtgttcga | 2100 |
| cgtagataaa aaatgctgcg tcaagagaga ggaagcgtcg ggtttggtgt tggtgggaga | 2160 |
| gctaaccaac cccccgttcc atgaattcgc ctacgaaggg ctgaagatca ggccgtcggc | 2220 |
| accatataag actacagtag taggagtctt tggggttccg ggatcaggca agtctgctat | 2280 |
| tattaagagc ctcgtgacca aacacgatct ggtcaccagc ggcaagaagg agaactgcca | 2340 |
| ggaaatagtt aacgacgtga agaagcaccg cgggaagggg acaagtaggg aaaacagtga | 2400 |
| ctccatcctg ctaaacgggt gtcgtcgtgc cgtggacatc ctatatgtgg acgaggcttt | 2460 |
| cgctagccat tccggtactc tgctggccct aattgctctt gttaaacctc ggagcaaagt | 2520 |
| ggtgttatgc ggagacccca agcaatgcgg attcttcaat atgatgcagc ttaaggtgaa | 2580 |
| cttcaaccac aacatctgca ctgaagtatg tcataaaagt atatccagac gttgcacgcg | 2640 |
| tccagtcacg gccatcgtgt ctacgttgca ctacggaggc aagatgcgca cgaccaaccc | 2700 |
| gtgcaacaaa cccataatca tagacaccac aggacagacc aagcccaagc caggagacat | 2760 |
| cgtgttaaca tgcttccgag gctgggcaaa gcagctgcag ttggactacc gtggacacga | 2820 |
| agtcatgaca gcagcagcat ctcagggcct cacccgcaaa ggggtatacg ccgtaaggca | 2880 |
| gaaggtgaat gaaaatccct tgtatgcccc tgcgtcggag cacgtgaatg tactgctgac | 2940 |
| gcgcactgag gataggctgg tgtggaaaac gctggccggc gatccctgga ttaaggtcct | 3000 |
| atcaaacatt ccacagggta actttacggc cacattggaa gaatggcaag aagaacacga | 3060 |
| caaaataatg aaggtgattg aaggaccggc tgcgcctgtg gacgcgttcc agaacaaagc | 3120 |
| gaacgtgtgt tgggcgaaaa gcctggtgcc tgtcctggac actgccggaa tcagattgac | 3180 |
| agcagaggag tggagcacca taattacagc atttaaggag gacagagctt actctccagt | 3240 |
| ggtggccttg aatgaaattt gcaccaagta ctatggagtt gacctggaca gtggcctgtt | 3300 |
| ttctgccccg aaggtgtccc tgtattacga gaacaaccac tgggataaca gacctggtgg | 3360 |
| aaggatgtat ggattcaatg ccgcaacagc tgccaggctg gaagctagac ataccttcct | 3420 |
| gaaggggcag tggcatacgg gcaagcaggc agttatcgca gaaagaaaaa tccaaccgct | 3480 |
| ttctgtgctg gacaatgtaa ttcctatcaa ccgcaggctg ccgcacgccc tggtggctga | 3540 |
| gtacaagacg gttaaaggca gtagggttga gtggctggtc aataaagtaa gagggtacca | 3600 |
| cgtcctgctg gtgagtgagt acaacctggc tttgcctcga cgcagggtca cttggttgtc | 3660 |
| accgctgaat gtcacaggcg ccgataggtg ctacgaccta agtttaggac tgccggctga | 3720 |
| cgccggcagg ttcgacttgg tctttgtgaa cattcacacg gaattcagaa tccaccacta | 3780 |
| ccagcagtgt gtcgaccacg ccatgaagct gcagatgctt gggggagatg cgctacgact | 3840 |
| gctaaaaccc ggcggcatct tgatgagagc ttacggatac gccgataaaa tcagcgaagc | 3900 |
| cgttgtttcc tccttaagca gaaagttctc gtctgcaaga gtgttgcgcc cggattgtgt | 3960 |
| caccagcaat acagaagtgt tcttgctgtt ctccaacttt gacaacggaa agagaccctc | 4020 |
| tacgctacac cagatgaata ccaagctgag tgccgtgtat gccggagaag ccatgcacac | 4080 |
| ggccgggtgt gcaccatcct acagagttaa gagagcagac atagccacgt gcacagaagc | 4140 |
| ggctgtggtt aacgcagcta acgcccgtgg aactgtaggg gatggcgtat gcagggccgt | 4200 |
| ggcgaagaaa tggccgtcag cctttaaggg agcagcaaca ccagtgggca caattaaaac | 4260 |
| agtcatgtgc ggctcgtacc ccgtcatcca cgctgtagcg cctaatttct ctgccacgac | 4320 |
| tgaagcggaa ggggaccgcg aattggccgc tgtctaccgg gcagtggccg ccgaagtaaa | 4380 |
| cagactgtca ctgagcagcg tagccatccc gctgctgtcc acaggagtgt tcagcggcgg | 4440 |
| aagagatagg ctgcagcaat ccctcaacca tctattcaca gcaatggacg ccacggacgc | 4500 |
| tgacgtgacc atctactgca gagacaaaag ttgggagaag aaaatccagg aagccattga | 4560 |
| catgaggacg gctgtggagt tgctcaatga tgacgtggag ctgaccacag acttggtgag | 4620 |
| agtgcacccg gacagcagcc tggtgggtcg taagggctac agtaccactg acgggtcgct | 4680 |
| gtactcgtac tttgaaggta cgaaattcaa ccaggctgct attgatatgg cagagatact | 4740 |
| gacgttgtgg cccagactgc aagaggcaaa cgaacagata tgcctatacg cgctgggcga | 4800 |
| aacaatggac aacatcagat ccaaatgtcc ggtgaacgat tccgattcat caacacctcc | 4860 |
| caggacagtg ccctgcctgt gccgctacgc aatgacagca gaacggatcg cccgccttag | 4920 |
| gtcacaccaa gttaaaagca tggtggtttg ctcatctttt cccctcccga aataccatgt | 4980 |
| agatggggtg cagaaggtaa agtgcgagaa ggttctcctg ttcgacccga cggtaccttc | 5040 |
| agtggttagt ccgcggaagt atgccgcatc tacgacggac cactcagatc ggtcgttacg | 5100 |
| agggtttgac ttggactgga ccaccgactc gtcttccact gccagcgata ccatgtcgct | 5160 |
| acccagtttg cagtcgtgtg acatcgactc gatctacgag ccaatggctc ccatagtagt | 5220 |
| gacggctgac gtacaccctg aacccgcagg catcgcggac ctggcggcag atgtgcaccc | 5280 |
| tgaacccgca gaccatgtgg acctcgagaa cccgattcct ccaccgcgcc cgaagagagc | 5340 |
| tgcatacctt gcctcccgcg cggcggagcg accggtgccg gcgccgagaa agccgacgcc | 5400 |
| tgccccaagg actgcgttta ggaacaagct gcctttgacg ttcggcgact ttgacgagca | 5460 |
| cgaggtcgat gcgttggcct ccgggattac tttcggagac ttcgacgacg tcctgcgact | 5520 |
| aggccgcgcg ggtgcatata ttttctcctc ggacactggc agcggacatt tacaacaaaa | 5580 |
| atccgttagg cagcacaatc tccagtgcgc acaactggat gcggtccagg aggagaaaat | 5640 |
| gtacccgcca aaattggata ctgagaggga gaagctgttg ctgctgaaaa tgcagatgca | 5700 |
| cccatcggag gctaataaga gtcgatacca gtctcgcaaa gtggagaaca tgaaagccac | 5760 |
| ggtggtggac aggctcacat cgggggccag attgtacacg ggagcggacg taggccgcat | 5820 |
| accaacatac gcggttcggt acccccgccc cgtgtactcc cctaccgtga tcgaaagatt | 5880 |
| ctcaagcccc gatgtagcaa tcgcagcgtg caacgaatac ctatccagaa attacccaac | 5940 |
| agtggcgtcg taccagataa cagatgaata cgacgcatac ttggacatgg ttgacgggtc | 6000 |
| ggatagttgc ttggacagag cgacattctg cccggcgaag ctccggtgct acccgaaaca | 6060 |
| tcatgcgtac caccagccga ctgtacgcag tgccgtcccg tcaccctttc agaacacact | 6120 |
| acagaacgtg ctagcggccg ccaccaagag aaactgcaac gtcacgcaaa tgcgagaact | 6180 |
| acccaccatg gactcggcag tgttcaacgt ggagtgcttc aagcgctatg cctgctccgg | 6240 |
| agaatattgg gaagaatatg ctaaacaacc tatccggata accactgaga acatcactac | 6300 |
| ctatgtgacc aaattgaaag gcccgaaagc tgctgccttg ttcgctaaga cccacaactt | 6360 |
| ggttccgctg caggaggttc ccatggacag attcacggtc gacatgaaac gagatgtcaa | 6420 |
| agtcactcca gggacgaaac acacagagga aagacccaaa gtccaggtaa ttcaagcagc | 6480 |
| ggagccattg gcgaccgctt acctgtgcgg catccacagg gaattagtaa ggagactaaa | 6540 |
| tgctgtgtta cgccctaacg tgcacacatt gtttgatatg tcggccgaag actttgacgc | 6600 |
| gatcatcgcc tctcacttcc acccaggaga cccggttcta gagacggaca ttgcatcatt | 6660 |
| cgacaaaagc caggacgact ccttggctct tacaggttta atgatcctcg aagatctagg | 6720 |
| ggtggatcag tacctgctgg acttgatcga ggcagccttt ggggaaatat ccagctgtca | 6780 |
| cctaccaact ggcacgcgct tcaagttcgg agctatgatg aaatcgggca tgtttctgac | 6840 |
| tttgtttatt aacactgttt tgaacatcac catagcaagc agggtactgg agcagagact | 6900 |
| cactgactcc gcctgtgcgg ccttcatcgg cgacgacaac atcgttcacg gagtgatctc | 6960 |
| cgacaagctg atggcggaga ggtgcgcgtc gtgggtcaac atggaggtga agatcattga | 7020 |
| cgctgtcatg ggcgaaaaac ccccatattt ttgtggggga ttcatagttt ttgacagcgt | 7080 |
| cacacagacc gcctgccgtg tttcagaccc acttaagcgc ctgttcaagt tgggtaagcc | 7140 |
| gctaacagct gaagacaagc aggacgaaga caggcgacga gcactgagtg acgaggttag | 7200 |
| caagtggttc cggacaggct tgggggccga actggaggtg gcactaacat ctaggtatga | 7260 |
| ggtagagggc tgcaaaagta tcctcatagc catggccacc ttggcgaggg acattaaggc | 7320 |
| gtttaagaaa ttgagaggac ctgttataca cctctacggc ggtcctagat tggtgcgtta | 7380 |
| atacacagaa ttctgattgg atcccaaacg ggccctctag actcgagcgg ccgccactgt | 7440 |
| gctggatatc tgcagaattc caccacactg gactagtgga tctatggcgt acccatacga | 7500 |
| tgttccagat tacgctagct tgagatctac catgtctcag agcaaccggg agctggtggt | 7560 |
| tgactttctc tcctacaagc tttcccagaa aggatacagc tggagtcagt ttagtgatgt | 7620 |
| ggaagagaac aggactgagg ccccagaagg gactgaatcg gagatggaga cccccagtgc | 7680 |
| catcaatggc aacccatcct ggcacctggc agacagcccc gcggtgaatg gagccactgc | 7740 |
| gcacagcagc agtttggatg cccgggaggt gatccccatg gcagcagtaa agcaagcgct | 7800 |
| gagggaggca ggcgacgagt ttgaactgcg gtaccggcgg gcattcagtg acctgacatc | 7860 |
| ccagctccac atcaccccag ggacagcata tcagagcttt gaacaggtag tgaatgaact | 7920 |
| cttccgggat ggggtaaact ggggtcgcat tgtggccttt ttctccttcg gcggggcact | 7980 |
| gtgcgtggaa agcgtagaca aggagatgca ggtattggtg agtcggatcg cagcttggat | 8040 |
| ggccacttac ctgaatgacc acctagagcc ttggatccag gagaacggcg gctgggatac | 8100 |
| ttttgtggaa ctctatggga acaatgcagc agccgagagc cgaaagggcc aggaacgctt | 8160 |
| caaccgctgg ttcctgacgg gcatgactgt ggccggcatg gttctactgg gctcactctt | 8220 |
| cagtcggaaa tgaagatccg agctcggtac caagcttaag tttgggtaat taattgaatt | 8280 |
| acatccctac gcaaacgttt tacggccgcc ggtggcgccc gcgcccggcg gcccgtcctt | 8340 |
| ggccgttgca ggccactccg gtggctcccg tcgtccccga cttccaggcc cagcagatgc | 8400 |
| agcaactcat cagcgccgta aatgcgctga caatgagaca gaacgcaatt gctcctgcta | 8460 |
| ggcctcccaa accaaagaag aagaagacaa ccaaaccaaa gccgaaaacg cagcccaaga | 8520 |
| agatcaacgg aaaaacgcag cagcaaaaga agaaagacaa gcaagccgac aagaagaaga | 8580 |
| agaaacccgg aaaaagagaa agaatgtgca tgaagattga aaatgactgt atcttcgtat | 8640 |
| gcggctagcc acagtaacgt agtgtttcca gacatgtcgg gcaccgcact atcatgggtg | 8700 |
| cagaaaatct cgggtggtct gggggccttc gcaatcggcg ctatcctggt gctggttgtg | 8760 |
| gtcacttgca ttgggctccg cagataagtt agggtaggca atggcattga tatagcaaga | 8820 |
| aaattgaaaa cagaaaaagt tagggtaagc aatggcatat aaccataact gtataacttg | 8880 |
| taacaaagcg caacaagacc tgcgcaattg gccccgtggt ccgcctcacg gaaactcggg | 8940 |
| gcaactcata ttgacacatt aattggcaat aattggaagc ttacataagc ttaattcgac | 9000 |
| gaataattgg atttttattt tattttgcaa ttggttttta atatttccaa aaaaaaaaaa | 9060 |
| aaaaaaaaaa aaaaaaaaaa aaaaaaaaaa aaaaaaaaaa aaaaaaaaaa aaaaaaaact | 9120 |
| agtgatcata atcagccata ccacatttgt agaggtttta cttgctttaa aaaacctccc | 9180 |
| acacctcccc ctgaacctga aacataaaat gaatgcaatt gttgttgtta acttgtttat | 9240 |
| tgcagcttat aatggttaca aataaagcaa tagcatcaca aatttcacaa ataaagcatt | 9300 |
| tttttcactg cattctagtt gtggtttgtc caaactcatc aatgtatctt atcatgtctg | 9360 |
| gatctagtct gcattaatga atcggccaac gcgcggggag aggcggtttg cgtattgggc | 9420 |
| gctcttccgc ttcctcgctc actgactcgc tgcgctcggt cgttcggctg cggcgagcgg | 9480 |
| tatcagctca ctcaaaggcg gtaatacggt tatccacaga atcaggggat aacgcaggaa | 9540 |
| agaacatgtg agcaaaaggc cagcaaaagg ccaggaaccg taaaaaggcc gcgttgctgg | 9600 |
| cgtttttcca taggctccgc ccccctgacg agcatcacaa aaatcgacgc tcaagtcaga | 9660 |
| ggtggcgaaa cccgacagga ctataaagat accaggcgtt tccccctgga agctccctcg | 9720 |
| tgcgctctcc tgttccgacc ctgccgctta ccggatacct gtccgccttt ctcccttcgg | 9780 |
| gaagcgtggc gctttctcaa tgctcgcgct gtaggtatct cagttcggtg taggtcgttc | 9840 |
| gctccaagct gggctgtgtg cacgaacccc ccgttcagcc cgaccgctgc gccttatccg | 9900 |
| gtaactatcg tcttgagtcc aacccggtaa gacacgactt atcgccactg gcagcagcca | 9960 |
| ctggtaacag gattagcaga gcgaggtatg taggcggtgc tacagagttc ttgaagtggt | 10020 |
| ggcctaacta cggctacact agaaggacag tatttggtat ctgcgctctg ctgaagccag | 10080 |
| ttaccttcgg aaaaagagtt ggtagctctt gatccggcaa acaaaccacc gctggtagcg | 10140 |
| gtggtttttt tgtttgcaag cagcagatta cgcgcagaaa aaaaggatct caagaagatc | 10200 |
| ctttgatctt ttctacgggg cattctgacg ctcagtggaa cgaaaactca cgttaaggga | 10260 |
| ttttggtcat gagattatca aaaaggatct tcacctagat ccttttaaat taaaaatgaa | 10320 |
| gttttaaatc aatctaaagt atatatgagt aaacttggtc tgacagttac caatgcttaa | 10380 |
| tcagtgaggc acctatctca gcgatctgtc tatttcgttc atccatagtt gcctgactcc | 10440 |
| ccgtcgtgta gataactacg atacgggagg gcttaccatc tggccccagt gctgcaatga | 10500 |
| taccgcgaga cccacgctca ccggctccag atttatcagc aataaaccag ccagccggaa | 10560 |
| gggccgagcg cagaagtggt cctgcaactt tatccgcctc catccagtct attaattgtt | 10620 |
| gccgggaagc tagagtaagt agttcgccag ttaatagttt gcgcaacgtt gttgccattg | 10680 |
| ctacaggcat cgtggtgtca cgctcgtcgt ttggtatggc ttcattcagc tccggttccc | 10740 |
| aacgatcaag gcgagttaca tgatccccca tgttgtgcaa aaaagcggtt agctccttcg | 10800 |
| gtcctccgat cgttgtcaga agtaagttgg ccgcagtgtt atcactcatg gttatggcag | 10860 |
| cactgcataa ttctcttact gtcatgccat ccgtaagatg cttttctgtg actggtgagt | 10920 |
| actcaaccaa gtcattctga gaatagtgta tgcggcgacc gagttgctct tgcccggcgt | 10980 |
| caatacggga taataccgcg ccacatagca gaactttaaa agtgctcatc attggaaaac | 11040 |
| gttcttcggg gcgaaaactc tcaaggatct taccgctgtt gagatccagt tcgatgtaac | 11100 |
| ccactcgtgc acccaactga tcttcagcat cttttacttt caccagcgtt tctgggtgag | 11160 |
| caaaaacagg aaggcaaaat gccgcaaaaa agggaataag ggcgacacgg aaatgttgaa | 11220 |
| tactcatact cttccttttt caatattatt gaagcattta tcagggttat tgtctcatga | 11280 |
| gcggatacat atttgaatgt atttagaaaa ataaacaaat aggggttccg cgcacatttc | 11340 |
| cccgaaaagt gccacctgac gtctaagaaa ccattattat catgacatta acctataaaa | 11400 |
| ataggcgtat cacgaggccc tttcgtctcg cgcgtttcgg tgatgacggt gaaaacctct | 11460 |
| gacacatgca gctcccggag acggtcacag cttctgtcta agcggatgcc gggagcagac | 11520 |
| aagcccgtca gggcgcgtca gcgggtgttg gcgggtgtcg gggctggctt aactatgcgg | 11580 |
| catcagagca gattgtactg agagtgcacc atatcgacgc tctcccttat gcgactcctg | 11640 |
| cattaggaag cagcccagta ctaggttgag gccgttgagc accgccgccg caaggaatgg | 11700 |
| tgcatgcgta atcaattacg gggtcattag ttcatagccc atatatggag ttccgcgtta | 11760 |
| cataacttac ggtaaatggc ccgcctggct gaccgcccaa cgacccccgc ccattgacgt | 11820 |
| caataatgac gtatgttccc atagtaacgc caatagggac tttccattga cgtcaatggg | 11880 |
| tggagtattt acggtaaact gcccacttgg cagtacatca agtgtatcat atgccaagta | 11940 |
| cgccccctat tgacgtcaat gacggtaaat ggcccgcctg gcattatgcc cagtacatga | 12000 |
| ccttatggga ctttcctact tggcagtaca tctacgtatt agtcatcgct attaccatgg | 12060 |
| tgatgcggtt ttggcagtac atcaatgggc gtggatagcg gtttgactca cggggatttc | 12120 |
| caagtctcca ccccattgac gtcaatggga gtttgttttg gcaccaaaat caacgggact | 12180 |
| ttccaaaatg tcgtaacaac tccgccccat tgacgcaaat gggcggtagg cgtgtacggt | 12240 |
| gggaggtcta tataagcaga gctctctggc taactagaga acccactgct taactggctt | 12300 |
| atcgaaatta atacgactca ctatagggag accggaagct tgaattc | 12347 |

| | |
|---|---|
| SEQ ID NO: 66 | |
| atggcggatg tgtgacatac acgacgccaa aagattttgt tccagctcct gccacctccg | 60 |
| ctacgcgaga gattaaccac ccacgatggc cgccaaagtg catgttgata ttgaggctga | 120 |
| cagcccattc atcaagtctt tgcagaaggc atttccgtcg ttcgaggtgg agtcattgca | 180 |
| ggtcacacca aatgaccatg caaatgccag agcattttcg cacctggcta ccaaattgat | 240 |
| cgagcaggag actgacaaag acacactcat cttggatatc ggcagtgcgc cttccaggag | 300 |
| aatgatgtct acgcacaaat accactgcgt atgccctatg cgcagcgcag aagaccccga | 360 |
| aaggctcgat agctacgcaa agaaactggc agcggcctcc gggaaggtgc tggatagaga | 420 |
| gatcgcagga aaaatcaccg acctgcagac cgtcatggct acgccagacg ctgaatctcc | 480 |
| taccttttgc ctgcatacag acgtcacgtg tcgtacggca gccgaagtgg ccgtatacca | 540 |
| ggacgtgtat gctgtacatg caccaacatc gctgtaccat caggcgatga aaggtgtcag | 600 |
| aacggcgtat tggattgggt ttgacaccac cccgtttatg tttgacgcgc tagcaggcgc | 660 |
| gtatccaacc tacgccacaa actgggccga cgagcaggtg ttacaggcca ggaacatagg | 720 |
| actgtgtgca gcatccttga ctgagggaag actcggcaaa ctgtccattc tccgcaagaa | 780 |
| gcaattgaaa ccttgcgaca cagtcatgtt ctcggtagga tctacattgt acactgagag | 840 |
| cagaaagcta ctgaggagct ggcacttacc ctccgtattc cacctgaaag gtaaacaatc | 900 |
| ctttacctgt aggtgcgata ccatcgtatc atgtgaaggg tacgtagtta agaaaatcac | 960 |
| tatgtgcccc ggcctgtacg gtaaaacggt agggtacgcc gtgacgtatc acgcggaggg | 1020 |
| attcctagtg tgcaagacca cagacactgt caaaggagaa agagtctcat tccctgtatg | 1080 |
| cacctacgtc ccctcaacca tctgtgatca aatgactggc atactagcga ccgacgtcac | 1140 |
| accggaggac gcacagaagt tgttagtggg attgaatcag aggatagttg tgaacggaag | 1200 |
| aacacagcga aacactaaca cgatgaagaa ctatctgctt ccgattgtgg ccgtcgcatt | 1260 |
| tagcaagtgg gcgagggaat acaaggcaga ccttgatgat gaaaaacctc tgggtgtccg | 1320 |
| agagaggtca cttacttgct gctgcttgtg ggcatttaaa acgaggaaga tgcacaccat | 1380 |
| gtacaagaaa ccagacaccc agacaatagt gaaggtgcct tcagagttta actcgttcgt | 1440 |
| catcccgagc ctatggtcta caggcctcgc aatcccagtc agatcacgca ttaagatgct | 1500 |
| tttggccaag aagaccaagc gagagttaat acctgttctc gacgcgtcgt cagccaggga | 1560 |
| tgctgaacaa gaggagaagg agaggttgga ggccgagctg actagagaag ccttaccacc | 1620 |
| cctcgtcccc atcgcgccgg cggagacggg agtcgtcgac gtcgacgttg aagaactaga | 1680 |
| gtatcacgca ggtgcagggg tcgtggaaac acctcgcagc gcgttgaaag tcaccgcaca | 1740 |
| gccgaacgac gtactactag gaaattacgt agttctgtcc ccgcagaccg tgctcaagag | 1800 |
| ctccaagttg gcccccgtgc accctctagc agagcaggtg aaaataataa cacataacgg | 1860 |
| gagggccggc ggttaccagg tcgacggata tgacggcagg gtcctactac catgtggatc | 1920 |
| ggccattccg gtccctgagt ttcaagcttt gagcgagagc gccactatgg tgtacaacga | 1980 |
| aagggagttc gtcaacagga aactatacca tattgccgtt cacggaccgt cgctgaacac | 2040 |
| cgacgaggag aactacgaga aagtcagagc tgaaagaact gacgccgagt acgtgttcga | 2100 |
| cgtagataaa aaatgctgcg tcaagagaga ggaagcgtcg ggtttggtgt tggtgggaga | 2160 |
| gctaaccaac cccccgttcc atgaattcgc ctacgaaggg ctgaagatca ggccgtcggc | 2220 |
| accatataag actacagtag taggagtctt tggggttccg ggatcaggca agtctgctat | 2280 |
| tattaagagc ctcgtgacca aacacgatct ggtcaccagc ggcaagaagg agaactgcca | 2340 |
| ggaaatagtt aacgacgtga agaagcaccg cgggaagggg acaagtaggg aaaacagtga | 2400 |
| ctccatcctg ctaaacgggt gtcgtcgtgc cgtggacatc ctatatgtgg acgaggcttt | 2460 |
| cgctagccat tccggtactc tgctggccct aattgctctt gttaaacctc ggagcaaagt | 2520 |
| ggtgttatgc ggagacccca agcaatgcgg attcttcaat atgatgcagc ttaaggtgaa | 2580 |
| cttcaaccac aacatctgca ctgaagtatg tcataaaagt atatccagac gttgcacgcg | 2640 |
| tccagtcacg gccatcgtgt ctacgttgca ctacggaggc aagatgcgca cgaccaaccc | 2700 |
| gtgcaacaaa cccataatca tagacaccac aggacagacc aagcccaagc caggagacat | 2760 |
| cgtgttaaca tgcttccgag gctgggcaaa gcagctgcag ttggactacc gtggacacga | 2820 |
| agtcatgaca gcagcagcat ctcagggcct cacccgcaaa ggggtatacg ccgtaaggca | 2880 |
| gaaggtgaat gaaaatccct tgtatgcccc tgcgtcggag cacgtgaatg tactgctgac | 2940 |
| gcgcactgag gataggctgg tgtggaaaac gctggccggc gatccctgga ttaaggtcct | 3000 |
| atcaaacatt ccacagggta actttacggc cacattggaa gaatggcaag aagaacacga | 3060 |
| caaaataatg aaggtgattg aaggaccggc tgcgcctgtg gacgcgttcc agaacaaagc | 3120 |
| gaacgtgtgt tgggcgaaaa gcctggtgcc tgtcctggac actgccggaa tcagattgac | 3180 |
| agcagaggag tggagcacca taattacagc atttaaggag gacagagctt actctccagt | 3240 |
| ggtggccttg aatgaaattt gcaccaagta ctatggagtt gacctggaca gtggcctgtt | 3300 |
| ttctgccccg aaggtgtccc tgtattacga gaacaaccac tgggataaca gacctggtgg | 3360 |
| aaggatgtat ggattcaatg ccgcaacagc tgccaggctg gaagctagac ataccttcct | 3420 |
| gaaggggcag tggcatacgg gcaagcaggc agttatcgca gaaagaaaaa tccaaccgct | 3480 |
| ttctgtgctg gacaatgtaa ttcctatcaa ccgcaggctg ccgcacgccc tggtggctga | 3540 |
| gtacaagacg gttaaaggca gtagggttga gtggctggtc aataaagtaa gagggtacca | 3600 |
| cgtcctgctg gtgagtgagt acaacctggc tttgcctcga cgcagggtca cttggttgtc | 3660 |
| accgctgaat gtcacaggcg ccgataggtg ctacgaccta agtttaggac tgccggctga | 3720 |
| cgccggcagg ttcgacttgg tctttgtgaa cattcacacg gaattcagaa tccaccacta | 3780 |
| ccagcagtgt gtcgaccacg ccatgaagct gcagatgctt gggggagatg cgctacgact | 3840 |
| gctaaaaccc ggcggcatct tgatgagagc ttacggatac gccgataaaa tcagcgaagc | 3900 |
| cgttgtttcc tccttaagca gaaagttctc gtctgcaaga gtgttgcgcc cggattgtgt | 3960 |
| caccagcaat acagaagtgt tcttgctgtt ctccaacttt gacaacggaa agagaccctc | 4020 |
| tacgctacac cagatgaata ccaagctgag tgccgtgtat gccggagaag ccatgcacac | 4080 |
| ggccgggtgt gcaccatcct acagagttaa gagagcagac atagccacgt gcacagaagc | 4140 |
| ggctgtggtt aacgcagcta acgcccgtgg aactgtaggg gatggcgtat gcagggccgt | 4200 |
| ggcgaagaaa tggccgtcag cctttaaggg agcagcaaca ccagtgggca caattaaaac | 4260 |
| agtcatgtgc ggctcgtacc ccgtcatcca cgctgtagcg cctaatttct ctgccacgac | 4320 |
| tgaagcggaa ggggaccgcg aattggccgc tgtctaccgg gcagtggccg ccgaagtaaa | 4380 |
| cagactgtca ctgagcagcg tagccatccc gctgctgtcc acaggagtgt tcagcggcgg | 4440 |
| aagagatagg ctgcagcaat ccctcaacca tctattcaca gcaatggacg ccacggacgc | 4500 |
| tgacgtgacc atctactgca gagacaaaag ttgggagaag aaaatccagg aagccattga | 4560 |
| catgaggacg gctgtggagt tgctcaatga tgacgtggag ctgaccacag acttggtgag | 4620 |
| agtgcacccg gacagcagcc tggtgggtcg taagggctac agtaccactg acgggtcgct | 4680 |
| gtactcgtac tttgaaggta cgaaattcaa ccaggctgct attgatatgg cagagatact | 4740 |
| gacgttgtgg cccagactgc aagaggcaaa cgaacagata tgcctatacg cgctgggcga | 4800 |
| aacaatggac aacatcagat ccaaatgtcc ggtgaacgat tccgattcat caacacctcc | 4860 |
| caggacagtg ccctgcctgt gccgctacgc aatgacagca gaacggatcg cccgccttag | 4920 |
| gtcacaccaa gttaaaagca tggtggtttg ctcatctttt cccctcccga aataccatgt | 4980 |
| agatggggtg cagaaggtaa agtgcgagaa ggttctcctg ttcgacccga cggtaccttc | 5040 |
| agtggttagt ccgcggaagt atgccgcatc tacgacggac cactcagatc ggtcgttacg | 5100 |
| agggtttgac ttggactgga ccaccgactc gtcttccact gccagcgata ccatgtcgct | 5160 |
| acccagtttg cagtcgtgtg acatcgactc gatctacgag ccaatggctc ccatagtagt | 5220 |
| gacggctgac gtacaccctg aacccgcagg catcgcggac ctggcggcag atgtgcaccc | 5280 |
| tgaacccgca gaccatgtgg acctcgagaa cccgattcct ccaccgcgcc cgaagagagc | 5340 |
| tgcatacctt gcctcccgcg cggcggagcg accggtgccg gcgccgagaa agccgacgcc | 5400 |
| tgccccaagg actgcgttta ggaacaagct gcctttgacg ttcggcgact ttgacgagca | 5460 |
| cgaggtcgat gcgttggcct ccgggattac tttcggagac ttcgacgacg tcctgcgact | 5520 |
| aggccgcgcg ggtgcatata ttttctcctc ggacactggc agcggacatt tacaacaaaa | 5580 |
| atccgttagg cagcacaatc tccagtgcgc acaactggat gcggtccagg aggagaaaat | 5640 |
| gtacccgcca aaattggata ctgagaggga gaagctgttg ctgctgaaaa tgcagatgca | 5700 |
| cccatcggag gctaataaga gtcgatacca gtctcgcaaa gtggagaaca tgaaagccac | 5760 |
| ggtggtggac aggctcacat cgggggccag attgtacacg ggagcggacg taggccgcat | 5820 |
| accaacatac gcggttcggt acccccgccc cgtgtactcc cctaccgtga tcgaaagatt | 5880 |
| ctcaagcccc gatgtagcaa tcgcagcgtg caacgaatac ctatccagaa attacccaac | 5940 |
| agtggcgtcg taccagataa cagatgaata cgacgcatac ttggacatgg ttgacgggtc | 6000 |
| ggatagttgc ttggacagag cgacattctg cccggcgaag ctccggtgct acccgaaaca | 6060 |
| tcatgcgtac caccagccga ctgtacgcag tgccgtcccg tcaccctttc agaacacact | 6120 |
| acagaacgtg ctagcggccg ccaccaagag aaactgcaac gtcacgcaaa tgcgagaact | 6180 |
| acccaccatg gactcggcag tgttcaacgt ggagtgcttc aagcgctatg cctgctccgg | 6240 |
| agaatattgg gaagaatatg ctaaacaacc tatccggata accactgaga acatcactac | 6300 |
| ctatgtgacc aaattgaaag gcccgaaagc tgctgccttg ttcgctaaga cccacaactt | 6360 |
| ggttccgctg caggaggttc ccatggacag attcacggtc gacatgaaac gagatgtcaa | 6420 |
| agtcactcca gggacgaaac acacagagga aagacccaaa gtccaggtaa ttcaagcagc | 6480 |
| ggagccattg gcgaccgctt acctgtgcgg catccacagg gaattagtaa ggagactaaa | 6540 |
| tgctgtgtta cgccctaacg tgcacacatt gtttgatatg tcggccgaag actttgacgc | 6600 |
| gatcatcgcc tctcacttcc acccaggaga cccggttcta gagacggaca ttgcatcatt | 6660 |
| cgacaaaagc caggacgact ccttggctct tacaggttta atgatcctcg aagatctagg | 6720 |
| ggtggatcag tacctgctgg acttgatcga ggcagccttt ggggaaatat ccagctgtca | 6780 |
| cctaccaact ggcacgcgct tcaagttcgg agctatgatg aaatcgggca tgtttctgac | 6840 |
| tttgtttatt aacactgttt tgaacatcac catagcaagc agggtactgg agcagagact | 6900 |
| cactgactcc gcctgtgcgg ccttcatcgg cgacgacaac atcgttcacg gagtgatctc | 6960 |
| cgacaagctg atggcggaga ggtgcgcgtc gtgggtcaac atggaggtga agatcattga | 7020 |
| cgctgtcatg ggcgaaaaac ccccatattt ttgtggggga ttcatagttt ttgacagcgt | 7080 |
| cacacagacc gcctgccgtg tttcagaccc acttaagcgc ctgttcaagt tgggtaagcc | 7140 |
| gctaacagct gaagacaagc aggacgaaga caggcgacga gcactgagtg acgaggttag | 7200 |
| caagtggttc cggacaggct tgggggccga actggaggtg gcactaacat ctaggtatga | 7260 |
| ggtagagggc tgcaaaagta tcctcatagc catggccacc ttggcgaggg acattaaggc | 7320 |
| gtttaagaaa ttgagaggac ctgttataca cctctacggc ggtcctagat tggtgcgtta | 7380 |
| atacacagaa ttctgattgg atcccaaacg ggccctctag actcgagcgg ccgccactgt | 7440 |
| gctggatatc tgcagaattc atgcatggag atacacctac attgcatgaa tatatgttag | 7500 |
| atttgcaacc agagacaact gatctctact gttatgagca attaaatgac agctcagagg | 7560 |
| aggaggatga aatagatggt ccagctggac aagcagaacc ggacagagcc cattacaata | 7620 |
| ttgtaacctt ttgttgcaag tgtgactcta cgcttcggtt gtgcgtacaa agcacacacg | 7680 |
| tagacattcg tactttggaa gacctgttaa tgggcacact aggaattgtg tgccccatct | 7740 |
| gttctcagaa accaggatct atggcgtacc catacgatgt tccagattac gctagcttga | 7800 |
| gatctaccat gtctcagagc aaccgggagc tggtggttga ctttctctcc tacaagcttt | 7860 |
| cccagaaagg atacagctgg agtcagttta gtgatgtgga agagaacagg actgaggccc | 7920 |
| cagaagggac tgaatcggag atggagaccc ccagtgccat caatggcaac ccatcctggc | 7980 |
| acctggcaga cagccccgcg gtgaatggag ccactgcgca cagcagcagt ttggatgccc | 8040 |
| gggaggtgat ccccatggca gcagtaaagc aagcgctgag ggaggcaggc gacgagtttg | 8100 |
| aactgcggta ccggcgggca ttcagtgacc tgacatccca gctccacatc accccaggga | 8160 |
| cagcatatca gagctttgaa caggtagtga atgaactctt ccgggatggg gtaaactggg | 8220 |
| gtcgcattgt ggcctttttc tccttcggcg gggcactgtg cgtggaaagc gtagacaagg | 8280 |
| agatgcaggt attggtgagt cggatcgcag cttggatggc cacttacctg aatgaccacc | 8340 |
| tagagccttg gatccaggag aacggcggct gggatacttt tgtggaactc tatgggaaca | 8400 |
| atgcagcagc cgagagccga aagggccagg aacgcttcaa ccgctggttc ctgacgggca | 8460 |
| tgactgtggc cggcgtggtt ctgctgggct cactcttcag tcggaaatga agatccaagc | 8520 |
| ttaagtttgg gtaattaatt gaattacatc cctacgcaaa cgttttacgg ccgccggtgg | 8580 |
| cgcccgcgcc cggcggcccg tccttggccg ttgcaggcca ctccggtggc tcccgtcgtc | 8640 |
| cccgacttcc aggcccagca gatgcagcaa ctcatcagcg ccgtaaatgc gctgacaatg | 8700 |
| agacagaacg caattgctcc tgctaggcct cccaaaccaa agaagaagaa gacaaccaaa | 8760 |
| ccaaagccga aaacgcagcc caagaagatc aacggaaaaa cgcagcagca aaagaagaaa | 8820 |
| gacaagcaag ccgacaagaa gaagaagaaa cccggaaaaa gagaaagaat gtgcatgaag | 8880 |
| attgaaaatg actgtatctt cgtatgcggc tagccacagt aacgtagtgt ttccagacat | 8940 |
| gtcgggcacc gcactatcat gggtgcagaa aatctcgggt ggtctggggg ccttcgcaat | 9000 |
| cggcgctatc ctggtgctgg ttgtggtcac ttgcattggg ctccgcagat aagttagggt | 9060 |
| aggcaatggc attgatatag caagaaaatt gaaaacagaa aaagttaggg taagcaatgg | 9120 |
| catataacca taactgtata acttgtaaca aagcgcaaca agacctgcgc aattggcccc | 9180 |
| gtggtccgcc tcacggaaac tcggggcaac tcatattgac acattaattg gcaataattg | 9240 |
| gaagcttaca taagcttaat tcgacgaata attggatttt tattttattt tgcaattggt | 9300 |
| ttttaatatt tccaaaaaaa aaaaaaaaaa aaaaaaaaaa aaaaaaaaaa aaaaaaaaaa | 9360 |
| aaaaaaaaaa aaaaaaaaaa aaactagtga tcataatcag ccataccaca tttgtagagg | 9420 |
| ttttacttgc tttaaaaaac ctcccacacc tccccctgaa cctgaaacat aaaatgaatg | 9480 |
| caattgttgt tgttaacttg tttattgcag cttataatgg ttacaaataa agcaatagca | 9540 |
| tcacaaattt cacaaataaa gcattttttt cactgcattc tagttgtggt ttgtccaaac | 9600 |
| tcatcaatgt atcttatcat gtctggatct agtctgcatt aatgaatcgg ccaacgcgcg | 9660 |
| gggagaggcg gtttgcgtat tgggcgctct tccgcttcct cgctcactga ctcgctgcgc | 9720 |
| tcggtcgttc ggctgcggcg agcggtatca gctcactcaa aggcggtaat acggttatcc | 9780 |
| acagaatcag gggataacgc aggaaagaac atgtgagcaa aaggccagca aaaggccagg | 9840 |
| aaccgtaaaa aggccgcgtt gctggcgttt ttccataggc tccgcccccc tgacgagcat | 9900 |
| cacaaaaatc gacgctcaag tcagaggtgg cgaaacccga caggactata aagataccag | 9960 |
| gcgtttcccc ctggaagctc cctcgtgcgc tctcctgttc cgaccctgcc gcttaccgga | 10020 |
| tacctgtccg cctttctccc ttcgggaagc gtggcgcttt ctcaatgctc gcgctgtagg | 10080 |
| tatctcagtt cggtgtaggt cgttcgctcc aagctgggct gtgtgcacga accccccgtt | 10140 |
| cagcccgacc gctgcgcctt atccggtaac tatcgtcttg agtccaaccc ggtaagacac | 10200 |
| gacttatcgc cactggcagc agccactggt aacaggatta gcagagcgag gtatgtaggc | 10260 |
| ggtgctacag agttcttgaa gtggtggcct aactacggct acactagaag gacagtattt | 10320 |
| ggtatctgcg ctctgctgaa gccagttacc ttcggaaaaa gagttggtag ctcttgatcc | 10380 |
| ggcaaacaaa ccaccgctgg tagcggtggt ttttttgttt gcaagcagca gattacgcgc | 10440 |
| agaaaaaaag gatctcaaga agatcctttg atcttttcta cggggcattc tgacgctcag | 10500 |
| tggaacgaaa actcacgtta agggattttg gtcatgagat tatcaaaaag gatcttcacc | 10560 |
| tagatccttt taaattaaaa atgaagtttt aaatcaatct aaagtatata tgagtaaact | 10620 |
| tggtctgaca gttaccaatg cttaatcagt gaggcaccta tctcagcgat ctgtctattt | 10680 |
| cgttcatcca tagttgcctg actccccgtc gtgtagataa ctacgatacg ggagggctta | 10740 |
| ccatctggcc ccagtgctgc aatgataccg cgagacccac gctcaccggc tccagattta | 10800 |
| tcagcaataa accagccagc cggaagggcc gagcgcagaa gtggtcctgc aactttatcc | 10860 |
| gcctccatcc agtctattaa ttgttgccgg gaagctagag taagtagttc gccagttaat | 10920 |
| agtttgcgca acgttgttgc cattgctaca ggcatcgtgg tgtcacgctc gtcgtttggt | 10980 |
| atggcttcat tcagctccgg ttcccaacga tcaaggcgag ttacatgatc ccccatgttg | 11040 |
| tgcaaaaaag cggttagctc cttcggtcct ccgatcgttg tcagaagtaa gttggccgca | 11100 |
| gtgttatcac tcatggttat ggcagcactg cataattctc ttactgtcat gccatccgta | 11160 |
| agatgctttt ctgtgactgg tgagtactca accaagtcat tctgagaata gtgtatgcgg | 11220 |
| cgaccgagtt gctcttgccc ggcgtcaata cgggataata ccgcgccaca tagcagaact | 11280 |
| ttaaaagtgc tcatcattgg aaaacgttct tcggggcgaa aactctcaag gatcttaccg | 11340 |
| ctgttgagat ccagttcgat gtaacccact cgtgcaccca actgatcttc agcatctttt | 11400 |
| actttcacca gcgtttctgg gtgagcaaaa acaggaaggc aaaatgccgc aaaaaaggga | 11460 |
| ataagggcga cacggaaatg ttgaatactc atactcttcc tttttcaata ttattgaagc | 11520 |
| atttatcagg gttattgtct catgagcgga tacatatttg aatgtattta gaaaaataaa | 11580 |
| caaatagggg ttccgcgcac atttccccga aaagtgccac ctgacgtcta agaaaccatt | 11640 |
| attatcatga cattaaccta taaaaatagg cgtatcacga ggccctttcg tctcgcgcgt | 11700 |
| ttcggtgatg acggtgaaaa cctctgacac atgcagctcc cggagacggt cacagcttct | 11760 |
| gtctaagcgg atgccgggag cagacaagcc cgtcagggcg cgtcagcggg tgttggcggg | 11820 |
| tgtcggggct ggcttaacta tgcggcatca gagcagattg tactgagagt gcaccatatc | 11880 |
| gacgctctcc cttatgcgac tcctgcatta ggaagcagcc cagtactagg ttgaggccgt | 11940 |
| tgagcaccgc cgccgcaagg aatggtgcat gcgtaatcaa ttacggggtc attagttcat | 12000 |
| agcccatata tggagttccg cgttacataa cttacggtaa atggcccgcc tggctgaccg | 12060 |
| cccaacgacc cccgcccatt gacgtcaata atgacgtatg ttcccatagt aacgccaata | 12120 |
| gggactttcc attgacgtca atgggtggag tatttacggt aaactgccca cttggcagta | 12180 |
| catcaagtgt atcatatgcc aagtacgccc cctattgacg tcaatgacgg taaatggccc | 12240 |
| gcctggcatt atgcccagta catgacctta tgggactttc ctacttggca gtacatctac | 12300 |
| gtattagtca tcgctattac catggtgatg cggttttggc agtacatcaa tgggcgtgga | 12360 |
| tagcggtttg actcacgggg atttccaagt ctccacccca ttgacgtcaa tgggagtttg | 12420 |
| ttttggcacc aaaatcaacg ggactttcca aaatgtcgta acaactccgc cccattgacg | 12480 |
| caaatgggcg gtaggcgtgt acggtgggag gtctatataa gcagagctct ctggctaact | 12540 |
| agagaaccca ctgcttaact ggcttatcga aattaatacg actcactata gggagaccgg | 12600 |
| aagcttgaat tc | 12612 |
| SEQ ID NO: 67 | |
| atggcggatg tgtgacatac acgacgccaa aagattttgt tccagctcct gccacctccg | 60 |
| ctacgcgaga gattaaccac ccacgatggc cgccaaagtg catgttgata ttgaggctga | 120 |
| cagcccattc atcaagtctt tgcagaaggc atttccgtcg ttcgaggtgg agtcattgca | 180 |
| ggtcacacca aatgaccatg caaatgccag agcattttcg cacctggcta ccaaattgat | 240 |
| cgagcaggag actgacaaag acacactcat cttggatatc ggcagtgcgc cttccaggag | 300 |
| aatgatgtct acgcacaaat accactgcgt atgccctatg cgcagcgcag aagaccccga | 360 |
| aaggctcgat agctacgcaa agaaactggc agcggcctcc gggaaggtgc tggatagaga | 420 |
| gatcgcagga aaaatcaccg acctgcagac cgtcatggct acgccagacg ctgaatctcc | 480 |
| taccttttgc ctgcatacag acgtcacgtg tcgtacggca gccgaagtgg ccgtatacca | 540 |
| ggacgtgtat gctgtacatg caccaacatc gctgtaccat caggcgatga aaggtgtcag | 600 |
| aacggcgtat tggattgggt ttgacaccac cccgtttatg tttgacgcgc tagcaggcgc | 660 |
| gtatccaacc tacgccacaa actgggccga cgagcaggtg ttacaggcca ggaacatagg | 720 |
| actgtgtgca gcatccttga ctgagggaag actcggcaaa ctgtccattc tccgcaagaa | 780 |
| gcaattgaaa ccttgcgaca cagtcatgtt ctcggtagga tctacattgt acactgagag | 840 |
| cagaaagcta ctgaggagct ggcacttacc ctccgtattc cacctgaaag gtaaacaatc | 900 |
| ctttacctgt aggtgcgata ccatcgtatc atgtgaaggg tacgtagtta agaaaatcac | 960 |
| tatgtgcccc ggcctgtacg gtaaaacggt agggtacgcc gtgacgtatc acgcggaggg | 1020 |
| attcctagtg tgcaagacca cagacactgt caaaggagaa agagtctcat tccctgtatg | 1080 |
| cacctacgtc ccctcaacca tctgtgatca aatgactggc atactagcga ccgacgtcac | 1140 |
| accggaggac gcacagaagt tgttagtggg attgaatcag aggatagttg tgaacggaag | 1200 |
| aacacagcga aacactaaca cgatgaagaa ctatctgctt ccgattgtgg ccgtcgcatt | 1260 |
| tagcaagtgg gcgagggaat acaaggcaga ccttgatgat gaaaaacctc tgggtgtccg | 1320 |
| agagaggtca cttacttgct gctgcttgtg ggcatttaaa acgaggaaga tgcacaccat | 1380 |
| gtacaagaaa ccagacaccc agacaatagt gaaggtgcct tcagagttta actcgttcgt | 1440 |
| catcccgagc ctatggtcta caggcctcgc aatcccagtc agatcacgca ttaagatgct | 1500 |
| tttggccaag aagaccaagc gagagttaat acctgttctc gacgcgtcgt cagccaggga | 1560 |
| tgctgaacaa gaggagaagg agaggttgga ggccgagctg actagagaag ccttaccacc | 1620 |
| cctcgtcccc atcgcgccgg cggagacggg agtcgtcgac gtcgacgttg aagaactaga | 1680 |
| gtatcacgca ggtgcagggg tcgtggaaac acctcgcagc gcgttgaaag tcaccgcaca | 1740 |
| gccgaacgac gtactactag gaaattacgt agttctgtcc ccgcagaccg tgctcaagag | 1800 |
| ctccaagttg gcccccgtgc accctctagc agagcaggtg aaaataataa cacataacgg | 1860 |
| gagggccggc ggttaccagg tcgacggata tgacggcagg gtcctactac catgtggatc | 1920 |
| ggccattccg gtccctgagt ttcaagcttt gagcgagagc gccactatgg tgtacaacga | 1980 |
| aagggagttc gtcaacagga aactatacca tattgccgtt cacggaccgt cgctgaacac | 2040 |
| cgacgaggag aactacgaga aagtcagagc tgaaagaact gacgccgagt acgtgttcga | 2100 |
| cgtagataaa aaatgctgcg tcaagagaga ggaagcgtcg ggtttggtgt tggtgggaga | 2160 |
| gctaaccaac cccccgttcc atgaattcgc ctacgaaggg ctgaagatca ggccgtcggc | 2220 |
| accatataag actacagtag taggagtctt tggggttccg ggatcaggca agtctgctat | 2280 |
| tattaagagc ctcgtgacca aacacgatct ggtcaccagc ggcaagaagg agaactgcca | 2340 |
| ggaaatagtt aacgacgtga agaagcaccg cgggaagggg acaagtaggg aaaacagtga | 2400 |
| ctccatcctg ctaaacgggt gtcgtcgtgc cgtggacatc ctatatgtgg acgaggcttt | 2460 |
| cgctagccat tccggtactc tgctggccct aattgctctt gttaaacctc ggagcaaagt | 2520 |
| ggtgttatgc ggagacccca agcaatgcgg attcttcaat atgatgcagc ttaaggtgaa | 2580 |
| cttcaaccac aacatctgca ctgaagtatg tcataaaagt atatccagac gttgcacgcg | 2640 |
| tccagtcacg gccatcgtgt ctacgttgca ctacggaggc aagatgcgca cgaccaaccc | 2700 |
| gtgcaacaaa cccataatca tagacaccac aggacagacc aagcccaagc caggagacat | 2760 |
| cgtgttaaca tgcttccgag gctgggcaaa gcagctgcag ttggactacc gtggacacga | 2820 |
| agtcatgaca gcagcagcat ctcagggcct cacccgcaaa ggggtatacg ccgtaaggca | 2880 |
| gaaggtgaat gaaaatccct tgtatgcccc tgcgtcggag cacgtgaatg tactgctgac | 2940 |
| gcgcactgag gataggctgg tgtggaaaac gctggccggc gatccctgga ttaaggtcct | 3000 |
| atcaaacatt ccacagggta actttacggc cacattggaa gaatggcaag aagaacacga | 3060 |
| caaaataatg aaggtgattg aaggaccggc tgcgcctgtg gacgcgttcc agaacaaagc | 3120 |
| gaacgtgtgt tgggcgaaaa gcctggtgcc tgtcctggac actgccggaa tcagattgac | 3180 |
| agcagaggag tggagcacca taattacagc atttaaggag gacagagctt actctccagt | 3240 |
| ggtggccttg aatgaaattt gcaccaagta ctatggagtt gacctggaca gtggcctgtt | 3300 |
| ttctgccccg aaggtgtccc tgtattacga gaacaaccac tgggataaca gacctggtgg | 3360 |
| aaggatgtat ggattcaatg ccgcaacagc tgccaggctg gaagctagac ataccttcct | 3420 |
| gaaggggcag tggcatacgg gcaagcaggc agttatcgca gaaagaaaaa tccaaccgct | 3480 |
| ttctgtgctg gacaatgtaa ttcctatcaa ccgcaggctg ccgcacgccc tggtggctga | 3540 |
| gtacaagacg gttaaaggca gtagggttga gtggctggtc aataaagtaa gagggtacca | 3600 |
| cgtcctgctg gtgagtgagt acaacctggc tttgcctcga cgcagggtca cttggttgtc | 3660 |
| accgctgaat gtcacaggcg ccgataggtg ctacgaccta agtttaggac tgccggctga | 3720 |
| cgccggcagg ttcgacttgg tctttgtgaa cattcacacg gaattcagaa tccaccacta | 3780 |
| ccagcagtgt gtcgaccacg ccatgaagct gcagatgctt gggggagatg cgctacgact | 3840 |
| gctaaaaccc ggcggcatct tgatgagagc ttacggatac gccgataaaa tcagcgaagc | 3900 |
| cgttgtttcc tccttaagca gaaagttctc gtctgcaaga gtgttgcgcc cggattgtgt | 3960 |
| caccagcaat acagaagtgt tcttgctgtt ctccaacttt gacaacggaa agagaccctc | 4020 |
| tacgctacac cagatgaata ccaagctgag tgccgtgtat gccggagaag ccatgcacac | 4080 |
| ggccgggtgt gcaccatcct acagagttaa gagagcagac atagccacgt gcacagaagc | 4140 |
| ggctgtggtt aacgcagcta acgcccgtgg aactgtaggg gatggcgtat gcagggccgt | 4200 |
| ggcgaagaaa tggccgtcag cctttaaggg agcagcaaca ccagtgggca caattaaaac | 4260 |
| agtcatgtgc ggctcgtacc ccgtcatcca cgctgtagcg cctaatttct ctgccacgac | 4320 |
| tgaagcggaa ggggaccgcg aattggccgc tgtctaccgg gcagtggccg ccgaagtaaa | 4380 |
| cagactgtca ctgagcagcg tagccatccc gctgctgtcc acaggagtgt tcagcggcgg | 4440 |
| aagagatagg ctgcagcaat ccctcaacca tctattcaca gcaatggacg ccacggacgc | 4500 |
| tgacgtgacc atctactgca gagacaaaag ttgggagaag aaaatccagg aagccattga | 4560 |
| catgaggacg gctgtggagt tgctcaatga tgacgtggag ctgaccacag acttggtgag | 4620 |
| agtgcacccg gacagcagcc tggtgggtcg taagggctac agtaccactg acgggtcgct | 4680 |
| gtactcgtac tttgaaggta cgaaattcaa ccaggctgct attgatatgg cagagatact | 4740 |
| gacgttgtgg cccagactgc aagaggcaaa cgaacagata tgcctatacg cgctgggcga | 4800 |
| aacaatggac aacatcagat ccaaatgtcc ggtgaacgat tccgattcat caacacctcc | 4860 |
| caggacagtg ccctgcctgt gccgctacgc aatgacagca gaacggatcg cccgccttag | 4920 |
| gtcacaccaa gttaaaagca tggtggtttg ctcatctttt cccctcccga aataccatgt | 4980 |
| agatggggtg cagaaggtaa agtgcgagaa ggttctcctg ttcgacccga cggtaccttc | 5040 |
| agtggttagt ccgcggaagt atgccgcatc tacgacggac cactcagatc ggtcgttacg | 5100 |
| agggtttgac ttggactgga ccaccgactc gtcttccact gccagcgata ccatgtcgct | 5160 |
| acccagtttg cagtcgtgtg acatcgactc gatctacgag ccaatggctc ccatagtagt | 5220 |
| gacggctgac gtacaccctg aacccgcagg catcgcggac ctggcggcag atgtgcaccc | 5280 |
| tgaacccgca gaccatgtgg acctcgagaa cccgattcct ccaccgcgcc cgaagagagc | 5340 |
| tgcatacctt gcctcccgcg cggcggagcg accggtgccg gcgccgagaa agccgacgcc | 5400 |
| tgccccaagg actgcgttta ggaacaagct gcctttgacg ttcggcgact ttgacgagca | 5460 |
| cgaggtcgat gcgttggcct ccgggattac tttcggagac ttcgacgacg tcctgcgact | 5520 |
| aggccgcgcg ggtgcatata ttttctcctc ggacactggc agcggacatt tacaacaaaa | 5580 |
| atccgttagg cagcacaatc tccagtgcgc acaactggat gcggtccagg aggagaaaat | 5640 |
| gtacccgcca aaattggata ctgagaggga gaagctgttg ctgctgaaaa tgcagatgca | 5700 |
| cccatcggag gctaataaga gtcgatacca gtctcgcaaa gtggagaaca tgaaagccac | 5760 |
| ggtggtggac aggctcacat cgggggccag attgtacacg ggagcggacg taggccgcat | 5820 |
| accaacatac gcggttcggt acccccgccc cgtgtactcc cctaccgtga tcgaaagatt | 5880 |
| ctcaagcccc gatgtagcaa tcgcagcgtg caacgaatac ctatccagaa attacccaac | 5940 |
| agtggcgtcg taccagataa cagatgaata cgacgcatac ttggacatgg ttgacgggtc | 6000 |
| ggatagttgc ttggacagag cgacattctg cccggcgaag ctccggtgct acccgaaaca | 6060 |
| tcatgcgtac caccagccga ctgtacgcag tgccgtcccg tcaccctttc agaacacact | 6120 |
| acagaacgtg ctagcggccg ccaccaagag aaactgcaac gtcacgcaaa tgcgagaact | 6180 |
| acccaccatg gactcggcag tgttcaacgt ggagtgcttc aagcgctatg cctgctccgg | 6240 |
| agaatattgg gaagaatatg ctaaacaacc tatccggata accactgaga acatcactac | 6300 |
| ctatgtgacc aaattgaaag gcccgaaagc tgctgccttg ttcgctaaga cccacaactt | 6360 |
| ggttccgctg caggaggttc ccatggacag attcacggtc gacatgaaac gagatgtcaa | 6420 |
| agtcactcca gggacgaaac acacagagga aagacccaaa gtccaggtaa ttcaagcagc | 6480 |
| ggagccattg gcgaccgctt acctgtgcgg catccacagg gaattagtaa ggagactaaa | 6540 |
| tgctgtgtta cgccctaacg tgcacacatt gtttgatatg tcggccgaag actttgacgc | 6600 |
| gatcatcgcc tctcacttcc acccaggaga cccggttcta gagacggaca ttgcatcatt | 6660 |
| cgacaaaagc caggacgact ccttggctct tacaggttta atgatcctcg aagatctagg | 6720 |
| ggtggatcag tacctgctgg acttgatcga ggcagccttt ggggaaatat ccagctgtca | 6780 |
| cctaccaact ggcacgcgct tcaagttcgg agctatgatg aaatcgggca tgtttctgac | 6840 |
| tttgtttatt aacactgttt tgaacatcac catagcaagc agggtactgg agcagagact | 6900 |
| cactgactcc gcctgtgcgg ccttcatcgg cgacgacaac atcgttcacg gagtgatctc | 6960 |
| cgacaagctg atggcggaga ggtgcgcgtc gtgggtcaac atggaggtga agatcattga | 7020 |
| cgctgtcatg ggcgaaaaac ccccatattt ttgtggggga ttcatagttt ttgacagcgt | 7080 |
| cacacagacc gcctgccgtg tttcagaccc acttaagcgc ctgttcaagt tgggtaagcc | 7140 |
| gctaacagct gaagacaagc aggacgaaga caggcgacga gcactgagtg acgaggttag | 7200 |
| caagtggttc cggacaggct tgggggccga actggaggtg gcactaacat ctaggtatga | 7260 |
| ggtagagggc tgcaaaagta tcctcatagc catggccacc ttggcgaggg acattaaggc | 7320 |
| gtttaagaaa ttgagaggac ctgttataca cctctacggc ggtcctagat tggtgcgtta | 7380 |
| atacacagaa ttctgattgg atcccaaacg ggccctctag actcgagcgg ccgccactgt | 7440 |
| gctggatatc tgcagaattc caccacactg gactagtgga tctatggcgt acccatacga | 7500 |
| tgttccagat tacgctagct tgagatctac catgtctcag agcaaccggg agctggtggt | 7560 |
| tgactttctc tcctacaagc tttcccagaa aggatacagc tggagtcagt ttagtgatgt | 7620 |
| ggaagagaac aggactgagg ccccagaagg gactgaatcg gagatggaga cccccagtgc | 7680 |
| catcaatggc aacccatcct ggcacctggc agacagcccc gcggtgaatg gagccactgc | 7740 |
| gcacagcagc agtttggatg cccgggaggt gatccccatg gcagcagtaa agcaagcgct | 7800 |
| gagggaggca ggcgacgagt ttgaactgcg gtaccggcgg gcattcagtg acctgacatc | 7860 |
| ccagctccac atcaccccag ggacagcata tcagagcttt gaacaggtag tgaatgaact | 7920 |
| cttccgggat ggggtagcca ttcttcgcat tgtggccttt ttctccttcg gcggggcact | 7980 |
| gtgcgtggaa agcgtagaca aggagatgca ggtattggtg agtcggatcg cagcttggat | 8040 |
| ggccacttac ctgaatgacc acctagagcc ttggatccag gagaacggcg gctgggatac | 8100 |
| ttttgtggaa ctctatggga acaatgcagc agccgagagc cgaaagggcc aggaacgctt | 8160 |
| caaccgctgg ttcctgacgg gcatgactgt ggccggcgtg gttctgctgg gctcactctt | 8220 |
| cagtcggaaa tgaagatccg agctcggtac caagcttaag tttgggtaat taattgaatt | 8280 |
| acatccctac gcaaacgttt tacggccgcc ggtggcgccc gcgcccggcg gcccgtcctt | 8340 |
| ggccgttgca ggccactccg gtggctcccg tcgtccccga cttccaggcc cagcagatgc | 8400 |
| agcaactcat cagcgccgta aatgcgctga caatgagaca gaacgcaatt gctcctgcta | 8460 |
| ggcctcccaa accaaagaag aagaagacaa ccaaaccaaa gccgaaaacg cagcccaaga | 8520 |
| agatcaacgg aaaaacgcag cagcaaaaga agaaagacaa gcaagccgac aagaagaaga | 8580 |
| agaaacccgg aaaaagagaa agaatgtgca tgaagattga aaatgactgt atcttcgtat | 8640 |
| gcggctagcc acagtaacgt agtgtttcca gacatgtcgg gcaccgcact atcatgggtg | 8700 |
| cagaaaatct cgggtggtct gggggccttc gcaatcggcg ctatcctggt gctggttgtg | 8760 |
| gtcacttgca ttgggctccg cagataagtt agggtaggca atggcattga tatagcaaga | 8820 |
| aaattgaaaa cagaaaaagt tagggtaagc aatggcatat aaccataact gtataacttg | 8880 |
| taacaaagcg caacaagacc tgcgcaattg gccccgtggt ccgcctcacg gaaactcggg | 8940 |
| gcaactcata ttgacacatt aattggcaat aattggaagc ttacataagc ttaattcgac | 9000 |
| gaataattgg atttttattt tattttgcaa ttggttttta atatttccaa aaaaaaaaaa | 9060 |
| aaaaaaaaaa aaaaaaaaaa aaaaaaaaaa aaaaaaaaaa aaaaaaaaaa aaaaaaaact | 9120 |
| agtgatcata atcagccata ccacatttgt agaggtttta cttgctttaa aaaacctccc | 9180 |
| acacctcccc ctgaacctga aacataaaat gaatgcaatt gttgttgtta acttgtttat | 9240 |
| tgcagcttat aatggttaca aataaagcaa tagcatcaca aatttcacaa ataaagcatt | 9300 |
| tttttcactg cattctagtt gtggtttgtc caaactcatc aatgtatctt atcatgtctg | 9360 |
| gatctagtct gcattaatga atcggccaac gcgcggggag aggcggtttg cgtattgggc | 9420 |
| gctcttccgc ttcctcgctc actgactcgc tgcgctcggt cgttcggctg cggcgagcgg | 9480 |
| tatcagctca ctcaaaggcg gtaatacggt tatccacaga atcaggggat aacgcaggaa | 9540 |
| agaacatgtg agcaaaaggc cagcaaaagg ccaggaaccg taaaaaggcc gcgttgctgg | 9600 |
| cgtttttcca taggctccgc ccccctgacg agcatcacaa aaatcgacgc tcaagtcaga | 9660 |
| ggtggcgaaa cccgacagga ctataaagat accaggcgtt tccccctgga agctccctcg | 9720 |
| tgcgctctcc tgttccgacc ctgccgctta ccggatacct gtccgccttt ctcccttcgg | 9780 |
| gaagcgtggc gctttctcaa tgctcgcgct gtaggtatct cagttcggtg taggtcgttc | 9840 |
| gctccaagct gggctgtgtg cacgaacccc ccgttcagcc cgaccgctgc gccttatccg | 9900 |
| gtaactatcg tcttgagtcc aacccggtaa gacacgactt atcgccactg gcagcagcca | 9960 |
| ctggtaacag gattagcaga gcgaggtatg taggcggtgc tacagagttc ttgaagtggt | 10020 |
| ggcctaacta cggctacact agaaggacag tatttggtat ctgcgctctg ctgaagccag | 10080 |
| ttaccttcgg aaaaagagtt ggtagctctt gatccggcaa acaaaccacc gctggtagcg | 10140 |
| gtggtttttt tgtttgcaag cagcagatta cgcgcagaaa aaaaggatct caagaagatc | 10200 |
| ctttgatctt ttctacgggg cattctgacg ctcagtggaa cgaaaactca cgttaaggga | 10260 |
| ttttggtcat gagattatca aaaaggatct tcacctagat ccttttaaat taaaaatgaa | 10320 |
| gttttaaatc aatctaaagt atatatgagt aaacttggtc tgacagttac caatgcttaa | 10380 |
| tcagtgaggc acctatctca gcgatctgtc tatttcgttc atccatagtt gcctgactcc | 10440 |
| ccgtcgtgta gataactacg atacgggagg gcttaccatc tggccccagt gctgcaatga | 10500 |
| taccgcgaga cccacgctca ccggctccag atttatcagc aataaaccag ccagccggaa | 10560 |
| gggccgagcg cagaagtggt cctgcaactt tatccgcctc catccagtct attaattgtt | 10620 |
| gccgggaagc tagagtaagt agttcgccag ttaatagttt gcgcaacgtt gttgccattg | 10680 |
| ctacaggcat cgtggtgtca cgctcgtcgt ttggtatggc ttcattcagc tccggttccc | 10740 |
| aacgatcaag gcgagttaca tgatccccca tgttgtgcaa aaaagcggtt agctccttcg | 10800 |
| gtcctccgat cgttgtcaga agtaagttgg ccgcagtgtt atcactcatg gttatggcag | 10860 |
| cactgcataa ttctcttact gtcatgccat ccgtaagatg cttttctgtg actggtgagt | 10920 |
| actcaaccaa gtcattctga gaatagtgta tgcggcgacc gagttgctct tgcccggcgt | 10980 |
| caatacggga taataccgcg ccacatagca gaactttaaa agtgctcatc attggaaaac | 11040 |
| gttcttcggg gcgaaaactc tcaaggatct taccgctgtt gagatccagt tcgatgtaac | 11100 |
| ccactcgtgc acccaactga tcttcagcat cttttacttt caccagcgtt tctgggtgag | 11160 |
| caaaaacagg aaggcaaaat gccgcaaaaa agggaataag ggcgacacgg aaatgttgaa | 11220 |
| tactcatact cttccttttt caatattatt gaagcattta tcagggttat tgtctcatga | 11280 |
| gcggatacat atttgaatgt atttagaaaa ataaacaaat aggggttccg cgcacatttc | 11340 |
| cccgaaaagt gccacctgac gtctaagaaa ccattattat catgacatta acctataaaa | 11400 |
| ataggcgtat cacgaggccc tttcgtctcg cgcgtttcgg tgatgacggt gaaaacctct | 11460 |
| gacacatgca gctcccggag acggtcacag cttctgtcta agcggatgcc gggagcagac | 11520 |
| aagcccgtca gggcgcgtca gcgggtgttg gcgggtgtcg gggctggctt aactatgcgg | 11580 |
| catcagagca gattgtactg agagtgcacc atatcgacgc tctcccttat gcgactcctg | 11640 |
| cattaggaag cagcccagta ctaggttgag gccgttgagc accgccgccg caaggaatgg | 11700 |
| tgcatgcgta atcaattacg gggtcattag ttcatagccc atatatggag ttccgcgtta | 11760 |
| cataacttac ggtaaatggc ccgcctggct gaccgcccaa cgacccccgc ccattgacgt | 11820 |
| caataatgac gtatgttccc atagtaacgc caatagggac tttccattga cgtcaatggg | 11880 |
| tggagtattt acggtaaact gcccacttgg cagtacatca agtgtatcat atgccaagta | 11940 |
| cgccccctat tgacgtcaat gacggtaaat ggcccgcctg gcattatgcc cagtacatga | 12000 |
| ccttatggga ctttcctact tggcagtaca tctacgtatt agtcatcgct attaccatgg | 12060 |
| tgatgcggtt ttggcagtac atcaatgggc gtggatagcg gtttgactca cggggatttc | 12120 |
| caagtctcca ccccattgac gtcaatggga gtttgttttg gcaccaaaat caacgggact | 12180 |
| ttccaaaatg tcgtaacaac tccgccccat tgacgcaaat gggcggtagg cgtgtacggt | 12240 |
| gggaggtcta tataagcaga gctctctggc taactagaga acccactgct taactggctt | 12300 |
| atcgaaatta atacgactca ctatagggag accggaagct tgaattc | 12347 |
| SEQ ID NO: 68 | |
| atggcggatg tgtgacatac acgacgccaa aagattttgt tccagctcct gccacctccg | 60 |
| ctacgcgaga gattaaccac ccacgatggc cgccaaagtg catgttgata ttgaggctga | 120 |
| cagcccattc atcaagtctt tgcagaaggc atttccgtcg ttcgaggtgg agtcattgca | 180 |
| ggtcacacca aatgaccatg caaatgccag agcattttcg cacctggcta ccaaattgat | 240 |
| cgagcaggag actgacaaag acacactcat cttggatatc ggcagtgcgc cttccaggag | 300 |
| aatgatgtct acgcacaaat accactgcgt atgccctatg cgcagcgcag aagaccccga | 360 |
| aaggctcgat agctacgcaa agaaactggc agcggcctcc gggaaggtgc tggatagaga | 420 |
| gatcgcagga aaaatcaccg acctgcagac cgtcatggct acgccagacg ctgaatctcc | 480 |
| taccttttgc ctgcatacag acgtcacgtg tcgtacggca gccgaagtgg ccgtatacca | 540 |
| ggacgtgtat gctgtacatg caccaacatc gctgtaccat caggcgatga aaggtgtcag | 600 |
| aacggcgtat tggattgggt ttgacaccac cccgtttatg tttgacgcgc tagcaggcgc | 660 |
| gtatccaacc tacgccacaa actgggccga cgagcaggtg ttacaggcca ggaacatagg | 720 |
| actgtgtgca gcatccttga ctgagggaag actcggcaaa ctgtccattc tccgcaagaa | 780 |
| gcaattgaaa ccttgcgaca cagtcatgtt ctcggtagga tctacattgt acactgagag | 840 |
| cagaaagcta ctgaggagct ggcacttacc ctccgtattc cacctgaaag gtaaacaatc | 900 |
| ctttacctgt aggtgcgata ccatcgtatc atgtgaaggg tacgtagtta agaaaatcac | 960 |
| tatgtgcccc ggcctgtacg gtaaaacggt agggtacgcc gtgacgtatc acgcggaggg | 1020 |
| attcctagtg tgcaagacca cagacactgt caaaggagaa agagtctcat tccctgtatg | 1080 |
| cacctacgtc ccctcaacca tctgtgatca aatgactggc atactagcga ccgacgtcac | 1140 |
| accggaggac gcacagaagt tgttagtggg attgaatcag aggatagttg tgaacggaag | 1200 |
| aacacagcga aacactaaca cgatgaagaa ctatctgctt ccgattgtgg ccgtcgcatt | 1260 |
| tagcaagtgg gcgagggaat acaaggcaga ccttgatgat gaaaaacctc tgggtgtccg | 1320 |
| agagaggtca cttacttgct gctgcttgtg ggcatttaaa acgaggaaga tgcacaccat | 1380 |
| gtacaagaaa ccagacaccc agacaatagt gaaggtgcct tcagagttta actcgttcgt | 1440 |
| catcccgagc ctatggtcta caggcctcgc aatcccagtc agatcacgca ttaagatgct | 1500 |
| tttggccaag aagaccaagc gagagttaat acctgttctc gacgcgtcgt cagccaggga | 1560 |
| tgctgaacaa gaggagaagg agaggttgga ggccgagctg actagagaag ccttaccacc | 1620 |
| cctcgtcccc atcgcgccgg cggagacggg agtcgtcgac gtcgacgttg aagaactaga | 1680 |
| gtatcacgca ggtgcagggg tcgtggaaac acctcgcagc gcgttgaaag tcaccgcaca | 1740 |
| gccgaacgac gtactactag gaaattacgt agttctgtcc ccgcagaccg tgctcaagag | 1800 |
| ctccaagttg gcccccgtgc accctctagc agagcaggtg aaaataataa cacataacgg | 1860 |
| gagggccggc ggttaccagg tcgacggata tgacggcagg gtcctactac catgtggatc | 1920 |
| ggccattccg gtccctgagt ttcaagcttt gagcgagagc gccactatgg tgtacaacga | 1980 |
| aagggagttc gtcaacagga aactatacca tattgccgtt cacggaccgt cgctgaacac | 2040 |
| cgacgaggag aactacgaga aagtcagagc tgaaagaact gacgccgagt acgtgttcga | 2100 |
| cgtagataaa aaatgctgcg tcaagagaga ggaagcgtcg ggtttggtgt tggtgggaga | 2160 |
| gctaaccaac cccccgttcc atgaattcgc ctacgaaggg ctgaagatca ggccgtcggc | 2220 |
| accatataag actacagtag taggagtctt tggggttccg ggatcaggca agtctgctat | 2280 |
| tattaagagc ctcgtgacca aacacgatct ggtcaccagc ggcaagaagg agaactgcca | 2340 |
| ggaaatagtt aacgacgtga agaagcaccg cgggaagggg acaagtaggg aaaacagtga | 2400 |
| ctccatcctg ctaaacgggt gtcgtcgtgc cgtggacatc ctatatgtgg acgaggcttt | 2460 |
| cgctagccat tccggtactc tgctggccct aattgctctt gttaaacctc ggagcaaagt | 2520 |
| ggtgttatgc ggagacccca agcaatgcgg attcttcaat atgatgcagc ttaaggtgaa | 2580 |
| cttcaaccac aacatctgca ctgaagtatg tcataaaagt atatccagac gttgcacgcg | 2640 |
| tccagtcacg gccatcgtgt ctacgttgca ctacggaggc aagatgcgca cgaccaaccc | 2700 |
| gtgcaacaaa cccataatca tagacaccac aggacagacc aagcccaagc caggagacat | 2760 |
| cgtgttaaca tgcttccgag gctgggcaaa gcagctgcag ttggactacc gtggacacga | 2820 |
| agtcatgaca gcagcagcat ctcagggcct cacccgcaaa ggggtatacg ccgtaaggca | 2880 |
| gaaggtgaat gaaaatccct tgtatgcccc tgcgtcggag cacgtgaatg tactgctgac | 2940 |
| gcgcactgag gataggctgg tgtggaaaac gctggccggc gatccctgga ttaaggtcct | 3000 |
| atcaaacatt ccacagggta actttacggc cacattggaa gaatggcaag aagaacacga | 3060 |
| caaaataatg aaggtgattg aaggaccggc tgcgcctgtg gacgcgttcc agaacaaagc | 3120 |
| gaacgtgtgt tgggcgaaaa gcctggtgcc tgtcctggac actgccggaa tcagattgac | 3180 |
| agcagaggag tggagcacca taattacagc atttaaggag gacagagctt actctccagt | 3240 |
| ggtggccttg aatgaaattt gcaccaagta ctatggagtt gacctggaca gtggcctgtt | 3300 |
| ttctgccccg aaggtgtccc tgtattacga gaacaaccac tgggataaca gacctggtgg | 3360 |
| aaggatgtat ggattcaatg ccgcaacagc tgccaggctg gaagctagac ataccttcct | 3420 |
| gaaggggcag tggcatacgg gcaagcaggc agttatcgca gaaagaaaaa tccaaccgct | 3480 |
| ttctgtgctg gacaatgtaa ttcctatcaa ccgcaggctg ccgcacgccc tggtggctga | 3540 |
| gtacaagacg gttaaaggca gtagggttga gtggctggtc aataaagtaa gagggtacca | 3600 |
| cgtcctgctg gtgagtgagt acaacctggc tttgcctcga cgcagggtca cttggttgtc | 3660 |
| accgctgaat gtcacaggcg ccgataggtg ctacgaccta agtttaggac tgccggctga | 3720 |
| cgccggcagg ttcgacttgg tctttgtgaa cattcacacg gaattcagaa tccaccacta | 3780 |
| ccagcagtgt gtcgaccacg ccatgaagct gcagatgctt gggggagatg cgctacgact | 3840 |
| gctaaaaccc ggcggcatct tgatgagagc ttacggatac gccgataaaa tcagcgaagc | 3900 |
| cgttgtttcc tccttaagca gaaagttctc gtctgcaaga gtgttgcgcc cggattgtgt | 3960 |
| caccagcaat acagaagtgt tcttgctgtt ctccaacttt gacaacggaa agagaccctc | 4020 |
| tacgctacac cagatgaata ccaagctgag tgccgtgtat gccggagaag ccatgcacac | 4080 |
| ggccgggtgt gcaccatcct acagagttaa gagagcagac atagccacgt gcacagaagc | 4140 |
| ggctgtggtt aacgcagcta acgcccgtgg aactgtaggg gatggcgtat gcagggccgt | 4200 |
| ggcgaagaaa tggccgtcag cctttaaggg agcagcaaca ccagtgggca caattaaaac | 4260 |
| agtcatgtgc ggctcgtacc ccgtcatcca cgctgtagcg cctaatttct ctgccacgac | 4320 |
| tgaagcggaa ggggaccgcg aattggccgc tgtctaccgg gcagtggccg ccgaagtaaa | 4380 |
| cagactgtca ctgagcagcg tagccatccc gctgctgtcc acaggagtgt tcagcggcgg | 4440 |
| aagagatagg ctgcagcaat ccctcaacca tctattcaca gcaatggacg ccacggacgc | 4500 |
| tgacgtgacc atctactgca gagacaaaag ttgggagaag aaaatccagg aagccattga | 4560 |
| catgaggacg gctgtggagt tgctcaatga tgacgtggag ctgaccacag acttggtgag | 4620 |
| agtgcacccg gacagcagcc tggtgggtcg taagggctac agtaccactg acgggtcgct | 4680 |
| gtactcgtac tttgaaggta cgaaattcaa ccaggctgct attgatatgg cagagatact | 4740 |
| gacgttgtgg cccagactgc aagaggcaaa cgaacagata tgcctatacg cgctgggcga | 4800 |
| aacaatggac aacatcagat ccaaatgtcc ggtgaacgat tccgattcat caacacctcc | 4860 |
| caggacagtg ccctgcctgt gccgctacgc aatgacagca gaacggatcg cccgccttag | 4920 |
| gtcacaccaa gttaaaagca tggtggtttg ctcatctttt cccctcccga aataccatgt | 4980 |
| agatggggtg cagaaggtaa agtgcgagaa ggttctcctg ttcgacccga cggtaccttc | 5040 |
| agtggttagt ccgcggaagt atgccgcatc tacgacggac cactcagatc ggtcgttacg | 5100 |
| agggtttgac ttggactgga ccaccgactc gtcttccact gccagcgata ccatgtcgct | 5160 |
| acccagtttg cagtcgtgtg acatcgactc gatctacgag ccaatggctc ccatagtagt | 5220 |
| gacggctgac gtacaccctg aacccgcagg catcgcggac ctggcggcag atgtgcaccc | 5280 |
| tgaacccgca gaccatgtgg acctcgagaa cccgattcct ccaccgcgcc cgaagagagc | 5340 |
| tgcatacctt gcctcccgcg cggcggagcg accggtgccg gcgccgagaa agccgacgcc | 5400 |
| tgccccaagg actgcgttta ggaacaagct gcctttgacg ttcggcgact ttgacgagca | 5460 |
| cgaggtcgat gcgttggcct ccgggattac tttcggagac ttcgacgacg tcctgcgact | 5520 |
| aggccgcgcg ggtgcatata ttttctcctc ggacactggc agcggacatt tacaacaaaa | 5580 |
| atccgttagg cagcacaatc tccagtgcgc acaactggat gcggtccagg aggagaaaat | 5640 |
| gtacccgcca aaattggata ctgagaggga gaagctgttg ctgctgaaaa tgcagatgca | 5700 |
| cccatcggag gctaataaga gtcgatacca gtctcgcaaa gtggagaaca tgaaagccac | 5760 |
| ggtggtggac aggctcacat cgggggccag attgtacacg ggagcggacg taggccgcat | 5820 |
| accaacatac gcggttcggt acccccgccc cgtgtactcc cctaccgtga tcgaaagatt | 5880 |
| ctcaagcccc gatgtagcaa tcgcagcgtg caacgaatac ctatccagaa attacccaac | 5940 |
| agtggcgtcg taccagataa cagatgaata cgacgcatac ttggacatgg ttgacgggtc | 6000 |
| ggatagttgc ttggacagag cgacattctg cccggcgaag ctccggtgct acccgaaaca | 6060 |
| tcatgcgtac caccagccga ctgtacgcag tgccgtcccg tcaccctttc agaacacact | 6120 |
| acagaacgtg ctagcggccg ccaccaagag aaactgcaac gtcacgcaaa tgcgagaact | 6180 |
| acccaccatg gactcggcag tgttcaacgt ggagtgcttc aagcgctatg cctgctccgg | 6240 |
| agaatattgg gaagaatatg ctaaacaacc tatccggata accactgaga acatcactac | 6300 |
| ctatgtgacc aaattgaaag gcccgaaagc tgctgccttg ttcgctaaga cccacaactt | 6360 |
| ggttccgctg caggaggttc ccatggacag attcacggtc gacatgaaac gagatgtcaa | 6420 |
| agtcactcca gggacgaaac acacagagga aagacccaaa gtccaggtaa ttcaagcagc | 6480 |
| ggagccattg gcgaccgctt acctgtgcgg catccacagg gaattagtaa ggagactaaa | 6540 |
| tgctgtgtta cgccctaacg tgcacacatt gtttgatatg tcggccgaag actttgacgc | 6600 |
| gatcatcgcc tctcacttcc acccaggaga cccggttcta gagacggaca ttgcatcatt | 6660 |
| cgacaaaagc caggacgact ccttggctct tacaggttta atgatcctcg aagatctagg | 6720 |
| ggtggatcag tacctgctgg acttgatcga ggcagccttt ggggaaatat ccagctgtca | 6780 |
| cctaccaact ggcacgcgct tcaagttcgg agctatgatg aaatcgggca tgtttctgac | 6840 |
| tttgtttatt aacactgttt tgaacatcac catagcaagc agggtactgg agcagagact | 6900 |
| cactgactcc gcctgtgcgg ccttcatcgg cgacgacaac atcgttcacg gagtgatctc | 6960 |
| cgacaagctg atggcggaga ggtgcgcgtc gtgggtcaac atggaggtga agatcattga | 7020 |
| cgctgtcatg ggcgaaaaac ccccatattt ttgtggggga ttcatagttt ttgacagcgt | 7080 |
| cacacagacc gcctgccgtg tttcagaccc acttaagcgc ctgttcaagt tgggtaagcc | 7140 |
| gctaacagct gaagacaagc aggacgaaga caggcgacga gcactgagtg acgaggttag | 7200 |
| caagtggttc cggacaggct tgggggccga actggaggtg gcactaacat ctaggtatga | 7260 |
| ggtagagggc tgcaaaagta tcctcatagc catggccacc ttggcgaggg acattaaggc | 7320 |
| gtttaagaaa ttgagaggac ctgttataca cctctacggc ggtcctagat tggtgcgtta | 7380 |
| atacacagaa ttctgattgg atcccaaacg ggccctctag actcgagcgg ccgccactgt | 7440 |
| gctggatatc tgcagaattc atgcatggag atacacctac attgcatgaa tatatgttag | 7500 |
| atttgcaacc agagacaact gatctctact gttatgagca attaaatgac agctcagagg | 7560 |
| aggaggatga aatagatggt ccagctggac aagcagaacc ggacagagcc cattacaata | 7620 |
| ttgtaacctt ttgttgcaag tgtgactcta cgcttcggtt gtgcgtacaa agcacacacg | 7680 |
| tagacattcg tactttggaa gacctgttaa tgggcacact aggaattgtg tgccccatct | 7740 |
| gttctcagaa accaggatct atggcgtacc catacgatgt tccagattac gctagcttga | 7800 |
| gatctaccat gtctcagagc aaccgggagc tggtggttga ctttctctcc tacaagcttt | 7860 |
| cccagaaagg atacagctgg agtcagttta gtgatgtgga agagaacagg actgaggccc | 7920 |
| cagaagggac tgaatcggag atggagaccc ccagtgccat caatggcaac ccatcctggc | 7980 |
| acctggcaga cagccccgcg gtgaatggag ccactgcgca cagcagcagt ttggatgccc | 8040 |
| gggaggtgat ccccatggca gcagtaaagc aagcgctgag ggaggcaggc gacgagtttg | 8100 |
| aactgcggta ccggcgggca ttcagtgacc tgacatccca gctccacatc accccaggga | 8160 |
| cagcatatca gagctttgaa caggtagtga atgaactctt ccgggatggg gtagccattc | 8220 |
| ttcgcattgt ggcctttttc tccttcggcg gggcactgtg cgtggaaagc gtagacaagg | 8280 |
| agatgcaggt attggtgagt cggatcgcag cttggatggc cacttacctg aatgaccacc | 8340 |
| tagagccttg gatccaggag aacggcggct gggatacttt tgtggaactc tatgggaaca | 8400 |
| atgcagcagc cgagagccga aagggccagg aacgcttcaa ccgctggttc ctgacgggca | 8460 |
| tgactgtggc cggcgtggtt ctgctgggct cactcttcag tcggaaatga agatccaagc | 8520 |
| ttaagtttgg gtaattaatt gaattacatc cctacgcaaa cgttttacgg ccgccggtgg | 8580 |
| cgcccgcgcc cggcggcccg tccttggccg ttgcaggcca ctccggtggc tcccgtcgtc | 8640 |
| cccgacttcc aggcccagca gatgcagcaa ctcatcagcg ccgtaaatgc gctgacaatg | 8700 |
| agacagaacg caattgctcc tgctaggcct cccaaaccaa agaagaagaa gacaaccaaa | 8760 |
| ccaaagccga aaacgcagcc caagaagatc aacggaaaaa cgcagcagca aaagaagaaa | 8820 |
| gacaagcaag ccgacaagaa gaagaagaaa cccggaaaaa gagaaagaat gtgcatgaag | 8880 |
| attgaaaatg actgtatctt cgtatgcggc tagccacagt aacgtagtgt ttccagacat | 8940 |
| gtcgggcacc gcactatcat gggtgcagaa aatctcgggt ggtctggggg ccttcgcaat | 9000 |
| cggcgctatc ctggtgctgg ttgtggtcac ttgcattggg ctccgcagat aagttagggt | 9060 |
| aggcaatggc attgatatag caagaaaatt gaaaacagaa aaagttaggg taagcaatgg | 9120 |
| catataacca taactgtata acttgtaaca aagcgcaaca agacctgcgc aattggcccc | 9180 |
| gtggtccgcc tcacggaaac tcggggcaac tcatattgac acattaattg gcaataattg | 9240 |
| gaagcttaca taagcttaat tcgacgaata attggatttt tattttattt tgcaattggt | 9300 |
| ttttaatatt tccaaaaaaa aaaaaaaaaa aaaaaaaaaa aaaaaaaaaa aaaaaaaaaa | 9360 |
| aaaaaaaaaa aaaaaaaaaa aaactagtga tcataatcag ccataccaca tttgtagagg | 9420 |
| ttttacttgc tttaaaaaac ctcccacacc tccccctgaa cctgaaacat aaaatgaatg | 9480 |
| caattgttgt tgttaacttg tttattgcag cttataatgg ttacaaataa agcaatagca | 9540 |
| tcacaaattt cacaaataaa gcattttttt cactgcattc tagttgtggt ttgtccaaac | 9600 |
| tcatcaatgt atcttatcat gtctggatct agtctgcatt aatgaatcgg ccaacgcgcg | 9660 |
| gggagaggcg gtttgcgtat tgggcgctct tccgcttcct cgctcactga ctcgctgcgc | 9720 |
| tcggtcgttc ggctgcggcg agcggtatca gctcactcaa aggcggtaat acggttatcc | 9780 |
| acagaatcag gggataacgc aggaaagaac atgtgagcaa aaggccagca aaaggccagg | 9840 |
| aaccgtaaaa aggccgcgtt gctggcgttt ttccataggc tccgcccccc tgacgagcat | 9900 |
| cacaaaaatc gacgctcaag tcagaggtgg cgaaacccga caggactata aagataccag | 9960 |
| gcgtttcccc ctggaagctc cctcgtgcgc tctcctgttc cgaccctgcc gcttaccgga | 10020 |
| tacctgtccg cctttctccc ttcgggaagc gtggcgcttt ctcaatgctc gcgctgtagg | 10080 |
| tatctcagtt cggtgtaggt cgttcgctcc aagctgggct gtgtgcacga accccccgtt | 10140 |
| cagcccgacc gctgcgcctt atccggtaac tatcgtcttg agtccaaccc ggtaagacac | 10200 |
| gacttatcgc cactggcagc agccactggt aacaggatta gcagagcgag gtatgtaggc | 10260 |
| ggtgctacag agttcttgaa gtggtggcct aactacggct acactagaag gacagtattt | 10320 |
| ggtatctgcg ctctgctgaa gccagttacc ttcggaaaaa gagttggtag ctcttgatcc | 10380 |
| ggcaaacaaa ccaccgctgg tagcggtggt ttttttgttt gcaagcagca gattacgcgc | 10440 |
| agaaaaaaag gatctcaaga agatcctttg atcttttcta cggggcattc tgacgctcag | 10500 |
| tggaacgaaa actcacgtta agggattttg gtcatgagat tatcaaaaag gatcttcacc | 10560 |
| tagatccttt taaattaaaa atgaagtttt aaatcaatct aaagtatata tgagtaaact | 10620 |
| tggtctgaca gttaccaatg cttaatcagt gaggcaccta tctcagcgat ctgtctattt | 10680 |
| cgttcatcca tagttgcctg actccccgtc gtgtagataa ctacgatacg ggagggctta | 10740 |
| ccatctggcc ccagtgctgc aatgataccg cgagacccac gctcaccggc tccagattta | 10800 |
| tcagcaataa accagccagc cggaagggcc gagcgcagaa gtggtcctgc aactttatcc | 10860 |
| gcctccatcc agtctattaa ttgttgccgg gaagctagag taagtagttc gccagttaat | 10920 |
| agtttgcgca acgttgttgc cattgctaca ggcatcgtgg tgtcacgctc gtcgtttggt | 10980 |
| atggcttcat tcagctccgg ttcccaacga tcaaggcgag ttacatgatc ccccatgttg | 11040 |
| tgcaaaaaag cggttagctc cttcggtcct ccgatcgttg tcagaagtaa gttggccgca | 11100 |
| gtgttatcac tcatggttat ggcagcactg cataattctc ttactgtcat gccatccgta | 11160 |
| agatgctttt ctgtgactgg tgagtactca accaagtcat tctgagaata gtgtatgcgg | 11220 |
| cgaccgagtt gctcttgccc ggcgtcaata cgggataata ccgcgccaca tagcagaact | 11280 |
| ttaaaagtgc tcatcattgg aaaacgttct tcggggcgaa aactctcaag gatcttaccg | 11340 |
| ctgttgagat ccagttcgat gtaacccact cgtgcaccca actgatcttc agcatctttt | 11400 |
| actttcacca gcgtttctgg gtgagcaaaa acaggaaggc aaaatgccgc aaaaaaggga | 11460 |
| ataagggcga cacggaaatg ttgaatactc atactcttcc tttttcaata ttattgaagc | 11520 |
| atttatcagg gttattgtct catgagcgga tacatatttg aatgtattta gaaaaataaa | 11580 |
| caaatagggg ttccgcgcac atttccccga aaagtgccac ctgacgtcta agaaaccatt | 11640 |
| attatcatga cattaaccta taaaaatagg cgtatcacga ggccctttcg tctcgcgcgt | 11700 |
| ttcggtgatg acggtgaaaa cctctgacac atgcagctcc cggagacggt cacagcttct | 11760 |
| gtctaagcgg atgccgggag cagacaagcc cgtcagggcg cgtcagcggg tgttggcggg | 11820 |
| tgtcggggct ggcttaacta tgcggcatca gagcagattg tactgagagt gcaccatatc | 11880 |
| gacgctctcc cttatgcgac tcctgcatta ggaagcagcc cagtactagg ttgaggccgt | 11940 |
| tgagcaccgc cgccgcaagg aatggtgcat gcgtaatcaa ttacggggtc attagttcat | 12000 |
| agcccatata tggagttccg cgttacataa cttacggtaa atggcccgcc tggctgaccg | 12060 |
| cccaacgacc cccgcccatt gacgtcaata atgacgtatg ttcccatagt aacgccaata | 12120 |
| gggactttcc attgacgtca atgggtggag tatttacggt aaactgccca cttggcagta | 12180 |
| catcaagtgt atcatatgcc aagtacgccc cctattgacg tcaatgacgg taaatggccc | 12240 |
| gcctggcatt atgcccagta catgacctta tgggactttc ctacttggca gtacatctac | 12300 |
| gtattagtca tcgctattac catggtgatg cggttttggc agtacatcaa tgggcgtgga | 12360 |
| tagcggtttg actcacgggg atttccaagt ctccacccca ttgacgtcaa tgggagtttg | 12420 |
| ttttggcacc aaaatcaacg ggactttcca aaatgtcgta acaactccgc cccattgacg | 12480 |
| caaatgggcg gtaggcgtgt acggtgggag gtctatataa gcagagctct ctggctaact | 12540 |
| agagaaccca ctgcttaact ggcttatcga aattaatacg actcactata gggagaccgg | 12600 |
| aagcttgaat tc | 12612 |
| SEQ ID NO: 69 | |
| gtcgacttct gaggcggaaa gaaccagctg tggaatgtgt gtcagttagg gtgtggaaag | 60 |
| tccccaggct ccccagcagg cagaagtatg caaagcatgc atctcaatta gtcagcaacc | 120 |
| aggtgtggaa agtccccagg ctccccagca ggcagaagta tgcaaagcat gcatctcaat | 180 |
| tagtcagcaa ccatagtccc gcccctaact ccgcccatcc cgcccctaac tccgcccagt | 240 |
| tccgcccatt ctccgcccca tggctgacta atttttttta tttatgcaga ggccgaggcc | 300 |
| gcctcggcct ctgagctatt ccagaagtag tgaggaggct tttttggagg cctaggcttt | 360 |
| tgcaaaaagc tggatcgatc ctgagaactt cagggtgagt ttggggaccc ttgattgttc | 420 |
| tttctttttc gctattgtaa aattcatgtt atatggaggg ggcaaagttt tcagggtgtt | 480 |
| gtttagaatg ggaagatgtc ccttgtatca ccatggaccc tcatgataat tttgtttctt | 540 |
| tcactttcta ctctgttgac aaccattgtc tcctcttatt ttcttttcat tttctgtaac | 600 |
| tttttcgtta aactttagct tgcatttgta acgaattttt aaattcactt ttgtttattt | 660 |
| gtcagattgt aagtactttc tctaatcact tttttttcaa ggcaatcagg gtatattata | 720 |
| ttgtacttca gcacagtttt agagaacaat tgttataatt aaatgataag gtagaatatt | 780 |
| tctgcatata aattctggct ggcgtggaaa tattcttatt ggtagaaaca actacatcct | 840 |
| ggtcatcatc ctgcctttct ctttatggtt acaatgatat acactgtttg agatgaggat | 900 |
| aaaatactct gagtccaaac cgggcccctc tgctaaccat gttcatgcct tcttcttttt | 960 |
| cctacagctc ctgggcaacg tgctggttat tgtgctgtct catcattttg gcaaagaatt | 1020 |
| gtaatacgac tcactatagg gcgaattcgg atccagatct atggcgtacc catacgatgt | 1080 |
| tccagattac gctagcttga gatctaccat gtctcagagc aaccgggagc tggtggttga | 1140 |
| ctttctctcc tacaagcttt cccagaaagg atacagctgg agtcagttta gtgatgtgga | 1200 |
| agagaacagg actgaggccc cagaagggac tgaatcggag atggagaccc ccagtgccat | 1260 |
| caatggcaac ccatcctggc acctggcaga cagccccgcg gtgaatggag ccactgcgca | 1320 |
| cagcagcagt ttggatgccc gggaggtgat ccccatggca gcagtaaagc aagcgctgag | 1380 |
| ggaggcaggc gacgagtttg aactgcggta ccggcgggca ttcagtgacc tgacatccca | 1440 |
| gctccacatc accccaggga cagcatatca gagctttgaa caggtagtga atgaactctt | 1500 |
| ccgggatggg gtaaactggg gtcgcattgt ggcctttttc tccttcggcg gggcactgtg | 1560 |
| cgtggaaagc gtagacaagg agatgcaggt attggtgagt cggatcgcag cttggatggc | 1620 |
| cacttacctg aatgaccacc tagagccttg gatccaggag aacggcggct gggatacttt | 1680 |
| tgtggaactc tatgggaaca atgcagcagc cgagagccga aagggccagg aacgcttcaa | 1740 |
| ccgctggttc ctgacgggca tgactgtggc cggcgtggtt ctgctgggct cactcttcag | 1800 |
| tcggaaatga agatcttatt aaagcagaac ttgtttattg cagcttataa tggttacaaa | 1860 |
| taaagcaata gcatcacaaa tttcacaaat aaagcatttt tttcactgca ttctagttgt | 1920 |
| ggtttgtcca aactcatcaa tgtatcttat catgtctggt cgactctaga ctcttccgct | 1980 |
| tcctcgctca ctgactcgct gcgctcggtc gttcggctgc ggcgagcggt atcagctcac | 2040 |
| tcaaaggcgg taatacggtt atccacagaa tcaggggata acgcaggaaa gaacatgtga | 2100 |
| gcaaaggcca gcaaaaggcc aggaaccgta aaaaggccgc gttgctggcg ttttttccat | 2160 |
| aggctccgcc cccctgacga gcatcacaaa aatcgacgct caagtcagag gtggcgaaac | 2220 |
| ccgacaggac tataaagata ccaggcgttt ccccctggaa gctccctcgt gcgctctcct | 2280 |
| gttccgaccc tgccgcttac cggatacctg tccgcctttc tcccttcggg aagcgtggcg | 2340 |
| ctttctcaat gctcacgctg taggtatctc agttcggtgt aggtcgttcg ctccaagctg | 2400 |
| ggctgtgtgc acgaaccccc cgttcagccc gaccgctgcg ccttatccgg taactatcgt | 2460 |
| cttgagtcca acccggtaag acacgactta tcgccactgg cagcagccac tggtaacagg | 2520 |
| attagcagag cgaggtatgt aggcggtgct acagagttct tgaagtggtg gcctaactac | 2580 |
| ggctacacta gaaggacagt atttggtatc tgcgctctgc tgaagccagt taccttcgga | 2640 |
| aaaagagttg gtagctcttg atccggcaaa caaaccaccg ctggtagcgg tggttttttt | 2700 |
| gtttgcaagc agcagattac gcgcagaaaa aaaggatctc aagaagatcc tttgatcttt | 2760 |
| tctacggggt ctgacgctca gtggaacgaa aactcacgtt aagggatttt ggtcatgaga | 2820 |
| ttatcaaaaa ggatcttcac ctagatcctt ttaaattaaa aatgaagttt taaatcaatc | 2880 |
| taaagtatat atgagtaaac ttggtctgac agttaccaat gcttaatcag tgaggcacct | 2940 |
| atctcagcga tctgtctatt tcgttcatcc atagttgcct gactccccgt cgtgtagata | 3000 |
| actacgatac gggagggctt accatctggc cccagtgctg caatgatacc gcgagaccca | 3060 |
| cgctcaccgg ctccagattt atcagcaata aaccagccag ccggaagggc cgagcgcaga | 3120 |
| agtggtcctg caactttatc cgcctccatc cagtctatta attgttgccg ggaagctaga | 3180 |
| gtaagtagtt cgccagttaa tagtttgcgc aacgttgttg ccattgctac aggcatcgtg | 3240 |
| gtgtcacgct cgtcgtttgg tatggcttca ttcagctccg gttcccaacg atcaaggcga | 3300 |
| gttacatgat cccccatgtt gtgcaaaaaa gcggttagct ccttcggtcc tccgatcgtt | 3360 |
| gtcagaagta agttggccgc agtgttatca ctcatggtta tggcagcact gcataattct | 3420 |
| cttactgtca tgccatccgt aagatgcttt tctgtgactg gtgagtactc aaccaagtca | 3480 |
| ttctgagaat agtgtatgcg gcgaccgagt tgctcttgcc cggcgtcaat acgggataat | 3540 |
| accgcgccac atagcagaac tttaaaagtg ctcatcattg gaaaacgttc ttcggggcga | 3600 |
| aaactctcaa ggatcttacc gctgttgaga tccagttcga tgtaacccac tcgtgcaccc | 3660 |
| aactgatctt cagcatcttt tactttcacc agcgtttctg ggtgagcaaa aacaggaagg | 3720 |
| caaaatgccg caaaaaaggg aataagggcg acacggaaat gttgaatact catactcttc | 3780 |
| ttttttcaat attattgaag catttatcag ggttattgtc tcatgagcgg atacatattt | 3840 |
| gaatgtattt agaaaaataa acaaataggg gttccgcgca catttccccg aaaagtgcca | 3900 |
| cctgacgtct aagaaaccat tattatcatg acattaacct ataaaaatag gcgtatcacg | 3960 |
| aggccccttt cgtctcgcgc gtttcggtga tgacggtgaa aacctctgac acatgcagct | 4020 |
| cccggagacg gtcacagctt gtctgtaagc ggatgccggg agcagacaag cccgtcaggg | 4080 |
| cgcgtcagcg ggtgttggcg ggtgtcgggg ctggcttaac tatgcggcat cagagcagat | 4140 |
| tgtactgaga gtgcaccata tgcggtgtga aataccgcac agatgcgtaa ggagaaaata | 4200 |
| ccgcatcagg aaattgtaaa cgttaatatt ttgttaaaat tcgcgttaaa tttttgttaa | 4260 |
| atcagctcat tttttaacca ataggccgaa atcggcaaaa tcccttataa atcaaaagaa | 4320 |
| tagaccgaga tagggttgag tgttgttcca gtttggaaca agagtccact attaaagaac | 4380 |
| gtggactcca acgtcaaagg gcgaaaaacc gtctatcagg gcgatggccc actacgtgaa | 4440 |
| ccatcaccct aatcaagttt tttggggtcg aggtgccgta aagcactaaa tcggaaccct | 4500 |
| aaagggagcc cccgatttag agcttgacgg ggaaagccgg cgaacgtggc gagaaaggaa | 4560 |
| gggaagaaag cgaaaggagc gggcgctagg gcgctggcaa gtgtagcggt cacgctgcgc | 4620 |
| gtaaccacca cacccgccgc gcttaatgcg ccgctacagg gcgcgtcgcg ccattcgcca | 4680 |
| ttcaggctac gcaactgttg ggaagggcga tcggtgcggg cctcttcgct attacgccag | 4740 |
| ctggcgaagg ggggatgtgc tgcaaggcga ttaagttggg taacgccagg gttttcccag | 4800 |
| tcacgacgtt gtaaaacgac ggccagtgaa tt | 4832 |
| SEQ ID NO: 70 | |
| gtcgacttct gaggcggaaa gaaccagctg tggaatgtgt gtcagttagg gtgtggaaag | 60 |
| tccccaggct ccccagcagg cagaagtatg caaagcatgc atctcaatta gtcagcaacc | 120 |
| aggtgtggaa agtccccagg ctccccagca ggcagaagta tgcaaagcat gcatctcaat | 180 |
| tagtcagcaa ccatagtccc gcccctaact ccgcccatcc cgcccctaac tccgcccagt | 240 |
| tccgcccatt ctccgcccca tggctgacta atttttttta tttatgcaga ggccgaggcc | 300 |
| gcctcggcct ctgagctatt ccagaagtag tgaggaggct tttttggagg cctaggcttt | 360 |
| tgcaaaaagc tggatcgatc ctgagaactt cagggtgagt ttggggaccc ttgattgttc | 420 |
| tttctttttc gctattgtaa aattcatgtt atatggaggg ggcaaagttt tcagggtgtt | 480 |
| gtttagaatg ggaagatgtc ccttgtatca ccatggaccc tcatgataat tttgtttctt | 540 |
| tcactttcta ctctgttgac aaccattgtc tcctcttatt ttcttttcat tttctgtaac | 600 |
| tttttcgtta aactttagct tgcatttgta acgaattttt aaattcactt ttgtttattt | 660 |
| gtcagattgt aagtactttc tctaatcact tttttttcaa ggcaatcagg gtatattata | 720 |
| ttgtacttca gcacagtttt agagaacaat tgttataatt aaatgataag gtagaatatt | 780 |
| tctgcatata aattctggct ggcgtggaaa tattcttatt ggtagaaaca actacatcct | 840 |
| ggtcatcatc ctgcctttct ctttatggtt acaatgatat acactgtttg agatgaggat | 900 |
| aaaatactct gagtccaaac cgggcccctc tgctaaccat gttcatgcct tcttcttttt | 960 |
| cctacagctc ctgggcaacg tgctggttat tgtgctgtct catcattttg gcaaagaatt | 1020 |
| gtaatacgac tcactatagg gcgaattcgg atccagatct atggcgtacc catacgatgt | 1080 |
| tccagattac gctagcttga gatctaccat gtctcagagc aaccgggagc tggtggttga | 1140 |
| ctttctctcc tacaagcttt cccagaaagg atacagctgg agtcagttta gtgatgtgga | 1200 |
| agagaacagg actgaggccc cagaagggac tgaatcggag atggagaccc ccagtgccat | 1260 |
| caatggcaac ccatcctggc acctggcaga cagccccgcg gtgaatggag ccactgcgca | 1320 |
| cagcagcagt ttggatgccc gggaggtgat ccccatggca gcagtaaagc aagcgctgag | 1380 |
| ggaggcaggc gacgagtttg aactgcggta ccggcgggca ttcagtgacc tgacatccca | 1440 |
| gctccacatc accccaggga cagcatatca gagctttgaa caggtagtga atgaactctt | 1500 |
| ccgggatggg gtagccattc ttcgcattgt ggcctttttc tccttcggcg gggcactgtg | 1560 |
| cgtggaaagc gtagacaagg agatgcaggt attggtgagt cggatcgcag cttggatggc | 1620 |
| cacttacctg aatgaccacc tagagccttg gatccaggag aacggcggct gggatacttt | 1680 |
| tgtggaactc tatgggaaca atgcagcagc cgagagccga aagggccagg aacgcttcaa | 1740 |
| ccgctggttc ctgacgggca tgactgtggc cggcgtggtt ctgctgggct cactcttcag | 1800 |
| tcggaaatga agatcttatt aaagcagaac ttgtttattg cagcttataa tggttacaaa | 1860 |
| taaagcaata gcatcacaaa tttcacaaat aaagcatttt tttcactgca ttctagttgt | 1920 |
| ggtttgtcca aactcatcaa tgtatcttat catgtctggt cgactctaga ctcttccgct | 1980 |
| tcctcgctca ctgactcgct gcgctcggtc gttcggctgc ggcgagcggt atcagctcac | 2040 |
| tcaaaggcgg taatacggtt atccacagaa tcaggggata acgcaggaaa gaacatgtga | 2100 |
| gcaaaggcca gcaaaaggcc aggaaccgta aaaaggccgc gttgctggcg ttttttccat | 2160 |
| aggctccgcc cccctgacga gcatcacaaa aatcgacgct caagtcagag gtggcgaaac | 2220 |
| ccgacaggac tataaagata ccaggcgttt ccccctggaa gctccctcgt gcgctctcct | 2280 |
| gttccgaccc tgccgcttac cggatacctg tccgcctttc tcccttcggg aagcgtggcg | 2340 |
| ctttctcaat gctcacgctg taggtatctc agttcggtgt aggtcgttcg ctccaagctg | 2400 |
| ggctgtgtgc acgaaccccc cgttcagccc gaccgctgcg ccttatccgg taactatcgt | 2460 |
| cttgagtcca acccggtaag acacgactta tcgccactgg cagcagccac tggtaacagg | 2520 |
| attagcagag cgaggtatgt aggcggtgct acagagttct tgaagtggtg gcctaactac | 2580 |
| ggctacacta gaaggacagt atttggtatc tgcgctctgc tgaagccagt taccttcgga | 2640 |
| aaaagagttg gtagctcttg atccggcaaa caaaccaccg ctggtagcgg tggttttttt | 2700 |
| gtttgcaagc agcagattac gcgcagaaaa aaaggatctc aagaagatcc tttgatcttt | 2760 |
| tctacggggt ctgacgctca gtggaacgaa aactcacgtt aagggatttt ggtcatgaga | 2820 |
| ttatcaaaaa ggatcttcac ctagatcctt ttaaattaaa aatgaagttt taaatcaatc | 2880 |
| taaagtatat atgagtaaac ttggtctgac agttaccaat gcttaatcag tgaggcacct | 2940 |
| atctcagcga tctgtctatt tcgttcatcc atagttgcct gactccccgt cgtgtagata | 3000 |
| actacgatac gggagggctt accatctggc cccagtgctg caatgatacc gcgagaccca | 3060 |
| cgctcaccgg ctccagattt atcagcaata aaccagccag ccggaagggc cgagcgcaga | 3120 |
| agtggtcctg caactttatc cgcctccatc cagtctatta attgttgccg ggaagctaga | 3180 |
| gtaagtagtt cgccagttaa tagtttgcgc aacgttgttg ccattgctac aggcatcgtg | 3240 |
| gtgtcacgct cgtcgtttgg tatggcttca ttcagctccg gttcccaacg atcaaggcga | 3300 |
| gttacatgat cccccatgtt gtgcaaaaaa gcggttagct ccttcggtcc tccgatcgtt | 3360 |
| gtcagaagta agttggccgc agtgttatca ctcatggtta tggcagcact gcataattct | 3420 |
| cttactgtca tgccatccgt aagatgcttt tctgtgactg gtgagtactc aaccaagtca | 3480 |
| ttctgagaat agtgtatgcg gcgaccgagt tgctcttgcc cggcgtcaat acgggataat | 3540 |
| accgcgccac atagcagaac tttaaaagtg ctcatcattg gaaaacgttc ttcggggcga | 3600 |
| aaactctcaa ggatcttacc gctgttgaga tccagttcga tgtaacccac tcgtgcaccc | 3660 |
| aactgatctt cagcatcttt tactttcacc agcgtttctg ggtgagcaaa aacaggaagg | 3720 |
| caaaatgccg caaaaaaggg aataagggcg acacggaaat gttgaatact catactcttc | 3780 |
| ttttttcaat attattgaag catttatcag ggttattgtc tcatgagcgg atacatattt | 3840 |
| gaatgtattt agaaaaataa acaaataggg gttccgcgca catttccccg aaaagtgcca | 3900 |
| cctgacgtct aagaaaccat tattatcatg acattaacct ataaaaatag gcgtatcacg | 3960 |
| aggccccttt cgtctcgcgc gtttcggtga tgacggtgaa aacctctgac acatgcagct | 4020 |
| cccggagacg gtcacagctt gtctgtaagc ggatgccggg agcagacaag cccgtcaggg | 4080 |
| cgcgtcagcg ggtgttggcg ggtgtcgggg ctggcttaac tatgcggcat cagagcagat | 4140 |
| tgtactgaga gtgcaccata tgcggtgtga aataccgcac agatgcgtaa ggagaaaata | 4200 |
| ccgcatcagg aaattgtaaa cgttaatatt ttgttaaaat tcgcgttaaa tttttgttaa | 4260 |
| atcagctcat tttttaacca ataggccgaa atcggcaaaa tcccttataa atcaaaagaa | 4320 |
| tagaccgaga tagggttgag tgttgttcca gtttggaaca agagtccact attaaagaac | 4380 |
| gtggactcca acgtcaaagg gcgaaaaacc gtctatcagg gcgatggccc actacgtgaa | 4440 |
| ccatcaccct aatcaagttt tttggggtcg aggtgccgta aagcactaaa tcggaaccct | 4500 |
| aaagggagcc cccgatttag agcttgacgg ggaaagccgg cgaacgtggc gagaaaggaa | 4560 |
| gggaagaaag cgaaaggagc gggcgctagg gcgctggcaa gtgtagcggt cacgctgcgc | 4620 |
| gtaaccacca cacccgccgc gcttaatgcg ccgctacagg gcgcgtcgcg ccattcgcca | 4680 |
| ttcaggctac gcaactgttg ggaagggcga tcggtgcggg cctcttcgct attacgccag | 4740 |
| ctggcgaagg ggggatgtgc tgcaaggcga ttaagttggg taacgccagg gttttcccag | 4800 |
| tcacgacgtt gtaaaacgac ggccagtgaa tt | 4832 |
| SEQ ID NO: 71 | |
| atgactttta acagttttga aggatctaaa acttgtgtac ctgcagacat caataaggaa | 60 |
| gaagaatttg tagaagagtt taatagatta aaaacttttg ctaattttcc aagtggtagt | 120 |
| cctgtttcag catcaacact ggcacgagca gggtttcttt atactggtga aggagatacc | 180 |
| gtgcggtgct ttagttgtca tgcagctgta gatagatggc aatatggaga ctcagcagtt | 240 |
| ggaagacaca ggaaagtatc cccaaattgc agatttatca acggctttta tcttgaaaat | 300 |
| agtgccacgc agtctacaaa ttctggtatc cagaatggtc agtacaaagt tgaaaactat | 360 |
| ctgggaagca gagatcattt tgccttagac aggccatctg agacacatgc agactatctt | 420 |
| ttgagaactg ggcaggttgt agatatatca gacaccatat acccgaggaa ccctgccatg | 480 |
| tattgtgaag aagctagatt aaagtccttt cagaactggc cagactatgc tcacctaacc | 540 |
| ccaagagagt tagcaagtgc tggactctac tacacaggta ttggtgacca agtgcagtgc | 600 |
| ttttgttgtg gtggaaaact gaaaaattgg gaaccttgtg atcgtgcctg gtcagaacac | 660 |
| aggcgacact ttcctaattg cttctttgtt ttgggccgga atcttaatat tcgaagtgaa | 720 |
| tctgatgctg tgagttctga taggaatttc ccaaattcaa caaatcttcc aagaaatcca | 780 |
| tccatggcag attatgaagc acggatcttt acttttggga catggatata ctcagttaac | 840 |
| aaggagcagc ttgcaagagc tggattttat gctttaggtg aaggtgataa agtaaagtgc | 900 |
| tttcactgtg gaggagggct aactgattgg aagcccagtg aagacccttg ggaacaacat | 960 |
| gctaaatggt atccagggtg caaatatctg ttagaacaga agggacaaga atatataaac | 1020 |
| aatattcatt taactcattc acttgaggag tgtctggtaa gaactactga gaaaacacca | 1080 |
| tcactaacta gaagaattga tgataccatc ttccaaaatc ctatggtaca agaagctata | 1140 |
| cgaatggggt tcagtttcaa ggacattaag aaaataatgg aggaaaaaat tcagatatct | 1200 |
| gggagcaact ataaatcact tgaggttctg gttgcagatc tagtgaatgc tcagaaagac | 1260 |
| agtatgcaag atgagtcaag tcagacttca ttacagaaag agattagtac tgaagagcag | 1320 |
| ctaaggcgcc tgcaagagga gaagctttgc aaaatctgta tggatagaaa tattgctatc | 1380 |
| gtttttgttc cttgtggaca tctagtcact tgtaaacaat gtgctgaagc agttgacaag | 1440 |
| tgtcccatgt gctacacagt cattactttc aagcaaaaaa tttttatgtc ttaatctaa | 1499 |
| SEQ ID NO: 72 | |
| Met Thr Phe Asn Ser Phe Glu Gly Ser Lys Thr Cys Val Pro Ala Asp | |
| 1 5 10 15 | |
| Ile Asn Lys Glu Glu Glu Phe Val Glu Glu Phe Asn Arg Leu Lys Thr | |
| 20 25 30 | |
| Phe Ala Asn Phe Pro Ser Gly Ser Pro Val Ser Ala Ser Thr Leu Ala | |
| 35 40 45 | |
| Arg Ala Gly Phe Leu Tyr Thr Gly Glu Gly Asp Thr Val Arg Cys Phe | |
| 50 55 60 | |
| Ser Cys His Ala Ala Val Asp Arg Trp Gln Tyr Gly Asp Ser Ala Val | |
| 65 70 75 80 | |
| Gly Arg His Arg Lys Val Ser Pro Asn Cys Arg Phe Ile Asn Gly Phe | |
| 85 90 95 | |
| Tyr Leu Glu Asn Ser Ala Thr Gln Ser Thr Asn Ser Gly Ile Gln Asn | |
| 100 105 110 | |
| Gly Gln Tyr Lys Val Glu Asn Tyr Leu Gly Ser Arg Asp His Phe Ala | |
| 115 120 125 | |
| Leu Asp Arg Pro Ser Glu Thr His Ala Asp Tyr Leu Leu Arg Thr Gly | |
| 130 135 140 | |
| Gln Val Val Asp Ile Ser Asp Thr Ile Tyr Pro Arg Asn Pro Ala Met | |
| 145 150 155 160 | |
| Tyr Cys Glu Glu Ala Arg Leu Lys Ser Phe Gln Asn Trp Pro Asp Tyr | |
| 165 170 175 | |
| Ala His Leu Thr Pro Arg Glu Leu Ala Ser Ala Gly Leu Tyr Tyr Thr | |
| 180 185 190 | |
| Gly Ile Gly Asp Gln Val Gln Cys Phe Cys Cys Gly Gly Lys Leu Lys | |
| 195 200 205 | |
| Asn Trp Glu Pro Cys Asp Arg Ala Trp Ser Glu His Arg Arg His Phe | |
| 210 215 220 | |
| Pro Asn Cys Phe Phe Val Leu Gly Arg Asn Leu Asn Ile Arg Ser Glu | |
| 225 230 235 240 | |
| Ser Asp Ala Val Ser Ser Asp Arg Asn Phe Pro Asn Ser Thr Asn Leu | |
| 245 250 255 | |
| Pro Arg Asn Pro Ser Met Ala Asp Tyr Glu Ala Arg Ile Phe Thr Phe | |
| 260 265 270 | |
| Gly Thr Trp Ile Tyr Ser Val Asn Lys Glu Gln Leu Ala Arg Ala Gly | |
| 275 280 285 | |
| Phe Tyr Ala Leu Gly Glu Gly Asp Lys Val Lys Cys Phe His Cys Gly | |
| 290 295 300 | |
| Gly Gly Leu Thr Asp Trp Lys Pro Ser Glu Asp Pro Trp Glu Gln His | |
| 305 310 315 320 | |
| Ala Lys Trp Tyr Pro Gly Cys Lys Tyr Leu Leu Glu Gln Lys Gly Gln | |
| 325 330 335 | |
| Glu Tyr Ile Asn Asn Ile His Leu Thr His Ser Leu Glu Glu Cys Leu | |
| 340 345 350 | |
| Val Arg Thr Thr Glu Lys Thr Pro Ser Leu Thr Arg Arg Ile Asp Asp | |
| 355 360 365 | |
| Thr Ile Phe Gln Asn Pro Met Val Gln Glu Ala Ile Arg Met Gly Phe | |
| 370 375 380 | |
| Ser Phe Lys Asp Ile Lys Lys Ile Met Glu Glu Lys Ile Gln Ile Ser | |
| 385 390 395 400 | |
| Gly Ser Asn Tyr Lys Ser Leu Glu Val Leu Val Ala Asp Leu Val Asn | |
| 405 410 415 | |
| Ala Gln Lys Asp Ser Met Gln Asp Glu Ser Ser Gln Thr Ser Leu Gln | |
| 420 425 430 | |
| Lys Glu Ile Ser Thr Glu Glu Gln Leu Arg Arg Leu Gln Glu Glu Lys | |
| 435 440 445 | |
| Leu Cys Lys Ile Cys Met Asp Arg Asn Ile Ala Ile Val Phe Val Pro | |
| 450 455 460 | |
| Cys Gly His Leu Val Thr Cys Lys Gln Cys Ala Glu Ala Val Asp Lys | |
| 465 470 475 480 | |
| Cys Pro Met Cys Tyr Thr Val Ile Thr Phe Lys Gln Lys Ile Phe Met | |
| 485 490 495 | |
| Ser | |
| SEQ ID NO: 73 | |
| gtcgacttct gaggcggaaa gaaccagctg tggaatgtgt gtcagttagg gtgtggaaag | 60 |
| tccccaggct ccccagcagg cagaagtatg caaagcatgc atctcaatta gtcagcaacc | 120 |
| aggtgtggaa agtccccagg ctccccagca ggcagaagta tgcaaagcat gcatctcaat | 180 |
| tagtcagcaa ccatagtccc gcccctaact ccgcccatcc cgcccctaac tccgcccagt | 240 |
| tccgcccatt ctccgcccca tggctgacta atttttttta tttatgcaga ggccgaggcc | 300 |
| gcctcggcct ctgagctatt ccagaagtag tgaggaggct tttttggagg cctaggcttt | 360 |
| tgcaaaaagc tggatcgatc ctgagaactt cagggtgagt ttggggaccc ttgattgttc | 420 |
| tttctttttc gctattgtaa aattcatgtt atatggaggg ggcaaagttt tcagggtgtt | 480 |
| gtttagaatg ggaagatgtc ccttgtatca ccatggaccc tcatgataat tttgtttctt | 540 |
| tcactttcta ctctgttgac aaccattgtc tcctcttatt ttcttttcat tttctgtaac | 600 |
| tttttcgtta aactttagct tgcatttgta acgaattttt aaattcactt ttgtttattt | 660 |
| gtcagattgt aagtactttc tctaatcact tttttttcaa ggcaatcagg gtatattata | 720 |
| ttgtacttca gcacagtttt agagaacaat tgttataatt aaatgataag gtagaatatt | 780 |
| tctgcatata aattctggct ggcgtggaaa taatcttatt ggtagaaaca actacatcct | 840 |
| ggtcatcatc ctgcctttct ctttatggtt acaatgatat acactgtttg agatgaggat | 900 |
| aaaatactct gagtccaaac cgggcccctc tgctaaccat gttcatgcct tcttcttttt | 960 |
| cctacagctc ctgggcaacg tgctggttat tgtgctgtct catcattttg gcaaagaatt | 1020 |
| gtaatacgac tcactatagg gcgaattcgg atccatgact tttaacagtt ttgaaggatc | 1080 |
| taaaacttgt gtacctgcag acatcaataa ggaagaagaa tttgtagaag agtttaatag | 1140 |
| attaaaaact tttgctaatt ttccaagtgg tagtcctgtt tcagcatcaa cactggcacg | 1200 |
| agcagggttt ctttatactg gtgaaggaga taccgtgcgg tgctttagtt gtcatgcagc | 1260 |
| tgtagataga tggcaatatg gagactcagc agttggaaga cacaggaaag tatccccaaa | 1320 |
| ttgcagattt atcaacggct tttatcttga aaatagtgcc acgcagtcta caaattctgg | 1380 |
| tatccagaat ggtcagtaca aagttgaaaa ctatctggga agcagagatc attttgcctt | 1440 |
| agacaggcca tctgagacac atgcagacta tcttttgaga actgggcagg ttgtagatat | 1500 |
| atcagacacc atatacccga ggaaccctgc catgtattgt gaagaagcta gattaaagtc | 1560 |
| ctttcagaac tggccagact atgctcacct aaccccaaga gagttagcaa gtgctggact | 1620 |
| ctactacaca ggtattggtg accaagtgca gtgcttttgt tgtggtggaa aactgaaaaa | 1680 |
| ttgggaacct tgtgatcgtg cctggtcaga acacaggcga cactttccta attgcttctt | 1740 |
| tgttttgggc cggaatctta atattcgaag tgaatctgat gctgtgagtt ctgataggaa | 1800 |
| tttcccaaat tcaacaaatc ttccaagaaa tccatccatg gcagattatg aagcacggat | 1860 |
| ctttactttt gggacatgga tatactcagt taacaaggag cagcttgcaa gagctggatt | 1920 |
| ttatgcttta ggtgaaggtg ataaagtaaa gtgctttcac tgtggaggag ggctaactga | 1980 |
| ttggaagccc agtgaagacc cttgggaaca acatgctaaa tggtatccag ggtgcaaata | 2040 |
| tctgttagaa cagaagggac aagaatatat aaacaatatt catttaactc attcacttga | 2100 |
| ggagtgtctg gtaagaacta ctgagaaaac accatcacta actagaagaa ttgatgatac | 2160 |
| catcttccaa aatcctatgg tacaagaagc tatacgaatg gggttcagtt tcaaggacat | 2220 |
| taagaaaata atggaggaaa aaattcagat atctgggagc aactataaat cacttgaggt | 2280 |
| tctggttgca gatctagtga atgctcagaa agacagtatg caagatgagt caagtcagac | 2340 |
| ttcattacag aaagagatta gtactgaaga gcagctaagg cgcctgcaag aggagaagct | 2400 |
| ttgcaaaatc tgtatggata gaaatattgc tatcgttttt gttccttgtg gacatctagt | 2460 |
| cacttgtaaa caatgtgctg aagcagttga caagtgtccc atgtgctaca cagtcattac | 2520 |
| tttcaagcaa aaaattttta tgtcttaatc taaagatctt attaaagcag aacttgttta | 2580 |
| ttgcagctta taatggttac aaataaagca atagcatcac aaatttcaca aataaagcat | 2640 |
| ttttttcact gcattctagt tgtggtttgt ccaaactcat caatgtatct tatcatgtct | 2700 |
| ggtcgactct agactcttcc gcttcctcgc tcactgactc gctgcgctcg gtcgttcggc | 2760 |
| tgcggcgagc ggtatcagct cactcaaagg cggtaatacg gttatccaca gaatcagggg | 2820 |
| ataacgcagg aaagaacatg tgagcaaaag gccagcaaaa ggccaggaac cgtaaaaagg | 2880 |
| ccgcgttgct ggcgtttttc cataggctcc gcccccctga cgagcatcac aaaaatcgac | 2940 |
| gctcaagtca gaggtggcga aacccgacag gactataaag ataccaggcg tttccccctg | 3000 |
| gaagctccct cgtgcgctct cctgttccga ccctgccgct taccggatac ctgtccgcct | 3060 |
| ttctcccttc gggaagcgtg gcgctttctc aatgctcacg ctgtaggtat ctcagttcgg | 3120 |
| tgtaggtcgt tcgctccaag ctgggctgtg tgcacgaacc ccccgttcag cccgaccgct | 3180 |
| gcgccttatc cggtaactat cgtcttgagt ccaacccggt aagacacgac ttatcgccac | 3240 |
| tggcagcagc cactggtaac aggattagca gagcgaggta tgtaggcggt gctacagagt | 3300 |
| tcttgaagtg gtggcctaac tacggctaca ctagaaggac agtatttggt atctgcgctc | 3360 |
| tgctgaagcc agttaccttc ggaaaaagag ttggtagctc ttgatccggc aaacaaacca | 3420 |
| ccgctggtag cggtggtttt tttgtttgca agcagcagat tacgcgcaga aaaaaaggat | 3480 |
| ctcaagaaga tcctttgatc ttttctacgg ggtctgacgc tcagtggaac gaaaactcac | 3540 |
| gttaagggat tttggtcatg agattatcaa aaaggatctt cacctagatc cttttaaatt | 3600 |
| aaaaatgaag ttttaaatca atctaaagta tatatgagta aacttggtct gacagttacc | 3660 |
| aatgcttaat cagtgaggca cctatctcag cgatctgtct atttcgttca tccatagttg | 3720 |
| cctgactccc cgtcgtgtag ataactacga tacgggaggg cttaccatct ggccccagtg | 3780 |
| ctgcaatgat accgcgagac ccacgctcac cggctccaga tttatcagca ataaaccagc | 3840 |
| cagccggaag ggccgagcgc agaagtggtc ctgcaacttt atccgcctcc atccagtcta | 3900 |
| ttaattgttg ccgggaagct agagtaagta gttcgccagt taatagtttg cgcaacgttg | 3960 |
| ttgccattgc tacaggcatc gtggtgtcac gctcgtcgtt tggtatggct tcattcagct | 4020 |
| ccggttccca acgatcaagg cgagttacat gatcccccat gttgtgcaaa aaagcggtta | 4080 |
| gctccttcgg tcctccgatc gttgtcagaa gtaagttggc cgcagtgtta tcactcatgg | 4140 |
| ttatggcagc actgcataat tctcttactg tcatgccatc cgtaagatgc ttttctgtga | 4200 |
| ctggtgagta ctcaaccaag tcattctgag aatagtgtat gcggcgaccg agttgctctt | 4260 |
| gcccggcgtc aatacgggat aataccgcgc cacatagcag aactttaaaa gtgctcatca | 4320 |
| ttggaaaacg ttcttcgggg cgaaaactct caaggatctt accgctgttg agatccagtt | 4380 |
| cgatgtaacc cactcgtgca cccaactgat cttcagcatc ttttactttc accagcgttt | 4440 |
| ctgggtgagc aaaaacagga aggcaaaatg ccgcaaaaaa gggaataagg gcgacacgga | 4500 |
| aatgttgaat actcatactc ttcttttttc aatattattg aagcatttat cagggttatt | 4560 |
| gtctcatgag cggatacata tttgaatgta tttagaaaaa taaacaaata ggggttccgc | 4620 |
| gcacatttcc ccgaaaagtg ccacctgacg tctaagaaac cattattatc atgacattaa | 4680 |
| cctataaaaa taggcgtatc acgaggcccc tttcgtctcg cgcgtttcgg tgatgacggt | 4740 |
| gaaaacctct gacacatgca gctcccggag acggtcacag cttgtctgta agcggatgcc | 4800 |
| gggagcagac aagcccgtca gggcgcgtca gcgggtgttg gcgggtgtcg gggctggctt | 4860 |
| aactatgcgg catcagagca gattgtactg agagtgcacc atatgcggtg tgaaataccg | 4920 |
| cacagatgcg taaggagaaa ataccgcatc aggaaattgt aaacgttaat attttgttaa | 4980 |
| aattcgcgtt aaatttttgt taaatcagct cattttttaa ccaataggcc gaaatcggca | 5040 |
| aaatccctta taaatcaaaa gaatagaccg agatagggtt gagtgttgtt ccagtttgga | 5100 |
| acaagagtcc actattaaag aacgtggact ccaacgtcaa agggcgaaaa accgtctatc | 5160 |
| agggcgatgg cccactacgt gaaccatcac cctaatcaag ttttttgggg tcgaggtgcc | 5220 |
| gtaaagcact aaatcggaac cctaaaggga gcccccgatt tagagcttga cggggaaagc | 5280 |
| cggcgaacgt ggcgagaaag gaagggaaga aagcgaaagg agcgggcgct agggcgctgg | 5340 |
| caagtgtagc ggtcacgctg cgcgtaacca ccacacccgc cgcgcttaat gcgccgctac | 5400 |
| agggcgcgtc gcgccattcg ccattcaggc tacgcaactg ttgggaaggg cgatcggtgc | 5460 |
| gggcctcttc gctattacgc cagctggcga aggggggatg tgctgcaagg cgattaagtt | 5520 |
| gggtaacgcc agggttttcc cagtcacgac gttgtaaaac gacggccagt gaatt | 5575 |
| SEQ ID NO: 74 | |
| atggacttca gcagaaatct ttatgatatt ggggaacaac tggacagtga agatctggcc | 60 |
| tccctcaagt tcctgagcct ggactacatt ccgcaaagga agcaagaacc catcaaggat | 120 |
| gccttgatgt tattccagag actccaggaa aagagaatgt tggaggaaag caatctgtcc | 180 |
| ttcctgaagg agctgctctt ccgaattaat agactggatt tgctgattac ctacctaaac | 240 |
| actagaaagg aggagatgga aagggaactt cagacaccag gcagggctca aatttctgcc | 300 |
| tacagggtca tgctctatca gatttcagaa gaagtgagca gatcagaatt gaggtctttt | 360 |
| aagtttcttt tgcaagagga aatctccaaa tgcaaactgg atgatgacat gaacctgctg | 420 |
| gatattttca tagagatgga gaagagggtc atcctgggag aaggaaagtt ggacatcctg | 480 |
| aaaagagtct gtgcccaaat caacaagagc ctgctgaaga taatcaacga ctatgaagaa | 540 |
| ttcagcaaag gggaggagtt gtgtggggta atgacaatct cggactctcc aagagaacag | 600 |
| gatagtgaat cacagacttt ggacaaagtt taccaaatga aaagcaaacc tcggggatac | 660 |
| tgtctgatca tcaacaatca caattttgca aaagcacggg agaaagtgcc caaacttcac | 720 |
| agcattaggg acaggaatgg aacacacttg gatgcagggg ctttgaccac gacctttgaa | 780 |
| gagcttcatt ttgagatcaa gccccacgat gactgcacag tagagcaaat ctatgagatt | 840 |
| ttgaaaatct accaactcat ggaccacagt aacatggact gcttcatctg ctgtatcctc | 900 |
| tcccatggag acaagggcat catctatggc actgatggac aggaggcccc catctatgag | 960 |
| ctgacatctc agttcactgg tttgaagtgc ccttcccttg ctggaaaacc caaagtgttt | 1020 |
| tttattcagg cttgtcaggg ggataactac cagaaaggta tacctgttga gactgattca | 1080 |
| gaggagcaac cctatttaga aatggattta tcatcacctc aaacgagata tatcccggat | 1140 |
| gaggctgact ttctgctggg gatggccact gtgaataact gtgtttccta ccgaaaccct | 1200 |
| gcagagggaa cctggtacat ccagtcactt tgccagagcc tgagagagcg atgtcctcga | 1260 |
| ggcgatgata ttctcaccat cctgactgaa gtgaactatg aagtaagcaa caaggatgac | 1320 |
| aagaaaaaca tggggaaaca gatgcctcag cctactttca cactaagaaa aaaacttgtc | 1380 |
| ttcccttctg attga | 1395 |
| SEQ ID NO: 75 | |
| Met Asp Phe Ser Arg Asn Leu Tyr Asp Ile Gly Glu Gln Leu Asp Ser | |
| 1 5 10 15 | |
| Glu Asp Leu Ala Ser Leu Lys Phe Leu Ser Leu Asp Tyr Ile Pro Gln | |
| 20 25 30 | |
| Arg Lys Gln Glu Pro Ile Lys Asp Ala Leu Met Leu Phe Gln Arg Leu | |
| 35 40 45 | |
| Gln Glu Lys Arg Met Leu Glu Glu Ser Asn Leu Ser Phe Leu Lys Glu | |
| 50 55 60 | |
| Leu Leu Phe Arg Ile Asn Arg Leu Asp Leu Leu Ile Thr Tyr Leu Asn | |
| 65 70 75 80 | |
| Thr Arg Lys Glu Glu Met Glu Arg Glu Leu Gln Thr Pro Gly Arg Ala | |
| 85 90 95 | |
| Gln Ile Ser Ala Tyr Arg Val Met Leu Tyr Gln Ile Ser Glu Glu Val | |
| 100 105 110 | |
| Ser Arg Ser Glu Leu Arg Ser Phe Lys Phe Leu Leu Gln Glu Glu Ile | |
| 115 120 125 | |
| Ser Lys Cys Lys Leu Asp Asp Asp Met Asn Leu Leu Asp Ile Phe Ile | |
| 130 135 140 | |
| Glu Met Glu Lys Arg Val Ile Leu Gly Glu Gly Lys Leu Asp Ile Leu | |
| 145 150 155 160 | |
| Lys Arg Val Cys Ala Gln Ile Asn Lys Ser Leu Leu Lys Ile Ile Asn | |
| 165 170 175 | |
| Asp Tyr Glu Glu Phe Ser Lys Gly Glu Glu Leu Cys Gly Val Met Thr | |
| 180 185 190 | |
| Ile Ser Asp Ser Pro Arg Glu Gln Asp Ser Glu Ser Gln Thr Leu Asp | |
| 195 200 205 | |
| Lys Val Tyr Gln Met Lys Ser Lys Pro Arg Gly Tyr Cys Leu Ile Ile | |
| 210 215 220 | |
| Asn Asn His Asn Phe Ala Lys Ala Arg Glu Lys Val Pro Lys Leu His | |
| 225 230 235 240 | |
| Ser Ile Arg Asp Arg Asn Gly Thr His Leu Asp Ala Gly Ala Leu Thr | |
| 245 250 255 | |
| Thr Thr Phe Glu Glu Leu His Phe Glu Ile Lys Pro His Asp Asp Cys | |
| 260 265 270 | |
| Thr Val Glu Gln Ile Tyr Glu Ile Leu Lys Ile Tyr Gln Leu Met Asp | |
| 275 280 285 | |
| His Ser Asn Met Asp Cys Phe Ile Cys Cys Ile Leu Ser His Gly Asp | |
| 290 295 300 | |
| Lys Gly Ile Ile Tyr Gly Thr Asp Gly Gln Glu Ala Pro Ile Tyr Glu | |
| 305 310 315 320 | |
| Leu Thr Ser Gln Phe Thr Gly Leu Lys Cys Pro Ser Leu Ala Gly Lys | |
| 325 330 335 | |
| Pro Lys Val Phe Phe Ile Gln Ala Cys Gln Gly Asp Asn Tyr Gln Lys | |
| 340 345 350 | |
| Gly Ile Pro Val Glu Thr Asp Ser Glu Glu Gln Pro Tyr Leu Glu Met | |
| 355 360 365 | |
| Asp Leu Ser Ser Pro Gln Thr Arg Tyr Ile Pro Asp Glu Ala Asp Phe | |
| 370 375 380 | |
| Leu Leu Gly Met Ala Thr Val Asn Asn Cys Val Ser Tyr Arg Asn Pro | |
| 385 390 395 400 | |
| Ala Glu Gly Thr Trp Tyr Ile Gln Ser Leu Cys Gln Ser Leu Arg Glu | |
| 405 410 415 | |
| Arg Cys Pro Arg Gly Asp Asp Ile Leu Thr Ile Leu Thr Glu Val Asn | |
| 420 425 430 | |
| Tyr Glu Val Ser Asn Lys Asp Asp Lys Lys Asn Met Gly Lys Gln Met | |
| 435 440 445 | |
| Pro Gln Pro Thr Phe Thr Leu Arg Lys Lys Leu Val Phe Pro Ser Asp | |
| 450 455 460 | |
| SEQ ID NO: 76 | |
| gtcgacttct gaggcggaaa gaaccagctg tggaatgtgt gtcagttagg gtgtggaaag | 60 |
| tccccaggct ccccagcagg cagaagtatg caaagcatgc atctcaatta gtcagcaacc | 120 |
| aggtgtggaa agtccccagg ctccccagca ggcagaagta tgcaaagcat gcatctcaat | 180 |
| tagtcagcaa ccatagtccc gcccctaact ccgcccatcc cgcccctaac tccgcccagt | 240 |
| tccgcccatt ctccgcccca tggctgacta atttttttta tttatgcaga ggccgaggcc | 300 |
| gcctcggcct ctgagctatt ccagaagtag tgaggaggct tttttggagg cctaggcttt | 360 |
| tgcaaaaagc tggatcgatc ctgagaactt cagggtgagt ttggggaccc ttgattgttc | 420 |
| tttctttttc gctattgtaa aattcatgtt atatggaggg ggcaaagttt tcagggtgtt | 480 |
| gtttagaatg ggaagatgtc ccttgtatca ccatggaccc tcatgataat tttgtttctt | 540 |
| tcactttcta ctctgttgac aaccattgtc tcctcttatt ttcttttcat tttctgtaac | 600 |
| tttttcgtta aactttagct tgcatttgta acgaattttt aaattcactt ttgtttattt | 660 |
| gtcagattgt aagtactttc tctaatcact tttttttcaa ggcaatcagg gtatattata | 720 |
| ttgtacttca gcacagtttt agagaacaat tgttataatt aaatgataag gtagaatatt | 780 |
| tctgcatata aattctggct ggcgtggaaa tattcttatt ggtagaaaca actacatcct | 840 |
| ggtcatcatc ctgcctttct ctttatggtt acaatgatat acactgtttg agatgaggat | 900 |
| aaaatactct gagtccaaac cgggcccctc tgctaaccat gttcatgcct tcttcttttt | 960 |
| cctacagctc ctgggcaacg tgctggttat tgtgctgtct catcattttg gcaaagaatt | 1020 |
| gtaatacgac tcactatagg gcgaattcat ggacttcagc agaaatcttt atgatattgg | 1080 |
| ggaacaactg gacagtgaag atctggcctc cctcaagttc ctgagcctgg actacattcc | 1140 |
| gcaaaggaag caagaaccca tcaaggatgc cttgatgtta ttccagagac tccaggaaaa | 1200 |
| gagaatgttg gaggaaagca atctgtcctt cctgaaggag ctgctcttcc gaattaatag | 1260 |
| actggatttg ctgattacct acctaaacac tagaaaggag gagatggaaa gggaacttca | 1320 |
| gacaccaggc agggctcaaa tttctgccta cagggtcatg ctctatcaga tttcagaaga | 1380 |
| agtgagcaga tcagaattga ggtcttttaa gtttcttttg caagaggaaa tctccaaatg | 1440 |
| caaactggat gatgacatga acctgctgga tattttcata gagatggaga agagggtcat | 1500 |
| cctgggagaa ggaaagttgg acatcctgaa aagagtctgt gcccaaatca acaagagcct | 1560 |
| gctgaagata atcaacgact atgaagaatt cagcaaaggg gaggagttgt gtggggtaat | 1620 |
| gacaatctcg gactctccaa gagaacagga tagtgaatca cagactttgg acaaagttta | 1680 |
| ccaaatgaaa agcaaacctc gggatactgt ctgatcatca acaatcacaa ttttgcaaaa | 1740 |
| gcacgggaga aagtgcccca aacttcacag cattagggac aggaatggaa cacacttgga | 1800 |
| tgcaggggct ttgaccacga cctttgaaga gcttcatttt gagatcaagc cccacgatga | 1860 |
| ctgcacagta gagcaaatct atgagatttt gaaaatctac caactcatgg accacagtaa | 1920 |
| catggactgc ttcatctgct gtatcctctc ccatggagac aagggcatca tctatggcac | 1980 |
| tgatggacag gaggccccca tctatgagct gacatctcag ttcactggtt tgaagtgccc | 2040 |
| ttcccttgct ggaaaaccca aagtgttttt tattcaggct tgtcaggggg ataactacca | 2100 |
| gaaaggtata cctgttgaga ctgattcaga ggagcaaccc tatttagaaa tggatttatc | 2160 |
| atcacctcaa acgagatata tcccggatga ggctgacttt ctgctgggga tggccactgt | 2220 |
| gaataactgt gtttcctacc gaaaccctgc agagggaacc tggtacatcc agtcactttg | 2280 |
| ccagagcctg agagagcgat gtcctcgagg cgatgatatt ctcaccatcc tgactgaagt | 2340 |
| gaactatgaa gtaagcaaca aggatgacaa gaaaaacatg gggaaacaga tgcctcagcc | 2400 |
| tactttcaca ctaagaaaaa aacttgtctt cccttctgat tgaggatcca gatcttatta | 2460 |
| aagcagaact tgtttattgc agcttataat ggttacaaat aaagcaatag catcacaaat | 2520 |
| ttcacaaata aagcattttt ttcactgcat tctagttgtg gtttgtccaa actcatcaat | 2580 |
| gtatcttatc atgtctggtc gactctagac tcttccgctt cctcgctcac tgactcgctg | 2640 |
| cgctcggtcg ttcggctgcg gcgagcggta tcagctcact caaaggcggt aatacggtta | 2700 |
| tccacagaat caggggataa cgcaggaaag aacatgtgag caaaaggcca gcaaaaggcc | 2760 |
| aggaaccgta aaaaggccgc gttgctggcg tttttccata ggctccgccc ccctgacgag | 2820 |
| catcacaaaa atcgacgctc aagtcagagg tggcgaaacc cgacaggact ataaagatac | 2880 |
| caggcgtttc cccctggaag ctccctcgtg cgctctcctg ttccgaccct gccgcttacc | 2940 |
| ggatacctgt ccgcctttct cccttcggga agcgtggcgc tttctcaatg ctcacgctgt | 3000 |
| aggtatctca gttcggtgta ggtcgttcgc tccaagctgg gctgtgtgca cgaacccccc | 3060 |
| gttcagcccg accgctgcgc cttatccggt aactatcgtc ttgagtccaa cccggtaaga | 3120 |
| cacgacttat cgccactggc agcagccact ggtaacagga ttagcagagc gaggtatgta | 3180 |
| ggcggtgcta cagagttctt gaagtggtgg cctaactacg gctacactag aaggacagta | 3240 |
| tttggtatct gcgctctgct gaagccagtt accttcggaa aaagagttgg tagctcttga | 3300 |
| tccggcaaac aaaccaccgc tggtagcggt ggtttttttg tttgcaagca gcagattacg | 3360 |
| cgcagaaaaa aaggatctca agaagatcct ttgatctttt ctacggggtc tgacgctcag | 3420 |
| tggaacgaaa actcacgtta agggattttg gtcatgagat tatcaaaaag gatcttcacc | 3480 |
| tagatccttt taaattaaaa atgaagtttt aaatcaatct aaagtatata tgagtaaact | 3540 |
| tggtctgaca gttaccaatg cttaatcagt gaggcaccta tctcagcgat ctgtctattt | 3600 |
| cgttcatcca tagttgcctg actccccgtc gtgtagataa ctacgatacg ggagggctta | 3660 |
| ccatctggcc ccagtgctgc aatgataccg cgagacccac gctcaccggc tccagattta | 3720 |
| tcagcaataa accagccagc cggaagggcc gagcgcagaa gtggtcctgc aactttatcc | 3780 |
| gcctccatcc agtctattaa ttgttgccgg gaagctagag taagtagttc gccagttaat | 3840 |
| agtttgcgca acgttgttgc cattgctaca ggcatcgtgg tgtcacgctc gtcgtttggt | 3900 |
| atggcttcat tcagctccgg ttcccaacga tcaaggcgag ttacatgatc ccccatgttg | 3960 |
| tgcaaaaaag cggttagctc cttcggtcct ccgatcgttg tcagaagtaa gttggccgca | 4020 |
| gtgttatcac tcatggttat ggcagcactg cataattctc ttactgtcat gccatccgta | 4080 |
| agatgctttt ctgtgactgg tgagtactca accaagtcat tctgagaata gtgtatgcgg | 4140 |
| cgaccgagtt gctcttgccc ggcgtcaata cgggataata ccgcgccaca tagcagaact | 4200 |
| ttaaaagtgc tcatcattgg aaaacgttct tcggggcgaa aactctcaag gatcttaccg | 4260 |
| ctgttgagat ccagttcgat gtaacccact cgtgcaccca actgatcttc agcatctttt | 4320 |
| actttcacca gcgtttctgg gtgagcaaaa acaggaaggc aaaatgccgc aaaaaaggga | 4380 |
| ataagggcga cacggaaatg ttgaatactc atactcttct tttttcaata ttattgaagc | 4440 |
| atttatcagg gttattgtct catgagcgga tacatatttg aatgtattta gaaaaataaa | 4500 |
| caaatagggg ttccgcgcac atttccccga aaagtgccac ctgacgtcta agaaaccatt | 4560 |
| attatcatga cattaaccta taaaaatagg cgtatcacga ggcccctttc gtctcgcgcg | 4620 |
| tttcggtgat gacggtgaaa acctctgaca catgcagctc ccggagacgg tcacagcttg | 4680 |
| tctgtaagcg gatgccggga gcagacaagc ccgtcagggc gcgtcagcgg gtgttggcgg | 4740 |
| gtgtcggggc tggcttaact atgcggcatc agagcagatt gtactgagag tgcaccatat | 4800 |
| gcggtgtgaa ataccgcaca gatgcgtaag gagaaaatac cgcatcagga aattgtaaac | 4860 |
| gttaatattt tgttaaaatt cgcgttaaat ttttgttaaa tcagctcatt ttttaaccaa | 4920 |
| taggccgaaa tcggcaaaat cccttataaa tcaaaagaat agaccgagat agggttgagt | 4980 |
| gttgttccag tttggaacaa gagtccacta ttaaagaacg tggactccaa cgtcaaaggg | 5040 |
| cgaaaaaccg tctatcaggg cgatggccca ctacgtgaac catcacccta atcaagtttt | 5100 |
| ttggggtcga ggtgccgtaa agcactaaat cggaacccta aagggagccc ccgatttaga | 5160 |
| gcttgacggg gaaagccggc gaacgtggcg agaaaggaag ggaagaaagc gaaaggagcg | 5220 |
| ggcgctaggg cgctggcaag tgtagcggtc acgctgcgcg taaccaccac acccgccgcg | 5280 |
| cttaatgcgc cgctacaggg cgcgtcgcgc cattcgccat tcaggctacg caactgttgg | 5340 |
| gaagggcgat cggtgcgggc ctcttcgcta ttacgccagc tggcgaaggg gggatgtgct | 5400 |
| gcaaggcgat taagttgggt aacgccaggg ttttcccagt cacgacgttg taaaacgacg | 5460 |
| gccagtgaat t | 5471 |
| SEQ ID NO: 77 | |
| atggcgcacg ctgggagaac agggtacgat aaccgggaga tagtgatgaa gtacatccat | 60 |
| tataagctgt cgcagagggg ctacgagtgg gatgcgggag atgtgggcgc cgcgcccccg | 120 |
| ggggccgccc ccgcaccggg catcttctcc tcccagcccg ggcacacgcc ccatccagcc | 180 |
| gcatcccggg acccggtcgc caggacctcg ccgctgcaga ccccggctgc ccccggcgcc | 240 |
| gccgcggggc ctgcgctcag cccggtgcca cctgtggtcc acctgaccct ccgccaggcc | 300 |
| ggcgacgact tctcccgccg ctaccgccgc gacttcgccg agatgtccag ccagctgcac | 360 |
| ctgacgccct tcaccgcgcg gggacgcttt gccacggtgg tggaggagct cttcagggac | 420 |
| ggggtgaact gggggaggat tgtggccttc tttgagttcg gtggggtcat gtgtgtggag | 480 |
| agcgtcaacc gggagatgtc gcccctggtg gacaacatcg ccctgtggat gactgagtac | 540 |
| ctgaaccggc acctgcacac ctggatccag gataacggag gctgggtagg tgcacttggt | 600 |
| gatgtgagtc tgggctga | 618 |
| SEQ ID NO: 78 | |
| Met Ala His Ala Gly Arg Thr Gly Tyr Asp Asn Arg Glu Ile Val Met | |
| 1 5 10 15 | |
| Lys Tyr Ile His Tyr Lys Leu Ser Gln Arg Gly Tyr Glu Trp Asp Ala | |
| 20 25 30 | |
| Gly Asp Val Gly Ala Ala Pro Pro Gly Ala Ala Pro Ala Pro Gly Ile | |
| 35 40 45 | |
| Phe Ser Ser Gln Pro Gly His Thr Pro His Pro Ala Ala Ser Arg Asp | |
| 50 55 60 | |
| Pro Val Ala Arg Thr Ser Pro Leu Gln Thr Pro Ala Ala Pro Gly Ala | |
| 65 70 75 80 | |
| Ala Ala Gly Pro Ala Leu Ser Pro Val Pro Pro Val Val His Leu Thr | |
| 85 90 95 | |
| Leu Arg Gln Ala Gly Asp Asp Phe Ser Arg Arg Tyr Arg Arg Asp Phe | |
| 100 105 110 | |
| Ala Glu Met Ser Ser Gln Leu His Leu Thr Pro Phe Thr Ala Arg Gly | |
| 115 120 125 | |
| Arg Phe Ala Thr Val Val Glu Glu Leu Phe Arg Asp Gly Val Asn Trp | |
| 130 135 140 | |
| Gly Arg Ile Val Ala Phe Phe Glu Phe Gly Gly Val Met Cys Val Glu | |
| 145 150 155 160 | |
| Ser Val Asn Arg Glu Met Ser Pro Leu Val Asp Asn Ile Ala Leu Trp | |
| 165 170 175 | |
| Met Thr Glu Tyr Leu Asn Arg His Leu His Thr Trp Ile Gln Asp Asn | |
| 180 185 190 | |
| Gly Gly Trp Val Gly Ala Leu Gly Asp Val Ser Leu Gly | |
| 195 200 205 | |
| SEQ ID NO: 79 | |
| gtcgacttct gaggcggaaa gaaccagctg tggaatgtgt gtcagttagg gtgtggaaag | 60 |
| tccccaggct ccccagcagg cagaagtatg caaagcatgc atctcaatta gtcagcaacc | 120 |
| aggtgtggaa agtccccagg ctccccagca ggcagaagta tgcaaagcat gcatctcaat | 180 |
| tagtcagcaa ccatagtccc gcccctaact ccgcccatcc cgcccctaac tccgcccagt | 240 |
| tccgcccatt ctccgcccca tggctgacta atttttttta tttatgcaga ggccgaggcc | 300 |
| gcctcggcct ctgagctatt ccagaagtag tgaggaggct tttttggagg cctaggcttt | 360 |
| tgcaaaaagc tggatcgatc ctgagaactt cagggtgagt ttggggaccc ttgattgttc | 420 |
| tttctttttc gctattgtaa aattcatgtt atatggaggg ggcaaagttt tcagggtgtt | 480 |
| gtttagaatg ggaagatgtc ccttgtatca ccatggaccc tcatgataat tttgtttctt | 540 |
| tcactttcta ctctgttgac aaccattgtc tcctcttatt ttcttttcat tttctgtaac | 600 |
| tttttcgtta aactttagct tgcatttgta acgaattttt aaattcactt ttgtttattt | 660 |
| gtcagattgt aagtactttc tctaatcact tttttttcaa ggcaatcagg gtatattata | 720 |
| ttgtacttca gcacagtttt agagaacaat tgttataatt aaatgataag gtagaatatt | 780 |
| tctgcatata aattctggct ggcgtggaaa tattcttatt ggtagaaaca actacatcct | 840 |
| ggtcatcatc ctgcctttct ctttatggtt acaatgatat acactgtttg agatgaggat | 900 |
| aaaatactct gagtccaaac cgggcccctc tgctaaccat gttcatgcct tcttcttttt | 960 |
| cctacagctc ctgggcaacg tgctggttat tgtgctgtct catcattttg gcaaagaatt | 1020 |
| gtaatacgac tcactatagg gcgaattcgg atccagatct atggcgcacg ctgggagaac | 1080 |
| agggtacgat aaccgggaga tagtgatgaa gtacatccat tataagctgt cgcagagggg | 1140 |
| ctacgagtgg gatgcgggag atgtgggcgc cgcgcccccg ggggccgccc ccgcaccggg | 1200 |
| catcttctcc tcccagcccg ggcacacgcc ccatccagcc gcatcccggg acccggtcgc | 1260 |
| caggacctcg ccgctgcaga ccccggctgc ccccggcgcc gccgcggggc ctgcgctcag | 1320 |
| cccggtgcca cctgtggtcc acctgaccct ccgccaggcc ggcgacgact tctcccgccg | 1380 |
| ctaccgccgc gacttcgccg agatgtccag ccagctgcac ctgacgccct tcaccgcgcg | 1440 |
| gggacgcttt gccacggtgg tggaggagct cttcagggac ggggtgaact gggggaggat | 1500 |
| tgtggccttc tttgagttcg gtggggtcat gtgtgtggag agcgtcaacc gggagatgtc | 1560 |
| gcccctggtg gacaacatcg ccctgtggat gactgagtac ctgaaccggc acctgcacac | 1620 |
| ctggatccag gataacggag gctgggtagg tgcacttggt gatgtgagtc tgggctgaag | 1680 |
| atcttattaa agcagaactt gtttattgca gcttataatg gttacaaata aagcaatagc | 1740 |
| atcacaaatt tcacaaataa agcatttttt tcactgcatt ctagttgtgg tttgtccaaa | 1800 |
| ctcatcaatg tatcttatca tgtctggtcg actctagact cttccgcttc ctcgctcact | 1860 |
| gactcgctgc gctcggtcgt tcggctgcgg cgagcggtat cagctcactc aaaggcggta | 1920 |
| atacggttat ccacagaatc aggggataac gcaggaaaga acatgtgagc aaaaggccag | 1980 |
| caaaaggcca ggaccgtaaa aaggccgcgt tgctggcgtt tttccatagg ctccgccccc | 2040 |
| ctgacgagca tcacaaaaat cgacgctcaa gtcagaggtg gcgaaacccg acaggactat | 2100 |
| aaagatacca ggcgtttccc cctggaagct ccctcgtgcg ctctcctgtt ccgaccctgc | 2160 |
| cgcttaccgg atacctgtcc gcctttctcc cttcgggaag cgtggcgctt tctcaatgct | 2220 |
| cacgctgtag gtatctcagt tcggtgtagg tcgttcgctc caagctgggc tgtgtgcacg | 2280 |
| aaccccccgt tcagcccgac cgctgcgcct tatccggtaa ctatcgtctt gagtccaacc | 2340 |
| cggtaagaca cgacttatcg ccactggcag cagccactgg taacaggatt agcagagcga | 2400 |
| ggtatgtagg cggtgctaca gagttcttga agtggtggcc taactacggc tacactagaa | 2460 |
| ggacagtatt tggtatctgc gctctgctga agccagttac cttcggaaaa agagttggta | 2520 |
| gctcttgatc cggcaaacaa accaccgctg gtagcggtgg tttttttgtt tgcaagcagc | 2580 |
| agattacgcg cagaaaaaaa ggatctcaag aagatccttt gatcttttct acggggtctg | 2640 |
| acgctcagtg gaacgaaaac tcacgttaag ggattttggt catgagatta tcaaaaagga | 2700 |
| tcttcaccta gatcctttta aattaaaaat gaagttttaa atcaatctaa agtatatatg | 2760 |
| agtaaacttg gtctgacagt taccaatgct taatcagtga ggcacctatc tcagcgatct | 2820 |
| gtctatttcg ttcatccata gttgcctgac tccccgtcgt gtagataact acgatacggg | 2880 |
| agggcttacc atctggcccc agtgctgcaa tgataccgcg agacccacgc tcaccggctc | 2940 |
| cagatttatc agcaataaac cagccagccg gaagggccga gcgcagaagt ggtcctgcaa | 3000 |
| ctttatccgc ctccatccag tctattaatt gttgccggga agctagagta agtagttcgc | 3060 |
| cagttaatag tttgcgcaac gttgttgcca ttgctacagg catcgtggtg tcacgctcgt | 3120 |
| cgtttggtat ggcttcattc agctccggtt cccaacgatc aaggcgagtt acatgatccc | 3180 |
| ccatgttgtg caaaaaagcg gttagctcct tcggtcctcc gatcgttgtc agaagtaagt | 3240 |
| tggccgcagt gttatcactc atggttatgg cagcactgca taattctctt actgtcatgc | 3300 |
| catccgtaag atgcttttct gtgactggtg agtactcaac caagtcattc tgagaatagt | 3360 |
| gtatgcggcg accgagttgc tcttgcccgg cgtcaatacg ggataatacc gcgccacata | 3420 |
| gcagaacttt aaaagtgctc atcattggaa aacgttcttc ggggcgaaaa ctctcaagga | 3480 |
| tcttaccgct gttgagatcc agttcgatgt aacccactcg tgcacccaac tgatcttcag | 3540 |
| catcttttac tttcaccagc gtttctgggt gagcaaaaac aggaaggcaa aatgccgcaa | 3600 |
| aaaagggaat aagggcgaca cggaaatgtt gaatactcat actcttcttt tttcaatatt | 3660 |
| attgaagcat ttatcagggt tattgtctca tgagcggata catatttgaa tgtatttaga | 3720 |
| aaaataaaca aataggggtt ccgcgcacat ttccccgaaa agtgccacct gacgtctaag | 3780 |
| aaaccattat tatcatgaca ttaacctata aaaataggcg tatcacgagg cccctttcgt | 3840 |
| ctcgcgcgtt tcggtgatga cggtgaaaac ctctgacaca tgcagctccc ggagacggtc | 3900 |
| acagcttgtc tgtaagcgga tgccgggagc agacaagccc gtcagggcgc gtcagcgggt | 3960 |
| gttggcgggt gtcggggctg gcttaactat gcggcatcag agcagattgt actgagagtg | 4020 |
| caccatatgc ggtgtgaaat accgcacaga tgcgtaagga gaaaataccg catcaggaaa | 4080 |
| ttgtaaacgt taatattttg ttaaaattcg cgttaaattt ttgttaaatc agctcatttt | 4140 |
| ttaaccaata ggccgaaatc ggcaaaatcc cttataaatc aaaagaatag accgagatag | 4200 |
| ggttgagtgt tgttccagtt tggaacaaga gtccactatt aaagaacgtg gactccaacg | 4260 |
| tcaaagggcg aaaaaccgtc tatcagggcg atggcccact acgtgaacca tcaccctaat | 4320 |
| caagtttttt ggggtcgagg tgccgtaaag cactaaatcg gaaccctaaa gggagccccc | 4380 |
| gatttagagc ttgacgggga aagccggcga acgtggcgag aaaggaaggg aagaaagcga | 4440 |
| aaggagcggg cgctagggcg ctggcaagtg tagcggtcac gctgcgcgta accaccacac | 4500 |
| ccgccgcgct taatgcgccg ctacagggcg cgtcgcgcca ttcgccattc aggctacgca | 4560 |
| actgttggga agggcgatcg gtgcgggcct cttcgctatt acgccagctg gcgaaggggg | 4620 |
| gatgtgctgc aaggcgatta agttgggtaa cgccagggtt ttcccagtca cgacgttgta | 4680 |
| aaacgacggc cagtgaatt | 4699 |
| SEQ ID NO: 80 | |
| gtcgacttct gaggcggaaa gaaccagctg tggaatgtgt gtcagttagg gtgtggaaag | 60 |
| tccccaggct ccccagcagg cagaagtatg caaagcatgc atctcaatta gtcagcaacc | 120 |
| aggtgtggaa agtccccagg ctccccagca ggcagaagta tgcaaagcat gcatctcaat | 180 |
| tagtcagcaa ccatagtccc gcccctaact ccgcccatcc cgcccctaac tccgcccagt | 240 |
| tccgcccatt ctccgcccca tggctgacta atttttttta tttatgcaga ggccgaggcc | 300 |
| gcctcggcct ctgagctatt ccagaagtag tgaggaggct tttttggagg cctaggcttt | 360 |
| tgcaaaaagc tggatcgatc ctgagaactt cagggtgagt ttggggaccc ttgattgttc | 420 |
| tttctttttc gctattgtaa aattcatgtt atatggaggg ggcaaagttt tcagggtgtt | 480 |
| gtttagaatg ggaagatgtc ccttgtatca ccatggaccc tcatgataat tttgtttctt | 540 |
| tcactttcta ctctgttgac aaccattgtc tcctcttatt ttcttttcat tttctgtaac | 600 |
| tttttcgtta aactttagct tgcatttgta acgaattttt aaattcactt ttgtttattt | 660 |
| gtcagattgt aagtactttc tctaatcact tttttttcaa ggcaatcagg gtatattata | 720 |
| ttgtacttca gcacagtttt agagaacaat tgttataatt aaatgataag gtagaatatt | 780 |
| tctgcatata aattctggct ggcgtggaaa tattcttatt ggtagaaaca actacatcct | 840 |
| ggtcatcatc ctgcctttct ctttatggtt acaatgatat acactgtttg agatgaggat | 900 |
| aaaatactct gagtccaaac cgggcccctc tgctaaccat gttcatgcct tcttcttttt | 960 |
| cctacagctc ctgggcaacg tgctggttat tgtgctgtct catcattttg gcaaagaatt | 1020 |
| gtaatacgac tcactatagg gcgaattcgg atccatggac ttcagcagaa atctttatga | 1080 |
| tattggggaa caactggaca gtgaagatct ggcctccctc aagttcctga gcctggacta | 1140 |
| cattccgcaa aggaagcaag aacccatcaa ggatgccttg atgttattcc agagactcca | 1200 |
| ggaaaagaga atgttggagg aaagcaatct gtccttcctg aaggagctgc tcttccgaat | 1260 |
| taatagactg gatttgctga ttacctacct aaacactaga aaggaggaga tggaaaggga | 1320 |
| acttcagaca ccaggcaggg ctcaaatttc tgcctacagg gtcatgctct atcagatttc | 1380 |
| agaagaagtg agcagatcag aattgaggtc ttttaagttt cttttgcaag aggaaatctc | 1440 |
| caaatgcaaa ctggatgatg acatgaacct gctggatatt ttcatagaga tggagaagag | 1500 |
| ggtcatcctg ggagaaggaa agttggacat cctgaaaaga gtctgtgccc aaatcaacaa | 1560 |
| gagcctgctg aagataatca acgactatga agaattcagc aaaggggagg agttgtgtgg | 1620 |
| ggtaatgaca atctcggact ctccaagaga acaggatagt gaatcacaga ctttggacaa | 1680 |
| agtttaccaa atgaaaagca aacctcgggg atactgtctg atcatcaaca atcacaattt | 1740 |
| tgcaaaagca cgggagaaag tgcccaaact tcacagcatt agggacagga atggaacaca | 1800 |
| cttggatgca ggggctttga ccacgacctt tgaagagctt cattttgaga tcaagcccca | 1860 |
| cgatgactgc acagtagagc aaatctatga gattttgaaa atctaccaac tcatggacca | 1920 |
| cagtaacatg gactgcttca tctgctgtat cctctcccat ggagacaagg gcatcatcta | 1980 |
| tggcactgat ggacaggagg cccccatcta tgagctgaca tctcagttca ctggtttgaa | 2040 |
| gtgcccttcc cttgctggaa aacccaaagt gttttttatt caggcttctc agggggataa | 2100 |
| ctaccagaaa ggtatacctg ttgagactga ttcagaggag caaccctatt tagaaatgga | 2160 |
| tttatcatca cctcaaacga gatatatccc ggatgaggct gactttctgc tggggatggc | 2220 |
| cactgtgaat aactgtgttt cctaccgaaa ccctgcagag ggaacctggt acatccagtc | 2280 |
| actttgccag agcctgagag agcgatgtcc tcgaggcgat gatattctca ccatcctgac | 2340 |
| tgaagtgaac tatgaagtaa gcaacaagga tgacaagaaa aacatgggga aacagatgcc | 2400 |
| tcagcctact ttcacactaa gaaaaaaact tgtcttccct tctgattgaa gatcttatta | 2460 |
| aagcagaact tgtttattgc agcttataat ggttacaaat aaagcaatag catcacaaat | 2520 |
| ttcacaaata aagcattttt ttcactgcat tctagttgtg gtttgtccaa actcatcaat | 2580 |
| gtatcttatc atgtctggtc gactctagac tcttccgctt cctcgctcac tgactcgctg | 2640 |
| cgctcggtcg ttcggctgcg gcgagcggta tcagctcact caaaggcggt aatacggtta | 2700 |
| tccacagaat caggggataa cgcaggaaag aacatgtgag caaaaggcca gcaaaaggcc | 2760 |
| aggaaccgta aaaaggccgc gttgctggcg tttttccata ggctccgccc ccctgacgag | 2820 |
| catcacaaaa atcgacgctc aagtcagagg tggcgaaacc cgacaggact ataaagatac | 2880 |
| caggcgtttc cccctggaag ctccctcgtg cgctctcctg ttccgaccct gccgcttacc | 2940 |
| ggatacctgt ccgcctttct cccttcggga agcgtggcgc tttctcaatg ctcacgctgt | 3000 |
| aggtatctca gttcggtgta ggtcgttcgc tccaagctgg gctgtgtgca cgaacccccc | 3060 |
| gttcagcccg accgctgcgc cttatccggt aactatcgtc ttgagtccaa cccggtaaga | 3120 |
| cacgacttat cgccactggc agcagccact ggtaacagga ttagcagagc gaggtatgta | 3180 |
| ggcggtgcta cagagttctt gaagtggtgg cctaactacg gctacactag aaggacagta | 3240 |
| tttggtatct gcgctctgct gaagccagtt accttcggaa aaagagttgg tagctcttga | 3300 |
| tccggcaaac aaaccaccgc tggtagcggt ggtttttttg tttgcaagca gcagattacg | 3360 |
| cgcagaaaaa aaggatctca agaagatcct ttgatctttt ctacggggtc tgacgctcag | 3420 |
| tggaacgaaa actcacgtta agggattttg gtcatgagat tatcaaaaag gatcttcacc | 3480 |
| tagatccttt taaattaaaa atgaagtttt aaatcaatct aaagtatata tgagtaaact | 3540 |
| tggtctgaca gttaccaatg cttaatcagt gaggcaccta tctcagcgat ctgtctattt | 3600 |
| cgttcatcca tagttgcctg actccccgtc gtgtagataa ctacgatacg ggagggctta | 3660 |
| ccatctggcc ccagtgctgc aatgataccg cgagacccac gctcaccggc tccagattta | 3720 |
| tcagcaataa accagccagc cggaagggcc gagcgcagaa gtggtcctgc aactttatcc | 3780 |
| gcctccatcc agtctattaa ttgttgccgg gaagctagag taagtagttc gccagttaat | 3840 |
| agtttgcgca acgttgttgc cattgctaca ggcatcgtgg tgtcacgctc gtcgtttggt | 3900 |
| atggcttcat tcagctccgg ttcccaacga tcaaggcgag ttacatgatc ccccatgttg | 3960 |
| tgcaaaaaag cggttagctc cttcggtcct ccgatcgttg tcagaagtaa gttggccgca | 4020 |
| gtgttatcac tcatggttat ggcagcactg cataattctc ttactgtcat gccatccgta | 4080 |
| agatgctttt ctgtgactgg tgagtactca accaagtcat tctgagaata gtgtatgcgg | 4140 |
| cgaccgagtt gctcttgccc ggcgtcaata cgggataata ccgcgccaca tagcagaact | 4200 |
| ttaaaagtgc tcatcattgg aaaacgttct tcggggcgaa aactctcaag gatcttaccg | 4260 |
| ctgttgagat ccagttcgat gtaacccact cgtgcaccca actgatcttc agcatctttt | 4320 |
| actttcacca gcgtttctgg gtgagcaaaa acaggaaggc aaaatgccgc aaaaaaggga | 4380 |
| ataagggcga cacggaaatg ttgaatactc atactcttct tttttcaata ttattgaagc | 4440 |
| atttatcagg gttattgtct catgagcgga tacatatttg aatgtattta gaaaaataaa | 4500 |
| caaatagggg ttccgcgcac atttccccga aaagtgccac ctgacgtcta agaaaccatt | 4560 |
| attatcatga cattaaccta taaaaatagg cgtatcacga ggcccctttc gtctcgcgcg | 4620 |
| tttcggtgat gacggtgaaa acctctgaca catgcagctc ccggagacgg tcacagcttg | 4680 |
| tctgtaagcg gatgccggga gcagacaagc ccgtcagggc gcgtcagcgg gtgttggcgg | 4740 |
| gtgtcggggc tggcttaact atgcggcatc agagcagatt gtactgagag tgcaccatat | 4800 |
| gcggtgtgaa ataccgcaca gatgcgtaag gagaaaatac cgcatcagga aattgtaaac | 4860 |
| gttaatattt tgttaaaatt cgcgttaaat ttttgttaaa tcagctcatt ttttaaccaa | 4920 |
| taggccgaaa tcggcaaaat cccttataaa tcaaaagaat agaccgagat agggttgagt | 4980 |
| gttgttccag tttggaacaa gagtccacta ttaaagaacg tggactccaa cgtcaaaggg | 5040 |
| cgaaaaaccg tctatcaggg cgatggccca ctacgtgaac catcacccta atcaagtttt | 5100 |
| ttggggtcga ggtgccgtaa agcactaaat cggaacccta aagggagccc ccgatttaga | 5160 |
| gcttgacggg gaaagccggc gaacgtggcg agaaaggaag ggaagaaagc gaaaggagcg | 5220 |
| ggcgctaggg cgctggcaag tgtagcggtc acgctgcgcg taaccaccac acccgccgcg | 5280 |
| cttaatgcgc cgctacaggg cgcgtcgcgc cattcgccat tcaggctacg caactgttgg | 5340 |
| gaagggcgat cggtgcgggc ctcttcgcta ttacgccagc tggcgaaggg gggatgtgct | 5400 |
| gcaaggcgat taagttgggt aacgccaggg ttttcccagt cacgacgttg taaaacgacg | 5460 |
| gccagtgaat t | 5471 |
| SEQ ID NO: 81 | |
| Met Asp Phe Ser Arg Asn Leu Tyr Asp Ile Gly Glu Gln Leu Asp Ser | |
| 1 5 10 15 | |
| Glu Asp Leu Ala Ser Leu Lys Phe Leu Ser Leu Asp Tyr Ile Pro Gln | |
| 20 25 30 | |
| Arg Lys Gln Glu Pro Ile Lys Asp Ala Leu Met Leu Phe Gln Arg Leu | |
| 35 40 45 | |
| Gln Glu Lys Arg Met Leu Glu Glu Ser Asn Leu Ser Phe Leu Lys Glu | |
| 50 55 60 | |
| Leu Leu Phe Arg Ile Asn Arg Leu Asp Leu Leu Ile Thr Tyr Leu Asn | |
| 65 70 75 80 | |
| Thr Arg Lys Glu Glu Met Glu Arg Glu Leu Gln Thr Pro Gly Arg Ala | |
| 85 90 95 | |
| Gln Ile Ser Ala Tyr Arg Val Met Leu Tyr Gln Ile Ser Glu Glu Val | |
| 100 105 110 | |
| Ser Arg Ser Glu Leu Arg Ser Phe Lys Phe Leu Leu Gln Glu Glu Ile | |
| 115 120 125 | |
| Ser Lys Cys Lys Leu Asp Asp Asp Met Asn Leu Leu Asp Ile Phe Ile | |
| 130 135 140 | |
| Glu Met Glu Lys Arg Val Ile Leu Gly Glu Gly Lys Leu Asp Ile Leu | |
| 145 150 155 160 | |
| Lys Arg Val Cys Ala Gln Ile Asn Lys Ser Leu Leu Lys Ile Ile Asn | |
| 165 170 175 | |
| Asp Tyr Glu Glu Phe Ser Lys Gly Glu Glu Leu Cys Gly Val Met Thr | |
| 180 185 190 | |
| Ile Ser Asp Ser Pro Arg Glu Gln Asp Ser Glu Ser Gln Thr Leu Asp | |
| 195 200 205 | |
| Lys Val Tyr Gln Met Lys Ser Lys Pro Arg Gly Tyr Cys Leu Ile Ile | |
| 210 215 220 | |
| Asn Asn His Asn Phe Ala Lys Ala Arg Glu Lys Val Pro Lys Leu His | |
| 225 230 235 240 | |
| Ser Ile Arg Asp Arg Asn Gly Thr His Leu Asp Ala Gly Ala Leu Thr | |
| 245 250 255 | |
| Thr Thr Phe Glu Glu Leu His Phe Glu Ile Lys Pro His Asp Asp Cys | |
| 260 265 270 | |
| Thr Val Glu Gln Ile Tyr Glu Ile Leu Lys Ile Tyr Gln Leu Met Asp | |
| 275 280 285 | |
| His Ser Asn Met Asp Cys Phe Ile Cys Cys Ile Leu Ser His Gly Asp | |
| 290 295 300 | |
| Lys Gly Ile Ile Tyr Gly Thr Asp Gly Gln Glu Ala Pro Ile Tyr Glu | |
| 305 310 315 320 | |
| Leu Thr Ser Gln Phe Thr Gly Leu Lys Cys Pro Ser Leu Ala Gly Lys | |
| 325 330 335 | |
| Pro Lys Val Phe Phe Ile Gln Ala Ser Gln Gly Asp Asn Tyr Gln Lys | |
| 340 345 350 | |
| Gly Ile Pro Val Glu Thr Asp Ser Glu Glu Gln Pro Tyr Leu Glu Met | |
| 355 360 365 | |
| Asp Leu Ser Ser Pro Gln Thr Arg Tyr Ile Pro Asp Glu Ala Asp Phe | |
| 370 375 380 | |
| Leu Leu Gly Met Ala Thr Val Asn Asn Cys Val Ser Tyr Arg Asn Pro | |
| 385 390 395 400 | |
| Ala Glu Gly Thr Trp Tyr Ile Gln Ser Leu Cys Gln Ser Leu Arg Glu | |
| 405 410 415 | |
| Arg Cys Pro Arg Gly Asp Asp Ile Leu Thr Ile Leu Thr Glu Val Asn | |
| 420 425 430 | |
| Tyr Glu Val Ser Asn Lys Asp Asp Lys Lys Asn Met Gly Lys Gln Met | |
| 435 440 445 | |
| Pro Gln Pro Thr Phe Thr Leu Arg Lys Lys Leu Val Phe Pro Ser Asp | |
| 450 455 460 | |
| SEQ ID NO: 82 | |
| gtcgacttct gaggcggaaa gaaccagctg tggaatgtgt gtcagttagg gtgtggaaag | 60 |
| tccccaggct ccccagcagg cagaagtatg caaagcatgc atctcaatta gtcagcaacc | 120 |
| aggtgtggaa agtccccagg ctccccagca ggcagaagta tgcaaagcat gcatctcaat | 180 |
| tagtcagcaa ccatagtccc gcccctaact ccgcccatcc cgcccctaac tccgcccagt | 240 |
| tccgcccatt ctccgcccca tggctgacta atttttttta tttatgcaga ggccgaggcc | 300 |
| gcctcggcct ctgagctatt ccagaagtag tgaggaggct tttttggagg cctaggcttt | 360 |
| tgcaaaaagc tggatcgatc ctgagaactt cagggtgagt ttggggaccc ttgattgttc | 420 |
| tttctttttc gctattgtaa aattcatgtt atatggaggg ggcaaagttt tcagggtgtt | 480 |
| gtttagaatg ggaagatgtc ccttgtatca ccatggaccc tcatgataat tttgtttctt | 540 |
| tcactttcta ctctgttgac aaccattgtc tcctcttatt ttcttttcat tttctgtaac | 600 |
| tttttcgtta aactttagct tgcatttgta acgaattttt aaattcactt ttgtttattt | 660 |
| gtcagattgt aagtactttc tctaatcact tttttttcaa ggcaatcagg gtatattata | 720 |
| ttgtacttca gcacagtttt agagaacaat tgttataatt aaatgataag gtagaatatt | 780 |
| tctgcatata aattctggct ggcgtggaaa tattcttatt ggtagaaaca actacatcct | 840 |
| ggtcatcatc ctgcctttct ctttatggtt acaatgatat acactgtttg agatgaggat | 900 |
| aaaatactct gagtccaaac cgggcccctc tgctaaccat gttcatgcct tcttcttttt | 960 |
| cctacagctc ctgggcaacg tgctggttat tgtgctgtct catcattttg gcaaagaatt | 1020 |
| gtaatacgac tcactatagg gcgaattcgg atccatggac gaagcggatc ggcggctcct | 1080 |
| gcggcggtgc cggctgcggc tggtggaaga gctgcaggtg gaccagctct gggacgccct | 1140 |
| gctgagccgc gagctgttca ggccccatat gatcgaggac atccagcggg caggctctgg | 1200 |
| atctcggcgg gatcaggcca ggcagctgat catagatctg gagactcgag ggagtcaggc | 1260 |
| tcttcctttg ttcatctcct gcttagagga cacaggccag gacatgctgg cttcgtttct | 1320 |
| gcgaactaac aggcaagcag caaagttgtc gaagccaacc ctagaaaacc ttaccccagt | 1380 |
| ggtgctcaga ccagagattc gcaaaccaga ggttctcaga ccggaaacac ccagaccagt | 1440 |
| ggacattggt tctggaggat ttggtgatgt cggtgctctt gagagtttga ggggaaatgc | 1500 |
| agatttggct tacatcctga gcatggagcc ctgtggccac tgcctcatta tcaacaatgt | 1560 |
| gaacttctgc cgtgagtccg ggctccgcac ccgcactggc tccaacatcg actgtgagaa | 1620 |
| gttgcggcgt cgcttctcct cgctgcattt catggtggag gtgaagggcg acctgactgc | 1680 |
| caagaaaatg gtgctggctt tgctggagct ggcgcagcag gaccacggtg ctctggactg | 1740 |
| ctgcgtggtg gtcattctct ctcacggctg tcaggccagc cacctgcagt tcccaggggc | 1800 |
| tgtctacggc acagatggat gccctgtgtc ggtcgagaag attgtgaaca tcttcaatgg | 1860 |
| gaccagctgc cccagcctgg gagggaagcc caagctcttt ttcatccagg cctctggtgg | 1920 |
| ggagcagaaa gaccatgggt ttgaggtggc ctccacttcc cctgaagacg agtcccctgg | 1980 |
| cagtaacccc gagccagatg ccaccccgtt ccaggaaggt ttgaggacct tcgaccagct | 2040 |
| ggacgccata tctagtttgc ccacacccag tgacatcttt gtgtcctact ctactttccc | 2100 |
| aggttttgtt tcctggaggg accccaagag tggctcctgg tacgttgaga ccctggacga | 2160 |
| catctttgag cagtgggctc actctgaaga cctgcagtcc ctcctgctta gggtcgctaa | 2220 |
| tgctgtttcg gtgaaaggga tttataaaca gatgcctggt tgctttaatt tcctccggaa | 2280 |
| aaaacttttc tttaaaacat cataaagatc ttattaaagc agaacttgtt tattgcagct | 2340 |
| tataatggtt acaaataaag caatagcatc acaaatttca caaataaagc atttttttca | 2400 |
| ctgcattcta gttgtggttt gtccaaactc atcaatgtat cttatcatgt ctggtcgact | 2460 |
| ctagactctt ccgcttcctc gctcactgac tcgctgcgct cggtcgttcg gctgcggcga | 2520 |
| gcggtatcag ctcactcaaa ggcggtaata cggttatcca cagaatcagg ggataacgca | 2580 |
| ggaaagaaca tgtgagcaaa aggccagcaa aaggccagga accgtaaaaa ggccgcgttg | 2640 |
| ctggcgtttt tccataggct ccgcccccct gacgagcatc acaaaaatcg acgctcaagt | 2700 |
| cagaggtggc gaaacccgac aggactataa agataccagg cgtttccccc tggaagctcc | 2760 |
| ctcgtgcgct ctcctgttcc gaccctgccg cttaccggat acctgtccgc ctttctccct | 2820 |
| tcgggaagcg tggcgctttc tcaatgctca cgctgtaggt atctcagttc ggtgtaggtc | 2880 |
| gttcgctcca agctgggctg tgtgcacgaa ccccccgttc agcccgaccg ctgcgcctta | 2940 |
| tccggtaact atcgtcttga gtccaacccg gtaagacacg acttatcgcc actggcagca | 3000 |
| gccactggta acaggattag cagagcgagg tatgtaggcg gtgctacaga gttcttgaag | 3060 |
| tggtggccta actacggcta cactagaagg acagtatttg gtatctgcgc tctgctgaag | 3120 |
| ccagttacct tcggaaaaag agttggtagc tcttgatccg gcaaacaaac caccgctggt | 3180 |
| agcggtggtt tttttgtttg caagcagcag attacgcgca gaaaaaaagg atctcaagaa | 3240 |
| gatcctttga tcttttctac ggggtctgac gctcagtgga acgaaaactc acgttaaggg | 3300 |
| attttggtca tgagattatc aaaaaggatc ttcacctaga tccttttaaa ttaaaaatga | 3360 |
| agttttaaat caatctaaag tatatatgag taaacttggt ctgacagtta ccaatgctta | 3420 |
| atcagtgagg cacctatctc agcgatctgt ctatttcgtt catccatagt tgcctgactc | 3480 |
| cccgtcgtgt agataactac gatacgggag ggcttaccat ctggccccag tgctgcaatg | 3540 |
| ataccgcgag acccacgctc accggctcca gatttatcag caataaacca gccagccgga | 3600 |
| agggccgagc gcagaagtgg tcctgcaact ttatccgcct ccatccagtc tattaattgt | 3660 |
| tgccgggaag ctagagtaag tagttcgcca gttaatagtt tgcgcaacgt tgttgccatt | 3720 |
| gctacaggca tcgtggtgtc acgctcgtcg tttggtatgg cttcattcag ctccggttcc | 3780 |
| caacgatcaa ggcgagttac atgatccccc atgttgtgca aaaaagcggt tagctccttc | 3840 |
| ggtcctccga tcgttgtcag aagtaagttg gccgcagtgt tatcactcat ggttatggca | 3900 |
| gcactgcata attctcttac tgtcatgcca tccgtaagat gcttttctgt gactggtgag | 3960 |
| tactcaacca agtcattctg agaatagtgt atgcggcgac cgagttgctc ttgcccggcg | 4020 |
| tcaatacggg ataataccgc gccacatagc agaactttaa aagtgctcat cattggaaaa | 4080 |
| cgttcttcgg ggcgaaaact ctcaaggatc ttaccgctgt tgagatccag ttcgatgtaa | 4140 |
| cccactcgtg cacccaactg atcttcagca tcttttactt tcaccagcgt ttctgggtga | 4200 |
| gcaaaaacag gaaggcaaaa tgccgcaaaa aagggaataa gggcgacacg gaaatgttga | 4260 |
| atactcatac tcttcttttt tcaatattat tgaagcattt atcagggtta ttgtctcatg | 4320 |
| agcggataca tatttgaatg tatttagaaa aataaacaaa taggggttcc gcgcacattt | 4380 |
| ccccgaaaag tgccacctga cgtctaagaa accattatta tcatgacatt aacctataaa | 4440 |
| aataggcgta tcacgaggcc cctttcgtct cgcgcgtttc ggtgatgacg gtgaaaacct | 4500 |
| ctgacacatg cagctcccgg agacggtcac agcttgtctg taagcggatg ccgggagcag | 4560 |
| acaagcccgt cagggcgcgt cagcgggtgt tggcgggtgt cggggctggc ttaactatgc | 4620 |
| ggcatcagag cagattgtac tgagagtgca ccatatgcgg tgtgaaatac cgcacagatg | 4680 |
| cgtaaggaga aaataccgca tcaggaaatt gtaaacgtta atattttgtt aaaattcgcg | 4740 |
| ttaaattttt gttaaatcag ctcatttttt aaccaatagg ccgaaatcgg caaaatccct | 4800 |
| tataaatcaa aagaatagac cgagataggg ttgagtgttg ttccagtttg gaacaagagt | 4860 |
| ccactattaa agaacgtgga ctccaacgtc aaagggcgaa aaaccgtcta tcagggcgat | 4920 |
| ggcccactac gtgaaccatc accctaatca agttttttgg ggtcgaggtg ccgtaaagca | 4980 |
| ctaaatcgga accctaaagg gagcccccga tttagagctt gacggggaaa gccggcgaac | 5040 |
| gtggcgagaa aggaagggaa gaaagcgaaa ggagcgggcg ctagggcgct ggcaagtgta | 5100 |
| gcggtcacgc tgcgcgtaac caccacaccc gccgcgctta atgcgccgct acagggcgcg | 5160 |
| tcgcgccatt cgccattcag gctacgcaac tgttgggaag ggcgatcggt gcgggcctct | 5220 |
| tcgctattac gccagctggc gaagggggga tgtgctgcaa ggcgattaag ttgggtaacg | 5280 |
| ccagggtttt cccagtcacg acgttgtaaa acgacggcca gtgaatt | 5327 |
| SEQ ID NO: 83 | |
| Met Asp Glu Ala Asp Arg Arg Leu Leu Arg Arg Cys Arg Leu Arg Leu | |
| 1 5 10 15 | |
| Val Glu Glu Leu Gln Val Asp Gln Leu Trp Asp Ala Leu Leu Ser Arg | |
| 20 25 30 | |
| Glu Leu Phe Arg Pro His Met Ile Glu Asp Ile Gln Arg Ala Gly Ser | |
| 35 40 45 | |
| Gly Ser Arg Arg Asp Gln Ala Arg Gln Leu Ile Ile Asp Leu Glu Thr | |
| 50 55 60 | |
| Arg Gly Ser Gln Ala Leu Pro Leu Phe Ile Ser Cys Leu Glu Asp Thr | |
| 65 70 75 80 | |
| Gly Gln Asp Met Leu Ala Ser Phe Leu Arg Thr Asn Arg Gln Ala Ala | |
| 85 90 95 | |
| Lys Leu Ser Lys Pro Thr Leu Glu Asn Leu Thr Pro Val Val Leu Arg | |
| 100 105 110 | |
| Pro Glu Ile Arg Lys Pro Glu Val Leu Arg Pro Glu Thr Pro Arg Pro | |
| 115 120 125 | |
| Val Asp Ile Gly Ser Gly Gly Phe Gly Asp Val Gly Ala Leu Glu Ser | |
| 130 135 140 | |
| Leu Arg Gly Asn Ala Asp Leu Ala Tyr Ile Leu Ser Met Glu Pro Cys | |
| 145 150 155 160 | |
| Gly His Cys Leu Ile Ile Asn Asn Val Asn Phe Cys Arg Glu Ser Gly | |
| 165 170 175 | |
| Leu Arg Thr Arg Thr Gly Ser Asn Ile Asp Cys Glu Lys Leu Arg Arg | |
| 180 185 190 | |
| Arg Phe Ser Ser Leu His Phe Met Val Glu Val Lys Gly Asp Leu Thr | |
| 195 200 205 | |
| Ala Lys Lys Met Val Leu Ala Leu Leu Glu Leu Ala Gln Gln Asp His | |
| 210 215 220 | |
| Gly Ala Leu Asp Cys Cys Val Val Val Ile Leu Ser His Gly Cys Gln | |
| 225 230 235 240 | |
| Ala Ser His Leu Gln Phe Pro Gly Ala Val Tyr Gly Thr Asp Gly Cys | |
| 245 250 255 | |
| Pro Val Ser Val Glu Lys Ile Val Asn Ile Phe Asn Gly Thr Ser Cys | |
| 260 265 270 | |
| Pro Ser Leu Gly Gly Lys Pro Lys Leu Phe Phe Ile Gln Ala Ser Gly | |
| 275 280 285 | |
| Gly Glu Gln Lys Asp His Gly Phe Glu Val Ala Ser Thr Ser Pro Glu | |
| 290 295 300 | |
| Asp Glu Ser Pro Gly Ser Asn Pro Glu Pro Asp Ala Thr Pro Phe Gln | |
| 305 310 315 320 | |
| Glu Gly Leu Arg Thr Phe Asp Gln Leu Asp Ala Ile Ser Ser Leu Pro | |
| 325 330 335 | |
| Thr Pro Ser Asp Ile Phe Val Ser Tyr Ser Thr Phe Pro Gly Phe Val | |
| 340 345 350 | |
| Ser Trp Arg Asp Pro Lys Ser Gly Ser Trp Tyr Val Glu Thr Leu Asp | |
| 355 360 365 | |
| Asp Ile Phe Glu Gln Trp Ala His Ser Glu Asp Leu Gln Ser Leu Leu | |
| 370 375 380 | |
| Leu Arg Val Ala Asn Ala Val Ser Val Lys Gly Ile Tyr Lys Gln Met | |
| 385 390 395 400 | |
| Pro Gly Cys Phe Asn Phe Leu Arg Lys Lys Leu Phe Phe Lys Thr Ser | |
| 405 410 415 | |
| SEQ ID NO: 84 | |
| gaattccggg ctggattgag aagccgcaac tgtgactctg catcatgaat actctgtctg | 60 |
| aaggaaatgg cacctttgcc atccatcttt tgaagatgct atgtcaaagc aacccttcca | 120 |
| aaaatgtatg ttattctcct gcgagcatct cctctgctct agctatggtt ctcttgggtg | 180 |
| caaagggaca gacggcagtc cagatatctc aggcacttgg tttgaataaa gaggaaggca | 240 |
| tccatcaggg tttccagttg cttctcagga agctgaacaa gccagacaga aagtactctc | 300 |
| ttagagtggc caacaggctc tttgcagaca aaacttgtga agtcctccaa acctttaagg | 360 |
| agtcctctct tcacttctat gactcagaga tggagcagct ctcctttgct gaagaagcag | 420 |
| aggtgtccag gcaacacata aacacatggg tctccaaaca aactgaaggt aaaattccag | 480 |
| agttgttgtc aggtggctcc gtcgattcag aaaccaggct ggttctcatc aatgccttat | 540 |
| attttaaagg aaagtggcat caaccattta acaaagagta cacaatggac atgcccttta | 600 |
| aaataaacaa ggatgagaaa aggccagtgc agatgatgtg tcgtgaagac acatataacc | 660 |
| tcgcctatgt gaaggaggtg caggcgcaag tgctggtgat gccatatgaa ggaatggagc | 720 |
| tgagcttggt ggttctgctc ccagatgagg gtgtggacct cagcaaggtg gaaaacaatc | 780 |
| tcacttttga gaagttaaca gcctggatgg aagcagattt tatgaagagc actgatgttg | 840 |
| aggttttcct tccaaaattt aaactccaag aggattatga catggagtct ctgtttcagc | 900 |
| gcttgggagt ggtggatgtc ttccaagagg acaaggctga cttatcagga atgtctccag | 960 |
| agagaaacct gtgtgtgtcc aagtttgttc accagagtgt agtggagatc aatgaggaag | 1020 |
| gcacagaggc tgcagcagcc tctgccatca tagaattttg ctgtgcctct tctgtcccaa | 1080 |
| cattctgtgc tgaccacccc ttccttttct tcatcaggca caacaaagca aacagcatcc | 1140 |
| tgttctgtgg caggttctca tctccataaa gacacatata ctacacaggg agagttctct | 1200 |
| cttcagtatc cctaccactc ctacagctct gtcaagatgg gcaagtaggg ggaagtcatg | 1260 |
| ttctaagatg aagacacttt ccttctctgt cagcctgatc ttataatgcc tgcattcaac | 1320 |
| tctccctgtc ttgaatgcat ctatgccctt taccaggtta tgtctaatga tgccaaatac | 1380 |
| cttctgctat gctattgatt gatagcctag ccagtaattt atagccagtt agaactgact | 1440 |
| tgactgtgca agaatgctat aatggagcta gagagaaggc acaaacacta ggaaaggttg | 1500 |
| ctgtttttgc agaggacaca gggacatttc ccaccactca catggctgct tacaacctct | 1560 |
| ggaaattcca gtttctgtcc atgacttgat tcctttcttt ggcttctact ggctccagca | 1620 |
| tcctgcacat acatgtatcg tcattcagtt acacacaaac aagtaaaatt ttaaaaataa | 1680 |
| ataaaaattt aaagagagag tctaaaattt tagtaatggt tagataatag ctgctattgt | 1740 |
| gcctttttca ggttttaatg tcattattct tgtgtataaa gtcaataatt tataggaaaa | 1800 |
| catcagtgcc ccggaattc | 1819 |
| SEQ ID NO: 85 | |
| Met Asn Thr Leu Ser Glu Gly Asn Gly Thr Phe Ala Ile His Leu Leu | |
| 1 5 10 15 | |
| Lys Met Leu Cys Gln Ser Asn Pro Ser Lys Asn Val Cys Tyr Ser Pro | |
| 20 25 30 | |
| Ala Ser Ile Ser Ser Ala Leu Ala Met Val Leu Leu Gly Ala Lys Gly | |
| 35 40 45 | |
| Gln Thr Ala Val Gln Ile Ser Gln Ala Leu Gly Leu Asn Lys Glu Glu | |
| 50 55 60 | |
| Gly Ile His Gln Gly Phe Gln Leu Leu Leu Arg Lys Leu Asn Lys Pro | |
| 65 70 75 80 | |
| Asp Arg Lys Tyr Ser Leu Arg Val Ala Asn Arg Leu Phe Ala Asp Lys | |
| 85 90 95 | |
| Thr Cys Glu Val Leu Gln Thr Phe Lys Glu Ser Ser Leu His Phe Tyr | |
| 100 105 110 | |
| Asp Ser Glu Met Glu Gln Leu Ser Phe Ala Glu Glu Ala Glu Val Ser | |
| 115 120 125 | |
| Arg Gln His Ile Asn Thr Trp Val Ser Lys Gln Thr Glu Gly Lys Ile | |
| 130 135 140 | |
| Pro Glu Leu Leu Ser Gly Gly Ser Val Asp Ser Glu Thr Arg Leu Val | |
| 145 150 155 160 | |
| Leu Ile Asn Ala Leu Tyr Phe Lys Gly Lys Trp His Gln Pro Phe Met | |
| 165 170 175 | |
| Lys Glu Tyr Thr Met Asp Met Pro Phe Lys Ile Asn Lys Asp Glu Lys | |
| 180 185 190 | |
| Arg Pro Val Gln Met Met Cys Arg Glu Asp Thr Tyr Asn Leu Ala Tyr | |
| 195 200 205 | |
| Val Lys Glu Val Gln Ala Gln Val Leu Val Met Pro Tyr Glu Gly Met | |
| 210 215 220 | |
| Glu Leu Ser Leu Val Val Leu Leu Pro Asp Glu Gly Val Asp Leu Ser | |
| 225 230 235 240 | |
| Lys Val Glu Asn Asn Leu Thr Phe Glu Lys Leu Thr Ala Trp Met Glu | |
| 245 250 255 | |
| Ala Asp Phe Met Lys Ser Thr Asp Val Glu Val Phe Leu Pro Lys Phe | |
| 260 265 270 | |
| Lys Leu Gln Glu Asp Tyr Asp Met Glu Ser Leu Phe Gln Arg Leu Gly | |
| 275 280 285 | |
| Val Val Asp Val Phe Gln Glu Asp Lys Ala Asp Leu Ser Gly Met Ser | |
| 290 295 300 | |
| Pro Glu Arg Asn Leu Cys Val Ser Lys Phe Val His Gln Ser Val Val | |
| 305 310 315 320 | |
| Glu Ile Asn Glu Glu Gly Thr Glu Ala Ala Ala Ala Ser Ala Ile Ile | |
| 325 330 335 | |
| Glu Phe Cys Cys Ala Ser Ser Val Pro Thr Phe Cys Ala Asp His Pro | |
| 340 345 350 | |
| Phe Leu Phe Phe Ile Arg His Asn Lys Ala Asn Ser Ile Leu Phe Cys | |
| 355 360 365 | |
| Gly Arg Phe Ser Ser Pro | |
| 370 | |
| SEQ ID NO: 86 | |
| atgaatactc tgtctgaagg aaatggcacc tttgccatcc atcttttgaa gatgctatgt | 60 |
| caaagcaacc cttccaaaaa tgtatgttat tctcctgcga gcatctcctc tgctctagct | 120 |
| atggttctct tgggtgcaaa gggacagacg gcagtccaga tatctcaggc acttggtttg | 180 |
| aataaagagg aaggcatcca tcagggtttc cagttgcttc tcaggaagct gaacaagcca | 240 |
| gacagaaagt actctcttag agtggccaac aggctctttg cagacaaaac ttgtgaagtc | 300 |
| ctccaaacct ttaaggagtc ctctcttcac ttctatgact cagagatgga gcagctctcc | 360 |
| tttgctgaag aagcagaggt gtccaggcaa cacataaaca catgggtctc caaacaaact | 420 |
| gaaggtaaaa ttccagagtt gttgtcaggt ggctccgtcg attcagaaac caggctggtt | 480 |
| ctcatcaatg ccttatattt taaaggaaag tggcatcaac catttaacaa agagtacaca | 540 |
| atggacatgc cctttaaaat aaacaaggat gagaaaaggc cagtgcagat gatgtgtcgt | 600 |
| gaagacacat ataacctcgc ctatgtgaag gaggtgcagg cgcaagtgct ggtgatgcca | 660 |
| tatgaaggaa tggagctgag cttggtggtt ctgctcccag atgagggtgt ggacctcagc | 720 |
| aaggtggaaa acaatctcac ttttgagaag ttaacagcct ggatggaagc agattttatg | 780 |
| aagagcactg atgttgaggt tttccttcca aaatttaaac tccaagagga ttatgacatg | 840 |
| gagtctctgt ttcagcgctt gggagtggtg gatgtcttcc aagaggacaa ggctgactta | 900 |
| tcaggaatgt ctccagagag aaacctgtgt gtgtccaagt ttgttcacca gagtgtagtg | 960 |
| gagatcaatg aggaaggcag agaggctgca gcagcctctg ccatcataga attttgctgt | 1020 |
| gcctcttctg tcccaacatt ctgtgctgac caccccttcc ttttcttcat caggcacaac | 1080 |
| aaagcaaaca gcatcctgtt ctgtggcagg ttctcatctc cataa | 1125 |
| SEQ ID NO: 87 | |
| Met Asn Thr Leu Ser Glu Gly Asn Gly Thr Phe Ala Ile His Leu Leu | |
| 1 5 10 15 | |
| Lys Met Leu Cys Gln Ser Asn Pro Ser Lys Asn Val Cys Tyr Ser Pro | |
| 20 25 30 | |
| Ala Ser Ile Ser Ser Ala Leu Ala Met Val Leu Leu Gly Ala Lys Gly | |
| 35 40 45 | |
| Gln Thr Ala Val Gln Ile Ser Gln Ala Leu Gly Leu Asn Lys Glu Glu | |
| 50 55 60 | |
| Gly Ile His Gln Gly Phe Gln Leu Leu Leu Arg Lys Leu Asn Lys Pro | |
| 65 70 75 80 | |
| Asp Arg Lys Tyr Ser Leu Arg Val Ala Asn Arg Leu Phe Ala Asp Lys | |
| 85 90 95 | |
| Thr Cys Glu Val Leu Gln Thr Phe Lys Glu Ser Ser Leu His Phe Tyr | |
| 100 105 110 | |
| Asp Ser Glu Met Glu Gln Leu Ser Phe Ala Glu Glu Ala Glu Val Ser | |
| 115 120 125 | |
| Arg Gln His Ile Asn Thr Trp Val Ser Lys Gln Thr Glu Gly Lys Ile | |
| 130 135 140 | |
| Pro Glu Leu Leu Ser Gly Gly Ser Val Asp Ser Glu Thr Arg Leu Val | |
| 145 150 155 160 | |
| Leu Ile Asn Ala Leu Tyr Phe Lys Gly Lys Trp His Gln Pro Phe Asn | |
| 165 170 175 | |
| Lys Glu Tyr Thr Met Asp Met Pro Phe Lys Ile Asn Lys Asp Glu Lys | |
| 180 185 190 | |
| Arg Pro Val Gln Met Met Cys Arg Glu Asp Thr Tyr Asn Leu Ala Tyr | |
| 195 200 205 | |
| Val Lys Glu Val Gln Ala Gln Val Leu Val Met Pro Tyr Glu Gly Met | |
| 210 215 220 | |
| Glu Leu Ser Leu Val Val Leu Leu Pro Asp Glu Gly Val Asp Leu Ser | |
| 225 230 235 240 | |
| Lys Val Glu Asn Asn Leu Thr Phe Glu Lys Leu Thr Ala Trp Met Glu | |
| 245 250 255 | |
| Ala Asp Phe Met Lys Ser Thr Asp Val Glu Val Phe Leu Pro Lys Phe | |
| 260 265 270 | |
| Lys Leu Gln Glu Asp Tyr Asp Met Glu Ser Leu Phe Gln Arg Leu Gly | |
| 275 280 285 | |
| Val Val Asp Val Phe Gln Glu Asp Lys Ala Asp Leu Ser Gly Met Ser | |
| 290 295 300 | |
| Pro Glu Arg Asn Leu Cys Val Ser Lys Phe Val His Gln Ser Val Val | |
| 305 310 315 320 | |
| Glu Ile Asn Glu Glu Gly Arg Glu Ala Ala Ala Ala Ser Ala Ile Ile | |
| 325 330 335 | |
| Glu Phe Cys Cys Ala Ser Ser Val Pro Thr Phe Cys Ala Asp His Pro | |
| 340 345 350 | |
| Phe Leu Phe Phe Ile Arg His Asn Lys Ala Asn Ser Ile Leu Phe Cys | |
| 355 360 365 | |
| Gly Arg Phe Ser Ser Pro | |
| 370 | |
| SEQ ID NO: 88 | |
| gacggatcgg gagatctccc gatcccctat ggtcgactct cagtacaatc tgctctgatg | 60 |
| ccgcatagtt aagccagtat ctgctccctg cttgtgtgtt ggaggtcgct gagtagtgcg | 120 |
| cgagcaaaat ttaagctaca acaaggcaag gcttgaccga caattgcatg aagaatctgc | 180 |
| ttagggttag gcgttttgcg ctgcttcgcg atgtacgggc cagatatacg cgttgacatt | 240 |
| gattattgac tagttattaa tagtaatcaa ttacggggtc attagttcat agcccatata | 300 |
| tggagttccg cgttacataa cttacggtaa atggcccgcc tggctgaccg cccaacgacc | 360 |
| cccgcccatt gacgtcaata atgacgtatg ttcccatagt aacgccaata gggactttcc | 420 |
| attgacgtca atgggtggac tatttacggt aaactgccca cttggcagta catcaagtgt | 480 |
| atcatatgcc aagtacgccc cctattgacg tcaatgacgg taaatggccc gcctggcatt | 540 |
| atgcccagta catgacctta tgggactttc ctacttggca gtacatctac gtattagtca | 600 |
| tcgctattac catggtgatg cggttttggc agtacatcaa tgggcgtgga tagcggtttg | 660 |
| actcacgggg atttccaagt ctccacccca ttgacgtcaa tgggagtttg ttttggcacc | 720 |
| aaaatcaacg ggactttcca aaatgtcgta acaactccgc cccattgacg caaatgggcg | 780 |
| gtaggcgtgt acggtgggag gtctatataa gcagagctct ctggctaact agagaaccca | 840 |
| ctgcttactg gcttatcgaa attaatacga ctcactatag ggagacccaa gctggctagc | 900 |
| gtttaaacgg gccctctaga ctcgagcggc cgccactgtg ctggatatct gcagaattca | 960 |
| tgaatactct gtctgaagga aatggcacct ttgccatcca tcttttgaag atgctatgtc | 1020 |
| aaagcaaccc ttccaaaaat gtatgttatt ctcctgcgag catctcctct gctctagcta | 1080 |
| tggttctctt gggtgcaaag ggacagacgg cagtccagat atctcaggca cttggtttga | 1140 |
| ataaagagga aggcatccat cagggtttcc agttgcttct caggaagctg aacaagccag | 1200 |
| acagaaagta ctctcttaga gtggccaaca ggctctttgc agacaaaact tgtgaagtcc | 1260 |
| tccaaacctt taaggagtcc tctcttcact tctatgactc agagatggag cagctctcct | 1320 |
| ttgctgaaga agcagaggtg tccaggcaac acataaacac atgggtctcc aaacaaactg | 1380 |
| aaggtaaaat tccagagttg ttgtcaggtg gctccgtcga ttcagaaacc aggctggttc | 1440 |
| tcatcaatgc cttatatttt aaaggaaagt ggcatcaacc atttaacaaa gagtacacaa | 1500 |
| tggacatgcc ctttaaaata aacaaggatg agaaaaggcc agtgcagatg atgtgtcgtg | 1560 |
| aagacacata taacctcgcc tatgtgaagg aggtgcaggc gcaagtgctg gtgatgccat | 1620 |
| atgaaggaat ggagctgagc ttggtggttc tgctcccaga tgagggtgtg gacctcagca | 1680 |
| aggtggaaaa caatctcact tttgagaagt taacagcctg gatggaagca gattttatga | 1740 |
| agagcactga tgttgaggtt ttccttccaa aatttaaact ccaagaggat tatgacatgg | 1800 |
| agtctctgtt tcagcgcttg ggagtggtgg atgtcttcca agaggacaag gctgacttat | 1860 |
| caggaatgtc tccagagaga aacctgtgtg tgtccaagtt tgttcaccag agtgtagtgg | 1920 |
| agatcaatga ggaaggcaca gaggctgcag cagcctctgc catcatagaa ttttgctgtg | 1980 |
| cctcttctgt cccaacattc tgtgctgacc accccttcct tttcttcatc aggcacaaca | 2040 |
| aagcaaacag catcctgttc tgtggcaggt tctcatctcc ataaggatcc gagctcggta | 2100 |
| ccaagcttaa gtttaaaccg ctgatcagcc tcgactgtgc cttctagttg ccagccatct | 2160 |
| gttgtttgcc cctcccccgt gccttccttg accctggaag gtgccactcc cactgtcctt | 2220 |
| tcctaataaa atgaggaaat tgcatcgcat tgtctgagta ggtgtcattc tattctgggg | 2280 |
| ggtggggtgg ggcaggacag caagggggag gattgggaag acaatagcag gcatgctggg | 2340 |
| gatgcggtgg gctctatggc ttctgaggcg gaaagaacca gctggggctc tagggggtat | 2400 |
| ccccacgcgc cctgtagcgg cgcattaagc gcggcgggtg tggtggttac gcgcagcgtg | 2460 |
| accgctacac ttgccagcgc cctagcgccc gctcctttcg ctttcttccc ttcctttctc | 2520 |
| gccacgttcg ccggctttcc ccgtcaagct ctaaatcggg gcatcccttt agggttccga | 2580 |
| tttagtgctt tacggcacct cgaccccaaa aaacttgatt agggtgatgg ttcacgtagt | 2640 |
| gggccatcgc cctgatagac ggtttttcgc cctttgacgt tggagtccac gttctttaat | 2700 |
| agtggactct tgttccaaac tggaacaaca ctcaacccta tctcggtcta ttcttttgat | 2760 |
| ttataaggga ttttggggat ttcggcctat tggttaaaaa atgagctgat ttaacaaaaa | 2820 |
| tttaacgcga attaattctg tggaatgtgt gtcagttagg gtgtggaaag tccccaggct | 2880 |
| ccccaggcag gcagaagtat gcaaagcatg catctcaatt agtcagcaac caggtgtgga | 2940 |
| aagtccccag gctccccagc aggcagaagt atgcaaagca tgcatctcaa ttagtcagca | 3000 |
| accatagtcc cgcccctaac tccgcccatc ccgcccctaa ctccgcccag ttccgcccat | 3060 |
| tctccgcccc atggctgact aatttttttt atttatgcag aggccgaggc cgcctctgcc | 3120 |
| tctgagctat tccagaagta gtgaggaggc ttttttggag gcctaggctt ttgcaaaaag | 3180 |
| ctcccgggag cttgtatatc cattttcgga tctgatcaag agacaggatg aggatcgttt | 3240 |
| cgcatgattg aacaagatgg attgcacgca ggttctccgg ccgcttgggt ggagaggcta | 3300 |
| ttcggctatg actgggcaca acagacaatc ggctgctctg atgccgccgt gttccggctg | 3360 |
| tcagcgcagg ggcgcccggt tctttttgtc aagaccgacc tgtccggtgc cctgaatgaa | 3420 |
| ctgcaggacg aggcagcgcg gctatcgtgg ctggccacga cgggcgttcc ttgcgcagct | 3480 |
| gtgctcgacg ttgtcactga agcgggaagg gactggctgc tattgggcga agtgccgggg | 3540 |
| caggatctcc tgtcatctca ccttgctcct gccgagaaag tatccatcat ggctgatgca | 3600 |
| atgcggcggc tgcatacgct tgatccggct acctgcccat tcgaccacca agcgaaacat | 3660 |
| cgcatcgagc gagcacgtac tcggatggaa gccggtcttg tcgatcagga tgatctggac | 3720 |
| gaagagcatc aggggctcgc gccagccgaa ctgttcgcca ggctcaaggc gcgcatgccc | 3780 |
| gacggcgagg atctcgtcgt gacccatggc gatgcctgct tgccgaatat catggtggaa | 3840 |
| aatggccgct tttctggatt catcgactgt ggccggctgg gtgtggcgga ccgctatcag | 3900 |
| gacatagcgt tggctacccg tgatattgct gaagagcttg gcggcgaatg ggctgaccgc | 3960 |
| ttcctcgtgc tttacggtat cgccgctccc gattcgcagc gcatcgcctt ctatcgcctt | 4020 |
| cttgacgagt tcttctgagc gggactctgg ggttcgaaat gaccgaccaa gcgacgccca | 4080 |
| acctgccatc acgagatttc gattccaccg ccgccttcta tgaaaggttg ggcttcggaa | 4140 |
| tcgttttccg ggacgccggc tggatgatcc tccagcgcgg ggatctcatg ctggagttct | 4200 |
| tcgcccaccc caacttgttt attgcagctt ataatggtta caaataaagc aatagcatca | 4260 |
| caaatttcac aaataaagca tttttttcac tgcattctag ttgtggtttg tccaaactca | 4320 |
| tcaatgtatc ttatcatgtc tgtataccgt cgacctctag ctagagcttg gcgtaatcat | 4380 |
| ggtcatagct gtttcctgtg tgaaattgtt atccgctcac aattccacac aacatacgag | 4440 |
| ccggaagcat aaagtgtaaa gcctggggtg cctaatgagt gagctaactc acattaattg | 4500 |
| cgttgcgctc actgcccgct ttccagtcgg gaaacctgtc gtgccagctg cattaatgaa | 4560 |
| tcggccaacg cgcggggaga ggcggtttgc gtattgggcg ctcttccgct tcctcgctca | 4620 |
| ctgactcgct gcgctcggtc gttcggctgc ggcgagcggt atcagctcac tcaaaggcgg | 4680 |
| taatacggtt atccacagaa tcaggggata acgcaggaaa gaacatgtga gcaaaaggcc | 4740 |
| agcaaaaggc caggaaccgt aaaaaggccg cgttgctggc gtttttccat aggctccgcc | 4800 |
| cccctgacga gcatcacaaa aatcgacgct caagtcagag gtggcgaaac ccgacaggac | 4860 |
| tataaagata ccaggcgttt ccccctggaa gctccctcgt gcgctctcct gttccgaccc | 4920 |
| tgccgcttac cggatacctg tccgcctttc tcccttcggg aagcgtggcg ctttctcaat | 4980 |
| gctcacgctg taggtatctc agttcggtgt aggtcgttcg ctccaagctg ggctgtgtgc | 5040 |
| acgaaccccc cgttcagccc gaccgctgcg ccttatccgg taactatcgt cttgagtcca | 5100 |
| acccggtaag acacgactta tcgccactgg cagcagccac tggtaacagg attagcagag | 5160 |
| cgaggtatgt aggcggtgct acagagttct tgaagtggtg gcctaactac ggctacacta | 5220 |
| gaaggacagt atttggtatc tgcgctctgc tgaagccagt taccttcgga aaaagagttg | 5280 |
| gtagctcttg atccggcaaa caaaccaccg ctggtagcgg tggttttttt gtttgcaagc | 5340 |
| agcagattac gcgcagaaaa aaaggatctc aagaagatcc tttgatcttt tctacggggt | 5400 |
| ctgacgctca gtggaacgaa aactcacgtt aagggatttt ggtcatgaga ttatcaaaaa | 5460 |
| ggatcttcac ctagatcctt ttaaattaaa aatgaagttt taaatcaatc taaagtatat | 5520 |
| atgagtaaac ttggtctgac agttaccaat gcttaatcag tgaggcacct atctcagcga | 5580 |
| tctgtctatt tcgttcatcc atagttgcct gactccccgt cgtgtagata actacgatac | 5640 |
| gggagggctt accatctggc cccagtgctg caatgatacc gcgagaccca cgctcaccgg | 5700 |
| ctccagattt atcagcaata aaccagccag ccggaagggc cgagcgcaga agtggtcctg | 5760 |
| caactttatc cgcctccatc cagtctatta attgttgccg ggaagctaga gtaagtagtt | 5820 |
| cgccagttaa tagtttgcgc aacgttgttg ccattgctac aggcatcgtg gtgtcacgct | 5880 |
| cgtcgtttgg tatggcttca ttcagctccg gttcccaacg atcaaggcga gttacatgat | 5940 |
| cccccatgtt gtgcaaaaaa gcggttagct ccttcggtcc tccgatcgtt gtcagaagta | 6000 |
| agttggccgc agtgttatca ctcatggtta tggcagcact gcataattct cttactgtca | 6060 |
| tgccatccgt aagatgcttt tctgtgactg gtgagtactc aaccaagtca ttctgagaat | 6120 |
| agtgtatgcg gcgaccgagt tgctcttgcc cggcgtcaat acgggataat accgcgccac | 6180 |
| atagcagaac tttaaaagtg ctcatcattg gaaaacgttc ttcggggcga aaactctcaa | 6240 |
| ggatcttacc gctgttgaga tccagttcga tgtaacccac tcgtgcaccc aactgatctt | 6300 |
| cagcatcttt tactttcacc agcgtttctg ggtgagcaaa aacaggaagg caaaatgccg | 6360 |
| caaaaaaggg aataagggcg acacggaaat gttgaatact catactcttc ctttttcaat | 6420 |
| attattgaag catttatcag ggttattgtc tcatgagcgg atacatattt gaatgtattt | 6480 |
| agaaaaataa acaaataggg gttccgcgca catttccccg aaaagtgcca cctgacgtc | 6539 |
| SEQ ID NO: 89 | |
| gacggatcgg gagatctccc gatcccctat ggtcgactct cagtacaatc tgctctgatg | 60 |
| ccgcatagtt aagccagtat ctgctccctg cttgtgtgtt ggaggtcgct gagtagtgcg | 120 |
| cgagcaaaat ttaagctaca acaaggcaag gcttgaccga caattgcatg aagaatctgc | 180 |
| ttagggttag gcgttttgcg ctgcttcgcg atgtacgggc cagatatacg cgttgacatt | 240 |
| gattattgac tagttattaa tagtaatcaa ttacggggtc attagttcat agcccatata | 300 |
| tggagttccg cgttacataa cttacggtaa atggcccgcc tggctgaccg cccaacgacc | 360 |
| cccgcccatt gacgtcaata atgacgtatg ttcccatagt aacgccaata gggactttcc | 420 |
| attgacgtca atgggtggac tatttacggt aaactgccca cttggcagta catcaagtgt | 480 |
| atcatatgcc aagtacgccc cctattgacg tcaatgacgg taaatggccc gcctggcatt | 540 |
| atgcccagta catgacctta tgggactttc ctacttggca gtacatctac gtattagtca | 600 |
| tcgctattac catggtgatg cggttttggc agtacatcaa tgggcgtgga tagcggtttg | 660 |
| actcacgggg atttccaagt ctccacccca ttgacgtcaa tgggagtttg ttttggcacc | 720 |
| aaaatcaacg ggactttcca aaatgtcgta acaactccgc cccattgacg caaatgggcg | 780 |
| gtaggcgtgt acggtgggag gtctatataa gcagagctct ctggctaact agagaaccca | 840 |
| ctgcttactg gcttatcgaa attaatacga ctcactatag ggagacccaa gctggctagc | 900 |
| gtttaaacgg gccctctaga ctcgagcggc cgccactgtg ctggatatct gcagaattca | 960 |
| tgaatactct gtctgaagga aatggcacct ttgccatcca tcttttgaag atgctatgtc | 1020 |
| aaagcaaccc ttccaaaaat gtatgttatt ctcctgcgag catctcctct gctctagcta | 1080 |
| tggttctctt gggtgcaaag ggacagacgg cagtccagat atctcaggca cttggtttga | 1140 |
| ataaagagga aggcatccat cagggtttcc agttgcttct caggaagctg aacaagccag | 1200 |
| acagaaagta ctctcttaga gtggccaaca ggctctttgc agacaaaact tgtgaagtcc | 1260 |
| tccaaacctt taaggagtcc tctcttcact tctatgactc agagatggag cagctctcct | 1320 |
| ttgctgaaga agcagaggtg tccaggcaac acataaacac atgggtctcc aaacaaactg | 1380 |
| aaggtaaaat tccagagttg ttgtcaggtg gctccgtcga ttcagaaacc aggctggttc | 1440 |
| tcatcaatgc cttatatttt aaaggaaagt ggcatcaacc atttaacaaa gagtacacaa | 1500 |
| tggacatgcc ctttaaaata aacaaggatg agaaaaggcc agtgcagatg atgtgtcgtg | 1560 |
| aagacacata taacctcgcc tatgtgaagg aggtgcaggc gcaagtgctg gtgatgccat | 1620 |
| atgaaggaat ggagctgagc ttggtggttc tgctcccaga tgagggtgtg gacctcagca | 1680 |
| aggtggaaaa caatctcact tttgagaagt taacagcctg gatggaagca gattttatga | 1740 |
| agagcactga tgttgaggtt ttccttccaa aatttaaact ccaagaggat tatgacatgg | 1800 |
| agtctctgtt tcagcgcttg ggagtggtgg atgtcttcca agaggacaag gctgacttat | 1860 |
| caggaatgtc tccagagaga aacctgtgtg tgtccaagtt tgttcaccag agtgtagtgg | 1920 |
| agatcaatga ggaaggcaga gaggctgcag cagcctctgc catcatagaa ttttgctgtg | 1980 |
| cctcttctgt cccaacattc tgtgctgacc accccttcct tttcttcatc aggcacaaca | 2040 |
| aagcaaacag catcctgttc tgtggcaggt tctcatctcc ataaggatcc gagctcggta | 2100 |
| ccaagcttaa gtttaaaccg ctgatcagcc tcgactgtgc cttctagttg ccagccatct | 2160 |
| gttgtttgcc cctcccccgt gccttccttg accctggaag gtgccactcc cactgtcctt | 2220 |
| tcctaataaa atgaggaaat tgcatcgcat tgtctgagta ggtgtcattc tattctgggg | 2280 |
| ggtggggtgg ggcaggacag caagggggag gattgggaag acaatagcag gcatgctggg | 2340 |
| gatgcggtgg gctctatggc ttctgaggcg gaaagaacca gctggggctc tagggggtat | 2400 |
| ccccacgcgc cctgtagcgg cgcattaagc gcggcgggtg tggtggttac gcgcagcgtg | 2460 |
| accgctacac ttgccagcgc cctagcgccc gctcctttcg ctttcttccc ttcctttctc | 2520 |
| gccacgttcg ccggctttcc ccgtcaagct ctaaatcggg gcatcccttt agggttccga | 2580 |
| tttagtgctt tacggcacct cgaccccaaa aaacttgatt agggtgatgg ttcacgtagt | 2640 |
| gggccatcgc cctgatagac ggtttttcgc cctttgacgt tggagtccac gttctttaat | 2700 |
| agtggactct tgttccaaac tggaacaaca ctcaacccta tctcggtcta ttcttttgat | 2760 |
| ttataaggga ttttggggat ttcggcctat tggttaaaaa atgagctgat ttaacaaaaa | 2820 |
| tttaacgcga attaattctg tggaatgtgt gtcagttagg gtgtggaaag tccccaggct | 2880 |
| ccccaggcag gcagaagtat gcaaagcatg catctcaatt agtcagcaac caggtgtgga | 2940 |
| aagtccccag gctccccagc aggcagaagt atgcaaagca tgcatctcaa ttagtcagca | 3000 |
| accatagtcc cgcccctaac tccgcccatc ccgcccctaa ctccgcccag ttccgcccat | 3060 |
| tctccgcccc atggctgact aatttttttt atttatgcag aggccgaggc cgcctctgcc | 3120 |
| tctgagctat tccagaagta gtgaggaggc ttttttggag gcctaggctt ttgcaaaaag | 3180 |
| ctcccgggag cttgtatatc cattttcgga tctgatcaag agacaggatg aggatcgttt | 3240 |
| cgcatgattg aacaagatgg attgcacgca ggttctccgg ccgcttgggt ggagaggcta | 3300 |
| ttcggctatg actgggcaca acagacaatc ggctgctctg atgccgccgt gttccggctg | 3360 |
| tcagcgcagg ggcgcccggt tctttttgtc aagaccgacc tgtccggtgc cctgaatgaa | 3420 |
| ctgcaggacg aggcagcgcg gctatcgtgg ctggccacga cgggcgttcc ttgcgcagct | 3480 |
| gtgctcgacg ttgtcactga agcgggaagg gactggctgc tattgggcga agtgccgggg | 3540 |
| caggatctcc tgtcatctca ccttgctcct gccgagaaag tatccatcat ggctgatgca | 3600 |
| atgcggcggc tgcatacgct tgatccggct acctgcccat tcgaccacca agcgaaacat | 3660 |
| cgcatcgagc gagcacgtac tcggatggaa gccggtcttg tcgatcagga tgatctggac | 3720 |
| gaagagcatc aggggctcgc gccagccgaa ctgttcgcca ggctcaaggc gcgcatgccc | 3780 |
| gacggcgagg atctcgtcgt gacccatggc gatgcctgct tgccgaatat catggtggaa | 3840 |
| aatggccgct tttctggatt catcgactgt ggccggctgg gtgtggcgga ccgctatcag | 3900 |
| gacatagcgt tggctacccg tgatattgct gaagagcttg gcggcgaatg ggctgaccgc | 3960 |
| ttcctcgtgc tttacggtat cgccgctccc gattcgcagc gcatcgcctt ctatcgcctt | 4020 |
| cttgacgagt tcttctgagc gggactctgg ggttcgaaat gaccgaccaa gcgacgccca | 4080 |
| acctgccatc acgagatttc gattccaccg ccgccttcta tgaaaggttg ggcttcggaa | 4140 |
| tcgttttccg ggacgccggc tggatgatcc tccagcgcgg ggatctcatg ctggagttct | 4200 |
| tcgcccaccc caacttgttt attgcagctt ataatggtta caaataaagc aatagcatca | 4260 |
| caaatttcac aaataaagca tttttttcac tgcattctag ttgtggtttg tccaaactca | 4320 |
| tcaatgtatc ttatcatgtc tgtataccgt cgacctctag ctagagcttg gcgtaatcat | 4380 |
| ggtcatagct gtttcctgtg tgaaattgtt atccgctcac aattccacac aacatacgag | 4440 |
| ccggaagcat aaagtgtaaa gcctggggtg cctaatgagt gagctaactc acattaattg | 4500 |
| cgttgcgctc actgcccgct ttccagtcgg gaaacctgtc gtgccagctg cattaatgaa | 4560 |
| tcggccaacg cgcggggaga ggcggtttgc gtattgggcg ctcttccgct tcctcgctca | 4620 |
| ctgactcgct gcgctcggtc gttcggctgc ggcgagcggt atcagctcac tcaaaggcgg | 4680 |
| taatacggtt atccacagaa tcaggggata acgcaggaaa gaacatgtga gcaaaaggcc | 4740 |
| agcaaaaggc caggaaccgt aaaaaggccg cgttgctggc gtttttccat aggctccgcc | 4800 |
| cccctgacga gcatcacaaa aatcgacgct caagtcagag gtggcgaaac ccgacaggac | 4860 |
| tataaagata ccaggcgttt ccccctggaa gctccctcgt gcgctctcct gttccgaccc | 4920 |
| tgccgcttac cggatacctg tccgcctttc tcccttcggg aagcgtggcg ctttctcaat | 4980 |
| gctcacgctg taggtatctc agttcggtgt aggtcgttcg ctccaagctg ggctgtgtgc | 5040 |
| acgaaccccc cgttcagccc gaccgctgcg ccttatccgg taactatcgt cttgagtcca | 5100 |
| acccggtaag acacgactta tcgccactgg cagcagccac tggtaacagg attagcagag | 5160 |
| cgaggtatgt aggcggtgct acagagttct tgaagtggtg gcctaactac ggctacacta | 5220 |
| gaaggacagt atttggtatc tgcgctctgc tgaagccagt taccttcgga aaaagagttg | 5280 |
| gtagctcttg atccggcaaa caaaccaccg ctggtagcgg tggttttttt gtttgcaagc | 5340 |
| agcagattac gcgcagaaaa aaaggatctc aagaagatcc tttgatcttt tctacggggt | 5400 |
| ctgacgctca gtggaacgaa aactcacgtt aagggatttt ggtcatgaga ttatcaaaaa | 5460 |
| ggatcttcac ctagatcctt ttaaattaaa aatgaagttt taaatcaatc taaagtatat | 5520 |
| atgagtaaac ttggtctgac agttaccaat gcttaatcag tgaggcacct atctcagcga | 5580 |
| tctgtctatt tcgttcatcc atagttgcct gactccccgt cgtgtagata actacgatac | 5640 |
| gggagggctt accatctggc cccagtgctg caatgatacc gcgagaccca cgctcaccgg | 5700 |
| ctccagattt atcagcaata aaccagccag ccggaagggc cgagcgcaga agtggtcctg | 5760 |
| caactttatc cgcctccatc cagtctatta attgttgccg ggaagctaga gtaagtagtt | 5820 |
| cgccagttaa tagtttgcgc aacgttgttg ccattgctac aggcatcgtg gtgtcacgct | 5880 |
| cgtcgtttgg tatggcttca ttcagctccg gttcccaacg atcaaggcga gttacatgat | 5940 |
| cccccatgtt gtgcaaaaaa gcggttagct ccttcggtcc tccgatcgtt gtcagaagta | 6000 |
| agttggccgc agtgttatca ctcatggtta tggcagcact gcataattct cttactgtca | 6060 |
| tgccatccgt aagatgcttt tctgtgactg gtgagtactc aaccaagtca ttctgagaat | 6120 |
| agtgtatgcg gcgaccgagt tgctcttgcc cggcgtcaat acgggataat accgcgccac | 6180 |
| atagcagaac tttaaaagtg ctcatcattg gaaaacgttc ttcggggcga aaactctcaa | 6240 |
| ggatcttacc gctgttgaga tccagttcga tgtaacccac tcgtgcaccc aactgatctt | 6300 |
| cagcatcttt tactttcacc agcgtttctg ggtgagcaaa aacaggaagg caaaatgccg | 6360 |
| caaaaaaggg aataagggcg acacggaaat gttgaatact catactcttc ctttttcaat | 6420 |
| attattgaag catttatcag ggttattgtc tcatgagcgg atacatattt gaatgtattt | 6480 |
| agaaaaataa acaaataggg gttccgcgca catttccccg aaaagtgcca cctgacgtc | 6539 |
| SEQ ID NO: 90 | |
| atggatgacc agcgcgacct tatctccaac aatgagcaac tgcccatgct gggccggcgc | 60 |
| cctggggccc cggagagcaa gtgcagccgc ggagccctgt acacaggctt ttccatcctg | 120 |
| gtgactctgc tcctcgctgg ccaggccacc accgcctact tcctgtacca gcagcagggc | 180 |
| cggctggaca aactgacagt cacctcccag aacctgcagc tggagaacct gcgcatgaag | 240 |
| cttgccaagt tcgtggctgc ctggaccctg aaggctgccg ctgccctgcc ccaggggccc | 300 |
| atgcagaatg ccaccaagta tggcaacatg acagaggacc atgtgatgca cctgctccag | 360 |
| aatgctgacc ccctgaaggt gtacccgcca ctgaagggga gcttcccgga gaacctgaga | 420 |
| caccttaaga acaccatgga gaccatagac tggaaggtct ttgagagctg gatgcaccat | 480 |
| tggctcctgt ttgaaatgag caggcactcc ttggagcaaa agcccactga cgctccaccg | 540 |
| aaagtactga ccaagtgcca ggaagaggtc agccacatcc ctgctgtcca cccgggttca | 600 |
| ttcaggccca agtgcgacga gaacggcaac tatctgccac tccagtgcta tgggagcatc | 660 |
| ggctactgct ggtgtgtctt ccccaacggc acggaggtcc ccaacaccag aagccgcggg | 720 |
| caccataact gcagtgagtc actggaactg gaggacccgt cttctgggct gggtgtgacc | 780 |
| aagcaggatc tgggcccagt ccccatgtga | 810 |
| SEQ ID NO: 91 | |
| Met Asp Asp Gln Arg Asp Leu Ile Ser Asn Asn Glu Gln Leu Pro Met | |
| 1 5 10 15 | |
| Leu Gly Arg Arg Pro Gly Ala Pro Glu Ser Lys Cys Ser Arg Gly Ala | |
| 20 25 30 | |
| Leu Tyr Thr Gly Phe Ser Ile Leu Val Thr Leu Leu Leu Ala Gly Gln | |
| 35 40 45 | |
| Ala Thr Thr Ala Tyr Phe Leu Tyr Gln Gln Gln Gly Arg Leu Asp Lys | |
| 50 55 60 | |
| Leu Thr Val Thr Ser Gln Asn Leu Gln Leu Glu Asn Leu Arg Met Lys | |
| 65 70 75 80 | |
| Leu Ala Lys Phe Val Ala Ala Trp Thr Leu Lys Ala Ala Ala Ala Leu | |
| 85 90 95 | |
| Pro Gln Gly Pro Met Gln Asn Ala Thr Lys Tyr Gly Asn Met Thr Glu | |
| 100 105 110 | |
| Asp His Val Met His Leu Leu Gln Asn Ala Asp Pro Leu Lys Val Tyr | |
| 115 120 125 | |
| Pro Pro Leu Lys Gly Ser Phe Pro Glu Asn Leu Arg His Leu Lys Asn | |
| 130 135 140 | |
| Thr Met Glu Thr Ile Asp Trp Lys Val Phe Glu Ser Trp Met His His | |
| 145 150 155 160 | |
| Trp Leu Leu Phe Glu Met Ser Arg His Ser Leu Glu Gln Lys Pro Thr | |
| 165 170 175 | |
| Asp Ala Pro Pro Lys Val Leu Thr Lys Cys Gln Glu Glu Val Ser His | |
| 180 185 190 | |
| Ile Pro Ala Val His Pro Gly Ser Phe Arg Pro Lys Cys Asp Glu Asn | |
| 195 200 205 | |
| Gly Asn Tyr Leu Pro Leu Gln Cys Tyr Gly Ser Ile Gly Tyr Cys Trp | |
| 210 215 220 | |
| Cys Val Phe Pro Asn Gly Thr Glu Val Pro Asn Thr Arg Ser Arg Gly | |
| 225 230 235 240 | |
| His His Asn Cys Ser Glu Ser Leu Glu Leu Glu Asp Pro Ser Ser Gly | |
| 245 250 255 | |
| Leu Gly Val Thr Lys Gln Asp Leu Gly Pro Val Pro Met | |
| 260 265 | |
| SEQ ID NO: 92 | |
| Lys Pro Val Ser Gln Met Arg Met Ala Thr Pro Leu Leu Met Arg Pro | |
| 1 5 10 15 | |
| Met | |
| SEQ ID NO: 93 | |
| Ala Lys Phe Val Ala Ala Trp Thr Leu Lys Ala Ala Ala | |
| 1 5 10 | |
| SEQ ID NO: 94 | |
| atgcgttgcc tggctccacg ccctgctggg tcctacctgt cagagcccca aggcagctca | 60 |
| cagtgtgcca ccatggagtt ggggccccta gaaggtggct acctggagct tcttaacagc | 120 |
| gatgctgacc cctgtgcctc taccacttct atgaccagat ggacctggct ggagaagaag | 180 |
| agattgagct ctactcagaa cccgacacag acaccatcaa ctgcgaccag ttcagcaggc | 240 |
| tgttgtgtga catggaaggt gatgaagaga ccagggaggc ttatgccaat atcgcggaac | 300 |
| tggaccagta tgtcttccag gactcccagc tggagggcct gagcaaggac attttcaagc | 360 |
| acataggacc agatgaagtg atcggtgaga gtatggagat gccagcagaa gttgggcaga | 420 |
| aaagtcagaa aagacccttc ccagaggagc ttccggcaga cctgaagcac tggaagccag | 480 |
| ctgagccccc cactgtggtg actggcagtc tcctagtggg accagtgagc gactgctcca | 540 |
| ccctgccctg cctgccactg cctgcgctgt tcaaccagga gccagcctcc ggccagatgc | 600 |
| gcctggagaa aaccgaccag attcccatgc ctttctccag ttcctcgttg agctgcctga | 660 |
| atctccctga gggacccatc cagtttgtcc ccaccatctc cactctgccc catgggctct | 720 |
| ggcaaatctc tgaggctgga acaggggtct ccagtatatt catctaccat ggtgaggtgc | 780 |
| cccaggccag ccaagtaccc cctcccagtg gattcactgt ccacggcctc ccaacatctc | 840 |
| cagaccggcc aggctccacc agccccttcg ctccatcagc cactgacctg cccagcatgc | 900 |
| ctgaacctgc cctgacctcc cgagcaaaca tgacagagca caagacgtcc cccacccaat | 960 |
| gcccggcagc tggagaggtc tccaacaagc ttccaaaatg gcctgagccg gtggagcagt | 1020 |
| tctaccgctc actgcaggac acgtatggtg ccgagcccgc aggcccggat ggcatcctag | 1080 |
| tggaggtgga tctggtgcag gccaggctgg agaggagcag cagcaagagc ctggagcggg | 1140 |
| aactggccac cccggactgg gcagaacggc agctggccca aggaggcctg gctgaggtgc | 1200 |
| tgttggctgc caaggagcac cggcggccgc gtgagacacg agtgattgct gtgctgggca | 1260 |
| aagctggtca gggcaagagc tattgggctg gggcagtgag ccgggcctgg gcttgtggcc | 1320 |
| ggcttcccca gtacgacttt gtcttctctg tcccctgcca ttgcttgaac cgtccggggg | 1380 |
| atgcctatgg cctgcaggat ctgctcttct ccctgggccc acagccactc gtggcggccg | 1440 |
| atgaggtttt cagccacatc ttgaagagac ctgaccgcgt tctgctcatc ctagacggct | 1500 |
| tcgaggagct ggaagcgcaa gatggcttcc tgcacagcac gtgcggaccg gcaccggcgg | 1560 |
| agccctgctc cctccggggg ctgctggccg gccttttcca gaagaagctg ctccgaggtt | 1620 |
| gcaccctcct cctcacagcc cggccccggg gccgcctggt ccagagcctg agcaaggccg | 1680 |
| acgccctatt tgagctgtcc ggcttctcca tggagcaggc ccaggcatac gtgatgcgct | 1740 |
| actttgagag ctcagggatg acagagcacc aagacagagc cctgacgctc ctccgggacc | 1800 |
| ggccacttct tctcagtcac agccacagcc ctactttgtg ccgggcagtg tgccagctct | 1860 |
| cagaggccct gctggagctt ggggaggacg ccaagctgcc ctccacgctc acgggactct | 1920 |
| atgtcggcct gctgggccgt gcagccctcg acagcccccc cggggccctg gcagagctgg | 1980 |
| ccaagctggc ctgggagctg ggccgcagac atcaaagtac cctacaggag gaccagttcc | 2040 |
| catccgcaga cgtgaggacc tgggcgatgg ccaaaggctt agtccaacac ccaccgcggg | 2100 |
| ccgcagagtc cgagctggcc ttccccagct tcctcctgca atgcttcctg ggggccctgt | 2160 |
| ggctggctct gagtggcgaa atcaaggaca aggagctccc gcagtaccta gcattgaccc | 2220 |
| caaggaagaa gaggccctat gacaactggc tggagggcgt gccacgcttt ctggctgggc | 2280 |
| tgatcttcca gcctcccgcc cgctgcctgg gagccatact cgggccatcg gcggctgcct | 2340 |
| cggtggacag gaagcagaag gtgcttgcga ggtacctgaa gcggctgcag ccggggacac | 2400 |
| tgcgggcgcg gcagctgctg gagctgctgc actgcgccca cgaggccgag gaggctggaa | 2460 |
| tttggcagca cgtggtacag gagctccccg gccgcctctc ttttctgggc acccgcctca | 2520 |
| cgcctcctga tgcacatgta ctgggcaagg ccttggaggc ggcgggccaa gacttctccc | 2580 |
| tggacctccg cagcactggc atttgcccct ctggattggg gagcctcgtg ggactcagct | 2640 |
| gtgtcacccg tttcagggct gccttgagcg acacggtggc gctgtgggag tccctgcagc | 2700 |
| agcatgggga gaccaagcta cttcaggcag cagaggagaa gttcaccatc gagcctttca | 2760 |
| aagccaagtc cctgaaggat gtggaagacc tgggaaagct tgtgcagact cagaggacga | 2820 |
| gaagttcctc ggaagacaca gctggggagc tccctgctgt tcgggaccta aagaaactgg | 2880 |
| agtttgcgct gggccctgtc tcaggccccc aggctttccc caaactggtg cggatcctca | 2940 |
| cggccttttc ctccctgcag catctggacc tggatgcgct gagtgagaac aagatcgggg | 3000 |
| acgagggtgt ctcgcagctc tcagccacct tcccccagct gaagtccttg gaaaccctca | 3060 |
| atctgtccca gaacaacatc actgacctgg gtgcctacaa actcgccgag gccctgcctt | 3120 |
| cgctcgctgc atccctgctc aggctaagct tgtacaataa ctgcatctgc gacgtgggag | 3180 |
| ccgagagctt ggctcgtgtg cttccggaca tggtgtccct ccgggtgatg gacgtccagt | 3240 |
| acaacaagtt cacggctgcc ggggcccagc agctcgctgc cagccttcgg aggtgtcctc | 3300 |
| atgtggagac gctggcgatg tggacgccca ccatcccatt cagtgtccag gaacacctgc | 3360 |
| aacaacagga ttcacggatc agcctgagat ga | 3392 |
| SEQ ID NO: 95 | |
| Met Arg Cys Leu Ala Pro Arg Pro Ala Gly Ser Tyr Leu Ser Glu Pro | |
| 1 5 10 15 | |
| Gln Gly Ser Ser Gln Cys Ala Thr Met Glu Leu Gly Pro Leu Glu Gly | |
| 20 25 30 | |
| Gly Tyr Leu Glu Leu Leu Asn Ser Asp Ala Asp Pro Leu Cys Leu Tyr | |
| 35 40 45 | |
| His Phe Tyr Asp Gln Met Asp Leu Ala Gly Glu Glu Glu Ile Glu Leu | |
| 50 55 60 | |
| Tyr Ser Glu Pro Asp Thr Asp Thr Ile Asn Cys Asp Gln Phe Ser Arg | |
| 65 70 75 80 | |
| Leu Leu Cys Asp Met Glu Gly Asp Glu Glu Thr Arg Glu Ala Tyr Ala | |
| 85 90 95 | |
| Asn Ile Ala Glu Leu Asp Gln Tyr Val Phe Gln Asp Ser Gln Leu Glu | |
| 100 105 110 | |
| Gly Leu Ser Lys Asp Ile Phe Lys His Ile Gly Pro Asp Glu Val Ile | |
| 115 120 125 | |
| Gly Glu Ser Met Glu Met Pro Ala Glu Val Gly Gln Lys Ser Gln Lys | |
| 130 135 140 | |
| Arg Pro Phe Pro Glu Glu Leu Pro Ala Asp Leu Lys His Trp Lys Pro | |
| 145 150 155 160 | |
| Ala Glu Pro Pro Thr Val Val Thr Gly Ser Leu Leu Val Gly Pro Val | |
| 165 170 175 | |
| Ser Asp Cys Ser Thr Leu Pro Cys Leu Pro Leu Pro Ala Leu Phe Asn | |
| 180 185 190 | |
| Gln Glu Pro Ala Ser Gly Gln Met Arg Leu Glu Lys Thr Asp Gln Ile | |
| 195 200 205 | |
| Pro Met Pro Phe Ser Ser Ser Ser Leu Ser Cys Leu Asn Leu Pro Glu | |
| 210 215 220 | |
| Gly Pro Ile Gln Phe Val Pro Thr Ile Ser Thr Leu Pro His Gly Leu | |
| 225 230 235 240 | |
| Trp Gln Ile Ser Glu Ala Gly Thr Gly Val Ser Ser Ile Phe Ile Tyr | |
| 245 250 255 | |
| His Gly Glu Val Pro Gln Ala Ser Gln Val Pro Pro Pro Ser Gly Phe | |
| 260 265 270 | |
| Thr Val His Gly Leu Pro Thr Ser Pro Asp Arg Pro Gly Ser Thr Ser | |
| 275 280 285 | |
| Pro Phe Ala Pro Ser Ala Thr Asp Leu Pro Ser Met Pro Glu Pro Ala | |
| 290 295 300 | |
| Leu Thr Ser Arg Ala Asn Met Thr Glu His Lys Thr Ser Pro Thr Gln | |
| 305 310 315 320 | |
| Cys Pro Ala Ala Gly Glu Val Ser Asn Lys Leu Pro Lys Trp Pro Glu | |
| 325 330 335 | |
| Pro Val Glu Gln Phe Tyr Arg Ser Leu Gln Asp Thr Tyr Gly Ala Glu | |
| 340 345 350 | |
| Pro Ala Gly Pro Asp Gly Ile Leu Val Glu Val Asp Leu Val Gln Ala | |
| 355 360 365 | |
| Arg Leu Glu Arg Ser Ser Ser Lys Ser Leu Glu Arg Glu Leu Ala Thr | |
| 370 375 380 | |
| Pro Asp Trp Ala Glu Arg Gln Leu Ala Gln Gly Gly Leu Ala Glu Val | |
| 385 390 395 400 | |
| Leu Leu Ala Ala Lys Glu His Arg Arg Pro Arg Glu Thr Arg Val Ile | |
| 405 410 415 | |
| Ala Val Leu Gly Lys Ala Gly Gln Gly Lys Ser Tyr Trp Ala Gly Ala | |
| 420 425 430 | |
| Val Ser Arg Ala Trp Ala Cys Gly Arg Leu Pro Gln Tyr Asp Phe Val | |
| 435 440 445 | |
| Phe Ser Val Pro Cys His Cys Leu Asn Arg Pro Gly Asp Ala Tyr Gly | |
| 450 455 460 | |
| Leu Gln Asp Leu Leu Phe Ser Leu Gly Pro Gln Pro Leu Val Ala Ala | |
| 465 470 475 480 | |
| Asp Glu Val Phe Ser His Ile Leu Lys Arg Pro Asp Arg Val Leu Leu | |
| 485 490 495 | |
| Ile Leu Asp Gly Phe Glu Glu Leu Glu Ala Gln Asp Gly Phe Leu His | |
| 500 505 510 | |
| Ser Thr Cys Gly Pro Ala Pro Ala Glu Pro Cys Ser Leu Arg Gly Leu | |
| 515 520 525 | |
| Leu Ala Gly Leu Phe Gln Lys Lys Leu Leu Arg Gly Cys Thr Leu Leu | |
| 530 535 540 | |
| Leu Thr Ala Arg Pro Arg Gly Arg Leu Val Gln Ser Leu Ser Lys Ala | |
| 545 550 555 560 | |
| Asp Ala Leu Phe Glu Leu Ser Gly Phe Ser Met Glu Gln Ala Gln Ala | |
| 565 570 575 | |
| Tyr Val Met Arg Tyr Phe Glu Ser Ser Gly Met Thr Glu His Gln Asp | |
| 580 585 590 | |
| Arg Ala Leu Thr Leu Leu Arg Asp Arg Pro Leu Leu Leu Ser His Ser | |
| 595 600 605 | |
| His Ser Pro Thr Leu Cys Arg Ala Val Cys Gln Leu Ser Glu Ala Leu | |
| 610 615 620 | |
| Leu Glu Leu Gly Glu Asp Ala Lys Leu Pro Ser Thr Leu Thr Gly Leu | |
| 625 630 635 640 | |
| Tyr Val Gly Leu Leu Gly Arg Ala Ala Leu Asp Ser Pro Pro Gly Ala | |
| 645 650 655 | |
| Leu Ala Glu Leu Ala Lys Leu Ala Trp Glu Leu Gly Arg Arg His Gln | |
| 660 665 670 | |
| Ser Thr Leu Gln Glu Asp Gln Phe Pro Ser Ala Asp Val Arg Thr Trp | |
| 675 680 685 | |
| Ala Met Ala Lys Gly Leu Val Gln His Pro Pro Arg Ala Ala Glu Ser | |
| 690 695 700 | |
| Glu Leu Ala Phe Pro Ser Phe Leu Leu Gln Cys Phe Leu Gly Ala Leu | |
| 705 710 715 720 | |
| Trp Leu Ala Leu Ser Gly Glu Ile Lys Asp Lys Glu Leu Pro Gln Tyr | |
| 725 730 735 | |
| Leu Ala Leu Thr Pro Arg Lys Lys Arg Pro Tyr Asp Asn Trp Leu Glu | |
| 740 745 750 | |
| Gly Val Pro Arg Phe Leu Ala Gly Leu Ile Phe Gln Pro Pro Ala Arg | |
| 755 760 765 | |
| Cys Leu Gly Ala Leu Leu Gly Pro Ser Ala Ala Ala Ser Val Asp Arg | |
| 770 775 780 | |
| Lys Gln Lys Val Leu Ala Arg Tyr Leu Lys Arg Leu Gln Pro Gly Thr | |
| 785 790 795 800 | |
| Leu Arg Ala Arg Gln Leu Leu Glu Leu Leu His Cys Ala His Glu Ala | |
| 805 810 815 | |
| Glu Glu Ala Gly Ile Trp Gln His Val Val Gln Glu Leu Pro Gly Arg | |
| 820 825 830 | |
| Leu Ser Phe Leu Gly Thr Arg Leu Thr Pro Pro Asp Ala His Val Leu | |
| 835 840 845 | |
| Gly Lys Ala Leu Glu Ala Ala Gly Gln Asp Phe Ser Leu Asp Leu Arg | |
| 850 855 860 | |
| Ser Thr Gly Ile Cys Pro Ser Gly Leu Gly Ser Leu Val Gly Leu Ser | |
| 865 870 875 880 | |
| Cys Val Thr Arg Phe Arg Ala Ala Leu Ser Asp Thr Val Ala Leu Trp | |
| 885 890 895 | |
| Glu Ser Leu Gln Gln His Gly Glu Thr Lys Leu Leu Gln Ala Ala Glu | |
| 900 905 910 | |
| Glu Lys Phe Thr Ile Glu Pro Phe Lys Ala Lys Ser Leu Lys Asp Val | |
| 915 920 925 | |
| Glu Asp Leu Gly Lys Leu Val Gln Thr Gln Arg Thr Arg Ser Ser Ser | |
| 930 935 940 | |
| Glu Asp Thr Ala Gly Glu Leu Pro Ala Val Arg Asp Leu Lys Lys Leu | |
| 945 950 955 960 | |
| Glu Phe Ala Leu Gly Pro Val Ser Gly Pro Gln Ala Phe Pro Lys Leu | |
| 965 970 975 | |
| Val Arg Ile Leu Thr Ala Phe Ser Ser Leu Gln His Leu Asp Leu Asp | |
| 980 985 990 | |
| Ala Leu Ser Glu Asn Lys Ile Gly Asp Glu Gly Val Ser Gln Leu Ser | |
| 995 1000 1005 | |
| Ala Thr Phe Pro Gln Leu Lys Ser Leu Glu Thr Leu Asn Leu Ser | |
| 1010 1015 1020 | |
| Gln Asn Asn Ile Thr Asp Leu Gly Ala Tyr Lys Leu Ala Glu Ala | |
| 1025 1030 1035 | |
| Leu Pro Ser Leu Ala Ala Ser Leu Leu Arg Leu Ser Leu Tyr Asn | |
| 1040 1045 1050 | |
| Asn Cys Ile Cys Asp Val Gly Ala Glu Ser Leu Ala Arg Val Leu | |
| 1055 1060 1065 | |
| Pro Asp Met Val Ser Leu Arg Val Met Asp Val Gln Tyr Asn Lys | |
| 1070 1075 1080 | |
| Phe Thr Ala Ala Gly Ala Gln Gln Leu Ala Ala Ser Leu Arg Arg | |
| 1085 1090 1095 | |
| Cys Pro His Val Glu Thr Leu Ala Met Trp Thr Pro Thr Ile Pro | |
| 1100 1105 1110 | |
| Phe Ser Val Gln Glu His Leu Gln Gln Gln Asp Ser Arg Ile Ser | |
| 1115 1120 1125 | |
| Leu Arg | |
| 1130 | |

SEQ ID NO: 96
1/1 31/11
ATG AGC CTG TGG CTG CCC AGC GAG GCC ACC GTG TAC CTG CCC CCC GTG CCC GTG AGC AAG
61/21 91/31
GTG GTG AGC ACC GAC GAG TAC GTG GCC AGG ACC AAC ATC TAC TAC CAC GCC GGC ACC AGC
121/41 151/51
AGG CTG CTG GCC GTG GGC CAC CCC TAC TTC CCC ATC AAG AAG CCC AAC AAC AAC AAG ATC
181/61 211/71
CTG GTG CCC AAG GTG AGC GGC CTG CAG TAC AGG GTG TTC AGG ATC CAC CTG CCC GAC CCC
241/81 271/91
AAC AAG TTC GGC TTC CCC GAC ACC AGC TTC TAC AAC CCC GAC ACC CAG AGG CTG GTG TGG
301/101 331/111
GCC TGC GTG GGC GTG GAG GTG GGC AGG GGC CAG CCC CTG GGC GTG GGC ATC AGC GGC CAC
361/121 391/131
CCC CTG CTG AAC AAG CTG GAC GAC ACC GAG AAC GCC AGC GCC TAC GCC GCC AAC GCC GGC
421/141 451/151
GTG GAC AAC AGG GAG TGC ATC AGC ATG GAC TAC AAG CAG ACC CAG CTG TGC CTG ATC GGC
481/161 511/171
TGC AAG CCC CCC ATC GGC GAG CAC TGG GGC AAG GGC AGC CCC TGC ACC AAC GTG GCC GTG
541/181 571/191
AAC CCC GGC GAC TGC CCC CCC CTG GAG CTG ATC AAC ACC GTG ATC CAG GAC GGC GAC ATG
601/201 631/211
GTG GAC ACC GGC TTC GGC GCC ATG GAC TTC ACC ACC CTG CAG GCC AAC AAG AGC GAG GTG
661/221 691/231
CCC CTG GAC ATC TGC ACC AGC ATC TGC AAG TAC CCC GAC TAC ATC AAG ATG GTG AGC GAG
721/241 751/251
CCC TAC GGC GAC AGC CTG TTC TTC TAC CTG AGG AGG GAG CAG ATG TTC GTG AGG CAC CTG
781/261 811/271
TTC AAC AGG GCC GGC GCC GTG GGC GAG AAC GTG CCC GAC GAC CTG TAC ATC AAG GGC AGC
841/281 871/291
GGC AGC ACC GCC AAC CTG GCC AGC AGC AAC TAC TTC CCC ACC CCC AGC GGC AGC ATG GTG
901/301 931/311
ACC AGC GAC GCC CAG ATC TTC AAC AAG CCC TAC TGG CTG CAG AGG GCC CAG GGC CAC AAC
961/321 991/331
AAC GGC ATC TGC TGG GGC AAC CAG CTG TTC GTG ACC GTG GTG GAC ACC ACC AGG AGC ACC
1021/341 1051/351
AAC ATG AGC CTG TGC GCC GCC ATC AGC ACC AGC GAG ACC ACC TAC AAG AAC ACC AAC TTC
1081/361 1111/371
AAG GAG TAC CTG AGG CAC GGC GAG GAG TAC GAC CTG CAG TTC ATC TTC CAG CTG TGC AAG
1141/381 1171/391
ATC ACC CTG ACC GCC GAC GTG ATG ACC TAC ATC CAC AGC ATG AAC AGC ACC ATC CTG GAG
1201/401 1231/411
GAC TGG AAC TTC GGC CTG CAG CCC CCC CCC GGC GGC ACC CTG GAG GAC ACC TAC AGG TTC
1261/421 1291/431
GTG ACC AGC CAG GCC ATC GCC TGC CAG AAG CAC ACC CCC CCC GCC CCC AAG GAG GAC CCC
1321/441 1351/451
CTG AAG AAG TAC ACC TTC TGG GAG GTG AAC CTG AAG GAG AAG TTC AGC GCC GAC CTG GAC
1381/461 1411/471
CAG TTC CCC CTG GGC AGG AAG TTC CTG CTG CAG GCC GGC CTG AAG GCC AAG CCC AAG TTC
1441/481 1471/491
ACC CTG GGC AAG AGG AAG GCC ACC CCC ACC ACC AGC AGC ACC AGC ACC ACC GCC AAG AGG
1501/501
AAG AAG AGG AAG CTG TGA
SEQ ID NO: 97
1/1 31/11
Met ser leu trp leu pro ser glu ala thr val tyr leu pro pro val pro val ser lys
61/21 91/31
val val ser thr asp glu tyr val ala arg thr asn ile tyr tyr his ala gly thr ser
121/41 151/51
arg leu leu ala val gly his pro tyr phe pro ile lys lys pro asn asn asn lys ile
181/61 211/71
leu val pro lys val ser gly leu gln tyr arg val phe arg ile his leu pro asp pro
241/81 271/91
asn lys phe gly phe pro asp thr ser phe tyr asn pro asp thr gln arg leu val trp
301/101 331/111
ala cys val gly val glu val gly arg gly gln pro leu gly val gly ile ser gly his
361/121 391/131
pro leu leu asn lys leu asp asp thr glu asn ala ser ala tyr ala ala asn ala gly
421/141 451/151
val asp asn arg glu cys ile ser met asp tyr lys gln thr gln leu cys leu ile gly
481/161 511/171
cys lys pro pro ile gly glu his trp gly lys gly ser pro cys thr asn val ala val
541/181 571/191
asn pro gly asp cys pro pro leu glu leu ile asn thr val ile gln asp gly asp met
601/201 631/211
val asp thr gly phe gly ala met asp phe thr thr leu gln ala asn lys ser glu val
661/221 691/231
pro leu asp ile cys thr ser ile cys lys tyr pro asp tyr ile lys met val ser glu
721/241 751/251
pro tyr gly asp ser leu phe phe tyr leu arg arg glu gln met phe val arg his leu
781/261 811/271
phe asn arg ala gly ala val gly glu asn val pro asp asp leu tyr ile lys gly ser
841/281 871/291
gly ser thr ala asn leu ala ser ser asn tyr phe pro thr pro ser gly ser met val
901/301 931/311
thr ser asp ala gln ile phe asn lys pro tyr trp leu gln arg ala gln gly his asn
961/321 991/331
asn gly ile cys trp gly asn gln leu phe val thr val val asp thr thr arg ser thr
1021/341 1051/351
asn met ser leu cys ala ala ile ser thr ser glu thr thr tyr lys asn thr asn phe
1081/361 1111/371
lys glu tyr leu arg his gly glu glu tyr asp leu gln phe ile phe gln leu cys lys
1141/381 1171/391
ile thr leu thr ala asp val met thr tyr ile his ser met asn ser thr ile leu glu
1201/401 1231/411
asp trp asn phe gly leu gln pro pro pro gly gly thr leu glu asp thr tyr arg phe
1261/421 1291/431
val thr ser gln ala ile ala cys gln lys his thr pro pro ala pro lys glu asp pro
1321/441 1351/451
leu lys lys tyr thr phe trp glu val asn leu lys glu lys phe ser ala asp leu asp
1381/461 1411/471
gln phe pro leu gly arg lys phe leu leu gln ala gly leu lys ala lys pro lys phe
1441/481 1471/491
thr leu gly lys arg lys ala thr pro thr thr ser ser thr ser thr thr ala lys arg
1501/501
lys lys arg lys leu OPA
SEQ ID NO: 98

| | |
|---|---|
| 1 | atgtgcctgt atacacgggt cctgatatta cattaccatc tactacctct gtatggccca |
| 61 | ttgtatcacc cacggcccct gcctctacac agtatattgg tatacatggt acacattatt |
| 121 | atttgtggcc attatattat tttattccta agaaacgtaa acgtgttccc tatttttttg |
| 181 | cagatggctt tgtggcggcc tagtgacaat accgtatatc ttccacctcc ttctgtggca |
| 241 | agagttgtaa ataccgatga ttatgtgact cccacaagca tattttatca tgctggcagc |
| 301 | tctagattat taactgttgg taatccatat tttagggttc ctgcaggtgg tggcaataag |
| 361 | caggatattc ctaaggtttc tgcataccaa tatagagtat ttagggtgca gttacctgac |
| 421 | ccaaataaat ttggtttacc tgatactagt atttataatc ctgaaacaca acgtttagtg |
| 481 | tgggcctgtg ctggagtgga aattggccgt ggtcagcctt taggtgttgg ccttagtggg |
| 541 | catccatttt ataataaatt agatgacact gaaagttccc atgccgccac gtctaatgtt |
| 601 | tctgaggacg ttagggacaa tgtgtctgta gattataagc agacacagtt atgtattttg |
| 661 | ggctgtgccc ctgctattgg ggaacactgg gctaaaggca ctgcttgtaa atcgcgtcct |
| 721 | ttatcacagg gcgattgccc ccctttagaa cttaaaaaca cagttttgga agatggtgat |
| 781 | atggtagata ctggatatgg tgccatggac tttagtacat tgcaagatac taaatgtgag |
| 841 | gtaccattgg atatttgtca gtctatttgt aaatatcctg attatttaca aatgtctgca |
| 901 | gatccttatg gggattccat gtttttttgc ttacggcgtg agcagctttt tgctaggcat |
| 961 | ttttggaata gagcaggtac tatgggtgac actgtgcctc aatccttata tattaaaggc |
| 1021 | acaggtatgc ctgcttcacc tggcagctgt gtgtattctc cctctccaag tggctctatt |
| 1081 | gttacctctg actcccagtt gtttaataaa ccatattggt tacataaggc acagggtcat |
| 1141 | aacaatggtg tttgctggca taatcaatta tttgttactg tggtagatac cactcccagt |
| 1201 | accaatttaa caatatgtgc ttctacacag tctcctgtac ctgggcaata tgatgctacc |
| 1261 | aaatttaagc agtatagcag acatgttgag gaatatgatt tgcagtttat ttttcagttg |
| 1321 | tgtactatta ctttaactgc agatgttatg tcctatattc atagtatgaa tagcagtatt |
| 1381 | ttagaggatt ggaactttgg tgttcccccc cccccaacta ctagtttggt ggatacatat |
| 1441 | cgttttgtac aatctgttgc tattacctgt caaaaggatg ctgcaccggc tgaaaataag |
| 1501 | gatccctatg ataagttaaa gttttggaat gtggatttaa aggaaaagtt ttctttagac |
| 1561 | ttagatcaat atccccttgg acgtaaattt ttggttcagg ctggattgcg tcgcaagccc |
| 1621 | accataggcc ctcgcaaacg ttctgctcca tctgccacta cgtcttctaa acctgccaag |
| 1681 | cgtgtgcgtg tacgtgccag gaagtaa |

SEQ ID NO: 99

| | |
|---|---|
| 1 | mclytrvlil hyhllplygp lyhprplplh silvymvhii icghyiilfl rnvnvfpifl |
| 61 | qmalwrpsdn tvylpppsva rvvntddyvt ptsifyhags srlltvgnpy frvpagggnk |
| 121 | qdipkvsayq yrvfrvqlpd pnkfglpdts iynpetqrlv wacagveigr gqplgvglsg |
| 181 | hpfynklddt esshaatsnv sedvrdnvsv dykqtqlcil gcapaigehw akgtacksrp |
| 241 | lsqgdcpple lkntvledgd mvdtgygamd fstlqdtkce vpldicqsic kypdylqmsa |
| 301 | dpygdsmffc lrreqlfarh fwnragtmgd tvpqslyikg tgmpaspgsc vyspspsgsi |
| 361 | vtsdsqlfnk pywlhkaqgh nngvcwhnql fvtvvdttps tnlticastq spvpgqydat |
| 421 | kfkqysrhve eydlqfifql ctitltadvm syihsmnssi ledwnfgvpp ppttslvdty |
| 481 | rfvqsvaitc qkdaapaenk dpydklkfwn vdlkekfsld ldqyplgrkf lvqaglrrkp |
| 541 | tigprkrsap sattsskpak rvrvrark |

SEQ ID NO: 100

| | |
|---|---|
| 1 | atgtcttgtg gcctaaacga cgtaaacgtg tccactattt ctttgcagat ggctttgtgg |
| 61 | cggcctaatg aaagcaaggt atacctacct ccaacacctg tttcaaaggt gatcagtacg |
| 121 | gatgtctatg tcacgcggac taatgtgtat taccatggtg gcagttctag gcttctcact |
| 181 | gtgggtcatc catattactc tataaagaag agtaataata aggtggctgt gcccaaggta |
| 241 | tctgggtacc aatatcgtgt atttcacgtg aagttgccag atccaaataa gtttggcctg |
| 301 | cccgatgctg atttgtatga tccagatacc cagagacttc tgtgggcgtg cgtgggagta |
| 361 | gaggtgggcc gtgggcagcc tttgggtgtg ggtgtgtctg gtcacccata ttacaataga |
| 421 | ctggatgaca ctgaaaatgc acacacacct gatacagctg atgatggcag ggaaaacatt |
| 481 | tctatggatt ataaacagac acagctgttc attctgggct gcaaaccccc tattggtgag |
| 541 | cactggtcta agggtaccac ctgtaatggg tcttctgctg ctggtgactg cccgcccctc |
| 601 | caatttacta acacaactat tgaggacggg gatatggttg aaacagggtt cggtgccttg |
| 661 | gattttgcca ctctgcagtc aaataagtca gatgttcctt tggatatttg taccaatacc |
| 721 | tgtaaatatc ctgattatct gaagatggct gcagagcctt atggtgattc tatgttcttc |
| 781 | tcgctgcgta gggaacaaat gttcactcgt cattttttca atctgggtgg taagatgggt |
| 841 | gacaccatcc cggatgagtt atacattaaa agtacctcag ttccaactcc aggcagtcat |
| 901 | gtttatactt ccactcctag tggctctatg gtgtcctctg aacaacagtt gtttaataag |
| 961 | ccttactggc tacggagggc ccaagggcac aacaatggta tgtgctgggg caatagggtc |
| 1021 | tttctgactg tggtggacac cacacgtagc actaatgtat ctctgtgtgc cactgaggcg |
| 1081 | tctgatacta attataaggc taccaatttt aaggaatatc tcaggcatat ggaggaatat |
| 1141 | gatttgcagt tcatcttcca actgtgcaag ataaccctta ctcctgaaat tatggcctat |
| 1201 | atacataata tggatcccca gttgttagag gattggaact tcggtgtacc ccctccgccg |
| 1261 | tctgccagtt tacaggatac ctatagatat ttgcagtccc aggctattac atgtcaaaaa |
| 1321 | cctacacctc ctaagacccc taccgatccc tatgcctccc tgaccttttg ggatgtggat |
| 1381 | ctcagtgaaa gtttttccat ggatctggac caatttccct tgggtcgcaa gtttttgctg |
| 1441 | cagcgggggg ctatgcctac cgtgtctcgc aagcgcgccg ctgtttcggg gaccacgccg |
| 1501 | cccactagta aacgaaaacg ggtaaggcgt tag |

SEQ ID NO: 101

| | |
|---|---|
| 1 | mscglndvnv stislqmalw rpneskvylp ptpvskvist dvyvtrtnvy yhggssrllt |
| 61 | vghpyysikk snnkvavpkv sgyqyrvfhv klpdpnkfgl pdadlydpdt qrllwacvgv |
| 121 | evgrgqplgv gvsghpyynr lddtenahtp dtaddgreni smdykqtqlf ilgckppige |
| 181 | hwskgttcng ssaagdcppl qftnttiedg dmvetgfgal dfatlqsnks dvpldictnt |
| 241 | ckypdylkma aepygdsmff slrreqmftr hffnlggkmg dtipdelyik stsvptpgsh |
| 301 | vytstpsgsm vsseqqlfnk pywlrraqgh nngmcwgnrv fltvvdttrs tnvslcatea |
| 361 | sdtnykatnf keylrhmeey dlqfifqlck itltpeimay ihnmdpqlle dwnfgvpppp |
| 421 | saslqdtyry lqsqaitcqk ptppktptdp yasltfwdvd lsesfsmdld qfplgrkfll |
| 481 | qrgamptvsr kraavsgttp ptskrkrvrr |

SEQ ID NO: 102
1/1 31/11
ATG AGG CAC AAG AGG AGC GCC AAG AGG ACC AAG AGG GCC AGC GCC ACC CAG CTG TAC AAG
61/21 91/31
ACC TGC AAG CAG GCC GGC ACC TGC CCC CCC GAC ATC ATC CCC AAG GTG GAG GGC AAG ACC
21/41 151/51
ATC GCC GAC CAG ATC CTG CAG TAC GGC AGC ATG GGC GTG TTC TTC GGC GGC CTG GGC ATC
181/61 211/71
GGC ACC GGC AGC GGC ACC GGC GGC AGG ACC GGC TAC ATC CCC CTG GGC ACC AGG CCC CCC
241/81 271/91
ACC GCC ACC GAC ACC CTG GCC CCC GTG AGG CCC CCC CTG ACC GTG GAC CCC GTG GGC CCC
301/101 331/111
AGC GAC CCC AGC ATC GTG AGC CTG GTG GAG GAG ACC AGC TTC ATC GAC GCC GGC GCC CCC
361/121 391/131
ACC AGC GTG CCC AGC ATC CCC CCC GAC GTG AGC GGC TTC AGC ATC ACC ACC AGC ACC GAC
21/141 451/151
ACC ACC CCC GCC ATC CTG GAC ATC AAC AAC ACC GTG ACC ACC GTG ACC ACC CAC AAC AAC
81/161 511/171
CCC ACC TTC ACC GAC CCC AGC GTG CTG CAG CCC CCC ACC CCC GCC GAG ACC GGC GGC CAC
541/181 571/191
TTC ACC CTG AGC AGC AGC ACC ATC AGC ACC CAC AAC TAC GAG GAG ATC CCC ATG GAC ACC
601/201 631/211
TTC ATC GTG AGC ACC AAC CCC AAC ACC GTG ACC AGC AGC ACC CCC ATC CCC GGC AGC AGG
661/221 691/231
CCC GTG GCC AGG CTG GGC CTG TAC AGC AGG ACC ACC CAG CAG GTG AAG GTG GTG GAC CCC
721/241 751/251
GCC TTC GTG ACC ACC CCC ACC AAG CTG ATC ACC TAC GAC AAC CCC GCC TAC GAG GGC ATC
781/261 811/271
GAC GTG GAC AAC ACC CTG TAC TTC AGC AGC AAC GAC AAC AGC ATC AAC ATC GCC CCC GAC
841/281 871/291
CCC GAC TTC CTG GAC ATC GTG GCC CTG CAC AGG CCC GCC CTG ACC AGC AGG AGG ACC GGC
901/301 931/311
ATC AGG TAC AGC AGG ATC GGC AAC AAG CAG ACC CTG AGG ACC AGG AGC GGC AAG AGC ATC
961/321 991/331
GGC GCC AAG GTG CAC TAC TAC TAC GAC CTG AGC ACC ATC GAC CCC GCC GAG GAG ATC GAG
1021/341 1051/351
CTG CAG ACC ATC ACC CCC AGC ACC TAC ACC ACC ACC AGC CAC GCC GCC AGC CCC ACC AGC
081/361 1111/371
ATC AAC AAC GGC CTG TAC GAC ATC TAC GCC GAC GAC TTC ATC ACC GAC ACC AGC ACC ACC
1141/381 1171/391
CCC GTG CCC AGC GTG CCC AGC ACC AGC CTG AGC GGC TAC ATC CCC GCC AAC ACC ACC ATC
1201/401 1231/411
CCC TTC GGT GGC GCC TAC AAC ATC CCC CTG GTG AGC GGC CCC GAC ATC CCC ATC AAC ATC
1261/421 1291/431
ACC GAC CAG GCC CCC AGC CTG ATC CCC ATC GTG CCC GGC AGC CCC CAG TAC ACC ATC ATC
1321/441 1351/451
GCC GAC GCC GGC GAC TTC TAC CTG CAC CCC AGC TAC TAC ATG CTG AGG AAG AGG AGG AAG
1381/461 1411/471
AGG CTG CCC TAC TTC TTC AGC GAC GTG AGC CTG GCC GCC TGA
SEQ ID NO: 103
1/1 31/11
Met arg his lys arg ser ala lys arg thr lys arg ala ser ala thr gln leu tyr lys
61/21 91/31
thr cys lys gln ala gly thr cys pro pro asp ile ile pro lys val glu gly lys thr
121/41 151/51
ile ala asp gln ile leu gln tyr gly ser met gly val phe phe gly gly leu gly ile
181/61 211/71
gly thr gly ser gly thr gly gly arg thr gly tyr ile pro leu gly thr arg pro pro
241/81 271/91
thr ala thr asp thr leu ala pro val arg pro pro leu thr val asp pro val gly pro
301/101 331/111
ser asp pro ser ile val ser leu val glu glu thr ser phe ile asp ala gly ala pro
361/121 391/131
thr ser val pro ser ile pro pro asp val ser gly phe ser ile thr thr ser thr asp
421/141 451/151
thr thr pro ala ile leu asp ile asn asn thr val thr thr val thr thr his asn asn
481/161 511/171
pro thr phe thr asp pro ser val leu gln pro pro thr pro ala glu thr gly gly his
541/181 571/191
phe thr leu ser ser ser thr ile ser thr his asn tyr glu glu ile pro met asp thr
601/201 631/211
phe ile val ser thr asn pro asn thr val thr ser ser thr pro ile pro gly ser arg
661/221 691/231
pro val ala arg leu gly leu tyr ser arg thr thr gln gln val lys val val asp pro
721/241 751/251
ala phe val thr thr pro thr lys leu ile thr tyr asp asn pro ala tyr glu gly ile
781/261 811/271
asp val asp asn thr leu tyr phe ser ser asn asp asn ser ile asn ile ala pro asp
841/281 871/291
pro asp phe leu asp ile val ala leu his arg pro ala leu thr ser arg arg thr gly
901/301 931/311
ile arg tyr ser arg ile gly asn lys gln thr leu arg thr arg ser gly lys ser ile
961/321 991/331
gly ala lys val his tyr tyr tyr asp leu ser thr ile asp pro ala glu glu ile glu
1021/341 1051/351
leu gln thr ile thr pro ser thr tyr thr thr thr ser his ala ala ser pro thr ser
1081/361 1111/371
ile asn asn gly leu tyr asp ile tyr ala asp asp phe ile thr asp thr ser thr thr
1141/381 1171/391
pro val pro ser val pro ser thr ser leu ser gly tyr ile pro ala asn thr thr ile
1201/401 1231/411
pro phe gly gly ala tyr asn ile pro leu val ser gly pro asp ile pro ile asn ile
1261/421 1291/431
thr asp gln ala pro ser leu ile pro ile val pro gly ser pro gln tyr thr ile ile
1321/441 1351/451
ala asp ala gly asp phe tyr leu his pro ser tyr tyr met leu arg lys arg arg lys
1381/461 1411/471
arg leu pro tyr phe phe ser asp val ser leu ala ala OPA
SEQ ID NO: 104

| | |
|---|---|
| 1 | atggtatccc accgtgccgc acgacgcaaa cgggcttcgg taactgactt atataaaaca |
| 61 | tgtaaacaat ctggtacatg tccacctgat gttgttccta aggtggaggg caccacgtta |
| 121 | gcagataaaa tattgcaatg gtcaagcctt ggtatatttt tgggtggact tggcataggt |
| 181 | actggcagtg gtacaggggg tcgtacaggg tacattccat tgggtgggcg ttccaataca |
| 241 | gtggtggatg ttggtcctac acgtccccca gtggttattg aacctgtggg ccccacagac |
| 301 | ccatctattg ttacattaat agaggactcc agtgtggtta catcaggtgc acctaggcct |
| 361 | acgtttactg gcacgtctgg gtttgatata acatctgcgg gtacaactac acctgcggtt |
| 421 | ttggatatca caccttcgtc tacctctgtg tctatttcca caaccaattt taccaatcct |
| 481 | gcattttctg atccgtccat tattgaagtt ccacaaactg gggaggtggc aggtaatgta |
| 541 | tttgttggta cccctacatc tggaacacat gggtatgagg aaataccttt acaaacattt |
| 601 | gcttcttctg gtacggggga ggaacccatt agtagtaccc cattgcctac tgtgcggcgt |
| 661 | gtagcaggtc cccgccttta cagtagggcc taccaacaag tgtcagtggc taaccctgag |
| 721 | tttcttacac gtccatcctc tttaattaca tatgacaacc cggcctttga gcctgtggac |
| 781 | actacattaa catttgatcc tcgtagtgat gttcctgatt cagattttat ggatattatc |
| 841 | cgtctacata ggcctgcttt aacatccagg cgtgggactg ttcgctttag tagattaggt |
| 901 | caacgggcaa ctatgtttac ccgcagcggt acacaaatag gtgctagggt tcacttttat |
| 961 | catgatataa gtcctattgc accttcccca gaatatattg aactgcagcc tttagtatct |
| 1021 | gccacggagg acaatgactt gtttgatata tatgcagatg acatggaccc tgcagtgcct |
| 1081 | gtaccatcgc gttctactac ctcctttgca ttttttaaat attcgcccac tatatcttct |
| 1141 | gcctcttcct atagtaatgt aacggtccct ttaacctcct cttgggatgt gcctgtatac |
| 1201 | acgggtcctg atattacatt accatctact acctctgtat ggcccattgt atcacccacg |
| 1261 | gcccctgcct ctacacagta tattggtata catggtacac attattattt gtggccatta |
| 1321 | tattatttta ttcctaagaa acgtaaacgt gttccctatt tttttgcaga tggctttgtg |
| 1381 | gcggcctag |

SEQ ID NO: 105

| | |
|---|---|
| 1 | mvshraarrk rasvtdlykt ckqsgtcppd vvpkvegttl adkilqwssl giflgglgig |
| 61 | tgsgtggrtg yiplggrsnt vvdvgptrpp vviepvgptd psivtlieds svvtsgaprp |
| 121 | tftgtsgfdi tsagtttpav lditpsstsv sisttnftnp afsdpsiiev pqtgevagnv |
| 181 | fvgtptsgth gyeeiplqtf assgtgeepi sstplptvrr vagprlysra yqqvsvanpe |
| 241 | fltrpsslit ydnpafepvd ttltfdprsd vpdsdfmdii rlhrpaltsr rgtvrfsrlg |
| 301 | qratmftrsg tqigarvhfy hdispiapsp eyielqplvs atedndlfdi yaddmdpavp |
| 361 | vpsrsttsfa ffkysptiss assysnvtvp ltsswdvpvy tgpditlpst tsvwpivspt |
| 421 | apastqyigi hgthyylwpl yyfipkkrkr vpyffadgfv aa |

SEQ ID NO: 106

| | |
|---|---|
| 1 | atgtctgttg gtgattctta tcctaatcgc ctttttattg ttgatgtttt atgtccgttt |
| 61 | gttaaaccac acctaacacc cccacttttt tatattgttt tgatacattt tcattttgat |
| 121 | acatttgtgt tttttttgta tttgctgcgt tttaataaac gtgcaaccat gtctatacgt |
| 181 | gccaagcgtc gaaagcgcgc ctcccccaca gacctctatc gtacctgcaa gcaggcaggt |
| 241 | acctgccccc cagacattat cccaagagtg gaacagaaca ctttagcaga taaaatcctt |
| 301 | aagtggggca gtttaggtgt gttttttggg ggtctaggta taggcaccgg cagcggcaca |
| 361 | ggggggcgta ctgggtacat tcctgtaggt tcgcgaccca ccactgtagt tgacattggt |
| 421 | ccaacgccca ggccgcctgt tatcattgaa cctgtggggg cctctgaacc ctctattgtc |
| 481 | actttggtgg aggactctag catcattaac gcaggagcgt cacatcccac ctttactggt |
| 541 | actggtggct tcgaagtgac aacctccacc gttacagacc ccgccgtctt ggatatcacc |
| 601 | ccctcaggta ccagtgtgca ggtcagcagc agtagctttc ttaacccact atacactgag |
| 661 | ccagctattg tggaggctcc ccaaacaggg gaagtatctg gccatgtact tgttagtaca |
| 721 | gccacctcag ggtctcatgg ctatgaggaa ataccaatgc agacgtttgc cacgtcgggg |
| 781 | ggcagcggta cagagcctat cagtagcaca cccctccctg gcgtgcggag agttgccgga |
| 841 | ccccgcctgt acagtagagc caatcagcaa gtgcaagtca gggatcctgc gtttcttgca |
| 901 | aggcctgctg atctagtaac atttgacaat cctgtgtatg acccagagga aactataata |
| 961 | tttcagcatc cagacttgca tgagccaccg gatcctgatt ttttggacat agtggcgttg |
| 1021 | catcgtcccg ccctcacgtc cagaaggggt actgtccgtt ttagtaggtt gggacgcagg |
| 1081 | gctacactcc gcacccgtag tggtaaacaa attggggcac gggtgcactt ctatcatgat |
| 1141 | attagcccta taggtactga ggagttggag atggagccac tgttgccccc agcttctact |
| 1201 | gataacacag atatgttata tgatgtttat gctgattcgg atgtccttca gccattgctt |
| 1261 | gatgagttac ccgccgcccc tcgcggttca ctctctctgg ctgacactgc tgtgtctgcc |
| 1321 | acctccgcat ctacactacg ggggtccact actgtccctt tatcaagtgg tattgatgtg |
| 1381 | cctgtgtaca ccggtcctga cattgaacca cccaatgttc ctggcatggg acctctgatt |
| 1441 | cctgtggctc catccttacc atcgtctgtg tacatatttg ggggagatta ttatttgatg |
| 1501 | ccaagttatg tcttgtggcc taaacgacgt aaacgtgtcc actatttctt tgcagatggc |
| 1561 | tttgtggcgg cctaa |

SEQ ID NO: 107

| | |
|---|---|
| 1 | msvgdsypnr lfivdvlcpf vkphltpplf yivlihfhfd tfvfflyllr fnkratmsir |
| 61 | akrrkraspt dlyrtckqag tcppdiiprv eqntladkil kwgslgvffg glgigtgsgt |
| 121 | ggrtgyipvg srpttvvdig ptprppviie pvgasepsiv tlvedssiin agashptftg |
| 181 | tggfevttst vtdpavldit psgtsvqvss ssflnplyte paiveapqtg evsghvlvst |
| 241 | atsgshgyee ipmqtfatsg gsgtepisst plpgvrrvag prlysranqq vqvrdpafla |
| 301 | rpadlvtfdn pvydpeetii fqhpdlhepp dpdfldival hrpaltsrrg tvrfsrlgrr |
| 361 | atlrtrsgkq igarvhfyhd ispigteele mepllppast dntdmlydvy adsdvlqpll |
| 421 | delpaaprgs lsladtavsa tsastlrgst tvplssgidv pvytgpdiep pnvpgmgpli |
| 481 | pvapslpssv yifggdyylm psyvlwpkrr krvhyffadg fvaa |

SEQ ID NO: 108

| | |
|---|---|
| 1 | atggagctga ggccctggtt gctatgggtg gtagcagcaa caggaacctt ggtcctgcta |
| 61 | gcagctgatg ctcagggcca gaaggtcttc accaacacgt gggctgtgcg catccctgga |
| 121 | ggcccagcgg tggccaacag tgtggcacgg aagcatgggt tcctcaacct gggccagatc |
| 181 | ttcggggact attaccactt ctggcatcga ggagtgacga agcggtccct gtcgcctcac |
| 241 | cgcccgcggc acagccggct gcagagggag cctcaagtac agtggctgga acagcaggtg |
| 301 | gcaaagcgac ggactaaacg ggacgtgtac caggagccca cagaccccaa gtttcctcag |
| 361 | cagtggtacc tgtctggtgt cactcagcgg gacctgaatg tgaaggcggc ctgggcgcag |
| 421 | ggctacacag ggcacggcat tgtggtctcc attctggacg atggcatcga gaagaaccac |
| 481 | ccggacttgg caggcaatta tgatcctggg gccagttttg atgtcaatga ccaggaccct |
| 541 | gacccccagc ctcggtacac acagatgaat gacaacaggc acggcacacg gtgtgcgggg |
| 601 | gaagtggctg cggtggccaa caacggtgtc tgtggtgtag gtgtggccta caacgcccgc |
| 661 | attggagggg tgcgcatgct ggatggcgag gtgacagatg cagtggaggc acgctcgctg |
| 721 | ggcctgaacc ccaaccacat ccacatctac agtgccagct ggggccccga ggatgacggc |
| 781 | aagacagtgg atgggccagc ccgcctcgcc gaggaggcct tcttccgtgg ggttagccag |
| 841 | ggccgagggg ggctgggctc catctttgtc tgggcctcgg ggaacggggg ccgggaacat |
| 901 | gacagctgca actgcgacgg ctacaccaac agtatctaca cgctgtccat cagcagcgcc |
| 961 | acgcagtttg gcaacgtgcc gtggtacagc gaggcctgct cgtccacact ggccacgacc |
| 1021 | tacagcagtg gcaaccagaa tgagaagcag atcgtgacga ctgacttgcg gcagaagtgc |
| 1081 | acggagtctc acacgggcac ctcagcctct gcccccttag cagccggcat cattgctctc |
| 1141 | accctggagg ccaataagaa cctcacatgg cgggacatgc aacacctggt ggtacagacc |
| 1201 | tcgaagccag cccacctcaa tgccaacgac tgggccacca atggtgtggg ccggaaagtg |
| 1261 | agccactcat atggctacgg gcttttggac gcaggcgcca tggtggccct ggcccagaat |
| 1321 | tggaccacag tggcccccca gcggaagtgc atcatcgaca tcctcaccga gcccaaagac |
| 1381 | atcgggaaac ggctcgaggt gcggaagacc gtgaccgcgt gcctgggcga gcccaaccac |
| 1441 | atcactcggc tggagcacgc tcaggcgcgg ctcaccctgt cctataatcg ccgtggcgac |
| 1501 | ctggccatcc acctggtcag ccccatgggc acccgctcca ccctgctggc agccaggcca |
| 1561 | catgactact ccgcagatgg gtttaatgac tgggccttca tgacaactca ttcctgggat |
| 1621 | gaggatccct ctggcgagtg ggtcctagag attgaaaaca ccagcgaagc caacaactat |
| 1681 | gggacgctga ccaagttcac cctcgtactc tatggcaccg cccctgaggg gctgcccgta |
| 1741 | cctccagaaa gcagtggctg caagaccctc acgtccagtc aggcctgtgt ggtgtgcgag |
| 1801 | gaaggcttct ccctgcacca gaagagctgt gtccagcact gccctccagg gttcgccccc |
| 1861 | caagtcctcg atacgcacta tagcaccgag aatgacgtgg agaccatccg ggccagcgtc |
| 1921 | tgcgccccct gccacgcctc atgtgccaca tgccaggggc cggccctgac agactgcctc |
| 1981 | agctgcccca gccacgcctc cttggaccct gtggagcaga cttgctcccg gcaaagccag |
| 2041 | agcagccgag agtccccgcc acagcagcag ccacctcggc tgcccccgga ggtggaggcg |
| 2101 | gggcaacggc tgcgggcagg gctgctgccc tcacacctgc ctgaggtggt ggccggcctc |
| 2161 | agctgcgcct tcatcgtgct ggtcttcgtc actgtcttcc tggtcctgca gctgcgctct |
| 2221 | ggctttagtt ttcggggggt gaaggtgtac accatggacc gtggcctcat ctcctacaag |
| 2281 | gggctgcccc ctgaagcctg gcaggaggag tgcccgtctg actcagaaga ggacgagggc |
| 2341 | cggggcgaga ggaccgcctt tatcaaagac cagagcgccc tctga |

SEQ ID NO: 109

| | |
|---|---|
| 1 | melrpwllwv vaatgtlvll aadaqgqkvf tntwavripg gpavansvar khgflnlgqi |
| 61 | fgdyyhfwhr gvtkrslsph rprhsrlqre pqvqwleqqv akrrtkrdvy qeptdpkfpq |
| 121 | qwylsgvtqr dlnvkaawaq gytghgivvs ilddgieknh pdlagnydpg asfdvndqdp |
| 181 | dpqprytqmn dnrhgtrcag evaavanngv cgvgvaynar iggvrmldge vtdavearsl |
| 241 | glnpnhihiy saswgpeddg ktvdgparla eeaffrgvsq grgglgsifv wasgnggreh |
| 301 | dscncdgytn siytlsissa tqfgnvpwys eacsstlatt yssgnqnekq ivttdlrqkc |
| 361 | teshtgtsas aplaagiial tleanknltw rdmqhlvvqt skpahlnand watngvgrkv |
| 421 | shsygyglld agamvalaqn wttvapqrkc iidiltepkd igkrlevrkt vtaclgepnh |
| 481 | itrlehaqar ltlsynrrgd laihlvspmg trstllaarp hdysadgfnd wafmtthswd |
| 541 | edpsgewvle ientseanny gtltkftlvl ygtapeglpv ppessgcktl tssqacvvce |
| 601 | egfslhqksc vqhcppgfap qvldthyste ndvetirasv capchascat cqgpaltdcl |
| 661 | scpshasldp veqtcsrqsq ssresppqqq pprlppevea gqrlragllp shlpevvagl |
| 721 | scafivlvfv tvflvlqlrs gfsfrgvkvy tmdrglisyk glppeawqee cpsdseedeg |
| 781 | rgertafikd qsal |

SEQ ID NO: 110
AATGGACCAGTTCTAATGT
SEQ ID NO: 111
GTCAGCCCTAAATTCTTC
SEQ ID NO: 112
TAATACGACTCACTATAGGG
SEQ ID NO: 113
TAGAAGGCACAGTCGAGG
SEQ ID NO: 114
ATGGTGAGCAAGGGCGAGGAG
SEQ ID NO: 115
CTTGTACAGCTCGTCCATGCC
SEQ ID NO: 116
CCGGATCCTGGGAAGCTTGTCATCAACGG
SEQ ID NO: 117
GGCTCGAGGCAGTGATGGCATGGACTG

1. A method of enhancing an antigen-specific immune response in a mammal, comprising administering to the subject an effective amount of a papillomavirus pseudovirion, wherein the papillomavirus pseudovirion comprises at least one papillomavirus capsid protein encapsidating a naked DNA vaccine, wherein the naked DNA vaccine comprises a first nucleic acid encoding at least one antigen, thereby enhancing the antigen specific immune response relative to administration of the naked DNA vaccine.

2. The method of claim 1, wherein the papillomavirus pseudovirion comprises at least one furin-cleaved papillomavirus capsid protein.

3. The method of claim 1, wherein the at least one papillomavirus capsid protein is a papillomavirus L1 protein and a papillomavirus L2 protein.

4. The method of claim 3, wherein the papillomavirus L1 and L2 proteins are derived from HPV-2, HPV-16, or HPV-18.

5. The method of claim 4, wherein the papillomavirus L1 protein comprises an amino acid sequence selected from the group consisting of SEQ ID NOs:97, 99, and 101, and the papillomavirus L2 protein comprises an amino acid sequence selected from the group consisting of SEQ ID NOs: 103, 105 and 107.

6. The method of claim 1, wherein the antigen is a tumor-associated antigen (TAA).

7. The method of claim 1, wherein the antigen is foreign to the mammal.

8. The method of claim 1, wherein the antigen is selected from the group consisting of ovalbumin, HPV E6, and HPV E7.

9. The method of claim 8, wherein the antigen comprises an ovalbumin protein comprising an amino acid sequence at least 90% identical to the amino acid sequence of SEQ ID NO:9.

10. The method of claim 8, wherein the antigen comprises an HPV E6 protein comprising an amino acid sequence at least 90% identical to the amino acid sequence of SEQ ID NO: 5 or a non-oncogenic mutant thereof.

11. The method of claim 8, wherein the antigen comprises an HPV E7 protein comprising an amino acid sequence at least 90% identical to the amino acid sequence of SEQ ID NO:2 or a non-oncogenic mutant thereof.

12. The method of claim 1, wherein the DNA vaccine further comprises a second nucleic acid encoding a fusion protein comprising an Ii protein, wherein the class II-associated Ii peptide (CLIP) region is replaced with the Pan HLA-DR reactive epitope (PADRE).

13. The method of claim 1, wherein the DNA vaccine further comprises a second nucleic acid encoding a fusion protein comprising an Ii protein, wherein the class II-associated Ii peptide (CLIP) region is replaced with the Pan HLA-DR reactive epitope (PADRE).

14. The method of claim 1, wherein the DNA vaccine further comprises a second nucleic acid that is (i) a siNA or (ii) DNA that encodes said siNA, wherein said siNA has a sequence that is sufficiently complementary to target the sequence of mRNA that encodes a pro-apoptotic protein expressed in a dendritic cell (DC) and results in inhibition of or loss of expression of said mRNA, thereby inhibiting apoptosis and increasing survival of DCs.

15. The method of claim 14, wherein said pro-apoptotic protein is selected from the group consisting of one or more of (a) Bak, (b) Bax, (c) caspase-8, (d) caspase-9 and (e) caspase-3.

16. The method of claim 1, wherein the DNA vaccine further comprises a second nucleic acid encoding an anti-apoptotic polypeptide.

17. The method of claim 16, wherein said anti-apoptotic polypeptide is selected from the group consisting of (a) BCL-x1, (b) BCL2, (c) XIAP. (d) FLICEc-s, (e) dominant-negative caspase-8, (f) dominant negative caspase-9, (g) SPI-6, and (h) a functional homologue or derivative of any of (a)-(g).

18. The method of claim 1, wherein the DNA vaccine further comprises a second nucleic acid encoding an immunogenicity potentiating peptide (IPP), wherein the IPP acts in potentiating an immune response by promoting:
(a) processing of the linked antigenic polypeptide via the MHC class I pathway or targeting of a cellular compartment that increases said processing;
(b) development, accumulation or activity of antigen presenting cells or targeting of antigen to compartments of said antigen presenting cells leading to enhanced antigen presentation;
(c) intercellular transport and spreading of the antigen; or
(d) any combination of (a)-(c).

19. The method of claim 18, wherein the IPP is:
(a) the sorting signal of the lysosome-associated membrane protein type 1 (Sig/LAMP-1);
(b) a mycobacterial HSP70 polypeptide, the C-terminal domain thereof, or a functional homologue or derivative of said polypeptide or domain;
(c) a viral intercellular spreading protein selected from the group of herpes simplex virus-1 VP22 protein, Marek's disease virus UL49 protein or a functional homologue or derivative thereof;
(d) an endoplasmic reticulum chaperone polypeptide selected from the group of calreticulin or a domain thereof, ER60, GRP94, gp96, or a functional homologue or derivative thereof;
(e) domain II of Pseudomonas exotoxin ETA or a functional homologue or derivative thereof;
(f) a polypeptide that targets the centrosome compartment of a cell selected from γ-tubulin or a functional homologue or derivative thereof; or
(g) a polypeptide that stimulates DC precursors or activates DC activity selected from the group consisting of GM-CSF, Flt3-ligand extracellular domain, or a functional homologue or derivative thereof.

20. The method of claim 12, wherein the first and second nucleic acid sequences are comprised within at least one expression vector and are operatively linked to (a) a promoter; and (b) optionally, additional regulatory sequences that regulate expression of said nucleic acids in a eukaryotic cell.

21. The method of claim 20, wherein the first and second nucleic acid are operably linked either directly or via a linker.

22. The method of claim 1, wherein the nucleic acid composition is papillomavirus pseudovirion is administered intradermally, intraperitoneally, or intravenously.

23. The method of claim 1, wherein the papillomavirus pseudovirion is administered to the subject by:
(a) priming the mammal by administering to the mammal an effective amount of the papillomavirus pseudovirion; and
(b) boosting the mammal by administering to the mammal an effective amount of the papillomavirus pseudovirion,
thereby inducing or enhancing the antigen-specific immune response.

24. The method of claim 23, wherein the papillomavirus pseudo virions administered in steps (a) and (b) comprise the same type of capsid protein composition to thereby produce homologous vaccination.

25. The method of claim 23, wherein the papillomavirus pseudo virions administered in steps (a) and (b) comprise different types of capsid protein compositions to thereby produce heterologous vaccination.

26. The method of claim 23, wherein step (a) and/or step (b) is repeated at least once.

27. The method of claim 1, wherein the antigen-specific immune response is mediated at least in part by CD8⁺ cytotoxic T lymphocytes (CTL).

28. The method of claim 1, wherein the pseudovirions infect bone marrow-derived dendritic cells (BMDCs).

29. The method of claim 28, wherein the BMDCs are selected from the group consisting of B220+ cells and CD11 c+ cells.

30. The method of claim 1, further comprising administering an effective amount of a chemotherapeutic agent.

31. The method of claim 1, further comprising screening the mammal for the presence of antibodies against the antigen.

32. The method of claim 1, wherein the mammal is a human.

33. The method of claim 1, wherein the mammal is afflicted with cancer.
